SEQUENCE LISTING (266 sequences)

SEQ ID NO: 1 - protein, 185 aa, Homo sapiens
GDSSPDTFSDGLSSSTLPDDHSSYTVPGYMQDLEVEQALTPALSPCAVSSTLPDWHIPVEVVPDSTSDLYNFQVSPMPSTSEATTDEDEEGKLPEDIMKLLEQSEWQPTNVDGKGYLLNEPGVQPTSVYGDFSCKEEPEIDSPGGDIGLSLQRVFTDLKNMDATWLDSLLTPVRLPSIQAIPCAP

SEQ ID NO: 2 - DNA, 558 bp, Homo sapiens
ggggattccagccctgataccttctctgatggactcagcagctccactctgcctgatgaccacagcagctacacagttccaggctacatgcaggacttggaggtggagcaggccctgactccagcactgtcgccatgtgctgtcagcagcactctccccgactggcacatcccagtggaagttgtgccggacagcaccagtgatctgtacaacttccaggtgtcacccatgccctccacctctgaagctacaacagatgaggatgaggaagggaaattacctgaggacatcatgaagctcttggagcagtcggagtggcagccaacaaacgtggatgggaaggggtacctactcaatgaacctggagtccagcccacctctgtctatggagactttagctgtaaggaggagccagaaattgacagcccagggggggatattgggctgagtctacagcgtgtcttcacagatctgaagaacatggatgccacctggctggacagcctgctgaccccagtccggttgccctccatccaggccattccctgtgcaccgtag

SEQ ID NO: 3 - RNA, 558 nt, Homo sapiens
ggggauuccagcccugauaccuucucugauggacucagcagcuccacucugccugaugaccacagcagcuacacaguuccaggcuacaugcaggacuuggagguggagcaggcccugacuccagcacugucgccaugugcugucagcagcacucuccccgacuggcacaucccaguggaaguugugccggacagcaccagugaucuguacaacuuccaggugucacccaugcccuccaccucugaagcuacaacagaugaggaugaggaagggaaauuaccugaggacaucaugaagcucuuggagcagucggaguggcagccaacaaacguggaugggaagggguaccuacucaaugaaccuggaguccagcccaccucugucuauggagacuuuagcuguaaggaggagccagaaauugacagcccagggggggauauugggcugagucuacagcgugucuucacagaucugaagaacauggaugccaccuggcuggacagccugcugaccccaguccgguugcccuccauccaggccauucccugugcaccguag

SEQ ID NO: 4 - DNA, 561 bp, Artificial Sequence
  Codon optimised DNA encoding IRF1
atgggcgatagcagccccgataccttttccgatggcctgagcagcagcaccctgcctgatgatcacagcagctacaccgtgcctggctacatgcaggacctggaagtggaacaggccctgacaccagctctgagcccttgtgctgtgtccagcacactgcccgattggcacatccctgtggaagtggtgcctgacagcaccagcgacctgtacaacttccaagtgtcccctatgcctagcacctccgaggccaccaccgatgaggatgaagagggaaagctgcccgaggacatcatgaagctgctggaacagagcgagtggcagcccaccaatgtggatggcaagggctacctgctgaacgagcctggcgttcagcctacaagcgtgtacggcgacttcagctgcaaagaggaacccgagatcgatagccctggcggcgatatcggactgagcctgcagagagtgttcaccgacctgaagaacatggacgccacctggctggacagcctgctgacacctgttagactgccctctatccaggctatcccctgcgctccttga

SEQ ID NO: 5 - RNA, 561 nt, Artificial Sequence
  Codon optimised RNA encoding IRF4
augggcgauagcagccccgauaccuuuuccgauggccugagcagcagcacccugccugaugaucacagcagcuacaccgugccuggcuacaugcaggaccuggaaguggaacaggcccugacaccagcucugagcccuugugcuguguccagcacacugcccgauuggcacaucccuguggaaguggugccugacagcaccagcgaccuguacaacuuccaaguguccccuaugccuagcaccuccgaggccaccaccgaugaggaugaagagggaaagcugcccgaggacaucaugaagcugcuggaacagagcgaguggcagcccaccaauguggauggcaagggcuaccugcugaacgagccuggcguucagccuacaagcguguacggcgacuucagcugcaaagaggaacccgagaucgauagcccuggcggcgauaucggacugagccugcagagaguguucaccgaccugaagaacauggacgccaccuggcuggacagccugcugacaccuguuagacugcccucuauccaggcuauccccugcgcuccuuga

SEQ ID NO: 6 - protein, 185 aa, Homo sapiens
GDSSPDTFSDGLSSSTLPDDHSSYTVPGYMQDLEVEQALTPALSPCAVSSTLPDWHIPVEVVPDSTSDLYNFQVSPMPSTSEATTDEDEEGKLPEDIMKLLEQSEWQPTNVDGKGYLLNEPGVQPTSVYGDFSCREEPEIDSPGGDIGLSLQRVFTDLRNMDATWLDSLLTPVRLPSIQAIPCAP

SEQ ID NO: 7 - DNA, 555 bp, Homo sapiens
ggggattccagccctgataccttctctgatggactcagcagctccactctgcctgatgaccacagcagctacacagttccaggctacatgcaggacttggaggtggagcaggccctgactccagcactgtcgccatgtgctgtcagcagcactctccccgactggcacatcccagtggaagttgtgccggacagcaccagtgatctgtacaacttccaggtgtcacccatgccctccacctctgaagctacaacagatgaggatgaggaagggaaattacctgaggacatcatgaagctcttggagcagtcggagtggcagccaacaaacgtggatgggaaggggtacctactcaatgaacctggagtccagcccacctctgtctatggagactttagctgtcgggaggagccagaaattgacagcccagggggggatattgggctgagtctacagcgtgtcttcacagatctgcggaacatggatgccacctggctggacagcctgctgaccccagtccggttgccctccatccaggccattccctgtgcaccg

SEQ ID NO: 8 - RNA, 555 nt, Homo sapiens
ggggauuccagcccugauaccuucucugauggacucagcagcuccacucugccugaugaccacagcagcuacacaguuccaggcuacaugcaggacuuggagguggagcaggcccugacuccagcacugucgccaugugcugucagcagcacucuccccgacuggcacaucccaguggaaguugugccggacagcaccagugaucuguacaacuuccaggugucacccaugcccuccaccucugaagcuacaacagaugaggaugaggaagggaaauuaccugaggacaucaugaagcucuuggagcagucggaguggcagccaacaaacguggaugggaagggguaccuacucaaugaaccuggaguccagcccaccucugucuauggagacuuuagcugucgggaggagccagaaauugacagcccagggggggauauugggcugagucuacagcgugucuucacagaucugcggaacauggaugccaccuggcuggacagccugcugaccccaguccgguugcccuccauccaggccauucccugugcaccg

SEQ ID NO: 9 - DNA, 561 bp, Artificial Sequence
  Codon optimised DNA encoding IRF1
atgggcgatagcagccccgataccttttccgatggcctgagcagcagcaccctgcctgatgatcacagcagctacaccgtgcctggctacatgcaggacctggaagtggaacaggccctgacaccagctctgagcccttgtgctgtgtccagcacactgcccgattggcacatccctgtggaagtggtgcctgacagcaccagcgacctgtacaacttccaagtgtcccctatgcctagcacctccgaggccaccaccgatgaggatgaagagggaaagctgcccgaggacatcatgaagctgctggaacagagcgagtggcagcccaccaatgtggatggcaagggctacctgctgaacgagcctggcgttcagcctacaagcgtgtacggcgacttcagctgcagagaggaacccgagatcgatagccctggcggcgatatcggactgagtctgcagagggtgttcaccgacctgagaaacatggacgccacctggctggacagcctgctgacacctgttagactgccctctatccaggctatcccctgcgctccttga

SEQ ID NO: 10 - RNA, 561 nt, Artificial Sequence
  Codon optimised RNA encoding IRF1
augggcgauagcagccccgauaccuuuuccgauggccugagcagcagcacccugccugaugaucacagcagcuacaccgugccuggcuacaugcaggaccuggaaguggaacaggcccugacaccagcucugagcccuugugcuguguccagcacacugcccgauuggcacaucccuguggaaguggugccugacagcaccagcgaccuguacaacuuccaaguguccccuaugccuagcaccuccgaggccaccaccgaugaggaugaagagggaaagcugcccgaggacaucaugaagcugcuggaacagagcgaguggcagcccaccaauguggauggcaagggcuaccugcugaacgagccuggcguucagccuacaagcguguacggcgacuucagcugcagagaggaacccgagaucgauagcccuggcggcgauaucggacugagucugcagaggguguucaccgaccugagaaacauggacgccaccuggcuggacagccugcugacaccuguuagacugcccucuauccaggcuauccccugcgcuccuuga

SEQ ID NO: 11 - protein, 237 aa, Homo sapiens
PLKRLLVPGEEWEFEVTAFYRGRQVFQQTISCPEGLRLVGSEVGDRTLPGWPVTLPDPGMSLTDRGVMSYVRHVLSCLGGGLALWRAGQWLWAQRLGHCHTYWAVSEELLPNSGHGPDGEVPKDKEGGVFDLGPFIVDLITFTEGSGRSPRYALWFCVGESWPQDQPWTKRLVMVKVVPTCLRALVEMARVGGASSLENTVDLHISNSHPLSLTSDQYKAYLQDLVEGMDFQGPGES

SEQ ID NO: 12 - DNA, 711 bp, Homo sapiens
ccactgaagcggctgttggtgccgggggaagagtgggagttcgaggtgacagccttctaccggggccgccaagtcttccagcagaccatctcctgcccggagggcctgcggctggtggggtccgaagtgggagacaggacgctgcctggatggccagtcacactgccagaccctggcatgtccctgacagacaggggagtgatgagctacgtgaggcatgtgctgagctgcctgggtgggggactggctctctggcgggccgggcagtggctctgggcccagcggctggggcactgccacacatactgggcagtgagcgaggagctgctccccaacagcgggcatgggcctgatggcgaggtccccaaggacaaggaaggaggcgtgtttgacctggggcccttcattgtagatctgattaccttcacggaaggaagcggacgctcaccacgctatgccctctggttctgtgtgggggagtcatggccccaggaccagccgtggaccaagaggctcgtgatggtcaaggttgtgcccacgtgcctcagggccttggtagaaatggcccgggtagggggtgcctcctccctggagaatactgtggacctgcacatttccaacagccacccactctccctcacctccgaccagtacaaggcctacctgcaggacttggtggagggcatggatttccagggccctggggagagc

SEQ ID NO: 13 - RNA, 711 nt, Homo sapiens
ccacugaagcggcuguuggugccgggggaagagugggaguucgaggugacagccuucuaccggggccgccaagucuuccagcagaccaucuccugcccggagggccugcggcuggugggguccgaagugggagacaggacgcugccuggauggccagucacacugccagacccuggcaugucccugacagacaggggagugaugagcuacgugaggcaugugcugagcugccugggugggggacuggcucucuggcgggccgggcaguggcucugggcccagcggcuggggcacugccacacauacugggcagugagcgaggagcugcuccccaacagcgggcaugggccugauggcgagguccccaaggacaaggaaggaggcguguuugaccuggggcccuucauuguagaucugauuaccuucacggaaggaagcggacgcucaccacgcuaugcccucugguucugugugggggagucauggccccaggaccagccguggaccaagaggcucgugauggucaagguugugcccacgugccucagggccuugguagaaauggcccggguagggggugccuccucccuggagaauacuguggaccugcacauuuccaacagccacccacucucccucaccuccgaccaguacaaggccuaccugcaggacuugguggagggcauggauuuccagggcccuggggagagc

SEQ ID NO: 14 - DNA, 717 bp, Artificial Sequence
  Codon optimised DNA encoding IRF3
atgcccctgaagagactgctggtgcctggcgaggaatgggagtttgaagtgaccgccttctaccggggcagacaggtgttccagcagaccatctcttgccccgagggactgagactcgtgggctctgaagtgggcgatagaacactgcctggctggcctgtgacactgccagatcctggaatgagcctgaccgacagaggcgtgatgagctatgtgcggcacgtgctgtcttgtctcggcggaggacttgccctttggagagctggacaatggctgtgggctcagagactgggccactgtcacacatactgggccgtgtctgaggaactgctgcccaattctggccacggacctgatggcgaggtgcccaaagacaaagaaggcggcgttttcgatctgggccccttcatcgtggacctgatcacctttaccgaaggcagcggcagaagccccagatacgccctgtggttttgtgtgggcgagagctggcctcaggatcagccttggaccaagagactggtcatggtcaaggtggtgcctacctgcctgagagccctggtggaaatggctagagttggcggagccagcagcctggaaaacaccgtggatctgcacatcagcaactctcaccctctgtctctgaccagcgaccagtacaaggcctatctgcaggacctggtcgaaggcatggactttcaaggccctggcgagtcctga

SEQ ID NO: 15 - RNA, 717 nt, Artificial Sequence
  Codon optimised RNA encoding IRF3
augccccugaagagacugcuggugccuggcgaggaaugggaguuugaagugaccgccuucuaccggggcagacagguguuccagcagaccaucucuugccccgagggacugagacucgugggcucugaagugggcgauagaacacugccuggcuggccugugacacugccagauccuggaaugagccugaccgacagaggcgugaugagcuaugugcggcacgugcugucuugucucggcggaggacuugcccuuuggagagcuggacaauggcugugggcucagagacugggccacugucacacauacugggccgugucugaggaacugcugcccaauucuggccacggaccugauggcgaggugcccaaagacaaagaaggcggcguuuucgaucugggccccuucaucguggaccugaucaccuuuaccgaaggcagcggcagaagccccagauacgcccugugguuuugugugggcgagagcuggccucaggaucagccuuggaccaagagacuggucauggucaagguggugccuaccugccugagagcccugguggaaauggcuagaguuggcggagccagcagccuggaaaacaccguggaucugcacaucagcaacucucacccucugucucugaccagcgaccaguacaaggccuaucugcaggaccuggucgaaggcauggacuuucaaggcccuggcgaguccuga

SEQ ID NO: 16 - protein, 266 aa, Homo sapiens
WAVETTPSPGPQPAALTTGEAAAPESPHQAEPYLSPSPSACTAVQEPSPGALDVTIMYKGRTVLQKVVGHPSCTFLYGPPDPAVRATDPQQVAFPSPAELPDQKQLRYTEELLRHVAPGLHLELRGPQLWARRMGKCKVYWEVGGPPGSASPSTPACLLPRNCDTPIFDFRVFFQELVEFRARQRRGSPRYTIYLGFGQDLSAGRPKEKSLVLVKLEPWLCRVHLEGTQREGVSSLDSSSLSLCLSSANSLYDDIECFLMELEQPA

SEQ ID NO: 17 - DNA, 798 bp, Homo sapiens
tgggcagtagagacgacccccagccccgggccccagcccgcggcactaacgacaggcgaggccgcggccccagagtccccgcaccaggcagagccgtacctgtcaccctccccaagcgcctgcaccgcggtgcaagagcccagcccaggggcgctggacgtgaccatcatgtacaagggccgcacggtgctgcagaaggtggtgggacacccgagctgcacgttcctatacggccccccagacccagctgtccgggccacagacccccagcaggtagcattccccagccctgccgagctcccggaccagaagcagctgcgctacacggaggaactgctgcggcacgtggcccctgggttgcacctggagcttcgggggccacagctgtgggcccggcgcatgggcaagtgcaaggtgtactgggaggtgggcggacccccaggctccgccagcccctccaccccagcctgcctgctgcctcggaactgtgacacccccatcttcgacttcagagtcttcttccaagagctggtggaattccgggcacggcagcgccgtggctccccacgctataccatctacctgggcttcgggcaggacctgtcagctgggaggcccaaggagaagagcctggtcctggtgaagctggaaccctggctgtgccgagtgcacctagagggcacgcagcgtgagggtgtgtcttccctggatagcagcagcctcagcctctgcctgtccagcgccaacagcctctatgacgacatcgagtgcttccttatggagctggagcagcccgcc

SEQ ID NO: 18 - RNA, 798 nt, Homo sapiens
ugggcaguagagacgacccccagccccgggccccagcccgcggcacuaacgacaggcgaggccgcggccccagaguccccgcaccaggcagagccguaccugucacccuccccaagcgccugcaccgcggugcaagagcccagcccaggggcgcuggacgugaccaucauguacaagggccgcacggugcugcagaagguggugggacacccgagcugcacguuccuauacggccccccagacccagcuguccgggccacagacccccagcagguagcauuccccagcccugccgagcucccggaccagaagcagcugcgcuacacggaggaacugcugcggcacguggccccuggguugcaccuggagcuucgggggccacagcugugggcccggcgcaugggcaagugcaagguguacugggaggugggcggacccccaggcuccgccagccccuccaccccagccugccugcugccucggaacugugacacccccaucuucgacuucagagucuucuuccaagagcugguggaauuccgggcacggcagcgccguggcuccccacgcuauaccaucuaccugggcuucgggcaggaccugucagcugggaggcccaaggagaagagccugguccuggugaagcuggaacccuggcugugccgagugcaccuagagggcacgcagcgugagggugugucuucccuggauagcagcagccucagccucugccuguccagcgccaacagccucuaugacgacaucgagugcuuccuuauggagcuggagcagcccgcc

SEQ ID NO: 19 - DNA, 804 bp, Artificial Sequence
  Codon optimised DNA encoding IRF7
atgtgggccgtcgagacaacaccttctccaggacctcaacctgccgctctgacaacaggcgaagctgctgctcctgagtctccacatcaggccgagccttacctgtctccatctcctagcgcctgtaccgccgtgcaagaaccttctcctggtgctctggacgtgaccatcatgtacaagggcagaaccgtgctgcagaaagtcgtgggacaccccagctgcacctttctgtatggccctccagatcctgccgtgcgggctacagatcctcagcaggttgcattcccatctccagccgagctgcccgatcagaagcagctgagatacaccgaggaactgctgagacacgtggcccctggactgcacctggaactgagaggaccacaactgtgggccagacggatgggcaagtgcaaggtgtactgggaagttggcggccctcctggatctgcctctccatctacaccagcctgcctgctgcctcggaattgcgacacccctatcttcgacttccgggtgttcttccaagagctggtggaattccgggccagacagagaagaggcagccccagatacaccatctacctcggctttggccaggacctgtctgccggacggcctaaagaaaagtccctggtgctggtcaagctggaaccctggctgtgtagagtgcatctggaaggcacccagagagagggcgtcagcagcctggatagcagctctctgagcctgtgtctgagcagcgccaacagcctgtacgacgatatcgagtgcttcctgatggaactggaacagcccgcctga

SEQ ID NO: 20 - RNA, 804 nt, Artificial Sequence
  Codon optimised RNA encoding IRF7
augugggccgucgagacaacaccuucuccaggaccucaaccugccgcucugacaacaggcgaagcugcugcuccugagucuccacaucaggccgagccuuaccugucuccaucuccuagcgccuguaccgccgugcaagaaccuucuccuggugcucuggacgugaccaucauguacaagggcagaaccgugcugcagaaagucgugggacaccccagcugcaccuuucuguauggcccuccagauccugccgugcgggcuacagauccucagcagguugcauucccaucuccagccgagcugcccgaucagaagcagcugagauacaccgaggaacugcugagacacguggccccuggacugcaccuggaacugagaggaccacaacugugggccagacggaugggcaagugcaagguguacugggaaguuggcggcccuccuggaucugccucuccaucuacaccagccugccugcugccucggaauugcgacaccccuaucuucgacuuccggguguucuuccaagagcugguggaauuccgggccagacagagaagaggcagccccagauacaccaucuaccucggcuuuggccaggaccugucugccggacggccuaaagaaaagucccuggugcuggucaagcuggaacccuggcuguguagagugcaucuggaaggcacccagagagagggcgucagcagccuggauagcagcucucugagccugugucugagcagcgccaacagccuguacgacgauaucgagugcuuccugauggaacuggaacagcccgccuga

SEQ ID NO: 21 - protein, 251 aa, Homo sapiens
KEEEDAMQNCTLSPSVLQDSLNNEEEGASGGAVHSDIGSSSSSSSPEPQEVTDTTEAPFQGDQRSLEFLLPPEPDYSLLLTFIYNGRVVGEAQVQSLDCRLVAEPSGSESSMEQVLFPKPGPLEPTQRLLSQLERGILVASNPRGLFVQRLCPIPISWNAPQAPPGPGPHLLPSNECVELFRTAYFCRDLVRYFQGLGPPPKFQVTLNFWEESHGSSHTPQNLITVKMEQAFARYLLEQTPEQQAAILSLV

SEQ ID NO: 22 - DNA, 753 bp, Homo sapiens
aaggaggaagaggatgccatgcagaactgcacactcagtccctctgtgctccaggactccctcaataatgaggaggagggggccagtgggggagcagtccattcagacattgggagcagcagcagcagcagcagccctgagccacaggaagttacagacacaactgaggccccctttcaaggggatcagaggtccctggagtttctgcttcctccagagccagactactcactgctgctcaccttcatctacaacgggcgcgtggtgggcgaggcccaggtgcaaagcctggattgccgccttgtggctgagccctcaggctctgagagcagcatggagcaggtgctgttccccaagcctggcccactggagcccacgcagcgcctgctgagccagcttgagaggggcatcctagtggccagcaacccccgaggcctcttcgtgcagcgcctttgccccatccccatctcctggaatgcaccccaggctccacctgggccaggcccgcatctgctgcccagcaacgagtgcgtggagctcttcagaaccgcctacttctgcagagacttggtcaggtactttcagggcctgggccccccaccgaagttccaggtaacactgaatttctgggaagagagccatggctccagccatactccacagaatcttatcacagtgaagatggagcaggcctttgcccgatacttgctggagcagactccagagcagcaggcagccattctgtccctggtg

SEQ ID NO: 23 - RNA, 753 nt, Homo sapiens
aaggaggaagaggaugccaugcagaacugcacacucagucccucugugcuccaggacucccucaauaaugaggaggagggggccagugggggagcaguccauucagacauugggagcagcagcagcagcagcagcccugagccacaggaaguuacagacacaacugaggcccccuuucaaggggaucagaggucccuggaguuucugcuuccuccagagccagacuacucacugcugcucaccuucaucuacaacgggcgcguggugggcgaggcccaggugcaaagccuggauugccgccuuguggcugagcccucaggcucugagagcagcauggagcaggugcuguuccccaagccuggcccacuggagcccacgcagcgccugcugagccagcuugagaggggcauccuaguggccagcaacccccgaggccucuucgugcagcgccuuugccccauccccaucuccuggaaugcaccccaggcuccaccugggccaggcccgcaucugcugcccagcaacgagugcguggagcucuucagaaccgccuacuucugcagagacuuggucagguacuuucagggccugggccccccaccgaaguuccagguaacacugaauuucugggaagagagccauggcuccagccauacuccacagaaucuuaucacagugaagauggagcaggccuuugcccgauacuugcuggagcagacuccagagcagcaggcagccauucugucccuggug

SEQ ID NO: 24 - DNA, 759 bp, Artificial Sequence
  Codon optimised DNA encoding IRF9
atgaaggaagaagaggacgccatgcagaactgcacactgagcccaagcgtgctgcaggacagcctgaacaatgaggaagaaggcgcctctggcggagccgtgcactctgatattggcagcagcagctctagcagcagccccgagcctcaagaagtgaccgatacaacagaggccccattccagggcgaccagcggagtctggaatttctgctgcctcctgagcctgactacagcctgctgctgaccttcatctacaacggcagagtcgtgggcgaagcccaggtgcagtctctggattgcagactggtggccgagcctagcggaagcgagtctagtatggaacaggtgctgttccccaagcctggacctctggaacccacacagaggctgctgtctcaactggaaaggggcatcctggtggccagcaatcctagaggcctgttcgtgcagagactgtgccctattcctatcagctggaacgcccctcaggctcctcctggacctggaccacatctgctgcccagcaatgagtgcgtggaactgttccggaccgcctacttctgcagagatctcgtgcggtacttccaaggcctgggacctcctccaaagttccaagtgaccctgaacttctgggaagagagccacggcagcagccacacacctcagaatctgatcaccgtgaagatggaacaagccttcgccagatacctgctggaacagacccctgaacagcaggccgccatcctgtctctggtgtga

SEQ ID NO: 25 - RNA, 759 nt, Artificial Sequence
  Codon optimised RNA encoding IRF9
augaaggaagaagaggacgccaugcagaacugcacacugagcccaagcgugcugcaggacagccugaacaaugaggaagaaggcgccucuggcggagccgugcacucugauauuggcagcagcagcucuagcagcagccccgagccucaagaagugaccgauacaacagaggccccauuccagggcgaccagcggagucuggaauuucugcugccuccugagccugacuacagccugcugcugaccuucaucuacaacggcagagucgugggcgaagcccaggugcagucucuggauugcagacugguggccgagccuagcggaagcgagucuaguauggaacaggugcuguuccccaagccuggaccucuggaacccacacagaggcugcugucucaacuggaaaggggcauccugguggccagcaauccuagaggccuguucgugcagagacugugcccuauuccuaucagcuggaacgccccucaggcuccuccuggaccuggaccacaucugcugcccagcaaugagugcguggaacuguuccggaccgccuacuucugcagagaucucgugcgguacuuccaaggccugggaccuccuccaaaguuccaagugacccugaacuucugggaagagagccacggcagcagccacacaccucagaaucugaucaccgugaagauggaacaagccuucgccagauaccugcuggaacagaccccugaacagcaggccgccauccugucucugguguga

SEQ ID NO: 26 - protein, 204 aa, Homo sapiens
SSSSSSPEPQEVTDTTEAPFQGDQRSLEFLLPPEPDYSLLLTFIYNGRVVGEAQVQSLDCRLVAEPSGSESSMEQVLFPKPGPLEPTQRLLSQLERGILVASNPRGLFVQRLCPIPISWNAPQAPPGPGPHLLPSNECVELFRTAYFCRDLVRYFQGLGPPPKFQVTLNFWEESHGSSHTPQNLITVKMEQAFARYLLEQTPEQ

SEQ ID NO: 27 - DNA, 612 bp, Homo sapiens
agcagcagcagcagcagccctgagccacaggaagttacagacacaactgaggccccctttcaaggggatcagaggtccctggagtttctgcttcctccagagccagactactcactgctgctcaccttcatctacaacgggcgcgtggtgggcgaggcccaggtgcaaagcctggattgccgccttgtggctgagccctcaggctctgagagcagcatggagcaggtgctgttccccaagcctggcccactggagcccacgcagcgcctgctgagccagcttgagaggggcatcctagtggccagcaacccccgaggcctcttcgtgcagcgcctttgccccatccccatctcctggaatgcaccccaggctccacctgggccaggcccgcatctgctgcccagcaacgagtgcgtggagctcttcagaaccgcctacttctgcagagacttggtcaggtactttcagggcctgggccccccaccgaagttccaggtaacactgaatttctgggaagagagccatggctccagccatactccacagaatcttatcacagtgaagatggagcaggcctttgcccgatacttgctggagcagactccagagcag

SEQ ID NO: 28 - RNA, 612 nt, Homo sapiens
agcagcagcagcagcagcccugagccacaggaaguuacagacacaacugaggcccccuuucaaggggaucagaggucccuggaguuucugcuuccuccagagccagacuacucacugcugcucaccuucaucuacaacgggcgcguggugggcgaggcccaggugcaaagccuggauugccgccuuguggcugagcccucaggcucugagagcagcauggagcaggugcuguuccccaagccuggcccacuggagcccacgcagcgccugcugagccagcuugagaggggcauccuaguggccagcaacccccgaggccucuucgugcagcgccuuugccccauccccaucuccuggaaugcaccccaggcuccaccugggccaggcccgcaucugcugcccagcaacgagugcguggagcucuucagaaccgccuacuucugcagagacuuggucagguacuuucagggccugggccccccaccgaaguuccagguaacacugaauuucugggaagagagccauggcuccagccauacuccacagaaucuuaucacagugaagauggagcaggccuuugcccgauacuugcuggagcagacuccagagcag

SEQ ID NO: 29 - DNA, 618 bp, Artificial Sequence
  Codon optimised DNA encoding IRF9
atgagcagctctagcagcagccccgagcctcaagaagtgaccgatacaacagaggccccattccagggcgaccagcggagtctggaatttctgctgcctcctgagcctgactacagcctgctgctgaccttcatctacaacggcagagtcgtgggcgaagcccaggtgcagtctctggattgcagactggtggccgagcctagcggaagcgagtctagtatggaacaggtgctgttccccaagcctggacctctggaacccacacagaggctgctgtctcaactggaaaggggcatcctggtggccagcaatcctagaggcctgttcgtgcagagactgtgccctattcctatcagctggaacgcccctcaggctcctcctggacctggaccacatctgctgcccagcaatgagtgcgtggaactgttccggaccgcctacttctgcagagatctcgtgcggtacttccaaggcctgggacctcctccaaagttccaagtgaccctgaacttctgggaagagagccacggcagcagccacacacctcagaatctgatcaccgtgaagatggaacaagccttcgccagatacctgctggaacagacccctgaacagtga

SEQ ID NO: 30 - RNA, 618 nt, Artificial Sequence
  Codon optimised RNA encoding IRF9
augagcagcucuagcagcagccccgagccucaagaagugaccgauacaacagaggccccauuccagggcgaccagcggagucuggaauuucugcugccuccugagccugacuacagccugcugcugaccuucaucuacaacggcagagucgugggcgaagcccaggugcagucucuggauugcagacugguggccgagccuagcggaagcgagucuaguauggaacaggugcuguuccccaagccuggaccucuggaacccacacagaggcugcugucucaacuggaaaggggcauccugguggccagcaauccuagaggccuguucgugcagagacugugcccuauuccuaucagcuggaacgccccucaggcuccuccuggaccuggaccacaucugcugcccagcaaugagugcguggaacuguuccggaccgccuacuucugcagagaucucgugcgguacuuccaaggccugggaccuccuccaaaguuccaagugacccugaacuucugggaagagagccacggcagcagccacacaccucagaaucugaucaccgugaagauggaacaagccuucgccagauaccugcuggaacagaccccugaacaguga

SEQ ID NO: 31 - protein, 109 aa, Homo sapiens
PFQGDQRSLEFLLPPEPDYSLLLTFIYNGRVVGEAQVQSLDCRLVAEPSGSESSMEQVLFPKPGPLEPTQRLLSQLERGILVASNPRGLFVQRLCPIPISWNAPQAPPG

SEQ ID NO: 32 - DNA, 327 bp, Homo sapiens
ccctttcaaggggatcagaggtccctggagtttctgcttcctccagagccagactactcactgctgctcaccttcatctacaacgggcgcgtggtgggcgaggcccaggtgcaaagcctggattgccgccttgtggctgagccctcaggctctgagagcagcatggagcaggtgctgttccccaagcctggcccactggagcccacgcagcgcctgctgagccagcttgagaggggcatcctagtggccagcaacccccgaggcctcttcgtgcagcgcctttgccccatccccatctcctggaatgcaccccaggctccacctggg

SEQ ID NO: 33 - RNA, 327 nt, Homo sapiens
cccuuucaaggggaucagaggucccuggaguuucugcuuccuccagagccagacuacucacugcugcucaccuucaucuacaacgggcgcguggugggcgaggcccaggugcaaagccuggauugccgccuuguggcugagcccucaggcucugagagcagcauggagcaggugcuguuccccaagccuggcccacuggagcccacgcagcgccugcugagccagcuugagaggggcauccuaguggccagcaacccccgaggccucuucgugcagcgccuuugccccauccccaucuccuggaaugcaccccaggcuccaccuggg

SEQ ID NO: 34 - DNA, 333 bp, Artificial Sequence
  Codon optimised DNA encoding IRF9
atgccattccagggcgaccagcggagtctggaatttctgctgcctcctgagcctgactacagcctgctgctgaccttcatctacaacggcagagtcgtgggcgaagcccaggtgcagtctctggattgcagactggtggccgagcctagcggaagcgagtctagtatggaacaggtgctgttccccaagcctggacctctggaacccacacagaggctgctgtctcaactggaaaggggcatcctggtggccagcaatcctagaggcctgttcgtgcagagactgtgccctattcctatcagctggaacgcccctcaggctcctcctggatga

SEQ ID NO: 35 - RNA, 333 nt, Artificial Sequence
  Codon optimised RNA encoding IRF9
augccauuccagggcgaccagcggagucuggaauuucugcugccuccugagccugacuacagccugcugcugaccuucaucuacaacggcagagucgugggcgaagcccaggugcagucucuggauugcagacugguggccgagccuagcggaagcgagucuaguauggaacaggugcuguuccccaagccuggaccucuggaacccacacagaggcugcugucucaacuggaaaggggcauccugguggccagcaauccuagaggccuguucgugcagagacugugcccuauuccuaucagcuggaacgccccucaggcuccuccuggauga

SEQ ID NO: 36 - protein, 164 aa, Homo sapiens
MPITRMRMRPWLEMQINSNQIPGLIWINKEEMIFQIPWKHAAKHGWDINKDACLFRSWAIHTGRYKAGEKEPDPKTWKANFRCAMNSLPDIEEVKDQSRNKGSSAVRVYRMLPPLTKNQRKERKSKSSRDAKSKAKRKSCGDSSPDTFSDGLSSSTLPDDHSSY

SEQ ID NO: 37 - DNA, 492 bp, Homo sapiens
atgcccatcactcggatgcgcatgagaccctggctagagatgcagattaattccaaccaaatcccggggctcatctggattaataaagaggagatgatcttccagatcccatggaagcatgctgccaagcatggctgggacatcaacaaggatgcctgtttgttccggagctgggccattcacacaggccgatacaaagcaggggaaaaggagccagatcccaagacgtggaaggccaactttcgctgtgccatgaactccctgccagatatcgaggaggtgaaagaccagagcaggaacaagggcagctcagctgtgcgagtgtaccggatgcttccacctctcaccaagaaccagagaaaagaaagaaagtcgaagtccagccgagatgctaagagcaaggccaagaggaagtcatgtggggattccagccctgataccttctctgatggactcagcagctccactctgcctgatgaccacagcagctac

SEQ ID NO: 38 - RNA, 492 nt, Homo sapiens
augcccaucacucggaugcgcaugagacccuggcuagagaugcagauuaauuccaaccaaaucccggggcucaucuggauuaauaaagaggagaugaucuuccagaucccauggaagcaugcugccaagcauggcugggacaucaacaaggaugccuguuuguuccggagcugggccauucacacaggccgauacaaagcaggggaaaaggagccagaucccaagacguggaaggccaacuuucgcugugccaugaacucccugccagauaucgaggaggugaaagaccagagcaggaacaagggcagcucagcugugcgaguguaccggaugcuuccaccucucaccaagaaccagagaaaagaaagaaagucgaaguccagccgagaugcuaagagcaaggccaagaggaagucauguggggauuccagcccugauaccuucucugauggacucagcagcuccacucugccugaugaccacagcagcuac

SEQ ID NO: 39 - DNA, 495 bp, Artificial Sequence
  Codon optimised DNA encoding IRF1
atgcccatcaccagaatgagaatgcggccctggctggaaatgcagatcaacagcaatcagatccccggcctgatctggatcaacaaagaagagatgatctttcagatcccgtggaagcacgccgccaagcacggatgggacatcaacaaggacgcctgcctgttcagaagctgggccatccacaccggcagatacaaggccggcgagaaagagcccgatcctaagacctggaaggccaacttcagatgcgccatgaacagcctgcctgacatcgaggaagtgaaggaccagagccggaacaagggatcttctgccgtgcgggtgtaccggatgttgcctcctctgaccaagaaccagcgcaaagagcggaagtccaagagcagcagagatgccaagagcaaggccaagagaaagtcctgcggcgacagcagccctgacaccttttctgatggcctgagcagcagcaccctgccagatgatcacagcagctactga

SEQ ID NO: 40 - RNA, 495 nt, Artificial Sequence
  Codon optimised RNA encoding IRF1
augcccaucaccagaaugagaaugcggcccuggcuggaaaugcagaucaacagcaaucagauccccggccugaucuggaucaacaaagaagagaugaucuuucagaucccguggaagcacgccgccaagcacggaugggacaucaacaaggacgccugccuguucagaagcugggccauccacaccggcagauacaaggccggcgagaaagagcccgauccuaagaccuggaaggccaacuucagaugcgccaugaacagccugccugacaucgaggaagugaaggaccagagccggaacaagggaucuucugccgugcggguguaccggauguugccuccucugaccaagaaccagcgcaaagagcggaaguccaagagcagcagagaugccaagagcaaggccaagagaaaguccugcggcgacagcagcccugacaccuuuucugauggccugagcagcagcacccugccagaugaucacagcagcuacuga

SEQ ID NO: 41 - protein, 109 aa, Homo sapiens
NGKLRQWLIDQIDSGKYPGLVWENEEKSIFRIPWKHAGKQDYNREEDAALFKAWALFKGKFREGIDKPDPPTWKTRLRCALNKSNDFEELVERSQLDISDPYKVYRIVP

SEQ ID NO: 42 - DNA, 327 bp, Homo sapiens
aacgggaagctccgccagtggctgatcgaccagatcgacagcggcaagtaccccgggctggtgtgggagaacgaggagaagagcatcttccgcatcccctggaagcacgcgggcaagcaggactacaaccgcgaggaggacgccgcgctcttcaaggcttgggcactgtttaaaggaaagttccgagaaggcatcgacaagccggaccctcccacctggaagacgcgcctgcggtgcgctttgaacaagagcaatgactttgaggaactggttgagcggagccagctggacatctcagacccgtacaaagtgtacaggattgttcct

SEQ ID NO: 43 - RNA, 327 nt, Homo sapiens
aacgggaagcuccgccaguggcugaucgaccagaucgacagcggcaaguaccccgggcuggugugggagaacgaggagaagagcaucuuccgcauccccuggaagcacgcgggcaagcaggacuacaaccgcgaggaggacgccgcgcucuucaaggcuugggcacuguuuaaaggaaaguuccgagaaggcaucgacaagccggacccucccaccuggaagacgcgccugcggugcgcuuugaacaagagcaaugacuuugaggaacugguugagcggagccagcuggacaucucagacccguacaaaguguacaggauuguuccu

SEQ ID NO: 44 - DNA, 333 bp, Artificial Sequence
  Codon optimised DNA encoding IRF4
atgaacggcaagctgcggcagtggctgatcgaccagatcgacagcggcaagtatcctggcctcgtgtgggagaacgaggaaaagtctatcttcagaatcccctggaagcacgccggcaagcaggactacaacagagaagaggacgccgctctgttcaaggcctgggctctgtttaagggcaagttcagagagggcatcgacaagcccgatcctccaacctggaaaaccagactgagatgcgccctgaacaagagcaacgacttcgaggaactggtggaaagaagccagctggacatcagcgacccctacaaggtgtaccggatcgtgccttga

SEQ ID NO: 45 - RNA, 333 nt, Artificial Sequence
  Codon optimised RNA encoding IRF4
augaacggcaagcugcggcaguggcugaucgaccagaucgacagcggcaaguauccuggccucgugugggagaacgaggaaaagucuaucuucagaauccccuggaagcacgccggcaagcaggacuacaacagagaagaggacgccgcucuguucaaggccugggcucuguuuaagggcaaguucagagagggcaucgacaagcccgauccuccaaccuggaaaaccagacugagaugcgcccugaacaagagcaacgacuucgaggaacugguggaaagaagccagcuggacaucagcgaccccuacaagguguaccggaucgugccuuga

SEQ ID NO: 46 - protein, 140 aa, Homo sapiens
MNQSIPVAPTPPRRVRLKPWLVAQVNSCQYPGLQWVNGEKKLFCIPWRHATRHGPSQDGDNTIFKAWAKETGKYTEGVDEADPAKWKANLRCALNKSRDFRLIYDGPRDMPPQPYKIYEVCSNGPAPTDSQPPEDYSFGA

SEQ ID NO: 47 - DNA, 420 bp, Homo sapiens
atgaaccagtccatcccagtggctcccaccccaccccgccgcgtgcggctgaagccctggctggtggcccaggtgaacagctgccagtacccagggcttcaatgggtcaacggggaaaagaaattattctgcatcccctggaggcatgccacaaggcatggtcccagccaggacggagataacaccatcttcaaggcctgggccaaggagacagggaaatacaccgaaggcgtggatgaagccgatccggccaagtggaaggccaacctgcgctgtgcccttaacaagagccgggacttccgcctcatctacgacgggccccgggacatgccacctcagccctacaagatctacgaggtctgctccaatggccctgctcccacagactcccagccccctgaggattactcttttggtgca

SEQ ID NO: 48 - RNA, 420 nt, Homo sapiens
augaaccaguccaucccaguggcucccaccccaccccgccgcgugcggcugaagcccuggcugguggcccaggugaacagcugccaguacccagggcuucaaugggucaacggggaaaagaaauuauucugcauccccuggaggcaugccacaaggcauggucccagccaggacggagauaacaccaucuucaaggccugggccaaggagacagggaaauacaccgaaggcguggaugaagccgauccggccaaguggaaggccaaccugcgcugugcccuuaacaagagccgggacuuccgccucaucuacgacgggccccgggacaugccaccucagcccuacaagaucuacgaggucugcuccaauggcccugcucccacagacucccagcccccugaggauuacucuuuuggugca

SEQ ID NO: 49 - DNA, 423 bp, Artificial Sequence
  Codon optimised DNA encoding IRF5
atgaaccagagcatccccgtggctcccacacctcctagaagagtgcgactgaagccttggctggtggcccaagtgaacagctgtcagtatcctggcctgcagtgggtcaacggcgagaagaagctgttctgcatcccttggagacacgccaccagacacggcccttctcaggacggcgacaacaccatctttaaggcctgggccaaagagacaggcaagtacaccgaaggcgtggacgaagccgatcctgccaagtggaaggccaatctgagatgcgccctgaacaagagccgggacttccggctgatctacgacggccctagagacatgcctcctcagccttacaagatctacgaagtgtgcagcaacggccctgctcctaccgattctcagcctcctgaggactacagcttcggcgcttga

SEQ ID NO: 50 - RNA, 423 nt, Artificial Sequence
  Codon optimised RNA encoding IRF5
augaaccagagcauccccguggcucccacaccuccuagaagagugcgacugaagccuuggcugguggcccaagugaacagcugucaguauccuggccugcagugggucaacggcgagaagaagcuguucugcaucccuuggagacacgccaccagacacggcccuucucaggacggcgacaacaccaucuuuaaggccugggccaaagagacaggcaaguacaccgaaggcguggacgaagccgauccugccaaguggaaggccaaucugagaugcgcccugaacaagagccgggacuuccggcugaucuacgacggcccuagagacaugccuccucagccuuacaagaucuacgaagugugcagcaacggcccugcuccuaccgauucucagccuccugaggacuacagcuucggcgcuuga

SEQ ID NO: 51 - protein, 140 aa, Homo sapiens
MNQSIPVAPTPPRRVRLKPWLVAQVNSCQYPGLQWVNGEKKLFCIPWRHATRHGPSQDGDNTIFKAWPKETGKYTEGVDEADPAKWKANLRCALNKSRDFRLIYDGPRDMPPQPYKIYEVCSNGPAPTDSQPPEDYSFGA

SEQ ID NO: 52 - DNA, 420 bp, Homo sapiens
atgaaccagtccatcccagtggctcccaccccaccccgccgcgtgcggctgaagccctggctggtggcccaggtgaacagctgccagtacccagggcttcaatgggtcaacggggaaaagaaattattctgcatcccctggaggcatgccacaaggcatggtcccagccaggacggagataacaccatcttcaaggcctggcccaaggagacagggaaatacaccgaaggcgtggatgaagccgatccggccaagtggaaggccaacctgcgctgtgcccttaacaagagccgggacttccgcctcatctacgacgggccccgggacatgccacctcagccctacaagatctacgaggtctgctccaatggccctgctcccacagactcccagccccctgaggattactcttttggtgca

SEQ ID NO: 53 - RNA, 420 nt, Homo sapiens
augaaccaguccaucccaguggcucccaccccaccccgccgcgugcggcugaagcccuggcugguggcccaggugaacagcugccaguacccagggcuucaaugggucaacggggaaaagaaauuauucugcauccccuggaggcaugccacaaggcauggucccagccaggacggagauaacaccaucuucaaggccuggcccaaggagacagggaaauacaccgaaggcguggaugaagccgauccggccaaguggaaggccaaccugcgcugugcccuuaacaagagccgggacuuccgccucaucuacgacgggccccgggacaugccaccucagcccuacaagaucuacgaggucugcuccaauggcccugcucccacagacucccagcccccugaggauuacucuuuuggugca

SEQ ID NO: 54 - DNA, 423 bp, Artificial Sequence
  Codon optimised DNA enoding IRF5
atgaaccagagcatccccgtggctcccacacctcctagaagagtgcgactgaagccttggctggtggcccaagtgaacagctgtcagtatcctggcctgcagtgggtcaacggcgagaagaagctgttctgcatcccttggagacacgccaccagacacggcccttctcaggacggcgacaacaccatctttaaggcctggcccaaagagacaggcaagtacaccgaaggcgtggacgaagccgatcctgccaagtggaaggccaatctgagatgcgccctgaacaagagccgggacttccggctgatctacgacggccctagagacatgcctcctcagccttacaagatctacgaagtgtgcagcaacggccctgctcctaccgattctcagcctcctgaggactacagcttcggcgcttga

SEQ ID NO: 55 - RNA, 423 nt, Artificial Sequence
  Codon optimised RNA encoding IRF5
augaaccagagcauccccguggcucccacaccuccuagaagagugcgacugaagccuuggcugguggcccaagugaacagcugucaguauccuggccugcagugggucaacggcgagaagaagcuguucugcaucccuuggagacacgccaccagacacggcccuucucaggacggcgacaacaccaucuuuaaggccuggcccaaagagacaggcaaguacaccgaaggcguggacgaagccgauccugccaaguggaaggccaaucugagaugcgcccugaacaagagccgggacuuccggcugaucuacgacggcccuagagacaugccuccucagccuuacaagaucuacgaagugugcagcaacggcccugcuccuaccgauucucagccuccugaggacuacagcuucggcgcuuga

SEQ ID NO: 56 - protein, 140 aa, Homo sapiens
MCDRNGGRRLRQWLIEQIDSSMYPGLIWENEEKSMFRIPWKHAGKQDYNQEVDASIFKAWAVFKGKFKEGDKAEPATWKTRLRCALNKSPDFEEVTDRSQLDISEPYKVYRIVPEEEQKCKLGVATAGCVNEVTEMECGR

SEQ ID NO: 57 - DNA, 420 bp, Homo sapiens
atgtgtgaccggaatggtggtcggcggcttcgacagtggctgatcgagcagattgacagtagcatgtatccaggactgatttgggagaatgaggagaagagcatgttccggatcccttggaaacacgctggcaagcaagattataatcaggaagtggatgcctccatttttaaggcctgggcagtttttaaagggaagtttaaagaaggggacaaagctgaaccagccacttggaagacgaggttacgctgtgctttgaataagagcccagattttgaggaagtgacggaccggtcccaactggacatttccgagccatacaaagtttaccgaattgttcctgaggaagagcaaaaatgcaaactaggcgtggcaactgctggctgcgtgaatgaagttacagagatggagtgcggtcgc

SEQ ID NO: 58 - RNA, 420 nt, Homo sapiens
augugugaccggaaugguggucggcggcuucgacaguggcugaucgagcagauugacaguagcauguauccaggacugauuugggagaaugaggagaagagcauguuccggaucccuuggaaacacgcuggcaagcaagauuauaaucaggaaguggaugccuccauuuuuaaggccugggcaguuuuuaaagggaaguuuaaagaaggggacaaagcugaaccagccacuuggaagacgagguuacgcugugcuuugaauaagagcccagauuuugaggaagugacggaccggucccaacuggacauuuccgagccauacaaaguuuaccgaauuguuccugaggaagagcaaaaaugcaaacuaggcguggcaacugcuggcugcgugaaugaaguuacagagauggagugcggucgc

SEQ ID NO: 59 - DNA, 423 bp, Artificial Sequence
  Codon optimised DNA encoding IRF8
atgtgcgacagaaatggcggcagacggctgagacagtggctgatcgagcagatcgacagcagcatgtaccccggcctgatctgggagaacgaagagaagtctatgttcaggatcccctggaagcacgccggcaagcaggactacaatcaagaggtggacgccagcatcttcaaggcctgggccgtgttcaagggcaagttcaaagagggcgacaaggccgagcctgccacctggaaaaccagactgagatgcgccctgaacaagagccccgacttcgaggaagtgaccgacagaagccagctggacatcagcgagccctacaaggtgtaccggatcgtgcccgaagaggaacagaaatgcaagctgggcgttgccaccgccggctgtgtgaatgaagtgacagagatggaatgcggccggtga

SEQ ID NO: 60 - RNA, 423 nt, Artificial Sequence
  Codon optimised RNA encoding IRF8
augugcgacagaaauggcggcagacggcugagacaguggcugaucgagcagaucgacagcagcauguaccccggccugaucugggagaacgaagagaagucuauguucaggauccccuggaagcacgccggcaagcaggacuacaaucaagagguggacgccagcaucuucaaggccugggccguguucaagggcaaguucaaagagggcgacaaggccgagccugccaccuggaaaaccagacugagaugcgcccugaacaagagccccgacuucgaggaagugaccgacagaagccagcuggacaucagcgagcccuacaagguguaccggaucgugcccgaagaggaacagaaaugcaagcugggcguugccaccgccggcugugugaaugaagugacagagauggaaugcggccgguga

SEQ ID NO: 61 - protein, 120 aa, Homo sapiens
MASGRARCTRKLRNWVVEQVESGQFPGVCWDDTAKTMFRIPWKHAGKQDFREDQDAAFFKAWAIFKGKYKEGDTGGPAVWKTRLRCALNKSSEFKEVPERGRMDVAEPYKVYQLLPPGIV

SEQ ID NO: 62 - DNA, 360 bp, Homo sapiens
atggcatcaggcagggcacgctgcacccgaaaactccggaactgggtggtggagcaagtggagagtgggcagtttcccggagtgtgctgggatgatacagctaagaccatgttccggattccctggaaacatgcaggcaagcaggacttccgggaggaccaggatgctgccttcttcaaggcctgggcaatatttaagggaaagtataaggagggggacacaggaggtccagctgtctggaagactcgcctgcgctgtgcactcaacaagagttctgaatttaaggaggttcctgagaggggccgcatggatgttgctgagccctacaaggtgtatcagttgctgccaccaggaatcgtc

SEQ ID NO: 63 - RNA, 360 nt, Homo sapiens
auggcaucaggcagggcacgcugcacccgaaaacuccggaacugggugguggagcaaguggagagugggcaguuucccggagugugcugggaugauacagcuaagaccauguuccggauucccuggaaacaugcaggcaagcaggacuuccgggaggaccaggaugcugccuucuucaaggccugggcaauauuuaagggaaaguauaaggagggggacacaggagguccagcugucuggaagacucgccugcgcugugcacucaacaagaguucugaauuuaaggagguuccugagaggggccgcauggauguugcugagcccuacaagguguaucaguugcugccaccaggaaucguc

SEQ ID NO: 64 - DNA, 363 bp, Artificial Sequence
  Codon optimised DNA encoding IRF9
atggcttctggcagagccagatgcacccggaagctgagaaactgggtcgtcgaacaggtggaaagcggacagttccctggcgtgtgctgggatgataccgccaagacaatgttcagaatcccctggaagcacgccggcaagcaggacttcagagaagatcaggacgccgccttcttcaaggcctgggccatcttcaagggcaagtacaaagagggcgacacaggcggacctgccgtgtggaaaaccagactgagatgcgccctgaacaagagcagcgagttcaaagaggtgcccgagcggggcagaatggatgtggccgaaccttacaaggtgtaccagctgctgcctcctggcatcgtgtga

SEQ ID NO: 65 - RNA, 363 nt, Artificial Sequence
  Codon optimised RNA encoding IRF9
auggcuucuggcagagccagaugcacccggaagcugagaaacugggucgucgaacagguggaaagcggacaguucccuggcgugugcugggaugauaccgccaagacaauguucagaauccccuggaagcacgccggcaagcaggacuucagagaagaucaggacgccgccuucuucaaggccugggccaucuucaagggcaaguacaaagagggcgacacaggcggaccugccguguggaaaaccagacugagaugcgcccugaacaagagcagcgaguucaaagaggugcccgagcggggcagaauggauguggccgaaccuuacaagguguaccagcugcugccuccuggcaucguguga

SEQ ID NO: 66 - protein, 750 aa, Homo sapiens
MSQWYELQQLDSKFLEQVHQLYDDSFPMEIRQYLAQWLEKQDWEHAANDVSFATIRFHDLLSQLDDQYSRFSLENNFLLQHNIRKSKRNLQDNFQEDPIQMSMIIYSCLKEERKILENAQRFNQAQSGNIQSTVMLDKQKELDSKVRNVKDKVMCIEHEIKSLEDLQDEYDFKCKTLQNREHETNGVAKSDQKQEQLLLKKMYLMLDNKRKEVVHKIIELLNVTELTQNALINDELVEWKRRQQSACIGGPPNACLDQLQNWFTIVAESLQQVRQQLKKLEELEQKYTYEHDPITKNKQVLWDRTFSLFQQLIQSSFVVERQPCMPTHPQRPLVLKTGVQFTVKLRLLVKLQELNYNLKVKVLFDKDVNERNTVKGFRKFNILGTHTKVMNMEESTNGSLAAEFRHLQLKEQKNAGTRTNEGPLIVTEELHSLSFETQLCQPGLVIDLETTSLPVVVISNVSQLPSGWASILWYNMLVAEPRNLSFFLTPPCARWAQLSEVLSWQFSSVTKRGLNVDQLNMLGEKLLGPNASPDGLIPWTRFCKENINDKNFPFWLWIESILELIKKHLLPLWNDGCIMGFISKERERALLKDQQPGTFLLRFSESSREGAITFTWVERSQNGGEPDFHAVEPYTKKELSAVTFPDIIRNYKVMAAENIPENPLKYLYPNIDKDHAFGKYYSRPKEAPEPMELDGPKGTGFIKTELISVSEVHPSRLQTTDNLLPMSPEEFDEVSRIVGSVEFDSMMNTV

SEQ ID NO: 67 - DNA, 2250 bp, Homo sapiens
atgtctcagtggtacgaacttcagcagcttgactcaaaattcctggagcaggttcaccagctttatgatgacagttttcccatggaaatcagacagtacctggcacagtggttagaaaagcaagactgggagcacgctgccaatgatgtttcatttgccaccatccgttttcatgacctcctgtcacagctggatgatcaatatagtcgcttttctttggagaataacttcttgctacagcataacataaggaaaagcaagcgtaatcttcaggataattttcaggaagacccaatccagatgtctatgatcatttacagctgtctgaaggaagaaaggaaaattctggaaaacgcccagagatttaatcaggctcagtcggggaatattcagagcacagtgatgttagacaaacagaaagagcttgacagtaaagtcagaaatgtgaaggacaaggttatgtgtatagagcatgaaatcaagagcctggaagatttacaagatgaatatgacttcaaatgcaaaaccttgcagaacagagaacacgagaccaatggtgtggcaaagagtgatcagaaacaagaacagctgttactcaagaagatgtatttaatgcttgacaataagagaaaggaagtagttcacaaaataatagagttgctgaatgtcactgaacttacccagaatgccctgattaatgatgaactagtggagtggaagcggagacagcagagcgcctgtattggggggccgcccaatgcttgcttggatcagctgcagaactggttcactatagttgcggagagtctgcagcaagttcggcagcagcttaaaaagttggaggaattggaacagaaatacacctacgaacatgaccctatcacaaaaaacaaacaagtgttatgggaccgcaccttcagtcttttccagcagctcattcagagctcgtttgtggtggaaagacagccctgcatgccaacgcaccctcagaggccgctggtcttgaagacaggggtccagttcactgtgaagttgagactgttggtgaaattgcaagagctgaattataatttgaaagtcaaagtcttatttgataaagatgtgaatgagagaaatacagtaaaaggatttaggaagttcaacattttgggcacgcacacaaaagtgatgaacatggaggagtccaccaatggcagtctggcggctgaatttcggcacctgcaattgaaagaacagaaaaatgctggcaccagaacgaatgagggtcctctcatcgttactgaagagcttcactcccttagttttgaaacccaattgtgccagcctggtttggtaattgacctcgagacgacctctctgcccgttgtggtgatctccaacgtcagccagctcccgagcggttgggcctccatcctttggtacaacatgctggtggcggaacccaggaatctgtccttcttcctgactccaccatgtgcacgatgggctcagctttcagaagtgctgagttggcagttttcttctgtcaccaaaagaggtctcaatgtggaccagctgaacatgttgggagagaagcttcttggtcctaacgccagccccgatggtctcattccgtggacgaggttttgtaaggaaaatataaatgataaaaattttcccttctggctttggattgaaagcatcctagaactcattaaaaaacacctgctccctctctggaatgatgggtgcatcatgggcttcatcagcaaggagcgagagcgtgccctgttgaaggaccagcagccggggaccttcctgctgcggttcagtgagagctcccgggaaggggccatcacattcacatgggtggagcggtcccagaacggaggcgaacctgacttccatgcggttgaaccctacacgaagaaagaactttctgctgttactttccctgacatcattcgcaattacaaagtcatggctgctgagaatattcctgagaatcccctgaagtatctgtatccaaatattgacaaagaccatgcctttggaaagtattactccaggccaaaggaagcaccagagccaatggaacttgatggccctaaaggaactggatttatcaagactgagttgatttctgtgtctgaagttcacccttctagacttcagaccacagacaacctgctccccatgtctcctgaggagtttgacgaggtgtctcggatagtgggctctgtagaattcgacagtatgatgaacacagta

SEQ ID NO: 68 - RNA, 2250 nt, Homo sapiens
augucucagugguacgaacuucagcagcuugacucaaaauuccuggagcagguucaccagcuuuaugaugacaguuuucccauggaaaucagacaguaccuggcacagugguuagaaaagcaagacugggagcacgcugccaaugauguuucauuugccaccauccguuuucaugaccuccugucacagcuggaugaucaauauagucgcuuuucuuuggagaauaacuucuugcuacagcauaacauaaggaaaagcaagcguaaucuucaggauaauuuucaggaagacccaauccagaugucuaugaucauuuacagcugucugaaggaagaaaggaaaauucuggaaaacgcccagagauuuaaucaggcucagucggggaauauucagagcacagugauguuagacaaacagaaagagcuugacaguaaagucagaaaugugaaggacaagguuauguguauagagcaugaaaucaagagccuggaagauuuacaagaugaauaugacuucaaaugcaaaaccuugcagaacagagaacacgagaccaaugguguggcaaagagugaucagaaacaagaacagcuguuacucaagaagauguauuuaaugcuugacaauaagagaaaggaaguaguucacaaaauaauagaguugcugaaugucacugaacuuacccagaaugcccugauuaaugaugaacuaguggaguggaagcggagacagcagagcgccuguauuggggggccgcccaaugcuugcuuggaucagcugcagaacugguucacuauaguugcggagagucugcagcaaguucggcagcagcuuaaaaaguuggaggaauuggaacagaaauacaccuacgaacaugacccuaucacaaaaaacaaacaaguguuaugggaccgcaccuucagucuuuuccagcagcucauucagagcucguuugugguggaaagacagcccugcaugccaacgcacccucagaggccgcuggucuugaagacagggguccaguucacugugaaguugagacuguuggugaaauugcaagagcugaauuauaauuugaaagucaaagucuuauuugauaaagaugugaaugagagaaauacaguaaaaggauuuaggaaguucaacauuuugggcacgcacacaaaagugaugaacauggaggaguccaccaauggcagucuggcggcugaauuucggcaccugcaauugaaagaacagaaaaaugcuggcaccagaacgaaugaggguccucucaucguuacugaagagcuucacucccuuaguuuugaaacccaauugugccagccugguuugguaauugaccucgagacgaccucucugcccguuguggugaucuccaacgucagccagcucccgagcgguugggccuccauccuuugguacaacaugcugguggcggaacccaggaaucuguccuucuuccugacuccaccaugugcacgaugggcucagcuuucagaagugcugaguuggcaguuuucuucugucaccaaaagaggucucaauguggaccagcugaacauguugggagagaagcuucuugguccuaacgccagccccgauggucucauuccguggacgagguuuuguaaggaaaauauaaaugauaaaaauuuucccuucuggcuuuggauugaaagcauccuagaacucauuaaaaaacaccugcucccucucuggaaugaugggugcaucaugggcuucaucagcaaggagcgagagcgugcccuguugaaggaccagcagccggggaccuuccugcugcgguucagugagagcucccgggaaggggccaucacauucacauggguggagcggucccagaacggaggcgaaccugacuuccaugcgguugaacccuacacgaagaaagaacuuucugcuguuacuuucccugacaucauucgcaauuacaaagucauggcugcugagaauauuccugagaauccccugaaguaucuguauccaaauauugacaaagaccaugccuuuggaaaguauuacuccaggccaaaggaagcaccagagccaauggaacuugauggcccuaaaggaacuggauuuaucaagacugaguugauuucugugucugaaguucacccuucuagacuucagaccacagacaaccugcuccccaugucuccugaggaguuugacgaggugucucggauagugggcucuguagaauucgacaguaugaugaacacagua

SEQ ID NO: 69 - DNA, 2253 bp, Artificial Sequence
  Codon optimised DNA encoding STAT1
atgagccagtggtacgagctgcagcagctggacagcaagttcctggaacaggtgcaccagctgtacgacgacagcttccccatggaaatccggcagtatctggcccagtggctggaaaagcaggattgggaacacgccgccaacgacgtgtccttcgccaccatcagattccacgacctgctgagccagctggacgaccagtacagcagattcagcctggaaaacaacttcctgctccagcacaacatccggaagtccaagcggaacctgcaggacaacttccaagaggaccccatccagatgtccatgatcatctacagctgcctgaaagaggaacggaagatcctggaaaatgcccagcggttcaatcaggcccagagcggcaatatccagagcaccgtgatgctggacaagcagaaagaactggactccaaagtgcggaacgtcaaggacaaagtgatgtgcatcgagcacgagatcaagagcctggaagatctgcaggacgagtacgacttcaagtgcaagaccctgcagaaccgggaacacgagacaaacggcgtggccaagagcgaccagaagcaagaacagctgctcctgaagaaaatgtacctgatgctcgacaacaaacggaaagaggtggtccacaagatcatcgagctgctgaacgtgaccgagctgacccagaacgccctgatcaacgacgagctggtggaatggaagcggagacagcagtctgcctgtatcggcggacctcctaatgcctgcctggaccagctgcagaactggttcacaatcgtggccgagagcctgcagcaagtgcgccagcagctgaagaagctggaagaactcgagcagaagtacacctacgagcacgaccccatcaccaagaacaaacaggtgctgtgggacagaaccttcagcctgttccaacagctgatccagtccagcttcgtggtggaaagacagccctgcatgcctacacaccctcagaggccactggtgctgaaaaccggcgtgcagttcaccgtgaagctgcggctgctggtcaagctgcaagagctgaactacaacctgaaagtgaaggtgctgttcgacaaggacgtgaacgagcggaacaccgtgaaaggcttccgcaagttcaacatcctgggcacccacacaaaagtgatgaacatggaagagagcaccaacggcagcctggccgccgagtttagacacctccagctgaaagagcagaagaacgccggcaccaggaccaatgagggacctctgatcgtgacagaggaactgcacagcctgagcttcgaaacccagctgtgtcagccaggcctcgtgatcgatctggaaaccacaagcctgcctgtggtggtcatcagcaatgtgtcccagctgccttctggctgggccagcatcctgtggtacaacatgctggtggccgagcctcggaacctgtccttctttctgacccctccatgtgccagatgggcccagctgtctgaagtgctgagctggcagtttagcagcgtgaccaagaggggcctgaatgtcgaccagctgaatatgctgggcgagaagctgctgggccccaacgcttctcctgatggactgatcccttggaccagattctgcaaagagaatatcaacgacaagaacttcccgttctggctgtggatcgagagcatcctggaactgatcaagaaacatctgctgcccctgtggaacgacggctgcatcatgggcttcatctccaaagagagagagcgggccctgctgaaggatcagcagccaggcacattcctgctgcggtttagcgagtctagcagagagggcgccatcacctttacctgggtcgagagatctcagaacggcggcgagcctgattttcacgccgtggaaccctacaccaaaaaagaactgagcgccgtgacattccccgacatcatccggaactacaaagtcatggccgctgagaatatccccgagaatcccctgaagtatctgtaccccaacatcgataaggaccacgccttcggcaagtactacagcagacccaaagaggcccctgagcctatggaactggatggccctaaaggcaccggcttcatcaagacagagctgatctccgtgtccgaggtgcaccctagcagactgcagaccaccgataacctgctgcctatgagccccgaggaattcgacgaggtgtccagaatcgtgggcagcgtggaattcgatagcatgatgaataccgtgtga

SEQ ID NO: 70 - RNA, 2253 nt, Artificial Sequence
  Codon optimised RNA encoding STAT1
augagccagugguacgagcugcagcagcuggacagcaaguuccuggaacaggugcaccagcuguacgacgacagcuuccccauggaaauccggcaguaucuggcccaguggcuggaaaagcaggauugggaacacgccgccaacgacguguccuucgccaccaucagauuccacgaccugcugagccagcuggacgaccaguacagcagauucagccuggaaaacaacuuccugcuccagcacaacauccggaaguccaagcggaaccugcaggacaacuuccaagaggaccccauccagauguccaugaucaucuacagcugccugaaagaggaacggaagauccuggaaaaugcccagcgguucaaucaggcccagagcggcaauauccagagcaccgugaugcuggacaagcagaaagaacuggacuccaaagugcggaacgucaaggacaaagugaugugcaucgagcacgagaucaagagccuggaagaucugcaggacgaguacgacuucaagugcaagacccugcagaaccgggaacacgagacaaacggcguggccaagagcgaccagaagcaagaacagcugcuccugaagaaaauguaccugaugcucgacaacaaacggaaagaggugguccacaagaucaucgagcugcugaacgugaccgagcugacccagaacgcccugaucaacgacgagcugguggaauggaagcggagacagcagucugccuguaucggcggaccuccuaaugccugccuggaccagcugcagaacugguucacaaucguggccgagagccugcagcaagugcgccagcagcugaagaagcuggaagaacucgagcagaaguacaccuacgagcacgaccccaucaccaagaacaaacaggugcugugggacagaaccuucagccuguuccaacagcugauccaguccagcuucgugguggaaagacagcccugcaugccuacacacccucagaggccacuggugcugaaaaccggcgugcaguucaccgugaagcugcggcugcuggucaagcugcaagagcugaacuacaaccugaaagugaaggugcuguucgacaaggacgugaacgagcggaacaccgugaaaggcuuccgcaaguucaacauccugggcacccacacaaaagugaugaacauggaagagagcaccaacggcagccuggccgccgaguuuagacaccuccagcugaaagagcagaagaacgccggcaccaggaccaaugagggaccucugaucgugacagaggaacugcacagccugagcuucgaaacccagcugugucagccaggccucgugaucgaucuggaaaccacaagccugccugugguggucaucagcaaugugucccagcugccuucuggcugggccagcauccugugguacaacaugcugguggccgagccucggaaccuguccuucuuucugaccccuccaugugccagaugggcccagcugucugaagugcugagcuggcaguuuagcagcgugaccaagaggggccugaaugucgaccagcugaauaugcugggcgagaagcugcugggccccaacgcuucuccugauggacugaucccuuggaccagauucugcaaagagaauaucaacgacaagaacuucccguucuggcuguggaucgagagcauccuggaacugaucaagaaacaucugcugccccuguggaacgacggcugcaucaugggcuucaucuccaaagagagagagcgggcccugcugaaggaucagcagccaggcacauuccugcugcgguuuagcgagucuagcagagagggcgccaucaccuuuaccugggucgagagaucucagaacggcggcgagccugauuuucacgccguggaacccuacaccaaaaaagaacugagcgccgugacauuccccgacaucauccggaacuacaaagucauggccgcugagaauauccccgagaauccccugaaguaucuguaccccaacaucgauaaggaccacgccuucggcaaguacuacagcagacccaaagaggccccugagccuauggaacuggauggcccuaaaggcaccggcuucaucaagacagagcugaucuccguguccgaggugcacccuagcagacugcagaccaccgauaaccugcugccuaugagccccgaggaauucgacgagguguccagaaucgugggcagcguggaauucgauagcaugaugaauaccguguga

SEQ ID NO: 71 - protein, 183 aa, Homo sapiens
VLETPVESQQHEIESRILDLRAMMEKLVKSISQLKDQQDVFCFRYKIQAKGKTPSLDPHQTKEQKILQETLNELDKRRKEVLDASKALLGRLTTLIELLLPKLEEWKAQQQKACIRAPIDHGLEQLETWFTAGAKLLFHLRQLLKELKGLSCLVSYQDDPLTKGVDLRNAQVTELLQRLLHRA

SEQ ID NO: 72 - DNA, 549 bp, Homo sapiens
gttctcgaaacacctgtggagagccagcaacatgagattgaatcccggatcctggatttaagggctatgatggagaagctggtaaaatccatcagccaactgaaagaccagcaggatgtcttctgcttccgatataagatccaggccaaagggaagacaccctctctggacccccatcagaccaaagagcagaagattctgcaggaaactctcaatgaactggacaaaaggagaaaggaggtgctggatgcctccaaagcactgctaggccgattaactaccctaatcgagctactgctgccaaagttggaggagtggaaggcccagcagcaaaaagcctgcatcagagctcccattgaccacgggttggaacagctggagacatggttcacagctggagcaaagctgttgtttcacctgaggcagctgctgaaggagctgaagggactgagttgcctggttagctatcaggatgaccctctgaccaaaggggtggacctacgcaacgcccaggtcacagagttgctacagcgtctgctccacagagcc

SEQ ID NO: 73 - RNA, 549 nt, Homo sapiens
guucucgaaacaccuguggagagccagcaacaugagauugaaucccggauccuggauuuaagggcuaugauggagaagcugguaaaauccaucagccaacugaaagaccagcaggaugucuucugcuuccgauauaagauccaggccaaagggaagacacccucucuggacccccaucagaccaaagagcagaagauucugcaggaaacucucaaugaacuggacaaaaggagaaaggaggugcuggaugccuccaaagcacugcuaggccgauuaacuacccuaaucgagcuacugcugccaaaguuggaggaguggaaggcccagcagcaaaaagccugcaucagagcucccauugaccacggguuggaacagcuggagacaugguucacagcuggagcaaagcuguuguuucaccugaggcagcugcugaaggagcugaagggacugaguugccugguuagcuaucaggaugacccucugaccaaagggguggaccuacgcaacgcccaggucacagaguugcuacagcgucugcuccacagagcc

SEQ ID NO: 74 - DNA, 555 bp, Artificial Sequence
  Codon optimised DNA encoding STAT2
atggtgctggaaacccctgtggaaagccagcagcacgagatcgagagcagaatcctggacctgcgggccatgatggaaaagctggtcaagagcatcagccagctgaaggaccagcaggacgtgttctgcttccggtacaagatccaggccaagggcaagacccctagcctggatcctcaccagaccaaagagcagaagatcctgcaagagacactgaacgagctggacaagcggcggaaagaagtgctggacgcctctaaagctctgctgggcagactgaccactctgatcgaactgctgctgcccaagctggaagagtggaaggcccagcaacagaaggcctgcatcagagcccctatcgaccacggactggaacagctggaaacatggtttaccgctggcgccaagctgctgttccacctgagacagctgctgaaagagctgaagggcctgagctgcctggtgtcctaccaggatgaccctctgaccaaaggcgtggacctgagaaacgcccaagtgaccgaactgctccagcggctgctgcatagagcttga

SEQ ID NO: 75 - RNA, 555 nt, Artificial Sequence
  Codon optimised RNA encoding STAT2
auggugcuggaaaccccuguggaaagccagcagcacgagaucgagagcagaauccuggaccugcgggccaugauggaaaagcuggucaagagcaucagccagcugaaggaccagcaggacguguucugcuuccgguacaagauccaggccaagggcaagaccccuagccuggauccucaccagaccaaagagcagaagauccugcaagagacacugaacgagcuggacaagcggcggaaagaagugcuggacgccucuaaagcucugcugggcagacugaccacucugaucgaacugcugcugcccaagcuggaagaguggaaggcccagcaacagaaggccugcaucagagccccuaucgaccacggacuggaacagcuggaaacaugguuuaccgcuggcgccaagcugcuguuccaccugagacagcugcugaaagagcugaagggccugagcugccugguguccuaccaggaugacccucugaccaaaggcguggaccugagaaacgcccaagugaccgaacugcuccagcggcugcugcauagagcuuga

SEQ ID NO: 76 - protein, 851 aa, Homo sapiens
MAQWEMLQNLDSPFQDQLHQLYSHSLLPVDIRQYLAVWIEDQNWQEAALGSDDSKATMLFFHFLDQLNYECGRCSQDPESLLLQHNLRKFCRDIQPFSQDPTQLAEMIFNLLLEEKRILIQAQRAQLEQGEPVLETPVESQQHEIESRILDLRAMMEKLVKSISQLKDQQDVFCDRYKIQAKGKTPSLDPHQTKEQKILQETLNELDKRRKEVLDASKALLGRLTTLIELLLPKLEEWKAQQQKACIRAPIDHGLEQLETWFTAGAKLLFHLRQLLKELKGLSCLVSYQDDPLTKGVDLRNAQVTELLQRLLHRAFVVETQPCMPQTPHRPLILKTGSKFTVRTRLLVRLQEGNESLTVEVSIDRNPPQLQGFRKFNILTSNQKTLTPEKGQSQGLIWDFGYLTLVEQRSGGSGKGSNKGPLGVTEELHIISFTVKYTYQGLKQELKTDTLPVVIISNMNQLSIAWASVLWFNLLSPNLQNQQFFSNPPKAPWSLLGPALSWQFSSYVGRGLNSDQLSMLRNKLFGQNCRTEDPLLSWADFTKRESPPGKLPFWTWLDKILELVHDHLKDLWNDGRIMGFVSRSQERRLLKKTMSGTFLLRFSESSEGGITCSWVEHQDDDKVLIYSVQPYTKEVLQSLPLTEIIRHYQLLTEENIPENPLRFLYPRIPRDEAFGCYYQEKVNLQERRKYLKHRLIVVSNRQVDELQQPLELKPEPELESLELELGLVPEPELSLDLEPLLKAGLDLGPELESVLESTLEPVIEPTLCMVSQTVPEPDQGPVSQPVPEPDLPCDLRHLNTEPMEIFRNCVKIEEIMPNGDPLLAGQNTVDEVYVSRPSHFYTDGPLMPSDF

SEQ ID NO: 77 - DNA, 2553 bp, Homo sapiens
atggcgcagtgggaaatgctgcagaatcttgacagcccctttcaggatcagctgcaccagctttactcgcacagcctcctgcctgtggacattcgacagtacttggctgtctggattgaagaccagaactggcaggaagctgcacttgggagtgatgattccaaggctaccatgctattcttccacttcttggatcagctgaactatgagtgtggccgttgcagccaggacccagagtccttgttgctgcagcacaatttgcggaaattctgccgggacattcagcccttttcccaggatcctacccagttggctgagatgatctttaacctccttctggaagaaaaaagaattttgatccaggctcagagggcccaattggaacaaggagagccagttctcgaaacacctgtggagagccagcaacatgagattgaatcccggatcctggatttaagggctatgatggagaagctggtaaaatccatcagccaactgaaagaccagcaggatgtcttctgcgaccgatataagatccaggccaaagggaagacaccctctctggacccccatcagaccaaagagcagaagattctgcaggaaactctcaatgaactggacaaaaggagaaaggaggtgctggatgcctccaaagcactgctaggccgattaactaccctaatcgagctactgctgccaaagttggaggagtggaaggcccagcagcaaaaagcctgcatcagagctcccattgaccacgggttggaacagctggagacatggttcacagctggagcaaagctgttgtttcacctgaggcagctgctgaaggagctgaagggactgagttgcctggttagctatcaggatgaccctctgaccaaaggggtggacctacgcaacgcccaggtcacagagttgctacagcgtctgctccacagagcctttgtggtagaaacccagccctgcatgccccaaactccccatcgacccctcatcctcaagactggcagcaagttcaccgtccgaacaaggctgctggtgagactccaggaaggcaatgagtcactgactgtggaagtctccattgacaggaatcctcctcaattacaaggcttccggaagttcaacattctgacttcaaaccagaaaactttgacccccgagaaggggcagagtcagggtttgatttgggactttggttacctgactctggtggagcaacgttcaggtggttcaggaaagggcagcaataaggggccactaggtgtgacagaggaactgcacatcatcagcttcacggtcaaatatacctaccagggtctgaagcaggagctgaaaacggacaccctccctgtggtgattatttccaacatgaaccagctctcaattgcctgggcttcagttctctggttcaatttgctcagcccaaaccttcagaaccagcagttcttctccaacccccccaaggccccctggagcttgctgggccctgctctcagttggcagttctcctcctatgttggccgaggcctcaactcagaccagctgagcatgctgagaaacaagctgttcgggcagaactgtaggactgaggatccattattgtcctgggctgacttcactaagcgagagagccctcctggcaagttaccattctggacatggctggacaaaattctggagttggtacatgaccacctgaaggatctctggaatgatggacgcatcatgggctttgtgagtcggagccaggagcgccggctgctgaagaagaccatgtctggcacctttctactgcgcttcagtgaatcgtcagaagggggcattacctgctcctgggtggagcaccaggatgatgacaaggtgctcatctactctgtgcaaccgtacacgaaggaggtgctgcagtcactcccgctgactgaaatcatccgccattaccagttgctcactgaggagaatatacctgaaaacccactgcgcttcctctatccccgaatcccccgggatgaagcttttgggtgctactaccaggagaaagttaatctccaggaacggaggaaatacctgaaacacaggctcattgtggtctctaatagacaggtggatgaactgcaacaaccgctggagcttaagccagagccagagctggagtcattagagctggaactagggctggtgccagagccagagctcagcctggacttagagccactgctgaaggcagggctggatctggggccagagctagagtctgtgctggagtccactctggagcctgtgatagagcccacactatgcatggtatcacaaacagtgccagagccagaccaaggacctgtatcacagccagtgccagagccagatttgccctgtgatctgagacatttgaacactgagccaatggaaatcttcagaaactgtgtaaagattgaagaaatcatgccgaatggtgacccactgttggctggccagaacaccgtggatgaggtttacgtctcccgccccagccacttctacactgatggacccttgatgccttctgacttc

SEQ ID NO: 78 - RNA, 2553 nt, Homo sapiens
auggcgcagugggaaaugcugcagaaucuugacagccccuuucaggaucagcugcaccagcuuuacucgcacagccuccugccuguggacauucgacaguacuuggcugucuggauugaagaccagaacuggcaggaagcugcacuugggagugaugauuccaaggcuaccaugcuauucuuccacuucuuggaucagcugaacuaugaguguggccguugcagccaggacccagaguccuuguugcugcagcacaauuugcggaaauucugccgggacauucagcccuuuucccaggauccuacccaguuggcugagaugaucuuuaaccuccuucuggaagaaaaaagaauuuugauccaggcucagagggcccaauuggaacaaggagagccaguucucgaaacaccuguggagagccagcaacaugagauugaaucccggauccuggauuuaagggcuaugauggagaagcugguaaaauccaucagccaacugaaagaccagcaggaugucuucugcgaccgauauaagauccaggccaaagggaagacacccucucuggacccccaucagaccaaagagcagaagauucugcaggaaacucucaaugaacuggacaaaaggagaaaggaggugcuggaugccuccaaagcacugcuaggccgauuaacuacccuaaucgagcuacugcugccaaaguuggaggaguggaaggcccagcagcaaaaagccugcaucagagcucccauugaccacggguuggaacagcuggagacaugguucacagcuggagcaaagcuguuguuucaccugaggcagcugcugaaggagcugaagggacugaguugccugguuagcuaucaggaugacccucugaccaaagggguggaccuacgcaacgcccaggucacagaguugcuacagcgucugcuccacagagccuuugugguagaaacccagcccugcaugccccaaacuccccaucgaccccucauccucaagacuggcagcaaguucaccguccgaacaaggcugcuggugagacuccaggaaggcaaugagucacugacuguggaagucuccauugacaggaauccuccucaauuacaaggcuuccggaaguucaacauucugacuucaaaccagaaaacuuugacccccgagaaggggcagagucaggguuugauuugggacuuugguuaccugacucugguggagcaacguucaggugguucaggaaagggcagcaauaaggggccacuaggugugacagaggaacugcacaucaucagcuucacggucaaauauaccuaccagggucugaagcaggagcugaaaacggacacccucccuguggugauuauuuccaacaugaaccagcucucaauugccugggcuucaguucucugguucaauuugcucagcccaaaccuucagaaccagcaguucuucuccaacccccccaaggcccccuggagcuugcugggcccugcucucaguuggcaguucuccuccuauguuggccgaggccucaacucagaccagcugagcaugcugagaaacaagcuguucgggcagaacuguaggacugaggauccauuauuguccugggcugacuucacuaagcgagagagcccuccuggcaaguuaccauucuggacauggcuggacaaaauucuggaguugguacaugaccaccugaaggaucucuggaaugauggacgcaucaugggcuuugugagucggagccaggagcgccggcugcugaagaagaccaugucuggcaccuuucuacugcgcuucagugaaucgucagaagggggcauuaccugcuccuggguggagcaccaggaugaugacaaggugcucaucuacucugugcaaccguacacgaaggaggugcugcagucacucccgcugacugaaaucauccgccauuaccaguugcucacugaggagaauauaccugaaaacccacugcgcuuccucuauccccgaaucccccgggaugaagcuuuugggugcuacuaccaggagaaaguuaaucuccaggaacggaggaaauaccugaaacacaggcucauuguggucucuaauagacagguggaugaacugcaacaaccgcuggagcuuaagccagagccagagcuggagucauuagagcuggaacuagggcuggugccagagccagagcucagccuggacuuagagccacugcugaaggcagggcuggaucuggggccagagcuagagucugugcuggaguccacucuggagccugugauagagcccacacuaugcaugguaucacaaacagugccagagccagaccaaggaccuguaucacagccagugccagagccagauuugcccugugaucugagacauuugaacacugagccaauggaaaucuucagaaacuguguaaagauugaagaaaucaugccgaauggugacccacuguuggcuggccagaacaccguggaugagguuuacgucucccgccccagccacuucuacacugauggacccuugaugccuucugacuuc

SEQ ID NO: 79 - DNA, 2556 bp, Artificial Sequence
  Codon optimised DNA encoding STAT2
atggcccagtgggagatgctgcagaacctggacagccccttccaggatcagctgcaccagctgtactcccactctctgctgcccgtggacatcagacagtatctggccgtgtggatcgaggaccagaactggcaagaagccgctctgggcagcgacgatagcaaggccacaatgctgttcttccacttcctggaccagctgaactacgagtgcggcagatgcagccaggatccagaaagtctgctgctccagcacaacctgcggaagttctgcagagacatccagccattctctcaggaccccacacagctggccgagatgatcttcaacctgctgctggaagagaagcggatcctgattcaggcccagagagcccagctggaacagggcgaacctgtcctggaaacccctgtggaatctcagcagcacgagatcgagagccggatcctggatctgcgggccatgatggaaaagctggtcaagagcatcagccagctgaaggaccagcaggacgtgttctgcgaccggtacaagatccaggccaagggcaagacccctagcctggatcctcaccagaccaaagagcagaagatcctgcaagagacactgaacgagctggacaagcggcggaaagaagtgctggacgcctctaaagctctgctgggcagactgaccactctgatcgaactgctgctgcccaagctggaagaatggaaggcacagcagcagaaggcctgcatcagagcccctatcgatcacggcctggaacagctggaaacctggtttacagccggcgctaagctgctgttccacctgagacagctgctgaaagagctgaagggcctgagctgcctggtgtcctaccaggatgaccctctgaccaaaggcgtggacctgagaaacgcccaagtgaccgaactgctccagagactgctgcacagagccttcgtggtggaaacccagccttgcatgccccagacacctcacagacccctgatcctgaaaaccggcagcaagttcaccgtgcggaccagactgctcgtgcgactgcaagagggcaatgagagcctgaccgtggaagtgtccatcgacagaaaccctccacagctgcagggcttcagaaagttcaacatcctgaccagcaaccagaaaaccctgacacctgagaagggccagagccagggactgatctgggacttcggctacctgacactggtcgagcagagatctggcggctctggcaagggctctaacaagggacctctgggcgtgaccgaggaactgcacatcatcagcttcaccgtgaagtacacctaccagggcctgaagcaagaactcaagaccgacacactgcccgtcgtgatcatcagcaacatgaaccagctgtctatcgcctgggccagcgtgctgtggttcaatctgctgagccccaacctgcagaatcagcagttcttcagcaaccctcctaaggctccttggagcctgctgggacctgctctgagctggcagtttagcagctatgtcggcagaggcctgaacagcgatcagctgagcatgctgcggaacaagctgttcggccagaactgcaggaccgaggatccactgctgagctgggccgacttcaccaagagagagagccctccaggcaagctgcccttctggacttggctggacaaaatcctggaactggtgcacgaccacctgaaggatctgtggaacgacggccggatcatgggcttcgtgtccagatctcaagagcgcagactgctgaaaaagacaatgagcggcaccttcctgctgcggttcagcgaatctagcgaaggcggcatcacctgtagctgggtcgaacaccaggacgacgacaaggtgctgatctacagcgtgcagccctacaccaaagaggtgctgcaaagcctgcctctgaccgagatcatccggcactaccagctgctcaccgaggaaaacatccccgagaatcctctgcggtttctgtaccctcggatccccagagatgaggcctttggctgctactaccaagagaaagtgaatctgcaagagcggcgcaagtacctgaagcacagactgatcgtggtgtccaacagacaggtggacgagctgcagcagccactggaactgaagcctgagccagagctggaaagcctcgagctggaacttggactggtgcccgagcctgaactgtctctggatctggaacctctgctgaaggccggactggacctcggacctgaactggaaagcgtgctggaatccacactggaacctgtgatcgagcccacactgtgcatggtgtctcagaccgtgcctgaaccagatcagggcccagtgtctcagcctgttcctgagcctgatctgccctgcgatctgaggcacctgaacaccgagcctatggaaatcttccggaactgcgtgaagatcgaggaaatcatgcccaacggcgaccctctgctggccggacagaataccgtggatgaagtgtacgtgtcccggcctagccacttctacacagacggacctctgatgcccagcgacttctga

SEQ ID NO: 80 - RNA, 2556 nt, Artificial Sequence
  Codon optimised RNA encoding STAT2
auggcccagugggagaugcugcagaaccuggacagccccuuccaggaucagcugcaccagcuguacucccacucucugcugcccguggacaucagacaguaucuggccguguggaucgaggaccagaacuggcaagaagccgcucugggcagcgacgauagcaaggccacaaugcuguucuuccacuuccuggaccagcugaacuacgagugcggcagaugcagccaggauccagaaagucugcugcuccagcacaaccugcggaaguucugcagagacauccagccauucucucaggaccccacacagcuggccgagaugaucuucaaccugcugcuggaagagaagcggauccugauucaggcccagagagcccagcuggaacagggcgaaccuguccuggaaaccccuguggaaucucagcagcacgagaucgagagccggauccuggaucugcgggccaugauggaaaagcuggucaagagcaucagccagcugaaggaccagcaggacguguucugcgaccgguacaagauccaggccaagggcaagaccccuagccuggauccucaccagaccaaagagcagaagauccugcaagagacacugaacgagcuggacaagcggcggaaagaagugcuggacgccucuaaagcucugcugggcagacugaccacucugaucgaacugcugcugcccaagcuggaagaauggaaggcacagcagcagaaggccugcaucagagccccuaucgaucacggccuggaacagcuggaaaccugguuuacagccggcgcuaagcugcuguuccaccugagacagcugcugaaagagcugaagggccugagcugccugguguccuaccaggaugacccucugaccaaaggcguggaccugagaaacgcccaagugaccgaacugcuccagagacugcugcacagagccuucgugguggaaacccagccuugcaugccccagacaccucacagaccccugauccugaaaaccggcagcaaguucaccgugcggaccagacugcucgugcgacugcaagagggcaaugagagccugaccguggaaguguccaucgacagaaacccuccacagcugcagggcuucagaaaguucaacauccugaccagcaaccagaaaacccugacaccugagaagggccagagccagggacugaucugggacuucggcuaccugacacuggucgagcagagaucuggcggcucuggcaagggcucuaacaagggaccucugggcgugaccgaggaacugcacaucaucagcuucaccgugaaguacaccuaccagggccugaagcaagaacucaagaccgacacacugcccgucgugaucaucagcaacaugaaccagcugucuaucgccugggccagcgugcugugguucaaucugcugagccccaaccugcagaaucagcaguucuucagcaacccuccuaaggcuccuuggagccugcugggaccugcucugagcuggcaguuuagcagcuaugucggcagaggccugaacagcgaucagcugagcaugcugcggaacaagcuguucggccagaacugcaggaccgaggauccacugcugagcugggccgacuucaccaagagagagagcccuccaggcaagcugcccuucuggacuuggcuggacaaaauccuggaacuggugcacgaccaccugaaggaucuguggaacgacggccggaucaugggcuucguguccagaucucaagagcgcagacugcugaaaaagacaaugagcggcaccuuccugcugcgguucagcgaaucuagcgaaggcggcaucaccuguagcugggucgaacaccaggacgacgacaaggugcugaucuacagcgugcagcccuacaccaaagaggugcugcaaagccugccucugaccgagaucauccggcacuaccagcugcucaccgaggaaaacauccccgagaauccucugcgguuucuguacccucggauccccagagaugaggccuuuggcugcuacuaccaagagaaagugaaucugcaagagcggcgcaaguaccugaagcacagacugaucgugguguccaacagacagguggacgagcugcagcagccacuggaacugaagccugagccagagcuggaaagccucgagcuggaacuuggacuggugcccgagccugaacugucucuggaucuggaaccucugcugaaggccggacuggaccucggaccugaacuggaaagcgugcuggaauccacacuggaaccugugaucgagcccacacugugcauggugucucagaccgugccugaaccagaucagggcccagugucucagccuguuccugagccugaucugcccugcgaucugaggcaccugaacaccgagccuauggaaaucuuccggaacugcgugaagaucgaggaaaucaugcccaacggcgacccucugcuggccggacagaauaccguggaugaaguguacgugucccggccuagccacuucuacacagacggaccucugaugcccagcgacuucuga

SEQ ID NO: 81 - protein, 232 aa, Homo sapiens
MVDYSVWDHIEVSDDEDETHPNIDTASLFRWRHQARVERMEQFQKEKEELDRGCRECKRKVAECQRKLKELEVAEGGKAELERLQAEAQQLRKEERSWEQKLEEMRKKEKSMPWNVDTLSKDGFSKSMVNTKPEKTEEDSEEVREQKHKTFVEKYEKQIKHFGMLRRWDDSQKYLSDNVHLVCEETANYLVIWCIDLEVEEKCALMEQVAHQTIVMQFILELAKSLKVDPRA

SEQ ID NO: 82 - DNA, 696 bp, Homo sapiens
atggtggactacagcgtgtgggaccacattgaggtgtctgatgatgaagacgagacgcaccccaacatcgacacggccagtctcttccgctggcggcatcaggcccgggtggaacgcatggagcagttccagaaggagaaggaggaactggacaggggctgccgcgagtgcaagcgcaaggtggccgagtgccagaggaaactgaaggagctggaggtggccgagggcggcaaggcagagctggagcgcctgcaggccgaggcacagcagctgcgcaaggaggagcggagctgggagcagaagctggaggagatgcgcaagaaggagaagagcatgccctggaacgtggacacgctcagcaaagacggcttcagcaagagcatggtaaataccaagcccgagaagacggaggaggactcagaggaggtgagggagcagaaacacaagaccttcgtggaaaaatacgagaaacagatcaagcactttggcatgcttcgccgctgggatgacagccaaaagtacctgtcagacaacgtccacctggtgtgcgaggagacagccaattacctggtcatttggtgcattgacctagaggtggaggagaaatgtgcactcatggagcaggtggcccaccagacaatcgtcatgcaatttatcctggagctggccaagagcctaaaggtggacccccgggcc

SEQ ID NO: 83 - RNA, 696 nt, Homo sapiens
augguggacuacagcgugugggaccacauugaggugucugaugaugaagacgagacgcaccccaacaucgacacggccagucucuuccgcuggcggcaucaggcccggguggaacgcauggagcaguuccagaaggagaaggaggaacuggacaggggcugccgcgagugcaagcgcaagguggccgagugccagaggaaacugaaggagcuggagguggccgagggcggcaaggcagagcuggagcgccugcaggccgaggcacagcagcugcgcaaggaggagcggagcugggagcagaagcuggaggagaugcgcaagaaggagaagagcaugcccuggaacguggacacgcucagcaaagacggcuucagcaagagcaugguaaauaccaagcccgagaagacggaggaggacucagaggaggugagggagcagaaacacaagaccuucguggaaaaauacgagaaacagaucaagcacuuuggcaugcuucgccgcugggaugacagccaaaaguaccugucagacaacguccaccuggugugcgaggagacagccaauuaccuggucauuuggugcauugaccuagagguggaggagaaaugugcacucauggagcagguggcccaccagacaaucgucaugcaauuuauccuggagcuggccaagagccuaaagguggacccccgggcc

SEQ ID NO: 84 - DNA, 699 bp, Artificial Sequence
  Codon optimised DNA encoding HSP90
atggtggactacagcgtgtgggaccacatcgaggtgtccgacgacgaggatgagacacaccccaacatcgataccgccagcctgttcagatggcggcaccaggctagagtggaacggatggaacagttccagaaagagaaagaggaactggaccggggctgccgcgagtgcaaaagaaaagtggccgagtgccagcggaagctgaaagaactggaagtggctgaaggcggcaaggccgagctggaaagactgcaggctgaagcccagcagctgcgcaaagaggaaagaagctgggagcagaaactggaagagatgcgcaagaaagaaaaatccatgccgtggaacgtggacaccctgagcaaggacggcttcagcaagagcatggtcaacaccaagcctgagaaaaccgaagaggacagcgaggaagtgcgggaacagaaacacaagaccttcgtcgagaagtacgagaagcagatcaagcacttcggcatgctgcggagatgggacgacagccagaagtacctgagcgacaacgtgcacctcgtgtgcgaggaaaccgccaactacctggtcatctggtgcatcgatctcgaggtggaagagaagtgcgccctcatggaacaggtggcccaccagacaatcgtgatgcagttcatcctggaactggccaagagcctgaaggtggaccctagagcttga

SEQ ID NO: 85 - RNA, 699 nt, Artificial Sequence
  Codon optimised RNA encoding HSP90
augguggacuacagcgugugggaccacaucgagguguccgacgacgaggaugagacacaccccaacaucgauaccgccagccuguucagauggcggcaccaggcuagaguggaacggauggaacaguuccagaaagagaaagaggaacuggaccggggcugccgcgagugcaaaagaaaaguggccgagugccagcggaagcugaaagaacuggaaguggcugaaggcggcaaggccgagcuggaaagacugcaggcugaagcccagcagcugcgcaaagaggaaagaagcugggagcagaaacuggaagagaugcgcaagaaagaaaaauccaugccguggaacguggacacccugagcaaggacggcuucagcaagagcauggucaacaccaagccugagaaaaccgaagaggacagcgaggaagugcgggaacagaaacacaagaccuucgucgagaaguacgagaagcagaucaagcacuucggcaugcugcggagaugggacgacagccagaaguaccugagcgacaacgugcaccucgugugcgaggaaaccgccaacuaccuggucaucuggugcaucgaucucgagguggaagagaagugcgcccucauggaacagguggcccaccagacaaucgugaugcaguucauccuggaacuggccaagagccugaagguggacccuagagcuuga

SEQ ID NO: 86 - protein, 231 aa, Homo sapiens
MTWVSLLNQVGDRVSRNNFLGFPASELQARIRTYNQHYNNLLRGAVSQRLYILLPLDCGVPDNLSMADPNIRFLDKLPQQTGDHAGIKDRVYSNSIYELLENGQRAGTCVLEYATPLQTLFAMSQYSQAGFSREDRLEQAKLFCRTLEDILADAPESQNNCRLIAYQEPADDSSFSLSQEVLRHLRQEEKEEVTVGSLKTSAVPSTSTMSQEPELLISGMEKPLPLRTDFS

SEQ ID NO: 87 - DNA, 693 bp, Homo sapiens
atgacctgggtctcactcctgaatcaggtgggagatagggttagcaggaataacttcttgggcttccctgcctcagagctccaggcccggattcgaacttacaatcagcattacaacaacctgctacggggtgcagtgagccagcggctgtatattctcctcccattggactgtggggtgcctgataacctgagtatggctgaccccaacattcgcttcctggataaactgccccagcagaccggtgaccatgctggcatcaaggatcgggtttacagcaacagcatctatgagcttctggagaacgggcagcgggcgggcacctgtgtcctggagtacgccacccccttgcagactttgtttgccatgtcacaatacagtcaagctggctttagccgggaggataggcttgagcaggccaaactcttctgccggacacttgaggacatcctggcagatgcccctgagtctcagaacaactgccgcctcattgcctaccaggaacctgcagatgacagcagcttctcgctgtcccaggaggttctccggcacctgcggcaggaggaaaaggaagaggttactgtgggcagcttgaagacctcagcggtgcccagtacctccacgatgtcccaagagcctgagctcctcatcagtggaatggaaaagcccctccctctccgcacggatttctct

SEQ ID NO: 88 - RNA, 693 nt, Homo sapiens
augaccugggucucacuccugaaucaggugggagauaggguuagcaggaauaacuucuugggcuucccugccucagagcuccaggcccggauucgaacuuacaaucagcauuacaacaaccugcuacggggugcagugagccagcggcuguauauucuccucccauuggacuguggggugccugauaaccugaguauggcugaccccaacauucgcuuccuggauaaacugccccagcagaccggugaccaugcuggcaucaaggaucggguuuacagcaacagcaucuaugagcuucuggagaacgggcagcgggcgggcaccuguguccuggaguacgccacccccuugcagacuuuguuugccaugucacaauacagucaagcuggcuuuagccgggaggauaggcuugagcaggccaaacucuucugccggacacuugaggacauccuggcagaugccccugagucucagaacaacugccgccucauugccuaccaggaaccugcagaugacagcagcuucucgcugucccaggagguucuccggcaccugcggcaggaggaaaaggaagagguuacugugggcagcuugaagaccucagcggugcccaguaccuccacgaugucccaagagccugagcuccucaucaguggaauggaaaagccccucccucuccgcacggauuucucu

SEQ ID NO: 89 - DNA, 696 bp, Artificial Sequence
  Codon optimised DNA encoding STING-beta
atgacatgggtgtccctgctgaatcaagtgggcgacagagtgtcccggaacaacttcctgggattccctgccagcgaactgcaggccagaatccggacctacaaccagcactacaacaacctgctgagaggcgccgtgtctcagcggctgtatattctgctgcctctggattgcggcgtgcccgacaatctgtctatggccgatcctaatatccggttcctggacaagctgccccagcagacaggcgatcacgccggcattaaggaccgggtgtacagcaacagcatctacgagctgctggaaaacggccagcgagccggaacatgcgtgctggaatatgccacacctctgcagaccctgttcgccatgagccagtatagccaggccggcttcagcagagaggacagactggaacaggccaagctgttctgccggacactggaagatatcctggccgacgctcctgagagccagaacaactgtagactgatcgcctaccaagagcctgccgacgacagcagctttagcctgtctcaagaggtgctgcggcacctgagacaagaggaaaaagaggaagtcaccgtcggcagcctgaaaacctctgccgtgcctagcaccagcaccatgagtcaagaacctgagctgctgatctccggcatggaaaagcccctgcctctgagaaccgacttcagctga

SEQ ID NO: 90 - RNA, 696 nt, Artificial Sequence
  Codon optimised RNA encoding STING-beta
augacaugggugucccugcugaaucaagugggcgacagagugucccggaacaacuuccugggauucccugccagcgaacugcaggccagaauccggaccuacaaccagcacuacaacaaccugcugagaggcgccgugucucagcggcuguauauucugcugccucuggauugcggcgugcccgacaaucugucuauggccgauccuaauauccgguuccuggacaagcugccccagcagacaggcgaucacgccggcauuaaggaccggguguacagcaacagcaucuacgagcugcuggaaaacggccagcgagccggaacaugcgugcuggaauaugccacaccucugcagacccuguucgccaugagccaguauagccaggccggcuucagcagagaggacagacuggaacaggccaagcuguucugccggacacuggaagauauccuggccgacgcuccugagagccagaacaacuguagacugaucgccuaccaagagccugccgacgacagcagcuuuagccugucucaagaggugcugcggcaccugagacaagaggaaaaagaggaagucaccgucggcagccugaaaaccucugccgugccuagcaccagcaccaugagucaagaaccugagcugcugaucuccggcauggaaaagccccugccucugagaaccgacuucagcuga

SEQ ID NO: 91 - protein, 407 aa, Homo sapiens
AQNPMEPSVPQLSLMDVKCETPNCPFFMSVNTQPLCHECSERRQKNQNKLPKLNSKPGPEGLPGMALGASRGEAYEPLAWNPEESTGGPHSAPPTAPSPFLFSETTAMKCRSPGCPFTLNVQHNGFCERCHNARQLHASHAPDHTRHLDPGKCQACLQDVTRTFNGICSTCFKRTTAEASSSLSTSLPPSCHQRSKSDPSRLVRSPSPHSCHRAGNDAPAGCLSQAARTPGDRTGTSKCRKAGCVYFGTPENKGFCTLCFIEYRENKHFAAASGKVSPTASRFQNTIPCLGRECGTLGSTMFEGYCQKCFIEAQNQRFHEAKRTEEQLRSSQRRDVPRTTQSTSRPKCARASCKNILACRSEELCMECQHPNQRMGPGAHRGEPAPEDPPKQRCRAPACDHFGNAKC

SEQ ID NO: 92 - DNA, 1221 bp, Homo sapiens
gcccagaatcccatggaaccttccgtgccccagctttctctcatggatgtaaaatgtgaaacgcccaactgccccttcttcatgtctgtgaacacccagcctttatgccatgagtgctcagagaggcggcaaaagaatcaaaacaaactcccaaagctgaactccaagccgggccctgaggggctccctggcatggcgctcggggcctctcggggagaagcctatgagcccttggcgtggaaccctgaggagtccactggggggcctcattcggccccaccgacagcacccagcccttttctgttcagtgagaccactgccatgaagtgcaggagccccggctgccccttcacactgaatgtgcagcacaacggattttgtgaacgttgccacaacgcccggcaacttcacgccagccacgccccagaccacacaaggcacttggatcccgggaagtgccaagcctgcctccaggatgttaccaggacatttaatgggatctgcagtacttgcttcaaaaggactacagcagaggcctcctccagcctcagcaccagcctccctccttcctgtcaccagcgttccaagtcagatccctcgcggctcgtccggagcccctccccgcattcttgccacagagctggaaacgacgcccctgctggctgcctgtctcaagctgcacggactcctggggacaggacggggacgagcaagtgcagaaaagccggctgcgtgtattttgggactccagaaaacaagggcttttgcacactgtgtttcatcgagtacagagaaaacaaacattttgctgctgcctcagggaaagtcagtcccacagcgtccaggttccagaacaccattccgtgcctggggagggaatgcggcacccttggaagcaccatgtttgaaggatactgccagaagtgtttcattgaagctcagaatcagagatttcatgaggccaaaaggacagaagagcaactgagatcgagccagcgcagagatgtgcctcgaaccacacaaagcacctcaaggcccaagtgcgcccgggcctcctgcaagaacatcctggcctgccgcagcgaggagctctgcatggagtgtcagcatcccaaccagaggatgggccctggggcccaccggggtgagcctgcccccgaagacccccccaagcagcgttgccgggcccccgcctgtgatcattttggcaatgccaagtgc

SEQ ID NO: 93 - RNA, 1221 nt, Homo sapiens
gcccagaaucccauggaaccuuccgugccccagcuuucucucauggauguaaaaugugaaacgcccaacugccccuucuucaugucugugaacacccagccuuuaugccaugagugcucagagaggcggcaaaagaaucaaaacaaacucccaaagcugaacuccaagccgggcccugaggggcucccuggcauggcgcucggggccucucggggagaagccuaugagcccuuggcguggaacccugaggaguccacuggggggccucauucggccccaccgacagcacccagcccuuuucuguucagugagaccacugccaugaagugcaggagccccggcugccccuucacacugaaugugcagcacaacggauuuugugaacguugccacaacgcccggcaacuucacgccagccacgccccagaccacacaaggcacuuggaucccgggaagugccaagccugccuccaggauguuaccaggacauuuaaugggaucugcaguacuugcuucaaaaggacuacagcagaggccuccuccagccucagcaccagccucccuccuuccugucaccagcguuccaagucagaucccucgcggcucguccggagccccuccccgcauucuugccacagagcuggaaacgacgccccugcuggcugccugucucaagcugcacggacuccuggggacaggacggggacgagcaagugcagaaaagccggcugcguguauuuugggacuccagaaaacaagggcuuuugcacacuguguuucaucgaguacagagaaaacaaacauuuugcugcugccucagggaaagucagucccacagcguccagguuccagaacaccauuccgugccuggggagggaaugcggcacccuuggaagcaccauguuugaaggauacugccagaaguguuucauugaagcucagaaucagagauuucaugaggccaaaaggacagaagagcaacugagaucgagccagcgcagagaugugccucgaaccacacaaagcaccucaaggcccaagugcgcccgggccuccugcaagaacauccuggccugccgcagcgaggagcucugcauggagugucagcaucccaaccagaggaugggcccuggggcccaccggggugagccugcccccgaagacccccccaagcagcguugccgggcccccgccugugaucauuuuggcaaugccaagugc

SEQ ID NO: 94 - DNA, 1227 bp, Artificial Sequence
  Codon optimised DNA encoding A20 or TNFAIP3
atggcccagaatcctatggaacctagcgtgccccagctgagcctgatggacgtgaagtgcgaaacccctaactgccccttcttcatgtccgtgaacacccagcctctgtgccacgagtgtagcgagcggagacagaagaaccagaacaagctgcccaagctgaacagcaagcccggacctgaaggactgcctggaatggctctgggagcttctagaggcgaggcctatgaacccctggcctggaatcctgaggaaagcacaggcggacctcacagcgctcctccaacagcaccttctccatttctgttcagcgagacaaccgccatgaagtgcagaagccctggctgccctttcacactgaacgtgcagcacaacggcttttgcgagagatgccacaacgccagacagctgcacgcttctcacgcccctgatcacaccagacacctggatcctggaaagtgccaggcctgcctgcaggatgtgaccagaaccttcaacggcatctgcagcacctgtttcaagcggacaacagccgaggccagcagcagcctgtctacatctctgcctccaagctgccaccagcggagcaagagcgatccttctagacttgtgcggagcccctctcctcactcctgtcacagagccggaaatgatgcccctgccggatgtctgtctcaggccgctagaacacctggcgatagaaccggcaccagcaagtgtagaaaggccggctgcgtgtacttcggcacccctgagaacaagggattctgcaccctgtgcttcatcgagtacagagagaacaagcacttcgccgctgcctccggaaaggtgtcacctaccgctagccggttccagaacacaatcccttgcctgggcagagagtgtggcacactgggcagcacaatgttcgagggctactgccagaagtgctttatcgaggcccagaaccagcggttccacgaggccaagagaaccgaggaacagctgagaagcagccagagaagggacgtgcccagaacaacccagagcaccagcagacctaagtgcgccagagccagctgcaagaacatcctggcctgtcggagcgaggaactgtgcatggaatgccagcatcctaaccagagaatgggccctggcgctcacagaggcgaacctgctccagaagatcctcctaagcagcggtgtagagcccctgcctgtgaccactttggcaacgccaagtgctga

SEQ ID NO: 95 - RNA, 1227 nt, Artificial Sequence
  Codon optimised RNA encoding A20 or TNFAIP3
auggcccagaauccuauggaaccuagcgugccccagcugagccugauggacgugaagugcgaaaccccuaacugccccuucuucauguccgugaacacccagccucugugccacgaguguagcgagcggagacagaagaaccagaacaagcugcccaagcugaacagcaagcccggaccugaaggacugccuggaauggcucugggagcuucuagaggcgaggccuaugaaccccuggccuggaauccugaggaaagcacaggcggaccucacagcgcuccuccaacagcaccuucuccauuucuguucagcgagacaaccgccaugaagugcagaagcccuggcugcccuuucacacugaacgugcagcacaacggcuuuugcgagagaugccacaacgccagacagcugcacgcuucucacgccccugaucacaccagacaccuggauccuggaaagugccaggccugccugcaggaugugaccagaaccuucaacggcaucugcagcaccuguuucaagcggacaacagccgaggccagcagcagccugucuacaucucugccuccaagcugccaccagcggagcaagagcgauccuucuagacuugugcggagccccucuccucacuccugucacagagccggaaaugaugccccugccggaugucugucucaggccgcuagaacaccuggcgauagaaccggcaccagcaaguguagaaaggccggcugcguguacuucggcaccccugagaacaagggauucugcacccugugcuucaucgaguacagagagaacaagcacuucgccgcugccuccggaaaggugucaccuaccgcuagccgguuccagaacacaaucccuugccugggcagagaguguggcacacugggcagcacaauguucgagggcuacugccagaagugcuuuaucgaggcccagaaccagcgguuccacgaggccaagagaaccgaggaacagcugagaagcagccagagaagggacgugcccagaacaacccagagcaccagcagaccuaagugcgccagagccagcugcaagaacauccuggccugucggagcgaggaacugugcauggaaugccagcauccuaaccagagaaugggcccuggcgcucacagaggcgaaccugcuccagaagauccuccuaagcagcgguguagagccccugccugugaccacuuuggcaacgccaagugcuga

SEQ ID NO: 96 - protein, 185 aa, Homo sapiens
KCRKAGCVYFGTPENKGFCTLCFIEYRENKHFAAASGKVSPTASRFQNTIPCLGRECGTLGSTMFEGYCQKCFIEAQNQRFHEAKRTEEQLRSSQRRDVPRTTQSTSRPKCARASCKNILACRSEELCMECQHPNQRMGPGAHRGEPAPEDPPKQRCRAPACDHFGNAKCNGYCNECFQFKQMYG

SEQ ID NO: 97 - DNA, 555 bp, Homo sapiens
aagtgcagaaaagccggctgcgtgtattttgggactccagaaaacaagggcttttgcacactgtgtttcatcgagtacagagaaaacaaacattttgctgctgcctcagggaaagtcagtcccacagcgtccaggttccagaacaccattccgtgcctggggagggaatgcggcacccttggaagcaccatgtttgaaggatactgccagaagtgtttcattgaagctcagaatcagagatttcatgaggccaaaaggacagaagagcaactgagatcgagccagcgcagagatgtgcctcgaaccacacaaagcacctcaaggcccaagtgcgcccgggcctcctgcaagaacatcctggcctgccgcagcgaggagctctgcatggagtgtcagcatcccaaccagaggatgggccctggggcccaccggggtgagcctgcccccgaagacccccccaagcagcgttgccgggcccccgcctgtgatcattttggcaatgccaagtgcaacggctactgcaacgaatgctttcagttcaagcagatgtatggc

SEQ ID NO: 98 - RNA, 555 nt, Homo sapiens
aagugcagaaaagccggcugcguguauuuugggacuccagaaaacaagggcuuuugcacacuguguuucaucgaguacagagaaaacaaacauuuugcugcugccucagggaaagucagucccacagcguccagguuccagaacaccauuccgugccuggggagggaaugcggcacccuuggaagcaccauguuugaaggauacugccagaaguguuucauugaagcucagaaucagagauuucaugaggccaaaaggacagaagagcaacugagaucgagccagcgcagagaugugccucgaaccacacaaagcaccucaaggcccaagugcgcccgggccuccugcaagaacauccuggccugccgcagcgaggagcucugcauggagugucagcaucccaaccagaggaugggcccuggggcccaccggggugagccugcccccgaagacccccccaagcagcguugccgggcccccgccugugaucauuuuggcaaugccaagugcaacggcuacugcaacgaaugcuuucaguucaagcagauguauggc

SEQ ID NO: 99 - DNA, 561 bp, Artificial Sequence
  Codon optimised DNA encoding A20
atgaagtgcagaaaggccggctgcgtgtacttcggcacccctgagaacaagggcttctgcaccctgtgcttcatcgagtacagagagaacaagcacttcgctgccgccagcggaaaggtgtcacctaccgccagcagattccagaacacaatcccctgcctgggcagagagtgtggcacactgggcagcacaatgttcgagggctactgccagaagtgctttatcgaggcccagaaccagcggttccacgaggccaagagaaccgaggaacagctgagaagcagccagagaagggacgtgcccagaacaacccagagcaccagcagacctaagtgcgccagagccagctgcaagaacatcctggcctgcagatccgaggaactgtgcatggaatgccagcatcctaaccagagaatgggccctggcgctcacagaggcgaacctgctccagaagatcctcctaagcagcggtgtagagccccagcctgtgaccactttggcaacgccaagtgcaacggctactgcaacgagtgcttccagttcaagcagatgtacggctga

SEQ ID NO: 100 - RNA, 561 nt, Artificial Sequence
  Codon optimised RNA encoding A20
augaagugcagaaaggccggcugcguguacuucggcaccccugagaacaagggcuucugcacccugugcuucaucgaguacagagagaacaagcacuucgcugccgccagcggaaaggugucaccuaccgccagcagauuccagaacacaauccccugccugggcagagaguguggcacacugggcagcacaauguucgagggcuacugccagaagugcuuuaucgaggcccagaaccagcgguuccacgaggccaagagaaccgaggaacagcugagaagcagccagagaagggacgugcccagaacaacccagagcaccagcagaccuaagugcgccagagccagcugcaagaacauccuggccugcagauccgaggaacugugcauggaaugccagcauccuaaccagagaaugggcccuggcgcucacagaggcgaaccugcuccagaagauccuccuaagcagcgguguagagccccagccugugaccacuuuggcaacgccaagugcaacggcuacugcaacgagugcuuccaguucaagcagauguacggcuga

SEQ ID NO: 101 - protein, 131 aa, Homo sapiens
EAVRLIMDSLHMAAREQQVYCEEMREERQDRLKFIDKQLELLAQDYKLRIKQITEEVERQVSTAMAEEIRRLSVLVDDYQMDFHPSPVVLKVYKNELHRHIEEGLGRNMSDRCSTAITNSLQTMQQDMIDG

SEQ ID NO: 102 - DNA, 393 bp, Homo sapiens
gaggcggttcgactcatcatggactccctgcacatggcggctcgggagcagcaggtttactgcgaggaaatgcgtgaagagcggcaagaccgactgaaatttattgacaaacagctggagctcttggctcaagactataagctgcgaattaagcagattacggaggaagtggagaggcaggtgtcgactgcaatggccgaggagatcaggcgcctctctgtactggtggacgattaccagatggacttccacccttctccagtagtcctcaaggtttataagaatgagctgcaccgccacatagaggaaggactgggtcgaaacatgtctgaccgctgctccacggccatcaccaactccctgcagaccatgcagcaggacatgatagatggc

SEQ ID NO: 103 - RNA, 393 nt, Homo sapiens
gaggcgguucgacucaucauggacucccugcacauggcggcucgggagcagcagguuuacugcgaggaaaugcgugaagagcggcaagaccgacugaaauuuauugacaaacagcuggagcucuuggcucaagacuauaagcugcgaauuaagcagauuacggaggaaguggagaggcaggugucgacugcaauggccgaggagaucaggcgccucucuguacugguggacgauuaccagauggacuuccacccuucuccaguaguccucaagguuuauaagaaugagcugcaccgccacauagaggaaggacugggucgaaacaugucugaccgcugcuccacggccaucaccaacucccugcagaccaugcagcaggacaugauagauggc

SEQ ID NO: 104 - DNA, 399 bp, Artificial Sequence
  Codon optimised DNA encoding MFN2 protein
atggaagccgtgcggctgatcatggacagcctgcatatggccgccagagagcagcaggtctactgcgaggaaatgcgggaagagagacaggaccggctgaagttcatcgacaagcagctggaactgctggcccaggactacaagctgcggatcaagcagatcaccgaagaggtggaaagacaggtgtccaccgccatggccgaggaaatcagacgactgagcgtgctggtggacgactaccagatggactttcacccctctccagtggtgctgaaggtgtacaagaacgagctgcaccggcacatcgaggaaggcctgggcagaaacatgagcgacagatgcagcaccgccatcaccaatagcctgcagaccatgcagcaggacatgatcgacggctga

SEQ ID NO: 105 - RNA, 399 nt, Artificial Sequence
  Codon optimised RNA encoding MFN2
auggaagccgugcggcugaucauggacagccugcauauggccgccagagagcagcaggucuacugcgaggaaaugcgggaagagagacaggaccggcugaaguucaucgacaagcagcuggaacugcuggcccaggacuacaagcugcggaucaagcagaucaccgaagagguggaaagacagguguccaccgccauggccgaggaaaucagacgacugagcgugcugguggacgacuaccagauggacuuucaccccucuccaguggugcugaagguguacaagaacgagcugcaccggcacaucgaggaaggccugggcagaaacaugagcgacagaugcagcaccgccaucaccaauagccugcagaccaugcagcaggacaugaucgacggcuga

SEQ ID NO: 106 - protein, 81 aa, Homo sapiens
RLKFIDKQLELLAQDYKLRIKQITEEVERQVSTAMAEEIRRLSVLVDDYQMDFHPSPVVLKVYKNELHRHIEEGLGRNMSD

SEQ ID NO: 107 - DNA, 243 bp, Homo sapiens
cgactgaaatttattgacaaacagctggagctcttggctcaagactataagctgcgaattaagcagattacggaggaagtggagaggcaggtgtcgactgcaatggccgaggagatcaggcgcctctctgtactggtggacgattaccagatggacttccacccttctccagtagtcctcaaggtttataagaatgagctgcaccgccacatagaggaaggactgggtcgaaacatgtctgac

SEQ ID NO: 108 - RNA, 243 nt, Homo sapiens
cgacugaaauuuauugacaaacagcuggagcucuuggcucaagacuauaagcugcgaauuaagcagauuacggaggaaguggagaggcaggugucgacugcaauggccgaggagaucaggcgccucucuguacugguggacgauuaccagauggacuuccacccuucuccaguaguccucaagguuuauaagaaugagcugcaccgccacauagaggaaggacugggucgaaacaugucugac

SEQ ID NO: 109 - DNA, 249 bp, Artificial Sequence
  Codon optimised DNA encoding MFN2
atgcggctgaagttcatcgacaagcagctggaactgctggcccaggactacaagctgcggatcaagcagatcaccgaagaggtggaaagacaggtgtccaccgccatggccgaggaaatcagacgactgagcgtgctggtggacgactaccagatggactttcacccctctccagtggtgctgaaggtgtacaagaacgagctgcaccggcacatcgaggaaggcctgggcagaaacatgagcgactga

SEQ ID NO: 110 - RNA, 249 nt, Artificial Sequence
  Codon optimised RNA encoding MFN2
augcggcugaaguucaucgacaagcagcuggaacugcuggcccaggacuacaagcugcggaucaagcagaucaccgaagagguggaaagacagguguccaccgccauggccgaggaaaucagacgacugagcgugcugguggacgacuaccagauggacuuucaccccucuccaguggugcugaagguguacaagaacgagcugcaccggcacaucgaggaaggccugggcagaaacaugagcgacuga

SEQ ID NO: 111 - protein, 234 aa, Homo sapiens
MSEEEQGSGTTTGCGLPSIEQMLAANPGKTPISLLQEYGTRIGKTPVYDLLKAEGQAHQPNFTFRVTVGDTSCTGQGPSKKAAKHKAAEVALKHLKGGSMLEPALEDSSSFSPLDSSLPEDIPVFTAAAAATPVPSVVLTRSPPMELQPPVSPQQSECNPVGALQELVVQKGWRLPEYTVTQESGPAHRKEFTMTCRVERFIEIGSGTSKKLAKRNAAAKMLLRVHTVPLDARD

SEQ ID NO: 112 - DNA, 702 bp, Homo sapiens
atgagtgaagaggagcaaggctccggcactaccacgggctgcgggctgcctagtatagagcaaatgctggccgccaacccaggcaagaccccgatcagccttctgcaggagtatgggaccagaatagggaagacgcctgtgtacgaccttctcaaagccgagggccaagcccaccagcctaatttcaccttccgggtcaccgttggcgacaccagctgcactggtcagggccccagcaagaaggcagccaagcacaaggcagctgaggtggccctcaaacacctcaaaggggggagcatgctggagccggccctggaggacagcagttctttttctcccctagactcttcactgcctgaggacattccggtttttactgctgcagcagctgctaccccagttccatctgtagtcctaaccaggagcccccccatggaactgcagccccctgtctcccctcagcagtctgagtgcaaccccgttggtgctctgcaggagctggtggtgcagaaaggctggcggttgccggagtacacagtgacccaggagtctgggccagcccaccgcaaagaattcaccatgacctgtcgagtggagcgtttcattgagattgggagtggcacttccaaaaaattggcaaagcggaatgcggcggccaaaatgctgcttcgagtgcacacggtgcctctggatgcccgggat

SEQ ID NO: 113 - RNA, 702 nt, Homo sapiens
augagugaagaggagcaaggcuccggcacuaccacgggcugcgggcugccuaguauagagcaaaugcuggccgccaacccaggcaagaccccgaucagccuucugcaggaguaugggaccagaauagggaagacgccuguguacgaccuucucaaagccgagggccaagcccaccagccuaauuucaccuuccgggucaccguuggcgacaccagcugcacuggucagggccccagcaagaaggcagccaagcacaaggcagcugagguggcccucaaacaccucaaaggggggagcaugcuggagccggcccuggaggacagcaguucuuuuucuccccuagacucuucacugccugaggacauuccgguuuuuacugcugcagcagcugcuaccccaguuccaucuguaguccuaaccaggagcccccccauggaacugcagcccccugucuccccucagcagucugagugcaaccccguuggugcucugcaggagcugguggugcagaaaggcuggcgguugccggaguacacagugacccaggagucugggccagcccaccgcaaagaauucaccaugaccugucgaguggagcguuucauugagauugggaguggcacuuccaaaaaauuggcaaagcggaaugcggcggccaaaaugcugcuucgagugcacacggugccucuggaugcccgggau

SEQ ID NO: 114 - DNA, 705 bp, Artificial Sequence
  Codon optimised DNA encoding TRBP
atgagcgaggaagaacaaggcagcggcaccaccacaggatgtggcctgccttctatcgagcagatgctggccgccaatcctggcaagacacctatcagcctgctgcaagagtacggcacccggatcggaaagacccctgtgtacgatctgctgaaggccgaaggccaggctcaccagcctaacttcaccttcagagtgaccgtgggcgacaccagctgtacaggacagggcccttctaagaaggccgccaaacacaaagccgccgaggtggccctgaaacacctgaaaggcggctccatgctggaacccgctctggaagatagcagcagcttcagccctctggacagcagcctgcctgaggacatccctgtgtttacagccgctgccgctgctacacctgtgccatctgtggtgctgaccagatctcctccaatggaactgcagcctcctgtgtctcctcagcagagcgagtgtaatcctgtgggcgccctgcaagaactggtggtgcaaaaaggatggcggctgcccgagtacaccgtgacacaagaatctggccccgctcaccggaaagaattcaccatgacctgcagagtggaacggttcatcgagatcggctccggcacctctaagaagctggccaagagaaacgccgctgccaagatgctgctgcgggtgcacacagttcctctggacgccagagattga

SEQ ID NO: 115 - RNA, 705 nt, Artificial Sequence
  Codon optimised RNA encoding TRBP
augagcgaggaagaacaaggcagcggcaccaccacaggauguggccugccuucuaucgagcagaugcuggccgccaauccuggcaagacaccuaucagccugcugcaagaguacggcacccggaucggaaagaccccuguguacgaucugcugaaggccgaaggccaggcucaccagccuaacuucaccuucagagugaccgugggcgacaccagcuguacaggacagggcccuucuaagaaggccgccaaacacaaagccgccgagguggcccugaaacaccugaaaggcggcuccaugcuggaacccgcucuggaagauagcagcagcuucagcccucuggacagcagccugccugaggacaucccuguguuuacagccgcugccgcugcuacaccugugccaucuguggugcugaccagaucuccuccaauggaacugcagccuccugugucuccucagcagagcgaguguaauccugugggcgcccugcaagaacugguggugcaaaaaggauggcggcugcccgaguacaccgugacacaagaaucuggccccgcucaccggaaagaauucaccaugaccugcagaguggaacgguucaucgagaucggcuccggcaccucuaagaagcuggccaagagaaacgccgcugccaagaugcugcugcgggugcacacaguuccucuggacgccagagauuga

SEQ ID NO: 116 - protein, 200 aa, Homo sapiens
MADPEVCCFITKILCAHGGRMALDALLQEIALSEPQLCEVLQVAGPDRFVVLETGGEAGITRSVVATTRARVCRRKYCQRPCDNLHLCKLNLLGRCNYSQSERNLCKYSHEVLSEENFKVLKNHELSGLNKEELAVLLLQSDPFFMPEICKSYKGEGRQQICNQQPPCSRLHICDHFTRGNCRFPNCLRSHNLMDRKVLA

SEQ ID NO: 117 - DNA, 600 bp, Homo sapiens
atggcggacccggaggtgtgctgcttcatcaccaaaatcctgtgcgcccacgggggccgcatggccctggacgcgctgctccaggagatcgcgctgtctgagccgcagctctgtgaggtgctgcaggtggccgggcccgaccgctttgtggtgttggagaccggcggcgaggccgggatcacccgatcggtggtggccaccactcgagcccgggtctgccgtcgcaagtactgccagagaccctgcgataacctgcatctctgcaaactcaacttgctgggccggtgcaactattcgcagtccgagcggaatttatgcaaatattctcatgaggttctctcagaagagaacttcaaagtcctgaaaaatcacgaactctctggactgaacaaagaggaattagcagtgctcctcctccaaagtgatcctttttttatgcccgagatatgcaaaagttataagggagagggtcggcagcagatttgtaaccagcagccaccgtgttcaagactccacatctgtgaccacttcacccgagggaactgtcgttttcccaactgcctccggtcccataacctgatggacagaaaggtgctggcc

SEQ ID NO: 118 - RNA, 600 nt, Homo sapiens
auggcggacccggaggugugcugcuucaucaccaaaauccugugcgcccacgggggccgcauggcccuggacgcgcugcuccaggagaucgcgcugucugagccgcagcucugugaggugcugcagguggccgggcccgaccgcuuugugguguuggagaccggcggcgaggccgggaucacccgaucggugguggccaccacucgagcccgggucugccgucgcaaguacugccagagacccugcgauaaccugcaucucugcaaacucaacuugcugggccggugcaacuauucgcaguccgagcggaauuuaugcaaauauucucaugagguucucucagaagagaacuucaaaguccugaaaaaucacgaacucucuggacugaacaaagaggaauuagcagugcuccuccuccaaagugauccuuuuuuuaugcccgagauaugcaaaaguuauaagggagagggucggcagcagauuuguaaccagcagccaccguguucaagacuccacaucugugaccacuucacccgagggaacugucguuuucccaacugccuccggucccauaaccugauggacagaaaggugcuggcc

SEQ ID NO: 119 - DNA, 603 bp, Artificial Sequence
  Codon optimised DNA encoding Zinc finger anti-viral protein
atggccgatcctgaagtgtgctgcttcatcaccaagatcctgtgcgcccacggcggaagaatggctctggatgctctgctgcaagagatcgccctgtctgagcctcagctgtgcgaagtgctgcaagtggccggacctgacagattcgtggtgctggaaacaggcggagaggccggcattaccagatccgtggtggctaccacaagagccagagtgtgccggcggaagtactgccagaggccttgcgataatctgcacctgtgcaagctgaacctgctgggcagatgcaactacagccagagcgagcggaatctgtgcaagtactcccacgaggtgctgagcgaagagaacttcaaggtgctgaagaaccacgagctgagcggcctgaacaaagaggaactggccgttctgctgctgcagagcgacccattcttcatgcccgagatctgcaagagctacaaaggcgagggcagacagcagatctgtaaccagcagcctccatgcagcagactgcacatctgcgaccacttcacccggggcaactgcagattccccaactgcctgagaagccacaacctgatggaccggaaggtgctggcttga

SEQ ID NO: 120 - RNA, 603 nt, Artificial Sequence
  Codon optimised RNA encoding Zinc finger anti-viral protein
auggccgauccugaagugugcugcuucaucaccaagauccugugcgcccacggcggaagaauggcucuggaugcucugcugcaagagaucgcccugucugagccucagcugugcgaagugcugcaaguggccggaccugacagauucguggugcuggaaacaggcggagaggccggcauuaccagauccgugguggcuaccacaagagccagagugugccggcggaaguacugccagaggccuugcgauaaucugcaccugugcaagcugaaccugcugggcagaugcaacuacagccagagcgagcggaaucugugcaaguacucccacgaggugcugagcgaagagaacuucaaggugcugaagaaccacgagcugagcggccugaacaaagaggaacuggccguucugcugcugcagagcgacccauucuucaugcccgagaucugcaagagcuacaaaggcgagggcagacagcagaucuguaaccagcagccuccaugcagcagacugcacaucugcgaccacuucacccggggcaacugcagauuccccaacugccugagaagccacaaccugauggaccggaaggugcuggcuuga

SEQ ID NO: 121 - protein, 170 aa, Homo sapiens
MAGDLSAGFFMEELNTYRQKQGVVLKYQELPNSGPPHDRRFTFQVIIDGREFPEGEGRSKKEAKNAAAKLAVEILNKEKKAVSPLLLTTTNSSEGLSMGNYIGLINRIAQKKRLTVNYEQCASGVHGPEGFHYKCKMGQKEYSIGTGSTKQEAKQLAAKLAYLQILSEET

SEQ ID NO: 122 - DNA, 510 bp, Homo sapiens
atggctggtgatctttcagcaggtttcttcatggaggaacttaatacataccgtcagaagcagggagtagtacttaaatatcaagaactgcctaattcaggacctccacatgataggaggtttacatttcaagttataatagatggaagagaatttccagaaggtgaaggtagatcaaagaaggaagcaaaaaatgccgcagccaaattagctgttgagatacttaataaggaaaagaaggcagttagtcctttattattgacaacaacgaattcttcagaaggattatccatggggaattacataggccttatcaatagaattgcccagaagaaaagactaactgtaaattatgaacagtgtgcatcgggggtgcatgggccagaaggatttcattataaatgcaaaatgggacagaaagaatatagtattggtacaggttctactaaacaggaagcaaaacaattggccgctaaacttgcatatcttcagatattatcagaagaaacc

SEQ ID NO: 123 - RNA, 510 nt, Homo sapiens
auggcuggugaucuuucagcagguuucuucauggaggaacuuaauacauaccgucagaagcagggaguaguacuuaaauaucaagaacugccuaauucaggaccuccacaugauaggagguuuacauuucaaguuauaauagauggaagagaauuuccagaaggugaagguagaucaaagaaggaagcaaaaaaugccgcagccaaauuagcuguugagauacuuaauaaggaaaagaaggcaguuaguccuuuauuauugacaacaacgaauucuucagaaggauuauccauggggaauuacauaggccuuaucaauagaauugcccagaagaaaagacuaacuguaaauuaugaacagugugcaucgggggugcaugggccagaaggauuucauuauaaaugcaaaaugggacagaaagaauauaguauugguacagguucuacuaaacaggaagcaaaacaauuggccgcuaaacuugcauaucuucagauauuaucagaagaaacc

SEQ ID NO: 124 - DNA, 513 bp, Artificial Sequence
  Codon optimised DNA encoding PKR
atggctggcgatctgagcgccggcttcttcatggaagaactgaacacctaccggcagaaacagggcgtcgtgctgaagtaccaagagctgcctaatagcggccctcctcacgaccggcggttcacctttcaagtgatcatcgacggcagagagttccccgaaggcgagggcagatctaagaaagaggccaagaacgccgctgccaagctggccgtggaaatcctgaacaaagagaagaaggccgtttctcccctgctgctgaccaccaccaatagctctgagggcctgagcatgggcaactacatcggcctgatcaaccggatcgcccagaaaaagcggctgaccgtgaactacgagcagtgtgccagcggagtgcacggccctgagggctttcactacaagtgcaagatgggccagaaagagtacagcatcggcaccggcagcaccaagcaagaagccaaacagctggccgccaaactggcctacctgcagatcctgagcgaggaaacctga

SEQ ID NO: 125 - RNA, 513 nt, Artificial Sequence
  Codon optimised RNA encoding PKR
auggcuggcgaucugagcgccggcuucuucauggaagaacugaacaccuaccggcagaaacagggcgucgugcugaaguaccaagagcugccuaauagcggcccuccucacgaccggcgguucaccuuucaagugaucaucgacggcagagaguuccccgaaggcgagggcagaucuaagaaagaggccaagaacgccgcugccaagcuggccguggaaauccugaacaaagagaagaaggccguuucuccccugcugcugaccaccaccaauagcucugagggccugagcaugggcaacuacaucggccugaucaaccggaucgcccagaaaaagcggcugaccgugaacuacgagcagugugccagcggagugcacggcccugagggcuuucacuacaagugcaagaugggccagaaagaguacagcaucggcaccggcagcaccaagcaagaagccaaacagcuggccgccaaacuggccuaccugcagauccugagcgaggaaaccuga

SEQ ID NO: 126 - protein, 194 aa, Homo sapiens
MSQSRHRAEAPPLEREDSGTFSLGKMITAKPGKTPIQVLHEYGMKTKNIPVYECERSDVQIHVPTFTFRVTVGDITCTGEGTSKKLAKHRAAEAAINILKANASICFAVPDPLMPDPSKQPKNQLNPIGSLQELAIHHGWRLPEYTLSQEGGPAHKREYTTICRLESFMETGKGASKKQAKRNAAEKFLAKFSN

SEQ ID NO: 127 - DNA, 582 bp, Homo sapiens
atgtcccagagcaggcaccgcgccgaggccccgccgctggagcgcgaggacagtgggaccttcagtttggggaagatgataacagctaagccagggaaaacaccgattcaggtattacacgaatacggcatgaagaccaagaacatcccagtttatgaatgtgaaagatctgatgtgcaaatacacgtgcccactttcaccttcagagtaaccgttggtgacataacctgcacaggtgaaggtacaagtaagaagctggcgaaacatagagctgcagaggctgccataaacattttgaaagccaatgcaagtatttgctttgcagttcctgaccccttaatgcctgacccttccaagcaaccaaagaaccagcttaatcctattggttcattacaggaattggctattcatcatggctggagacttcctgaatataccctttcccaggagggaggacctgctcataagagagaatatactacaatttgcaggctagagtcatttatggaaactggaaagggggcatcaaaaaagcaagccaaaaggaatgctgctgagaaatttcttgccaaatttagtaat

SEQ ID NO: 128 - RNA, 582 nt, Homo sapiens
augucccagagcaggcaccgcgccgaggccccgccgcuggagcgcgaggacagugggaccuucaguuuggggaagaugauaacagcuaagccagggaaaacaccgauucagguauuacacgaauacggcaugaagaccaagaacaucccaguuuaugaaugugaaagaucugaugugcaaauacacgugcccacuuucaccuucagaguaaccguuggugacauaaccugcacaggugaagguacaaguaagaagcuggcgaaacauagagcugcagaggcugccauaaacauuuugaaagccaaugcaaguauuugcuuugcaguuccugaccccuuaaugccugacccuuccaagcaaccaaagaaccagcuuaauccuauugguucauuacaggaauuggcuauucaucauggcuggagacuuccugaauauacccuuucccaggagggaggaccugcucauaagagagaauauacuacaauuugcaggcuagagucauuuauggaaacuggaaagggggcaucaaaaaagcaagccaaaaggaaugcugcugagaaauuucuugccaaauuuaguaau

SEQ ID NO: 129 - DNA, 513 bp, Artificial Sequence; identical to SEQ ID NO: 124
  Codon optimised DNA encoding PACT

SEQ ID NO: 130 - RNA, 585 nt, Artificial Sequence
  Codon optimised RNA encoding PACT
augagccagagcagacacagagccgaagcuccuccacuggaaagagaggacagcggcaccuuuagccugggcaagaugaucacagccaagccuggcaagaccccuauccaggugcugcacgaguacggcaugaagaccaagaacauccccguguacgagugcgagagaagcgacgugcagauccacgugccaaccuucaccuucagagugaccgugggcgacaucaccuguaccggcgagggcacaucuaagaagcuggccaaacauagagccgccgaggccgccaucaauauccugaaggccaaugccagcaucugcuucgccgugccugauccucugaugcccgauccuagcaagcagcccaagaaccagcugaacccuaucggcagccugcaagagcuggccauucaucauggauggcggcugccugaguacacccugucucaagaaggcggcccugcucacaagagagaguacaccaccaucugccggcuggaaagcuucauggaaacaggcaagggcgccagcaagaaacaggccaagagaaacgccgccgagaaguuccuggccaaguucagcaacuga

SEQ ID NO: 131 - protein, 330 aa, Homo sapiens
MESRDHNNPQEGPTSSSGRRAAVEDNHLLIKAVQNEDVDLVQQLLEGGANVNFQEEEGGWTPLHNAVQMSREDIVELLLRHGADPVLRKKNGATPFILAAIAGSVKLLKLFLSKGADVNECDFYGFTAFMEAAVYGKVKALKFLYKRGANVNLRRKTKEDQERLRKGGATALMDAAEKGHVEVLKILLDEMGADVNACDNMGRNALIHALLSSDDSDVEAITHLLLDHGADVNVRGERGKTPLILAVEKKHLGLVQRLLEQEHIEINDTDSDGKTALLLAVELKLKKIAELLCKRGASTDCGDLVMTARRNYDHSLVKVLLSHGAKEDFH

SEQ ID NO: 132 - DNA, 990 bp, Homo sapiens
atggagagcagggatcataacaacccccaggagggacccacgtcctccagcggtagaagggctgcagtggaagacaatcacttgctgattaaagctgttcaaaacgaagatgttgacctggtccagcaattgctggaaggtggagccaatgttaatttccaggaagaggaagggggctggacacctctgcataacgcagtacaaatgagcagggaggacattgtggaacttctgcttcgtcatggtgctgaccctgttctgaggaagaagaatggggccacgccttttatcctcgcagcgattgcggggagcgtgaagctgctgaaacttttcctttctaaaggagcagatgtcaatgagtgtgatttttatggcttcacagccttcatggaagccgctgtgtatggtaaggtcaaagccctaaaattcctttataagagaggagcaaatgtgaatttgaggcgaaagacaaaggaggatcaagagcggctgaggaaaggaggggccacagctctcatggacgctgctgaaaaaggacacgtagaggtcttgaagattctccttgatgagatgggggcagatgtaaacgcctgtgacaatatgggcagaaatgccttgatccatgctctcctgagctctgacgatagtgatgtggaggctattacgcatctgctgctggaccatggggctgatgtcaatgtgaggggagaaagagggaagactcccctgatcctggcagtggagaagaagcacttgggtttggtgcagaggcttctggagcaagagcacatagagattaatgacacagacagtgatggcaaaacagcactgctgcttgctgttgaactcaaactgaagaaaatcgccgagttgctgtgcaaacgtggagccagtacagattgtggggatcttgttatgacagcgaggcggaattatgaccattcccttgtgaaggttcttctctctcatggagccaaagaagattttcac

SEQ ID NO: 133 - RNA, 990 nt, Homo sapiens
auggagagcagggaucauaacaacccccaggagggacccacguccuccagcgguagaagggcugcaguggaagacaaucacuugcugauuaaagcuguucaaaacgaagauguugaccugguccagcaauugcuggaagguggagccaauguuaauuuccaggaagaggaagggggcuggacaccucugcauaacgcaguacaaaugagcagggaggacauuguggaacuucugcuucgucauggugcugacccuguucugaggaagaagaauggggccacgccuuuuauccucgcagcgauugcggggagcgugaagcugcugaaacuuuuccuuucuaaaggagcagaugucaaugagugugauuuuuauggcuucacagccuucauggaagccgcuguguaugguaaggucaaagcccuaaaauuccuuuauaagagaggagcaaaugugaauuugaggcgaaagacaaaggaggaucaagagcggcugaggaaaggaggggccacagcucucauggacgcugcugaaaaaggacacguagaggucuugaagauucuccuugaugagaugggggcagauguaaacgccugugacaauaugggcagaaaugccuugauccaugcucuccugagcucugacgauagugauguggaggcuauuacgcaucugcugcuggaccauggggcugaugucaaugugaggggagaaagagggaagacuccccugauccuggcaguggagaagaagcacuuggguuuggugcagaggcuucuggagcaagagcacauagagauuaaugacacagacagugauggcaaaacagcacugcugcuugcuguugaacucaaacugaagaaaaucgccgaguugcugugcaaacguggagccaguacagauuguggggaucuuguuaugacagcgaggcggaauuaugaccauucccuugugaagguucuucucucucauggagccaaagaagauuuucac

SEQ ID NO: 134 - DNA, 993 bp, Artificial Sequence
  Codon optimised DNA encoding RNAse L
atggaaagccgggaccacaacaaccctcaagagggccctacaagcagctctggtagaagggccgctgtggaagataaccatctgctgatcaaggccgtgcagaacgaggacgtggacctggtgcaacaactgctggaaggcggagccaacgtgaacttccaagaggaagaaggcggctggacccctctgcataacgctgtgcagatgagcagagaggacatcgtcgagctgctgctgagacatggcgctgaccctgtgctgagaaagaagaacggcgccacacctttcatcctggccgccattgccggaagcgtgaagctgctgaagctgttcctgagcaagggcgccgatgtgaacgagtgcgacttctacggcttcaccgccttcatggaagccgccgtgtacggcaaagtgaaggccctgaagttcctgtacaagaggggcgctaacgtgaacctgcggagaaagaccaaagaggaccaagagcggctgcggaaaggtggcgctacagctcttatggatgccgccgagaagggacacgtggaagtgctgaagatcctgctggatgagatgggcgcagacgtgaacgcctgcgacaacatgggaagaaacgccctgattcacgccctgctgagcagcgacgatagcgacgtggaagccatcacacatctgctgctggatcacggggctgatgtgaatgtgcggggcgagagaggaaagaccccactgattctggccgtggaaaagaaacacctgggcctcgtgcagaggctgctggaacaagagcacatcgagatcaacgacaccgacagcgacggcaagacagccctgctgcttgccgtggaactgaagctgaagaagatcgccgaactgctgtgcaagagaggcgccagcacagattgtggcgacctcgtgatgaccgccagacggaactacgatcacagcctggtcaaggtgctgctgtcccatggcgctaaagaggacttccactga

SEQ ID NO: 135 - RNA, 990 nt, Artificial Sequence; identical to SEQ ID NO: 133
  Codon optimised RNA encoding RNAse L

SEQ ID NO: 136 - protein, 343 aa, Homo sapiens
MDLYSTPAAALDRFVARRLQPRKEFVEKARRALGALAAALRERGGRLGAAAPRVLKTVKGGSSGRGTALKGGCDSELVIFLDCFKSYVDQRARRAEILSEMRASLESWWQNPVPGLRLTFPEQSVPGALQFRLTSVDLEDWMDVSLVPAFNVLGQAGSGVKPKPQVYSTLLNSGCQGGEHAACFTELRRNFVNIRPAKLKNLILLVKHWYHQVCLQGLWKETLPPVYALELLTIFAWEQGCKKDAFSLAEGLRTVLGLIQQHQHLCVFWTVNYGFEDPAVGQFLQRQLKRPRPVILDPADPTWDLGNGAAWHWDLLAQEAASCYDHPCFLRGMGDPVQSWKGP

SEQ ID NO: 137 - DNA, 1029 bp, Homo sapiens
atggacttgtacagcaccccggccgctgcgctggacaggttcgtggccagaaggctgcagccgcggaaggagttcgtagagaaggcgcggcgcgctctgggcgccctggccgctgccctgagggagcgcgggggccgcctcggtgctgctgccccgcgggtgctgaaaactgtcaagggaggctcctcgggccggggcacagctctcaagggtggctgtgattctgaacttgtcatcttcctcgactgcttcaagagctatgtggaccagagggcccgccgtgcagagatcctcagtgagatgcgggcatcgctggaatcctggtggcagaacccagtccctggtctgagactcacgtttcctgagcagagcgtgcctggggccctgcagttccgcctgacatccgtagatcttgaggactggatggatgttagcctggtgcctgccttcaatgtcctgggtcaggccggctccggcgtcaaacccaagccacaagtctactctaccctcctcaacagtggctgccaagggggcgagcatgcggcctgcttcacagagctgcggaggaactttgtgaacattcgcccagccaagttgaagaacctaatcttgctggtgaagcactggtaccaccaggtgtgcctacaggggttgtggaaggagacgctgcccccggtctatgccctggaattgctgaccatcttcgcctgggagcagggctgtaagaaggatgctttcagcctagccgaaggcctccgaactgtcctgggcctgatccaacagcatcagcacctgtgtgttttctggactgtcaactatggcttcgaggaccctgcagttgggcagttcttgcagcggcagcttaagagacccaggcctgtgatcctggacccagctgaccccacatgggacctggggaatggggcagcctggcactgggatttgctagcccaggaggcagcatcctgctatgaccacccatgctttctgagggggatgggggacccagtgcagtcttggaaggggccg

SEQ ID NO: 138 - RNA, 1029 nt, Homo sapiens
auggacuuguacagcaccccggccgcugcgcuggacagguucguggccagaaggcugcagccgcggaaggaguucguagagaaggcgcggcgcgcucugggcgcccuggccgcugcccugagggagcgcgggggccgccucggugcugcugccccgcgggugcugaaaacugucaagggaggcuccucgggccggggcacagcucucaaggguggcugugauucugaacuugucaucuuccucgacugcuucaagagcuauguggaccagagggcccgccgugcagagauccucagugagaugcgggcaucgcuggaauccugguggcagaacccagucccuggucugagacucacguuuccugagcagagcgugccuggggcccugcaguuccgccugacauccguagaucuugaggacuggauggauguuagccuggugccugccuucaauguccugggucaggccggcuccggcgucaaacccaagccacaagucuacucuacccuccucaacaguggcugccaagggggcgagcaugcggccugcuucacagagcugcggaggaacuuugugaacauucgcccagccaaguugaagaaccuaaucuugcuggugaagcacugguaccaccaggugugccuacagggguuguggaaggagacgcugcccccggucuaugcccuggaauugcugaccaucuucgccugggagcagggcuguaagaaggaugcuuucagccuagccgaaggccuccgaacuguccugggccugauccaacagcaucagcaccuguguguuuucuggacugucaacuauggcuucgaggacccugcaguugggcaguucuugcagcggcagcuuaagagacccaggccugugauccuggacccagcugaccccacaugggaccuggggaauggggcagccuggcacugggauuugcuagcccaggaggcagcauccugcuaugaccacccaugcuuucugagggggaugggggacccagugcagucuuggaaggggccg

SEQ ID NO: 139 - DNA, 1032 bp, Artificial Sequence
  Codon optimised DNA encoding OAS3
atggacctgtacagcacaccagccgccgctctggatagattcgtggctagacgactgcagccccggaaagaattcgtggaaaaggctcggagagccctgggagcacttgctgctgctctgagagaaagaggcggcagacttggagccgctgctcccagagtgctgaaaacagtgaaaggcggcagcagcggcagaggcacagctcttaaaggcggctgcgatagcgagctggtcatcttcctggactgcttcaagagctacgtggaccagagagccagacgggccgagatcctgtctgagatgagagccagcctggaaagctggtggcagaatcctgtgcctggcctgagactgacattccccgaacagtctgttcccggcgctctgcagtttagactgacctccgtggacctggaagattggatggatgtgtccctggtgcctgccttcaatgtgctgggacaagctggctctggcgtgaagcctaagcctcaggtgtactctaccctgctgaactccggctgtcaaggcggagaacacgccgcctgttttaccgagctgcggcggaacttcgtgaacatcagacccgccaagctgaagaacctgatcctgctggtcaagcactggtatcaccaagtgtgcctgcaaggcctgtggaaagaaaccctgcctcctgtgtacgccctggaactgctgaccatcttcgcctgggaacagggctgcaagaaggacgcctttagcctggccgagggcctgagaacagttctgggactgattcagcagcaccagcacctgtgcgtgttctggaccgtgaactacggcttcgaggatcctgccgtgggccagtttctgcagagacagctgaagaggcccagacctgtgatcctggatcctgcagaccctacatgggacctcggaaatggcgctgcctggcattgggatctgctggcccaagaagccgccagctgttacgatcacccctgctttctgagaggcatgggcgatcctgtgcagagctggaagggaccttga

SEQ ID NO: 140 - RNA, 1032 nt, Artificial Sequence
  Codon optimised RNA encoding OAS3
auggaccuguacagcacaccagccgccgcucuggauagauucguggcuagacgacugcagccccggaaagaauucguggaaaaggcucggagagcccugggagcacuugcugcugcucugagagaaagaggcggcagacuuggagccgcugcucccagagugcugaaaacagugaaaggcggcagcagcggcagaggcacagcucuuaaaggcggcugcgauagcgagcuggucaucuuccuggacugcuucaagagcuacguggaccagagagccagacgggccgagauccugucugagaugagagccagccuggaaagcugguggcagaauccugugccuggccugagacugacauuccccgaacagucuguucccggcgcucugcaguuuagacugaccuccguggaccuggaagauuggauggaugugucccuggugccugccuucaaugugcugggacaagcuggcucuggcgugaagccuaagccucagguguacucuacccugcugaacuccggcugucaaggcggagaacacgccgccuguuuuaccgagcugcggcggaacuucgugaacaucagacccgccaagcugaagaaccugauccugcuggucaagcacugguaucaccaagugugccugcaaggccuguggaaagaaacccugccuccuguguacgcccuggaacugcugaccaucuucgccugggaacagggcugcaagaaggacgccuuuagccuggccgagggccugagaacaguucugggacugauucagcagcaccagcaccugugcguguucuggaccgugaacuacggcuucgaggauccugccgugggccaguuucugcagagacagcugaagaggcccagaccugugauccuggauccugcagacccuacaugggaccucggaaauggcgcugccuggcauugggaucugcuggcccaagaagccgccagcuguuacgaucaccccugcuuucugagaggcaugggcgauccugugcagagcuggaagggaccuuga

SEQ ID NO: 141 - protein, 133 aa, Homo sapiens
MQEKPKPVPDKENKKLLCRKCKALACYTADVRVIEECHYTVLGDAFKECFVSRPHPKPKQFSSFEKRAKIFCARQNCSHDWGIHVKYKTFEIPVIKIESFVVEDIATGVQTLYSKWKDFHFEKIPFDPAEMSK

SEQ ID NO: 142 - DNA, 399 bp, Homo sapiens
atgcaagaaaaaccaaaacctgtacctgataaggaaaataaaaaactgctctgcagaaagtgcaaagccttggcatgttacacagctgacgtaagagtgatagaggaatgccattacactgtgcttggagatgcttttaaggaatgctttgtgagtagaccacatcccaagccaaagcagttttcaagttttgaaaaaagagcaaagatattctgtgcccgacagaactgcagccatgactggggaatccatgtgaagtacaagacatttgagattccagttataaaaattgaaagttttgtggtggaggatattgcaactggagttcagacactgtactcgaagtggaaggactttcattttgagaagataccatttgatccagcagaaatgtccaaa

SEQ ID NO: 143 - RNA, 399 nt, Homo sapiens
augcaagaaaaaccaaaaccuguaccugauaaggaaaauaaaaaacugcucugcagaaagugcaaagccuuggcauguuacacagcugacguaagagugauagaggaaugccauuacacugugcuuggagaugcuuuuaaggaaugcuuugugaguagaccacaucccaagccaaagcaguuuucaaguuuugaaaaaagagcaaagauauucugugcccgacagaacugcagccaugacuggggaauccaugugaaguacaagacauuugagauuccaguuauaaaaauugaaaguuuugugguggaggauauugcaacuggaguucagacacuguacucgaaguggaaggacuuucauuuugagaagauaccauuugauccagcagaaauguccaaa

SEQ ID NO: 144 - DNA, 402 bp, Artificial Sequence
  Codon optimised DNA encoding RIG-1
atgcaagagaagcccaagcctgtgcctgacaaagagaacaagaaactgctgtgccggaagtgcaaggccctggcctgttatacagccgacgtgcgcgtgatcgaggaatgccactatacagtgctgggcgacgccttcaaagaatgcttcgtgtcccggcctcatcctaagcctaagcagttcagcagcttcgagaagcgggccaagatcttctgcgccagacagaactgcagccacgactggggaatccacgtgaagtacaagaccttcgagatccccgtgatcaagatcgagagcttcgtggtggaagatatcgccaccggcgtgcagaccctgtacagcaagtggaaggatttccactttgagaagatccctttcgaccccgccgagatgagcaagtga

SEQ ID NO: 145 - RNA, 402 nt, Artificial Sequence
  Codon optimised RNA encoding RIG-1
augcaagagaagcccaagccugugccugacaaagagaacaagaaacugcugugccggaagugcaaggcccuggccuguuauacagccgacgugcgcgugaucgaggaaugccacuauacagugcugggcgacgccuucaaagaaugcuucgugucccggccucauccuaagccuaagcaguucagcagcuucgagaagcgggccaagaucuucugcgccagacagaacugcagccacgacuggggaauccacgugaaguacaagaccuucgagauccccgugaucaagaucgagagcuucgugguggaagauaucgccaccggcgugcagacccuguacagcaaguggaaggauuuccacuuugagaagaucccuuucgaccccgccgagaugagcaaguga

SEQ ID NO: 146 - protein, 650 aa, Homo sapiens
MASNMDREMILADFQACTGIENIDEAITLLEQNNWDLVAAINGVIPQENGILQSEYGGETIPGPAFNPASHPASAPTSSSSSAFRPVMPSRQIVERQPRMLDFRVEYRDRNVDVVLEDTCTVGEIKQILENELQIPVSKMLLKGWKTGDVEDSTVLKSLHLPKNNSLYVLTPDLPPPSSSSHAGALQESLNQNFMLIITHREVQREYNLNFSGSSTIQEVKRNVYDLTSIPVRHQLWEGWPTSATDDSMCLAESGLSYPCHRLTVGRRSSPAQTREQSEEQITDVHMVSDSDGDDFEDATEFGVDDGEVFGMASSALRKSPMMPENAENEGDALLQFTAEFSSRYGDCHPVFFIGSLEAAFQEAFYVKARDRKLLAIYLHHDESVLTNVFCSQMLCAESIVSYLSQNFITWAWDLTKDSNRARFLTMCNRHFGSVVAQTIRTQKTDQFPLFLIIMGKRSSNEVLNVIQGNTTVDELMMRLMAAMEIFTAQQQEDIKDEDEREARENVKREQDEAYRLSLEADRAKREAHEREMAEQFRLEQIRKEQEEEREAIRLSLEQALPPEPKEENAEPVSKLRIRTPSGEFLERRFLASNKLQIVFDFVASKGFPWDEYKLLSTFPRRDVTQLDPNKSLLEVKLFPQETLFLEAKE

SEQ ID NO: 147 - DNA, 1950 bp, Homo sapiens
atggcgtccaacatggaccgggagatgatcctggcggattttcaggcatgtactggcattgaaaacattgacgaagctattacattgcttgaacaaaataattgggacttagtggcagctatcaatggtgtaataccacaggaaaatggcattctacaaagtgaatatggaggtgagaccataccaggacctgcatttaatccagcaagtcatccagcttcagctcctacttcctcttcttcttcagcgtttcgacctgtaatgccatccaggcagattgtagaaaggcaacctcggatgctggacttcagggttgaatacagagacagaaatgttgatgtggtacttgaagacacctgtactgttggagagattaaacagattctagaaaatgaacttcagatacctgtgtccaaaatgctgttaaaaggctggaagacgggagatgtggaagacagtacggtcctaaaatctctacacttgccaaaaaacaacagtctttatgtccttacaccagatttgccaccaccttcatcatctagtcatgctggtgccctgcaggagtcattaaatcaaaacttcatgctgatcatcacccaccgagaagtccagcgggagtacaacctgaacttctcaggaagcagtactattcaagaggtaaagagaaatgtgtatgaccttacaagtatccccgttcgccaccaattatgggagggctggccaacttctgctacagacgactcaatgtgtcttgctgaatcagggctctcttatccctgccatcgacttacagtgggaagaagatcttcacctgcacagacccgggaacagtcggaagaacaaatcaccgatgttcatatggttagtgatagcgatggagatgactttgaagatgctacagaatttggggtggatgatggagaagtatttggcatggcgtcatctgccttgagaaaatctccaatgatgccagaaaacgcagaaaatgaaggagatgccttattacaatttacagcagagttttcttcaagatatggtgattgccatcctgtattttttattggctcattagaagctgcttttcaagaggccttctatgtgaaagcccgagatagaaagcttcttgctatctacctccaccatgatgaaagtgtgttaaccaacgtgttctgctcacaaatgctttgtgctgaatccattgtttcttatctgagtcaaaattttataacctgggcttgggatctgacaaaggactccaacagagcaagatttctcactatgtgcaatagacactttggcagtgttgtggcacaaaccattcggactcaaaaaacggatcagtttccgcttttcctgattattatgggaaagcgatcatctaatgaagtgttgaatgtgatacaagggaacacaacagtagatgagttaatgatgagactcatggctgcaatggagatcttcacagcccaacaacaggaagatataaaggacgaggatgaacgtgaagccagagaaaatgtgaagagagagcaagatgaggcctatcgcctttcacttgaggctgacagagcaaagagggaagctcacgagagagagatggcagaacagtttcgtttggagcagattcgcaaagaacaagaagaggaacgtgaggccatccggctgtccttagagcaagccctgcctcctgagccaaaggaagaaaatgctgagcctgtgagcaaactgcggatccggacccccagtggcgagttcttggagcggcgtttcctggccagcaacaagctccagattgtctttgattttgtagcttccaaaggatttccatgggatgagtacaagttactgagcacctttcctaggagagacgtaactcaactggacccaaataaatcattattggaggtaaagttgttccctcaagaaacccttttccttgaagcaaaagag

SEQ ID NO: 148 - RNA, 1950 nt, Homo sapiens
auggcguccaacauggaccgggagaugauccuggcggauuuucaggcauguacuggcauugaaaacauugacgaagcuauuacauugcuugaacaaaauaauugggacuuaguggcagcuaucaaugguguaauaccacaggaaaauggcauucuacaaagugaauauggaggugagaccauaccaggaccugcauuuaauccagcaagucauccagcuucagcuccuacuuccucuucuucuucagcguuucgaccuguaaugccauccaggcagauuguagaaaggcaaccucggaugcuggacuucaggguugaauacagagacagaaauguugaugugguacuugaagacaccuguacuguuggagagauuaaacagauucuagaaaaugaacuucagauaccuguguccaaaaugcuguuaaaaggcuggaagacgggagauguggaagacaguacgguccuaaaaucucuacacuugccaaaaaacaacagucuuuauguccuuacaccagauuugccaccaccuucaucaucuagucaugcuggugcccugcaggagucauuaaaucaaaacuucaugcugaucaucacccaccgagaaguccagcgggaguacaaccugaacuucucaggaagcaguacuauucaagagguaaagagaaauguguaugaccuuacaaguauccccguucgccaccaauuaugggagggcuggccaacuucugcuacagacgacucaaugugucuugcugaaucagggcucucuuaucccugccaucgacuuacagugggaagaagaucuucaccugcacagacccgggaacagucggaagaacaaaucaccgauguucauaugguuagugauagcgauggagaugacuuugaagaugcuacagaauuugggguggaugauggagaaguauuuggcauggcgucaucugccuugagaaaaucuccaaugaugccagaaaacgcagaaaaugaaggagaugccuuauuacaauuuacagcagaguuuucuucaagauauggugauugccauccuguauuuuuuauuggcucauuagaagcugcuuuucaagaggccuucuaugugaaagcccgagauagaaagcuucuugcuaucuaccuccaccaugaugaaaguguguuaaccaacguguucugcucacaaaugcuuugugcugaauccauuguuucuuaucugagucaaaauuuuauaaccugggcuugggaucugacaaaggacuccaacagagcaagauuucucacuaugugcaauagacacuuuggcaguguuguggcacaaaccauucggacucaaaaaacggaucaguuuccgcuuuuccugauuauuaugggaaagcgaucaucuaaugaaguguugaaugugauacaagggaacacaacaguagaugaguuaaugaugagacucauggcugcaauggagaucuucacagcccaacaacaggaagauauaaaggacgaggaugaacgugaagccagagaaaaugugaagagagagcaagaugaggccuaucgccuuucacuugaggcugacagagcaaagagggaagcucacgagagagagauggcagaacaguuucguuuggagcagauucgcaaagaacaagaagaggaacgugaggccauccggcuguccuuagagcaagcccugccuccugagccaaaggaagaaaaugcugagccugugagcaaacugcggauccggacccccaguggcgaguucuuggagcggcguuuccuggccagcaacaagcuccagauugucuuugauuuuguagcuuccaaaggauuuccaugggaugaguacaaguuacugagcaccuuuccuaggagagacguaacucaacuggacccaaauaaaucauuauuggagguaaaguuguucccucaagaaacccuuuuccuugaagcaaaagag

SEQ ID NO: 149 - DNA, 1953 bp, Artificial Sequence
  Codon optimised DNA encoding FAF1
atggccagcaacatggacagagagatgatcctggccgacttccaggcctgtaccggcatcgagaacatcgacgaggccatcacactgctggaacagaacaactgggatctcgtggccgccatcaacggcgtgatccctcaagagaatggcatcctgcagagcgagtacggcggcgagacaattcctggacctgccttcaatcccgccagccatcctgcatctgcccctacatctagcagcagcagcgccttcagacccgtgatgcctagcagacagatcgtggaacggcagcccagaatgctggacttcagagtcgagtaccgggacagaaacgtggacgtggtgctggaagatacctgcaccgtgggcgagatcaagcagatcctggaaaacgagctgcagatccccgtgtccaagatgctgctgaaaggctggaaaaccggcgacgtggaagatagcaccgtgctgaagtccctgcatctccctaagaacaacagcctgtacgtgctgacccctgacctgcctcctccaagctctagttctcatgctggcgccctgcaagagagcctgaaccagaacttcatgctgatcatcacccaccgcgaggtgcagagagagtataacctgaacttcagcggcagcagcaccatccaagaagtgaagcggaacgtctacgacctgaccagcattcctgtgcggcaccagctttgggaaggctggcctacaagcgccaccgacgattctatgtgtctggccgagagcggcctgagctacccttgtcacagactgaccgtgggcagaagaagcagccctgctcagacaagagagcagtccgaggaacagatcaccgacgtgcacatggtgtccgatagcgacggcgacgatttcgaggatgccaccgagtttggagtggacgacggcgaggttttcggcatggctagcagcgccctgagaaagtcccctatgatgcccgagaacgccgagaatgaaggcgacgccctgctgcagtttaccgccgagtttagcagcagatacggcgactgtcaccccgtgttcttcatcggatctctggaagccgccttccaagaggccttttacgtgaaggccagagacagaaagctgctggctatctatctgcaccacgacgagagcgtgctgacaaacgtgttctgcagccagatgctgtgcgccgagagcatcgtgtcttacctgtctcagaatttcatcacctgggcctgggatctgaccaaggacagcaacagagcccggttcctgaccatgtgtaaccggcactttggcagcgtggtggcccagaccatcagaacccagaaaaccgatcagttccctctgttcctgatcattatgggcaagcgcagcagcaacgaggtgctgaatgtgatccagggcaacaccaccgtggacgagctgatgatgagactgatggccgctatggaaatcttcacagcccagcagcaagaagatatcaaggacgaggacgagcgcgaggcccgcgagaatgtgaaaagagaacaggacgaagcctaccggctgagcctggaagctgacagagccaagagagaggcccacgagagagagatggccgagcagttcagactcgagcagatccgcaaagagcaagaggaagagagagaagccatccggctgtccctggaacaagccttgcctcctgagcctaaagaagagaacgctgagccagtgtccaagctgcggatcagaactcctagcggcgagttcctggaaagacggttcctggcctccaacaaactgcagatcgtgttcgacttcgtggcctctaagggcttcccctgggacgagtacaagctgctgagcacattccccagacgggacgtgacacagctggaccctaacaagagcctgctggaagtgaaactgtttccccaagagacactgtttctcgaggccaaagagtga

SEQ ID NO: 150 - RNA, 1953 nt, Artificial Sequence
  Codon optimised RNA encoding FAF1
auggccagcaacauggacagagagaugauccuggccgacuuccaggccuguaccggcaucgagaacaucgacgaggccaucacacugcuggaacagaacaacugggaucucguggccgccaucaacggcgugaucccucaagagaauggcauccugcagagcgaguacggcggcgagacaauuccuggaccugccuucaaucccgccagccauccugcaucugccccuacaucuagcagcagcagcgccuucagacccgugaugccuagcagacagaucguggaacggcagcccagaaugcuggacuucagagucgaguaccgggacagaaacguggacguggugcuggaagauaccugcaccgugggcgagaucaagcagauccuggaaaacgagcugcagauccccguguccaagaugcugcugaaaggcuggaaaaccggcgacguggaagauagcaccgugcugaagucccugcaucucccuaagaacaacagccuguacgugcugaccccugaccugccuccuccaagcucuaguucucaugcuggcgcccugcaagagagccugaaccagaacuucaugcugaucaucacccaccgcgaggugcagagagaguauaaccugaacuucagcggcagcagcaccauccaagaagugaagcggaacgucuacgaccugaccagcauuccugugcggcaccagcuuugggaaggcuggccuacaagcgccaccgacgauucuaugugucuggccgagagcggccugagcuacccuugucacagacugaccgugggcagaagaagcagcccugcucagacaagagagcaguccgaggaacagaucaccgacgugcacaugguguccgauagcgacggcgacgauuucgaggaugccaccgaguuuggaguggacgacggcgagguuuucggcauggcuagcagcgcccugagaaaguccccuaugaugcccgagaacgccgagaaugaaggcgacgcccugcugcaguuuaccgccgaguuuagcagcagauacggcgacugucaccccguguucuucaucggaucucuggaagccgccuuccaagaggccuuuuacgugaaggccagagacagaaagcugcuggcuaucuaucugcaccacgacgagagcgugcugacaaacguguucugcagccagaugcugugcgccgagagcaucgugucuuaccugucucagaauuucaucaccugggccugggaucugaccaaggacagcaacagagcccgguuccugaccauguguaaccggcacuuuggcagcgugguggcccagaccaucagaacccagaaaaccgaucaguucccucuguuccugaucauuaugggcaagcgcagcagcaacgaggugcugaaugugauccagggcaacaccaccguggacgagcugaugaugagacugauggccgcuauggaaaucuucacagcccagcagcaagaagauaucaaggacgaggacgagcgcgaggcccgcgagaaugugaaaagagaacaggacgaagccuaccggcugagccuggaagcugacagagccaagagagaggcccacgagagagagauggccgagcaguucagacucgagcagauccgcaaagagcaagaggaagagagagaagccauccggcugucccuggaacaagccuugccuccugagccuaaagaagagaacgcugagccaguguccaagcugcggaucagaacuccuagcggcgaguuccuggaaagacgguuccuggccuccaacaaacugcagaucguguucgacuucguggccucuaagggcuuccccugggacgaguacaagcugcugagcacauuccccagacgggacgugacacagcuggacccuaacaagagccugcuggaagugaaacuguuuccccaagagacacuguuucucgaggccaaagaguga

SEQ ID NO: 151 - protein, 211 aa, Homo sapiens
MVAHNQVAADNAVSTAAEPRRRPEPSSSSSSSPAAPARPRPCPAVPAPAPGDTHFRTFRSHADYRRITRASALLDACGFYWGPLSVHGAHERLRAEPVGTFLVRDSRQRNCFFALSVKMASGPTSIRVHFQAGRFHLDGSRESFDCLFELLEHYVAAPRRMLGAPLRQRRVRPLQELCRQRIVATVGRENLARIPLNPVLRDYLSSFPFQI

SEQ ID NO: 152 - DNA, 633 bp, Homo sapiens
atggtagcacacaaccaggtggcagccgacaatgcagtctccacagcagcagagccccgacggcggccagaaccttcctcctcttcctcctcctcgcccgcggcccccgcgcgcccgcggccgtgccccgcggtcccggccccggcccccggcgacacgcacttccgcacattccgttcgcacgccgattaccggcgcatcacgcgcgccagcgcgctcctggacgcctgcggattctactgggggcccctgagcgtgcacggggcgcacgagcggctgcgcgccgagcccgtgggcaccttcctggtgcgcgacagccgccagcggaactgctttttcgcccttagcgtgaagatggcctcgggacccacgagcatccgcgtgcactttcaggccggccgctttcacctggatggcagccgcgagagcttcgactgcctcttcgagctgctggagcactacgtggcggcgccgcgccgcatgctgggggccccgctgcgccagcgccgcgtgcggccgctgcaggagctgtgccgccagcgcatcgtggccaccgtgggccgcgagaacctggctcgcatccccctcaaccccgtcctccgcgactacctgagctccttccccttccagatt

SEQ ID NO: 153 - RNA, 633 nt, Homo sapiens
augguagcacacaaccagguggcagccgacaaugcagucuccacagcagcagagccccgacggcggccagaaccuuccuccucuuccuccuccucgcccgcggcccccgcgcgcccgcggccgugccccgcggucccggccccggcccccggcgacacgcacuuccgcacauuccguucgcacgccgauuaccggcgcaucacgcgcgccagcgcgcuccuggacgccugcggauucuacugggggccccugagcgugcacggggcgcacgagcggcugcgcgccgagcccgugggcaccuuccuggugcgcgacagccgccagcggaacugcuuuuucgcccuuagcgugaagauggccucgggacccacgagcauccgcgugcacuuucaggccggccgcuuucaccuggauggcagccgcgagagcuucgacugccucuucgagcugcuggagcacuacguggcggcgccgcgccgcaugcugggggccccgcugcgccagcgccgcgugcggccgcugcaggagcugugccgccagcgcaucguggccaccgugggccgcgagaaccuggcucgcaucccccucaaccccguccuccgcgacuaccugagcuccuuccccuuccagauu

SEQ ID NO: 154 - DNA, 636 bp, Artificial Sequence
  Codon optimised DNA encoding SOCS1
atggtggcccataatcaggtggccgccgataacgccgtgtctacagctgccgaacctagaagaaggcccgagcctagcagcagcagctctagttctcctgccgctcctgccagacctagaccttgtcctgctgttcctgctccagctcctggcgacacccacttcagaacctttagaagccacgccgactaccggcggatcacaagagcatctgctctgctggatgcctgcggcttttattggggccctctgtctgtgcacggcgcccacgaaagactgagagctgaacctgtgggcaccttcctcgtgcgggatagcagacagcggaactgcttctttgccctgagcgtgaagatggccagcggacccacatccatcagagtgcactttcaggccggcagattccacctggatggcagcagagagagcttcgactgcctgttcgagctgctggaacactacgtggccgctcctagaaggatgctgggagcacccctgagacagagaagagtgcggcctctgcaagagctgtgccggcagagaatcgtggccacagtgggcagagagaacctggccagaattcctctgaaccccgtgctgagagactacctgagcagcttccccttccaaatctga

SEQ ID NO: 155 - RNA, 636 nt, Artificial Sequence
  Codon optimised RNA encoding SOCS1
augguggcccauaaucagguggccgccgauaacgccgugucuacagcugccgaaccuagaagaaggcccgagccuagcagcagcagcucuaguucuccugccgcuccugccagaccuagaccuuguccugcuguuccugcuccagcuccuggcgacacccacuucagaaccuuuagaagccacgccgacuaccggcggaucacaagagcaucugcucugcuggaugccugcggcuuuuauuggggcccucugucugugcacggcgcccacgaaagacugagagcugaaccugugggcaccuuccucgugcgggauagcagacagcggaacugcuucuuugcccugagcgugaagauggccagcggacccacauccaucagagugcacuuucaggccggcagauuccaccuggauggcagcagagagagcuucgacugccuguucgagcugcuggaacacuacguggccgcuccuagaaggaugcugggagcaccccugagacagagaagagugcggccucugcaagagcugugccggcagagaaucguggccacagugggcagagagaaccuggccagaauuccucugaaccccgugcugagagacuaccugagcagcuuccccuuccaaaucuga

SEQ ID NO: 156 - protein, 225 aa, Homo sapiens
MVTHSKFPAAGMSRPLDTSLRLKTFSSKSEYQLVVNAVRKLQESGFYWSAVTGGEANLLLSAEPAGTFLIRDSSDQRHFFTLSVKTQSGTKNLRIQCEGGSFSLQSDPRSTQPVPRFDCVLKLVHHYMPPPGAPSFPSPPTEPSSEVPEQPSAQPLPGSPPRRAYYIYSGGEKIPLVLSRPLSSNVATLQHLCRKTVNGHLDSYEKVTQLPGPIREFLDQYDAPL

SEQ ID NO: 157 - DNA, 675 bp, Homo sapiens
atggtcacccacagcaagtttcccgccgccgggatgagccgccccctggacaccagcctgcgcctcaagaccttcagctccaagagcgagtaccagctggtggtgaacgcagtgcgcaagctgcaggagagcggcttctactggagcgcagtgaccggcggcgaggcgaacctgctgctcagtgccgagcccgccggcacctttctgatccgcgacagctcggaccagcgccacttcttcacgctcagcgtcaagacccagtctgggaccaagaacctgcgcatccagtgtgaggggggcagcttctctctgcagagcgatccccggagcacgcagcccgtgccccgcttcgactgcgtgctcaagctggtgcaccactacatgccgccccctggagccccctccttcccctcgccacctactgaaccctcctccgaggtgcccgagcagccgtctgcccagccactccctgggagtccccccagaagagcctattacatctactccgggggcgagaagatccccctggtgttgagccggcccctctcctccaacgtggccactcttcagcatctctgtcggaagaccgtcaacggccacctggactcctatgagaaagtcacccagctgccggggcccattcgggagttcctggaccagtacgatgccccgctt

SEQ ID NO: 158 - RNA, 675 nt, Homo sapiens
auggucacccacagcaaguuucccgccgccgggaugagccgcccccuggacaccagccugcgccucaagaccuucagcuccaagagcgaguaccagcugguggugaacgcagugcgcaagcugcaggagagcggcuucuacuggagcgcagugaccggcggcgaggcgaaccugcugcucagugccgagcccgccggcaccuuucugauccgcgacagcucggaccagcgccacuucuucacgcucagcgucaagacccagucugggaccaagaaccugcgcauccagugugaggggggcagcuucucucugcagagcgauccccggagcacgcagcccgugccccgcuucgacugcgugcucaagcuggugcaccacuacaugccgcccccuggagcccccuccuuccccucgccaccuacugaacccuccuccgaggugcccgagcagccgucugcccagccacucccugggaguccccccagaagagccuauuacaucuacuccgggggcgagaagaucccccugguguugagccggccccucuccuccaacguggccacucuucagcaucucugucggaagaccgucaacggccaccuggacuccuaugagaaagucacccagcugccggggcccauucgggaguuccuggaccaguacgaugccccgcuu

SEQ ID NO: 159 - DNA, 678 bp, Artificial Sequence
  Codon optimised DNA encoding SOCS3
atggtcacccacagcaagtttccagccgccggaatgagcagacccctggatacaagcctgcggctgaaaaccttcagcagcaagagcgagtatcagctggtggtcaacgccgtgcggaagctgcaagagagcggcttttattggagcgccgtgacaggcggagaggccaatcttctgctgtctgccgaacctgccggcaccttcctgatcagagatagcagcgaccagcggcacttcttcaccctgagcgtgaaaacccagagcggcaccaagaacctgcggatccaatgtgaaggcggcagcttcagcctgcagagcgaccctagatctacccagcctgtgcctagattcgactgcgtgctgaagctcgtgcaccactacatgcctccacctggcgctcctagcttcccatctcctccaacagagcctagcagcgaggtgccagaacagccttctgctcaacctctgcctggcagccctcctagaagggcctactacatctattctggcggcgagaagatccctctggtgctgtctagacccctgagcagcaatgtggccactctgcagcacctgtgcagaaagaccgtgaacggccacctggacagctacgagaaagtgacccaactgcctggacctatcagagagttcctggaccagtacgacgcccctctttga

SEQ ID NO: 160 - RNA, 678 nt, Artificial Sequence
  Codon optimised RNA encoding SOCS3
auggucacccacagcaaguuuccagccgccggaaugagcagaccccuggauacaagccugcggcugaaaaccuucagcagcaagagcgaguaucagcugguggucaacgccgugcggaagcugcaagagagcggcuuuuauuggagcgccgugacaggcggagaggccaaucuucugcugucugccgaaccugccggcaccuuccugaucagagauagcagcgaccagcggcacuucuucacccugagcgugaaaacccagagcggcaccaagaaccugcggauccaaugugaaggcggcagcuucagccugcagagcgacccuagaucuacccagccugugccuagauucgacugcgugcugaagcucgugcaccacuacaugccuccaccuggcgcuccuagcuucccaucuccuccaacagagccuagcagcgaggugccagaacagccuucugcucaaccucugccuggcagcccuccuagaagggccuacuacaucuauucuggcggcgagaagaucccucuggugcugucuagaccccugagcagcaauguggccacucugcagcaccugugcagaaagaccgugaacggccaccuggacagcuacgagaaagugacccaacugccuggaccuaucagagaguuccuggaccaguacgacgccccucuuuga

SEQ ID NO: 161 - protein, 371 aa, Homo sapiens
MSKAFGLLRQICQSILAESSQSPADLEEKKEEDSNMKREQPRERPRAWDYPHGLVGLHNIGQTCCLNSLIQVFVMNVDFTRILKRITVPGADEQRRSVPFQMLLLLEKMQDSRQKAVRPLELAYCLQKCNVPLFVQHDAAQLYLKLWNLIKDQITDVHLVERLQALYTIRVKDSLICVDCAMESSRNSSMLTLPLSLFDVDSKPLKTLEDALHCFFQPRELSSKSKCFCENCGKKTRGKQVLKLTHLPQTLTIHLMRFSIRNSQTRKICHSLYFPQSLDFSQILPMKRESCDAEEQSGGQYELFAVIAHVGMADSGHYCVYIRNAVDGKWFCFNDSNICLVSWEDIQCTYGNPNYHWQETAYLLVYMKMEC

SEQ ID NO: 162 - DNA, 1116 bp, Homo sapiens
atgagcaaggcgtttgggctcctgaggcaaatctgtcagtccatcctggctgagtcctcgcagtccccggcagatcttgaagaaaagaaggaagaagacagcaacatgaagagagagcagcccagagagcgtcccagggcctgggactaccctcatggcctggttggtttacacaacattggacagacctgctgccttaactccttgattcaggtgttcgtaatgaatgtggacttcaccaggatattgaagaggatcacggtgcccaggggagctgacgagcagaggagaagcgtccctttccagatgcttctgctgctggagaagatgcaggacagccggcagaaagcagtgcggcccctggagctggcctactgcctgcagaagtgcaacgtgcccttgtttgtccaacatgatgctgcccaactgtacctcaaactctggaacctgattaaggaccagatcactgatgtgcacttggtggagagactgcaggccctgtatacgatccgggtgaaggactccttgatttgcgttgactgtgccatggagagtagcagaaacagcagcatgctcaccctcccactttctctttttgatgtggactcaaagcccctgaagacactggaggacgccctgcactgcttcttccagcccagggagttatcaagcaaaagcaagtgcttctgtgagaactgtgggaagaagacccgtgggaaacaggtcttgaagctgacccatttgccccagaccctgacaatccacctcatgcgattctccatcaggaattcacagacgagaaagatctgccactccctgtacttcccccagagcttggatttcagccagatccttccaatgaagcgagagtcttgtgatgctgaggagcagtctggagggcagtatgagctttttgctgtgattgcgcacgtgggaatggcagactccggtcattactgtgtctacatccggaatgctgtggatggaaaatggttctgcttcaatgactccaatatttgcttggtgtcctgggaagacatccagtgtacctacggaaatcctaactaccactggcaggaaactgcatatcttctggtttacatgaagatggagtgc

SEQ ID NO: 163 - RNA, 1116 nt, Homo sapiens
augagcaaggcguuugggcuccugaggcaaaucugucaguccauccuggcugaguccucgcaguccccggcagaucuugaagaaaagaaggaagaagacagcaacaugaagagagagcagcccagagagcgucccagggccugggacuacccucauggccugguugguuuacacaacauuggacagaccugcugccuuaacuccuugauucagguguucguaaugaauguggacuucaccaggauauugaagaggaucacggugcccaggggagcugacgagcagaggagaagcgucccuuuccagaugcuucugcugcuggagaagaugcaggacagccggcagaaagcagugcggccccuggagcuggccuacugccugcagaagugcaacgugcccuuguuuguccaacaugaugcugcccaacuguaccucaaacucuggaaccugauuaaggaccagaucacugaugugcacuugguggagagacugcaggcccuguauacgauccgggugaaggacuccuugauuugcguugacugugccauggagaguagcagaaacagcagcaugcucacccucccacuuucucuuuuugauguggacucaaagccccugaagacacuggaggacgcccugcacugcuucuuccagcccagggaguuaucaagcaaaagcaagugcuucugugagaacugugggaagaagacccgugggaaacaggucuugaagcugacccauuugccccagacccugacaauccaccucaugcgauucuccaucaggaauucacagacgagaaagaucugccacucccuguacuucccccagagcuuggauuucagccagauccuuccaaugaagcgagagucuugugaugcugaggagcagucuggagggcaguaugagcuuuuugcugugauugcgcacgugggaauggcagacuccggucauuacugugucuacauccggaaugcuguggauggaaaaugguucugcuucaaugacuccaauauuugcuugguguccugggaagacauccaguguaccuacggaaauccuaacuaccacuggcaggaaacugcauaucuucugguuuacaugaagauggagugc

SEQ ID NO: 164 - DNA, 1119 bp, Artificial Sequence
  Codon optimised DNA encoding USP18
atgagcaaggccttcggcctgctgagacagatctgccagtctatcctggccgagagcagccagtctcctgccgatctggaagagaagaaagaagaggactccaacatgaagcgcgagcagcccagagaaagacccagagcctgggattatcctcacggcctcgtgggcctgcacaatatcggccagacctgctgcctgaacagcctgatccaggtgttcgtgatgaacgtggacttcacccggatcctgaagcggatcacagtgcctagaggcgccgacgagcagagaagatccgtgccttttcagatgctgctgctcctggaaaagatgcaggacagccggcagaaggccgtcagacctctggaactggcctactgcctgcagaaatgcaacgtgcccctgttcgtgcagcacgatgccgctcagctgtacctgaagctgtggaacctgatcaaggaccagatcaccgacgtgcacctggtggaaagactgcaggccctgtacaccatcagagtgaaggactccctgatctgcgtggactgcgccatggaaagcagccggaatagctccatgctgaccctgcctctgagcctgttcgacgtggacagcaagcccctgaaaaccctggaagatgccctgcactgcttcttccagcctagagagctgagcagcaagagcaagtgcttctgcgagaactgcggcaagaaaacccggggcaaacaggtgctgaagctgacccatctgcctcagacactgaccatccacctgatgcggttcagcatccggaacagccagaccagaaagatctgtcactccctgtacttccctcagtctctggacttcagccagattctgcccatgaagagagagagctgcgacgccgaagaacagtctggcggacagtacgagctgttcgccgtgattgcccacgttggcatggccgatagcggccactactgcgtgtacatcagaaacgccgtggacggcaagtggttctgtttcaacgacagcaatatctgcctggtgtcctgggaagatatccagtgcacctacggcaaccccaactaccactggcaagagacagcctacctgctggtgtacatgaagatggaatgctga

SEQ ID NO: 165 - RNA, 1119 nt, Artificial Sequence
  Codon optimised RNA encoding USP18
augagcaaggccuucggccugcugagacagaucugccagucuauccuggccgagagcagccagucuccugccgaucuggaagagaagaaagaagaggacuccaacaugaagcgcgagcagcccagagaaagacccagagccugggauuauccucacggccucgugggccugcacaauaucggccagaccugcugccugaacagccugauccagguguucgugaugaacguggacuucacccggauccugaagcggaucacagugccuagaggcgccgacgagcagagaagauccgugccuuuucagaugcugcugcuccuggaaaagaugcaggacagccggcagaaggccgucagaccucuggaacuggccuacugccugcagaaaugcaacgugccccuguucgugcagcacgaugccgcucagcuguaccugaagcuguggaaccugaucaaggaccagaucaccgacgugcaccugguggaaagacugcaggcccuguacaccaucagagugaaggacucccugaucugcguggacugcgccauggaaagcagccggaauagcuccaugcugacccugccucugagccuguucgacguggacagcaagccccugaaaacccuggaagaugcccugcacugcuucuuccagccuagagagcugagcagcaagagcaagugcuucugcgagaacugcggcaagaaaacccggggcaaacaggugcugaagcugacccaucugccucagacacugaccauccaccugaugcgguucagcauccggaacagccagaccagaaagaucugucacucccuguacuucccucagucucuggacuucagccagauucugcccaugaagagagagagcugcgacgccgaagaacagucuggcggacaguacgagcuguucgccgugauugcccacguuggcauggccgauagcggccacuacugcguguacaucagaaacgccguggacggcaagugguucuguuucaacgacagcaauaucugccugguguccugggaagauauccagugcaccuacggcaaccccaacuaccacuggcaagagacagccuaccugcugguguacaugaagauggaaugcuga

SEQ ID NO: 166 - protein, 565 aa, Homo sapiens
MPQASEHRLGRTREPPVNIQPRVGSKLPFAPRARSKERRNPASGPNPMLRPLPPRPGLPDERLKKLELGRGRTSGPRPRGPLRADHGVPLPGSPPPTVALPLPSRTNLARSKSVSSGDLRPMGIALGGHRGTGELGAALSRLALRPEPPTLRRSTSLRRLGGFPGPPTLFSIRTEPPASHGSFHMISARSSEPFYSDDKMAHHTLLLGSGHVGLRNLGNTCFLNAVLQCLSSTRPLRDFCLRRDFRQEVPGGGRAQELTEAFADVIGALWHPDSCEAVNPTRFRAVFQKYVPSFSGYSQQDAQEFLKLLMERLHLEINRRGRRAPPILANGPVPSPPRRGGALLEEPELSDDDRANLMWKRYLEREDSKIVDLFVGQLKSCLKCQACGYRSTTFEVFCDLSLPIPKKGFAGGKVSLRDCFNLFTKEEELESENAPVCDRCRQKTRSTKKLTVQRFPRILVLHLNRFSASRGSIKKSSVGVDFPLQRLSLGDFASDKAGSPVYQLYALCNHSGSVHYGHYTALCRCQTGWHVYNDSRVSPVSENQVASSEGYVLFYQLMQEPPRCL

SEQ ID NO: 167 - DNA, 1695 bp, Homo sapiens
atgccccaggcctctgagcaccgcctgggccgtacccgagagccacctgttaatatccagccccgagtgggatccaagctaccatttgcccccagggcccgcagcaaggagcgcagaaacccagcctctgggccaaaccccatgttacgacctctgcctccccggccaggtctgcctgatgaacggctcaagaaactggagctgggacggggacggacctcaggccctcgtcccagaggcccccttcgagcagatcatggggttcccctgcctggctcaccacccccaacagtggctttgcctctcccatctcggaccaacttagcccgttccaagtctgtgagcagtggggacttgcgtccaatggggattgccttgggagggcaccgtggcaccggagagcttggggctgcactgagccgcttggccctccggcctgagccacccactttgagacgtagcacttctctccgccgcctagggggctttcctggaccccctaccctgttcagcatacggacagagccccctgcttcccatggctccttccacatgatatccgcccggtcctctgagcctttctactctgatgacaagatggctcatcacacactccttctgggctctggtcatgttggccttcgaaacctgggaaacacgtgcttcctgaatgctgtgctgcagtgtctgagcagcactcgacctcttcgggacttctgtctgagaagggacttccggcaagaggtgcctggaggaggccgagcccaagagctcactgaagcctttgcagatgtgattggtgccctctggcaccctgactcctgcgaagctgtgaatcctactcgattccgagctgtcttccagaaatatgttccctccttctctggatacagccagcaggatgcccaagagttcctgaagctcctcatggagcggctacaccttgaaatcaaccgccgaggccgccgggctccaccgatacttgccaatggtccagttccctctccaccccgccgaggaggggctctgctagaagaacctgagttaagtgatgatgaccgagccaacctaatgtggaaacgttacctggagcgagaggacagcaagattgtggacctgtttgtgggccagttgaaaagttgtctcaagtgccaggcctgtgggtatcgctccacgaccttcgaggttttttgtgacctgtccctgcccatccccaagaaaggatttgctgggggcaaggtgtctctgcgggattgtttcaaccttttcactaaggaagaagagctagagtcggagaatgccccagtgtgtgaccgatgtcggcagaaaactcgaagtaccaaaaagttgacagtacaaagattccctcgaatcctcgtgctccatctgaatcgattttctgcctcccgaggctccatcaaaaaaagttcagtaggtgtagactttccactgcagcgactgagcctaggggactttgccagtgacaaagccggaagtcctgtataccagctgtatgccctttgcaaccactcaggcagcgtccactatggccactacacagccctgtgccggtgccagactggttggcatgtctacaatgactctcgtgtctcccctgtcagtgaaaaccaggtggcatccagcgagggctacgtgctgttctaccaactgatgcaggagccaccccggtgcctg

SEQ ID NO: 168 - RNA, 1695 nt, Homo sapiens
augccccaggccucugagcaccgccugggccguacccgagagccaccuguuaauauccagccccgagugggauccaagcuaccauuugcccccagggcccgcagcaaggagcgcagaaacccagccucugggccaaaccccauguuacgaccucugccuccccggccaggucugccugaugaacggcucaagaaacuggagcugggacggggacggaccucaggcccucgucccagaggcccccuucgagcagaucaugggguuccccugccuggcucaccacccccaacaguggcuuugccucucccaucucggaccaacuuagcccguuccaagucugugagcaguggggacuugcguccaauggggauugccuugggagggcaccguggcaccggagagcuuggggcugcacugagccgcuuggcccuccggccugagccacccacuuugagacguagcacuucucuccgccgccuagggggcuuuccuggacccccuacccuguucagcauacggacagagcccccugcuucccauggcuccuuccacaugauauccgcccgguccucugagccuuucuacucugaugacaagauggcucaucacacacuccuucugggcucuggucauguuggccuucgaaaccugggaaacacgugcuuccugaaugcugugcugcagugucugagcagcacucgaccucuucgggacuucugucugagaagggacuuccggcaagaggugccuggaggaggccgagcccaagagcucacugaagccuuugcagaugugauuggugcccucuggcacccugacuccugcgaagcugugaauccuacucgauuccgagcugucuuccagaaauauguucccuccuucucuggauacagccagcaggaugcccaagaguuccugaagcuccucauggagcggcuacaccuugaaaucaaccgccgaggccgccgggcuccaccgauacuugccaaugguccaguucccucuccaccccgccgaggaggggcucugcuagaagaaccugaguuaagugaugaugaccgagccaaccuaauguggaaacguuaccuggagcgagaggacagcaagauuguggaccuguuugugggccaguugaaaaguugucucaagugccaggccuguggguaucgcuccacgaccuucgagguuuuuugugaccugucccugcccauccccaagaaaggauuugcugggggcaaggugucucugcgggauuguuucaaccuuuucacuaaggaagaagagcuagagucggagaaugccccagugugugaccgaugucggcagaaaacucgaaguaccaaaaaguugacaguacaaagauucccucgaauccucgugcuccaucugaaucgauuuucugccucccgaggcuccaucaaaaaaaguucaguagguguagacuuuccacugcagcgacugagccuaggggacuuugccagugacaaagccggaaguccuguauaccagcuguaugcccuuugcaaccacucaggcagcguccacuauggccacuacacagcccugugccggugccagacugguuggcaugucuacaaugacucucgugucuccccugucagugaaaaccagguggcauccagcgagggcuacgugcuguucuaccaacugaugcaggagccaccccggugccug

SEQ ID NO: 169 - DNA, 1698 bp, Artificial Sequence
  Codon optimised DNA encoding USP21
atgcctcaggcctctgagcacagactgggcagaaccagagaacctcctgtgaacatccagcctagagtgggcagcaagctgcccttcgctcctagagccagaagcaaagagcggagaaaccctgccagcggacccaatcctatgctgaggcctttgcctcctagacctggcctgcctgacgagagactgaagaagctggaactcggcagaggcagaacaagcggccctagacctagaggacctctgagagctgatcacggcgttccactgcctggaagccctccacctacagttgctctgccactgcctagcaggaccaacctggccagatctaagagcgtgtccagcggcgatctgcggcctatgggaattgccctcggaggccatagaggaacaggcgaacttggagccgctctgagcagactggccctcagacctgaacctcctacactgagaagaagcaccagcctgagaaggctcggcggctttcctggaccaccaacactgttcagcatccggacagagcctccagccagccacggcagctttcacatgatcagcgccagatccagcgagcccttctacagcgacgacaagatggcccaccacacactgctgctcggctctggacatgtgggcctgagaaacctgggcaatacctgcttcctgaatgccgtgctgcagtgcctgagcagcacaagacccctgagagacttctgcctgcggcgggactttagacaagaagtgcctggcggaggcagagcccaagaactgacagaggctttcgccgatgtgatcggagccctgtggcaccctgattcttgcgaggccgtgaatcccaccagattccgggccgtgttccagaaatacgtgcccagctttagcggctacagccagcaggatgcccaagagttcctgaagctgctgatggaacggctgcacctggaaatcaacagaagaggcagacgggcccctcctatcctggctaatggacctgttcctagtcctcctagaagaggcggcgctctgctggaagaacctgagctgagcgacgacgacagagccaacctgatgtggaagagatacctggaacgcgaggacagcaagatcgtggatctgttcgtgggccagctgaagtcctgcctgaagtgtcaggcctgtggctacaggtccaccaccttcgaggtgttctgcgacctgtctctgcccattcctaagaagggctttgccggcggaaaggtgtccctgagggactgcttcaacctgttcaccaaagaggaagaactcgagagcgagaacgcccctgtgtgcgacagatgccggcagaaaacccggtccaccaagaaactgaccgtgcagcggttccccagaatcctggtgctgcatctgaacagattctccgccagccggggcagcatcaagaaaagctctgtgggcgtcgacttcccactgcagcgactgagcctgggcgatttcgcctctgataaggccggctctcctgtgtaccagctgtacgccctgtgtaaccacagcggctctgtgcactacggccactacaccgctctgtgtagatgccagacaggctggcacgtgtacaacgacagcagagtgtcccctgtgtccgagaatcaggtggccagctctgagggctacgtgctgttctaccagctgatgcaagagcctcctcggtgcctgtga

SEQ ID NO: 170 - RNA, 1698 nt, Artificial Sequence
  Codon optimised RNA encoding USP21
augccucaggccucugagcacagacugggcagaaccagagaaccuccugugaacauccagccuagagugggcagcaagcugcccuucgcuccuagagccagaagcaaagagcggagaaacccugccagcggacccaauccuaugcugaggccuuugccuccuagaccuggccugccugacgagagacugaagaagcuggaacucggcagaggcagaacaagcggcccuagaccuagaggaccucugagagcugaucacggcguuccacugccuggaagcccuccaccuacaguugcucugccacugccuagcaggaccaaccuggccagaucuaagagcguguccagcggcgaucugcggccuaugggaauugcccucggaggccauagaggaacaggcgaacuuggagccgcucugagcagacuggcccucagaccugaaccuccuacacugagaagaagcaccagccugagaaggcucggcggcuuuccuggaccaccaacacuguucagcauccggacagagccuccagccagccacggcagcuuucacaugaucagcgccagauccagcgagcccuucuacagcgacgacaagauggcccaccacacacugcugcucggcucuggacaugugggccugagaaaccugggcaauaccugcuuccugaaugccgugcugcagugccugagcagcacaagaccccugagagacuucugccugcggcgggacuuuagacaagaagugccuggcggaggcagagcccaagaacugacagaggcuuucgccgaugugaucggagcccuguggcacccugauucuugcgaggccgugaaucccaccagauuccgggccguguuccagaaauacgugcccagcuuuagcggcuacagccagcaggaugcccaagaguuccugaagcugcugauggaacggcugcaccuggaaaucaacagaagaggcagacgggccccuccuauccuggcuaauggaccuguuccuaguccuccuagaagaggcggcgcucugcuggaagaaccugagcugagcgacgacgacagagccaaccugauguggaagagauaccuggaacgcgaggacagcaagaucguggaucuguucgugggccagcugaaguccugccugaagugucaggccuguggcuacagguccaccaccuucgagguguucugcgaccugucucugcccauuccuaagaagggcuuugccggcggaaaggugucccugagggacugcuucaaccuguucaccaaagaggaagaacucgagagcgagaacgccccugugugcgacagaugccggcagaaaacccgguccaccaagaaacugaccgugcagcgguuccccagaauccuggugcugcaucugaacagauucuccgccagccggggcagcaucaagaaaagcucugugggcgucgacuucccacugcagcgacugagccugggcgauuucgccucugauaaggccggcucuccuguguaccagcuguacgcccuguguaaccacagcggcucugugcacuacggccacuacaccgcucuguguagaugccagacaggcuggcacguguacaacgacagcagaguguccccuguguccgagaaucagguggccagcucugagggcuacgugcuguucuaccagcugaugcaagagccuccucggugccuguga

SEQ ID NO: 171 - protein, 438 aa, Homo sapiens
MCKDYVYDKDIEQIAKEEQGEALKLQASTSTEVSHQQCSVPGLGEKFPTWETTKPELELLGHNPRRRRITSSFTIGLRGLINLGNTCFMNCIVQALTHTPILRDFFLSDRHRCEMPSPELCLVCEMSSLFRELYSGNPSPHVPYKLLHLVWIHARHLAGYRQQDAHEFLIAALDVLHRHCKGDDVGKAANNPNHCNCIIDQIFTGGLQSDVTCQACHGVSTTIDPCWDISLDLPGSCTSFWPMSPGRESSVNGESHIPGITTLTDCLRRFTRPEHLGSSAKIKCGSCQSYQESTKQLTMNKLPVVACFHFKRFEHSAKQRRKITTYISFPLELDMTPFMASSKESRMNGQLQLPTNSGNNENKYSLFAVVNHQGTLESGHYTSFIRHHKDQWFKCDDAVITKASIKDVLDSEGYLLFYHKQVLEHESEKVKEMNTQAY

SEQ ID NO: 172 - DNA, 1314 bp, Homo sapiens
atgtgtaaggactatgtatatgacaaagacattgagcaaattgccaaagaagagcaaggagaagctttgaaattacaagcctccacctcaacagaggtttctcaccagcagtgttcagtgccaggccttggtgagaaattcccaacctgggaaacaaccaaaccagaattagaactgctggggcacaacccgaggagaagaagaatcacctccagctttacgatcggtttaagaggactcatcaatcttggcaacacgtgctttatgaactgcattgtccaggccctcacccacacgccgatactgagagatttctttctctctgacaggcaccgatgtgagatgccgagtcccgagttgtgtctggtctgtgagatgtcgtcgctgtttcgggagttgtattctggaaacccgtctcctcatgtgccctataagttactgcacctggtgtggatacatgcccgccatttagcagggtacaggcaacaggatgcccacgagttcctcattgcagcgttagatgtcctgcacaggcactgcaaaggtgatgatgtcgggaaggcggccaacaatcccaaccactgtaactgcatcatagaccaaatcttcacaggtggcctgcagtctgatgtcacctgtcaagcctgccatggcgtctccaccacgatagacccatgctgggacattagtttggacttgcctggctcttgcacctccttctggcccatgagcccagggagggagagcagtgtgaacggggaaagccacataccaggaatcaccaccctcacggactgcttgcggaggtttacgaggccagagcacttaggaagcagtgccaaaatcaaatgtggtagttgccaaagctaccaggaatctaccaaacagctcacaatgaataaattacctgtcgttgcctgttttcatttcaaacggtttgaacattcagcgaaacagaggcgcaagatcactacatacatttcctttcctctggagctggatatgacgccgtttatggcctcaagtaaagagagcagaatgaatggacaattgcagctgccaaccaatagtggaaacaacgaaaataagtattccttgtttgctgtggttaatcaccaaggaaccttggagagtggccactataccagcttcatccggcaccacaaggaccagtggttcaagtgtgatgatgccgtcatcactaaggccagtattaaggacgtactggacagtgaagggtatttactgttctatcacaaacaggtgctagaacatgagtcagaaaaagtgaaagaaatgaacacacaagcctac

SEQ ID NO: 173 - RNA, 1314 nt, Homo sapiens
auguguaaggacuauguauaugacaaagacauugagcaaauugccaaagaagagcaaggagaagcuuugaaauuacaagccuccaccucaacagagguuucucaccagcaguguucagugccaggccuuggugagaaauucccaaccugggaaacaaccaaaccagaauuagaacugcuggggcacaacccgaggagaagaagaaucaccuccagcuuuacgaucgguuuaagaggacucaucaaucuuggcaacacgugcuuuaugaacugcauuguccaggcccucacccacacgccgauacugagagauuucuuucucucugacaggcaccgaugugagaugccgagucccgaguugugucuggucugugagaugucgucgcuguuucgggaguuguauucuggaaacccgucuccucaugugcccuauaaguuacugcaccugguguggauacaugcccgccauuuagcaggguacaggcaacaggaugcccacgaguuccucauugcagcguuagauguccugcacaggcacugcaaaggugaugaugucgggaaggcggccaacaaucccaaccacuguaacugcaucauagaccaaaucuucacagguggccugcagucugaugucaccugucaagccugccauggcgucuccaccacgauagacccaugcugggacauuaguuuggacuugccuggcucuugcaccuccuucuggcccaugagcccagggagggagagcagugugaacggggaaagccacauaccaggaaucaccacccucacggacugcuugcggagguuuacgaggccagagcacuuaggaagcagugccaaaaucaaaugugguaguugccaaagcuaccaggaaucuaccaaacagcucacaaugaauaaauuaccugucguugccuguuuucauuucaaacgguuugaacauucagcgaaacagaggcgcaagaucacuacauacauuuccuuuccucuggagcuggauaugacgccguuuauggccucaaguaaagagagcagaaugaauggacaauugcagcugccaaccaauaguggaaacaacgaaaauaaguauuccuuguuugcugugguuaaucaccaaggaaccuuggagaguggccacuauaccagcuucauccggcaccacaaggaccagugguucaagugugaugaugccgucaucacuaaggccaguauuaaggacguacuggacagugaaggguauuuacuguucuaucacaaacaggugcuagaacaugagucagaaaaagugaaagaaaugaacacacaagccuac

SEQ ID NO: 174 - DNA, 1317 bp, Artificial Sequence
  Codon optimised DNA encoding USP27
atgtgcaaggactacgtgtacgacaaggacatcgagcagatcgccaaagaggaacagggcgaagccctgaagctgcaggccagcacatctacagaggtgtcccaccagcagtgtagcgtgccaggactgggcgagaagttccctacctgggaaaccaccaagcctgagctggaactgctgggccacaatcctcggcggagaagaatcaccagcagcttcaccatcggcctgcggggcctgatcaatctgggcaatacctgcttcatgaactgcatcgtgcaggccctgacacacacccctatcctgagagacttcttcctgtccgaccggcacagatgcgagatgccttctccagagctgtgcctcgtgtgcgagatgagcagcctgttccgggaactgtacagcggcaacccttctcctcacgtgccctacaaactgctgcacctcgtgtggattcacgccagacacctggccggctacagacagcaggatgcccacgagtttctgatcgccgctctggacgtgctgcacagacactgcaaaggcgacgatgtgggcaaagccgccaacaatcccaaccactgcaactgcatcatcgaccagatcttcacaggcggcctgcagagcgacgttacctgtcaagcttgtcacggcgtgtccaccaccatcgatccctgctgggatatcagcctggatctgcctggcagctgcaccagcttttggcctatgagccctggcagagaaagcagcgtgaacggcgagtctcacatccccggcatcaccacactgaccgactgcctgcggagattcaccagacctgagcacctgggaagcagcgccaagatcaagtgtggctcctgccagagctaccaagagagcaccaagcagctgaccatgaacaagctgcctgtggtggcctgcttccacttcaagagattcgagcactccgccaagcagcggcggaagatcacaacctacatcagcttccctctggaactggacatgacccctttcatggccagcagcaaagaaagccggatgaacggccagctccagctgcctacaaatagcggcaacaacgagaacaagtactccctgttcgccgtggtcaaccaccagggcacactggaaagcggccactacaccagcttcatcagacaccacaaggaccagtggttcaagtgcgacgacgccgtgatcaccaaggccagcatcaaggatgtcctggacagcgagggctacctgctgttctaccacaaacaggtgctggaacacgagagcgagaaagtgaaagagatgaacacccaggcctactga

SEQ ID NO: 175 - RNA, 1317 nt, Artificial Sequence
  Codon optimised RNA encoding USP27
augugcaaggacuacguguacgacaaggacaucgagcagaucgccaaagaggaacagggcgaagcccugaagcugcaggccagcacaucuacagaggugucccaccagcaguguagcgugccaggacugggcgagaaguucccuaccugggaaaccaccaagccugagcuggaacugcugggccacaauccucggcggagaagaaucaccagcagcuucaccaucggccugcggggccugaucaaucugggcaauaccugcuucaugaacugcaucgugcaggcccugacacacaccccuauccugagagacuucuuccuguccgaccggcacagaugcgagaugccuucuccagagcugugccucgugugcgagaugagcagccuguuccgggaacuguacagcggcaacccuucuccucacgugcccuacaaacugcugcaccucguguggauucacgccagacaccuggccggcuacagacagcaggaugcccacgaguuucugaucgccgcucuggacgugcugcacagacacugcaaaggcgacgaugugggcaaagccgccaacaaucccaaccacugcaacugcaucaucgaccagaucuucacaggcggccugcagagcgacguuaccugucaagcuugucacggcguguccaccaccaucgaucccugcugggauaucagccuggaucugccuggcagcugcaccagcuuuuggccuaugagcccuggcagagaaagcagcgugaacggcgagucucacauccccggcaucaccacacugaccgacugccugcggagauucaccagaccugagcaccugggaagcagcgccaagaucaaguguggcuccugccagagcuaccaagagagcaccaagcagcugaccaugaacaagcugccugugguggccugcuuccacuucaagagauucgagcacuccgccaagcagcggcggaagaucacaaccuacaucagcuucccucuggaacuggacaugaccccuuucauggccagcagcaaagaaagccggaugaacggccagcuccagcugccuacaaauagcggcaacaacgagaacaaguacucccuguucgccguggucaaccaccagggcacacuggaaagcggccacuacaccagcuucaucagacaccacaaggaccagugguucaagugcgacgacgccgugaucaccaaggccagcaucaaggauguccuggacagcgagggcuaccugcuguucuaccacaaacaggugcuggaacacgagagcgagaaagugaaagagaugaacacccaggccuacuga

SEQ ID NO: 176 - protein, 956 aa, Homo sapiens
MSSGLWSQEKVTSPYWEERIFYLLLQECSVTDKQTQKLLKVPKGSIGQYIQDRSVGHSRIPSAKGKKNQIGLKILEQPHAVLFVDEKDVVEINEKFTELLLAITNCEERFSLFKNRNRLSKGLQIDVGCPVKVQLRSGEEKFPGVVRFRGPLLAERTVSGIFFGVELLEEGRGQGFTDGVYQGKQLFQCDEDCGVFVALDKLELIEDDDTALESDYAGPGDTMQVELPPLEINSRVSLKVGETIESGTVIFCDVLPGKESLGYFVGVDMDNPIGNWDGRFDGVQLCSFACVESTILLHINDIIPALSESVTQERRPPKLAFMSRGVGDKGSSSHNKPKATGSTSDPGNRNRSELFYTLNGSSVDSQPQSKSKNTWYIDEVAEDPAKSLTEISTDFDRSSPPLQPPPVNSLTTENRFHSLPFSLTKMPNTNGSIGHSPLSLSAQSVMEELNTAPVQESPPLAMPPGNSHGLEVGSLAEVKENPPFYGVIRWIGQPPGLNEVLAGLELEDECAGCTDGTFRGTRYFTCALKKALFVKLKSCRPDSRFASLQPVSNQIERCNSLAFGGYLSEVVEENTPPKMEKEGLEIMIGKKKGIQGHYNSCYLDSTLFCLFAFSSVLDTVLLRPKEKNDVEYYSETQELLRTEIVNPLRIYGYVCATKIMKLRKILEKVEAASGFTSEEKDPEEFLNILFHHILRVEPLLKIRSAGQKVQDCYFYQIFMEKNEKVGVPTIQQLLEWSFINSNLKFAEAPSCLIIQMPRFGKDFKLFKKIFPSLELNITDLLEDTPRQCRICGGLAMYECRECYDDPDISAGKIKQFCKTCNTQVHLHPKRLNHKYNPVSLPKDLPDWDWRHGCIPCQNMELFAVLCIETSHYVAFVKYGKDDSAWLFFDSMADRDGGQNGFNIPQVTPCPEVGEYLKMSLEDLHSLDSRRIQGCARRLLCDAYMCMYQSPTMSLYK

SEQ ID NO: 177 - DNA, 2868 bp, Homo sapiens
atgagttcaggcttatggagccaagaaaaagtcacttcaccctactgggaagagcggattttttacttgcttcttcaagaatgcagcgttacagacaaacaaacacaaaagctccttaaagtaccgaagggaagtataggacagtatattcaagatcgttctgtggggcattcaaggattccttctgcaaaaggcaagaaaaatcagattggattaaaaattctagagcaacctcatgcagttctctttgttgatgaaaaggatgttgtagagataaatgaaaagttcacagagttacttttggcaattaccaattgtgaggagaggttcagcctgtttaaaaacagaaacagactaagtaaaggcctccaaatagacgtgggctgtcctgtgaaagtacagctgagatctggggaagaaaaatttcctggagttgtacgcttcagaggacccctgttagcagagaggacagtctccggaatattctttggagttgaattgctggaagaaggtcgtggtcaaggtttcactgacggggtgtaccaagggaaacagctttttcagtgtgatgaagattgtggcgtgtttgttgcattggacaagctagaactcatagaagatgatgacactgcattggaaagtgattacgcaggtcctggggacacaatgcaggtcgaacttcctcctttggaaataaactccagagtttctttgaaggttggagaaacaatagaatctggaacagttatattctgtgatgttttgccaggaaaagaaagcttaggatattttgttggtgtggacatggataaccctattggcaactgggatggaagatttgatggagtgcagctttgtagttttgcgtgtgttgaaagtacaattctattgcacatcaatgatatcatcccagctttatcagagagtgtgacgcaggaaaggaggcctcccaaacttgcctttatgtcaagaggtgttggggacaaaggttcatccagtcataataaaccaaaggctacaggatctacctcagaccctggaaatagaaacagatctgaattattttataccttaaatgggtcttctgttgactcacaaccacaatccaaatcaaaaaatacatggtacattgatgaagttgcagaagaccctgcaaaatctcttacagagatatctacagactttgaccgttcttcaccaccactccagcctcctcctgtgaactcactgaccaccgagaacagattccactctttaccattcagtctcaccaagatgcccaataccaatggaagtattggccacagtccactttctctgtcagcccagtctgtaatggaagagctaaacactgcacccgtccaagagagtccacccttggccatgcctcctgggaactcacatggtctagaagtgggctcattggctgaagttaaggagaaccctcctttctatggggtaatccgttggatcggtcagccaccaggactgaatgaagtgctcgctggactggaactggaagatgagtgtgcaggctgtacggatggaaccttcagaggcactcggtatttcacctgtgccctgaagaaggcgctgtttgtgaaactgaagagctgcaggcctgactctaggtttgcatcattgcagccggtttccaatcagattgagcgctgtaactctttagcatttggaggctacttaagtgaagtagtagaagaaaatactccaccaaaaatggaaaaagaaggcttggagataatgattgggaagaagaaaggcatccagggtcattacaattcttgttacttagactcaaccttattctgcttatttgcttttagttctgttctggacactgtgttacttagacccaaagaaaagaacgatgtagaatattatagtgaaacccaagagctactgaggacagaaattgttaatcctctgagaatatatggatatgtgtgtgccacaaaaattatgaaactgaggaaaatacttgaaaaggtggaggctgcatcaggatttacctctgaagaaaaagatcctgaggaattcttgaatattctgtttcatcatattttaagggtagaacctttgctaaaaataagatcagcaggtcaaaaggtacaagattgttacttctatcaaatttttatggaaaaaaatgagaaagttggcgttcccacaattcagcagttgttagaatggtcttttatcaacagtaacctgaaatttgcagaggcaccatcatgtctgattattcagatgcctcgatttggaaaagactttaaactatttaaaaaaatttttccttctctggaattaaatataacagatttacttgaagacactcccagacagtgccggatatgtggagggcttgcaatgtatgagtgtagagaatgctacgacgatccggacatctcagctggaaaaatcaagcagttttgtaaaacctgcaacactcaagtccaccttcatccgaagaggctgaatcataaatataacccagtgtcacttcccaaagacttacccgactgggactggagacacggctgcatcccttgccagaatatggagttatttgctgttctctgcatagaaacaagccactatgttgcttttgtgaagtatgggaaggacgattctgcctggctcttctttgacagcatggccgatcgggatggtggtcagaatggcttcaacattcctcaagtcaccccatgcccagaagtaggagagtacttgaagatgtctctggaagacctgcattccttggactccaggagaatccaaggctgtgcacgaagactgctttgtgatgcatatatgtgcatgtaccagagtccaacaatgagtttgtacaaa

SEQ ID NO: 178 - RNA, 2868 nt, Homo sapiens
augaguucaggcuuauggagccaagaaaaagucacuucacccuacugggaagagcggauuuuuuacuugcuucuucaagaaugcagcguuacagacaaacaaacacaaaagcuccuuaaaguaccgaagggaaguauaggacaguauauucaagaucguucuguggggcauucaaggauuccuucugcaaaaggcaagaaaaaucagauuggauuaaaaauucuagagcaaccucaugcaguucucuuuguugaugaaaaggauguuguagagauaaaugaaaaguucacagaguuacuuuuggcaauuaccaauugugaggagagguucagccuguuuaaaaacagaaacagacuaaguaaaggccuccaaauagacgugggcuguccugugaaaguacagcugagaucuggggaagaaaaauuuccuggaguuguacgcuucagaggaccccuguuagcagagaggacagucuccggaauauucuuuggaguugaauugcuggaagaaggucguggucaagguuucacugacgggguguaccaagggaaacagcuuuuucagugugaugaagauuguggcguguuuguugcauuggacaagcuagaacucauagaagaugaugacacugcauuggaaagugauuacgcagguccuggggacacaaugcaggucgaacuuccuccuuuggaaauaaacuccagaguuucuuugaagguuggagaaacaauagaaucuggaacaguuauauucugugauguuuugccaggaaaagaaagcuuaggauauuuuguugguguggacauggauaacccuauuggcaacugggauggaagauuugauggagugcagcuuuguaguuuugcguguguugaaaguacaauucuauugcacaucaaugauaucaucccagcuuuaucagagagugugacgcaggaaaggaggccucccaaacuugccuuuaugucaagagguguuggggacaaagguucauccagucauaauaaaccaaaggcuacaggaucuaccucagacccuggaaauagaaacagaucugaauuauuuuauaccuuaaaugggucuucuguugacucacaaccacaauccaaaucaaaaaauacaugguacauugaugaaguugcagaagacccugcaaaaucucuuacagagauaucuacagacuuugaccguucuucaccaccacuccagccuccuccugugaacucacugaccaccgagaacagauuccacucuuuaccauucagucucaccaagaugcccaauaccaauggaaguauuggccacaguccacuuucucugucagcccagucuguaauggaagagcuaaacacugcacccguccaagagaguccacccuuggccaugccuccugggaacucacauggucuagaagugggcucauuggcugaaguuaaggagaacccuccuuucuaugggguaauccguuggaucggucagccaccaggacugaaugaagugcucgcuggacuggaacuggaagaugagugugcaggcuguacggauggaaccuucagaggcacucgguauuucaccugugcccugaagaaggcgcuguuugugaaacugaagagcugcaggccugacucuagguuugcaucauugcagccgguuuccaaucagauugagcgcuguaacucuuuagcauuuggaggcuacuuaagugaaguaguagaagaaaauacuccaccaaaaauggaaaaagaaggcuuggagauaaugauugggaagaagaaaggcauccagggucauuacaauucuuguuacuuagacucaaccuuauucugcuuauuugcuuuuaguucuguucuggacacuguguuacuuagacccaaagaaaagaacgauguagaauauuauagugaaacccaagagcuacugaggacagaaauuguuaauccucugagaauauauggauaugugugugccacaaaaauuaugaaacugaggaaaauacuugaaaagguggaggcugcaucaggauuuaccucugaagaaaaagauccugaggaauucuugaauauucuguuucaucauauuuuaaggguagaaccuuugcuaaaaauaagaucagcaggucaaaagguacaagauuguuacuucuaucaaauuuuuauggaaaaaaaugagaaaguuggcguucccacaauucagcaguuguuagaauggucuuuuaucaacaguaaccugaaauuugcagaggcaccaucaugucugauuauucagaugccucgauuuggaaaagacuuuaaacuauuuaaaaaaauuuuuccuucucuggaauuaaauauaacagauuuacuugaagacacucccagacagugccggauauguggagggcuugcaauguaugaguguagagaaugcuacgacgauccggacaucucagcuggaaaaaucaagcaguuuuguaaaaccugcaacacucaaguccaccuucauccgaagaggcugaaucauaaauauaacccagugucacuucccaaagacuuacccgacugggacuggagacacggcugcaucccuugccagaauauggaguuauuugcuguucucugcauagaaacaagccacuauguugcuuuugugaaguaugggaaggacgauucugccuggcucuucuuugacagcauggccgaucgggaugguggucagaauggcuucaacauuccucaagucaccccaugcccagaaguaggagaguacuugaagaugucucuggaagaccugcauuccuuggacuccaggagaauccaaggcugugcacgaagacugcuuugugaugcauauaugugcauguaccagaguccaacaaugaguuuguacaaa

SEQ ID NO: 179 - DNA, 2871 bp, Artificial Sequence
  Codon optimised DNA encoding CYLD
atgtctagcggcctgtggtcccaagagaaagtgacaagcccctactgggaagagaggatcttctacctgctgctgcaagagtgcagcgtgaccgacaagcagacccagaaactgctgaaggtgcccaagggcagcatcggccagtacatccaggatagaagcgtgggccacagcagaatccctagcgccaagggcaagaagaaccagatcggcctgaagatcctggaacagcctcacgccgtgctgttcgtggacgagaaggacgtggtggaaatcaacgagaagttcaccgagctgctgctggccatcaccaactgcgaggaacggttcagcctgttcaagaaccggaaccggctgagcaagggcctgcagatcgatgtgggatgccctgtgaaggtgcagctgagaagcggcgaagagaagttccctggcgtcgtgcggtttagaggacctctgctggccgagagaaccgtgtccggcatcttctttggcgtggaactgctggaagaaggcagaggccagggctttaccgatggcgtgtaccagggcaagcagctgtttcagtgcgacgaggattgcggcgtgttcgtggccctggataagctggaactgatcgaggacgacgacacagccctggaaagcgattatgccggacctggcgataccatgcaggtcgaactgcctccactcgagatcaacagccgggtgtccctgaaagtgggcgagacaatcgagagcggcaccgtgatcttttgcgacgtgctgcctggcaaagagtccctgggctattttgtgggcgtcgacatggacaaccccatcggcaattgggacggcagatttgacggcgtgcagctgtgcagcttcgcctgtgtggaaagcaccatcctgctgcacatcaacgacatcatccccgctctgagcgagagcgtgacccaagaaagacggcctcctaagctggccttcatgtctagaggcgtgggcgataagggcagctccagccacaacaagcctaaggccacaggctccacaagcgaccccggcaacagaaacagaagcgagctgttctacaccctgaacggcagcagcgtggacagccagcctcagagcaagagcaagaacacctggtacatcgacgaggtggccgaggatcctgccaagagcctgacagagatcagcaccgacttcgacagaagcagccctccactgcagcctccacctgtgaatagcctgaccaccgagaacagattccacagcctgcctttcagcctgactaagatgcccaacaccaacggctccatcgggcactctccactgtctctgtctgcccagagcgtgatggaagaactgaacacagcccctgtgcaagagtcccctcctctggctatgcctcctggcaattctcacggcctggaagtgggatctctggccgaagtgaaagagaaccctcctttctacggcgtgatccggtggatcggacaacctcctggactgaatgaagtgctggccggactggaactggaagatgagtgtgccggctgcaccgacggcacctttagaggcaccagatacttcacatgcgccctgaagaaagccctgttcgtgaagctgaagtcctgcagacccgacagcagattcgctagcctgcagcctgtgtccaatcagatcgagcggtgcaactccctggcctttggcggctatctgtccgaggtggtggaagagaacacccctcctaagatggaaaaagagggcctcgagattatgatcgggaagaagaagggcatccaggggcactacaatagctgctacctggacagcaccctgttctgcctgttcgcctttagcagcgtgctggacactgtgctgctgcggcccaaagagaagaacgacgtcgagtactacagcgagacacaagagctgctgagaaccgagatcgtgaaccctctgcggatctacggctacgtgtgcgccaccaagatcatgaagctgcggaagattctggaaaaggtggaagccgcctccggcttcaccagcgaggaaaaggatcccgaagagttcctgaacatcctgtttcaccacatcctgagagtggaacccctgctgaagatcagatccgccggacagaaagtgcaggactgctacttctaccagatcttcatggaaaagaacgagaaagtcggcgtgcccaccatccagcaactgctcgagtggtccttcatcaacagcaacctgaagttcgccgaggctcccagctgcctgatcatccagatgcctagattcggcaaggacttcaagctgttcaaaaagatcttccccagcctcgagctgaacatcaccgacctgctcgaggacacccctcggcagtgtagaatttgtggcggcctggctatgtacgagtgcagagagtgctacgacgaccccgatatcagcgccggcaagatcaagcagttctgcaagacctgcaacacccaagtgcatctgcaccccaagcggctgaaccacaagtacaaccccgtgtctctgcccaaggacctgcctgactgggattggagacacggctgtatcccttgccagaacatggaactgttcgctgtgctgtgcatcgagacaagccactacgtggccttcgtgaagtacggcaaggatgacagcgcctggctgttcttcgacagcatggccgatagagatggcggccagaacggcttcaacatccctcaagtgaccccttgtcctgaagtgggagagtacctgaagatgagcctggaagatctgcacagcctggactccagacggatccagggatgtgctagaaggctgctgtgcgacgcctacatgtgcatgtatcagagccccaccatgagcctgtacaagtga

SEQ ID NO: 180 - RNA, 2871 nt, Artificial Sequence
  Codon optimised RNA encoding CYLD
augucuagcggccuguggucccaagagaaagugacaagccccuacugggaagagaggaucuucuaccugcugcugcaagagugcagcgugaccgacaagcagacccagaaacugcugaaggugcccaagggcagcaucggccaguacauccaggauagaagcgugggccacagcagaaucccuagcgccaagggcaagaagaaccagaucggccugaagauccuggaacagccucacgccgugcuguucguggacgagaaggacgugguggaaaucaacgagaaguucaccgagcugcugcuggccaucaccaacugcgaggaacgguucagccuguucaagaaccggaaccggcugagcaagggccugcagaucgaugugggaugcccugugaaggugcagcugagaagcggcgaagagaaguucccuggcgucgugcgguuuagaggaccucugcuggccgagagaaccguguccggcaucuucuuuggcguggaacugcuggaagaaggcagaggccagggcuuuaccgauggcguguaccagggcaagcagcuguuucagugcgacgaggauugcggcguguucguggcccuggauaagcuggaacugaucgaggacgacgacacagcccuggaaagcgauuaugccggaccuggcgauaccaugcaggucgaacugccuccacucgagaucaacagccgggugucccugaaagugggcgagacaaucgagagcggcaccgugaucuuuugcgacgugcugccuggcaaagagucccugggcuauuuugugggcgucgacauggacaaccccaucggcaauugggacggcagauuugacggcgugcagcugugcagcuucgccuguguggaaagcaccauccugcugcacaucaacgacaucauccccgcucugagcgagagcgugacccaagaaagacggccuccuaagcuggccuucaugucuagaggcgugggcgauaagggcagcuccagccacaacaagccuaaggccacaggcuccacaagcgaccccggcaacagaaacagaagcgagcuguucuacacccugaacggcagcagcguggacagccagccucagagcaagagcaagaacaccugguacaucgacgagguggccgaggauccugccaagagccugacagagaucagcaccgacuucgacagaagcagcccuccacugcagccuccaccugugaauagccugaccaccgagaacagauuccacagccugccuuucagccugacuaagaugcccaacaccaacggcuccaucgggcacucuccacugucucugucugcccagagcgugauggaagaacugaacacagccccugugcaagaguccccuccucuggcuaugccuccuggcaauucucacggccuggaagugggaucucuggccgaagugaaagagaacccuccuuucuacggcgugauccgguggaucggacaaccuccuggacugaaugaagugcuggccggacuggaacuggaagaugagugugccggcugcaccgacggcaccuuuagaggcaccagauacuucacaugcgcccugaagaaagcccuguucgugaagcugaaguccugcagacccgacagcagauucgcuagccugcagccuguguccaaucagaucgagcggugcaacucccuggccuuuggcggcuaucuguccgaggugguggaagagaacaccccuccuaagauggaaaaagagggccucgagauuaugaucgggaagaagaagggcauccaggggcacuacaauagcugcuaccuggacagcacccuguucugccuguucgccuuuagcagcgugcuggacacugugcugcugcggcccaaagagaagaacgacgucgaguacuacagcgagacacaagagcugcugagaaccgagaucgugaacccucugcggaucuacggcuacgugugcgccaccaagaucaugaagcugcggaagauucuggaaaagguggaagccgccuccggcuucaccagcgaggaaaaggaucccgaagaguuccugaacauccuguuucaccacauccugagaguggaaccccugcugaagaucagauccgccggacagaaagugcaggacugcuacuucuaccagaucuucauggaaaagaacgagaaagucggcgugcccaccauccagcaacugcucgagugguccuucaucaacagcaaccugaaguucgccgaggcucccagcugccugaucauccagaugccuagauucggcaaggacuucaagcuguucaaaaagaucuuccccagccucgagcugaacaucaccgaccugcucgaggacaccccucggcaguguagaauuuguggcggccuggcuauguacgagugcagagagugcuacgacgaccccgauaucagcgccggcaagaucaagcaguucugcaagaccugcaacacccaagugcaucugcaccccaagcggcugaaccacaaguacaaccccgugucucugcccaaggaccugccugacugggauuggagacacggcuguaucccuugccagaacauggaacuguucgcugugcugugcaucgagacaagccacuacguggccuucgugaaguacggcaaggaugacagcgccuggcuguucuucgacagcauggccgauagagauggcggccagaacggcuucaacaucccucaagugaccccuuguccugaagugggagaguaccugaagaugagccuggaagaucugcacagccuggacuccagacggauccagggaugugcuagaaggcugcugugcgacgccuacaugugcauguaucagagccccaccaugagccuguacaaguga

SEQ ID NO: 181 - protein, 678 aa, Homo sapiens
MELRSYQWEVIMPALEGKNIIIWLPTGAGKTRAAAYVAKRHLETVDGAKVVVLVNRVHLVTQHGEEFRRMLDGRWTVTTLSGDMGPRAGFGHLARCHDLLICTAELLQMALTSPEEEEHVELTVFSLIVVDECHHTHKDTVYNVIMSQYLELKLQRAQPLPQVLGLTASPGTGGASKLDGAINHVLQLCANLDTWCIMSPQNCCPQLQEHSQQPCKQYNLCHRRSQDPFGDLLKKLMDQIHDHLEMPELSRKFGTQMYEQQVVKLSEAAALAGLQEQRVYALHLRRYNDALLIHDTVRAVDALAALQDFYHREHVTKTQILCAERRLLALFDDRKNELAHLATHGPENPKLEMLEKILQRQFSSSNSPRGIIFTRTRQSAHSLLLWLQQQQGLQTVDIRAQLLIGAGNSSQSTHMTQRDQQEVIQKFQDGTLNLLVATSVAEEGLDIPHCNVVVRYGLLTNEISMVQARGRARADQSVYAFVATEGSRELKRELINEALETLMEQAVAAVQKMDQAEYQAKIRDLQQAALTKRAAQAAQRENQRQQFPVEHVQLLCINCMVAVGHGSDLRKVEGTHHVNVNPNFSNYYNVSRDPVVINKVFKDWKPGGVISCRNCGEVWGLQMIYKSVKLPVLKVRSMLLETPQGRIQAKKWSRVPFSVPDFDFLQHCAENLSDLSLD

SEQ ID NO: 182 - DNA, 2034 bp, Homo sapiens
atggagcttcggtcctaccaatgggaggtgatcatgcctgccctggagggcaagaatatcatcatctggctgcccacgggtgccgggaagacccgggcggctgcttatgtggccaagcggcacctagagactgtggatggagccaaggtggttgtattggtcaacagggtgcacctggtgacccagcatggtgaagagttcaggcgcatgctggatggacgctggaccgtgacaaccctgagtggggacatgggaccacgtgctggctttggccacctggcccggtgccatgacctgctcatctgcacagcagagcttctgcagatggcactgaccagccccgaggaggaggagcacgtggagctcactgtcttctccctgatcgtggtggatgagtgccaccacacgcacaaggacaccgtctacaacgtcatcatgagccagtacctagaacttaaactccagagggcacagccgctaccccaggtgctgggtctcacagcctccccaggcactggcggggcctccaaactcgatggggccatcaaccacgtcctgcagctctgtgccaacttggacacgtggtgcatcatgtcaccccagaactgctgcccccagctgcaggagcacagccaacagccttgcaaacagtacaacctctgccacaggcgcagccaggatccgtttggggacttgctgaagaagctcatggaccaaatccatgaccacctggagatgcctgagttgagccggaaatttgggacgcaaatgtatgagcagcaggtggtgaagctgagtgaggctgcggctttggctgggcttcaggagcaacgggtgtatgcgcttcacctgaggcgctacaatgacgcgctgctcatccatgacaccgtccgcgccgtggatgccttggctgcgctgcaggatttctatcacagggagcacgtcactaaaacccagatcctgtgtgccgagcgccggctgctggccctgttcgatgaccgcaagaatgagctggcccacttggcaactcatggcccagagaatccaaaactggagatgctggaaaagatcctgcaaaggcagttcagtagctctaacagccctcggggtatcatcttcacccgcacccgccaaagcgcacactccctcctgctctggctccagcagcagcagggcctgcagactgtggacatccgggcccagctactgattggggctgggaacagcagccagagcacccacatgacccagagggaccagcaagaagtgatccagaagttccaagatggaaccctgaaccttctggtggccacgagtgtggcggaggaggggctggacatcccacattgcaatgtggtggtgcgttatgggctcttgaccaatgaaatctccatggtccaggccaggggccgtgcccgggccgatcagagtgtatacgcgtttgtagcaactgaaggtagccgggagctgaagcgggagctgatcaacgaggcgctggagacgctgatggagcaggcagtggctgctgtgcagaaaatggaccaggccgagtaccaggccaagatccgggatctgcagcaggcagccttgaccaagcgggcggcccaggcagcccagcgggagaaccagcggcagcagttcccagtggagcacgtgcagctactctgcatcaactgcatggtggctgtgggccatggcagcgacctgcggaaggtggagggcacccaccatgtcaatgtgaaccccaacttctcgaactactataatgtctccagggatcctgtggtcatcaacaaagtcttcaaggactggaagcctgggggtgtcatcagctgcaggaactgtggggaggtctggggtctgcagatgatctacaagtcagtgaagctgccagtgctcaaagtccgcagcatgctgctggagacccctcaggggcggatccaggccaaaaagtggtcccgcgtgcccttctccgtgcctgactttgacttcctgcagcattgtgccgagaacttgtcggacctctccctggac

SEQ ID NO: 183 - RNA, 2034 nt, Homo sapiens
auggagcuucgguccuaccaaugggaggugaucaugccugcccuggagggcaagaauaucaucaucuggcugcccacgggugccgggaagacccgggcggcugcuuauguggccaagcggcaccuagagacuguggauggagccaaggugguuguauuggucaacagggugcaccuggugacccagcauggugaagaguucaggcgcaugcuggauggacgcuggaccgugacaacccugaguggggacaugggaccacgugcuggcuuuggccaccuggcccggugccaugaccugcucaucugcacagcagagcuucugcagauggcacugaccagccccgaggaggaggagcacguggagcucacugucuucucccugaucgugguggaugagugccaccacacgcacaaggacaccgucuacaacgucaucaugagccaguaccuagaacuuaaacuccagagggcacagccgcuaccccaggugcugggucucacagccuccccaggcacuggcggggccuccaaacucgauggggccaucaaccacguccugcagcucugugccaacuuggacacguggugcaucaugucaccccagaacugcugcccccagcugcaggagcacagccaacagccuugcaaacaguacaaccucugccacaggcgcagccaggauccguuuggggacuugcugaagaagcucauggaccaaauccaugaccaccuggagaugccugaguugagccggaaauuugggacgcaaauguaugagcagcagguggugaagcugagugaggcugcggcuuuggcugggcuucaggagcaacggguguaugcgcuucaccugaggcgcuacaaugacgcgcugcucauccaugacaccguccgcgccguggaugccuuggcugcgcugcaggauuucuaucacagggagcacgucacuaaaacccagauccugugugccgagcgccggcugcuggcccuguucgaugaccgcaagaaugagcuggcccacuuggcaacucauggcccagagaauccaaaacuggagaugcuggaaaagauccugcaaaggcaguucaguagcucuaacagcccucgggguaucaucuucacccgcacccgccaaagcgcacacucccuccugcucuggcuccagcagcagcagggccugcagacuguggacauccgggcccagcuacugauuggggcugggaacagcagccagagcacccacaugacccagagggaccagcaagaagugauccagaaguuccaagauggaacccugaaccuucugguggccacgaguguggcggaggaggggcuggacaucccacauugcaaugugguggugcguuaugggcucuugaccaaugaaaucuccaugguccaggccaggggccgugcccgggccgaucagaguguauacgcguuuguagcaacugaagguagccgggagcugaagcgggagcugaucaacgaggcgcuggagacgcugauggagcaggcaguggcugcugugcagaaaauggaccaggccgaguaccaggccaagauccgggaucugcagcaggcagccuugaccaagcgggcggcccaggcagcccagcgggagaaccagcggcagcaguucccaguggagcacgugcagcuacucugcaucaacugcaugguggcugugggccauggcagcgaccugcggaagguggagggcacccaccaugucaaugugaaccccaacuucucgaacuacuauaaugucuccagggauccuguggucaucaacaaagucuucaaggacuggaagccugggggugucaucagcugcaggaacuguggggaggucuggggucugcagaugaucuacaagucagugaagcugccagugcucaaaguccgcagcaugcugcuggagaccccucaggggcggauccaggccaaaaaguggucccgcgugcccuucuccgugccugacuuugacuuccugcagcauugugccgagaacuugucggaccucucccuggac

SEQ ID NO: 184 - DNA, 2037 bp, Artificial Sequence
  Codon optimised DNA encoding LGP2
atggaactgcggagctaccagtgggaagtgatcatgcctgctctggaaggcaagaacatcatcatctggctgcccaccggcgctggcaaaacaagagctgctgcctacgtggccaagcggcacctggaaacagtggatggcgctaaggtggtggtgctggtcaacagagtgcacctggttacccagcacggcgaggaattcagaagaatgctggacggccggtggaccgtgacaacactgtctggcgatatgggccctagagccggctttggacacctggccagatgccacgatctgctgatctgtacagccgaactgctgcagatggccctgacaagccctgaggaagaggaacacgtcgagctgaccgtgttcagcctgatcgtggtggacgagtgccaccacacacacaaggacaccgtgtacaacgtgatcatgagccagtacctggaactgaagctgcagagagcccagcctctgcctcaagtgctgggactgacagcctctcctggaacaggcggagcctctaaactggacggcgccatcaatcacgtgctgcagctgtgcgccaacctggatacctggtgcatcatgtccccacagaactgctgtccccagctgcaagagcactctcagcagccctgcaagcagtacaacctgtgccacagaagatctcaggaccccttcggcgacctgctgaagaaactgatggaccagatccacgaccacctcgagatgcccgagctgagcagaaagttcggcacccagatgtacgagcagcaggttgtgaagctgagcgaagccgctgctctggccggactgcaagaacagagagtgtacgccctgcacctgaggcggtacaatgatgccctgctgatccacgataccgtgcgcgctgttgatgctctggctgctctgcaggatttctaccaccgcgagcacgtgaccaagacacagatcctgtgtgccgagagaaggctgctggccctgttcgacgacagaaagaatgagctggcccacctggctacacacggccccgaaaatcccaagctggaaatgctggaaaagatcctgcagcggcagttcagcagcagcaacagccctagaggcatcatcttcacccggaccagacagagcgcccactctctgctgctgtggctgcagcaacaacagggactgcagaccgtggacattagggcccagctgctgatcggagccggcaatagctctcagagcacccacatgacccagcgggaccagcaagaagtgatccagaagttccaggacggcaccctgaatctgctggtggccacatctgtggctgaggaaggcctggatatccctcactgcaacgtggtcgtcagatacggcctgctgaccaacgagatcagcatggtgcaggccagaggcagagccagagccgatcagtctgtgtacgccttcgtggctacagagggctccagagagctgaagcgcgagctgatcaatgaggccctggaaaccctgatggaacaagccgtggccgccgtgcagaaaatggatcaggccgagtaccaggccaagatcagggatctgcaacaggccgctctgaccaagagagctgctcaggctgcccagagagagaaccagagacagcaattccccgtggaacacgtgcagctgctgtgtatcaactgcatggtggccgtcggacacggcagcgatctgagaaaagtggaaggcacccaccacgtgaacgtgaaccccaacttcagcaactactacaacgtgtccagagatcccgtggtcatcaacaaggtgttcaaggactggaagcctggcggcgtgatcagctgcagaaattgcggagaagtgtggggcctgcagatgatctacaagagcgtgaagctgcccgtgctgaaagtgcggagcatgctgctggaaacaccccagggaagaatccaggccaaaaagtggtccagagtgcccttcagcgtgcccgacttcgatttcctgcagcactgcgccgagaacctgagcgatctgtccctggattga

SEQ ID NO: 185 - RNA, 2037 nt, Artificial Sequence
  Codon optimised RNA encoding LGP2
auggaacugcggagcuaccagugggaagugaucaugccugcucuggaaggcaagaacaucaucaucuggcugcccaccggcgcuggcaaaacaagagcugcugccuacguggccaagcggcaccuggaaacaguggauggcgcuaaggugguggugcuggucaacagagugcaccugguuacccagcacggcgaggaauucagaagaaugcuggacggccgguggaccgugacaacacugucuggcgauaugggcccuagagccggcuuuggacaccuggccagaugccacgaucugcugaucuguacagccgaacugcugcagauggcccugacaagcccugaggaagaggaacacgucgagcugaccguguucagccugaucgugguggacgagugccaccacacacacaaggacaccguguacaacgugaucaugagccaguaccuggaacugaagcugcagagagcccagccucugccucaagugcugggacugacagccucuccuggaacaggcggagccucuaaacuggacggcgccaucaaucacgugcugcagcugugcgccaaccuggauaccuggugcaucauguccccacagaacugcuguccccagcugcaagagcacucucagcagcccugcaagcaguacaaccugugccacagaagaucucaggaccccuucggcgaccugcugaagaaacugauggaccagauccacgaccaccucgagaugcccgagcugagcagaaaguucggcacccagauguacgagcagcagguugugaagcugagcgaagccgcugcucuggccggacugcaagaacagagaguguacgcccugcaccugaggcgguacaaugaugcccugcugauccacgauaccgugcgcgcuguugaugcucuggcugcucugcaggauuucuaccaccgcgagcacgugaccaagacacagauccugugugccgagagaaggcugcuggcccuguucgacgacagaaagaaugagcuggcccaccuggcuacacacggccccgaaaaucccaagcuggaaaugcuggaaaagauccugcagcggcaguucagcagcagcaacagcccuagaggcaucaucuucacccggaccagacagagcgcccacucucugcugcuguggcugcagcaacaacagggacugcagaccguggacauuagggcccagcugcugaucggagccggcaauagcucucagagcacccacaugacccagcgggaccagcaagaagugauccagaaguuccaggacggcacccugaaucugcugguggccacaucuguggcugaggaaggccuggauaucccucacugcaacguggucgucagauacggccugcugaccaacgagaucagcauggugcaggccagaggcagagccagagccgaucagucuguguacgccuucguggcuacagagggcuccagagagcugaagcgcgagcugaucaaugaggcccuggaaacccugauggaacaagccguggccgccgugcagaaaauggaucaggccgaguaccaggccaagaucagggaucugcaacaggccgcucugaccaagagagcugcucaggcugcccagagagagaaccagagacagcaauuccccguggaacacgugcagcugcuguguaucaacugcaugguggccgucggacacggcagcgaucugagaaaaguggaaggcacccaccacgugaacgugaaccccaacuucagcaacuacuacaacguguccagagaucccguggucaucaacaagguguucaaggacuggaagccuggcggcgugaucagcugcagaaauugcggagaaguguggggccugcagaugaucuacaagagcgugaagcugcccgugcugaaagugcggagcaugcugcuggaaacaccccagggaagaauccaggccaaaaagugguccagagugcccuucagcgugcccgacuucgauuuccugcagcacugcgccgagaaccugagcgaucugucccuggauuga

SEQ ID NO: 186 - protein, 880 aa, Homo sapiens
MTTEQRRSLQAFQDYIRKTLDPTYILSYMAPWFREGYSGLYEAIESWDFKKIEKLEEYRLLLKRLQPEFKTRIIPTDIISDLSECLINQECEEILQICSTKGMMAGAEKLVECLLRSDKENWPKTLKLALEKERNKFSELWIVEKGIKDVETEDLEDKMETSDIQIFYQEDPECQNLSENSCPPSEVSDTNLYSPFKPRNYQLELALPAMKGKNTIICAPTGCGKTFVSLLICEHHLKKFPQGQKGKVVFFANQIPVYEQQKSVFSKYFERHGYRVTGISGATAENVPVEQIVENNDIIILTPQILVNNLKKGTIPSLSIFTLMIFDECHNTSKQHPYNMIMFNYLDQKLGGSSGPLPQVIGLTASVGVGDAKNTDEALDYICKLCASLDASVIATVKHNLEELEQVVYKPQKFFRKVESRISDKFKYIIAQLMRDTESLAKRICKDLENLSQIQNREFGTQKYEQWIVTVQKACMVFQMPDKDEESRICKALFLYTSHLRKYNDALIISEHARMKDALDYLKDFFSNVRAAGFDEIEQDLTQRFEEKLQELESVSRDPSNENPKLEDLCFILQEEYHLNPETITILFVKTRALVDALKNWIEGNPKLSFLKPGILTGRGKTNQNTGMTLPAQKCILDAFKASGDHNILIATSVADEGIDIAQCNLVILYEYVGNVIKMIQTRGRGRARGSKCFLLTSNAGVIEKEQINMYKEKMMNDSILRLQTWDEAVFREKILHIQTHEKFIRDSQEKPKPVPDKENKKLLCRKCKALACYTADVRVIEECHYTVLGDAFKECFVSRPHPKPKQFSSFEKRAKIFCARQNCSHDWGIHVKYKTFEIPVIKIESFVVEDIATGVQTLYSKWKDFHFEKIPFDPAEMSK

SEQ ID NO: 187 - DNA, 2640 bp, Homo sapiens
atgaccaccgagcagcgacgcagcctgcaagccttccaggattatatccggaagaccctggaccctacctacatcctgagctacatggccccctggtttagggagggttattctggactttatgaagccattgaaagttgggatttcaaaaaaattgaaaagttggaggagtatagattacttttaaaacgtttacaaccagaatttaaaaccagaattatcccaaccgatatcatttctgatctgtctgaatgtttaattaatcaggaatgtgaagaaattctacagatttgctctactaaggggatgatggcaggtgcagagaaattggtggaatgccttctcagatcagacaaggaaaactggcccaaaactttgaaacttgctttggagaaagaaaggaacaagttcagtgaactgtggattgtagagaaaggtataaaagatgttgaaacagaagatcttgaggataagatggaaacttctgacatacagattttctaccaagaagatccagaatgccagaatcttagtgagaattcatgtccaccttcagaagtgtctgatacaaacttgtacagcccatttaaaccaagaaattaccaattagagcttgctttgcctgctatgaaaggaaaaaacacaataatatgtgctcctacaggttgtggaaaaacctttgtttcactgcttatatgtgaacatcatcttaaaaaattcccacaaggacaaaaggggaaagttgtcttttttgcgaatcagatcccagtgtatgaacagcagaaatctgtattctcaaaatactttgaaagacatgggtatagagttacaggcatttctggagcaacagctgagaatgtcccagtggaacagattgttgagaacaatgacatcatcattttaactccacagattcttgtgaacaaccttaaaaagggaacgattccatcactatccatctttactttgatgatatttgatgaatgccacaacactagtaaacaacacccgtacaatatgatcatgtttaattatctagatcagaaacttggaggatcttcaggcccactgccccaggtcattgggctgactgcctcggttggtgttggggatgccaaaaacacagatgaagccttggattatatctgcaagctgtgtgcttctcttgatgcgtcagtgatagcaacagtcaaacacaatctggaggaactggagcaagttgtttataagccccagaagtttttcaggaaagtggaatcacggattagcgacaaatttaaatacatcatagctcagctgatgagggacacagagagtctggcaaagagaatctgcaaagacctcgaaaacttatctcaaattcaaaatagggaatttggaacacagaaatatgaacaatggattgttacagttcagaaagcatgcatggtgttccagatgccagacaaagatgaagagagcaggatttgtaaagccctgtttttatacacttcacatttgcggaaatataatgatgccctcattatcagtgagcatgcacgaatgaaagatgctctggattacttgaaagacttcttcagcaatgtccgagcagcaggattcgatgagattgagcaagatcttactcagagatttgaagaaaagctgcaggaactagaaagtgtttccagggatcccagcaatgagaatcctaaacttgaagacctctgcttcatcttacaagaagagtaccacttaaacccagagacaataacaattctctttgtgaaaaccagagcacttgtggacgctttaaaaaattggattgaaggaaatcctaaactcagttttctaaaacctggcatattgactggacgtggcaaaacaaatcagaacacaggaatgaccctcccggcacagaagtgtatattggatgcattcaaagccagtggagatcacaatattctgattgccacctcagttgctgatgaaggcattgacattgcacagtgcaatcttgtcatcctttatgagtatgtgggcaatgtcatcaaaatgatccaaaccagaggcagaggaagagcaagaggtagcaagtgcttccttctgactagtaatgctggtgtaattgaaaaagaacaaataaacatgtacaaagaaaaaatgatgaatgactctattttacgccttcagacatgggacgaagcagtatttagggaaaagattctgcatatacagactcatgaaaaattcatcagagatagtcaagaaaaaccaaaacctgtacctgataaggaaaataaaaaactgctctgcagaaagtgcaaagccttggcatgttacacagctgacgtaagagtgatagaggaatgccattacactgtgcttggagatgcttttaaggaatgctttgtgagtagaccacatcccaagccaaagcagttttcaagttttgaaaaaagagcaaagatattctgtgcccgacagaactgcagccatgactggggaatccatgtgaagtacaagacatttgagattccagttataaaaattgaaagttttgtggtggaggatattgcaactggagttcagacactgtactcgaagtggaaggactttcattttgagaagataccatttgatccagcagaaatgtccaaa

SEQ ID NO: 188 - RNA, 2640 nt, Homo sapiens
augaccaccgagcagcgacgcagccugcaagccuuccaggauuauauccggaagacccuggacccuaccuacauccugagcuacauggcccccugguuuagggaggguuauucuggacuuuaugaagccauugaaaguugggauuucaaaaaaauugaaaaguuggaggaguauagauuacuuuuaaaacguuuacaaccagaauuuaaaaccagaauuaucccaaccgauaucauuucugaucugucugaauguuuaauuaaucaggaaugugaagaaauucuacagauuugcucuacuaaggggaugauggcaggugcagagaaauugguggaaugccuucucagaucagacaaggaaaacuggcccaaaacuuugaaacuugcuuuggagaaagaaaggaacaaguucagugaacuguggauuguagagaaagguauaaaagauguugaaacagaagaucuugaggauaagauggaaacuucugacauacagauuuucuaccaagaagauccagaaugccagaaucuuagugagaauucauguccaccuucagaagugucugauacaaacuuguacagcccauuuaaaccaagaaauuaccaauuagagcuugcuuugccugcuaugaaaggaaaaaacacaauaauaugugcuccuacagguuguggaaaaaccuuuguuucacugcuuauaugugaacaucaucuuaaaaaauucccacaaggacaaaaggggaaaguugucuuuuuugcgaaucagaucccaguguaugaacagcagaaaucuguauucucaaaauacuuugaaagacauggguauagaguuacaggcauuucuggagcaacagcugagaaugucccaguggaacagauuguugagaacaaugacaucaucauuuuaacuccacagauucuugugaacaaccuuaaaaagggaacgauuccaucacuauccaucuuuacuuugaugauauuugaugaaugccacaacacuaguaaacaacacccguacaauaugaucauguuuaauuaucuagaucagaaacuuggaggaucuucaggcccacugccccaggucauugggcugacugccucgguugguguuggggaugccaaaaacacagaugaagccuuggauuauaucugcaagcugugugcuucucuugaugcgucagugauagcaacagucaaacacaaucuggaggaacuggagcaaguuguuuauaagccccagaaguuuuucaggaaaguggaaucacggauuagcgacaaauuuaaauacaucauagcucagcugaugagggacacagagagucuggcaaagagaaucugcaaagaccucgaaaacuuaucucaaauucaaaauagggaauuuggaacacagaaauaugaacaauggauuguuacaguucagaaagcaugcaugguguuccagaugccagacaaagaugaagagagcaggauuuguaaagcccuguuuuuauacacuucacauuugcggaaauauaaugaugcccucauuaucagugagcaugcacgaaugaaagaugcucuggauuacuugaaagacuucuucagcaauguccgagcagcaggauucgaugagauugagcaagaucuuacucagagauuugaagaaaagcugcaggaacuagaaaguguuuccagggaucccagcaaugagaauccuaaacuugaagaccucugcuucaucuuacaagaagaguaccacuuaaacccagagacaauaacaauucucuuugugaaaaccagagcacuuguggacgcuuuaaaaaauuggauugaaggaaauccuaaacucaguuuucuaaaaccuggcauauugacuggacguggcaaaacaaaucagaacacaggaaugacccucccggcacagaaguguauauuggaugcauucaaagccaguggagaucacaauauucugauugccaccucaguugcugaugaaggcauugacauugcacagugcaaucuugucauccuuuaugaguaugugggcaaugucaucaaaaugauccaaaccagaggcagaggaagagcaagagguagcaagugcuuccuucugacuaguaaugcugguguaauugaaaaagaacaaauaaacauguacaaagaaaaaaugaugaaugacucuauuuuacgccuucagacaugggacgaagcaguauuuagggaaaagauucugcauauacagacucaugaaaaauucaucagagauagucaagaaaaaccaaaaccuguaccugauaaggaaaauaaaaaacugcucugcagaaagugcaaagccuuggcauguuacacagcugacguaagagugauagaggaaugccauuacacugugcuuggagaugcuuuuaaggaaugcuuugugaguagaccacaucccaagccaaagcaguuuucaaguuuugaaaaaagagcaaagauauucugugcccgacagaacugcagccaugacuggggaauccaugugaaguacaagacauuugagauuccaguuauaaaaauugaaaguuuugugguggaggauauugcaacuggaguucagacacuguacucgaaguggaaggacuuucauuuugagaagauaccauuugauccagcagaaauguccaaa

SEQ ID NO: 189 - DNA, 2643 bp, Artificial Sequence
  Codon optimised DNA encoding RIG splice variant
atgaccaccgagcagagaagatccctgcaggccttccaggactacatcagaaagacactggaccccacctacatcctgagctacatggccccatggttcagagagggctacagcggactgtacgaggccatcgagagctgggacttcaagaagatcgagaagctggaagagtaccggctgctgctgaagagactgcagcccgagttcaagacccggatcatccccaccgacatcatcagcgatctgagcgagtgcctgatcaatcaagagtgcgaggaaatcctgcagatctgtagcaccaagggcatgatggctggcgccgagaaactggtggaatgcctgctgagaagcgacaaagagaactggcccaagacactgaagctggccctggaaaaagagcggaacaagttcagcgagctgtggatcgtggaaaagggcatcaaggacgtggaaaccgaggacctggaagataagatggaaaccagcgacatccagatcttctaccaagaggaccccgagtgccagaacctgagcgagaatagctgccctcctagcgaggtgtccgacaccaatctgtacagccccttcaagccccggaactaccagctggaacttgccctgcctgccatgaagggcaagaacaccatcatctgtgccccaaccggctgcggcaagacctttgtgtctctgctgatctgcgagcaccacctgaagaagttccctcagggccagaaaggcaaggtggtgtttttcgccaatcagatccccgtgtacgagcagcagaaaagcgtgttcagcaagtacttcgagcggcacggctacagagtgacaggcatttctggcgccaccgccgagaatgtgcctgtggaacagattgtggaaaacaacgatatcatcatcctgacgcctcagatcctggtcaacaatctgaagaagggcacaatccccagcctgagcatcttcaccctgatgatcttcgacgagtgccacaacaccagcaagcagcacccctacaatatgatcatgttcaactacctggaccagaagctcggcggcagctctggacctctgcctcaagtgattggcctgacagcctctgtcggagtgggcgacgccaagaatactgacgaggccctggattacatctgcaagctgtgcgccagcctggacgcctctgtgattgccaccgtgaagcacaacctcgaggaactggaacaggtggtgtacaagccccagaaattctttcggaaggtggaaagccggatcagcgacaagttcaagtacatcattgcccagctgatgcgggacaccgagagcctggctaagagaatctgcaaggatctggaaaacctgagccagatccagaacagagagttcggcacccagaaatacgagcagtggattgtgaccgtgcagaaagcctgcatggtgttccagatgcctgacaaggacgaagagagccggatctgcaaagccctgttcctgtacaccagccacctgagaaagtacaacgacgccctgatcatctccgagcacgccagaatgaaggacgccctggactacctgaaggacttcttctccaatgtgcgcgctgccggcttcgatgagatcgagcaagatctgacccagcgcttcgaggaaaagctgcaagagctggaaagcgtgtccagagatcccagcaacgagaaccccaaactggaagatctgtgcttcatcctgcaagaggaataccatctgaaccccgagacaatcaccatcctgttcgtgaaaacaagagccctggtggatgccctgaagaactggatcgagggcaaccccaagctgagcttcctgaagcctggcatcctgaccggcagaggcaagacaaaccagaacaccggcatgaccctgccagctcagaagtgcatcctggacgcttttaaggccagcggcgaccacaacatcctgatcgccacatctgtggccgacgagggcatcgatatcgcccagtgcaatctggtcatcctgtacgagtacgtgggcaacgtgatcaagatgatccagacaagaggcaggggcagagccagaggcagcaagtgctttctgctgacctctaatgccggcgtgatcgagaaagaacagatcaacatgtacaaagaaaagatgatgaacgacagcatcctgcggctgcagacctgggatgaagccgtgttccgggaaaagatcctgcacatccagacacacgagaagttcatccgggacagccaagagaagcccaagcctgtgcctgacaaagaaaacaagaaactgctgtgccggaagtgcaaggccctggcctgttatacagccgacgtgcgagtgatcgaggaatgccactataccgtgctcggcgacgccttcaaagaatgcttcgtgtcccggcctcatcctaagcctaagcagttcagcagcttcgagaagcgggccaagatcttctgcgccagacagaactgcagccacgactggggaatccacgtgaagtacaagaccttcgagatcccggtcatcaagatcgagtccttcgtggtggaagatatcgccaccggcgtgcagaccctgtacagcaagtggaaggatttccacttcgagaaaatccctttcgaccccgccgagatgagcaagtga

SEQ ID NO: 190 - RNA, 2643 nt, Artificial Sequence
  Codon optimised RNA encoding RIG splice variant
augaccaccgagcagagaagaucccugcaggccuuccaggacuacaucagaaagacacuggaccccaccuacauccugagcuacauggccccaugguucagagagggcuacagcggacuguacgaggccaucgagagcugggacuucaagaagaucgagaagcuggaagaguaccggcugcugcugaagagacugcagcccgaguucaagacccggaucauccccaccgacaucaucagcgaucugagcgagugccugaucaaucaagagugcgaggaaauccugcagaucuguagcaccaagggcaugauggcuggcgccgagaaacugguggaaugccugcugagaagcgacaaagagaacuggcccaagacacugaagcuggcccuggaaaaagagcggaacaaguucagcgagcuguggaucguggaaaagggcaucaaggacguggaaaccgaggaccuggaagauaagauggaaaccagcgacauccagaucuucuaccaagaggaccccgagugccagaaccugagcgagaauagcugcccuccuagcgagguguccgacaccaaucuguacagccccuucaagccccggaacuaccagcuggaacuugcccugccugccaugaagggcaagaacaccaucaucugugccccaaccggcugcggcaagaccuuugugucucugcugaucugcgagcaccaccugaagaaguucccucagggccagaaaggcaaggugguguuuuucgccaaucagauccccguguacgagcagcagaaaagcguguucagcaaguacuucgagcggcacggcuacagagugacaggcauuucuggcgccaccgccgagaaugugccuguggaacagauuguggaaaacaacgauaucaucauccugacgccucagauccuggucaacaaucugaagaagggcacaauccccagccugagcaucuucacccugaugaucuucgacgagugccacaacaccagcaagcagcaccccuacaauaugaucauguucaacuaccuggaccagaagcucggcggcagcucuggaccucugccucaagugauuggccugacagccucugucggagugggcgacgccaagaauacugacgaggcccuggauuacaucugcaagcugugcgccagccuggacgccucugugauugccaccgugaagcacaaccucgaggaacuggaacaggugguguacaagccccagaaauucuuucggaagguggaaagccggaucagcgacaaguucaaguacaucauugcccagcugaugcgggacaccgagagccuggcuaagagaaucugcaaggaucuggaaaaccugagccagauccagaacagagaguucggcacccagaaauacgagcaguggauugugaccgugcagaaagccugcaugguguuccagaugccugacaaggacgaagagagccggaucugcaaagcccuguuccuguacaccagccaccugagaaaguacaacgacgcccugaucaucuccgagcacgccagaaugaaggacgcccuggacuaccugaaggacuucuucuccaaugugcgcgcugccggcuucgaugagaucgagcaagaucugacccagcgcuucgaggaaaagcugcaagagcuggaaagcguguccagagaucccagcaacgagaaccccaaacuggaagaucugugcuucauccugcaagaggaauaccaucugaaccccgagacaaucaccauccuguucgugaaaacaagagcccugguggaugcccugaagaacuggaucgagggcaaccccaagcugagcuuccugaagccuggcauccugaccggcagaggcaagacaaaccagaacaccggcaugacccugccagcucagaagugcauccuggacgcuuuuaaggccagcggcgaccacaacauccugaucgccacaucuguggccgacgagggcaucgauaucgcccagugcaaucuggucauccuguacgaguacgugggcaacgugaucaagaugauccagacaagaggcaggggcagagccagaggcagcaagugcuuucugcugaccucuaaugccggcgugaucgagaaagaacagaucaacauguacaaagaaaagaugaugaacgacagcauccugcggcugcagaccugggaugaagccguguuccgggaaaagauccugcacauccagacacacgagaaguucauccgggacagccaagagaagcccaagccugugccugacaaagaaaacaagaaacugcugugccggaagugcaaggcccuggccuguuauacagccgacgugcgagugaucgaggaaugccacuauaccgugcucggcgacgccuucaaagaaugcuucgugucccggccucauccuaagccuaagcaguucagcagcuucgagaagcgggccaagaucuucugcgccagacagaacugcagccacgacuggggaauccacgugaaguacaagaccuucgagaucccggucaucaagaucgaguccuucgugguggaagauaucgccaccggcgugcagacccuguacagcaaguggaaggauuuccacuucgagaaaaucccuuucgaccccgccgagaugagcaaguga

SEQ ID NO: 191 - protein, 547 aa, Homo sapiens
MEDSEALGFEHMGLDPRLLQAVTDLGWSRPTLIQEKAIPLALEGKDLLARARTGSGKTAAYAIPMLQLLLHRKATGPVVEQAVRGLVLVPTKELARQAQSMIQQLATYCARDVRVANVSAAEDSVSQRAVLMEKPDVVVGTPSRILSHLQQDSLKLRDSLELLVVDEADLLFSFGFEEELKSLLCHLPRIYQAFLMSATFNEDVQALKELILHNPVTLKLQESQLPGPDQLQQFQVVCETEEDKFLLLYALLKLSLIRGKSLLFVNTLERSYRLRLFLEQFSIPTCVLNGELPLRSRCHIISQFNQGFYDCVIATDAEVLGAPVKGKRRGRGPKGDKASDPEAGVARGIDFHHVSAVLNFDLPPTPEAYIHRAGRTARANNPGIVLTFVLPTEQFHLGKIEELLSGENRGPILLPYQFRMEEIEGFRYRCRDAMRSVTKQAIREARLKEIKEELLHSEKLKTYFEDNPRDLQLLRHDLPLHPAVVKPHLGHVPDYLVPPALRGLVRPHKKRKKLSSSCRKAKRAKSQNPLRSFKHKGKKFRPTAKPS

SEQ ID NO: 192 - DNA, 1641 bp, Homo sapiens
atggaggactctgaagcactgggcttcgaacacatgggcctcgatccccggctccttcaggctgtcaccgatctgggctggtcgcgacctacgctgatccaggagaaggccatcccactggccctagaagggaaggacctcctggctcgggcccgcacgggctccgggaagacggccgcttatgctattccgatgctgcagctgttgctccataggaaggcgacaggtccggtggtagaacaggcagtgagaggccttgttcttgttcctaccaaggagctggcacggcaagcacagtccatgattcagcagctggctacctactgtgctcgggatgtccgagtggccaatgtctcagctgctgaagactcagtctctcagagagctgtgctgatggagaagccagatgtggtagtagggaccccatctcgcatattaagccacttgcagcaagacagcctgaaacttcgtgactccctggagcttttggtggtggacgaagctgaccttcttttttcctttggctttgaagaagagctcaagagtctcctctgtcacttgccccggatttaccaggcttttctcatgtcagctacttttaacgaggacgtacaagcactcaaggagctgatattacataacccggttacccttaagttacaggagtcccagctgcctgggccagaccagttacagcagtttcaggtggtctgtgagactgaggaagacaaattcctcctgctgtatgccctgctcaagctgtcattgattcggggcaagtctctgctctttgtcaacactctagaacggagttaccggctacgcctgttcttggaacagttcagcatccccacctgtgtgctcaatggagagcttccactgcgctccaggtgccacatcatctcacagttcaaccaaggcttctacgactgtgtcatagcaactgatgctgaagtcctgggggccccagtcaagggcaagcgtcggggccgagggcccaaaggggacaaggcctctgatccggaagcaggtgtggcccggggcatagacttccaccatgtgtctgctgtgctcaactttgatcttcccccaacccctgaggcctacatccatcgagctggcaggacagcacgcgctaacaacccaggcatagtcttaacctttgtgcttcccacggagcagttccacttaggcaagattgaggagcttctcagtggagagaacaggggccccattctgctcccctaccagttccggatggaggagatcgagggcttccgctatcgctgcagggatgccatgcgctcagtgactaagcaggccattcgggaggcaagattgaaggagatcaaggaagagcttctgcattctgagaagcttaagacatactttgaagacaaccctagggacctccagctgctgcggcatgacctacctttgcaccccgcagtggtgaagccccacctgggccatgttcctgactacctggttcctcctgctctccgtggcctggtgcgccctcacaagaagcggaagaagctgtcttcctcttgtaggaaggccaagagagcaaagtcccagaacccactgcgcagcttcaagcacaaaggaaagaaattcagacccacagccaagccctcc

SEQ ID NO: 193 - RNA, 1641 nt, Homo sapiens
auggaggacucugaagcacugggcuucgaacacaugggccucgauccccggcuccuucaggcugucaccgaucugggcuggucgcgaccuacgcugauccaggagaaggccaucccacuggcccuagaagggaaggaccuccuggcucgggcccgcacgggcuccgggaagacggccgcuuaugcuauuccgaugcugcagcuguugcuccauaggaaggcgacagguccggugguagaacaggcagugagaggccuuguucuuguuccuaccaaggagcuggcacggcaagcacaguccaugauucagcagcuggcuaccuacugugcucgggauguccgaguggccaaugucucagcugcugaagacucagucucucagagagcugugcugauggagaagccagaugugguaguagggaccccaucucgcauauuaagccacuugcagcaagacagccugaaacuucgugacucccuggagcuuuuggugguggacgaagcugaccuucuuuuuuccuuuggcuuugaagaagagcucaagagucuccucugucacuugccccggauuuaccaggcuuuucucaugucagcuacuuuuaacgaggacguacaagcacucaaggagcugauauuacauaacccgguuacccuuaaguuacaggagucccagcugccugggccagaccaguuacagcaguuucagguggucugugagacugaggaagacaaauuccuccugcuguaugcccugcucaagcugucauugauucggggcaagucucugcucuuugucaacacucuagaacggaguuaccggcuacgccuguucuuggaacaguucagcauccccaccugugugcucaauggagagcuuccacugcgcuccaggugccacaucaucucacaguucaaccaaggcuucuacgacugugucauagcaacugaugcugaaguccugggggccccagucaagggcaagcgucggggccgagggcccaaaggggacaaggccucugauccggaagcagguguggcccggggcauagacuuccaccaugugucugcugugcucaacuuugaucuucccccaaccccugaggccuacauccaucgagcuggcaggacagcacgcgcuaacaacccaggcauagucuuaaccuuugugcuucccacggagcaguuccacuuaggcaagauugaggagcuucucaguggagagaacaggggccccauucugcuccccuaccaguuccggauggaggagaucgagggcuuccgcuaucgcugcagggaugccaugcgcucagugacuaagcaggccauucgggaggcaagauugaaggagaucaaggaagagcuucugcauucugagaagcuuaagacauacuuugaagacaacccuagggaccuccagcugcugcggcaugaccuaccuuugcaccccgcaguggugaagccccaccugggccauguuccugacuaccugguuccuccugcucuccguggccuggugcgcccucacaagaagcggaagaagcugucuuccucuuguaggaaggccaagagagcaaagucccagaacccacugcgcagcuucaagcacaaaggaaagaaauucagacccacagccaagcccucc

SEQ ID NO: 194 - DNA, 1644 bp, Artificial Sequence
  Codon optimised DNA encoding DDX-56
atggaagattctgaggccctgggcttcgagcacatgggccttgatcctagactgctgcaggccgtgacagatctcggatggtccagacctacactgatccaagagaaggccattcctctggctctggaaggcaaggacctgctggccagagctagaacaggctctggcaagacagccgcctacgctatccctatgctgcagctgctgctgcacagaaaggccacaggaccagtggtggaacaggccgttagaggactggtgctggtgcccacaaaagagctggctagacaggcccagagcatgatccagcagctggccacatactgcgccagagatgtgcgagtggccaatgtgtctgccgccgaggattctgtgtctcagagggccgtgctgatggaaaagcccgatgtggtcgtgggcacccctagcagaatcctgtctcatctgcagcaggacagcctgaagctgagagacagcctggaactgctggtggtggatgaggccgatctgctgttcagcttcggcttcgaggaagaactgaagtccctgctgtgccatctgcctcggatctaccaggccttcctgatgagcgccaccttcaacgaagatgtgcaggccctgaaagagctgatcctgcacaaccccgtgacactgaagctgcaagagagccagctgccaggacctgatcagctccagcagtttcaagtcgtgtgcgagacagaagaggacaagttcctgctgctgtacgccctgctgaagctgtccctgatcagaggcaagagcctgctgttcgtgaacaccctggaaagaagctaccggctgcggctgtttctggaacagttcagcatccctacctgcgtgctgaacggcgagctgcctctgagaagcagatgccacatcatcagccagttcaaccagggcttctacgactgcgtgatcgccacagatgccgaagtgctgggagcacccgtgaagggcaaaagaagaggcagaggccccaagggcgataaggccagtgatcctgaagcaggcgtggccagaggcatcgattttcaccatgtgtccgctgtgctgaacttcgacctgccacctacacctgaggcctacatccacagagccggcagaacagccagagccaacaatcctggcatcgtgctgaccttcgtgctgcctaccgaacagttccacctgggcaagatcgaagaactgctgtccggcgagaacaggggccctatcctgctgccttaccagttccggatggaagagatcgagggcttcagatacagatgcagggacgccatgcggagcgtgacaaagcaggccattagagaggcccggctgaaagagatcaaagaggaactgctccacagcgagaagctcaagacctacttcgaggacaaccccagggacctgcagctcctgagacatgatctgcctctgcaccctgccgtggtcaaacctcatctgggacacgtgcccgactacctggttcctcctgctctgagaggccttgtgcgccctcacaagaagcggaagaagctgagcagctcttgtcggaaggccaagcgggccaagagccagaatccactgagaagcttcaagcacaagggcaagaagttcagacccaccgccaagcctagctga

SEQ ID NO: 195 - RNA, 1644 nt, Artificial Sequence
  Codon optimised RNA encoding DDX-56
auggaagauucugaggcccugggcuucgagcacaugggccuugauccuagacugcugcaggccgugacagaucucggaugguccagaccuacacugauccaagagaaggccauuccucuggcucuggaaggcaaggaccugcuggccagagcuagaacaggcucuggcaagacagccgccuacgcuaucccuaugcugcagcugcugcugcacagaaaggccacaggaccagugguggaacaggccguuagaggacuggugcuggugcccacaaaagagcuggcuagacaggcccagagcaugauccagcagcuggccacauacugcgccagagaugugcgaguggccaaugugucugccgccgaggauucugugucucagagggccgugcugauggaaaagcccgauguggucgugggcaccccuagcagaauccugucucaucugcagcaggacagccugaagcugagagacagccuggaacugcuggugguggaugaggccgaucugcuguucagcuucggcuucgaggaagaacugaagucccugcugugccaucugccucggaucuaccaggccuuccugaugagcgccaccuucaacgaagaugugcaggcccugaaagagcugauccugcacaaccccgugacacugaagcugcaagagagccagcugccaggaccugaucagcuccagcaguuucaagucgugugcgagacagaagaggacaaguuccugcugcuguacgcccugcugaagcugucccugaucagaggcaagagccugcuguucgugaacacccuggaaagaagcuaccggcugcggcuguuucuggaacaguucagcaucccuaccugcgugcugaacggcgagcugccucugagaagcagaugccacaucaucagccaguucaaccagggcuucuacgacugcgugaucgccacagaugccgaagugcugggagcacccgugaagggcaaaagaagaggcagaggccccaagggcgauaaggccagugauccugaagcaggcguggccagaggcaucgauuuucaccauguguccgcugugcugaacuucgaccugccaccuacaccugaggccuacauccacagagccggcagaacagccagagccaacaauccuggcaucgugcugaccuucgugcugccuaccgaacaguuccaccugggcaagaucgaagaacugcuguccggcgagaacaggggcccuauccugcugccuuaccaguuccggauggaagagaucgagggcuucagauacagaugcagggacgccaugcggagcgugacaaagcaggccauuagagaggcccggcugaaagagaucaaagaggaacugcuccacagcgagaagcucaagaccuacuucgaggacaaccccagggaccugcagcuccugagacaugaucugccucugcacccugccguggucaaaccucaucugggacacgugcccgacuaccugguuccuccugcucugagaggccuugugcgcccucacaagaagcggaagaagcugagcagcucuugucggaaggccaagcgggccaagagccagaauccacugagaagcuucaagcacaagggcaagaaguucagacccaccgccaagccuagcuga

SEQ ID NO: 196 - protein, 173 aa, Homo sapiens
MCLLLGATGVGKTLLVKRLQEVSSRDGKGDLGEPPPTRPTVGTNLTDIVAQRKITIRELGGCMGPIWSSYYGNCRSLLFVMDASDPTQLSASCVQLLGLLSAEQLAEASVLILFNKIDLPCYMSTEEMKSLIRLPDIIACAKQNITTAEISAREGTGLAGVLAWLQATHRAND

SEQ ID NO: 197 - DNA, 519 bp, Homo sapiens
atgtgtctcctgctgggggccacgggcgtcgggaagacgctgctggtgaaacggctgcaggaggtgagctcccgggatgggaaaggcgacctgggggagccgcccccgacacggcccacggtgggcaccaatcttactgacatcgtggcacagagaaagatcaccatccgggagcttggggggtgcatgggccccatctggtccagttactatggaaactgccgttctctcctgtttgtgatggacgcctctgaccccacccagctctctgcatcctgtgtgcagctcttaggtctcctttctgcagaacaacttgcagaagcatcggtgctgatactcttcaataaaatcgacctaccctgttacatgtccacggaggagatgaagtcattaatcaggcttccagacatcattgcttgtgccaagcagaacatcaccacggcagaaatcagcgcccgtgaaggcactggcttagcaggggtgctggcctggctccaggccacccacagagccaacgat

SEQ ID NO: 198 - RNA, 519 nt, Homo sapiens
augugucuccugcugggggccacgggcgucgggaagacgcugcuggugaaacggcugcaggaggugagcucccgggaugggaaaggcgaccugggggagccgcccccgacacggcccacggugggcaccaaucuuacugacaucguggcacagagaaagaucaccauccgggagcuuggggggugcaugggccccaucugguccaguuacuauggaaacugccguucucuccuguuugugauggacgccucugaccccacccagcucucugcauccugugugcagcucuuaggucuccuuucugcagaacaacuugcagaagcaucggugcugauacucuucaauaaaaucgaccuacccuguuacauguccacggaggagaugaagucauuaaucaggcuuccagacaucauugcuugugccaagcagaacaucaccacggcagaaaucagcgcccgugaaggcacuggcuuagcaggggugcuggccuggcuccaggccacccacagagccaacgau

SEQ ID NO: 199 - protein, 178 aa, Homo sapiens
MGLIFAKLWSLFCNQEHKVIIVGLDNAGKTTIYQFLMNEVVHTSPTIGSNVEEIVVKNTHFLMWDIGGQESLRSSWNTYYSNTEFIILVVDSIDRERLAITKEELYRMLAHEDLRKAAVLIFANKQDMKGCMTAAEISKYLTLSSIKDHPWHIQSCCALTGEGLCQGLEWMTSRIGVR

SEQ ID NO: 200 - DNA, 537 bp, Homo sapiens
atggggctgatcttcgccaaactgtggagcctcttctgtaaccaagaacacaaagtaattatagtgggactggataatgcagggaaaaccaccattctttaccaattcttaatgaatgaagtggttcatacttctccaaccataggaagcaatgttgaagaaatagttgtgaagaacactcattttcttatgtgggatattggtggtcaggagtctctgcgatcatcctggaacacatattactcaaatacagagttcatcattcttgttgttgatagcattgacagggaacgactagctattacaaaagaagaattatacagaatgttggctcatgaggatttacggaaggctgcagtccttatctttgcaaataaacaggatatgaaagggtgtatgacagcagctgaaatctcgaaatacctcacccttagttcaattaaggatcatccatggcacattcaatcctgctgtgctctcacaggagaagggttatgccaaggtctagagtggatgacctcccggattggtgtgaga

SEQ ID NO: 201 - RNA, 537 nt, Homo sapiens
auggggcugaucuucgccaaacuguggagccucuucuguaaccaagaacacaaaguaauuauagugggacuggauaaugcagggaaaaccaccauucuuuaccaauucuuaaugaaugaagugguucauacuucuccaaccauaggaagcaauguugaagaaauaguugugaagaacacucauuuucuuaugugggauauugguggucaggagucucugcgaucauccuggaacacauauuacucaaauacagaguucaucauucuuguuguugauagcauugacagggaacgacuagcuauuacaaaagaagaauuauacagaauguuggcucaugaggauuuacggaaggcugcaguccuuaucuuugcaaauaaacaggauaugaaaggguguaugacagcagcugaaaucucgaaauaccucacccuuaguucaauuaaggaucauccauggcacauucaauccugcugugcucucacaggagaaggguuaugccaaggucuagaguggaugaccucccggauuggugugaga

SEQ ID NO: 202 - DNA, 540 bp, Artificial Sequence
  Codon optimised DNA encoding ARL5B
atgggcctgatcttcgccaaactgtggtccctgttctgcaatcaagagcacaaagtgatcatcgtcggcctggacaacgccggcaagacaacaatcctgtaccagttcctgatgaacgaggtggtgcacacaagccccaccatcggcagcaacgtggaagagatcgtggtcaagaatacccacttcctgatgtgggacatcggcggccaagagagcctgagaagcagctggaacacctactacagcaacaccgagttcatcatcctggtggtggacagcatcgacagagagagactggccatcaccaaagaggaactgtaccggatgctggcccacgaggatctgagaaaagccgccgtgctgatttttgccaacaagcaggacatgaagggctgcatgacagccgccgagatcagcaagtacctgacactgagcagcatcaaggatcacccctggcacatccagagctgctgtgcattgacaggcgagggcctgtgtcagggactcgagtggatgacaagcagaatcggcgtgcggtga

SEQ ID NO: 203 - RNA, 540 nt, Artificial Sequence
  Codon optimised RNA encoding ARL5B
augggccugaucuucgccaaacuguggucccuguucugcaaucaagagcacaaagugaucaucgucggccuggacaacgccggcaagacaacaauccuguaccaguuccugaugaacgagguggugcacacaagccccaccaucggcagcaacguggaagagaucguggucaagaauacccacuuccugaugugggacaucggcggccaagagagccugagaagcagcuggaacaccuacuacagcaacaccgaguucaucauccuggugguggacagcaucgacagagagagacuggccaucaccaaagaggaacuguaccggaugcuggcccacgaggaucugagaaaagccgccgugcugauuuuugccaacaagcaggacaugaagggcugcaugacagccgccgagaucagcaaguaccugacacugagcagcaucaaggaucaccccuggcacauccagagcugcugugcauugacaggcgagggccugugucagggacucgaguggaugacaagcagaaucggcgugcgguga

SEQ ID NO: 204 - DNA, 45 bp, Artificial Sequence
  26S promoter
gggcccctataactctctacggctaacctgaatggactacgacat

SEQ ID NO: 205 - protein, 19 aa, Artificial Sequence
  P2A spacer
ATNFSLLKQAGDVEENPGP

SEQ ID NO: 206 - protein, 20 aa, Artificial Sequence
  T2A spacer
QCTNYALLKLAGDVESNPGP

SEQ ID NO: 207 - protein, 22 aa, Artificial Sequence
  F2A spacer
VKQTLNFDLLKLAGDVESNPGP

SEQ ID NO: 208 - protein, 6 aa, Artificial Sequence
  Furin sequence
  misc_feature (2)..(2): Xaa can be any naturally occurring amino acid
  misc_feature (5)..(5): Xaa can be any naturally occurring amino acid
RXRKXR

SEQ ID NO: 209 - protein, 6 aa, Artificial Sequence
  Furin sequence
RRRRRR

SEQ ID NO: 210 - protein, 27 aa, Artificial Sequence
  Furin/T2A sequence
RRRRRRGSGEGRGSLLTCGDVEENPGP

SEQ ID NO: 211 - DNA, 461 bp, Foot-and-mouth disease virus
agcaggtttccccaactgacacaaaacgtgcaacttgaaactccgcctggtctttccaggtctagaggggtaacactttgtactgcgtttggctccacgctcgatccactggcgagtgttagtaacagcactgttgcttcgtagcggagcatgacggccgtgggaactcctccttggtaacaaggacccacggggccaaaagccacgcccacacgggcccgtcatgtgtgcaaccccagcacggcgactttactgcgaaacccactttaaagtgacattgaaactggtacccacacactggtgacaggctaaggatgcccttcaggtaccccgaggtaacacgcgacactcgggatctgagaaggggactggggcttctataaaagcgctcggtttaaaaagcttctatgcctgaataggtgaccggaggtcggcacctttcctttgcaattactgaccac

SEQ ID NO: 212 - DNA, 551 bp, Encephalomyocarditis virus
cgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctcccctcaagcgtattcaacaaggggctgaaggatgcccagaaggtaccccattgtatgggatctgatctggggcctcggtgcacatgcttttcatgtgtttagtcgaggttaaaaaacgtctaggccccccgaaccacggggacgtggttttcctttgaaaaacacgatgataata

SEQ ID NO: 213 - DNA, 45 bp, Artificial Sequence
  Flexible linker
ggaggtggcgggtccgggggcgggggtagcggtggcgggggctcc

SEQ ID NO: 214 - protein, 15 aa, Artificial Sequence
  Flexible linker
GGGGSGGGGSGGGGS

SEQ ID NO: 215 - protein, 535 aa, Unknown
  Non-structural protein 1
MEKVHVDIEEDSPFLRALQRSFPQFEVEAKQVTDNDHANARAFSHLASKLIETEVDPSDTILDIGSAPARRMYSKHKYHCICPMRCAEDPDRLYKYATKLKKNCKEITDKELDKKMKELAAVMSDPDLETETMCLHDDESCRYEGQVAVYQDVYAVDGPTSLYHQANKGVRVAYWIGFDTTPFMFKNLAGAYPSYSTNWADETVLTARNIGLCSSDVMERSRRGMSILRKKYLKPSNNVLFSVGSTIYHEKRDLLRSWHLPSVFHLRGKQNYTCRCETIVSCDGYVVKRIAISPGLYGKPSGYAATMHREGFLCCKVTDTLNGERVSFPVCTYVPATLCDQMTGILATDVSADDAQKLLVGLNQRIVVNGRTQRNTNTMKNYLLPVVAQAFARWAKEYKEDQEDERPLGLRDRQLVMGCCWAFRRHKITSIYKRPDTQTIIKVNSDFHSFVLPRIGSNTLEIGLRTRIRKMLEEHKEPSPLITAEDVQEAKCAADEAKEVREAEELRAALPPLAADVEEPTLEADVDLMLQEAGA

SEQ ID NO: 216 - DNA, 1605 bp, Unknown
  Non-structural protein 1
atggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggcc

SEQ ID NO: 217 - RNA, 1605 nt, Unknown
  Non-structural protein 1
auggagaaaguucacguugacaucgaggaagacagcccauuccucagagcuuugcagcggagcuucccgcaguuugagguagaagccaagcaggucacugauaaugaccaugcuaaugccagagcguuuucgcaucuggcuucaaaacugaucgaaacggagguggacccauccgacacgauccuugacauuggaagugcgcccgcccgcagaauguauucuaagcacaaguaucauuguaucuguccgaugagaugugcggaagauccggacagauuguauaaguaugcaacuaagcugaagaaaaacuguaaggaaauaacugauaaggaauuggacaagaaaaugaaggagcuggccgccgucaugagcgacccugaccuggaaacugagacuaugugccuccacgacgacgagucgugucgcuacgaagggcaagucgcuguuuaccaggauguauacgcgguugacggaccgacaagucucuaucaccaagccaauaagggaguuagagucgccuacuggauaggcuuugacaccaccccuuuuauguuuaagaacuuggcuggagcauauccaucauacucuaccaacugggccgacgaaaccguguuaacggcucguaacauaggccuaugcagcucugacguuauggagcggucacguagagggauguccauucuuagaaagaaguauuugaaaccauccaacaauguucuauucucuguuggcucgaccaucuaccacgagaagagggacuuacugaggagcuggcaccugccgucuguauuucacuuacguggcaagcaaaauuacacaugucggugugagacuauaguuaguugcgacggguacgucguuaaaagaauagcuaucaguccaggccuguaugggaagccuucaggcuaugcugcuacgaugcaccgcgagggauucuugugcugcaaagugacagacacauugaacggggagagggucucuuuucccgugugcacguaugugccagcuacauugugugaccaaaugacuggcauacuggcaacagaugucagugcggacgacgcgcaaaaacugcugguugggcucaaccagcguauagucgucaacggucgcacccagagaaacaccaauaccaugaaaaauuaccuuuugcccguaguggcccaggcauuugcuaggugggcaaaggaauauaaggaagaucaagaagaugaaaggccacuaggacuacgagauagacaguuagucaugggguguuguugggcuuuuagaaggcacaagauaacaucuauuuauaagcgcccggauacccaaaccaucaucaaagugaacagcgauuuccacucauucgugcugcccaggauaggcaguaacacauuggagaucgggcugagaacaagaaucaggaaaauguuagaggagcacaaggagccgucaccucucauuaccgccgaggacguacaagaagcuaagugcgcagccgaugaggcuaaggaggugcgugaagccgaggaguugcgcgcagcucuaccaccuuuggcagcugauguugaggagcccacucuggaagccgaugucgacuugauguuacaagaggcuggggcc

SEQ ID NO: 218 - protein, 794 aa, Unknown
  Non-structural protein 2
GSVETPRGLIKVTSYDGEDKIGSYAVLSPQAVLKSEKLSCIHPLAEQVIVITHSGRKGRYAVEPYHGKVVVPEGHAIPVQDFQALSESATIVYNEREFVNRYLHHIATHGGALNTDEEYYKTVKPSEHDGEYLYDIDRKQCVKKELVTGLGLTGELVDPPFHEFAYESLRTRPAAPYQVPTIGVYGVPGSGKSGIIKSAVTKKDLVVSAKKENCAEIIRDVKKMKGLDVNARTVDSVLLNGCKHPVETLYIDEAFACHAGTLRALIAIIRPKKAVLCGDPKQCGFFNMMCLKVHFNHEICTQVFHKSISRRCTKSVTSVVSTLFYDKKMRTTNPKETKIVIDTTGSTKPKQDDLILTCFRGWVKQLQIDYKGNEIMTAAASQGLTRKGVYAVRYKVNENPLYAPTSEHVNVLLTRTEDRIVWKTLAGDPWIKTLTAKYPGNFTATIEEWQAEHDAIMRHILERPDPTDVFQNKANVCWAKALVPVLKTAGIDMTTEQWNTVDYFETDKAHSAEIVLNQLCVRFFGLDLDSGLFSAPTVPLSIRNNHWDNSPSPNMYGLNKEVVRQLSRRYPQLPRAVATGRVYDMNTGTLRNYDPRINLVPVNRRLPHALVLHHNEHPQSDFSSFVSKLKGRTVLVVGEKLSVPGKMVDWLSDRPEATFRARLDLGIPGDVPKYDIIFVNVRTPYKYHHYQQCEDHAIKLSMLTKKACLHLNPGGTCVSIGYGYADRASESIIGAIARQFKFSRVCKPKSSLEETEVLFVFIGYDRKARTHNSYKLSSTLTNIYTGSRLHEAGC

SEQ ID NO: 219 - DNA, 2382 bp, Unknown
  Non-structural protein 2
ggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaattcttacaagctttcatcaaccttgaccaacatttatacaggttccagactccacgaagccggatgt

SEQ ID NO: 220 - RNA, 2382 nt, Unknown
  Non-structural protein 2
ggcucaguggagacaccucguggcuugauaaagguuaccagcuacgauggcgaggacaagaucggcucuuacgcugugcuuucuccgcaggcuguacucaagagugaaaaauuaucuugcauccacccucucgcugaacaagucauagugauaacacacucuggccgaaaagggcguuaugccguggaaccauaccaugguaaaguaguggugccagagggacaugcaauacccguccaggacuuucaagcucugagugaaagugccaccauuguguacaacgaacgugaguucguaaacagguaccugcaccauauugccacacauggaggagcgcugaacacugaugaagaauauuacaaaacugucaagcccagcgagcacgacggcgaauaccuguacgacaucgacaggaaacagugcgucaagaaagaacuagucacugggcuagggcucacaggcgagcugguggauccucccuuccaugaauucgccuacgagagucugagaacacgaccagccgcuccuuaccaaguaccaaccauagggguguauggcgugccaggaucaggcaagucuggcaucauuaaaagcgcagucaccaaaaaagaucuaguggugagcgccaagaaagaaaacugugcagaaauuauaagggacgucaagaaaaugaaagggcuggacgucaaugccagaacuguggacucagugcucuugaauggaugcaaacaccccguagagacccuguauauugacgaagcuuuugcuugucaugcagguacucucagagcgcucauagccauuauaagaccuaaaaaggcagugcucugcggggaucccaaacagugcgguuuuuuuaacaugaugugccugaaagugcauuuuaaccacgagauuugcacacaagucuuccacaaaagcaucucucgccguugcacuaaaucugugacuucggucgucucaaccuuguuuuacgacaaaaaaaugagaacgacgaauccgaaagagacuaagauugugauugacacuaccggcaguaccaaaccuaagcaggacgaucucauucucacuuguuucagagggugggugaagcaguugcaaauagauuacaaaggcaacgaaauaaugacggcagcugccucucaagggcugacccguaaagguguguaugccguucgguacaaggugaaugaaaauccucuguacgcacccaccucagaacaugugaacguccuacugacccgcacggaggaccgcaucguguggaaaacacuagccggcgacccauggauaaaaacacugacugccaaguacccugggaauuucacugccacgauagaggaguggcaagcagagcaugaugccaucaugaggcacaucuuggagagaccggacccuaccgacgucuuccagaauaaggcaaacguguguugggccaaggcuuuagugccggugcugaagaccgcuggcauagacaugaccacugaacaauggaacacuguggauuauuuugaaacggacaaagcucacucagcagagauaguauugaaccaacuaugcgugagguucuuuggacucgaucuggacuccggucuauuuucugcacccacuguuccguuauccauuaggaauaaucacugggauaacuccccgucgccuaacauguacgggcugaauaaagaagugguccgucagcucucucgcagguacccacaacugccucgggcaguugccacuggaagagucuaugacaugaacacugguacacugcgcaauuaugauccgcgcauaaaccuaguaccuguaaacagaagacugccucaugcuuuaguccuccaccauaaugaacacccacagagugacuuuucuucauucgucagcaaauugaagggcagaacuguccugguggucggggaaaaguuguccgucccaggcaaaaugguugacugguugucagaccggccugaggcuaccuucagagcucggcuggauuuaggcaucccaggugaugugcccaaauaugacauaauauuuguuaaugugaggaccccauauaaauaccaucacuaucagcagugugaagaccaugccauuaagcuuagcauguugaccaagaaagcuugucugcaucugaaucccggcggaaccugugucagcauagguuaugguuacgcugacagggccagcgaaagcaucauuggugcuauagcgcggcaguucaaguuuucccggguaugcaaaccgaaauccucacuugaagagacggaaguucuguuuguauucauuggguacgaucgcaaggcccguacgcacaauucuuacaagcuuucaucaaccuugaccaacauuuauacagguuccagacuccacgaagccggaugu

SEQ ID NO: 221 - protein, 556 aa, Unknown
  Non-structural protein 3
APSYHVVRGDIATATEGVIINAANSKGQPGGGVCGALYKKFPESFDLQPIEVGKARLVKGAAKHIIHAVGPNFNKVSEVEGDKQLAEAYESIAKIVNDNNYKSVAIPLLSTGIFSGNKDRLTQSLNHLLTALDTTDADVAIYCRDKKWEMTLKEAVARREAVEEICISDDSSVTEPDAELVRVHPKSSLAGRKGYSTSDGKTFSYLEGTKFHQAAKDIAEINAMWPVATEANEQVCMYILGESMSSIRSKCPVEESEASTPPSTLPCLCIHAMTPERVQRLKASRPEQITVCSSFPLPKYRITGVQKIQCSQPILFSPKVPAYIHPRKYLVETPPVDETPEPSAENQSTEGTPEQPPLITEDETRTRTPEPIIIEEEEEDSISLLSDGPTHQVLQVEADIHGPPSVSSSSWSIPHASDFDVDSLSILDTLEGASVTSGATSAETNSYFAKSMEFLARPVPAPRTVFRNPPHPAPRTRTPSLAPSRACSRTSLVSTPPGVNRVITREELEALTPSRTPSRSVSRTSLVSNPPGVNRVITREEFEAFVAQQQRFDAGA

SEQ ID NO: 222 - DNA, 1671 bp, Unknown
  Non-structural protein 3
gcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgca

SEQ ID NO: 223 - RNA, 1671 nt, Unknown
  Non-structural protein 3
gcacccucauaucauguggugcgaggggauauugccacggccaccgaaggagugauuauaaaugcugcuaacagcaaaggacaaccuggcggaggggugugcggagcgcuguauaagaaauucccggaaagcuucgauuuacagccgaucgaaguaggaaaagcgcgacuggucaaaggugcagcuaaacauaucauucaugccguaggaccaaacuucaacaaaguuucggagguugaaggugacaaacaguuggcagaggcuuaugaguccaucgcuaagauugucaacgauaacaauuacaagucaguagcgauuccacuguuguccaccggcaucuuuuccgggaacaaagaucgacuaacccaaucauugaaccauuugcugacagcuuuagacaccacugaugcagauguagccauauacugcagggacaagaaaugggaaaugacucucaaggaagcaguggcuaggagagaagcaguggaggagauaugcauauccgacgacucuucagugacagaaccugaugcagagcuggugagggugcauccgaagaguucuuuggcuggaaggaagggcuacagcacaagcgauggcaaaacuuucucauauuuggaagggaccaaguuucaccaggcggccaaggauauagcagaaauuaaugccauguggcccguugcaacggaggccaaugagcagguaugcauguauauccucggagaaagcaugagcaguauuaggucgaaaugccccgucgaagagucggaagccuccacaccaccuagcacgcugccuugcuugugcauccaugccaugacuccagaaagaguacagcgccuaaaagccucacguccagaacaaauuacugugugcucauccuuuccauugccgaaguauagaaucacuggugugcagaagauccaaugcucccagccuauauuguucucaccgaaagugccugcguauauucauccaaggaaguaucucguggaaacaccaccgguagacgagacuccggagccaucggcagagaaccaauccacagaggggacaccugaacaaccaccacuuauaaccgaggaugagaccaggacuagaacgccugagccgaucaucaucgaagaggaagaagaggauagcauaaguuugcugucagauggcccgacccaccaggugcugcaagucgaggcagacauucacgggccgcccucuguaucuagcucauccugguccauuccucaugcauccgacuuugauguggacaguuuauccauacuugacacccuggagggagcuagcgugaccagcggggcaacgucagccgagacuaacucuuacuucgcaaagaguauggaguuucuggcgcgaccggugccugcgccucgaacaguauucaggaacccuccacaucccgcuccgcgcacaagaacaccgucacuugcacccagcagggccugcucgagaaccagccuaguuuccaccccgccaggcgugaauagggugaucacuagagaggagcucgaggcgcuuaccccgucacgcacuccuagcaggucggucucgagaaccagccuggucuccaacccgccaggcguaaauagggugauuacaagagaggaguuugaggcguucguagcacaacaacaaugacgguuugaugcgggugca

SEQ ID NO: 224 - protein, 607 aa, Unknown
  Non-structural protein 4
YIFSSDTGQGHLQQKSVRQTVLSEVVLERTELEISYAPRLDQEKEELLRKKLQLNPTPANRSRYQSRKVENMKAITARRILQGLGHYLKAEGKVECYRTLHPVPLYSSSVNRAFSSPKVAVEACNAMLKENFPTVASYCIIPEYDAYLDMVDGASCCLDTASFCPAKLRSFPKKHSYLEPTIRSAVPSAIQNTLQNVLAAATKRNCNVTQMRELPVLDSAAFNVECFKKYACNNEYWETFKENPIRLTEENVVNYITKLKGPKAAALFAKTHNLNMLQDIPMDRFVMDLKRDVKVTPGTKHTEERPKVQVIQAADPLATAYLCGIHRELVRRLNAVLLPNIHTLFDMSAEDFDAIIAEHFQPGDCVLETDIASFDKSEDDAMALTALMILEDLGVDAELLTLIEAAFGEISSIHLPTKTKFKFGAMMKSGMFLTLFVNTVINIVIASRVLRERLTGSPCAAFIGDDNIVKGVKSDKLMADRCATWLNMEVKIIDAVVGEKAPYFCGGFILCDSVTGTACRVADPLKRLFKLGKPLAADDEHDDDRRRALHEESTRWNRVGILSELCKAVESRYETVGTSIIVMAMTTLASSVKSFSYLRGAPITLYG

SEQ ID NO: 225 - DNA, 1821 bp, Unknown
  Non-structural protein 4
tacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggc

SEQ ID NO: 226 - RNA, 1821 nt, Unknown
  Non-structural protein 4
uacaucuuuuccuccgacaccggucaagggcauuuacaacaaaaaucaguaaggcaaacggugcuauccgaagugguguuggagaggaccgaauuggagauuucguaugccccgcgccucgaccaagaaaaagaagaauuacuacgcaagaaauuacaguuaaaucccacaccugcuaacagaagcagauaccaguccaggaagguggagaacaugaaagccauaacagcuagacguauucugcaaggccuagggcauuauuugaaggcagaaggaaaaguggagugcuaccgaacccugcauccuguuccuuuguauucaucuagugugaaccgugccuuuucaagccccaaggucgcaguggaagccuguaacgccauguugaaagagaacuuuccgacuguggcuucuuacuguauuauuccagaguacgaugccuauuuggacaugguugacggagcuucaugcugcuuagacacugccaguuuuugcccugcaaagcugcgcagcuuuccaaagaaacacuccuauuuggaacccacaauacgaucggcagugccuucagcgauccagaacacgcuccagaacguccuggcagcugccacaaaaagaaauugcaaugucacgcaaaugagagaauugcccguauuggauucggcggccuuuaauguggaaugcuucaagaaauaugcguguaauaaugaauauugggaaacguuuaaagaaaaccccaucaggcuuacugaagaaaacgugguaaauuacauuaccaaauuaaaaggaccaaaagcugcugcucuuuuugcgaagacacauaauuugaauauguugcaggacauaccaauggacagguuuguaauggacuuaaagagagacgugaaagugacuccaggaacaaaacauacugaagaacggcccaagguacaggugauccaggcugccgauccgcuagcaacagcguaucugugcggaauccaccgagagcugguuaggagauuaaaugcgguccugcuuccgaacauucauacacuguuugauaugucggcugaagacuuugacgcuauuauagccgagcacuuccagccuggggauuguguucuggaaacugacaucgcgucguuugauaaaagugaggacgacgccauggcucugaccgcguuaaugauucuggaagacuuagguguggacgcagagcuguugacgcugauugaggcggcuuucggcgaaauuucaucaauacauuugcccacuaaaacuaaauuuaaauucggagccaugaugaaaucuggaauguuccucacacuguuugugaacacagucauuaacauuguaaucgcaagcagaguguugagagaacggcuaaccggaucaccaugugcagcauucauuggagaugacaauaucgugaaaggagucaaaucggacaaauuaauggcagacaggugcgccaccugguugaauauggaagucaagauuauagaugcuguggugggcgagaaagcgccuuauuucuguggaggguuuauuuugugugacuccgugaccggcacagcgugccguguggcagacccccuaaaaaggcuguuuaagcuuggcaaaccucuggcagcagacgaugaacaugaugaugacaggagaagggcauugcaugaagagucaacacgcuggaaccgaguggguauucuuucagagcugugcaaggcaguagaaucaagguaugaaaccguaggaacuuccaucauaguuauggccaugacuacucuagcuagcaguguuaaaucauucagcuaccugagaggggccccuauaacucucuacggc

SEQ ID NO: 227 - RNA, 44 nt, Artificial Sequence
  5′UTR
augggcggcgcaugagagaagcccagaccaauuaccuacccaaa

SEQ ID NO: 228 - RNA, 142 nt, Artificial Sequence
  3′UTR
aauuggcaagcugcuuacauagaacucgcggcgauuggcaugccgccuuaaaauuuuuauuuuauuuuucuuuucuuuuccgaaucggauuuuguuuuuaauauuucaaaaaaaaaaaaaaaaaaaaaaaaaaaaaaaaaaa

SEQ ID NO: 229 - RNA, 7587 nt, Artificial Sequence
  RNA construct
uaauacgacucacuauagaugggcggcgcaugagagaagcccagaccaauuaccuacccaaaauggagaaaguucacguugacaucgaggaagacagcccauuccucagagcuuugcagcggagcuucccgcaguuugagguagaagccaagcaggucacugauaaugaccaugcuaaugccagagcguuuucgcaucuggcuucaaaacugaucgaaacggagguggacccauccgacacgauccuugacauuggaagugcgcccgcccgcagaauguauucuaagcacaaguaucauuguaucuguccgaugagaugugcggaagauccggacagauuguauaaguaugcaacuaagcugaagaaaaacuguaaggaaauaacugauaaggaauuggacaagaaaaugaaggagcuggccgccgucaugagcgacccugaccuggaaacugagacuaugugccuccacgacgacgagucgugucgcuacgaagggcaagucgcuguuuaccaggauguauacgcgguugacggaccgacaagucucuaucaccaagccaauaagggaguuagagucgccuacuggauaggcuuugacaccaccccuuuuauguuuaagaacuuggcuggagcauauccaucauacucuaccaacugggccgacgaaaccguguuaacggcucguaacauaggccuaugcagcucugacguuauggagcggucacguagagggauguccauucuuagaaagaaguauuugaaaccauccaacaauguucuauucucuguuggcucgaccaucuaccacgagaagagggacuuacugaggagcuggcaccugccgucuguauuucacuuacguggcaagcaaaauuacacaugucggugugagacuauaguuaguugcgacggguacgucguuaaaagaauagcuaucaguccaggccuguaugggaagccuucaggcuaugcugcuacgaugcaccgcgagggauucuugugcugcaaagugacagacacauugaacggggagagggucucuuuucccgugugcacguaugugccagcuacauugugugaccaaaugacuggcauacuggcaacagaugucagugcggacgacgcgcaaaaacugcugguugggcucaaccagcguauagucgucaacggucgcacccagagaaacaccaauaccaugaaaaauuaccuuuugcccguaguggcccaggcauuugcuaggugggcaaaggaauauaaggaagaucaagaagaugaaaggccacuaggacuacgagauagacaguuagucaugggguguuguugggcuuuuagaaggcacaagauaacaucuauuuauaagcgcccggauacccaaaccaucaucaaagugaacagcgauuuccacucauucgugcugcccaggauaggcaguaacacauuggagaucgggcugagaacaagaaucaggaaaauguuagaggagcacaaggagccgucaccucucauuaccgccgaggacguacaagaagcuaagugcgcagccgaugaggcuaaggaggugcgugaagccgaggaguugcgcgcagcucuaccaccuuuggcagcugauguugaggagcccacucuggaggcagacgucgacuugauguuacaagaggcuggggccggcucaguggagacaccucguggcuugauaaagguuaccagcuacgauggcgaggacaagaucggcucuuacgcugugcuuucuccgcaggcuguacucaagagugaaaaauuaucuugcauccacccucucgcugaacaagucauagugauaacacacucuggccgaaaagggcguuaugccguggaaccauaccaugguaaaguaguggugccagagggacaugcaauacccguccaggacuuucaagcucugagugaaagugccaccauuguguacaacgaacgugaguucguaaacagguaccugcaccauauugccacacauggaggagcgcugaacacugaugaagaauauuacaaaacugucaagcccagcgagcacgacggcgaauaccuguacgacaucgacaggaaacagugcgucaagaaagaacuagucacugggcuagggcucacaggcgagcugguggauccucccuuccaugaauucgccuacgagagucugagaacacgaccagccgcuccuuaccaaguaccaaccauagggguguauggcgugccaggaucaggcaagucuggcaucauuaaaagcgcagucaccaaaaaagaucuaguggugagcgccaagaaagaaaacugugcagaaauuauaagggacgucaagaaaaugaaagggcuggacgucaaugccagaacuguggacucagugcucuugaauggaugcaaacaccccguagagacccuguauauugacgaagcuuuugcuugucaugcagguacucucagagcgcucauagccauuauaagaccuaaaaaggcagugcucugcggggaucccaaacagugcgguuuuuuuaacaugaugugccugaaagugcauuuuaaccacgagauuugcacacaagucuuccacaaaagcaucucucgccguugcacuaaaucugugacuucggucgucucaaccuuguuuuacgacaaaaaaaugagaacgacgaauccgaaagagacuaagauugugauugacacuaccggcaguaccaaaccuaagcaggacgaucucauucucacuuguuucagagggugggugaagcaguugcaaauagauuacaaaggcaacgaaauaaugacggcagcugccucucaagggcugacccguaaagguguguaugccguucgguacaaggugaaugaaaauccucuguacgcacccaccucagaacaugugaacguccuacugacccgcacggaggaccgcaucguguggaaaacacuagccggcgacccauggauaaaaacacugacugccaaguacccugggaauuucacugccacgauagaggaguggcaagcagagcaugaugccaucaugaggcacaucuuggagagaccggacccuaccgacgucuuccagaauaaggcaaacguguguugggccaaggcuuuagugccggugcugaagaccgcuggcauagacaugaccacugaacaauggaacacuguggauuauuuugaaacggacaaagcucacucagcagagauaguauugaaccaacuaugcgugagguucuuuggacucgaucuggacuccggucuauuuucugcacccacuguuccguuauccauuaggaauaaucacugggauaacuccccgucgccuaacauguacgggcugaauaaagaagugguccgucagcucucucgcagguacccacaacugccucgggcaguugccacuggaagagucuaugacaugaacacugguacacugcgcaauuaugauccgcgcauaaaccuaguaccuguaaacagaagacugccucaugcuuuaguccuccaccauaaugaacacccacagagugacuuuucuucauucgucagcaaauugaagggcagaacuguccugguggucggggaaaaguuguccgucccaggcaaaaugguugacugguugucagaccggccugaggcuaccuucagagcucggcuggauuuaggcaucccaggugaugugcccaaauaugacauaauauuuguuaaugugaggaccccauauaaauaccaucacuaucagcagugugaagaccaugccauuaagcuuagcauguugaccaagaaagcuugucugcaucugaaucccggcggaaccugugucagcauagguuaugguuacgcugacagggccagcgaaagcaucauuggugcuauagcgcggcaguucaaguuuucccggguaugcaaaccgaaauccucacuugaagagacggaaguucuguuuguauucauuggguacgaucgcaaggcccguacgcacaauucuuacaagcuuucaucaaccuugaccaacauuuauacagguuccagacuccacgaagccggaugugcacccucauaucauguggugcgaggggauauugccacggccaccgaaggagugauuauaaaugcugcuaacagcaaaggacaaccuggcggaggggugugcggagcgcuguauaagaaauucccggaaagcuucgauuuacagccgaucgaaguaggaaaagcgcgacuggucaaaggugcagcuaaacauaucauucaugccguaggaccaaacuucaacaaaguuucggagguugaaggugacaaacaguuggcagaggcuuaugaguccaucgcuaagauugucaacgauaacaauuacaagucaguagcgauuccacuguuguccaccggcaucuuuuccgggaacaaagaucgacuaacccaaucauugaaccauuugcugacagcuuuagacaccacugaugcagauguagccauauacugcagggacaagaaaugggaaaugacucucaaggaagcaguggcuaggagagaagcaguggaggagauaugcauauccgacgacucuucagugacagaaccugaugcagagcuggugagggugcauccgaagaguucuuuggcuggaaggaagggcuacagcacaagcgauggcaaaacuuucucauauuuggaagggaccaaguuucaccaggcggccaaggauauagcagaaauuaaugccauguggcccguugcaacggaggccaaugagcagguaugcauguauauccucggagaaagcaugagcaguauuaggucgaaaugccccgucgaagagucggaagccuccacaccaccuagcacgcugccuugcuugugcauccaugccaugacuccagaaagaguacagcgccuaaaagccucacguccagaacaaauuacugugugcucauccuuuccauugccgaaguauagaaucacuggugugcagaagauccaaugcucccagccuauauuguucucaccgaaagugccugcguauauucauccaaggaaguaucucguggaaacaccaccgguagacgagacuccggagccaucggcagagaaccaauccacagaggggacaccugaacaaccaccacuuauaaccgaggaugagaccaggacuagaacgccugagccgaucaucaucgaagaggaagaagaggauagcauaaguuugcugucagauggcccgacccaccaggugcugcaagucgaggcagacauucacgggccgcccucuguaucuagcucauccugguccauuccucaugcauccgacuuugauguggacaguuuauccauacuugacacccuggagggagcuagcgugaccagcggggcaacgucagccgagacuaacucuuacuucgcaaagaguauggaguuucuggcgcgaccggugccugcgccucgaacaguauucaggaacccuccacaucccgcuccgcgcacaagaacaccgucacuugcacccagcagggccugcucgagaaccagccuaguuuccaccccgccaggcgugaauagggugaucacuagagaggagcucgaggcgcuuaccccgucacgcacuccuagcaggucggucucgagaaccagccuggucuccaacccgccaggcguaaauagggugauuacaagagaggaguuugaggcguucguagcacaacaacaaugacgguuugaugcgggugcauacaucuuuuccuccgacaccggucaagggcauuuacaacaaaaaucaguaaggcaaacggugcuauccgaagugguguuggagaggaccgaauuggagauuucguaugccccgcgccucgaccaagaaaaagaagaauuacuacgcaagaaauuacaguuaaaucccacaccugcuaacagaagcagauaccaguccaggaagguggagaacaugaaagccauaacagcuagacguauucugcaaggccuagggcauuauuugaaggcagaaggaaaaguggagugcuaccgaacccugcauccuguuccuuuguauucaucuagugugaaccgugccuuuucaagccccaaggucgcaguggaagccuguaacgccauguugaaagagaacuuuccgacuguggcuucuuacuguauuauuccagaguacgaugccuauuuggacaugguugacggagcuucaugcugcuuagacacugccaguuuuugcccugcaaagcugcgcagcuuuccaaagaaacacuccuauuuggaacccacaauacgaucggcagugccuucagcgauccagaacacgcuccagaacguccuggcagcugccacaaaaagaaauugcaaugucacgcaaaugagagaauugcccguauuggauucggcggccuuuaauguggaaugcuucaagaaauaugcguguaauaaugaauauugggaaacguuuaaagaaaaccccaucaggcuuacugaagaaaacgugguaaauuacauuaccaaauuaaaaggaccaaaagcugcugcucuuuuugcgaagacacauaauuugaauauguugcaggacauaccaauggacagguuuguaauggacuuaaagagagacgugaaagugacuccaggaacaaaacauacugaagaacggcccaagguacaggugauccaggcugccgauccgcuagcaacagcguaucugugcggaauccaccgagagcugguuaggagauuaaaugcgguccugcuuccgaacauucauacacuguuugauaugucggcugaagacuuugacgcuauuauagccgagcacuuccagccuggggauuguguucuggaaacugacaucgcgucguuugauaaaagugaggacgacgccauggcucugaccgcguuaaugauucuggaagacuuagguguggacgcagagcuguugacgcugauugaggcggcuuucggcgaaauuucaucaauacauuugcccacuaaaacuaaauuuaaauucggagccaugaugaaaucuggaauguuccucacacuguuugugaacacagucauuaacauuguaaucgcaagcagaguguugagagaacggcuaaccggaucaccaugugcagcauucauuggagaugacaauaucgugaaaggagucaaaucggacaaauuaauggcagacaggugcgccaccugguugaauauggaagucaagauuauagaugcuguggugggcgagaaagcgccuuauuucuguggaggguuuauuuugugugacuccgugaccggcacagcgugccguguggcagacccccuaaaaaggcuguuuaagcuuggcaaaccucuggcagcagacgaugaacaugaugaugacaggagaagggcauugcaugaagagucaacacgcuggaaccgaguggguauucuuucagagcugugcaaggcaguagaaucaagguaugaaaccguaggaacuuccaucauaguuauggccaugacuacucuagcuagcaguguuaaaucauucagcuaccugagaggggccccuauaacucucuacggcuaaccugaauggacuacgacauagucuaguccgccaagucuagcau

SEQ ID NO: 230 - DNA, 7587 bp, Artificial Sequence
  Nucleic acid sequence encoding RNA construct
taatacgactcactatagatgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaattcttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagtctagcat

SEQ ID NO: 231 - DNA, 7587 bp, Artificial Sequence; identical to SEQ ID NO: 230
  Recombinant vector

SEQ ID NO: 232 - protein, 113 aa, Homo sapiens
MPVERMRMRPWLEEQINSNTIPGLKWLNKEKKIFQIPWMHAARHGWDVEKDAPLFRNWAIHTGKHQPGVDKPDPKTWKANFRCAMNSLPDIEEVKDKSIKKGNNAFRVYRMLP

SEQ ID NO: 233 - DNA, 339 bp, Homo sapiens
atgccggtggaaaggatgcgcatgcgcccgtggctggaggagcagataaactccaacacgatcccggggctcaagtggcttaacaaggaaaagaagatttttcagatcccctggatgcatgcggctagacatgggtgggatgtggaaaaagatgcaccactctttagaaactgggcaatccatacaggaaagcatcaaccaggagtagataaacctgatcccaaaacatggaaggcgaatttcagatgcgccatgaattccttgcctgatattgaagaagtcaaggataaaagcataaagaaaggaaataatgccttcagggtctaccgaatgctgccc

SEQ ID NO: 234 - RNA, 339 nt, Homo sapiens
augccgguggaaaggaugcgcaugcgcccguggcuggaggagcagauaaacuccaacacgaucccggggcucaaguggcuuaacaaggaaaagaagauuuuucagauccccuggaugcaugcggcuagacaugggugggauguggaaaaagaugcaccacucuuuagaaacugggcaauccauacaggaaagcaucaaccaggaguagauaaaccugaucccaaaacauggaaggcgaauuucagaugcgccaugaauuccuugccugauauugaagaagucaaggauaaaagcauaaagaaaggaaauaaugccuucagggucuaccgaaugcugccc

SEQ ID NO: 235 - DNA, 342 bp, Artificial Sequence
  Codon optimised DNA encoding IRF2
atgcccgtggaacggatgagaatgaggccctggctggaagaacagatcaacagcaacacaatccccggcctgaagtggctgaacaaagagaagaagatctttcagatcccctggatgcacgccgccagacacggatgggatgtcgagaaagatgcccctctgttcagaaactgggccatccacaccggcaaacaccagcctggcgtggacaagcctgatcctaagacctggaaggccaacttcagatgcgccatgaacagcctgcctgacatcgaggaagtgaaggacaagagcatcaagaagggcaacaacgccttccgggtgtacagaatgctgccctga

SEQ ID NO: 236 - RNA, 342 nt, Artificial Sequence
  Codon optimised RNA encoding IRF2
augcccguggaacggaugagaaugaggcccuggcuggaagaacagaucaacagcaacacaauccccggccugaaguggcugaacaaagagaagaagaucuuucagauccccuggaugcacgccgccagacacggaugggaugucgagaaagaugccccucuguucagaaacugggccauccacaccggcaaacaccagccuggcguggacaagccugauccuaagaccuggaaggccaacuucagaugcgccaugaacagccugccugacaucgaggaagugaaggacaagagcaucaagaagggcaacaacgccuuccggguguacagaaugcugcccuga

SEQ ID NO: 237 - protein, 115 aa, Homo sapiens
MALHPRRVRLKPWLVAQVDSGLYPGLIWLHRDSKRFQIPWKHATRHSPQQEEENTIFKAWAVETGKYQEGVDDPDPAKWKAQLRCALNKSREFNLMYDGTKEVPMNPVKIYQVCD

SEQ ID NO: 238 - DNA, 345 bp, Homo sapiens
atggccctccacccccgcagagtccggctaaagccctggctggtggcccaggtggatagtggcctctaccctgggctcatctggctacacagggactctaaacgcttccagattccctggaaacatgccacccggcatagccctcaacaagaagaggaaaataccatttttaaggcctgggctgtagagacagggaagtaccaggaaggggtggatgaccctgacccagctaaatggaaggcccagctgcgctgtgctctcaataagagcagagaattcaacctgatgtatgatggcaccaaggaggtgcccatgaacccagtgaagatatatcaagtgtgtgac

SEQ ID NO: 239 - RNA, 345 nt, Homo sapiens
auggcccuccacccccgcagaguccggcuaaagcccuggcugguggcccagguggauaguggccucuacccugggcucaucuggcuacacagggacucuaaacgcuuccagauucccuggaaacaugccacccggcauagcccucaacaagaagaggaaaauaccauuuuuaaggccugggcuguagagacagggaaguaccaggaagggguggaugacccugacccagcuaaauggaaggcccagcugcgcugugcucucaauaagagcagagaauucaaccugauguaugauggcaccaaggaggugcccaugaacccagugaagauauaucaagugugugac

SEQ ID NO: 240 - DNA, 348 bp, Artificial Sequence
  Codon optimised DNA encoding IRF6
atggccctgcatcctagaagagtgcggctgaagccttggctggtggctcaagtggatagcggcctgtatcctggcctgatctggctgcacagagacagcaagcggtttcagatcccctggaagcacgccaccagacacagccctcagcaagaggaagagaacaccatcttcaaggcctgggccgtcgagacaggcaagtaccaagaaggcgtggacgaccccgatcctgccaaatggaaagcccagctgagatgcgccctgaacaagagccgcgagttcaacctgatgtacgacggcaccaaagaggtgcccatgaatcccgtgaagatctaccaagtgtgcgactga

SEQ ID NO: 241 - RNA, 348 nt, Artificial Sequence
  Codon optimised RNA encoding IRF6
auggcccugcauccuagaagagugcggcugaagccuuggcugguggcucaaguggauagcggccuguauccuggccugaucuggcugcacagagacagcaagcgguuucagauccccuggaagcacgccaccagacacagcccucagcaagaggaagagaacaccaucuucaaggccugggccgucgagacaggcaaguaccaagaaggcguggacgaccccgauccugccaaauggaaagcccagcugagaugcgcccugaacaagagccgcgaguucaaccugauguacgacggcaccaaagaggugcccaugaaucccgugaagaucuaccaagugugcgacuga

SEQ ID NO: 242 - protein, 229 aa, Homo sapiens
EAVRLIMDSLHMAAREQQVYCEEMREERQDRLKFIDKQLELLAQDYKLRIKQITEEVERQVSTAMAEEIRRLSVLVDDYQMDFHPSPVVLKVYKNELHRHIEEGLGRNMSDRCSTAITNSLQTMQQDMIDGLKPLLPVSVRSQIDMLVPRQCFSLNYDLNCDKLCADFQEDIEFHFSLGWTMLVNRFLGPKNSRRALMGYNDQVQRPIPLTPANPSMPPLPQGSLTQEE

SEQ ID NO: 243 - DNA, 687 bp, Homo sapiens
gaggcggttcgactcatcatggactccctgcacatggcggctcgggagcagcaggtttactgcgaggaaatgcgtgaagagcggcaagaccgactgaaatttattgacaaacagctggagctcttggctcaagactataagctgcgaattaagcagattacggaggaagtggagaggcaggtgtcgactgcaatggccgaggagatcaggcgcctctctgtactggtggacgattaccagatggacttccacccttctccagtagtcctcaaggtttataagaatgagctgcaccgccacatagaggaaggactgggtcgaaacatgtctgaccgctgctccacggccatcaccaactccctgcagaccatgcagcaggacatgatagatggcttgaaacccctccttcctgtgtctgtgcggagtcagatagacatgctggtcccacgccagtgcttctccctcaactatgacctaaactgtgacaagctgtgtgctgacttccaggaagacattgagttccatttctctctcggatggaccatgctggtgaataggttcctgggccccaagaacagccgtcgggccttgatgggctacaatgaccaggtccagcgtcccatccctctgacgccagccaaccccagcatgcccccactgccacagggctcgctcacccaggaggag

SEQ ID NO: 244 - RNA, 687 nt, Homo sapiens
gaggcgguucgacucaucauggacucccugcacauggcggcucgggagcagcagguuuacugcgaggaaaugcgugaagagcggcaagaccgacugaaauuuauugacaaacagcuggagcucuuggcucaagacuauaagcugcgaauuaagcagauuacggaggaaguggagaggcaggugucgacugcaauggccgaggagaucaggcgccucucuguacugguggacgauuaccagauggacuuccacccuucuccaguaguccucaagguuuauaagaaugagcugcaccgccacauagaggaaggacugggucgaaacaugucugaccgcugcuccacggccaucaccaacucccugcagaccaugcagcaggacaugauagauggcuugaaaccccuccuuccugugucugugcggagucagauagacaugcuggucccacgccagugcuucucccucaacuaugaccuaaacugugacaagcugugugcugacuuccaggaagacauugaguuccauuucucucucggauggaccaugcuggugaauagguuccugggccccaagaacagccgucgggccuugaugggcuacaaugaccagguccagcgucccaucccucugacgccagccaaccccagcaugcccccacugccacagggcucgcucacccaggaggag

SEQ ID NO: 245 - DNA, 693 bp, Artificial Sequence
  Codon optimised DNA encoding MFN2
atggaggccgtcagactgatcatggacagcctgcatatggccgccagagagcagcaggtctactgcgaggaaatgcgggaagagagacaggaccggctgaagttcatcgacaagcagctggaactgctggcccaggactacaagctgcggatcaagcagatcaccgaagaggtggaaagacaggtgtccaccgccatggccgaggaaatcagacgactgagcgtgctggtggacgactaccagatggactttcacccctctccagtggtgctgaaggtgtacaagaacgagctgcaccggcacatcgaggaaggcctgggcagaaacatgagcgacagatgcagcaccgccatcaccaatagcctgcagaccatgcagcaggacatgatcgacggcctgaaacctctgctgcctgtgtccgtcagatcccagatcgacatgctggtgcccagacagtgcttcagcctgaactacgacctgaactgcgacaagctgtgcgccgacttccaagaggacatcgagttccacttcagcctcggctggacaatgctggtcaacagatttctgggccccaagaacagcagacgggccctgatgggctacaacgatcaggtgcagaggcccattcctctgacacccgccaatcctagcatgcctccactgcctcagggcagcctgacacaagaagaatga

SEQ ID NO: 246 - RNA, 693 nt, Artificial Sequence
  Codon optimised RNA encoding MFN2
auggaggccgucagacugaucauggacagccugcauauggccgccagagagcagcaggucuacugcgaggaaaugcgggaagagagacaggaccggcugaaguucaucgacaagcagcuggaacugcuggcccaggacuacaagcugcggaucaagcagaucaccgaagagguggaaagacagguguccaccgccauggccgaggaaaucagacgacugagcgugcugguggacgacuaccagauggacuuucaccccucuccaguggugcugaagguguacaagaacgagcugcaccggcacaucgaggaaggccugggcagaaacaugagcgacagaugcagcaccgccaucaccaauagccugcagaccaugcagcaggacaugaucgacggccugaaaccucugcugccuguguccgucagaucccagaucgacaugcuggugcccagacagugcuucagccugaacuacgaccugaacugcgacaagcugugcgccgacuuccaagaggacaucgaguuccacuucagccucggcuggacaaugcuggucaacagauuucugggccccaagaacagcagacgggcccugaugggcuacaacgaucaggugcagaggcccauuccucugacacccgccaauccuagcaugccuccacugccucagggcagccugacacaagaagaauga

SEQ ID NO: 247 - protein, 463 aa, Homo sapiens
GCELVDLADEVASVYQSYQPRTSDRPPDPLEPPSLPAERPGPPTPAAAHSIPYNSCREKEPSYPMPVQETQAPESPGENSEQALQTLSPRAIPRNPDGGPLESSSDLAALSPLTSSGHQEQDTELGSTHTAGATSSLTPSRGPVSPSVSFQPLARSTPRASRLPGPTGSVVSTGTSFSSSSPGLASAGAAEGKQGAESDQAEPIICSSGAEAPANSLPSKVPTTLMPVNTVALKVPANPASVSTVPSKLPTSSKPPGAVPSNALTNPAPSKLPINSTRAGMVPSKVPTSMVLTKVSASTVPTDGSSRNEETPAAPTPAGATGGSSAWLDSSSENRGLGSELSKPGVLASQVDSPFSGCFEDLAISASTSLGMGPCHGPEENEYKSEGTFGIHVAENPSIQLLEGNPGPPADPDGGPRPQADRKFQEREVPCHRPSPGALWLQVAVTGVLVVTLLVVLYRRRLH

SEQ ID NO: 248 - DNA, 1389 bp, Homo sapiens
ggctgtgagctagttgatctcgcggacgaagtggcctctgtctaccagagctaccagcctcggacctcggaccgtcccccagacccactggagccaccgtcacttcctgctgagaggccagggccccccacacctgctgcggcccacagcatcccctacaacagctgcagagagaaggagccaagttaccccatgcctgtccaggagacccaggcgccagagtccccaggagagaattcagagcaagccctgcagacgctcagccccagagccatcccaaggaatccagatggtggccccctggagtcctcctctgacctggcagccctcagccctctgacctccagcgggcatcaggagcaggacacagaactgggcagtacccacacagcaggtgcgacctccagcctcacaccatcccgtgggcctgtgtctccatctgtctccttccagcccctggcccgttccacccccagggcaagccgcttgcctggacccacagggtcagttgtatctactggcacctccttctcctcctcatcccctggcttggcctctgcaggggctgcagagggtaaacagggtgcagagagtgaccaggccgagcctatcatctgctccagtggggcagaggcacctgccaactctctgccctccaaagtgcctaccaccttgatgcctgtgaacacagtggccctgaaagtgcctgccaacccagcatctgtcagcacagtgccctccaagttgccaactagctcaaagccccctggtgcagtgccttctaatgcgctcaccaatccagcaccatccaaattgcccatcaactcaacccgtgctggcatggtgccatccaaagtgcctactagcatggtgctcaccaaggtgtctgccagcacagtccccactgacgggagcagcagaaatgaggagaccccagcagctccaacacccgccggcgccactggaggcagctcagcctggctagacagcagctctgagaataggggccttgggtcggagctgagtaagcctggcgtgctggcatcccaggtagacagcccgttctcgggctgcttcgaggatcttgccatcagtgccagcacctccttgggcatggggccctgccatggcccagaggagaatgagtataagtccgagggcacctttgggatccacgtggctgagaaccccagcatccagctcctggagggcaaccctgggccacctgcggacccggatggcggccccaggccacaagccgaccggaagttccaggagagggaggtgccatgccacaggccctcacctggggctctgtggctccaggtggctgtgacaggggtgctggtagtcacactcctggtggtgctgtaccggcggcgtctgcac

SEQ ID NO: 249 - RNA, 1389 nt, Homo sapiens
ggcugugagcuaguugaucucgcggacgaaguggccucugucuaccagagcuaccagccucggaccucggaccgucccccagacccacuggagccaccgucacuuccugcugagaggccagggccccccacaccugcugcggcccacagcauccccuacaacagcugcagagagaaggagccaaguuaccccaugccuguccaggagacccaggcgccagaguccccaggagagaauucagagcaagcccugcagacgcucagccccagagccaucccaaggaauccagaugguggcccccuggaguccuccucugaccuggcagcccucagcccucugaccuccagcgggcaucaggagcaggacacagaacugggcaguacccacacagcaggugcgaccuccagccucacaccaucccgugggccugugucuccaucugucuccuuccagccccuggcccguuccacccccagggcaagccgcuugccuggacccacagggucaguuguaucuacuggcaccuccuucuccuccucauccccuggcuuggccucugcaggggcugcagaggguaaacagggugcagagagugaccaggccgagccuaucaucugcuccaguggggcagaggcaccugccaacucucugcccuccaaagugccuaccaccuugaugccugugaacacaguggcccugaaagugccugccaacccagcaucugucagcacagugcccuccaaguugccaacuagcucaaagcccccuggugcagugccuucuaaugcgcucaccaauccagcaccauccaaauugcccaucaacucaacccgugcuggcauggugccauccaaagugccuacuagcauggugcucaccaaggugucugccagcacaguccccacugacgggagcagcagaaaugaggagaccccagcagcuccaacacccgccggcgccacuggaggcagcucagccuggcuagacagcagcucugagaauaggggccuugggucggagcugaguaagccuggcgugcuggcaucccagguagacagcccguucucgggcugcuucgaggaucuugccaucagugccagcaccuccuugggcauggggcccugccauggcccagaggagaaugaguauaaguccgagggcaccuuugggauccacguggcugagaaccccagcauccagcuccuggagggcaacccugggccaccugcggacccggauggcggccccaggccacaagccgaccggaaguuccaggagagggaggugccaugccacaggcccucaccuggggcucuguggcuccagguggcugugacaggggugcugguagucacacuccugguggugcuguaccggcggcgucugcac

SEQ ID NO: 250 - DNA, 1395 bp, Artificial Sequence
  Codon optimised DNA encoding MAVS
atgggctgtgaactggtggatctggccgatgaagtggccagcgtgtaccagagctaccagcctagaaccagcgaccggcctcctgatcctctggaacctccatctctgcccgccgaaagacctggacctcctacaccagctgccgctcacagcatcccttacaacagctgcagagagaaagaacctagctaccccatgcctgtgcaagagacacaggccccagaaagccctggcgagaattctgaacaggccctgcagacactgagccccagagccattcctagaaaccctgatggcggccctctggaaagcagcagtgatctggctgctctgagccctctgacaagctctggacaccaagagcaggataccgagctgggcagcacacatacagccggcgctacaagcagcctgacaccttctagaggccccgtgtctcccagcgtgtcatttcagcctctggccaggtctacccctagagcctctagactgcctggacctacaggcagcgtggtgtctaccggcacaagcttcagctctagctctcctggactggcctctgctggtgccgctgagggaaaacaaggcgccgaatctgatcaggccgagcctatcatctgtagcagcggagcagaagcccctgccaatagcctgcctagcaaggtgccaaccacactgatgcccgtgaacacagtggccctgaaggtgccagctaatcctgcctccgtgtccaccgtgccttctaagctgccaaccagctctaagccacctggcgccgtgccatctaacgccctgacaaatcctgctccaagcaagctgcccatcaacagcacaagagccggcatggtgccctctaaggtgcccacatctatggtgctgaccaaggtgtccgccagcaccgtgccaacagatggcagcagcagaaacgaggaaacccctgccgctcctactcctgctggcgctacaggcggatcttctgcctggctggatagcagctccgagaatagaggcctgggcagcgagctgtctaaacctggcgttctggcaagccaggtggacagccctttcagcggctgctttgaggacctggctatcagcgcctctacaagcctcggcatgggaccttgtcacggccccgaggaaaacgagtacaagagcgagggcaccttcggcatccacgtggccgagaatcctagcatccaactgctggaaggcaaccccggacctcctgctgatccagatggtggacctagacctcaggccgaccggaagttccaagaaagagaggtgccctgccaccggccatctccaggtgcactttggctgcaagtggctgtgacaggcgtgctggtggttacactgctggtcgtgctgtacagaaggcggctgcattga

SEQ ID NO: 251 - RNA, 1395 nt, Artificial Sequence
  Codon optimised RNA encoding MAVS
augggcugugaacugguggaucuggccgaugaaguggccagcguguaccagagcuaccagccuagaaccagcgaccggccuccugauccucuggaaccuccaucucugcccgccgaaagaccuggaccuccuacaccagcugccgcucacagcaucccuuacaacagcugcagagagaaagaaccuagcuaccccaugccugugcaagagacacaggccccagaaagcccuggcgagaauucugaacaggcccugcagacacugagccccagagccauuccuagaaacccugauggcggcccucuggaaagcagcagugaucuggcugcucugagcccucugacaagcucuggacaccaagagcaggauaccgagcugggcagcacacauacagccggcgcuacaagcagccugacaccuucuagaggccccgugucucccagcgugucauuucagccucuggccaggucuaccccuagagccucuagacugccuggaccuacaggcagcguggugucuaccggcacaagcuucagcucuagcucuccuggacuggccucugcuggugccgcugagggaaaacaaggcgccgaaucugaucaggccgagccuaucaucuguagcagcggagcagaagccccugccaauagccugccuagcaaggugccaaccacacugaugcccgugaacacaguggcccugaaggugccagcuaauccugccuccguguccaccgugccuucuaagcugccaaccagcucuaagccaccuggcgccgugccaucuaacgcccugacaaauccugcuccaagcaagcugcccaucaacagcacaagagccggcauggugcccucuaaggugcccacaucuauggugcugaccaagguguccgccagcaccgugccaacagauggcagcagcagaaacgaggaaaccccugccgcuccuacuccugcuggcgcuacaggcggaucuucugccuggcuggauagcagcuccgagaauagaggccugggcagcgagcugucuaaaccuggcguucuggcaagccagguggacagcccuuucagcggcugcuuugaggaccuggcuaucagcgccucuacaagccucggcaugggaccuugucacggccccgaggaaaacgaguacaagagcgagggcaccuucggcauccacguggccgagaauccuagcauccaacugcuggaaggcaaccccggaccuccugcugauccagaugguggaccuagaccucaggccgaccggaaguuccaagaaagagaggugcccugccaccggccaucuccaggugcacuuuggcugcaaguggcugugacaggcgugcuggugguuacacugcuggucgugcuguacagaaggcggcugcauuga

SEQ ID NO: 252 - protein, 493 aa, Homo sapiens
MERSPDVSPGPSRSFKEELLCAVCYDPFRDAVTLRCGHNFCRGCVSRCWEVQVSPTCPVCKDRASPADLRTNHTLNNLVEKLLREEAEGARWTSYRFSRVCRLHRGQLSLFCLEDKELLCCSCQADPRHQGHRVQPVKDTAHDFRAKCRNMEHALREKAKAFWAMRRSYEAIAKHNQVEAAWLEGRIRQEFDKLREFLRVEEQAILDAMAEETRQKQLLADEKMKQLTEETEVLAHEIERLQMEMKEDDVSFLMKHKSRKRRLFCTMEPEPVQPGMLIDVCKYLGSLQYRVWKKMLASVESVPFSFDPNTAAGWLSVSDDLTSVTNHGYRVQVENPERFSSAPCLLGSRVFSQGSHAWEVALGGLQSWRVGVVRVRQDSGAEGHSHSCYHDTRSGFWYVCRTQGVEGDHCVTSDPATSPLVLAIPRRLRVELECEEGELSFYDAERHCHLYTFHARFGEVRPYFYLGGARGAGPPEPLRICPLHISVKEELDG

SEQ ID NO: 253 - DNA, 1479 bp, Homo sapiens
atggagcggagtcccgacgtgtcccccgggccttcccgctccttcaaggaggagttgctctgcgccgtctgctacgaccccttccgcgacgcagtcactctgcgctgcggccacaacttctgccgcgggtgcgtgagccgctgctgggaggtgcaggtgtcgcccacctgcccagtgtgcaaagaccgcgcgtcacccgccgacctgcgcaccaaccacaccctcaacaacctggtggagaagctgctgcgcgaggaggccgagggcgcgcgctggaccagctaccgcttctcgcgtgtctgccgcctgcaccgcggacagctcagcctcttctgcctcgaggacaaggagctgctgtgctgctcctgccaggccgacccccgacaccaggggcaccgcgtgcagccggtgaaggacactgcccacgactttcgggccaagtgcaggaacatggagcatgcactgcgggagaaggccaaggccttctgggccatgcggcgctcctatgaggccatcgccaagcacaatcaggtggaggctgcatggctggaaggccggatccggcaggagtttgataagcttcgcgagttcttgagagtggaggagcaggccattctggatgccatggccgaggagacaaggcagaagcaacttctggccgacgagaagatgaagcagctcacagaggagacggaggtgctggcacatgagatcgagcggctgcagatggagatgaaggaggacgacgtttcttttctcatgaaacacaagagccgaaaacgccgactcttctgcaccatggagccagagccagtccagcccggcatgcttatcgatgtctgcaagtacctgggctccctgcagtaccgcgtctggaagaagatgcttgcatctgtggaatctgtacccttcagctttgaccccaacaccgcagctggctggctctccgtgtctgacgacctcaccagcgtcaccaaccatggctaccgcgtgcaggtggagaacccggaacgcttctcctcggcgccctgcctgctgggctcccgtgtcttctcacagggctcgcacgcctgggaggtggcccttggggggctgcagagctggagggtgggcgtggtacgtgtgcgccaggactcgggcgctgagggccactcacacagctgctaccacgacacacgctcgggcttctggtatgtctgccgcacgcagggcgtggagggggaccactgcgtgacctcggacccagccacgtcgcccctggtcctggccatcccacgccgcctgcgtgtggagctggagtgtgaggagggcgagctgtctttctatgacgcggagcgccactgccacctgtacaccttccacgcccgctttggggaggttcgcccctacttctacctggggggtgcacggggcgccgggcctccagagcctttgcgcatctgccccttgcacatcagtgtcaaggaagaactggatggc

SEQ ID NO: 254 - RNA, 1479 nt, Homo sapiens
auggagcggagucccgacgugucccccgggccuucccgcuccuucaaggaggaguugcucugcgccgucugcuacgaccccuuccgcgacgcagucacucugcgcugcggccacaacuucugccgcgggugcgugagccgcugcugggaggugcaggugucgcccaccugcccagugugcaaagaccgcgcgucacccgccgaccugcgcaccaaccacacccucaacaaccugguggagaagcugcugcgcgaggaggccgagggcgcgcgcuggaccagcuaccgcuucucgcgugucugccgccugcaccgcggacagcucagccucuucugccucgaggacaaggagcugcugugcugcuccugccaggccgacccccgacaccaggggcaccgcgugcagccggugaaggacacugcccacgacuuucgggccaagugcaggaacauggagcaugcacugcgggagaaggccaaggccuucugggccaugcggcgcuccuaugaggccaucgccaagcacaaucagguggaggcugcauggcuggaaggccggauccggcaggaguuugauaagcuucgcgaguucuugagaguggaggagcaggccauucuggaugccauggccgaggagacaaggcagaagcaacuucuggccgacgagaagaugaagcagcucacagaggagacggaggugcuggcacaugagaucgagcggcugcagauggagaugaaggaggacgacguuucuuuucucaugaaacacaagagccgaaaacgccgacucuucugcaccauggagccagagccaguccagcccggcaugcuuaucgaugucugcaaguaccugggcucccugcaguaccgcgucuggaagaagaugcuugcaucuguggaaucuguacccuucagcuuugaccccaacaccgcagcuggcuggcucuccgugucugacgaccucaccagcgucaccaaccauggcuaccgcgugcagguggagaacccggaacgcuucuccucggcgcccugccugcugggcucccgugucuucucacagggcucgcacgccugggagguggcccuuggggggcugcagagcuggagggugggcgugguacgugugcgccaggacucgggcgcugagggccacucacacagcugcuaccacgacacacgcucgggcuucugguaugucugccgcacgcagggcguggagggggaccacugcgugaccucggacccagccacgucgccccugguccuggccaucccacgccgccugcguguggagcuggagugugaggagggcgagcugucuuucuaugacgcggagcgccacugccaccuguacaccuuccacgcccgcuuuggggagguucgccccuacuucuaccuggggggugcacggggcgccgggccuccagagccuuugcgcaucugccccuugcacaucagugucaaggaagaacuggauggc

SEQ ID NO: 255 - DNA, 1482 bp, Artificial Sequence
  Codon optimised DNA encoding TRIM35
atggaaagatcccctgacgtgtcccctggacctagcagaagcttcaaagaggaactgctctgcgccgtgtgctacgaccccttcagagatgccgtgacactgagatgcggccacaacttctgcagaggctgcgtgtccagatgctgggaagtgcaggtttcccctacatgccccgtgtgcaaggacagagcctctcctgccgatctgcggaccaatcacaccctgaacaacctggtggaaaagctgctgagagaagaggccgaaggcgccagatggaccagctacagattcagcagagtgtgccggctgcacagaggccagctgagcctgttctgtctcgaggacaaagaactgctgtgctgcagctgccaggccgatcctagacaccagggacatagagtgcagcccgtgaaggacacagcccacgacttcagagccaagtgccggaacatggaacacgccctgagagagaaggccaaagccttctgggccatgcggagaagctatgaggccattgccaagcacaatcaggtggaagccgcctggctggaaggccggatcagacaagagttcgacaagctgcgcgagttcctgagagtggaagaacaggccatcctggacgccatggccgaggaaacaagacagaaacagctgctggccgacgagaagatgaagcagctgaccgaagagacagaggtgctggcccacgaaatcgagcggctgcagatggaaatgaaggaagatgatgtgtcctttctgatgaagcacaagagccggaagcggcggctgttctgcacaatggaacctgagccagtgcagcctggcatgctgatcgatgtgtgcaagtacctgggcagcctgcagtacagagtgtggaagaaaatgctggcctccgtggaaagcgtgcccttcagcttcgaccctaatactgccgctggctggctgagcgtgtccgatgatctgaccagcgtgaccaaccacggctacagagtgcaggtcgagaaccccgagagattcagctctgccccttgtctgctgggctccagagtgttttctcagggctctcacgcctgggaagttgcccttggaggactccagtcttggagagtgggcgttgtcagagtgcggcaggattctggcgccgaaggacactctcacagctgctaccacgatacccgcagcggcttttggtacgtgtgtagaacacagggcgtcgagggcgaccactgtgtgacatctgaccctgccacatctcctctggtgctggctatccctcggagactgagagtcgagctggaatgcgaggaaggcgagctgagcttctacgacgccgagagacactgccacctgtacaccttccacgccagatttggcgaagtgcggccctacttttatctcggcggagctagaggtgccggacctcctgaacctctgagaatctgccctctgcacatcagcgtgaaagaggaattggacggctga

SEQ ID NO: 256 - RNA, 1482 nt, Artificial Sequence
  Codon optimised RNA encoding TRIM35
auggaaagauccccugacguguccccuggaccuagcagaagcuucaaagaggaacugcucugcgccgugugcuacgaccccuucagagaugccgugacacugagaugcggccacaacuucugcagaggcugcguguccagaugcugggaagugcagguuuccccuacaugccccgugugcaaggacagagccucuccugccgaucugcggaccaaucacacccugaacaaccugguggaaaagcugcugagagaagaggccgaaggcgccagauggaccagcuacagauucagcagagugugccggcugcacagaggccagcugagccuguucugucucgaggacaaagaacugcugugcugcagcugccaggccgauccuagacaccagggacauagagugcagcccgugaaggacacagcccacgacuucagagccaagugccggaacauggaacacgcccugagagagaaggccaaagccuucugggccaugcggagaagcuaugaggccauugccaagcacaaucagguggaagccgccuggcuggaaggccggaucagacaagaguucgacaagcugcgcgaguuccugagaguggaagaacaggccauccuggacgccauggccgaggaaacaagacagaaacagcugcuggccgacgagaagaugaagcagcugaccgaagagacagaggugcuggcccacgaaaucgagcggcugcagauggaaaugaaggaagaugauguguccuuucugaugaagcacaagagccggaagcggcggcuguucugcacaauggaaccugagccagugcagccuggcaugcugaucgaugugugcaaguaccugggcagccugcaguacagaguguggaagaaaaugcuggccuccguggaaagcgugcccuucagcuucgacccuaauacugccgcuggcuggcugagcguguccgaugaucugaccagcgugaccaaccacggcuacagagugcaggucgagaaccccgagagauucagcucugccccuugucugcugggcuccagaguguuuucucagggcucucacgccugggaaguugcccuuggaggacuccagucuuggagagugggcguugucagagugcggcaggauucuggcgccgaaggacacucucacagcugcuaccacgauacccgcagcggcuuuugguacguguguagaacacagggcgucgagggcgaccacugugugacaucugacccugccacaucuccucuggugcuggcuaucccucggagacugagagucgagcuggaaugcgaggaaggcgagcugagcuucuacgacgccgagagacacugccaccuguacaccuuccacgccagauuuggcgaagugcggcccuacuuuuaucucggcggagcuagaggugccggaccuccugaaccucugagaaucugcccucugcacaucagcgugaaagaggaauuggacggcuga

SEQ ID NO: 257 - protein, 129 aa, Homo sapiens
MNLEGGGRGGEFGMSAVSCGNGKLRQWLIDQIDSGKYPGLVWENEEKSIFRIPWKHAGKQDYNREEDAALFKAWALFKGKFREGIDKPDPPTWKTRLRCALNKSNDFEELVERSQLDISDPYKVYRIVP

SEQ ID NO: 258 - DNA, 387 bp, Homo sapiens
atgaacctggagggcggcggccgaggcggagagttcggcatgagcgcggtgagctgcggcaacgggaagctccgccagtggctgatcgaccagatcgacagcggcaagtaccccgggctggtgtgggagaacgaggagaagagcatcttccgcatcccctggaagcacgcgggcaagcaggactacaaccgcgaggaggacgccgcgctcttcaaggcttgggcactgtttaaaggaaagttccgagaaggcatcgacaagccggaccctcccacctggaagacgcgcctgcggtgcgctttgaacaagagcaatgactttgaggaactggttgagcggagccagctggacatctcagacccgtacaaagtgtacaggattgttcct

SEQ ID NO: 259 - RNA, 387 nt, Homo sapiens
augaaccuggagggcggcggccgaggcggagaguucggcaugagcgcggugagcugcggcaacgggaagcuccgccaguggcugaucgaccagaucgacagcggcaaguaccccgggcuggugugggagaacgaggagaagagcaucuuccgcauccccuggaagcacgcgggcaagcaggacuacaaccgcgaggaggacgccgcgcucuucaaggcuugggcacuguuuaaaggaaaguuccgagaaggcaucgacaagccggacccucccaccuggaagacgcgccugcggugcgcuuugaacaagagcaaugacuuugaggaacugguugagcggagccagcuggacaucucagacccguacaaaguguacaggauuguuccu

SEQ ID NO: 260 - DNA, 390 bp, Artificial Sequence
  Codon optimised DNA encoding IRF4
atgaatctggaaggcggcggaagaggcggcgagtttggaatgtctgccgtgtcctgtggcaacggcaagctgagacagtggctgatcgaccagatcgacagcggcaagtatcctggcctcgtgtgggagaacgaggaaaagtctatcttcagaatcccctggaagcacgccggcaagcaggactacaacagagaagaggacgccgctctgttcaaggcctgggctctgtttaagggcaagttcagagagggcatcgacaagcccgatcctccaacctggaaaaccagactgagatgcgccctgaacaagagcaacgacttcgaggaactggtggaaagaagccagctggacatcagcgacccctacaaggtgtaccggatcgtgccctga

SEQ ID NO: 261 - RNA, 390 nt, Artificial Sequence
  Codon optimised RNA encoding IRF4
augaaucuggaaggcggcggaagaggcggcgaguuuggaaugucugccguguccuguggcaacggcaagcugagacaguggcugaucgaccagaucgacagcggcaaguauccuggccucgugugggagaacgaggaaaagucuaucuucagaauccccuggaagcacgccggcaagcaggacuacaacagagaagaggacgccgcucuguucaaggccugggcucuguuuaagggcaaguucagagagggcaucgacaagcccgauccuccaaccuggaaaaccagacugagaugcgcccugaacaagagcaacgacuucgaggaacugguggaaagaagccagcuggacaucagcgaccccuacaagguguaccggaucgugcccuga

SEQ ID NO: 262 - DNA, 522 bp, Artificial Sequence
  Codon optimised DNA encoding ARL16
atgtgtctgctgctgggagctacaggcgtgggcaagacactgctggtcaagcggctgcaagaggtgtccagcagagatggcaaaggcgatctgggagagcctcctccaaccagacctaccgtgggcaccaacctgacagatatcgtggcccagcggaagatcaccatcagagaactcggcggctgcatgggccctatctggtctagctactacggcaactgccgcagcctgctgttcgtgatggatgccagcgatcccacacagctgagcgcctcttgtgtgcaactgctgggactgctgtctgccgaacaactggccgaagcctctgtgctgatcctgttcaacaagatcgacctgccttgctacatgagcaccgaggaaatgaagtccctgatcagactgcccgacatcattgcctgcgccaagcagaatatcaccacagccgagatcagcgccagagaaggcacaggacttgctggcgttctggcatggctgcaggccacacacagagccaacgattga

SEQ ID NO: 263 - RNA, 522 nt, Artificial Sequence
  Codon optimised RNA encoding ARL16
augugucugcugcugggagcuacaggcgugggcaagacacugcuggucaagcggcugcaagagguguccagcagagauggcaaaggcgaucugggagagccuccuccaaccagaccuaccgugggcaccaaccugacagauaucguggcccagcggaagaucaccaucagagaacucggcggcugcaugggcccuaucuggucuagcuacuacggcaacugccgcagccugcuguucgugauggaugccagcgaucccacacagcugagcgccucuugugugcaacugcugggacugcugucugccgaacaacuggccgaagccucugugcugauccuguucaacaagaucgaccugccuugcuacaugagcaccgaggaaaugaagucccugaucagacugcccgacaucauugccugcgccaagcagaauaucaccacagccgagaucagcgccagagaaggcacaggacuugcuggcguucuggcauggcugcaggccacacacagagccaacgauuga

SEQ ID NO: 264 - RNA, 796 nt, Artificial Sequence
  RNA construct
cggagacggcgcagaagaagaggaucuggcgaaggcagaggcagccugcuuacauguggcgacguggaagagaaccccggaccuaugggcgauagcagccccgauaccuuuuccgauggccugagcagcagcacccugccugaugaucacagcagcuacaccgugccuggcuacaugcaggaccuggaaguggaacaggcccugacaccagcucugagcccuugugcuguguccagcacacugcccgauuggcacaucccuguggaaguggugccugacagcaccagcgaccuguacaacuuccaaguguccccuaugccuagcaccuccgaggccaccaccgaugaggaugaagagggaaagcugcccgaggacaucaugaagcugcuggaacagagcgaguggcagcccaccaauguggauggcaagggcuaccugcugaacgagccuggcguucagccuacaagcguguacggcgacuucagcugcaaagaggaacccgagaucgauagcccuggcggcgauaucggacugagccugcagagaguguucaccgaccugaagaacauggacgccaccuggcuggacagccugcugacaccuguuagacugcccucuauccaggcuauccccugcgcuccuugagcggccgcgaauuggcaagcugcuuacauagaacucgcggcgauuggcaugccgccuuaaaauuuuuauuuuauuuuucuuuucuuuuccgaaucggauuuuguuuuuaauauuucaaaaaaaaaaaaaaaaaaaaaaaaaaaaaaaaaaa

SEQ ID NO: 265 - DNA, 796 bp, Artificial Sequence
  Nucleic acid sequence encoding RNA construct
cggagacggcgcagaagaagaggatctggcgaaggcagaggcagcctgcttacatgtggcgacgtggaagagaaccccggacctatgggcgatagcagccccgataccttttccgatggcctgagcagcagcaccctgcctgatgatcacagcagctacaccgtgcctggctacatgcaggacctggaagtggaacaggccctgacaccagctctgagcccttgtgctgtgtccagcacactgcccgattggcacatccctgtggaagtggtgcctgacagcaccagcgacctgtacaacttccaagtgtcccctatgcctagcacctccgaggccaccaccgatgaggatgaagagggaaagctgcccgaggacatcatgaagctgctggaacagagcgagtggcagcccaccaatgtggatggcaagggctacctgctgaacgagcctggcgttcagcctacaagcgtgtacggcgacttcagctgcaaagaggaacccgagatcgatagccctggcggcgatatcggactgagcctgcagagagtgttcaccgacctgaagaacatggacgccacctggctggacagcctgctgacacctgttagactgccctctatccaggctatcccctgcgctccttgagcggccgcgaattggcaagctgcttacatagaactcgcggcgattggcatgccgccttaaaatttttattttatttttcttttcttttccgaatcggattttgtttttaatatttcaaaaaaaaaaaaaaaaaaaaaaaaaaaaaaaaaaa

SEQ ID NO: 266 - DNA, 2661 bp, Artificial Sequence
  Recombinant vector
cggagacggcgcagaagaagaggatctggcgaaggcagaggcagcctgcttacatgtggcgacgtggaagagaaccccggacctatgggcgatagcagccccgataccttttccgatggcctgagcagcagcaccctgcctgatgatcacagcagctacaccgtgcctggctacatgcaggacctggaagtggaacaggccctgacaccagctctgagcccttgtgctgtgtccagcacactgcccgattggcacatccctgtggaagtggtgcctgacagcaccagcgacctgtacaacttccaagtgtcccctatgcctagcacctccgaggccaccaccgatgaggatgaagagggaaagctgcccgaggacatcatgaagctgctggaacagagcgagtggcagcccaccaatgtggatggcaagggctacctgctgaacgagcctggcgttcagcctacaagcgtgtacggcgacttcagctgcaaagaggaacccgagatcgatagccctggcggcgatatcggactgagcctgcagagagtgttcaccgacctgaagaacatggacgccacctggctggacagcctgctgacacctgttagactgccctctatccaggctatcccctgcgctccttgagcggccgcgaattggcaagctgcttacatagaactcgcggcgattggcatgccgccttaaaatttttattttatttttcttttcttttccgaatcggattttgtttttaatatttcaaaaaaaaaaaaaaaaaaaaaaaaaaaaaaaaaaacgcgtcgaggggaattaattcttgaagacgaaagggccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccattctagaatggcgcgcccttaaggggagaataggagccgcaacacacaagcaacgcgaggtcgtttaaac

(19) United States
(12) Patent Application Publication
(10) Pub. No.: US 2024/0124530 A1
(43) Pub. Date: Apr. 18, 2024

(54) RNA CONSTRUCT

(71) Applicants: Imperial College Innovations Limited, London (GB); VaxEquity Ltd, Little Chesterford (GB)

(72) Inventors: Robin Shattock, London (GB); Paul McKay, London (GB); Michael Watson, Little Chesterford (GB); Elaine Harper, Little Chesterford (GB)

(73) Assignees: Imperial College Innovations Limited, London (GB); VaxEquity Ltd, Little Chesterford, ES (GB)

(21) Appl. No.: 18/257,698

(22) PCT Filed: Dec. 17, 2021
(86) PCT No.: PCT/GB2021/053361

§ 371 Date: Jun. 15, 2023

(30) Foreign Application Priority Data
Dec. 17, 2020 (GB) 2020063.0

(51) Int. Cl. C07K 14/005; A61P 37/02

(52) U.S. Cl. CPC C07K 14/005; A61P 37/02

(57) ABSTRACT

The present invention relates to RNA constructs, and particularly, although not exclusively, to mRNA constructs and saRNA replicons and to nucleic acids and expression vectors encoding such RNA constructs. The invention extends to the use of such RNA constructs in therapy, for example in treating diseases and/or in vaccine delivery. The invention extends to pharmaceutical compositions comprising such RNA constructs, and methods and uses thereof.

The present invention relates to RNA constructs, and particularly, although not exclusively, to mRNA constructs and saRNA replicons and to nucleic acids and expression vectors encoding such RNA constructs. The invention extends to the use of such RNA constructs in therapy, for example in treating diseases and/or in vaccine delivery. The invention extends to pharmaceutical compositions comprising such RNA constructs, and methods and uses thereof.

Messenger RNA (mRNA) is a promising tool for biotherapeutics. However, while mRNA therapeutics have been shown to be highly effective in small animals, the outcomes do not scale linearly when these formulations are translated in dose-escalation studies in humans. Furthermore, adverse events associated with the induction of interferon responses have been rate-limiting with respect to the increased doses of RNA likely to be effective in humans. The reason for this inconsistency is unclear, but the inventors hypothesize that inherent differences in human innate sensing pose a barrier to the translation of RNA therapeutics from the lab to the clinic. Furthermore, innate sensing of RNA has been associated with the inhibition of protein expression. To date, the main approach to overcoming the innate recognition of exogenous RNA has been to use modified ribonucleotides that are less detectable by innate sensing mechanisms. However, modified mRNA is not completely undetectable, and still results in some induction of interferon, protein silencing and reduced tolerability for human use (see FIG. 2).

Another approach has been the use of self-amplifying or saRNA vectors, which are typically based on an alphavirus backbone that have the capacity to self-amplify their own RNA by encoding polymerase activity within their non-structural proteins. Prior art methods have involved replacing the structural proteins of these vectors by a gene of interest (GOI), for example encoding an antigen of interest be it a vaccine construct or encoding a therapeutic protein. Other versions of saRNA have been based on picornaviruses, flaviviruses, and coronaviruses. When saRNA is taken up into the cytoplasm of target cells, this leads to amplification of the RNA by the encoded polymerase machinery and very high expression levels of the GOI. As a consequence, saRNA has been shown to induce immune responses with lower doses of saRNA than mRNA (10- to 100-fold lower) and results in prolonged protein expression for up to 60 days in mice.

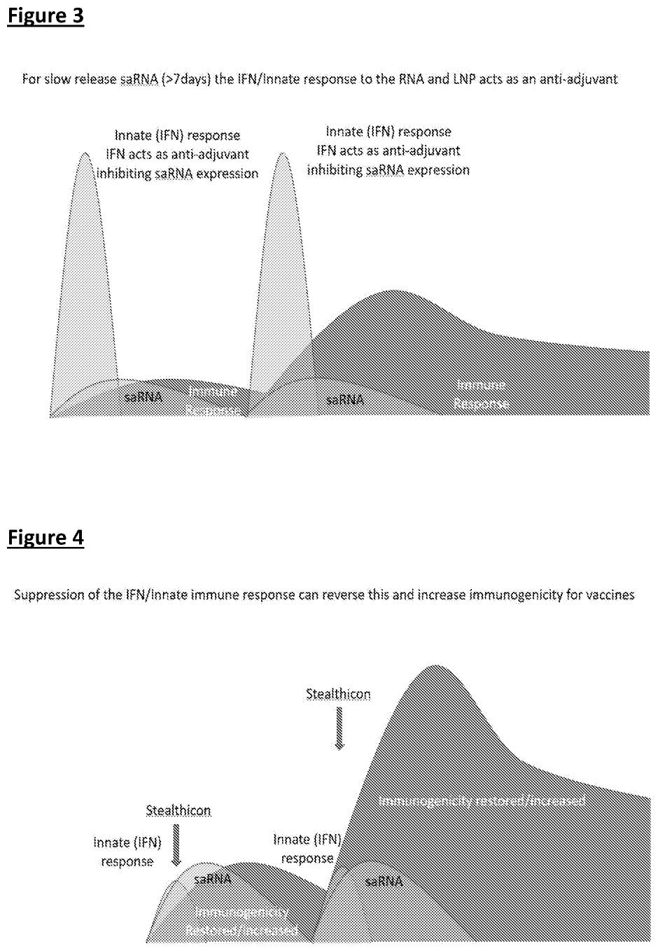

However, as shown in FIG. 3, a drawback with saRNA is that it is also sensed by innate sensing pattern recognition receptors, triggering antiviral (interferon) responses that limit protein expression and self-amplification of these prior art saRNAs. Innate sensing of saRNA differs to that of mRNA due to its large size (typically >5000 bases) and profound secondary structure, including double stranded regions (dsRNA). Long and double stranded RNA triggers innate responses through, among other sensors, the MDA5 (Melanoma Differentiation-Associated protein 5) pathway. This is facilitated by the binding of PACT (PKR activating protein) to long and dsRNA RNA promoting the oligomerization of MDA5 and subsequent triggering of a down-stream signalling cascade that inhibits replication and expression of saRNA.

Accordingly, there is a need in the art to produce new means by which RNA therapeutics, be they mRNA- or saRNA-based can be delivered and expressed in patients, such that they are able to overcome the innate immune system sensing.

The inventors have developed a novel RNA construct (saRNA and mRNA) that advantageously overcomes the innate immune system which senses RNA, by expressing non-viral (e.g. mammalian) immune modulating proteins that block or reduce the activity of immune system machinery, resulting in improved translation (in the case of mRNA) and increased self-amplification and subsequent translation (in the case of saRNA), and therefore greater protein expression levels of the gene of interest, such as an antigen, in a host cell.

Accordingly, in a first aspect of the invention, there is provided an RNA construct encoding: (i) at least one therapeutic biomolecule; and (ii) at least one non-viral innate modulatory protein (IMP).

RNA constructs, such as mRNA and saRNA replicons have been postulated to be potential tools for the delivery and expression of genes of interest for vaccines and therapeutics. However, single stranded mRNA (ssRNA) and double stranded RNA (dsRNA) is detected intracellularly by innate sensing mechanisms that trigger responses, which inhibit protein translation. As a consequence, expression of genes of interest encoded by the RNA construct is significantly impaired and thus the immunogenic or therapeutic potential of RNA construct, including saRNA and mRNA, is limited. Advantageously, the RNA constructs of the invention overcome this problem because they encode one or more non-viral innate modulatory protein (IMP), which reduces or ablates the downstream innate inhibition of transgene expression within the host cell.

The induction of interferon is one downstream consequence of innate recognition, but it will be appreciated that other molecules and pathways can and are induced, as discussed below, and any of these will be inhibited by the one or more non-viral immune modulating protein that is harboured in the RNA construct. Preferably, therefore, the at least one innate modulatory protein (IMP) is capable of modulating the innate immune response to RNA in a subject treated with the RNA construct of the invention. The IMP can therefore be described as a modulator of innate immunity. It may also be described as an interferon inhibiting molecule in some embodiments.

One previously published approach to ablating the interferon response with saRNA used interferon inhibiting proteins from the vaccinia virus, E3, K3 and B18. However, in that study, the interferon inhibiting proteins were delivered and formulated as separate mRNA molecules that were combined with the saRNA. This requires the manufacture of both saRNA and mRNA, and necessitated the use of at least 3-6 times as much vaccinia mRNA as the saRNA replicon construct according to the invention provide any observable enhancement in protein expression.

Advantageously, the presence, in the RNA construct of the first aspect, of one or more non-viral innate modulatory protein, enables dual protein expression with the peptide or protein of interest, i.e. the biotherapeutic molecule. As opposed to delivering two different strands of RNA as described in the prior art, one encoding the peptide/protein of interest and one encoding the innate modulatory protein, when using the RNA construct of the invention, only one single strand is delivered to the target cell, thereby ensuring colocalization of the RNA molecule and the non-viral immune modulating protein. The non-viral immune modulating protein inhibits the innate sensing of RNA in the host cell, thereby enabling higher protein expression and translation, and the non-viral immune modulating protein expression itself is co-expressed and translated from the same RNA molecule as the therapeutic biomolecule.

As described in the examples, the RNA constructs of the invention (also known as “Stealthicons”) encoding exemplar luciferase (as a GOI) have surprisingly been shown to increase luciferase protein expression levels of at least two orders of magnitude and greater in a human cell line with intact innate sensing systems in vitro compared to the control lacking an IMP in the construct, and also to increase both the magnitude and duration of protein expression of luciferase compared to a conventional VEEV RNA replicon in vivo in BL/6 mice. In addition, VEGF-A (see FIG. 10) represents an alternative exemplar to luciferase as the GOI.

The skilled person would readily appreciate that the luciferase reporter is truly representative of the therapeutic biomolecule described herein (i.e. the GOI), because it proves that the RNA construct is able to express in vivo the gene harboured on the RNA molecule of the invention. As such, the luciferase provides robust evidence of the proof of concept that the RNA construct of the invention can be used to express any therapeutically active biomolecule, such as an antigen for triggering an immune response.

The RNA construct of the first aspect may be single-stranded RNA or double-stranded RNA.

The RNA construct may comprise mRNA or saRNA.

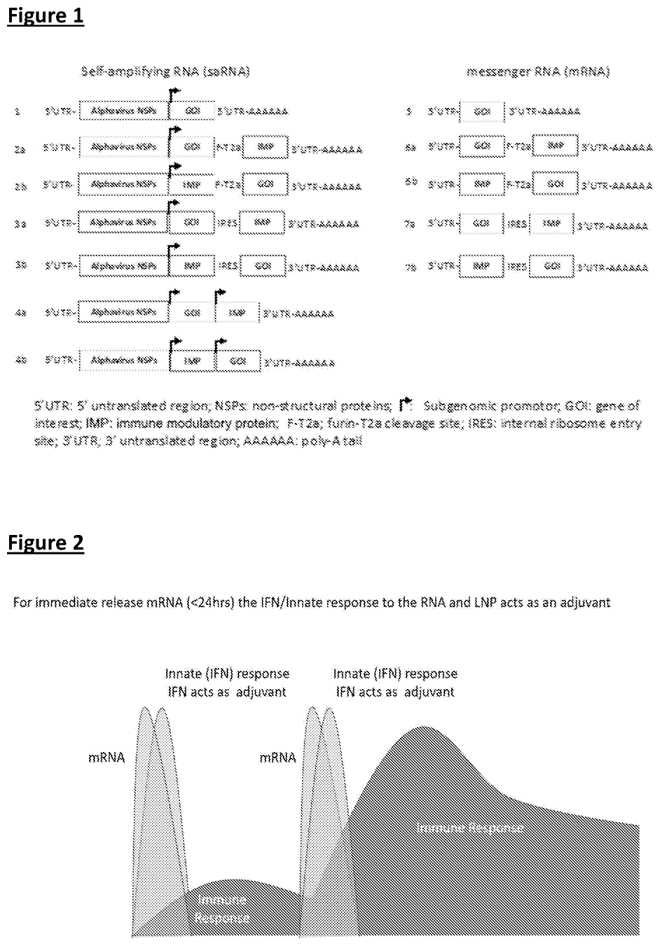

In one embodiment, the RNA construct comprises mRNA. FIG. 1 (right hand side) illustrates various embodiments of the RNA construct as a mRNA molecule.

In a preferred embodiment, however, the RNA construct comprises self-amplifying RNA (saRNA). FIG. 1 (left hand side) illustrates various embodiments of the RNA construct as a saRNA molecule. The skilled person would understand that such an RNA construct can also be referred to as a self-replicating RNA virus vector, or an RNA replicon.

Preferably, the saRNA construct comprises or is derived from a positive stranded RNA virus selected from the group of genus consisting of: alphavirus; picornavirus; flavivirus; rubivirus; pestivirus; hepacivirus; calicivirus and coronavirus.

Preferably, the RNA construct comprises or is derived from an alphavirus. Suitable wild-type alphavirus sequences are well-known. Representative examples of suitable alphaviruses include Aura, Bebaru virus, Cabassou, Chikungunya virus, Eastern equine encephalomyelitis virus, Fort Morgan, Getah virus, Kyzylagach, Mayaro, Mayaro virus, Middleburg, Mucambo virus, Ndumu, Pixuna virus, Ross River virus, Semliki Forest, Sindbis virus, Tonate, Triniti, Una, Venezuelan equine encephalomyelitis, Western equine encephalomyelitis, Whataroa, and Y-62-33. Preferably, therefore, the RNA construct comprises or is derived from any of these alphaviruses.

Preferably, the RNA construct comprises or is derived from a virus selected from the group of species consisting of: Venezuelan Equine Encephalitis Virus (VEEV); enterovirus 71; Encephalomyocarditis virus; Kunjin virus; and Middle East respiratory syndrome virus. In one preferred embodiment, the RNA construct comprises or is derived from Kunjin virus. Preferably, the RNA construct comprises or is derived from VEEV.

Preferably, the RNA construct comprises a nucleotide sequence, which encodes the at least one innate modulatory protein (IMP), which is capable of reducing, ablating or blocking the innate immune response to RNA. The IMP is, therefore, a modulator of innate immunity. It may also be an interferon inhibitor of interferon signalling.

The IMP is preferably a mammalian IMP. More preferably, the IMP is a primate IMP. Most preferably, the IMP is a human IMP.

The reduction, ablation or blocking of the innate immune response to RNA in a host cell transformed with that RNA molecule (i.e. non-endogenously produced RNA) may be achieved by the IMP regulating interferon production, inhibiting innate signalling pathways, and/or inhibiting RNA recognition. It will be appreciated that regulation of interferon production could be described as inhibiting innate signalling. Therefore, innate sensing and innate signalling systems include: (a) RNA recognition systems, (b) pathways leading to interferon production and resulting in stimulation of interferon-stimulated genes, and (c) interferon signalling systems.

The IMP may, therefore, fall into any of the following four broad categories:—
(i) Category 1: Inhibitors of interferon regulatory factor activity;
(ii) Category 2: Inhibitors of pathways leading to interferon production and resulting in stimulation of interferon-stimulated genes;
(iii) Category 3: Inhibitors of interferon signalling; and/or
(iv) Category 4: Inhibitors of RNA recognition systems.

It will be appreciated that some IMPs may have multiple actions. For instance, a Category 4 IMP may also be classified as a Category 2 IMP (e.g. IRF3, IRF7) and a Category 3 IMP (e.g. IRF9).

Category 1: Inhibitors of Interferon Regulatory Factor Activity

In one embodiment, the IMP may be configured to inhibit interferon regulatory factor activity.

The reduction, ablation or blocking of the innate immune response to RNA is preferably achieved by the IMP by reducing or preventing the activation of interferon regulatory factors (e.g. IRF3 and IRF7), NF-κB transcription factors and other signalling proteins which directly trigger a range of antiviral genes (e.g. IFIT1-3, Mx1, Mx2 known to suppress RNA expression), proinflammatory genes whose products orchestrate the innate immune response, and direct activation of canonically IFN-stimulated genes (ISGs) upstream of any interferon dependent cascade. These pathways may be enhanced by the induction of type I & III interferons that provide a positive feedback loop further amplifying many antiviral responses.

Preferably, therefore, the innate modulatory protein encoded by the RNA construct comprises a mutated or non-functional interferon regulatory factor (IRF), or a dominant negative acting form thereof. The IRF, or the dominant negative form thereof, is preferably mutated such that it competes with, or prevents binding of RNA to, the native IRF in the host cell.

The mutated or non-functional interferon regulatory factor, or dominant negative acting form thereof, may be any one of IRF1, IRF2, IRF3, IRF4, IRF5, IRF6, IRF7, IRF8, or IRF9.

In one embodiment, any of the IRFs described herein may comprise the whole protein, except for the deletion or mutation of either its DNA binding domain (DBD), and/or of its Nuclear Location Signal (NLS), such that the DBD and/or NLS is either non-functional or absent. Accordingly, preferably the innate modulatory protein encoded by the RNA construct comprises an interferon regulatory factor (IRF), which has had its DNA binding domain (DBD) and/or Nuclear Location Signal (NLS) rendered non-functional or deleted, so that it becomes a dominant negative form in the cytoplasm.

In yet another embodiment, preferably the innate modulatory protein encoded by the RNA construct that comprises the DBD and/or NLS of the IRF (either individually or fused together) competitively blocks binding of the corresponding native IRF to the promotor of one or more interferon stimulated gene (ISG).

Therefore, the mutated or non-functional interferon regulatory factor, or dominant negative acting form thereof, may comprise or consist of the DNA binding domain (DBD) and/or the Nuclear Location Signal (NLS) of an interferon regulatory factor (IRF).

The protein, DNA and RNA sequences for each of these IMPs is provided below. It will be appreciated that, for successful expression, the RNA construct preferably comprises a start codon such that the RNA is translated into the corresponding protein. Some of the IMPs provided below may not naturally have a start codon, and so for these, the RNA construct will need one adding to it at its 5′ end to ensure translation. Similarly, to ensure successful translation of the RNA into protein, a stop codon is required and, again, for some of the IMPs provided below, the RNA construct will require a stop codon at its 3′ end.

In an embodiment, the IRF may have had its DBD and/or NLS section deleted, rendering it a dominant negative form of the IRF which is unable to enter the nucleus. The at least one IMP may be a dominant negative form of IRF which may be selected from a group consisting of: IRF1 dominant negative; IRF3 dominant negative; IRF7 dominant negative; and IRF9 dominant negative.

In one embodiment, the at least one IMP may be an IRF1 dominant negative acting polypeptide (IRF1 (141-325)), i.e. IRF1 deleted of DBD and NLS (Accession Number—NCBI Reference Sequence: NM_002198.3; UniProtKB—P10914 (IRF1_HUMAN)), or an orthologue thereof. One embodiment of the polypeptide sequence of the IRF1 dominant negative form is represented herein as SEQ ID No: 1, as follows:

[SEQ ID No: 1]
GDSSPDTFSDGLSSSTLPDDHSSYTVPGYMQDLEVEQALT
PALSPCAVSSTLPDWHIPVEVVPDSTSDLYNFQVSPMPST
SEATTDEDEEGKLPEDIMKLLEQSEWQPTNVDGKGYLLNE
PGVQPTSVYGDFSCKEEPEIDSPGGDIGLSLQRVFTDLKN
MDATWLDSLLTPVRLPSIQAIPCAP

Therefore, preferably the RNA construct of the first aspect comprises a nucleotide sequence which encodes an amino acid sequence substantially as set out in SEQ ID No: 1, or a variant or fragment thereof. As shown in SEQ ID No: 1, the two highlighted (in bold) lysine (K) residues at positions 299 and 275 can be mutated to an arginine (R), as discussed below, to form a mutant IRF1 dominant negative acting polypeptide.

In one embodiment, the IRF1 dominant negative acting polypeptide is encoded by the DNA nucleotide sequence of SEQ ID No: 2, as follows:

[SEQ ID No: 2]
GGGGATTCCAGCCCTGATACCTTCTCTGATGGACTCAGCA
GCTCCACTCTGCCTGATGACCACAGCAGCTACACAGTTCC
AGGCTACATGCAGGACTTGGAGGTGGAGCAGGCCCTGACT
CCAGCACTGTCGCCATGTGCTGTCAGCAGCACTCTCCCCG
ACTGGCACATCCCAGTGGAAGTIGTGCCGGACAGCACCAG
TGATCTGTACAACTTCCAGGTGTCACCCATGCCCTCCACC
TCTGAAGCTACAACAGATGAGGATGAGGAAGGGAAATTAC
CTGAGGACATCATGAAGCTCTTGGAGCAGTCGGAGTGGCA
GCCAACAAACGTGGATGGGAAGGGGTACCTACTCAATGAA
CCTGGAGTCCAGCCCACCTCTGTCTATGGAGACTTTAGCT
GTAAGGAGGAGCCAGAAATTGACAGCCCAGGGGGGGATAT
TGGGCTGAGTCTACAGCGTGTCTTCACAGATCTGAAGAAC
ATGGATGCCACCTGGCTGGACAGCCTGCTGACCCCAGTCC
GGTTGCCCTCCATCCAGGCCATTCCCTGTGCACCGTAG

Accordingly, preferably the IRF1 dominant negative acting polypeptide is encoded by the DNA nucleotide sequence substantially as set out in SEQ ID NO: 2, or a variant or fragment thereof.

Thus, the RNA construct may comprise an RNA nucleotide sequence of SEQ ID No: 3, as follows:

[SEQ ID No: 3]
GGGGAUUCCAGCCCUGAUACCUUCUCUGAUGGACUCAGCA
GCUCCACUCUGCCUGAUGACCACAGCAGCUACACAGUUCC
AGGCUACAUGCAGGACUUGGAGGUGGAGCAGGCCCUGACU
CCAGCACUGUCGCCAUGUGCUGUCAGCAGCACUCUCCCCG
ACUGGCACAUCCCAGUGGAAGUUGUGCCGGACAGCACCAG
UGAUCUGUACAACUUCCAGGUGUCACCCAUGCCCUCCACC
UCUGAAGCUACAACAGAUGAGGAUGAGGAAGGGAAAUUAC
CUGAGGACAUCAUGAAGCUCUUGGAGCAGUCGGAGUGGCA
GCCAACAAACGUGGAUGGGAAGGGGUACCUACUCAAUGAA
CCUGGAGUCCAGCCCACCUCUGUCUAUGGAGACUUUAGCU
GUAAGGAGGAGCCAGAAAUUGACAGCCCAGGGGGGGAUAU
UGGGCUGAGUCUACAGCGUGUCUUCACAGAUCUGAAGAAC
AUGGAUGCCACCUGGCUGGACAGCCUGCUGACCCCAGUCC
GGUUGCCCUCCAUCCAGGCCAUUCCCUGUGCACCGUAG

Furthermore, preferably the RNA construct comprises an RNA nucleotide sequence substantially as set out in SEQ ID No: 3, or a variant or fragment thereof.

The inventors then subjected the modified protein sequence of SEQ ID No: 1 to codon optimisation for human expression, and one embodiment of the codon optimised nucleic acid (DNA) sequence that includes a start (ATG) and a stop (TGA) codon is provided herein as SEQ ID No: 4, as follows:

[SEQ ID No: 4]
ATGGGCGATAGCAGCCCCGATACCTTTTCCGATGGCCTGA
GCAGCAGCACCCTGCCTGATGATCACAGCAGCTACACCGT
GCCTGGCTACATGCAGGACCTGGAAGTGGAACAGGCCCTG
ACACCAGCTCTGAGCCCTTGTGCTGTGTCCAGCACACTGC
CCGATTGGCACATCCCTGTGGAAGTGGTGCCTGACAGCAC
CAGCGACCTGTACAACTTCCAAGTGTCCCCTATGCCTAGC
ACCTCCGAGGCCACCACCGATGAGGATGAAGAGGGAAAGC
TGCCCGAGGACATCATGAAGCTGCTGGAACAGAGCGAGTG
GCAGCCCACCAATGTGGATGGCAAGGGCTACCTGCTGAAC
GAGCCTGGCGTTCAGCCTACAAGCGTGTACGGCGACTTCA
GCTGCAAAGAGGAACCCGAGATCGATAGCCCTGGCGGCGA
TATCGGACTGAGCCTGCAGAGAGTGTTCACCGACCTGAAG
AACATGGACGCCACCTGGCTGGACAGCCTGCTGACACCTG
TTAGACTGCCCTCTATCCAGGCTATCCCCTGCGCTCCTTG
A

Hence, preferably the RNA construct is encoded by a DNA sequence substantially as set out in SEQ ID No: 4, or a fragment or variant thereof.

In an embodiment, the RNA sequence corresponding to the codon optimised DNA sequence of SEQ ID No: 4 that includes a start (AUG) and a stop (UGA) codon is provided herein as SEQ ID No: 5, as follows:

[SEQ ID No: 5]
AUGGGCGAUAGCAGCCCCGAUACCUUUUCCGAUGGCCUGA
GCAGCAGCACCCUGCCUGAUGAUCACAGCAGCUACACCGU
GCCUGGCUACAUGCAGGACCUGGAAGUGGAACAGGCCCUG
ACACCAGCUCUGAGCCCUUGUGCUGUGUCCAGCACACUGC
CCGAUUGGCACAUCCCUGUGGAAGUGGUGCCUGACAGCAC
CAGCGACCUGUACAACUUCCAAGUGUCCCCUAUGCCUAGC
ACCUCCGAGGCCACCACCGAUGAGGAUGAAGAGGGAAAGC
UGCCCGAGGACAUCAUGAAGCUGCUGGAACAGAGCGAGUG
GCAGCCCACCAAUGUGGAUGGCAAGGGCUACCUGCUGAAC
GAGCCUGGCGUUCAGCCUACAAGCGUGUACGGCGACUUCA
GCUGCAAAGAGGAACCCGAGAUCGAUAGCCCUGGCGGCGA
UAUCGGACUGAGCCUGCAGAGAGUGUUCACCGACCUGAAG
AACAUGGACGCCACCUGGCUGGACAGCCUGCUGACACCUG
UUAGACUGCCCUCUAUCCAGGCUAUCCCCUGCGCUCCUUG
A

Hence, preferably the RNA construct comprises a sequence substantially as set out in SEQ ID No: 5, or a fragment or variant thereof.

In another embodiment, the IRF1 dominant negative acting polypeptide of SEQ ID No:1 (Accession Number—NCBI Reference Sequence: NM_002198.3; UniProtKB—P10914 (IRF1_HUMAN), or an orthologue thereof, may be mutated with a K to R mutation at either and/or 299 and 275 (highlighted above), (Panda D, Gjinaj E, Bachu M, Squire E, Novatt H, Ozato K, Rabin R L. IRF1 Maintains Optimal Constitutive Expression of Antiviral Genes and Regulates the Early Antiviral Response. Front Immunol. 2019 May 15; 10:1019. doi: 10.3389/fimmu.2019.01019). One embodiment of this mutated IRF1 dominant negative acting polypeptide is represented herein as SEQ ID No:6, as follows:

[SEQ ID No: 6]
GDSSPDTFSDGLSSSTLPDDHSSYTVPGYMQDLEVEQALT
PALSPCAVSSTLPDWHIPVEVVPDSTSDLYNFQVSPMPST
SEATTDEDEEGKLPEDIMKLLEQSEWQPTNVDGKGYLLNE
PGVQPTSVYGDFSCREEPEIDSPGGDIGLSLQRVFTDLRN
MDATWLDSLLTPVRLPSIQAIPCAP

Therefore, preferably the RNA construct of the first aspect comprises a nucleotide sequence which encodes an amino acid sequence substantially as set out in SEQ ID No: 6, or a variant or fragment thereof.

In one embodiment, the mutated IRF1 dominant negative acting polypeptide is encoded by the DNA nucleotide sequence of SEQ ID No: 7 as follows:

[SEQ ID No: 7]
GGGGATTCCAGCCCTGATACCTTCTCTGATGGACTCAGCA
GCTCCACTCTGCCTGATGACCACAGCAGCTACACAGTTCC
AGGCTACATGCAGGACTTGGAGGTGGAGCAGGCCCTGACT
CCAGCACTGTCGCCATGTGCTGTCAGCAGCACTCTCCCCG
ACTGGCACATCCCAGTGGAAGTIGTGCCGGACAGCACCAG
TGATCTGTACAACTTCCAGGTGTCACCCATGCCCTCCACC
TCTGAAGCTACAACAGATGAGGATGAGGAAGGGAAATTAC
CTGAGGACATCATGAAGCTCTTGGAGCAGTCGGAGTGGCA
GCCAACAAACGTGGATGGGAAGGGGTACCTACTCAATGAA
CCTGGAGTCCAGCCCACCTCTGTCTATGGAGACTTTAGCT
GTCGGGAGGAGCCAGAAATTGACAGCCCAGGGGGGGATAT
TGGGCTGAGTCTACAGCGTGTCTTCACAGATCTGQRGAAC
ATGGATGCCACCTGGCTGGACAGCCTGCTGACCCCAGTCC
GGTTGCCCTCCATCCAGGCCATTCCCTGTGCACCG

Accordingly, preferably the mutated IRF1 dominant negative acting polypeptide is encoded by the DNA nucleotide sequence substantially as set out in SEQ ID NO: 7 or a variant or fragment thereof. It will be appreciated that the codons leading to the amino acid changes are highlighted above in bold.

Thus, the RNA construct may comprise an RNA nucleotide sequence of SEQ ID No: 8, as follows:

[SEQ ID No: 8]
GGGGAUUCCAGCCCUGAUACCUUCUCUGAUGGACUCAGCA
GCUCCACUCUGCCUGAUGACCACAGCAGCUACACAGUUCC
AGGCUACAUGCAGGACUUGGAGGUGGAGCAGGCCCUGACU
CCAGCACUGUCGCCAUGUGCUGUCAGCAGCACUCUCCCCG
ACUGGCACAUCCCAGUGGAAGUUGUGCCGGACAGCACCAG
UGAUCUGUACAACUUCCAGGUGUCACCCAUGCCCUCCACC
UCUGAAGCUACAACAGAUGAGGAUGAGGAAGGGAAAUUAC
CUGAGGACAUCAUGAAGCUCUUGGAGCAGUCGGAGUGGCA
GCCAACAAACGUGGAUGGGAAGGGGUACCUACUCAAUGAA
CCUGGAGUCCAGCCCACCUCUGUCUAUGGAGACUUUAGCU
GUCGGGAGGAGCCAGAAAUUGACAGCCCAGGGGGGGAUAU
UGGGCUGAGUCUACAGCGUGUCUUCACAGAUCUGCGGAAC
AUGGAUGCCACCUGGCUGGACAGCCUGCUGACCCCAGUCC
GGUUGCCCUCCAUCCAGGCCAUUCCCUGUGCACCG

Therefore, preferably the RNA construct comprises an RNA nucleotide sequence substantially as set out in SEQ ID No: 8 or a variant or fragment thereof.

The inventors then subjected the protein sequence of SEQ ID No: 6 to codon optimisation for human expression, and one embodiment of the codon optimised nucleic acid (DNA) sequence that includes a start (ATG) and a stop (TGA) codon is provided herein as SEQ ID No: 9 as follows:

[SEQ ID No: 9]
ATGGGCGATAGCAGCCCCGATACCTTTTCCGATGGCCTGA
GCAGCAGCACCCTGCCTGATGATCACAGCAGCTACACCGT
GCCTGGCTACATGCAGGACCTGGAAGTGGAACAGGCCCTG
ACACCAGCTCTGAGCCCTTGTGCTGTGTCCAGCACACTGC
CCGATTGGCACATCCCTGTGGAAGTGGTGCCTGACAGCAC
CAGCGACCTGTACAACTTCCAAGTGTCCCCTATGCCTAGC
ACCTCCGAGGCCACCACCGATGAGGATGAAGAGGGAAAGC
TGCCCGAGGACATCATGAAGCTGCTGGAACAGAGCGAGTG
GCAGCCCACCAATGTGGATGGCAAGGGCTACCTGCTGAAC
GAGCCTGGCGTTCAGCCTACAAGCGTGTACGGCGACTTCA
GCTGCAGAGAGGAACCCGAGATCGATAGCCCTGGCGGCGA
TATCGGACTGAGTCTGCAGAGGGTGTTCACCGACCTGAGA
AACATGGACGCCACCTGGCTGGACAGCCTGCTGACACCTG
TTAGACTGCCCTCTATCCAGGCTATCCCCTGCGCTCCTTG
A

Hence, preferably the RNA construct is encoded by a DNA sequence substantially as set out in SEQ ID No: 9 or a fragment or variant thereof.

In an embodiment, the RNA sequence corresponding to the codon optimised DNA sequence of SEQ ID No: 9 that includes a start (AUG) and a stop (UGA) codon is provided herein as SEQ ID No: 10 as follows:

[SEQ ID No: 10]
AUGGGCGAUAGCAGCCCCGAUACCUUUUCCGAUGGCCUGA
GCAGCAGCACCCUGCCUGAUGAUCACAGCAGCUACACCGU
GCCUGGCUACAUGCAGGACCUGGAAGUGGAACAGGCCCUG
ACACCAGCUCUGAGCCCUUGUGCUGUGUCCAGCACACUGC
CCGAUUGGCACAUCCCUGUGGAAGUGGUGCCUGACAGCAC
CAGCGACCUGUACAACUUCCAAGUGUCCCCUAUGCCUAGC
ACCUCCGAGGCCACCACCGAUGAGGAUGAAGAGGGAAAGC
UGCCCGAGGACAUCAUGAAGCUGCUGGAACAGAGCGAGUG
GCAGCCCACCAAUGUGGAUGGCAAGGGCUACCUGCUGAAC
GAGCCUGGCGUUCAGCCUACAAGCGUGUACGGCGACUUCA
GCUGCAGAGAGGAACCCGAGAUCGAUAGCCCUGGCGGCGA
UAUCGGACUGAGUCUGCAGAGGGUGUUCACCGACCUGAGA
AACAUGGACGCCACCUGGCUGGACAGCCUGCUGACACCUG
UUAGACUGCCCUCUAUCCAGGCUAUCCCCUGCGCUCCUUG
A

Hence, preferably the RNA construct comprises a sequence substantially as set out in SEQ ID No: 10 or a fragment or variant thereof.

In one embodiment, the at least one IMP may be an IRF3 dominant negative acting form, which is also key to IFN induction cascade, i.e. a dominant negative acting version of IRF3 with the DBD deleted, IRF3 (191-427) (NCBI Reference Sequence: NM_001571.6; UniProtKB—Q14653 (IRF3_HUMAN)), or an orthologue thereof—Ysebrant de Lendonck L, Martinet V, Goriely S. Interferon regulatory factor 3 in adaptive immune responses. Cell Mol Life Sci. 2014 October; 71(20):3873-83. doi: 10.1007/s00018-014-1653-9. One embodiment of this IRF3 dominant negative acting form is represented herein as SEQ ID No: 11, as follows:

[SEQ ID No: 11]
PLKRLLVPGEEWEFEVTAFYRGRQVFQQTISCPEGLRLVGSEVGDRTLPGWPVTLPDPGMSLTDRGVMSYVRHVLSCL
GGGLALWRAGQWLWAQRLGHCHTYWAVSEELLPNSGHGPDGEVPKDKEGGVFDLGPFIVDLITFTEGSGRSPRYALWF
CVGESWPQDQPWTKRLVMVKVVPTCLRALVEMARVGGASSLENTVDLHISNSHPLSLTSDQYKAYLQDLVEGMDFQGP
GES

Therefore, preferably the RNA construct of the first aspect comprises a nucleotide sequence which encodes an amino acid sequence substantially as set out in SEQ ID No: 11 or a variant or fragment thereof.

In one embodiment, the IRF3 dominant negative acting form polypeptide is encoded by the DNA nucleotide sequence of SEQ ID No: 12, as follows:

[SEQ ID No: 12]
CCACTGAAGCGGCTGTTGGTGCCGGGGGAAGAGTGGGAGTTCGAGGTGACAGCCTTCTACCGGGGCCGCCAAGTCTTC
CAGCAGACCATCTCCTGCCCGGAGGGCCTGCGGCTGGTGGGGTCCGAAGTGGGAGACAGGACGCTGCCTGGATGGCCA
GTCACACTGCCAGACCCTGGCATGTCCCTGACAGACAGGGGAGTGATGAGCTACGTGAGGCATGTGCTGAGCTGCCTG
GGTGGGGGACTGGCTCTCTGGCGGGCCGGGCAGTGGCTCTGGGCCCAGCGGCTGGGGCACTGCCACACATACTGGGCA
GTGAGCGAGGAGCTGCTCCCCAACAGCGGGCATGGGCCTGATGGCGAGGTCCCCAAGGACAAGGAAGGAGGCGTGTTT
GACCTGGGGCCCTTCATTGTAGATCTGATTACCTTCACGGAAGGAAGCGGACGCTCACCACGCTATGCCCTCTGGTTC
TGTGTGGGGGAGTCATGGCCCCAGGACCAGCCGTGGACCAAGAGGCTCGTGATGGTCAAGGTTGTGCCCACGTGCCTC
AGGGCCTTGGTAGAAATGGCCCGGGTAGGGGGTGCCTCCTCCCTGGAGAATACTGTGGACCTGCACATTTCCAACAGC
CACCCACTCTCCCTCACCTCCGACCAGTACAAGGCCTACCTGCAGGACTTGGTGGAGGGCATGGATTTCCAGGGCCCT
GGGGAGAGC

Accordingly, preferably the IRF3 dominant negative acting form polypeptide is encoded by the DNA nucleotide sequence substantially as set out in SEQ ID NO: 12 or a variant or fragment thereof.

Thus, the RNA construct may comprise an RNA nucleotide sequence of SEQ ID No: 13 as follows:

[SEQ ID No: 13]
CCACUGAAGCGGCUGUUGGUGCCGGGGGAAGAGUGGGAGUUCGAGGUGACAGCCUUCUACCGGGGCCGCCAAGUCUUC
CAGCAGACCAUCUCCUGCCCGGAGGGCCUGCGGCUGGUGGGGUCCGAAGUGGGAGACAGGACGCUGCCUGGAUGGCCA
GUCACACUGCCAGACCCUGGCAUGUCCCUGACAGACAGGGGAGUGAUGAGCUACGUGAGGCAUGUGCUGAGCUGCCUG
GGUGGGGGACUGGCUCUCUGGCGGGCCGGGCAGUGGCUCUGGGCCCAGCGGCUGGGGCACUGCCACACAUACUGGGCA
GUGAGCGAGGAGCUGCUCCCCAACAGCGGGCAUGGGCCUGAUGGCGAGGUCCCCAAGGACAAGGAAGGAGGCGUGUUU
GACCUGGGGCCCUUCAUUGUAGAUCUGAUUACCUUCACGGAAGGAAGCGGACGCUCACCACGCUAUGCCCUCUGGUUC
UGUGUGGGGGAGUCAUGGCCCCAGGACCAGCCGUGGACCAAGAGGCUCGUGAUGGUCAAGGUUGUGCCCACGUGCCUC
AGGGCCUUGGUAGAAAUGGCCCGGGUAGGGGGUGCCUCCUCCCUGGAGAAUACUGUGGACCUGCACAUUUCCAACAGC
CACCCACUCUCCCUCACCUCCGACCAGUACAAGGCCUACCUGCAGGACUUGGUGGAGGGCAUGGAUUUCCAGGGCCCU
GGGGAGAGC

Therefore, preferably the RNA construct comprises an RNA nucleotide sequence substantially as set out in SEQ ID No: 13 or a variant or fragment thereof.

The inventors then subjected the protein sequence of SEQ ID No: 11 to codon optimisation for human expression, and one embodiment of the codon optimised nucleic acid (DNA) sequence that includes a start (ATG) and a stop (TGA) codon is provided herein as SEQ ID No: 14 as follows:

[SEQ ID No: 14]
ATGCCCCTGAAGAGACTGCTGGTGCCTGGCGAGGAATGGGAGTTTGAAGTGACCGCCTTCTACCGGGGCAGACAGGTG
TTCCAGCAGACCATCTCTTGCCCCGAGGGACTGAGACTCGTGGGCTCTGAAGTGGGCGATAGAACACTGCCTGGCTGG
CCTGTGACACTGCCAGATCCTGGAATGAGCCTGACCGACAGAGGCGTGATGAGCTATGTGCGGCACGTGCTGTCTTGT
CTCGGCGGAGGACTTGCCCTTTGGAGAGCTGGACAATGGCTGTGGGCTCAGAGACTGGGCCACTGTCACACATACTGG
GCCGTGTCTGAGGAACTGCTGCCCAATTCTGGCCACGGACCTGATGGCGAGGTGCCCAAAGACAAAGAAGGCGGCGTT
TTCGATCTGGGCCCCTTCATCGTGGACCTGATCACCTTTACCGAAGGCAGCGGCAGAAGCCCCAGATACGCCCTGTGG
TTTTGTGTGGGCGAGAGCTGGCCTCAGGATCAGCCTTGGACCAAGAGACTGGTCATGGTCAAGGTGGTGCCTACCTGC
CTGAGAGCCCTGGTGGAAATGGCTAGAGTTGGCGGAGCCAGCAGCCTGGAAAACACCGTGGATCTGCACATCAGCAAC
TCTCACCCTCTGTCTCTGACCAGCGACCAGTACAAGGCCTATCTGCAGGACCTGGTCGAAGGCATGGACTTTCAAGGC
CCTGGCGAGTCCTGA

Hence, preferably the RNA construct is encoded by a DNA sequence substantially as set out in SEQ ID No: 14 or a fragment or variant thereof.

In an embodiment, the RNA sequence corresponding to the codon optimised DNA sequence of SEQ ID No: 14 that includes a start (AUG) and a stop (UGA) codon is provided herein as SEQ ID No: 15, as follows:

[SEQ ID No: 15]
AUGCCCCUGAAGAGACUGCUGGUGCCUGGCGAGGAAUGGGAGUUUGAAGUGACCGCCUUCUACCGGGGCAGACAGGUG
UUCCAGCAGACCAUCUCUUGCCCCGAGGGACUGAGACUCGUGGGCUCUGAAGUGGGCGAUAGAACACUGCCUGGCUGG
CCUGUGACACUGCCAGAUCCUGGAAUGAGCCUGACCGACAGAGGCGUGAUGAGCUAUGUGCGGCACGUGCUGUCUUGU
CUCGGCGGAGGACUUGCCCUUUGGAGAGCUGGACAAUGGCUGUGGGCUCAGAGACUGGGCCACUGUCACACAUACUGG
GCCGUGUCUGAGGAACUGCUGCCCAAUUCUGGCCACGGACCUGAUGGCGAGGUGCCCAAAGACAAAGAAGGCGGCGUU
UUCGAUCUGGGCCCCUUCAUCGUGGACCUGAUCACCUUUACCGAAGGCAGCGGCAGAAGCCCCAGAUACGCCCUGUGG
UUUUGUGUGGGCGAGAGCUGGCCUCAGGAUCAGCCUUGGACCAAGAGACUGGUCAUGGUCAAGGUGGUGCCUACCUGC
CUGAGAGCCCUGGUGGAAAUGGCUAGAGUUGGCGGAGCCAGCAGCCUGGAAAACACCGUGGAUCUGCACAUCAGCAAC
UCUCACCCUCUGUCUCUGACCAGCGACCAGUACAAGGCCUAUCUGCAGGACCUGGUCGAAGGCAUGGACUUUCAAGGC
CCUGGCGAGUCCUGA

Hence, preferably the RNA construct comprises a sequence substantially as set out in SEQ ID No: 15, or a fragment or variant thereof.

In one embodiment, the at least one IMP may be an IRF7 dominant negative acting form, which is also key to the IFN induction cascade, and which impacts on IFN alpha and beta induction (NCBI Reference Sequence: NM_001572.5; UniProtKB—Q92985 (IRF7_HUMAN)), or an orthologue thereof (Au W C, Yeow W S, Pitha P M. Analysis of functional domains of interferon regulatory factor 7 and its association with IRF-3. Virology. 2001; 280(2):273-282. doi:10.1006/vir0.2000.0782). One embodiment of this IRF7 dominant negative acting form is referred to as IRF-7 (238-503), and is represented herein as SEQ ID No: 16, as follows:

[SEQ ID No: 16]
WAVETTPSPGPQPAALTTGEAAAPESPHQAEPYLSPSPSACTAVQEPSPGALDVTIMYKGRTVLQKVVGHPSCTFLYG
PPDPAVRATDPQQVAFPSPAELPDQKQLRYTEELLRHVAPGLHLELRGPQLWARRMGKCKVYWEVGGPPGSASPSTPA
CLLPRNCDTPIFDFRVFFQELVEFRARQRRGSPRYTIYLGFGQDLSAGRPKEKSLVLVKLEPWLCRVHLEGTQREGVS
SLDSSSLSLCLSSANSLYDDIECFLMELEQPA

Therefore, preferably the RNA construct of the first aspect comprises a nucleotide sequence which encodes an amino acid sequence substantially as set out in SEQ ID No: 16, or a variant or fragment thereof.

In one embodiment, the IRF7 dominant negative acting form polypeptide is encoded by the DNA nucleotide sequence of SEQ ID No: 17, as follows:

[SEQ ID No: 17]
TGGGCAGTAGAGACGACCCCCAGCCCCGGGCCCCAGCCCGCGGCACTAACGACAGGCGAGGCCGCGGCCCCAGAGTCC
CCGCACCAGGCAGAGCCGTACCTGTCACCCTCCCCAAGCGCCTGCACCGCGGTGCAAGAGCCCAGCCCAGGGGCGCTG
GACGTGACCATCATGTACAAGGGCCGCACGGTGCTGCAGAAGGTGGTGGGACACCCGAGCTGCACGTTCCTATACGGC
CCCCCAGACCCAGCTGTCCGGGCCACAGACCCCCAGCAGGTAGCATTCCCCAGCCCTGCCGAGCTCCCGGACCAGAAG
CAGCTGCGCTACACGGAGGAACTGCTGCGGCACGTGGCCCCTGGGTTGCACCTGGAGCTTCGGGGGCCACAGCTGTGG
GCCCGGCGCATGGGCAAGTGCAAGGTGTACTGGGAGGTGGGCGGACCCCCAGGCTCCGCCAGCCCCTCCACCCCAGCC
TGCCTGCTGCCTCGGAACTGTGACACCCCCATCTTCGACTTCAGAGTCTTCTTCCAAGAGCTGGTGGAATTCCGGGCA
CGGCAGCGCCGTGGCTCCCCACGCTATACCATCTACCTGGGCTTCGGGCAGGACCTGTCAGCTGGGAGGCCCAAGGAG
AAGAGCCTGGTCCTGGTGAAGCTGGAACCCTGGCTGTGCCGAGTGCACCTAGAGGGCACGCAGCGTGAGGGTGTGTCT
TCCCTGGATAGCAGCAGCCTCAGCCTCTGCCTGTCCAGCGCCAACAGCCTCTATGACGACATCGAGTGCTTCCTTATG
GAGCTGGAGCAGCCCGCC

Accordingly, preferably the IRF7 dominant negative acting form polypeptide is encoded by the DNA nucleotide sequence substantially as set out in SEQ ID NO: 17, or a variant or fragment thereof.

Furthermore, preferably the RNA construct of the first aspect comprises an RNA nucleotide sequence substantially as set out in SEQ ID No: 18, or a variant or fragment thereof.

[SEQ ID No: 18]
UGGGCAGUAGAGACGACCCCCAGCCCCGGGCCCCAGCCCGCGGCACUAACGACAGGCGAGGCCGCGGCCCCAGAGUCC
CCGCACCAGGCAGAGCCGUACCUGUCACCCUCCCCAAGCGCCUGCACCGCGGUGCAAGAGCCCAGCCCAGGGGCGCUG
GACGUGACCAUCAUGUACAAGGGCCGCACGGUGCUGCAGAAGGUGGUGGGACACCCGAGCUGCACGUUCCUAUACGGC
CCCCCAGACCCAGCUGUCCGGGCCACAGACCCCCAGCAGGUAGCAUUCCCCAGCCCUGCCGAGCUCCCGGACCAGAAG
CAGCUGCGCUACACGGAGGAACUGCUGCGGCACGUGGCCCCUGGGUUGCACCUGGAGCUUCGGGGGCCACAGCUGUGG
GCCCGGCGCAUGGGCAAGUGCAAGGUGUACUGGGAGGUGGGCGGACCCCCAGGCUCCGCCAGCCCCUCCACCCCAGCC
UGCCUGCUGCCUCGGAACUGUGACACCCCCAUCUUCGACUUCAGAGUCUUCUUCCAAGAGCUGGUGGAAUUCCGGGCA
CGGCAGCGCCGUGGCUCCCCACGCUAUACCAUCUACCUGGGCUUCGGGCAGGACCUGUCAGCUGGGAGGCCCAAGGAG
AAGAGCCUGGUCCUGGUGAAGCUGGAACCCUGGCUGUGCCGAGUGCACCUAGAGGGCACGCAGCGUGAGGGUGUGUCU
UCCCUGGAUAGCAGCAGCCUCAGCCUCUGCCUGUCCAGCGCCAACAGCCUCUAUGACGACAUCGAGUGCUUCCUUAUG
GAGCUGGAGCAGCCCGCC

The inventors then subjected the protein sequence of SEQ ID No: 16 to codon optimisation for human expression, and one embodiment of the codon optimised nucleic acid (DNA) sequence that includes a start (ATG) and a stop (TGA) codon is provided herein as SEQ ID No: 19, as follows:

[SEQ ID No: 19]
ATGTGGGCCGTCGAGACAACACCTTCTCCAGGACCTCAACCTGCCGCTCTGACAACAGGCGAAGCTGCTGCTCCTGAG
TCTCCACATCAGGCCGAGCCTTACCTGTCTCCATCTCCTAGCGCCTGTACCGCCGTGCAAGAACCTTCTCCTGGTGCT
CTGGACGTGACCATCATGTACAAGGGCAGAACCGTGCTGCAGAAAGTCGTGGGACACCCCAGCTGCACCTTTCTGTAT
GGCCCTCCAGATCCTGCCGTGCGGGCTACAGATCCTCAGCAGGTTGCATTCCCATCTCCAGCCGAGCTGCCCGATCAG
AAGCAGCTGAGATACACCGAGGAACTGCTGAGACACGTGGCCCCTGGACTGCACCTGGAACTGAGAGGACCACAACTG
TGGGCCAGACGGATGGGCAAGTGCAAGGTGTACTGGGAAGTTGGCGGCCCTCCTGGATCTGCCTCTCCATCTACACCA
GCCTGCCTGCTGCCTCGGAATTGCGACACCCCTATCTTCGACTTCCGGGTGTTCTTCCAAGAGCTGGTGGAATTCCGG
GCCAGACAGAGAAGAGGCAGCCCCAGATACACCATCTACCTCGGCTTTGGCCAGGACCTGTCTGCCGGACGGCCTAAA
GAAAAGTCCCTGGTGCTGGTCAAGCTGGAACCCTGGCTGTGTAGAGTGCATCTGGAAGGCACCCAGAGAGAGGGCGTC
AGCAGCCTGGATAGCAGCTCTCTGAGCCTGTGTCTGAGCAGCGCCAACAGCCTGTACGACGATATCGAGTGCTTCCTG
ATGGAACTGGAACAGCCCGCCTGA

Hence, preferably the RNA construct is encoded by a DNA sequence substantially as set out in SEQ ID No: 19, or a fragment or variant thereof.

In an embodiment, the RNA sequence corresponding to the codon optimised DNA sequence of SEQ ID No: 19 that includes a start (AUG) and a stop (UGA) codon is provided herein as SEQ ID No: 20, as follows:

[SEQ ID No: 20]
AUGUGGGCCGUCGAGACAACACCUUCUCCAGGACCUCAACCUGCCGCUCUGACAACAGGCGAAGCUGCUGCUCCUGAG
UCUCCACAUCAGGCCGAGCCUUACCUGUCUCCAUCUCCUAGCGCCUGUACCGCCGUGCAAGAACCUUCUCCUGGUGCU
CUGGACGUGACCAUCAUGUACAAGGGCAGAACCGUGCUGCAGAAAGUCGUGGGACACCCCAGCUGCACCUUUCUGUAU
GGCCCUCCAGAUCCUGCCGUGCGGGCUACAGAUCCUCAGCAGGUUGCAUUCCCAUCUCCAGCCGAGCUGCCCGAUCAG
AAGCAGCUGAGAUACACCGAGGAACUGCUGAGACACGUGGCCCCUGGACUGCACCUGGAACUGAGAGGACCACAACUG
UGGGCCAGACGGAUGGGCAAGUGCAAGGUGUACUGGGAAGUUGGCGGCCCUCCUGGAUCUGCCUCUCCAUCUACACCA
GCCUGCCUGCUGCCUCGGAAUUGCGACACCCCUAUCUUCGACUUCCGGGUGUUCUUCCAAGAGCUGGUGGAAUUCCGG
GCCAGACAGAGAAGAGGCAGCCCCAGAUACACCAUCUACCUCGGCUUUGGCCAGGACCUGUCUGCCGGACGGCCUAAA
GAAAAGUCCCUGGUGCUGGUCAAGCUGGAACCCUGGCUGUGUAGAGUGCAUCUGGAAGGCACCCAGAGAGAGGGCGUC
AGCAGCCUGGAUAGCAGCUCUCUGAGCCUGUGUCUGAGCAGCGCCAACAGCCUGUACGACGAUAUCGAGUGCUUCCUG
AUGGAACUGGAACAGCCCGCCUGA

Hence, preferably the RNA construct comprises a sequence substantially as set out in SEQ ID No: 20, or a fragment or variant thereof.

In one embodiment, the at least one IMP may be an IRF9 dominant negative acting form, IRF9 (142-393) (NCBI Reference Sequence: NM_006084.5; UniProtKB—Q00978 (IRF9_HUMAN)), or an orthologue thereof—(Paul A, Tang T H, Ng S K. Interferon Regulatory Factor 9 Structure and Regulation. Front Immunol. 2018 Aug. 10; 9:1831. doi: 10.3389/fimmu.2018.01831. PMID: 30147694; PMCID: PMC6095977.).

One embodiment of this IRF9 dominant negative acting form is represented herein as SEQ ID No: 21, as follows:

[SEQ ID No: 21]
RKEEEDAMQNCTLSPSVLQDSLNNEEEGASGGAVHSDIGSSSSSSSPEPQEVTDTTEAPFQGDQRSLEFLLPPEPDYS
LLLTFIYNGRVVGEAQVQSLDCRLVAEPSGSESSMEQVLFPKPGPLEPTQRLLSQLERGILVASNPRGLFVQRLCPIP
ISWNAPQAPPGPGPHLLPSNECVELFRTAYFCRDLVRYFQGLGPPPKFQVTLNFWEESHGSSHTPQNLITVKMEQAFA
RYLLEQTPEQQAAILSLV

Therefore, preferably the RNA construct of the first aspect comprises a nucleotide sequence which encodes an amino acid sequence substantially as set out in SEQ ID No: 21, or a variant or fragment thereof.

Therefore, preferably the RNA construct of the first aspect comprises a DNA nucleotide sequence substantially as set out in SEQ ID No: 22, or a variant or fragment thereof.

[SEQ ID No: 22]
AAGGAGGAAGAGGATGCCATGCAGAACTGCACACTCAGTCCCTCTGTGCTCCAGGACTCCCTCAATAATGAGGAGGAG
GGGGCCAGTGGGGGAGCAGTCCATTCAGACATTGGGAGCAGCAGCAGCAGCAGCAGCCCTGAGCCACAGGAAGTTACA
GACACAACTGAGGCCCCCTTTCAAGGGGATCAGAGGTCCCTGGAGTTTCTGCTTCCTCCAGAGCCAGACTACTCACTG
CTGCTCACCTTCATCTACAACGGGCGCGTGGTGGGCGAGGCCCAGGTGCAAAGCCTGGATTGCCGCCTTGTGGCTGAG
CCCTCAGGCTCTGAGAGCAGCATGGAGCAGGTGCTGTTCCCCAAGCCTGGCCCACTGGAGCCCACGCAGCGCCTGCTG
AGCCAGCTTGAGAGGGGCATCCTAGTGGCCAGCAACCCCCGAGGCCTCTTCGTGCAGCGCCTTTGCCCCATCCCCATC
TCCTGGAATGCACCCCAGGCTCCACCTGGGCCAGGCCCGCATCTGCTGCCCAGCAACGAGTGCGTGGAGCTCTTCAGA
ACCGCCTACTTCTGCAGAGACTTGGTCAGGTACTTTCAGGGCCTGGGCCCCCCACCGAAGTTCCAGGTAACACTGAAT
TTCTGGGAAGAGAGCCATGGCTCCAGCCATACTCCACAGAATCTTATCACAGTGAAGATGGAGCAGGCCTTTGCCCGA
TACTTGCTGGAGCAGACTCCAGAGCAGCAGGCAGCCATTCTGTCCCTGGTG

Accordingly, preferably the IRF9 dominant negative acting form polypeptide is encoded by the DNA nucleotide sequence substantially as set out in SEQ ID NO: 22, or a variant or fragment thereof.

Thus, the RNA construct may comprise an RNA nucleotide sequence of SEQ ID No: 23, as follows:

[SEQ ID No: 23]
AAGGAGGAAGAGGAUGCCAUGCAGAACUGCACACUCAGUCCCUCUGUGCUCCAGGACUCCCUCAAUAAUGAGGAGGAG
GGGGCCAGUGGGGGAGCAGUCCAUUCAGACAUUGGGAGCAGCAGCAGCAGCAGCAGCCCUGAGCCACAGGAAGUUACA
GACACAACUGAGGCCCCCUUUCAAGGGGAUCAGAGGUCCCUGGAGUUUCUGCUUCCUCCAGAGCCAGACUACUCACUG
CUGCUCACCUUCAUCUACAACGGGCGCGUGGUGGGCGAGGCCCAGGUGCAAAGCCUGGAUUGCCGCCUUGUGGCUGAG
CCCUCAGGCUCUGAGAGCAGCAUGGAGCAGGUGCUGUUCCCCAAGCCUGGCCCACUGGAGCCCACGCAGCGCCUGCUG
AGCCAGCUUGAGAGGGGCAUCCUAGUGGCCAGCAACCCCCGAGGCCUCUUCGUGCAGCGCCUUUGCCCCAUCCCCAUC
UCCUGGAAUGCACCCCAGGCUCCACCUGGGCCAGGCCCGCAUCUGCUGCCCAGCAACGAGUGCGUGGAGCUCUUCAGA
ACCGCCUACUUCUGCAGAGACUUGGUCAGGUACUUUCAGGGCCUGGGCCCCCCACCGAAGUUCCAGGUAACACUGAAU
UUCUGGGAAGAGAGCCAUGGCUCCAGCCAUACUCCACAGAAUCUUAUCACAGUGAAGAUGGAGCAGGCCUUUGCCCGA
UACUUGCUGGAGCAGACUCCAGAGCAGCAGGCAGCCAUUCUGUCCCUGGUG

Therefore, preferably the RNA construct comprises an RNA nucleotide sequence substantially as set out in SEQ ID No: 23, or a variant or fragment thereof.

The inventors then subjected the protein sequence of SEQ ID No: 21 to codon optimisation for human expression, and one embodiment of the codon optimised nucleic acid (DNA) sequence that includes a start (ATG) and a stop (TGA) codon is provided herein as SEQ ID No: 24, as follows:

[SEQ ID No: 24]
ATGAAGGAAGAAGAGGACGCCATGCAGAACTGCACACTGAGCCCAAGCGTGCTGCAGGACAGCCTGAACAATGAGGAA
GAAGGCGCCTCTGGCGGAGCCGTGCACTCTGATATTGGCAGCAGCAGCTCTAGCAGCAGCCCCGAGCCTCAAGAAGTG
ACCGATACAACAGAGGCCCCATTCCAGGGCGACCAGCGGAGTCTGGAATTTCTGCTGCCTCCTGAGCCTGACTACAGC
CTGCTGCTGACCTTCATCTACAACGGCAGAGTCGTGGGCGAAGCCCAGGTGCAGTCTCTGGATTGCAGACTGGTGGCC
GAGCCTAGCGGAAGCGAGTCTAGTATGGAACAGGTGCTGTTCCCCAAGCCTGGACCTCTGGAACCCACACAGAGGCTG
CTGTCTCAACTGGAAAGGGGCATCCTGGTGGCCAGCAATCCTAGAGGCCTGTTCGTGCAGAGACTGTGCCCTATTCCT
ATCAGCTGGAACGCCCCTCAGGCTCCTCCTGGACCTGGACCACATCTGCTGCCCAGCAATGAGTGCGTGGAACTGTTC
CGGACCGCCTACTTCTGCAGAGATCTCGTGCGGTACTTCCAAGGCCTGGGACCTCCTCCAAAGTTCCAAGTGACCCTG
AACTTCTGGGAAGAGAGCCACGGCAGCAGCCACACACCTCAGAATCTGATCACCGTGAAGATGGAACAAGCCTTCGCC
AGATACCTGCTGGAACAGACCCCTGAACAGCAGGCCGCCATCCTGTCTCTGGTGTGA

Hence, preferably the RNA construct is encoded by a DNA sequence substantially as set out in SEQ ID No: 24, or a fragment or variant thereof.

In an embodiment, the RNA sequence corresponding to the codon optimised DNA sequence of SEQ ID No: 24 that includes a start (AUG) and a stop (UGA) codon is provided herein as SEQ ID No: 25, as follows:

[SEQ ID No: 25]
AUGAAGGAAGAAGAGGACGCCAUGCAGAACUGCACACUGAGCCCAAGCGUGCUGCAGGACAGCCUGAACAAUGAGGAA
GAAGGCGCCUCUGGCGGAGCCGUGCACUCUGAUAUUGGCAGCAGCAGCUCUAGCAGCAGCCCCGAGCCUCAAGAAGUG
ACCGAUACAACAGAGGCCCCAUUCCAGGGCGACCAGCGGAGUCUGGAAUUUCUGCUGCCUCCUGAGCCUGACUACAGC
CUGCUGCUGACCUUCAUCUACAACGGCAGAGUCGUGGGCGAAGCCCAGGUGCAGUCUCUGGAUUGCAGACUGGUGGCC
GAGCCUAGCGGAAGCGAGUCUAGUAUGGAACAGGUGCUGUUCCCCAAGCCUGGACCUCUGGAACCCACACAGAGGCUG
CUGUCUCAACUGGAAAGGGGCAUCCUGGUGGCCAGCAAUCCUAGAGGCCUGUUCGUGCAGAGACUGUGCCCUAUUCCU
AUCAGCUGGAACGCCCCUCAGGCUCCUCCUGGACCUGGACCACAUCUGCUGCCCAGCAAUGAGUGCGUGGAACUGUUC
CGGACCGCCUACUUCUGCAGAGAUCUCGUGCGGUACUUCCAAGGCCUGGGACCUCCUCCAAAGUUCCAAGUGACCCUG
AACUUCUGGGAAGAGAGCCACGGCAGCAGCCACACACCUCAGAAUCUGAUCACCGUGAAGAUGGAACAAGCCUUCGCC
AGAUACCUGCUGGAACAGACCCCUGAACAGCAGGCCGCCAUCCUGUCUCUGGUGUGA

Hence, preferably the RNA construct comprises a sequence substantially as set out in SEQ ID No: 25, or a fragment or variant thereof.

In another embodiment, the IRF9 dominant negative acting form of SEQ ID No:21 may be mutated by reducing it down to amino acid residues 182-385 of SEQ ID No:26 or a fragment or variant thereof (NCBI Reference Sequence: NM_006084.5; UniProtKB—Q00978 (IRF9_HUMAN)), or an orthologue thereof.

[SEQ ID No: 26]
SSSSSSPEPQEVTDTTEAPFQGDORSLEFLLPPEPDYSLLLTFIYNGRVVGEAQVQSLDCRLVAEPSGSESSMEQVLF
PKPGPLEPTQRLLSQLERGILVASNPRGLFVQRLCPIPISWNAPQAPPGPGPHLLPSNECVELFRTAYFCRDLVRYFQ
GLGPPPKFQVTLNFWEESHGSSHTPQNLITVKMEQAFARYLLEQTPEQ

Therefore, preferably the RNA construct of the first aspect comprises a DNA nucleotide sequence which encodes an amino acid sequence substantially as set out in SEQ ID No: 26, or a variant or fragment thereof.

In one embodiment, the mutated IRF9 dominant negative acting form polypeptide (IRF9 (182-235)) is encoded by the DNA nucleotide sequence of SEQ ID No: 27, as follows:

[SEQ ID No: 27]
AGCAGCAGCAGCAGCAGCCCTGAGCCACAGGAAGTTACAGACACAACTGAGGCCCCCTTTCAAGGGGATCAGAGGTCC
CTGGAGTTTCTGCTTCCTCCAGAGCCAGACTACTCACTGCTGCTCACCTTCATCTACAACGGGCGCGTGGTGGGCGAG
GCCCAGGTGCAAAGCCTGGATTGCCGCCTTGTGGCTGAGCCCTCAGGCTCTGAGAGCAGCATGGAGCAGGTGCTGTTC
CCCAAGCCTGGCCCACTGGAGCCCACGCAGCGCCTGCTGAGCCAGCTTGAGAGGGGCATCCTAGTGGCCAGCAACCCC
CGAGGCCTCTTCGTGCAGCGCCTTTGCCCCATCCCCATCTCCTGGAATGCACCCCAGGCTCCACCTGGGCCAGGCCCG
CATCTGCTGCCCAGCAACGAGTGCGTGGAGCTCTTCAGAACCGCCTACTTCTGCAGAGACTTGGTCAGGTACTTTCAG
GGCCTGGGCCCCCCACCGAAGTTCCAGGTAACACTGAATTTCTGGGAAGAGAGCCATGGCTCCAGCCATACTCCACAG
AATCTTATCACAGTGAAGATGGAGCAGGCCTTTGCCCGATACTTGCTGGAGCAGACTCCAGAGCAG

Accordingly, preferably the mutated IRF9 dominant negative acting form is encoded by the DNA nucleotide sequence substantially as set out in SEQ ID NO: 27, or a variant or fragment thereof.

Thus, the RNA construct may comprise an RNA nucleotide sequence of SEQ ID No: 28, as follows:

[SEQ ID No: 28]
AGCAGCAGCAGCAGCAGCCCUGAGCCACAGGAAGUUACAGACACAACUGAGGCCCCCUUUCAAGGGGAUCAGAGGUCC
CUGGAGUUUCUGCUUCCUCCAGAGCCAGACUACUCACUGCUGCUCACCUUCAUCUACAACGGGCGCGUGGUGGGCGAG
GCCCAGGUGCAAAGCCUGGAUUGCCGCCUUGUGGCUGAGCCCUCAGGCUCUGAGAGCAGCAUGGAGCAGGUGCUGUUC
CCCAAGCCUGGCCCACUGGAGCCCACGCAGCGCCUGCUGAGCCAGCUUGAGAGGGGCAUCCUAGUGGCCAGCAACCCC
CGAGGCCUCUUCGUGCAGCGCCUUUGCCCCAUCCCCAUCUCCUGGAAUGCACCCCAGGCUCCACCUGGGCCAGGCCCG
CAUCUGCUGCCCAGCAACGAGUGCGUGGAGCUCUUCAGAACCGCCUACUUCUGCAGAGACUUGGUCAGGUACUUUCAG
GGCCUGGGCCCCCCACCGAAGUUCCAGGUAACACUGAAUUUCUGGGAAGAGAGCCAUGGCUCCAGCCAUACUCCACAG
AAUCUUAUCACAGUGAAGAUGGAGCAGGCCUUUGCCCGAUACUUGCUGGAGCAGACUCCAGAGCAG

Therefore, preferably the RNA construct comprises an RNA nucleotide sequence substantially as set out in SEQ ID No: 28, or a variant or fragment thereof.

The inventors then subjected the protein sequence of SEQ ID No: 26 to codon optimisation for human expression, and one embodiment of the codon optimised nucleic acid (DNA) sequence that includes a start (ATG) and a stop (TGA) codon is provided herein as SEQ ID No: 29, as follows:

[SEQ ID No: 29]
ATGAGCAGCTCTAGCAGCAGCCCCGAGCCTCAAGAAGTGACCGATACAACAGAGGCCCCATTCCAGGGCGACCAGCGG
AGTCTGGAATTTCTGCTGCCTCCTGAGCCTGACTACAGCCTGCTGCTGACCTTCATCTACAACGGCAGAGTCGTGGGC
GAAGCCCAGGTGCAGTCTCTGGATTGCAGACTGGTGGCCGAGCCTAGCGGAAGCGAGTCTAGTATGGAACAGGTGCTG
TTCCCCAAGCCTGGACCTCTGGAACCCACACAGAGGCTGCTGTCTCAACTGGAAAGGGGCATCCTGGTGGCCAGCAAT
CCTAGAGGCCTGTTCGTGCAGAGACTGTGCCCTATTCCTATCAGCTGGAACGCCCCTCAGGCTCCTCCTGGACCTGGA
CCACATCTGCTGCCCAGCAATGAGTGCGTGGAACTGTTCCGGACCGCCTACTTCTGCAGAGATCTCGTGCGGTACTTC
CAAGGCCTGGGACCTCCTCCAAAGTTCCAAGTGACCCTGAACTTCTGGGAAGAGAGCCACGGCAGCAGCCACACACCT
CAGAATCTGATCACCGTGAAGATGGAACAAGCCTTCGCCAGATACCTGCTGGAACAGACCCCTGAACAGTGA

Hence, preferably the RNA construct is encoded by a DNA sequence substantially as set out in SEQ ID No: 29, or a fragment or variant thereof.

In an embodiment, the RNA sequence corresponding to the codon optimised DNA sequence of SEQ ID No: 29 that includes a start (AUG) and a stop (UGA) codon is provided herein as SEQ ID No: 30, as follows:

[SEQ ID No: 30]
AUGAGCAGCUCUAGCAGCAGCCCCGAGCCUCAAGAAGUGACCGAUACAACAGAGGCCCCAUUCCAGGGCGACCAGCGG
AGUCUGGAAUUUCUGCUGCCUCCUGAGCCUGACUACAGCCUGCUGCUGACCUUCAUCUACAACGGCAGAGUCGUGGGC
GAAGCCCAGGUGCAGUCUCUGGAUUGCAGACUGGUGGCCGAGCCUAGCGGAAGCGAGUCUAGUAUGGAACAGGUGCUG
UUCCCCAAGCCUGGACCUCUGGAACCCACACAGAGGCUGCUGUCUCAACUGGAAAGGGGCAUCCUGGUGGCCAGCAAU
CCUAGAGGCCUGUUCGUGCAGAGACUGUGCCCUAUUCCUAUCAGCUGGAACGCCCCUCAGGCUCCUCCUGGACCUGGA
CCACAUCUGCUGCCCAGCAAUGAGUGCGUGGAACUGUUCCGGACCGCCUACUUCUGCAGAGAUCUCGUGCGGUACUUC
CAAGGCCUGGGACCUCCUCCAAAGUUCCAAGUGACCCUGAACUUCUGGGAAGAGAGCCACGGCAGCAGCCACACACCU
CAGAAUCUGAUCACCGUGAAGAUGGAACAAGCCUUCGCCAGAUACCUGCUGGAACAGACCCCUGAACAGUGA

Hence, preferably the RNA construct comprises a sequence substantially as set out in SEQ ID No: 30, or a fragment or variant thereof.

In still another embodiment, the IRF9 dominant negative acting form of SEQ ID No:21 may be mutated by reducing it down to amino acid residues 200-308 of SEQ ID No:31 or a fragment or variant thereof (NCBI Reference Sequence: NM_006084.5; UniProtKB—Q00978 (IRF9_HUMAN)), or an orthologue thereof.

[SEQ ID No: 31]
PFQGDQRSLEFLLPPEPDYSLLLTFIYNGRVVGEAQVQSLDCRLVAEPSGSESSMEQVLFPKPGPLEPTQRLLSQLER
GILVASNPRGLFVORLCPIPISWNAPQAPPG

Therefore, preferably the RNA construct of the first aspect comprises a DNA nucleotide sequence which encodes an amino acid sequence substantially as set out in SEQ ID No: 31, or a variant or fragment thereof.

In one embodiment, this mutated IRF9 dominant negative acting form (IRF9(200-308)) is encoded by the DNA nucleotide sequence of SEQ ID No: 32, as follows:

[SEQ ID No: 32]
CCCTTTCAAGGGGATCAGAGGTCCCTGGAGTTTCTGCTTCCTCCAGAGCCAGACTACTCACTGCTGCTCACCTTCATC
TACAACGGGCGCGTGGTGGGCGAGGCCCAGGTGCAAAGCCTGGATTGCCGCCTTGTGGCTGAGCCCTCAGGCTCTGAG
AGCAGCATGGAGCAGGTGCTGTTCCCCAAGCCTGGCCCACTGGAGCCCACGCAGCGCCTGCTGAGCCAGCTTGAGAGG
GGCATCCTAGTGGCCAGCAACCCCCGAGGCCTCTTCGTGCAGCGCCTTTGCCCCATCCCCATCTCCTGGAATGCACCC
CAGGCTCCACCTGGG

Accordingly, preferably the mutated IRF9 dominant negative acting form polypeptide is encoded by the DNA nucleotide sequence substantially as set out in SEQ ID NO: 32, or a variant or fragment thereof.

Thus, the RNA construct may comprise an RNA nucleotide sequence of SEQ ID No: 33, as follows:

[SEQ ID No: 33]
CCCUUUCAAGGGGAUCAGAGGUCCCUGGAGUUUCUGCUUCCUCCAGAGCCAGACUACUCACUGCUGCUCACCUUCAUC
UACAACGGGCGCGUGGUGGGCGAGGCCCAGGUGCAAAGCCUGGAUUGCCGCCUUGUGGCUGAGCCCUCAGGCUCUGAG
AGCAGCAUGGAGCAGGUGCUGUUCCCCAAGCCUGGCCCACUGGAGCCCACGCAGCGCCUGCUGAGCCAGCUUGAGAGG
GGCAUCCUAGUGGCCAGCAACCCCCGAGGCCUCUUCGUGCAGCGCCUUUGCCCCAUCCCCAUCUCCUGGAAUGCACCC
CAGGCUCCACCUGGG

Therefore, preferably the RNA construct comprises an RNA nucleotide sequence substantially as set out in SEQ ID No: 33, or a variant or fragment thereof.

The inventors then subjected the protein sequence of SEQ ID No: 31 to codon optimisation for human expression, and one embodiment of the codon optimised nucleic acid (DNA) sequence that includes a start (ATG) and a stop (TGA) codon is provided herein as SEQ ID No: 34, as follows:

[SEQ ID No: 34]
ATGCCATTCCAGGGCGACCAGCGGAGTCTGGAATTTCTGCTGCCTCCTGAGCCTGACTACAGCCTGCTGCTGACCTTC
ATCTACAACGGCAGAGTCGTGGGCGAAGCCCAGGTGCAGTCTCTGGATTGCAGACTGGTGGCCGAGCCTAGCGGAAGC
GAGTCTAGTATGGAACAGGTGCTGTTCCCCAAGCCTGGACCTCTGGAACCCACACAGAGGCTGCTGTCTCAACTGGAA
AGGGGCATCCTGGTGGCCAGCAATCCTAGAGGCCTGTTCGTGCAGAGACTGTGCCCTATTCCTATCAGCTGGAACGCC
CCTCAGGCTCCTCCTGGATGA

Hence, preferably the RNA construct is encoded by a DNA sequence substantially as set out in SEQ ID No: 34, or a fragment or variant thereof.

In an embodiment, the RNA sequence corresponding to the codon optimised DNA sequence of SEQ ID No: 34 that includes a start (AUG) and a stop (UGA) codon is provided herein as SEQ ID No: 35, as follows:

[SEQ ID No: 35]
AUGCCAUUCCAGGGCGACCAGCGGAGUCUGGAAUUUCUGCUGCCUCCUGAGCCUGACUACAGCCUGCUGCUGACCUUC
AUCUACAACGGCAGAGUCGUGGGCGAAGCCCAGGUGCAGUCUCUGGAUUGCAGACUGGUGGCCGAGCCUAGCGGAAGC
GAGUCUAGUAUGGAACAGGUGCUGUUCCCCAAGCCUGGACCUCUGGAACCCACACAGAGGCUGCUGUCUCAACUGGAA
AGGGGCAUCCUGGUGGCCAGCAAUCCUAGAGGCCUGUUCGUGCAGAGACUGUGCCCUAUUCCUAUCAGCUGGAACGCC
CCUCAGGCUCCUCCUGGAUGA

Hence, preferably the RNA construct comprises a sequence substantially as set out in SEQ ID No: 35, or a fragment or variant thereof.

Accordingly, the at least one IMP may be the DBD of an IRF selected from a group consisting of: IRF1; IRF4; IRF5; IRF8; and IRF9. The following are examples of DNA binding domain (DBD) that would prevent binding of the whole IRF and prevent signalling, and thereby modulate the innate sensing system. In addition, the at least one IMP may be a splice variant of an IRF.

In one embodiment, the at least one IMP may be the DBD of IRF1, i.e. the DBD—Dominant negative form of IRF1 based on the DNA binding domain (DBD), IRF1(1-164) (NCBI Reference Sequence: NM_002198.3; UniProtKB—P10914 (IRF1_HUMAN)), or an orthologue thereof. (Bouker K B, et al. Interferon regulatory factor-1 (IRF-1) exhibits tumor suppressor activities in breast cancer associated with caspase activation and induction of apoptosis. Carcinogenesis. 2005 September; 26(9):1527-35. doi: 10.1093/carcin/bg1113; and Panda D, Gjinaj E, Bachu M, Squire E, Novatt H, Ozato K, Rabin R L. IRF1 Maintains Optimal Constitutive Expression of Antiviral Genes and Regulates the Early Antiviral Response. Front Immunol. 2019 May 15; 10:1019. doi: 10.3389/fimmu.2019.01019). One embodiment of the DBD protein sequence of IRF1 is represented herein as SEQ ID No: 36, as follows:

[SEQ ID No: 36]
MPITRMRMRPWLEMQINSNQIPGLIWINKEEMIFQIPWKHAAKHGWDINKDACLFRSWAIHTGRYKAGEKEPDPKTWK
ANFRCAMNSLPDIEEVKDQSRNKGSSAVRVYRMLPPLTKNORKERKSKSSRDAKSKAKRKSCGDSSPDTFSDGLSSST
LPDDHSSY

Therefore, preferably the RNA construct of the first aspect comprises a nucleotide sequence which encodes an amino acid sequence substantially as set out in SEQ ID No: 36, or a variant or fragment thereof.

In one embodiment, the DBD—Dominant negative acting form of IRF1 polypeptide is encoded by the DNA nucleotide sequence of SEQ ID No: 37, as follows:

[SEQ ID No: 37]
ATGCCCATCACTCGGATGCGCATGAGACCCTGGCTAGAGATGCAGATTAATTCCAACCAAATCCCGGGGCTCATCTGG
ATTAATAAAGAGGAGATGATCTTCCAGATCCCATGGAAGCATGCTGCCAAGCATGGCTGGGACATCAACAAGGATGCC
TGTTTGTTCCGGAGCTGGGCCATTCACACAGGCCGATACAAAGCAGGGGAAAAGGAGCCAGATCCCAAGACGTGGAAG
GCCAACTTTCGCTGTGCCATGAACTCCCTGCCAGATATCGAGGAGGTGAAAGACCAGAGCAGGAACAAGGGCAGCTCA
GCTGTGCGAGTGTACCGGATGCTTCCACCTCTCACCAAGAACCAGAGAAAAGAAAGAAAGTCGAAGTCCAGCCGAGAT
GCTAAGAGCAAGGCCAAGAGGAAGTCATGTGGGGATTCCAGCCCTGATACCTTCTCTGATGGACTCAGCAGCTCCACT
CTGCCTGATGACCACAGCAGCTAC

Accordingly, preferably the DBD—Dominant negative acting form of IRF1 polypeptide is encoded by the DNA nucleotide sequence substantially as set out in SEQ ID NO: 37, or a variant or fragment thereof.

Thus, the RNA construct may comprise an RNA nucleotide sequence of SEQ ID No: 38, as follows:

[SEQ ID No: 38]
AUGCCCAUCACUCGGAUGCGCAUGAGACCCUGGCUAGAGAUGCAGAUUAAUUCCAACCAAAUCCCGGGGCUCAUCUGG
AUUAAUAAAGAGGAGAUGAUCUUCCAGAUCCCAUGGAAGCAUGCUGCCAAGCAUGGCUGGGACAUCAACAAGGAUGCC
UGUUUGUUCCGGAGCUGGGCCAUUCACACAGGCCGAUACAAAGCAGGGGAAAAGGAGCCAGAUCCCAAGACGUGGAAG
GCCAACUUUCGCUGUGCCAUGAACUCCCUGCCAGAUAUCGAGGAGGUGAAAGACCAGAGCAGGAACAAGGGCAGCUCA
GCUGUGCGAGUGUACCGGAUGCUUCCACCUCUCACCAAGAACCAGAGAAAAGAAAGAAAGUCGAAGUCCAGCCGAGAU
GCUAAGAGCAAGGCCAAGAGGAAGUCAUGUGGGGAUUCCAGCCCUGAUACCUUCUCUGAUGGACUCAGCAGCUCCACU
CUGCCUGAUGACCACAGCAGCUAC

Therefore, preferably the RNA construct comprises an RNA nucleotide sequence substantially as set out in SEQ ID No: 38, or a variant or fragment thereof.

The inventors then subjected the protein sequence of SEQ ID No: 36 to codon optimisation for human expression, and one embodiment of the codon optimised nucleic acid (DNA) sequence that includes a start (ATG) and a stop (TGA) codon is provided herein as SEQ ID No: 39, as follows:

[SEQ ID No: 39]
ATGCCCATCACCAGAATGAGAATGCGGCCCTGGCTGGAAATGCAGATCAACAGCAATCAGATCCCCGGCCTGATCTGG
ATCAACAAAGAAGAGATGATCTTTCAGATCCCGTGGAAGCACGCCGCCAAGCACGGATGGGACATCAACAAGGACGCC
TGCCTGTTCAGAAGCTGGGCCATCCACACCGGCAGATACAAGGCCGGCGAGAAAGAGCCCGATCCTAAGACCTGGAAG
GCCAACTTCAGATGCGCCATGAACAGCCTGCCTGACATCGAGGAAGTGAAGGACCAGAGCCGGAACAAGGGATCTTCT
GCCGTGCGGGTGTACCGGATGTTGCCTCCTCTGACCAAGAACCAGCGCAAAGAGCGGAAGTCCAAGAGCAGCAGAGAT
GCCAAGAGCAAGGCCAAGAGAAAGTCCTGCGGCGACAGCAGCCCTGACACCTTTTCTGATGGCCTGAGCAGCAGCACC
CTGCCAGATGATCACAGCAGCTACTGA

Hence, preferably the RNA construct is encoded by a DNA sequence substantially as set out in SEQ ID No: 39, or a fragment or variant thereof.

In an embodiment, the RNA sequence corresponding to the codon optimised DNA sequence of SEQ ID No: 39 that includes a start (AUG) and a stop (UGA) codon is provided herein as SEQ ID No: 40, as follows:

[SEQ ID No: 40]
AUGCCCAUCACCAGAAUGAGAAUGCGGCCCUGGCUGGAAAUGCAGAUCAACAGCAAUCAGAUCCCCGGCCUGAUCUGG
AUCAACAAAGAAGAGAUGAUCUUUCAGAUCCCGUGGAAGCACGCCGCCAAGCACGGAUGGGACAUCAACAAGGACGCC
UGCCUGUUCAGAAGCUGGGCCAUCCACACCGGCAGAUACAAGGCCGGCGAGAAAGAGCCCGAUCCUAAGACCUGGAAG
GCCAACUUCAGAUGCGCCAUGAACAGCCUGCCUGACAUCGAGGAAGUGAAGGACCAGAGCCGGAACAAGGGAUCUUCU
GCCGUGCGGGUGUACCGGAUGUUGCCUCCUCUGACCAAGAACCAGCGCAAAGAGCGGAAGUCCAAGAGCAGCAGAGAU
GCCAAGAGCAAGGCCAAGAGAAAGUCCUGCGGCGACAGCAGCCCUGACACCUUUUCUGAUGGCCUGAGCAGCAGCACC
CUGCCAGAUGAUCACAGCAGCUACUGA

Hence, preferably the RNA construct comprises a sequence substantially as set out in SEQ ID No: 40, or a fragment or variant thereof.

In one embodiment, the at least one IMP may be the DBD of IRF2, i.e. the DBD—Dominant negative acting form of IRF2 based on the DNA binding domain (DBD) IRF2 (1-113) (NCBI Reference Sequence: NM_002199.3; UniProtKB—P14316 (IRF2_HUMAN), or an orthologue thereof.

IRF2 Specifically binds to the upstream regulatory region of type I IFN and IFN-inducible MHC class I genes (the interferon consensus sequence (ICS)) and represses those genes. It also acts as an activator for several genes including H4 and IL7 and constitutively binds to the ISRE promoter to activate IL7 (Oshima S., et al., Mol. Cell. Biol. 24:6298-6310(2004). One embodiment of the DBD protein sequence of IRF2 is represented herein as SEQ ID No: 232, as follows:

[SEQ ID No: 232]
MPVERMRMRPWLEEQINSNTIPGLKWLNKEKKIFQIPWMHAARHGWDVEKDAPLFRNWAIHTGKHQPGVDKPDPKTWK
ANFRCAMNSLPDIEEVKDKSIKKGNNAFRVYRMLP

Therefore, preferably the RNA construct of the first aspect comprises a nucleotide sequence which encodes an amino acid sequence substantially as set out in SEQ ID No: 232, or a variant or fragment thereof.

In one embodiment, the DBD—Dominant negative acting form of IRF2 polypeptide is encoded by the DNA nucleotide sequence of SEQ ID No: 233, as follows:

[SEQ ID No: 233]
ATGCCGGTGGAAAGGATGCGCATGCGCCCGTGGCTGGAGGAGCAGATAAACTCCAACACGATCCCGGGGCTCAAGTGG
CTTAACAAGGAAAAGAAGATTTTTCAGATCCCCTGGATGCATGCGGCTAGACATGGGTGGGATGTGGAAAAAGATGCA
CCACTCTTTAGAAACTGGGCAATCCATACAGGAAAGCATCAACCAGGAGTAGATAAACCTGATCCCAAAACATGGAAG
GCGAATTTCAGATGCGCCATGAATTCCTTGCCTGATATTGAAGAAGTCAAGGATAAAAGCATAAAGAAAGGAAATAAT
GCCTTCAGGGTCTACCGAATGCTGCCC

Accordingly, preferably the DBD—Dominant negative acting form of IRF2 polypeptide is encoded by the DNA nucleotide sequence substantially as set out in SEQ ID NO: 233, or a variant or fragment thereof.

Thus, the RNA construct may comprise an RNA nucleotide sequence of SEQ ID No: 234, as follows:

[SEQ ID No: 234]
AUGCCGGUGGAAAGGAUGCGCAUGCGCCCGUGGCUGGAGGAGCAGAUAAACUCCAACACGAUCCCGGGGCUCAAGUGG
CUUAACAAGGAAAAGAAGAUUUUUCAGAUCCCCUGGAUGCAUGCGGCUAGACAUGGGUGGGAUGUGGAAAAAGAUGCA
CCACUCUUUAGAAACUGGGCAAUCCAUACAGGAAAGCAUCAACCAGGAGUAGAUAAACCUGAUCCCAAAACAUGGAAG
GCGAAUUUCAGAUGCGCCAUGAAUUCCUUGCCUGAUAUUGAAGAAGUCAAGGAUAAAAGCAUAAAGAAAGGAAAUAAU
GCCUUCAGGGUCUACCGAAUGCUGCCC

Therefore, preferably the RNA construct comprises an RNA nucleotide sequence substantially as set out in SEQ ID No: 234, or a variant or fragment thereof.

The inventors then subjected the protein sequence of SEQ ID No: 232 to codon optimisation for human expression, and one embodiment of the codon optimised nucleic acid (DNA) sequence that includes a start (ATG) and a stop (TGA) codon is provided herein as SEQ ID No: 235, as follows:

[SEQ ID No: 235]
ATGCCCGTGGAACGGATGAGAATGAGGCCCTGGCTGGAAGAACAGATCAACAGCAACACAATCCCCGGCCTGAAGTGG
CTGAACAAAGAGAAGAAGATCTTTCAGATCCCCTGGATGCACGCCGCCAGACACGGATGGGATGTCGAGAAAGATGCC
CCTCTGTTCAGAAACTGGGCCATCCACACCGGCAAACACCAGCCTGGCGTGGACAAGCCTGATCCTAAGACCTGGAAG
GCCAACTTCAGATGCGCCATGAACAGCCTGCCTGACATCGAGGAAGTGAAGGACAAGAGCATCAAGAAGGGCAACAAC
GCCTTCCGGGTGTACAGAATGCTGCCCTGA

Hence, preferably the RNA construct is encoded by a DNA sequence substantially as set out in SEQ ID No: 235, or a fragment or variant thereof.

In an embodiment, the RNA sequence corresponding to the codon optimised DNA sequence of SEQ ID No: 235 that includes a start (AUG) and a stop (UGA) codon is provided herein as SEQ ID No: 236, as follows:

[SEQ ID No: 236]
AUGCCCGUGGAACGGAUGAGAAUGAGGCCCUGGCUGGAAGAACAGAUCAACAGCAACACAAUCCCCGGCCUGAAGUGG
CUGAACAAAGAGAAGAAGAUCUUUCAGAUCCCCUGGAUGCACGCCGCCAGACACGGAUGGGAUGUCGAGAAAGAUGCC
CCUCUGUUCAGAAACUGGGCCAUCCACACCGGCAAACACCAGCCUGGCGUGGACAAGCCUGAUCCUAAGACCUGGAAG
GCCAACUUCAGAUGCGCCAUGAACAGCCUGCCUGACAUCGAGGAAGUGAAGGACAAGAGCAUCAAGAAGGGCAACAAC
GCCUUCCGGGUGUACAGAAUGCUGCCCUGA

Hence, preferably the RNA construct comprises a sequence substantially as set out in SEQ ID No: 236, or a fragment or variant thereof.

In another embodiment, the at least one IMP may be the DBD of IRF4, i.e. the DBD which blocks IRF1 (NCBI Reference Sequence: NM_002460.4; UniProtKB—Q15306 (IRF4_HUMAN)), or an orthologue thereof. It will be known that IRF1 is a key regulatory of the interferon induction cascade (Yoshida K et al, International Immunology, Vol. 17, No. 11, pp. 1463-1471, IRF4 binding domain, blocks IRF1). One embodiment of the DBD protein sequence of IRF4 (IRF4(21-129)) is represented herein as SEQ ID No: 41, as follows:

[SEQ ID No: 41]
NGKLRQWLIDQIDSGKYPGLVWENEEKSIFRIPWKHAGKQDYNREEDAALFKAWALFKGKFREGIDKPDPPTWKTRLR
CALNKSNDFEELVERSQLDISDPYKVYRIVP

Therefore, preferably the RNA construct of the first aspect comprises a nucleotide sequence which encodes an amino acid sequence substantially as set out in SEQ ID No: 41, or a variant or fragment thereof.

In one embodiment, the DBD of IRF4 polypeptide is encoded by the DNA nucleotide sequence of SEQ ID No: 42, as follows:

[SEQ ID No: 42]
AACGGGAAGCTCCGCCAGTGGCTGATCGACCAGATCGACAGCGGCAAGTACCCCGGGCTGGTGTGGGAGAACGAGGAG
AAGAGCATCTTCCGCATCCCCTGGAAGCACGCGGGCAAGCAGGACTACAACCGCGAGGAGGACGCCGCGCTCTTCAAG
GCTTGGGCACTGTTTAAAGGAAAGTTCCGAGAAGGCATCGACAAGCCGGACCCTCCCACCTGGAAGACGCGCCTGCGG
TGCGCTTTGAACAAGAGCAATGACTTTGAGGAACTGGTTGAGCGGAGCCAGCTGGACATCTCAGACCCGTACAAAGTG
TACAGGATTGTTCCT

Accordingly, preferably the DBD of IRF4 polypeptide is encoded by the DNA nucleotide sequence substantially as set out in SEQ ID NO: 42, or a variant or fragment thereof.

Thus, the RNA construct may comprise an RNA nucleotide sequence of SEQ ID No: 43, as follows:

[SEQ ID No: 43]
AACGGGAAGCUCCGCCAGUGGCUGAUCGACCAGAUCGACAGCGGCAAGUACCCCGGGCUGGUGUGGGAGAACGAGGAG
AAGAGCAUCUUCCGCAUCCCCUGGAAGCACGCGGGCAAGCAGGACUACAACCGCGAGGAGGACGCCGCGCUCUUCAAG
GCUUGGGCACUGUUUAAAGGAAAGUUCCGAGAAGGCAUCGACAAGCCGGACCCUCCCACCUGGAAGACGCGCCUGCGG
UGCGCUUUGAACAAGAGCAAUGACUUUGAGGAACUGGUUGAGCGGAGCCAGCUGGACAUCUCAGACCCGUACAAAGUG
UACAGGAUUGUUCCU

Therefore, preferably the RNA construct comprises an RNA nucleotide sequence substantially as set out in SEQ ID No: 43, or a variant or fragment thereof.

The inventors then subjected the protein sequence of SEQ ID No: 41 to codon optimisation for human expression, and one embodiment of the codon optimised nucleic acid (DNA) sequence that includes that includes a start (ATG) and a stop (TGA) codon is provided herein as SEQ ID No: 44, as follows:

[SEQ ID No: 44]
ATGAACGGCAAGCTGCGGCAGTGGCTGATCGACCAGATCGACAGCGGCAAGTATCCTGGCCTCGTGTGGGAGAACGAG
GAAAAGTCTATCTTCAGAATCCCCTGGAAGCACGCCGGCAAGCAGGACTACAACAGAGAAGAGGACGCCGCTCTGTTC
AAGGCCTGGGCTCTGTTTAAGGGCAAGTTCAGAGAGGGCATCGACAAGCCCGATCCTCCAACCTGGAAAACCAGACTG
AGATGCGCCCTGAACAAGAGCAACGACTTCGAGGAACTGGTGGAAAGAAGCCAGCTGGACATCAGCGACCCCTACAAG
GTGTACCGGATCGTGCCTTGA

Hence, preferably the RNA construct is encoded by a DNA sequence substantially as set out in SEQ ID No: 44, or a fragment or variant thereof.

In an embodiment, the RNA sequence corresponding to the codon optimised DNA sequence of SEQ ID No: 44 that includes a start (AUG) and a stop (UGA) codon is provided herein as SEQ ID No: 45, as follows:

[SEQ ID No: 45]
AUGAACGGCAAGCUGCGGCAGUGGCUGAUCGACCAGAUCGACAGCGGCAAGUAUCCUGGCCUCGUGUGGGAGAACGAG
GAAAAGUCUAUCUUCAGAAUCCCCUGGAAGCACGCCGGCAAGCAGGACUACAACAGAGAAGAGGACGCCGCUCUGUUC
AAGGCCUGGGCUCUGUUUAAGGGCAAGUUCAGAGAGGGCAUCGACAAGCCCGAUCCUCCAACCUGGAAAACCAGACUG
AGAUGCGCCCUGAACAAGAGCAACGACUUCGAGGAACUGGUGGAAAGAAGCCAGCUGGACAUCAGCGACCCCUACAAG
GUGUACCGGAUCGUGCCUUGA

Hence, preferably the RNA construct comprises a sequence substantially as set out in SEQ ID No: 45, or a fragment or variant thereof.

In another embodiment, the at least one IMP may be IRF4 (1-129), represented herein as SEQ ID No: 257, as follows:

[SEQ ID No: 257]
MNLEGGGRGGEFGMSAVSCGNGKLRQWLIDQIDSGKYPGLVWENEEKSIFRIPWKHAGKQDYNREEDAALFKAWALFK
GKFREGIDKPDPPTWKTRLRCALNKSNDFEELVERSQLDISDPYKVYRIVP

Therefore, preferably the RNA construct of the first aspect comprises a nucleotide sequence which encodes an amino acid sequence substantially as set out in SEQ ID No: 257, or a variant or fragment thereof.

In one embodiment, the DBD of IRF4 polypeptide is encoded by the DNA nucleotide sequence of SEQ ID No: 258, as follows:

[SEQ ID No: 258]
ATGAACCTGGAGGGCGGCGGCCGAGGCGGAGAGTTCGGCATGAGCGCGGTGAGCTGCGGCAACGGGAAGCTCCGCCAG
TGGCTGATCGACCAGATCGACAGCGGCAAGTACCCCGGGCTGGTGTGGGAGAACGAGGAGAAGAGCATCTTCCGCATC
CCCTGGAAGCACGCGGGCAAGCAGGACTACAACCGCGAGGAGGACGCCGCGCTCTTCAAGGCTTGGGCACTGTTTAAA
GGAAAGTTCCGAGAAGGCATCGACAAGCCGGACCCTCCCACCTGGAAGACGCGCCTGCGGTGCGCTTTGAACAAGAGC
AATGACTTTGAGGAACTGGTTGAGCGGAGCCAGCTGGACATCTCAGACCCGTACAAAGTGTACAGGATTGTTCCT

Accordingly, preferably the DBD of IRF4 polypeptide is encoded by the DNA nucleotide sequence substantially as set out in SEQ ID NO: 258, or a variant or fragment thereof.

Thus, the RNA construct may comprise an RNA nucleotide sequence of SEQ ID No: 259, as follows:

[SEQ ID No: 259]
AUGAACCUGGAGGGCGGCGGCCGAGGCGGAGAGUUCGGCAUGAGCGCGGUGAGCUGCGGCAACGGGAAGCUCCGCCAG
UGGCUGAUCGACCAGAUCGACAGCGGCAAGUACCCCGGGCUGGUGUGGGAGAACGAGGAGAAGAGCAUCUUCCGCAUC
CCCUGGAAGCACGCGGGCAAGCAGGACUACAACCGCGAGGAGGACGCCGCGCUCUUCAAGGCUUGGGCACUGUUUAAA
GGAAAGUUCCGAGAAGGCAUCGACAAGCCGGACCCUCCCACCUGGAAGACGCGCCUGCGGUGCGCUUUGAACAAGAGC
AAUGACUUUGAGGAACUGGUUGAGCGGAGCCAGCUGGACAUCUCAGACCCGUACAAAGUGUACAGGAUUGUUCCU

Therefore, preferably the RNA construct comprises an RNA nucleotide sequence substantially as set out in SEQ ID No: 259, or a variant or fragment thereof.

The inventors then subjected the protein sequence of SEQ ID No: 257 to codon optimisation for human expression, and one embodiment of the codon optimised nucleic acid (DNA) sequence that includes that includes a start (ATG) and a stop (TGA) codon is provided herein as SEQ ID No:260, as follows:

[SEQ ID No: 260]
ATGAATCTGGAAGGCGGCGGAAGAGGCGGCGAGTTTGGAATGTCTGCCGTGTCCTGTGGCAACGGCAAGCTGAGACAG
TGGCTGATCGACCAGATCGACAGCGGCAAGTATCCTGGCCTCGTGTGGGAGAACGAGGAAAAGTCTATCTTCAGAATC
CCCTGGAAGCACGCCGGCAAGCAGGACTACAACAGAGAAGAGGACGCCGCTCTGTTCAAGGCCTGGGCTCTGTTTAAG
GGCAAGTTCAGAGAGGGCATCGACAAGCCCGATCCTCCAACCTGGAAAACCAGACTGAGATGCGCCCTGAACAAGAGC
AACGACTTCGAGGAACTGGTGGAAAGAAGCCAGCTGGACATCAGCGACCCCTACAAGGTGTACCGGATCGTGCCCTGA

Hence, preferably the RNA construct is encoded by a DNA sequence substantially as set out in SEQ ID No: 260, or a fragment or variant thereof.

In an embodiment, the RNA sequence corresponding to the codon optimised DNA sequence of SEQ ID No: 260 that includes a start (AUG) and a stop (UGA) codon is provided herein as SEQ ID No:261, as follows:

[SEQ ID No: 261]
AUGAAUCUGGAAGGCGGCGGAAGAGGCGGCGAGUUUGGAAUGUCUGCCGUGUCCUGUGGCAACGGCAAGCUGAGACAG
UGGCUGAUCGACCAGAUCGACAGCGGCAAGUAUCCUGGCCUCGUGUGGGAGAACGAGGAAAAGUCUAUCUUCAGAAUC
CCCUGGAAGCACGCCGGCAAGCAGGACUACAACAGAGAAGAGGACGCCGCUCUGUUCAAGGCCUGGGCUCUGUUUAAG
GGCAAGUUCAGAGAGGGCAUCGACAAGCCCGAUCCUCCAACCUGGAAAACCAGACUGAGAUGCGCCCUGAACAAGAGC
AACGACUUCGAGGAACUGGUGGAAAGAAGCCAGCUGGACAUCAGCGACCCCUACAAGGUGUACCGGAUCGUGCCCUGA

Hence, preferably the RNA construct comprises a sequence substantially as set out in SEQ ID No: 261, or a fragment or variant thereof.

In another embodiment, the at least one IMP may be the DBD of IRF5 (Yang L, Zhao T, Shi X, Nakhaei P, Wang Y, Sun Q, Hiscott J, Lin R. Functional analysis of a dominant negative acting mutation of interferon regulatory factor 5. PLoS One. 2009; 4(5):e5500) (NCBI Reference Sequence: NM_032643.5; UniProtKB—Q13568 (IRF5_HUMAN)), or an orthologue thereof. Both IRF5 and 7 are triggered downstream of TLR7/8. One embodiment of the DBD protein sequence of IRF5 is represented herein as SEQ ID No:46, as follows:

[SEQ ID No: 46]
MNQSIPVAPTPPRRVRLKPWLVAQVNSCQYPGLQWVNGEKKLFCIPWRHATRHGPSQDGDNTIFKAWAKETGKYTEGV
DEADPAKWKANLRCALNKSRDFRLIYDGPRDMPPQPYKIYEVCSNGPAPTDSQPPEDYSFGA

Therefore, preferably the RNA construct of the first aspect comprises a nucleotide sequence which encodes an amino acid sequence substantially as set out in SEQ ID No: 46, or a variant or fragment thereof. The sixty-eighth amino acid highlighted in bold in SEQ ID No: 46 is an Alanine in this wild-type sequence, and can be mutated to a Proline to form a dominant negative acting form of the protein (see SEQ ID No: 51).

In one embodiment, the DBD of IRF5 polypeptide (IRF5(1-140)) is encoded by the DNA nucleotide sequence of SEQ ID No: 47, as follows:

[SEQ ID No: 47]
ATGAACCAGTCCATCCCAGTGGCTCCCACCCCACCCCGCCGCGTGCGGCTGAAGCCCTGGCTGGTGGCCCAGGTGAAC
AGCTGCCAGTACCCAGGGCTTCAATGGGTCAACGGGGAAAAGAAATTATTCTGCATCCCCTGGAGGCATGCCACAAGG
CATGGTCCCAGCCAGGACGGAGATAACACCATCTTCAAGGCCTGGGCCAAGGAGACAGGGAAATACACCGAAGGCGTG
GATGAAGCCGATCCGGCCAAGTGGAAGGCCAACCTGCGCTGTGCCCTTAACAAGAGCCGGGACTTCCGCCTCATCTAC
GACGGGCCCCGGGACATGCCACCTCAGCCCTACAAGATCTACGAGGTCTGCTCCAATGGCCCTGCTCCCACAGACTCC
CAGCCCCCTGAGGATTACTCTTTTGGTGCA

Accordingly, preferably the DBD of IRF5 polypeptide is encoded by the DNA nucleotide sequence substantially as set out in SEQ ID NO: 47, or a variant or fragment thereof.

Thus, the RNA construct may comprise an RNA nucleotide sequence of SEQ ID No: 48, as follows:

[SEQ ID No: 48]
AUGAACCAGUCCAUCCCAGUGGCUCCCACCCCACCCCGCCGCGUGCGGCUGAAGCCCUGGCUGGUGGCCCAGGUGAAC
AGCUGCCAGUACCCAGGGCUUCAAUGGGUCAACGGGGAAAAGAAAUUAUUCUGCAUCCCCUGGAGGCAUGCCACAAGG
CAUGGUCCCAGCCAGGACGGAGAUAACACCAUCUUCAAGGCCUGGGCCAAGGAGACAGGGAAAUACACCGAAGGCGUG
GAUGAAGCCGAUCCGGCCAAGUGGAAGGCCAACCUGCGCUGUGCCCUUAACAAGAGCCGGGACUUCCGCCUCAUCUAC
GACGGGCCCCGGGACAUGCCACCUCAGCCCUACAAGAUCUACGAGGUCUGCUCCAAUGGCCCUGCUCCCACAGACUCC
CAGCCCCCUGAGGAUUACUCUUUUGGUGCA

Therefore, preferably the RNA construct comprises an RNA nucleotide sequence substantially as set out in SEQ ID No: 48, or a variant or fragment thereof.

The inventors then subjected the protein sequence of SEQ ID No: 46 to codon optimisation for human expression, and one embodiment of the codon optimised nucleic acid (DNA) sequence that includes a start (ATG) and a stop (TGA) codon is provided herein as SEQ ID No: 49, as follows:

[SEQ ID No: 49]
ATGAACCAGAGCATCCCCGTGGCTCCCACACCTCCTAGAAGAGTGCGACTGAAGCCTTGGCTGGTGGCCCAAGTGAAC
AGCTGTCAGTATCCTGGCCTGCAGTGGGTCAACGGCGAGAAGAAGCTGTTCTGCATCCCTTGGAGACACGCCACCAGA
CACGGCCCTTCTCAGGACGGCGACAACACCATCTTTAAGGCCTGGGCCAAAGAGACAGGCAAGTACACCGAAGGCGTG
GACGAAGCCGATCCTGCCAAGTGGAAGGCCAATCTGAGATGCGCCCTGAACAAGAGCCGGGACTTCCGGCTGATCTAC
GACGGCCCTAGAGACATGCCTCCTCAGCCTTACAAGATCTACGAAGTGTGCAGCAACGGCCCTGCTCCTACCGATTCT
CAGCCTCCTGAGGACTACAGCTTCGGCGCTTGA

Hence, preferably the RNA construct is encoded by a DNA sequence substantially as set out in SEQ ID No: 49, or a fragment or variant thereof.

In an embodiment, the RNA sequence corresponding to the codon optimised DNA sequence of SEQ ID No: 49 that includes a start (AUG) and a stop (UGA) codon is provided herein as SEQ ID No: 50, as follows:

[SEQ ID No: 50]
AUGAACCAGAGCAUCCCCGUGGCUCCCACACCUCCUAGAAGAGUGCGACUGAAGCCUUGGCUGGUGGCCCAAGUGAAC
AGCUGUCAGUAUCCUGGCCUGCAGUGGGUCAACGGCGAGAAGAAGCUGUUCUGCAUCCCUUGGAGACACGCCACCAGA
CACGGCCCUUCUCAGGACGGCGACAACACCAUCUUUAAGGCCUGGGCCAAAGAGACAGGCAAGUACACCGAAGGCGUG
GACGAAGCCGAUCCUGCCAAGUGGAAGGCCAAUCUGAGAUGCGCCCUGAACAAGAGCCGGGACUUCCGGCUGAUCUAC
GACGGCCCUAGAGACAUGCCUCCUCAGCCUUACAAGAUCUACGAAGUGUGCAGCAACGGCCCUGCUCCUACCGAUUCU
CAGCCUCCUGAGGACUACAGCUUCGGCGCUUGA

Hence, preferably the RNA construct comprises a sequence substantially as set out in SEQ ID No: 50, or a fragment or variant thereof.

In a further embodiment, the whole protein works as a dominant negative acting form when the mutated transcript encodes a version in which the sixty-eighth amino acid, Alanine, is substituted by Proline (IRF5 A68P), as highlighted in SEQ ID No:51 (NCBI Reference Sequence: NM_032643.5; UniProtKB—Q13568 (IRF5_HUMAN)), or an orthologue thereof. Accordingly, preferably the RNA construct of the first aspect comprises a nucleotide sequence which encodes an amino acid sequence substantially as set out in SEQ ID No: 51, or a variant or fragment thereof, in which the sixty-eighth amino acid, Alanine, is substituted by Proline (IRF5 A68P).

[SEQ ID No: 51]
MNQSIPVAPTPPRRVRLKPWLVAQVNSCQYPGLQWVNGEKKLFCIPWRHATRHGPSQDGDNTIFKAWPKETGKYTEGV
DEADPAKWKANLRCALNKSRDFRLIYDGPRDMPPQPYKIYEVCSNGPAPTDSQPPEDYSFGA

Therefore, preferably the RNA construct of the first aspect comprises a nucleotide sequence which encodes an amino acid sequence substantially as set out in SEQ ID No: 51, or a variant or fragment thereof.

In one embodiment, the mutated polypeptide is encoded by the DNA nucleotide sequence of SEQ ID No: 52, as follows:

[SEQ ID No: 52]
ATGAACCAGTCCATCCCAGTGGCTCCCACCCCACCCCGCCGCGTGCGGCTGAAGCCCTGGCTGGTGGCCCAGGTGAAC
AGCTGCCAGTACCCAGGGCTTCAATGGGTCAACGGGGAAAAGAAATTATTCTGCATCCCCTGGAGGCATGCCACAAGG
CATGGTCCCAGCCAGGACGGAGATAACACCATCTTCAAGGCCTGGCCCAAGGAGACAGGGAAATACACCGAAGGCGTG
GATGAAGCCGATCCGGCCAAGTGGAAGGCCAACCTGCGCTGTGCCCTTAACAAGAGCCGGGACTTCCGCCTCATCTAC
GACGGGCCCCGGGACATGCCACCTCAGCCCTACAAGATCTACGAGGTCTGCTCCAATGGCCCTGCTCCCACAGACTCC
CAGCCCCCTGAGGATTACTCTTTTGGTGCA

Accordingly, preferably the mutated polypeptide is encoded by the DNA nucleotide sequence substantially as set out in SEQ ID NO: 52, or a variant or fragment thereof.

Thus, the RNA construct may comprise an RNA nucleotide sequence of SEQ ID No: 53, as follows:

[SEQ ID No: 53]
AUGAACCAGUCCAUCCCAGUGGCUCCCACCCCACCCCGCCGCGUGCGGCUGAAGCCCUGGCUGGUGGCCCAGGUGAAC
AGCUGCCAGUACCCAGGGCUUCAAUGGGUCAACGGGGAAAAGAAAUUAUUCUGCAUCCCCUGGAGGCAUGCCACAAGG
CAUGGUCCCAGCCAGGACGGAGAUAACACCAUCUUCAAGGCCUGGCCCAAGGAGACAGGGAAAUACACCGAAGGCGUG
GAUGAAGCCGAUCCGGCCAAGUGGAAGGCCAACCUGCGCUGUGCCCUUAACAAGAGCCGGGACUUCCGCCUCAUCUAC
GACGGGCCCCGGGACAUGCCACCUCAGCCCUACAAGAUCUACGAGGUCUGCUCCAAUGGCCCUGCUCCCACAGACUCC
CAGCCCCCUGAGGAUUACUCUUUUGGUGCA

Therefore, preferably the RNA construct comprises an RNA nucleotide sequence substantially as set out in SEQ ID No: 53, or a variant or fragment thereof.

The inventors then subjected the protein sequence of SEQ ID No: 51 to codon optimisation for human expression, and one embodiment of the codon optimised nucleic acid (DNA) sequence that includes a start (ATG) and a stop (TGA) codon is provided herein as SEQ ID No: 54, as follows:

[SEQ ID No: 54]
ATGAACCAGAGCATCCCCGTGGCTCCCACACCTCCTAGAAGAGTGCGACTGAAGCCTTGGCTGGTGGCCCAAGTGAAC
AGCTGTCAGTATCCTGGCCTGCAGTGGGTCAACGGCGAGAAGAAGCTGTTCTGCATCCCTTGGAGACACGCCACCAGA
CACGGCCCTTCTCAGGACGGCGACAACACCATCTTTAAGGCCTGGcCCAAAGAGACAGGCAAGTACACCGAAGGCGTG
GACGAAGCCGATCCTGCCAAGTGGAAGGCCAATCTGAGATGCGCCCTGAACAAGAGCCGGGACTTCCGGCTGATCTAC
GACGGCCCTAGAGACATGCCTCCTCAGCCTTACAAGATCTACGAAGTGTGCAGCAACGGCCCTGCTCCTACCGATTCT
CAGCCTCCTGAGGACTACAGCTTCGGCGCTTGA

Hence, preferably the RNA construct is encoded by a DNA sequence substantially as set out in SEQ ID No: 54, or a fragment or variant thereof.

In an embodiment, the RNA sequence corresponding to the codon optimised DNA sequence of SEQ ID No: 54 that includes a start (AUG) and a stop (UGA) codon is provided herein as SEQ ID No: 55 as follows:

[SEQ ID No: 55]
AUGAACCAGAGCAUCCCCGUGGCUCCCACACCUCCUAGAAGAGUGCGACUGAAGCCUUGGCUGGUGGCCC
AAGUGAACAGCUGUCAGUAUCCUGGCCUGCAGUGGGUCAACGGCGAGAAGAAGCUGUUCUGCAUCCCUUG
GAGACACGCCACCAGACACGGCCCUUCUCAGGACGGCGACAACACCAUCUUUAAGGCCUGGCCAAAGAG
ACAGGCAAGUACACCGAAGGCGUGGACGAAGCCGAUCCUGCCAAGUGGAAGGCCAAUCUGAGAUGCGCCC
UGAACAAGAGCCGGGACUUCCGGCUGAUCUACGACGGCCCUAGAGACAUGCCUCCUCAGCCUUACAAGAU
CUACGAAGUGUGCAGCAACGGCCCUGCUCCUACCGAUUCUCAGCCUCCUGAGGACUACAGCUUCGGCGCU
UGA

Hence, preferably the RNA construct comprises a sequence substantially as set out in SEQ ID No: 55, or a fragment or variant thereof.

In one embodiment, the at least one IMP may be the DBD of IRF6, i.e. the DBD—Dominant negative acting form of IRF6 based on the DNA binding domain (DBD) (1-115) (NCBI Reference Sequence: NM_006147.3; UniProtKB—O14896 (IRF6_HUMAN) or an orthologue thereof. One embodiment of the DBD protein sequence of IRF6 is represented herein as SEQ ID No: 237, as follows:

[SEQ ID No: 237]
MALHPRRVRLKPWLVAQVDSGLYPGLIWLHRDSKRFQIPWKHATRHSPQQEEENTIFKAWAVETGKYQEGVDDPDPAK
WKAQLRCALNKSREFNLMYDGTKEVPMNPVKIYQVCD

Therefore, preferably the RNA construct of the first aspect comprises a nucleotide sequence which encodes an amino acid sequence substantially as set out in SEQ ID No: 237, or a variant or fragment thereof.

In one embodiment, the DBD—Dominant negative acting form of IRF6 polypeptide is encoded by the DNA nucleotide sequence of SEQ ID No: 238, as follows:

[SEQ ID No: 238]
ATGGCCCTCCACCCCCGCAGAGTCCGGCTAAAGCCCTGGCTGGTGGCCCAGGTGGATAGTGGCCTCTACCCTGGGCTC
ATCTGGCTACACAGGGACTCTAAACGCTTCCAGATTCCCTGGAAACATGCCACCCGGCATAGCCCTCAACAAGAAGAG
GAAAATACCATTTTTAAGGCCTGGGCTGTAGAGACAGGGAAGTACCAGGAAGGGGTGGATGACCCTGACCCAGCTAAA
TGGAAGGCCCAGCTGCGCTGTGCTCTCAATAAGAGCAGAGAATTCAACCTGATGTATGATGGCACCAAGGAGGTGCCC
ATGAACCCAGTGAAGATATATCAAGTGTGTGAC

Accordingly, preferably the DBD—Dominant negative acting form of IRF6 polypeptide is encoded by the DNA nucleotide sequence substantially as set out in SEQ ID NO: 238, or a variant or fragment thereof.

Thus, the RNA construct may comprise an RNA nucleotide sequence of SEQ ID No: 239, as follows:

[SEQ ID No: 239]
AUGGCCCUCCACCCCCGCAGAGUCCGGCUAAAGCCCUGGCUGGUGGCCCAGGUGGAUAGUGGCCUCUACCCUGGGCUC
AUCUGGCUACACAGGGACUCUAAACGCUUCCAGAUUCCCUGGAAACAUGCCACCCGGCAUAGCCCUCAACAAGAAGAG
GAAAAUACCAUUUUUAAGGCCUGGGCUGUAGAGACAGGGAAGUACCAGGAAGGGGUGGAUGACCCUGACCCAGCUAAA
UGGAAGGCCCAGCUGCGCUGUGCUCUCAAUAAGAGCAGAGAAUUCAACCUGAUGUAUGAUGGCACCAAGGAGGUGCCC
AUGAACCCAGUGAAGAUAUAUCAAGUGUGUGAC

Therefore, preferably the RNA construct comprises an RNA nucleotide sequence substantially as set out in SEQ ID No: 239, or a variant or fragment thereof.

The inventors then subjected the protein sequence of SEQ ID No: 237 to codon optimisation for human expression, and one embodiment of the codon optimised nucleic acid (DNA) sequence that includes a start (ATG) and a stop (TGA) codon is provided herein as SEQ ID No: 240, as follows:

[SEQ ID No: 240]
ATGGCCCTGCATCCTAGAAGAGTGCGGCTGAAGCCTTGGCTGGTGGCTCAAGTGGATAGCGGCCTGTATCCTGGCCTG
ATCTGGCTGCACAGAGACAGCAAGCGGTTTCAGATCCCCTGGAAGCACGCCACCAGACACAGCCCTCAGCAAGAGGAA
GAGAACACCATCTTCAAGGCCTGGGCCGTCGAGACAGGCAAGTACCAAGAAGGCGTGGACGACCCCGATCCTGCCAAA
TGGAAAGCCCAGCTGAGATGCGCCCTGAACAAGAGCCGCGAGTTCAACCTGATGTACGACGGCACCAAAGAGGTGCCC
ATGAATCCCGTGAAGATCTACCAAGTGTGCGACTGA

Hence, preferably the RNA construct is encoded by a DNA sequence substantially as set out in SEQ ID No: 240, or a fragment or variant thereof.

In an embodiment, the RNA sequence corresponding to the codon optimised DNA sequence of SEQ ID No: 240 that includes a start (AUG) and a stop (UGA) codon is provided herein as SEQ ID No: 241, as follows:

[SEQ ID No: 241]
AUGGCCCUGCAUCCUAGAAGAGUGCGGCUGAAGCCUUGGCUGGUGGCUCAAGUGGAUAGCGGCCUGUAUCCUGGCCUG
AUCUGGCUGCACAGAGACAGCAAGCGGUUUCAGAUCCCCUGGAAGCACGCCACCAGACACAGCCCUCAGCAAGAGGAA
GAGAACACCAUCUUCAAGGCCUGGGCCGUCGAGACAGGCAAGUACCAAGAAGGCGUGGACGACCCCGAUCCUGCCAAA
UGGAAAGCCCAGCUGAGAUGCGCCCUGAACAAGAGCCGCGAGUUCAACCUGAUGUACGACGGCACCAAAGAGGUGCCC
AUGAAUCCCGUGAAGAUCUACCAAGUGUGCGACUGA

Hence, preferably the RNA construct comprises a sequence substantially as set out in SEQ ID No: 241, or a fragment or variant thereof.

In another embodiment, the at least one IMP may be the DBD of IRF8, i.e. IRF-8 DBD (1-140)—(DNA binding motif, prevents binding of other IRFs to IRG promotors—Thornton A M, et al. A dominant negative mutant of an IFN regulatory factor family protein inhibits both type I and type II IFN-stimulated gene expression and antiproliferative activity of IFNs. J Immunol. 1996 Dec. 1; 157(11):5145-54.) (NCBI Reference Sequence: NM_002163; UniProtKB—Q02556 (IRF8_HUMAN)), or an orthologue thereof. One embodiment of the DBD protein sequence of IRF8 is represented herein as SEQ ID No:56, as follows:

[SEQ ID No: 56]
MCDRNGGRRLRQWLIEQIDSSMYPGLIWENEEKSMFRIPWKHAGKQDYNQEVDASIFKAWAVEKGKFKEG
DKAEPATWKTRLRCALNKSPDFEEVTDRSQLDISEPYKVYRIVPEEEQKCKLGVATAGCVNEVTEMECGR

Therefore, preferably the RNA construct of the first aspect comprises a nucleotide sequence which encodes an amino acid sequence substantially as set out in SEQ ID No: 56, or a variant or fragment thereof.

In one embodiment, the IRF8 DBD polypeptide is encoded by the DNA nucleotide sequence of SEQ ID No: 57, as follows:

[SEQ ID No: 57]
ATGTGTGACCGGAATGGTGGTCGGCGGCTTCGACAGTGGCTGATCGAGCAGATTGACAGTAGCATGTATCCAGGACTG
ATTTGGGAGAATGAGGAGAAGAGCATGTTCCGGATCCCTTGGAAACACGCTGGCAAGCAAGATTATAATCAGGAAGTG
GATGCCTCCATTTTTAAGGCCTGGGCAGTTTTTAAAGGGAAGTTTAAAGAAGGGGACAAAGCTGAACCAGCCACTTGG
AAGACGAGGTTACGCTGTGCTTTGAATAAGAGCCCAGATTTTGAGGAAGTGACGGACCGGTCCCAACTGGACATTTCC
GAGCCATACAAAGTTTACCGAATTGTTCCTGAGGAAGAGCAAAAATGCAAACTAGGCGTGGCAACTGCTGGCTGCGTG
AATGAAGTTACAGAGATGGAGTGCGGTCGC

Accordingly, preferably the IRF8 DBD polypeptide is encoded by the DNA nucleotide sequence substantially as set out in SEQ ID NO: 57, or a variant or fragment thereof.

Thus, the RNA construct may comprise an RNA nucleotide sequence of SEQ ID No: 58, as follows:

[SEQ ID No: 58]
AUGUGUGACCGGAAUGGUGGUCGGCGGCUUCGACAGUGGCUGAUCGAGCAGAUUGACAGUAGCAUGUAUCCAGGACUG
AUUUGGGAGAAUGAGGAGAAGAGCAUGUUCCGGAUCCCUUGGAAACACGCUGGCAAGCAAGAUUAUAAUCAGGAAGUG
GAUGCCUCCAUUUUUAAGGCCUGGGCAGUUUUUAAAGGGAAGUUUAAAGAAGGGGACAAAGCUGAACCAGCCACUUGG
AAGACGAGGUUACGCUGUGCUUUGAAUAAGAGCCCAGAUUUUGAGGAAGUGACGGACCGGUCCCAACUGGACAUUUCC
GAGCCAUACAAAGUUUACCGAAUUGUUCCUGAGGAAGAGCAAAAAUGCAAACUAGGCGUGGCAACUGCUGGCUGCGUG
AAUGAAGUUACAGAGAUGGAGUGCGGUCGC

Therefore, preferably the RNA construct comprises an RNA nucleotide sequence substantially as set out in SEQ ID No: 58, or a variant or fragment thereof.

The inventors then subjected the protein sequence of SEQ ID No: 56 to codon optimisation for human expression, and one embodiment of the codon optimised nucleic acid (DNA) sequence that includes a start (ATG) and a stop (TGA) codon is provided herein as SEQ ID No: 59, as follows:

[SEQ ID No: 59]
ATGTGCGACAGAAATGGCGGCAGACGGCTGAGACAGTGGCTGATCGAGCAGATCGACAGCAGCATGTACCCCGGCCTG
ATCTGGGAGAACGAAGAGAAGTCTATGTTCAGGATCCCCTGGAAGCACGCCGGCAAGCAGGACTACAATCAAGAGGTG
GACGCCAGCATCTTCAAGGCCTGGGCCGTGTTCAAGGGCAAGTTCAAAGAGGGCGACAAGGCCGAGCCTGCCACCTGG
AAAACCAGACTGAGATGCGCCCTGAACAAGAGCCCCGACTTCGAGGAAGTGACCGACAGAAGCCAGCTGGACATCAGC
GAGCCCTACAAGGTGTACCGGATCGTGCCCGAAGAGGAACAGAAATGCAAGCTGGGCGTTGCCACCGCCGGCTGTGTG
AATGAAGTGACAGAGATGGAATGCGGCCGGTGA

Hence, preferably the RNA construct is encoded by a DNA sequence substantially as set out in SEQ ID No: 59, or a fragment or variant thereof.

In an embodiment, the RNA sequence corresponding to the codon optimised DNA sequence of SEQ ID No: 59 that includes a start (AUG) and a stop (UGA) codon is provided herein as SEQ ID No: 60, as follows:

[SEQ ID No: 60]
AUGUGCGACAGAAAUGGCGGCAGACGGCUGAGACAGUGGCUGAUCGAGCAGAUCGACAGCAGCAUGUACCCCGGCCUG
AUCUGGGAGAACGAAGAGAAGUCUAUGUUCAGGAUCCCCUGGAAGCACGCCGGCAAGCAGGACUACAAUCAAGAGGUG
GACGCCAGCAUCUUCAAGGCCUGGGCCGUGUUCAAGGGCAAGUUCAAAGAGGGCGACAAGGCCGAGCCUGCCACCUGG
AAAACCAGACUGAGAUGCGCCCUGAACAAGAGCCCCGACUUCGAGGAAGUGACCGACAGAAGCCAGCUGGACAUCAGC
GAGCCCUACAAGGUGUACCGGAUCGUGCCCGAAGAGGAACAGAAAUGCAAGCUGGGCGUUGCCACCGCCGGCUGUGUG
AAUGAAGUGACAGAGAUGGAAUGCGGCCGGUGA

Hence, preferably the RNA construct comprises a sequence substantially as set out in SEQ ID No: 60, or a fragment or variant thereof.

In one embodiment, the at least one IMP may be the DBD of IRF9, i.e. IRF9 DBD (1-120). One embodiment of the DBD protein sequence of IRF9 is referred to NCBI Reference Sequence: NM_006084.5; UniProtKB—Q00978 (IRF9_HUMAN), or an orthologue thereof, and is represented herein as SEQ ID No: 61, as follows:

[SEQ ID No: 61]
MASGRARCTRKLRNWVVEQVESGQFPGVCWDDTAKTMFRIPWKHAGKQDFREDQDAAFFKAWAIFKGKYK
EGDTGGPAVWKTRLRCALNKSSEFKEVPERGRMDVAEPYKVYQLLPPGIV

Therefore, preferably the RNA construct of the first aspect comprises a nucleotide sequence which encodes an amino acid sequence substantially as set out in SEQ ID No: 61, or a variant or fragment thereof.

In one embodiment, the IRF9 DBD polypeptide is encoded by the DNA nucleotide sequence of SEQ ID No: 62, as follows:

[SEQ ID No: 62]
ATGGCATCAGGCAGGGCACGCTGCACCCGAAAACTCCGGAACTGGGTGGTGGAGCAAGTGGAGAGTGGGCAGTTTCCC
GGAGTGTGCTGGGATGATACAGCTAAGACCATGTTCCGGATTCCCTGGAAACATGCAGGCAAGCAGGACTTCCGGGAG
GACCAGGATGCTGCCTTCTTCAAGGCCTGGGCAATATTTAAGGGAAAGTATAAGGAGGGGGACACAGGAGGTCCAGCT
GTCTGGAAGACTCGCCTGCGCTGTGCACTCAACAAGAGTTCTGAATTTAAGGAGGTTCCTGAGAGGGGCCGCATGGAT
GTTGCTGAGCCCTACAAGGTGTATCAGTTGCTGCCACCAGGAATCGTC

Accordingly, preferably the IRF9 DBD polypeptide is encoded by the DNA nucleotide sequence substantially as set out in SEQ ID NO: 62, or a variant or fragment thereof.

Thus, the RNA construct may comprise an RNA nucleotide sequence of SEQ ID No: 63, as follows:

[SEQ ID No: 63]
AUGGCAUCAGGCAGGGCACGCUGCACCCGAAAACUCCGGAACUGGGUGGUGGAGCAAGUGGAGAGUGGGCAGUUUCCC
GGAGUGUGCUGGGAUGAUACAGCUAAGACCAUGUUCCGGAUUCCCUGGAAACAUGCAGGCAAGCAGGACUUCCGGGAG
GACCAGGAUGCUGCCUUCUUCAAGGCCUGGGCAAUAUUUAAGGGAAAGUAUAAGGAGGGGGACACAGGAGGUCCAGCU
GUCUGGAAGACUCGCCUGCGCUGUGCACUCAACAAGAGUUCUGAAUUUAAGGAGGUUCCUGAGAGGGGCCGCAUGGAU
GUUGCUGAGCCCUACAAGGUGUAUCAGUUGCUGCCACCAGGAAUCGUC

Therefore, preferably the RNA construct comprises an RNA nucleotide sequence substantially as set out in SEQ ID No: 63, or a variant or fragment thereof.

The inventors then subjected the protein sequence of SEQ ID No: 61 to codon optimisation for human expression, and one embodiment of the codon optimised nucleic acid (DNA) sequence that includes a start (ATG) and a stop (TGA) codon is provided herein as SEQ ID No: 64, as follows:

[SEQ ID No: 64]
ATGGCTTCTGGCAGAGCCAGATGCACCCGGAAGCTGAGAAACTGGGTCGTCGAACAGGTGGAAAGCGGACAGTTCCCT
GGCGTGTGCTGGGATGATACCGCCAAGACAATGTTCAGAATCCCCTGGAAGCACGCCGGCAAGCAGGACTTCAGAGAA
GATCAGGACGCCGCCTTCTTCAAGGCCTGGGCCATCTTCAAGGGCAAGTACAAAGAGGGCGACACAGGCGGACCTGCC
GTGTGGAAAACCAGACTGAGATGCGCCCTGAACAAGAGCAGCGAGTTCAAAGAGGTGCCCGAGCGGGGCAGAATGGAT
GTGGCCGAACCTTACAAGGTGTACCAGCTGCTGCCTCCTGGCATCGTGTGA

Hence, preferably the RNA construct is encoded by a DNA sequence substantially as set out in SEQ ID No: 64, or a fragment or variant thereof.

In an embodiment, the RNA sequence corresponding to the codon optimised DNA sequence of SEQ ID No: 64 that includes a start (AUG) and a stop (UGA) codon is provided herein as SEQ ID No: 65, as follows:

[SEQ ID No: 65]
AUGGCUUCUGGCAGAGCCAGAUGCACCCGGAAGCUGAGAAACUGGGUCGUCGAACAGGUGGAAAGCGGACAGUUCCCU
GGCGUGUGCUGGGAUGAUACCGCCAAGACAAUGUUCAGAAUCCCCUGGAAGCACGCCGGCAAGCAGGACUUCAGAGAA
GAUCAGGACGCCGCCUUCUUCAAGGCCUGGGCCAUCUUCAAGGGCAAGUACAAAGAGGGCGACACAGGCGGACCUGCC
GUGUGGAAAACCAGACUGAGAUGCGCCCUGAACAAGAGCAGCGAGUUCAAAGAGGUGCCCGAGCGGGGCAGAAUGGAU
GUGGCCGAACCUUACAAGGUGUACCAGCUGCUGCCUCCUGGCAUCGUGUGA

Hence, preferably the RNA construct comprises a sequence substantially as set out in SEQ ID No: 65, or a fragment or variant thereof.

Category 2: Inhibitors of Pathways Leading to Interferon Production and Resulting in Stimulation of Interferon-Stimulated Genes

In one embodiment, the IMP may be configured to inhibit a pathway leading to interferon production and resulting in stimulation of interferon-stimulated genes.

Hence, an inhibitor or dominant negative inhibitor of an innate signalling pathway may be a C-terminally truncated mutant of HSP90. The HSP90 mutant may be HSP90 (CDC37) (1-232) (NCBI Reference Sequence: NM_007065.4; UniProtKB—Q16543 (CDC37_HUMAN)), or an orthologue thereof, dominant negative inhibitor of IRF3 activation, i.e. IRF3-TBK1 signalling (Yang et al. Hsp90 Regulates Activation of Interferon Regulatory Factor 3 and TBK-1 Stabilization in Sendai Virus-infected Cells, Molecular Biology of the Cell Vol. 17, 1461-1471, March 2006). One embodiment of the HSP90 dominant negative form is represented herein as SEQ ID No: 81, as follows:

[SEQ ID No: 81]
MVDYSVWDHIEVSDDEDETHPNIDTASLFRWRHQARVERMEQFQKEKEELDRGCRECKRKVAECQRKLKELEVAEGGK
AELERLQAEAQQLRKEERSWEQKLEEMRKKEKSMPWNVDTLSKDGFSKSMVNTKPEKTEEDSEEVREQKHKTFVEKYE
KQIKHFGMLRRWDDSQKYLSDNVHLVCEETANYLVIWCIDLEVEEKCALMEQVAHQTIVMQFILELAKSLKVDPRA

Therefore, preferably the RNA construct of the first aspect comprises a nucleotide sequence which encodes an amino acid sequence substantially as set out in SEQ ID No: 81, or a variant or fragment thereof.

In one embodiment, the HSP90 inhibitor or dominant negative acting form polypeptide is encoded by the DNA nucleotide sequence of SEQ ID No: 82, as follows:

[SEQ ID No: 82]
ATGGTGGACTACAGCGTGTGGGACCACATTGAGGTGTCTGATGATGAAGACGAGACGCACCCCAACATCGACACGGCC
AGTCTCTTCCGCTGGCGGCATCAGGCCCGGGTGGAACGCATGGAGCAGTTCCAGAAGGAGAAGGAGGAACTGGACAGG
GGCTGCCGCGAGTGCAAGCGCAAGGTGGCCGAGTGCCAGAGGAAACTGAAGGAGCTGGAGGTGGCCGAGGGCGGCAAG
GCAGAGCTGGAGCGCCTGCAGGCCGAGGCACAGCAGCTGCGCAAGGAGGAGCGGAGCTGGGAGCAGAAGCTGGAGGAG
ATGCGCAAGAAGGAGAAGAGCATGCCCTGGAACGTGGACACGCTCAGCAAAGACGGCTTCAGCAAGAGCATGGTAAAT
ACCAAGCCCGAGAAGACGGAGGAGGACTCAGAGGAGGTGAGGGAGCAGAAACACAAGACCTTCGTGGAAAAATACGAG
AAACAGATCAAGCACTTTGGCATGCTTCGCCGCTGGGATGACAGCCAAAAGTACCTGTCAGACAACGTCCACCTGGTG
TGCGAGGAGACAGCCAATTACCTGGTCATTTGGTGCATTGACCTAGAGGTGGAGGAGAAATGTGCACTCATGGAGCAG
GTGGCCCACCAGACAATCGTCATGCAATTTATCCTGGAGCTGGCCAAGAGCCTAAAGGTGGACCCCCGGGCC

Accordingly, preferably the HSP90 inhibitor or dominant negative acting form polypeptide is encoded by the DNA nucleotide sequence substantially as set out in SEQ ID NO: 82, or a variant or fragment thereof.

Thus, the RNA construct may comprise an RNA nucleotide sequence of SEQ ID No: 83, as follows:

[SEQ ID No: 83]
AUGGUGGACUACAGCGUGUGGGACCACAUUGAGGUGUCUGAUGAUGAAGACGAGACGCACCCCAACAUCGACACGGCC
AGUCUCUUCCGCUGGCGGCAUCAGGCCCGGGUGGAACGCAUGGAGCAGUUCCAGAAGGAGAAGGAGGAACUGGACAGG
GGCUGCCGCGAGUGCAAGCGCAAGGUGGCCGAGUGCCAGAGGAAACUGAAGGAGCUGGAGGUGGCCGAGGGCGGCAAG
GCAGAGCUGGAGCGCCUGCAGGCCGAGGCACAGCAGCUGCGCAAGGAGGAGCGGAGCUGGGAGCAGAAGCUGGAGGAG
AUGCGCAAGAAGGAGAAGAGCAUGCCCUGGAACGUGGACACGCUCAGCAAAGACGGCUUCAGCAAGAGCAUGGUAAAU
ACCAAGCCCGAGAAGACGGAGGAGGACUCAGAGGAGGUGAGGGAGCAGAAACACAAGACCUUCGUGGAAAAAUACGAG
AAACAGAUCAAGCACUUUGGCAUGCUUCGCCGCUGGGAUGACAGCCAAAAGUACCUGUCAGACAACGUCCACCUGGUG
UGCGAGGAGACAGCCAAUUACCUGGUCAUUUGGUGCAUUGACCUAGAGGUGGAGGAGAAAUGUGCACUCAUGGAGCAG
GUGGCCCACCAGACAAUCGUCAUGCAAUUUAUCCUGGAGCUGGCCAAGAGCCUAAAGGUGGACCCCCGGGCC

Therefore, preferably the RNA construct comprises an RNA nucleotide sequence substantially as set out in SEQ ID No: 83, or a variant or fragment thereof.

The inventors then subjected the protein sequence of SEQ ID No: 81 to codon optimisation for human expression, and one embodiment of the codon optimised nucleic acid (DNA) sequence that includes a start (ATG) and a stop (TGA) codon is provided herein as SEQ ID No: 84, as follows:

[SEQ ID No: 84]
ATGGTGGACTACAGCGTGTGGGACCACATCGAGGTGTCCGACGACGAGGATGAGACACACCCCAACATCGATACCGCC
AGCCTGTTCAGATGGCGGCACCAGGCTAGAGTGGAACGGATGGAACAGTTCCAGAAAGAGAAAGAGGAACTGGACCGG
GGCTGCCGCGAGTGCAAAAGAAAAGTGGCCGAGTGCCAGCGGAAGCTGAAAGAACTGGAAGTGGCTGAAGGCGGCAAG
GCCGAGCTGGAAAGACTGCAGGCTGAAGCCCAGCAGCTGCGCAAAGAGGAAAGAAGCTGGGAGCAGAAACTGGAAGAG
ATGCGCAAGAAAGAAAAATCCATGCCGTGGAACGTGGACACCCTGAGCAAGGACGGCTTCAGCAAGAGCATGGTCAAC
ACCAAGCCTGAGAAAACCGAAGAGGACAGCGAGGAAGTGCGGGAACAGAAACACAAGACCTTCGTCGAGAAGTACGAG
AAGCAGATCAAGCACTTCGGCATGCTGCGGAGATGGGACGACAGCCAGAAGTACCTGAGCGACAACGTGCACCTCGTG
TGCGAGGAAACCGCCAACTACCTGGTCATCTGGTGCATCGATCTCGAGGTGGAAGAGAAGTGCGCCCTCATGGAACAG
GTGGCCCACCAGACAATCGTGATGCAGTTCATCCTGGAACTGGCCAAGAGCCTGAAGGTGGACCCTAGAGCTTGA

Hence, preferably the RNA construct is encoded by a DNA sequence substantially as set out in SEQ ID No: 84, or a fragment or variant thereof.

In an embodiment, the RNA sequence corresponding to the codon optimised DNA sequence of SEQ ID No: 84 that includes a start (AUG) and a stop (UGA) codon is provided herein as SEQ ID No: 85, as follows:

[SEQ ID No: 85]
AUGGUGGACUACAGCGUGUGGGACCACAUCGAGGUGUCCGACGACGAGGAUGAGACACACCCCAACAUCGAUACCGCC
AGCCUGUUCAGAUGGCGGCACCAGGCUAGAGUGGAACGGAUGGAACAGUUCCAGAAAGAGAAAGAGGAACUGGACCGG
GGCUGCCGCGAGUGCAAAAGAAAAGUGGCCGAGUGCCAGCGGAAGCUGAAAGAACUGGAAGUGGCUGAAGGCGGCAAG
GCCGAGCUGGAAAGACUGCAGGCUGAAGCCCAGCAGCUGCGCAAAGAGGAAAGAAGCUGGGAGCAGAAACUGGAAGAG
AUGCGCAAGAAAGAAAAAUCCAUGCCGUGGAACGUGGACACCCUGAGCAAGGACGGCUUCAGCAAGAGCAUGGUCAAC
ACCAAGCCUGAGAAAACCGAAGAGGACAGCGAGGAAGUGCGGGAACAGAAACACAAGACCUUCGUCGAGAAGUACGAG
AAGCAGAUCAAGCACUUCGGCAUGCUGCGGAGAUGGGACGACAGCCAGAAGUACCUGAGCGACAACGUGCACCUCGUG
UGCGAGGAAACCGCCAACUACCUGGUCAUCUGGUGCAUCGAUCUCGAGGUGGAAGAGAAGUGCGCCCUCAUGGAACAG
GUGGCCCACCAGACAAUCGUGAUGCAGUUCAUCCUGGAACUGGCCAAGAGCCUGAAGGUGGACCCUAGAGCUUGA

Hence, preferably the RNA construct comprises a sequence substantially as set out in SEQ ID No: 85, or a fragment or variant thereof.

In one embodiment, the inhibitor of an innate signalling pathway is STING-beta, which blocks activity of STING and is also key to the innate sensing cascade (GenBank: MF360993.1; UniProtKB—A0A3G1PSE3 (A0A3G1PSE3_HUMAN)), or an orthologue thereof (Wang P H, et al. A novel transcript isoform of STING that sequesters cGAMP and dominantly inhibits innate nucleic acid sensing. Nucleic Acids Res. 2018 May 4; 46(8):4054-4071. doi: 10.1093/nar/gky186.). STING is involved in the pathways downstream of dsRNA recognition leaving to IRF3 activation. One embodiment of the STING-beta is represented herein as SEQ ID No:86, as follows:

[SEQ ID No: 86]
MTWVSLLNQVGDRVSRNNFLGFPASELQARIRTYNQHYNNLLRGAVSQRL
YILLPLDCGVPDNLSMADPNIRFLDKLPQQTGDHAGIKDRVYSNSIYELL
ENGQRAGTCVLEYATPLQTLFAMSQYSQAGFSREDRLEQAKLFCRTLEDI
LADAPESQNNCRLIAYQEPADDSSFSLSQEVLRHLRQEEKEEVTVGSLKT
SAVPSTSTMSQEPELLISGMEKPLPLRTDFS

Therefore, preferably the RNA construct of the first aspect comprises a nucleotide sequence which encodes an amino acid sequence substantially as set out in SEQ ID No: 86, or a variant or fragment thereof.

In one embodiment, the STING-beta polypeptide is encoded by the DNA nucleotide sequence of SEQ ID No: 87, as follows:

[SEQ ID No: 87]
ATGACCTGGGTCTCACTCCTGAATCAGGTGGGAGATAGGGTTAGCAGGAA
TAACTTCTTGGGCTTCCCTGCCTCAGAGCTCCAGGCCCGGATTCGAACTT
ACAATCAGCATTACAACAACCTGCTACGGGGTGCAGTGAGCCAGCGGCTG
TATATTCTCCTCCCATTGGACTGTGGGGTGCCTGATAACCTGAGTATGGC
TGACCCCAACATTCGCTTCCTGGATAAACTGCCCCAGCAGACCGGTGACC
ATGCTGGCATCAAGGATCGGGTTTACAGCAACAGCATCTATGAGCTTCTG
GAGAACGGGCAGCGGGCGGGCACCTGTGTCCTGGAGTACGCCACCCCCTT
GCAGACTTTGTTTGCCATGTCACAATACAGTCAAGCTGGCTTTAGCCGGG
AGGATAGGCTTGAGCAGGCCAAACTCTTCTGCCGGACACTTGAGGACATC
CTGGCAGATGCCCCTGAGTCTCAGAACAACTGCCGCCTCATTGCCTACCA
GGAACCTGCAGATGACAGCAGCTTCTCGCTGTCCCAGGAGGTTCTCCGGC
ACCTGCGGCAGGAGGAAAAGGAAGAGGTTACTGTGGGCAGCTTGAAGACC
TCAGCGGTGCCCAGTACCTCCACGATGTCCCAAGAGCCTGAGCTCCTCAT
CAGTGGAATGGAAAAGCCCCTCCCTCTCCGCACGGATTTCTCT

Accordingly, preferably the STING-beta polypeptide is encoded by the DNA nucleotide sequence substantially as set out in SEQ ID NO: 87, or a variant or fragment thereof.

Thus, the RNA construct may comprise an RNA nucleotide sequence of SEQ ID No: 88, as follows:

[SEQ ID No: 88]
AUGACCUGGGUCUCACUCCUGAAUCAGGUGGGAGAUAGGGUUAGCAGGAA
UAACUUCUUGGGCUUCCCUGCCUCAGAGCUCCAGGCCCGGAUUCGAACUU
ACAAUCAGCAUUACAACAACCUGCUACGGGGUGCAGUGAGCCAGCGGCUG
UAUAUUCUCCUCCCAUUGGACUGUGGGGUGCCUGAUAACCUGAGUAUGGC
UGACCCCAACAUUCGCUUCCUGGAUAAACUGCCCCAGCAGACCGGUGACC
AUGCUGGCAUCAAGGAUCGGGUUUACAGCAACAGCAUCUAUGAGCUUCUG
GAGAACGGGCAGCGGGCGGGCACCUGUGUCCUGGAGUACGCCACCCCCUU
GCAGACUUUGUUUGCCAUGUCACAAUACAGUCAAGCUGGCUUUAGCCGGG
AGGAUAGGCUUGAGCAGGCCAAACUCUUCUGCCGGACACUUGAGGACAUC
CUGGCAGAUGCCCCUGAGUCUCAGAACAACUGCCGCCUCAUUGCCUACCA
GGAACCUGCAGAUGACAGCAGCUUCUCGCUGUCCCAGGAGGUUCUCCGGC
ACCUGCGGCAGGAGGAAAAGGAAGAGGUUACUGUGGGCAGCUUGAAGACC
UCAGCGGUGCCCAGUACCUCCACGAUGUCCCAAGAGCCUGAGCUCCUCAU
CAGUGGAAUGGAAAAGCCCCUCCCUCUCCGCACGGAUUUCUCU

Therefore, preferably the RNA construct comprises an RNA nucleotide sequence substantially as set out in SEQ ID No: 88, or a variant or fragment thereof.

The inventors then subjected the protein sequence of SEQ ID No: 88 to codon optimisation for human expression, and one embodiment of the codon optimised nucleic acid (DNA) sequence that includes a start (ATG) and a stop (TGA) codon is provided herein as SEQ ID No: 89, as follows:

[SEQ ID No: 89]
ATGACATGGGTGTCCCTGCTGAATCAAGTGGGCGACAGAGTGTCCCGGAA
CAACTTCCTGGGATTCCCTGCCAGCGAACTGCAGGCCAGAATCCGGACCT
ACAACCAGCACTACAACAACCTGCTGAGAGGCGCCGTGTCTCAGCGGCTG
TATATTCTGCTGCCTCTGGATTGCGGCGTGCCCGACAATCTGTCTATGGC
CGATCCTAATATCCGGTTCCTGGACAAGCTGCCCCAGCAGACAGGCGATC
ACGCCGGCATTAAGGACCGGGTGTACAGCAACAGCATCTACGAGCTGCTG
GAAAACGGCCAGCGAGCCGGAACATGCGTGCTGGAATATGCCACACCTCT
GCAGACCCTGTTCGCCATGAGCCAGTATAGCCAGGCCGGCTTCAGCAGAG
AGGACAGACTGGAACAGGCCAAGCTGTTCTGCCGGACACTGGAAGATATC
CTGGCCGACGCTCCTGAGAGCCAGAACAACTGTAGACTGATCGCCTACCA
AGAGCCTGCCGACGACAGCAGCTTTAGCCTGTCTCAAGAGGTGCTGCGGC
ACCTGAGACAAGAGGAAAAAGAGGAAGTCACCGTCGGCAGCCTGAAAACC
TCTGCCGTGCCTAGCACCAGCACCATGAGTCAAGAACCTGAGCTGCTGAT
CTCCGGCATGGAAAAGCCCCTGCCTCTGAGAACCGACTTCAGCTGA

Hence, preferably the RNA construct is encoded by a DNA sequence substantially as set out in SEQ ID No: 89, or a fragment or variant thereof.

In an embodiment, the RNA sequence corresponding to the codon optimised DNA sequence of SEQ ID No: 89 that includes a start (AUG) and a stop (UGA) codon is provided herein as SEQ ID No: 90, as follows:

[SEQ ID No: 90]
AUGACAUGGGUGUCCCUGCUGAAUCAAGUGGGCGACAGAGUGUCCCGGAA
CAACUUCCUGGGAUUCCCUGCCAGCGAACUGCAGGCCAGAAUCCGGACCU
ACAACCAGCACUACAACAACCUGCUGAGAGGCGCCGUGUCUCAGCGGCUG
UAUAUUCUGCUGCCUCUGGAUUGCGGCGUGCCCGACAAUCUGUCUAUGGC
CGAUCCUAAUAUCCGGUUCCUGGACAAGCUGCCCCAGCAGACAGGCGAUC
ACGCCGGCAUUAAGGACCGGGUGUACAGCAACAGCAUCUACGAGCUGCUG
GAAAACGGCCAGCGAGCCGGAACAUGCGUGCUGGAAUAUGCCACACCUCU
GCAGACCCUGUUCGCCAUGAGCCAGUAUAGCCAGGCCGGCUUCAGCAGAG
AGGACAGACUGGAACAGGCCAAGCUGUUCUGCCGGACACUGGAAGAUAUC
CUGGCCGACGCUCCUGAGAGCCAGAACAACUGUAGACUGAUCGCCUACCA
AGAGCCUGCCGACGACAGCAGCUUUAGCCUGUCUCAAGAGGUGCUGCGGC
ACCUGAGACAAGAGGAAAAAGAGGAAGUCACCGUCGGCAGCCUGAAAACC
UCUGCCGUGCCUAGCACCAGCACCAUGAGUCAAGAACCUGAGCUGCUGAU
CUCCGGCAUGGAAAAGCCCCUGCCUCUGAGAACCGACUUCAGCUGA

Hence, preferably the RNA construct comprises a sequence substantially as set out in SEQ ID No: 90, or a fragment or variant thereof.

In one embodiment, the inhibitor of an innate signalling pathway is A20 or TNFAIP3_HUMAN, a truncated or a dominant negative acting form, which inhibits TLR3 induction of IFN-beta transcription (NCBI Reference Sequence: NM_006290.4; UniProtKB—P21580 (TNAP3_HUMAN)), or an orthologue thereof (Saitoh T, et al. A20 is a negative regulator of IFN regulatory factor 3 signaling. J Immunol. 2005 Feb. 1; 174(3):1507-12. doi:10.4049/jimmunol.174.3.1507). One embodiment of the A20 or TNFAIP3_HUMAN is represented herein as SEQ ID No:91, as follows:

[SEQ ID No: 91]
AQNPMEPSVPQLSLMDVKCETPNCPFFMSVNTQPLCHECSERRQKNQNKL
PKLNSKPGPEGLPGMALGASRGEAYEPLAWNPEESTGGPHSAPPTAPSPF
LFSETTAMKCRSPGCPFTLNVQHNGFCERCHNARQLHASHAPDHTRHLDP
GKCQACLQDVTRTFNGICSTCFKRTTAEASSSLSTSLPPSCHQRSKSDPS
RLVRSPSPHSCHRAGNDAPAGCLSQAARTPGDRTGTSKCRKAGCVYFGTP
ENKGFCTLCFIEYRENKHFAAASGKVSPTASRFQNTIPCLGRECGTLGST
MFEGYCQKCFIEAQNQRFHEAKRTEEQLRSSQRRDVPRTTQSTSRPKCAR
ASCKNILACRSEELCMECQHPNQRMGPGAHRGEPAPEDPPKQRCRAPACD
HFGNAKC

Therefore, preferably the RNA construct of the first aspect comprises a nucleotide sequence which encodes an amino acid sequence substantially as set out in SEQ ID No: 91, or a variant or fragment thereof.

In one embodiment, the A20 (369-775) or TNFAIP3_HUMAN, a truncated or a dominant negative acting form polypeptide is encoded by the DNA nucleotide sequence of SEQ ID No: 92, as follows:

[SEQ ID No: 92]
GCCCAGAATCCCATGGAACCTTCCGTGCCCCAGCTTTCTCTCATGGATGT
AAAATGTGAAACGCCCAACTGCCCCTTCTTCATGTCTGTGAACACCCAGC
CTTTATGCCATGAGTGCTCAGAGAGGCGGCAAAAGAATCAAAACAAACTC
CCAAAGCTGAACTCCAAGCCGGGCCCTGAGGGGCTCCCTGGCATGGCGCT
CGGGGCCTCTCGGGGAGAAGCCTATGAGCCCTTGGCGTGGAACCCTGAGG
AGTCCACTGGGGGGCCTCATTCGGCCCCACCGACAGCACCCAGCCCTTTT
CTGTTCAGTGAGACCACTGCCATGAAGTGCAGGAGCCCCGGCTGCCCCTT
CACACTGAATGTGCAGCACAACGGATTTTGTGAACGTTGCCACAACGCCC
GGCAACTTCACGCCAGCCACGCCCCAGACCACACAAGGCACTTGGATCCC
GGGAAGTGCCAAGCCTGCCTCCAGGATGTTACCAGGACATTTAATGGGAT
CTGCAGTACTTGCTTCAAAAGGACTACAGCAGAGGCCTCCTCCAGCCTCA
GCACCAGCCTCCCTCCTTCCTGTCACCAGCGTTCCAAGTCAGATCCCTCG
CGGCTCGTCCGGAGCCCCTCCCCGCATTCTTGCCACAGAGCTGGAAACGA
CGCCCCTGCTGGCTGCCTGTCTCAAGCTGCACGGACTCCTGGGGACAGGA
CGGGGACGAGCAAGTGCAGAAAAGCCGGCTGCGTGTATTTTGGGACTCCA
GAAAACAAGGGCTTTTGCACACTGTGTTTCATCGAGTACAGAGAAAACAA
ACATTTTGCTGCTGCCTCAGGGAAAGTCAGTCCCACAGCGTCCAGGTTCC
AGAACACCATTCCGTGCCTGGGGAGGGAATGCGGCACCCTTGGAAGCACC
ATGTTTGAAGGATACTGCCAGAAGTGTTTCATTGAAGCTCAGAATCAGAG
ATTTCATGAGGCCAAAAGGACAGAAGAGCAACTGAGATCGAGCCAGCGCA
GAGATGTGCCTCGAACCACACAAAGCACCTCAAGGCCCAAGTGCGCCCGG
GCCTCCTGCAAGAACATCCTGGCCTGCCGCAGCGAGGAGCTCTGCATGGA
GTGTCAGCATCCCAACCAGAGGATGGGCCCTGGGGCCCACCGGGGTGAGC
CTGCCCCCGAAGACCCCCCCAAGCAGCGTTGCCGGGCCCCCGCCTGTGAT
CATTTTGGCAATGCCAAGTGC

Accordingly, preferably the A20 or TNFAIP3_HUMAN, a truncated or a dominant negative acting form polypeptide is encoded by the DNA nucleotide sequence substantially as set out in SEQ ID NO: 92, or a variant or fragment thereof.

Thus, the RNA construct may comprise an RNA nucleotide sequence of SEQ ID No: 93, as follows:

[SEQ ID No: 93]
GCCCAGAAUCCCAUGGAACCUUCCGUGCCCCAGCUUUCUCUCAUGGAUGU
AAAAUGUGAAACGCCCAACUGCCCCUUCUUCAUGUCUGUGAACACCCAGC
CUUUAUGCCAUGAGUGCUCAGAGAGGCGGCAAAAGAAUCAAAACAAACUC
CCAAAGCUGAACUCCAAGCCGGGCCCUGAGGGGCUCCCUGGCAUGGCGCU
CGGGGCCUCUCGGGGAGAAGCCUAUGAGCCCUUGGCGUGGAACCCUGAGG
AGUCCACUGGGGGGCCUCAUUCGGCCCCACCGACAGCACCCAGCCCUUUU
CUGUUCAGUGAGACCACUGCCAUGAAGUGCAGGAGCCCCGGCUGCCCCUU
CACACUGAAUGUGCAGCACAACGGAUUUUGUGAACGUUGCCACAACGCCC
GGCAACUUCACGCCAGCCACGCCCCAGACCACACAAGGCACUUGGAUCCC
GGGAAGUGCCAAGCCUGCCUCCAGGAUGUUACCAGGACAUUUAAUGGGAU
CUGCAGUACUUGCUUCAAAAGGACUACAGCAGAGGCCUCCUCCAGCCUCA
GCACCAGCCUCCCUCCUUCCUGUCACCAGCGUUCCAAGUCAGAUCCCUCG
CGGCUCGUCCGGAGCCCCUCCCCGCAUUCUUGCCACAGAGCUGGAAACGA
CGCCCCUGCUGGCUGCCUGUCUCAAGCUGCACGGACUCCUGGGGACAGGA
CGGGGACGAGCAAGUGCAGAAAAGCCGGCUGCGUGUAUUUUGGGACUCCA
GAAAACAAGGGCUUUUGCACACUGUGUUUCAUCGAGUACAGAGAAAACAA
ACAUUUUGCUGCUGCCUCAGGGAAAGUCAGUCCCACAGCGUCCAGGUUCC
AGAACACCAUUCCGUGCCUGGGGAGGGAAUGCGGCACCCUUGGAAGCACC
AUGUUUGAAGGAUACUGCCAGAAGUGUUUCAUUGAAGCUCAGAAUCAGAG
AUUUCAUGAGGCCAAAAGGACAGAAGAGCAACUGAGAUCGAGCCAGCGCA
GAGAUGUGCCUCGAACCACACAAAGCACCUCAAGGCCCAAGUGCGCCCGG
GCCUCCUGCAAGAACAUCCUGGCCUGCCGCAGCGAGGAGCUCUGCAUGGA
GUGUCAGCAUCCCAACCAGAGGAUGGGCCCUGGGGCCCACCGGGGUGAGC
CUGCCCCCGAAGACCCCCCCAAGCAGCGUUGCCGGGCCCCCGCCUGUGAU
CAUUUUGGCAAUGCCAAGUGC

Therefore, preferably the RNA construct comprises an RNA nucleotide sequence substantially as set out in SEQ ID No: 93, or a variant or fragment thereof.

The inventors then subjected the protein sequence of SEQ ID No: 91 to codon optimisation for human expression, and one embodiment of the codon optimised nucleic acid (DNA) sequence that includes a start (ATG) and a stop (TGA) codon is provided herein as SEQ ID No: 94, as follows:

[SEQ ID No: 94]
ATGGCCCAGAATCCTATGGAACCTAGCGTGCCCCAGCTGAGCCTGATGGA
CGTGAAGTGCGAAACCCCTAACTGCCCCTTCTTCATGTCCGTGAACACCC
AGCCTCTGTGCCACGAGTGTAGCGAGCGGAGACAGAAGAACCAGAACAAG
CTGCCCAAGCTGAACAGCAAGCCCGGACCTGAAGGACTGCCTGGAATGGC
TCTGGGAGCTTCTAGAGGCGAGGCCTATGAACCCCTGGCCTGGAATCCTG
AGGAAAGCACAGGCGGACCTCACAGCGCTCCTCCAACAGCACCTTCTCCA
TTTCTGTTCAGCGAGACAACCGCCATGAAGTGCAGAAGCCCTGGCTGCCC
TTTCACACTGAACGTGCAGCACAACGGCTTTTGCGAGAGATGCCACAACG
CCAGACAGCTGCACGCTTCTCACGCCCCTGATCACACCAGACACCTGGAT
CCTGGAAAGTGCCAGGCCTGCCTGCAGGATGTGACCAGAACCTTCAACGG
CATCTGCAGCACCTGTTTCAAGCGGACAACAGCCGAGGCCAGCAGCAGCC
TGTCTACATCTCTGCCTCCAAGCTGCCACCAGCGGAGCAAGAGCGATCCT
TCTAGACTTGTGCGGAGCCCCTCTCCTCACTCCTGTCACAGAGCCGGAAA
TGATGCCCCTGCCGGATGTCTGTCTCAGGCCGCTAGAACACCTGGCGATA
GAACCGGCACCAGCAAGTGTAGAAAGGCCGGCTGCGTGTACTTCGGCACC
CCTGAGAACAAGGGATTCTGCACCCTGTGCTTCATCGAGTACAGAGAGAA
CAAGCACTTCGCCGCTGCCTCCGGAAAGGTGTCACCTACCGCTAGCCGGT
TCCAGAACACAATCCCTTGCCTGGGCAGAGAGTGTGGCACACTGGGCAGC
ACAATGTTCGAGGGCTACTGCCAGAAGTGCTTTATCGAGGCCCAGAACCA
GCGGTTCCACGAGGCCAAGAGAACCGAGGAACAGCTGAGAAGCAGCCAGA
GAAGGGACGTGCCCAGAACAACCCAGAGCACCAGCAGACCTAAGTGCGCC
AGAGCCAGCTGCAAGAACATCCTGGCCTGTCGGAGCGAGGAACTGTGCAT
GGAATGCCAGCATCCTAACCAGAGAATGGGCCCTGGCGCTCACAGAGGCG
AACCTGCTCCAGAAGATCCTCCTAAGCAGCGGTGTAGAGCCCCTGCCTGT
GACCACTTTGGCAACGCCAAGTGCTGA

Hence, preferably the RNA construct is encoded by a DNA sequence substantially as set out in SEQ ID No: 94, or a fragment or variant thereof.

In an embodiment, the RNA sequence corresponding to the codon optimised DNA sequence of SEQ ID No: 94 that includes a start (AUG) and a stop (UGA) codon is provided herein as SEQ ID No: 95, as follows:

[SEQ ID No: 95]
AUGGCCCAGAAUCCUAUGGAACCUAGCGUGCCCCAGCUGAGCCUGAUGGA
CGUGAAGUGCGAAACCCCUAACUGCCCCUUCUUCAUGUCCGUGAACACCC
AGCCUCUGUGCCACGAGUGUAGCGAGCGGAGACAGAAGAACCAGAACAAG
CUGCCCAAGCUGAACAGCAAGCCCGGACCUGAAGGACUGCCUGGAAUGGC
UCUGGGAGCUUCUAGAGGCGAGGCCUAUGAACCCCUGGCCUGGAAUCCUG
AGGAAAGCACAGGCGGACCUCACAGCGCUCCUCCAACAGCACCUUCUCCA
UUUCUGUUCAGCGAGACAACCGCCAUGAAGUGCAGAAGCCCUGGCUGCCC
UUUCACACUGAACGUGCAGCACAACGGCUUUUGCGAGAGAUGCCACAACG
CCAGACAGCUGCACGCUUCUCACGCCCCUGAUCACACCAGACACCUGGAU
CCUGGAAAGUGCCAGGCCUGCCUGCAGGAUGUGACCAGAACCUUCAACGG
CAUCUGCAGCACCUGUUUCAAGCGGACAACAGCCGAGGCCAGCAGCAGCC
UGUCUACAUCUCUGCCUCCAAGCUGCCACCAGCGGAGCAAGAGCGAUCCU
UCUAGACUUGUGCGGAGCCCCUCUCCUCACUCCUGUCACAGAGCCGGAAA
UGAUGCCCCUGCCGGAUGUCUGUCUCAGGCCGCUAGAACACCUGGCGAUA
GAACCGGCACCAGCAAGUGUAGAAAGGCCGGCUGCGUGUACUUCGGCACC
CCUGAGAACAAGGGAUUCUGCACCCUGUGCUUCAUCGAGUACAGAGAGAA
CAAGCACUUCGCCGCUGCCUCCGGAAAGGUGUCACCUACCGCUAGCCGGU
UCCAGAACACAAUCCCUUGCCUGGGCAGAGAGUGUGGCACACUGGGCAGC
ACAAUGUUCGAGGGCUACUGCCAGAAGUGCUUUAUCGAGGCCCAGAACCA
GCGGUUCCACGAGGCCAAGAGAACCGAGGAACAGCUGAGAAGCAGCCAGA
GAAGGGACGUGCCCAGAACAACCCAGAGCACCAGCAGACCUAAGUGCGCC
AGAGCCAGCUGCAAGAACAUCCUGGCCUGUCGGAGCGAGGAACUGUGCAU
GGAAUGCCAGCAUCCUAACCAGAGAAUGGGCCCUGGCGCUCACAGAGGCG
AACCUGCUCCAGAAGAUCCUCCUAAGCAGCGGUGUAGAGCCCCUGCCUGU
GACCACUUUGGCAACGCCAAGUGCUGA

Hence, preferably the RNA construct comprises a sequence substantially as set out in SEQ ID No: 95, or a fragment or variant thereof.

In another embodiment, the inhibitor of an innate signalling pathway, a truncated or a dominant negative acting form thereof, is A20 smaller fragment (606-790), NCBI Reference Sequence: NM_006290.4; UniProtKB—P21580 (TNAP3_HUMAN), or an orthologue thereof, which prevents NF-kB activation. One embodiment of the A20 smaller fragment is represented herein as SEQ ID No: 96, as follows:

[SEQ ID No: 96]
KCRKAGCVYFGTPENKGFCTLCFIEYRENKHFAAASGKVSPTASRFQNTI
PCLGRECGTLGSTMFEGYCQKCFIEAQNQRFHEAKRTEEQLRSSQRRDVP
RTTQSTSRPKCARASCKNILACRSEELCMECQHPNQRMGPGAHRGEPAPE
DPPKQRCRAPACDHFGNAKCNGYCNECFQFKQMYG

Therefore, preferably the RNA construct of the first aspect comprises a nucleotide sequence which encodes an amino acid sequence substantially as set out in SEQ ID No: 96, or a variant or fragment thereof.

In one embodiment, the A20 smaller fragment polypeptide is encoded by the DNA nucleotide sequence of SEQ ID No: 97, as follows:

[SEQ ID No: 97]
AAGTGCAGAAAAGCCGGCTGCGTGTATTTTGGGACTCCAGAAAACAAGGG
CTTTTGCACACTGTGTTTCATCGAGTACAGAGAAAACAAACATTTTGCTG
CTGCCTCAGGGAAAGTCAGTCCCACAGCGTCCAGGTTCCAGAACACCATT
CCGTGCCTGGGGAGGGAATGCGGCACCCTTGGAAGCACCATGTTTGAAGG
ATACTGCCAGAAGTGTTTCATTGAAGCTCAGAATCAGAGATTTCATGAGG
CCAAAAGGACAGAAGAGCAACTGAGATCGAGCCAGCGCAGAGATGTGCCT
CGAACCACACAAAGCACCTCAAGGCCCAAGTGCGCCCGGGCCTCCTGCAA
GAACATCCTGGCCTGCCGCAGCGAGGAGCTCTGCATGGAGTGTCAGCATC
CCAACCAGAGGATGGGCCCTGGGGCCCACCGGGGTGAGCCTGCCCCCGAA
GACCCCCCCAAGCAGCGTTGCCGGGCCCCCGCCTGTGATCATTTTGGCAA
TGCCAAGTGCAACGGCTACTGCAACGAATGCTTTCAGTTCAAGCAGATGT
ATGGC

Accordingly, preferably the A20 smaller fragment polypeptide is encoded by the DNA nucleotide sequence substantially as set out in SEQ ID NO: 97, or a variant or fragment thereof.

Thus, the RNA construct may comprise an RNA nucleotide sequence of SEQ ID No: 98, as follows:

[SEQ ID No: 98]
AAGUGCAGAAAAGCCGGCUGCGUGUAUUUUGGGACUCCAGAAAACAAGGG
CUUUUGCACACUGUGUUUCAUCGAGUACAGAGAAAACAAACAUUUUGCUG
CUGCCUCAGGGAAAGUCAGUCCCACAGCGUCCAGGUUCCAGAACACCAUU
CCGUGCCUGGGGAGGGAAUGCGGCACCCUUGGAAGCACCAUGUUUGAAGG
AUACUGCCAGAAGUGUUUCAUUGAAGCUCAGAAUCAGAGAUUUCAUGAGG
CCAAAAGGACAGAAGAGCAACUGAGAUCGAGCCAGCGCAGAGAUGUGCCU
CGAACCACACAAAGCACCUCAAGGCCCAAGUGCGCCCGGGCCUCCUGCAA
GAACAUCCUGGCCUGCCGCAGCGAGGAGCUCUGCAUGGAGUGUCAGCAUC
CCAACCAGAGGAUGGGCCCUGGGGCCCACCGGGGUGAGCCUGCCCCCGAA
GACCCCCCCAAGCAGCGUUGCCGGGCCCCCGCCUGUGAUCAUUUUGGCAA
UGCCAAGUGCAACGGCUACUGCAACGAAUGCUUUCAGUUCAAGCAGAUGU
AUGGC

Therefore, preferably the RNA construct comprises an RNA nucleotide sequence substantially as set out in SEQ ID No: 98, or a variant or fragment thereof.

The inventors then subjected the protein sequence of SEQ ID No: 96 to codon optimisation for human expression, and one embodiment of the codon optimised nucleic acid (DNA) sequence that includes that includes a start (ATG) and a stop (TGA) codon is provided herein as SEQ ID No: 99, as follows:

[SEQ ID No: 99]
ATGAAGTGCAGAAAGGCCGGCTGCGTGTACTTCGGCACCCCTGAGAACAA
GGGCTTCTGCACCCTGTGCTTCATCGAGTACAGAGAGAACAAGCACTTCG
CTGCCGCCAGCGGAAAGGTGTCACCTACCGCCAGCAGATTCCAGAACACA
ATCCCCTGCCTGGGCAGAGAGTGTGGCACACTGGGCAGCACAATGTTCGA
GGGCTACTGCCAGAAGTGCTTTATCGAGGCCCAGAACCAGCGGTTCCACG
AGGCCAAGAGAACCGAGGAACAGCTGAGAAGCAGCCAGAGAAGGGACGTG
CCCAGAACAACCCAGAGCACCAGCAGACCTAAGTGCGCCAGAGCCAGCTG
CAAGAACATCCTGGCCTGCAGATCCGAGGAACTGTGCATGGAATGCCAGC
ATCCTAACCAGAGAATGGGCCCTGGCGCTCACAGAGGCGAACCTGCTCCA
GAAGATCCTCCTAAGCAGCGGTGTAGAGCCCCAGCCTGTGACCACTTTGG
CAACGCCAAGTGCAACGGCTACTGCAACGAGTGCTTCCAGTTCAAGCAGA
TGTACGGCTGA

Hence, preferably the RNA construct is encoded by a DNA sequence substantially as set out in SEQ ID No: 99, or a fragment or variant thereof.

In an embodiment, the RNA sequence corresponding to the codon optimised DNA sequence of SEQ ID No: 99 that includes a start (AUG) and a stop (UGA) codon is provided herein as SEQ ID No: 100, as follows:

[SEQ ID No: 100]
AUGAAGUGCAGAAAGGCCGGCUGCGUGUACUUCGGCACCCCUGAGAACAA
GGGCUUCUGCACCCUGUGCUUCAUCGAGUACAGAGAGAACAAGCACUUCG
CUGCCGCCAGCGGAAAGGUGUCACCUACCGCCAGCAGAUUCCAGAACACA
AUCCCCUGCCUGGGCAGAGAGUGUGGCACACUGGGCAGCACAAUGUUCGA
GGGCUACUGCCAGAAGUGCUUUAUCGAGGCCCAGAACCAGCGGUUCCACG
AGGCCAAGAGAACCGAGGAACAGCUGAGAAGCAGCCAGAGAAGGGACGUG
CCCAGAACAACCCAGAGCACCAGCAGACCUAAGUGCGCCAGAGCCAGCUG
CAAGAACAUCCUGGCCUGCAGAUCCGAGGAACUGUGCAUGGAAUGCCAGC
AUCCUAACCAGAGAAUGGGCCCUGGCGCUCACAGAGGCGAACCUGCUCCA
GAAGAUCCUCCUAAGCAGCGGUGUAGAGCCCCAGCCUGUGACCACUUUGG
CAACGCCAAGUGCAACGGCUACUGCAACGAGUGCUUCCAGUUCAAGCAGA
UGUACGGCUGA

Hence, preferably the RNA construct comprises a sequence substantially as set out in SEQ ID No: 100, or a fragment or variant thereof.

In another embodiment, the inhibitor/dominant negative effector of an innate signalling pathway, is the MFN2 complete polypeptide (MFN2 (1-757)), or a truncated version thereof, (NCBI Reference Sequence: NM_001127660.2; UniProtKB—O95140 (MFN2_HUMAN)), or an orthologue thereof (Yasukawa K, Oshiumi H, Takeda M, Ishihara N, Yanagi Y, Seya T, Kawabata S, Koshiba T. Mitofusin 2 inhibits mitochondrial antiviral signaling. Sci Signal. 2009 Aug. 18; 2(84):ra47. doi: 10.1126/scisignal.2000287. PMID: 19690333.).

One embodiment of the MFN2 polypeptide (MFN2 (369-598) is represented herein as SEQ ID No: 242, as follows:

[SEQ ID No: 242]
EAVRLIMDSLHMAAREQQVYCEEMREERQDRLKFIDKQLELLAQDYKLRI
KQITEEVERQVSTAMAEEIRRLSVLVDDYQMDFHPSPVVLKVYKNELHRH
IEEGLGRNMSDRCSTAITNSLQTMQQDMIDGLKPLLPVSVRSQIDMLVPR
QCFSLNYDLNCDKLCADFQEDIEFHFSLGWTMLVNRFLGPKNSRRALMGY
NDQVQRPIPLTPANPSMPPLPQGSLTQEE

Therefore, preferably the RNA construct of the first aspect comprises a nucleotide sequence which encodes an amino acid sequence substantially as set out in SEQ ID No: 242, or a variant or fragment thereof.

In one embodiment, the MFN2 polypeptide is encoded by the DNA nucleotide sequence of SEQ ID No: 243, as follows:

[SEQ ID No: 243]
GAGGCGGTTCGACTCATCATGGACTCCCTGCACATGGCGGCTCGGGAGCA
GCAGGTTTACTGCGAGGAAATGCGTGAAGAGCGGCAAGACCGACTGAAAT
TTATTGACAAACAGCTGGAGCTCTTGGCTCAAGACTATAAGCTGCGAATT
AAGCAGATTACGGAGGAAGTGGAGAGGCAGGTGTCGACTGCAATGGCCGA
GGAGATCAGGCGCCTCTCTGTACTGGTGGACGATTACCAGATGGACTTCC
ACCCTTCTCCAGTAGTCCTCAAGGTTTATAAGAATGAGCTGCACCGCCAC
ATAGAGGAAGGACTGGGTCGAAACATGTCTGACCGCTGCTCCACGGCCAT
CACCAACTCCCTGCAGACCATGCAGCAGGACATGATAGATGGCTTGAAAC
CCCTCCTTCCTGTGTCTGTGCGGAGTCAGATAGACATGCTGGTCCCACGC
CAGTGCTTCTCCCTCAACTATGACCTAAACTGTGACAAGCTGTGTGCTGC
TTCCAGGAAGACATTGAGTTCCATTTCTCTCTCGGATGGACCATGACTGG
TGAATAGGTTCCTGGGCCCCAAGAACAGCCGTCGGGCCTTGATGGGCTAC
AATGACCAGGTCCAGCGTCCCATCCCTCTGACGCCAGCCAACCCCAGCAT
GCCCCCACTGCCACAGGGCTCGCTCACCCAGGAGGAG

Accordingly, preferably the MFN2 polypeptide is encoded by the DNA nucleotide sequence substantially as set out in SEQ ID NO: 243, or a variant or fragment thereof.

Thus, the RNA construct may comprise an RNA nucleotide sequence of SEQ ID No: 244, as follows:

[SEQ ID No: 244]
GAGGCGGUUCGACUCAUCAUGGACUCCCUGCACAUGGCGGCUCGGGAGCA
GCAGGUUUACUGCGAGGAAAUGCGUGAAGAGCGGCAAGACCGACUGAAAU
UUAUUGACAAACAGCUGGAGCUCUUGGCUCAAGACUAUAAGCUGCGAAUU
AAGCAGAUUACGGAGGAAGUGGAGAGGCAGGUGUCGACUGCAAUGGCCGA
GGAGAUCAGGCGCCUCUCUGUACUGGUGGACGAUUACCAGAUGGACUUCC
ACCCUUCUCCAGUAGUCCUCAAGGUUUAUAAGAAUGAGCUGCACCGCCAC
AUAGAGGAAGGACUGGGUCGAAACAUGUCUGACCGCUGCUCCACGGCCAU
CACCAACUCCCUGCAGACCAUGCAGCAGGACAUGAUAGAUGGCUUGAAAC
CCCUCCUUCCUGUGUCUGUGCGGAGUCAGAUAGACAUGCUGGUCCCACGC
CAGUGCUUCUCCCUCAACUAUGACCUAAACUGUGACAAGCUGUGUGCUGA
CUUCCAGGAAGACAUUGAGUUCCAUUUCUCUCUCGGAUGGACCAUGCUGG
UGAAUAGGUUCCUGGGCCCCAAGAACAGCCGUCGGGCCUUGAUGGGCUAC
AAUGACCAGGUCCAGCGUCCCAUCCCUCUGACGCCAGCCAACCCCAGCAU
GCCCCCACUGCCACAGGGCUCGCUCACCCAGGAGGAG

Therefore, preferably the RNA construct comprises an RNA nucleotide sequence substantially as set out in SEQ ID No: 244, or a variant or fragment thereof.

The inventors then subjected the protein sequence of SEQ ID No: 242 to codon optimisation for human expression, and one embodiment of the codon optimised nucleic acid (DNA) sequence that includes that includes a start (ATG) and a stop (TGA) codon is provided herein as SEQ ID No: 245, as follows:

[SEQ ID No: 245]
ATGGAGGCCGTCAGACTGATCATGGACAGCCTGCATATGGCCGCCAGAGA
GCAGCAGGTCTACTGCGAGGAAATGCGGGAAGAGAGACAGGACCGGCTGA
AGTTCATCGACAAGCAGCTGGAACTGCTGGCCCAGGACTACAAGCTGCGG
ATCAAGCAGATCACCGAAGAGGTGGAAAGACAGGTGTCCACCGCCATGGC
CGAGGAAATCAGACGACTGAGCGTGCTGGTGGACGACTACCAGATGGACT
TTCACCCCTCTCCAGTGGTGCTGAAGGTGTACAAGAACGAGCTGCACCGG
CACATCGAGGAAGGCCTGGGCAGAAACATGAGCGACAGATGCAGCACCGC
CATCACCAATAGCCTGCAGACCATGCAGCAGGACATGATCGACGGCCTGA
AACCTCTGCTGCCTGTGTCCGTCAGATCCCAGATCGACATGCTGGTGCCC
AGACAGTGCTTCAGCCTGAACTACGACCTGAACTGCGACAAGCTGTGCGC
CGACTTCCAAGAGGACATCGAGTTCCACTTCAGCCTCGGCTGGACAATGC
TGGTCAACAGATTTCTGGGCCCCAAGAACAGCAGACGGGCCCTGATGGGC
TACAACGATCAGGTGCAGAGGCCCATTCCTCTGACACCCGCCAATCCTAG
CATGCCTCCACTGCCTCAGGGCAGCCTGACACAAGAAGAATGA

Hence, preferably the RNA construct is encoded by a DNA sequence substantially as set out in SEQ ID No: 245, or a fragment or variant thereof.

In an embodiment, the RNA sequence corresponding to the codon optimised DNA sequence of SEQ ID No: 245 that includes a start (AUG) and a stop (UGA) codon is provided herein as SEQ ID No: 246, as follows:

[SEQ ID No: 246]
AUGGAGGCCGUCAGACUGAUCAUGGACAGCCUGCAUAUGGCCGCCAGAGA
GCAGCAGGUCUACUGCGAGGAAAUGCGGGAAGAGAGACAGGACCGGCUGA
AGUUCAUCGACAAGCAGCUGGAACUGCUGGCCCAGGACUACAAGCUGCGG
AUCAAGCAGAUCACCGAAGAGGUGGAAAGACAGGUGUCCACCGCCAUGGC
CGAGGAAAUCAGACGACUGAGCGUGCUGGUGGACGACUACCAGAUGGACU
UUCACCCCUCUCCAGUGGUGCUGAAGGUGUACAAGAACGAGCUGCACCGG
CACAUCGAGGAAGGCCUGGGCAGAAACAUGAGCGACAGAUGCAGCACCGC
CAUCACCAAUAGCCUGCAGACCAUGCAGCAGGACAUGAUCGACGGCCUGA
AACCUCUGCUGCCUGUGUCCGUCAGAUCCCAGAUCGACAUGCUGGUGCCC
AGACAGUGCUUCAGCCUGAACUACGACCUGAACUGCGACAAGCUGUGCGC
CGACUUCCAAGAGGACAUCGAGUUCCACUUCAGCCUCGGCUGGACAAUGC
UGGUCAACAGAUUUCUGGGCCCCAAGAACAGCAGACGGGCCCUGAUGGGC
UACAACGAUCAGGUGCAGAGGCCCAUUCCUCUGACACCCGCCAAUCCUAG
CAUGCCUCCACUGCCUCAGGGCAGCCUGACACAAGAAGAAUGA

Hence, preferably the RNA construct comprises a sequence substantially as set out in SEQ ID No: 246, or a fragment or variant thereof.

One embodiment of the truncated MFN2 (MFN2 (369-490)) is represented herein as SEQ ID No:101, as follows:

[SEQ ID No: 101]
EAVRLIMDSLHMAAREQQVYCEEMREERQDRLKFIDKQLELLAQDYKLRI
KQITEEVERQVSTAMAEEIRRLSVLVDDYQMDFHPSPVVLKVYKNELHRH
IEEGLGRNMSDRCSTAITNSLQTMQQDMIDG

Therefore, preferably the RNA construct of the first aspect comprises a nucleotide sequence which encodes an amino acid sequence substantially as set out in SEQ ID No: 101, or a variant or fragment thereof.

In one embodiment, the truncated MFN2 polypeptide is encoded by the DNA nucleotide sequence of SEQ ID No: 102, as follows:

[SEQ ID No: 102]
GAGGCGGTTCGACTCATCATGGACTCCCTGCACATGGCGGCTCGGGAGCA
GCAGGTTTACTGCGAGGAAATGCGTGAAGAGCGGCAAGACCGACTGAAAT
TTATTGACAAACAGCTGGAGCTCTTGGCTCAAGACTATAAGCTGCGAATT
AAGCAGATTACGGAGGAAGTGGAGAGGCAGGTGTCGACTGCAATGGCCGA
GGAGATCAGGCGCCTCTCTGTACTGGTGGACGATTACCAGATGGACTTCC
ACCCTTCTCCAGTAGTCCTCAAGGTTTATAAGAATGAGCTGCACCGCCAC
ATAGAGGAAGGACTGGGTCGAAACATGTCTGACCGCTGCTCCACGGCCAT
CACCAACTCCCTGCAGACCATGCAGCAGGACATGATAGATGGC

Accordingly, preferably the truncated MFN2 polypeptide is encoded by the DNA nucleotide sequence substantially as set out in SEQ ID NO: 102, or a variant or fragment thereof.

Thus, the RNA construct may comprise an RNA nucleotide sequence of SEQ ID No: 103, as follows:

[SEQ ID No: 103]
GAGGCGGUUCGACUCAUCAUGGACUCCCUGCACAUGGCGGCUCGGGAGCA
GCAGGUUUACUGCGAGGAAAUGCGUGAAGAGCGGCAAGACCGACUGAAAU
UUAUUGACAAACAGCUGGAGCUCUUGGCUCAAGACUAUAAGCUGCGAAUU
AAGCAGAUUACGGAGGAAGUGGAGAGGCAGGUGUCGACUGCAAUGGCCGA
GGAGAUCAGGCGCCUCUCUGUACUGGUGGACGAUUACCAGAUGGACUUCC
ACCCUUCUCCAGUAGUCCUCAAGGUUUAUAAGAAUGAGCUGCACCGCCAC
CAUAGAGGAAGGAUGGGUCGAAACAUGUCUGACCGCUGCUCCACGGCCAU
CACCAACUCCCUGCAGACCAUGCAGCAGGACAUGAUAGAUGGC

Therefore, preferably the RNA construct comprises an RNA nucleotide sequence substantially as set out in SEQ ID No: 103, or a variant or fragment thereof.

The inventors then subjected the protein sequence of SEQ ID No: 101 to codon optimisation for human expression, and one embodiment of the codon optimised nucleic acid (DNA) sequence that includes that includes a start (ATG) and a stop (TGA) codon is provided herein as SEQ ID No: 104, as follows:

[SEQ ID No: 104]
ATGGAAGCCGTGCGGCTGATCATGGACAGCCTGCATATGGCCGCCAGAGA
GCAGCAGGTCTACTGCGAGGAAATGCGGGAAGAGAGACAGGACCGGCTGA
AGTTCATCGACAAGCAGCTGGAACTGCTGGCCCAGGACTACAAGCTGCGG
ATCAAGCAGATCACCGAAGAGGTGGAAAGACAGGTGTCCACCGCCATGGC
CGAGGAAATCAGACGACTGAGCGTGCTGGTGGACGACTACCAGATGGACT
TTCACCCCTCTCCAGTGGTGCTGAAGGTGTACAAGAACGAGCTGCACCGG
CACATCGAGGAAGGCCTGGGCAGAAACATGAGCGACAGATGCAGCACCGC
CATCACCAATAGCCTGCAGACCATGCAGCAGGACATGATCGACGGCTGA

Hence, preferably the RNA construct is encoded by a DNA sequence substantially as set out in SEQ ID No: 104, or a fragment or variant thereof.

In an embodiment, the RNA sequence corresponding to the codon optimised DNA sequence of SEQ ID No: 104 that includes a start (AUG) and a stop (UGA) codon is provided herein as SEQ ID No: 105, as follows:

[SEQ ID No: 105]
AUGGAAGCCGUGCGGCUGAUCAUGGACAGCCUGCAUAUGGCCGCCAGAGA
GCAGCAGGUCUACUGCGAGGAAAUGCGGGAAGAGAGACAGGACCGGCUGA
AGUUCAUCGACAAGCAGCUGGAACUGCUGGCCCAGGACUACAAGCUGCGG
AUCAAGCAGAUCACCGAAGAGGUGGAAAGACAGGUGUCCACCGCCAUGGC
CGAGGAAAUCAGACGACUGAGCGUGCUGGUGGACGACUACCAGAUGGACU
UUCACCCCUCUCCAGUGGUGCUGAAGGUGUACAAGAACGAGCUGCACCGG
CACAUCGAGGAAGGCCUGGGCAGAAACAUGAGCGACAGAUGCAGCACCGC
CAUCACCAAUAGCCUGCAGACCAUGCAGCAGGACAUGAUCGACGGCUGA

Hence, preferably the RNA construct comprises a sequence substantially as set out in SEQ ID No: 105, or a fragment or variant thereof.

In another embodiment, the MFN2 dominant negative acting form of SEQ ID No:101 (NCBI Reference Sequence: NM_001127660.2; UniProtKB—O95140 (MFN2_HUMAN)), or an orthologue thereof may be mutated by reducing it down to amino acid residues 400-480 of SEQ ID No:106 or a fragment or variant thereof.

[SEQ ID No: 106]
RLKFIDKQLELLAQDYKLRIKQITEEVERQVSTAMAEEIRRLSVLVDDYQ
MDFHPSPVVLKVYKNELHRHIEEGLGRNMSD

Therefore, preferably the RNA construct of the first aspect comprises a nucleotide sequence which encodes an amino acid sequence substantially as set out in SEQ ID No: 106, or a variant or fragment thereof.

In one embodiment, the truncated MFN2 polypeptide (MFN2(400-480)) is encoded by the DNA nucleotide sequence of SEQ ID No: 107, as follows:

[SEQ ID No: 107]
CGACTGAAATTTATTGACAAACAGCTGGAGCTCTTGGCTCAAGACTATAA
GCTGCGAATTAAGCAGATTACGGAGGAAGTGGAGAGGCAGGTGTCGACTG
CAATGGCCGAGGAGATCAGGCGCCTCTCTGTACTGGTGGACGATTACCAG
ATGGACTTCCACCCTTCTCCAGTAGTCCTCAAGGTTTATAAGAATGAGCT
GCACCGCCACATAGAGGAAGGACTGGGTCGAAACATGTCTGAC

Accordingly, preferably the truncated MFN2 polypeptide is encoded by the DNA nucleotide sequence substantially as set out in SEQ ID NO: 107, or a variant or fragment thereof.

Thus, the RNA construct may comprise an RNA nucleotide sequence of SEQ ID No: 108, as follows:

[SEQ ID No: 108]
CGACUGAAAUUUAUUGACAAACAGCUGGAGCUCUUGGCUCAAGACUAUAA
GCUGCGAAUUAAGCAGAUUACGGAGGAAGUGGAGAGGCAGGUGUCGACUG
CAAUGGCCGAGGAGAUCAGGCGCCUCUCUGUACUGGUGGACGAUUACCAG
AUGGACUUCCACCCUUCUCCAGUAGUCCUCAAGGUUUAUAAGAAUGAGCU
GCACCGCCACAUAGAGGAAGGACUGGGUCGAAACAUGUCUGAC

Therefore, preferably the RNA construct comprises an RNA nucleotide sequence substantially as set out in SEQ ID No: 108, or a variant or fragment thereof.

The inventors then subjected the protein sequence of SEQ ID No: 106 to codon optimisation for human expression, and one embodiment of the codon optimised nucleic acid (DNA) sequence that includes that includes a start (ATG) and a stop (TGA) codon is provided herein as SEQ ID No: 109, as follows:

[SEQ ID No: 109]
ATGCGGCTGAAGTTCATCGACAAGCAGCTGGAACTGCTGGCCCAGGACTA
CAAGCTGCGGATCAAGCAGATCACCGAAGAGGTGGAAAGACAGGTGTCCA
CCGCCATGGCCGAGGAAATCAGACGACTGAGCGTGCTGGTGGACGACTAC
CAGATGGACTTTCACCCCTCTCCAGTGGTGCTGAAGGTGTACAAGAACGA
GCTGCACCGGCACATCGAGGAAGGCCTGGGCAGAAACATGAGCGACTGA

Hence, preferably the RNA construct is encoded by a DNA sequence substantially as set out in SEQ ID No: 109, or a fragment or variant thereof.

In an embodiment, the RNA sequence corresponding to the codon optimised DNA sequence of SEQ ID No: 109 that includes a start (AUG) and a stop (UGA) codon is provided herein as SEQ ID No: 110, as follows:

[SEQ ID No: 110]
AUGCGGCUGAAGUUCAUCGACAAGCAGCUGGAACUGCUGGCCCAGGACUA
CAAGCUGCGGAUCAAGCAGAUCACCGAAGAGGUGGAAAGACAGGUGUCCA
CCGCCAUGGCCGAGGAAAUCAGACGACUGAGCGUGCUGGUGGACGACUAC
CAGAUGGACUUUCACCCCUCUCCAGUGGUGCUGAAGGUGUACAAGAACGA
GCUGCACCGGCACAUCGAGGAAGGCCUGGGCAGAAACAUGAGCGACUGA

Hence, preferably the RNA construct comprises a sequence substantially as set out in SEQ ID No: 10, or a fragment or variant thereof.

In one embodiment, the at least one IMP may be FAF1 polypeptide (accession number—NCBI reference sequence: NM_007051.3; UniProtKB—Q9UNN5 (FAF1_HUMAN)), or truncated version or an orthologue thereof. FAF1 inhibits translocation of interferon regulatory factor 3 to the nucleus and reduces IFNβ production (Song S, Lee J-J, Kim H-J, Lee J-Y et al. Fas-Associated Factor 1 Negatively Regulates the Antiviral Immune Response by Inhibiting Translocation of Interferon Regulatory Factor 3 to the Nucleus. 2016 Jan. 25; 36(7):1136-51. doi: 10.1128/MCB.00744-15). One embodiment of FAF1 is represented herein as SEQ ID No: 146, as follows:

[SEQ ID No: 146]
MASNMDREMILADFQACTGIENIDEAITLLEQNNWDLVAA
INGVIPQENGILQSEYGGETIPGPAFNPASHPASAPTSSS
SSAFRPVMPSRQIVERQPRMLDFRVEYRDRNVDVVLEDTC
TVGEIKQILENELQIPVSKMLLKGWKTGDVEDSTVLKSLH
LPKNNSLYVLTPDLPPPSSSSHAGALQESLNQNFMLIITH
REVQREYNLNFSGSSTIQEVKRNVYDLTSIPVRHQLWEGW
PTSATDDSMCLAESGLSYPCHRLTVGRRSSPAQTREQSEE
QITDVHMVSDSDGDDFEDATEFGVDDGEVFGMASSALRKS
PMMPENAENEGDALLQFTAEFSSRYGDCHPVFFIGSLEAA
FQEAFYVKARDRKLLAIYLHHDESVLINVFCSQMLCAESI
VSYLSQNFITWAWDLTKDSNRARFLTMCNRHFGSVVAQTI
RTQKTDQFPLFLIIMGKRSSNEVLNVIQGNTTVDELMMRL
MAAMEIFTAQQQEDIKDEDEREARENVKREQDEAYRLSLE
ADRAKREAHEREMAEQFRLEQIRKEQEEEREAIRLSLEQA
LPPEPKEENAEPVSKLRIRTPSGEFLERRFLASNKLQIVF
DFVASKGFPWDEYKLLSTFPRRDVTQLDPNKSLLEVKLFP
QETLFLEAKE

Therefore, preferably the RNA construct of the first aspect comprises a nucleotide sequence which encodes an amino acid sequence substantially as set out in SEQ ID No: 146, or a variant or fragment thereof.

In one embodiment, the FAF1 polypeptide is encoded by the DNA nucleotide sequence of SEQ ID No: 147, as follows:

[SEQ ID No: 147]
ATGGCGTCCAACATGGACCGGGAGATGATCCTGGCGGATT
TTCAGGCATGTACTGGCATTGAAAACATTGACGAAGCTAT
TACATTGCTTGAACAAAATAATTGGGACTTAGTGGCAGCT
ATCAATGGTGTAATACCACAGGAAAATGGCATTCTACAAA
GTGAATATGGAGGTGAGACCATACCAGGACCTGCATTTAA
TCCAGCAAGTCATCCAGCTTCAGCTCCTACTTCCTCTTCT
TCTTCAGCGTTTCGACCTGTAATGCCATCCAGGCAGATTG
TAGAAAGGCAACCTCGGATGCTGGACTTCAGGGTTGAATA
CAGAGACAGAAATGTTGATGTGGTACTTGAAGACACCTGT
ACTGTTGGAGAGATTAAACAGATTCTAGAAAATGAACTTC
AGATACCTGTGTCCAAAATGCTGTTAAAAGGCTGGAAGAC
GGGAGATGTGGAAGACAGTACGGTCCTAAAATCTCTACAC
TTGCCAAAAAACAACAGTCTTTATGTCCTTACACCAGATT
TGCCACCACCTTCATCATCTAGTCATGCTGGTGCCCTGCA
GGAGTCATTAAATCAAAACTTCATGCTGATCATCACCCAC
CGAGAAGTCCAGCGGGAGTACAACCTGAACTTCTCAGGAA
GCAGTACTATTCAAGAGGTAAAGAGAAATGTGTATGACCT
TACAAGTATCCCCGTTCGCCACCAATTATGGGAGGGCTGG
CCAACTTCTGCTACAGACGACTCAATGTGTCTTGCTGAAT
CAGGGCTCTCTTATCCCTGCCATCGACTTACAGTGGGAAG
AAGATCTTCACCTGCACAGACCCGGGAACAGTCGGAAGAA
CAAATCACCGATGTTCATATGGTTAGTGATAGCGATGGAG
ATGACTTTGAAGATGCTACAGAATTTGGGGTGGATGATGG
AGAAGTATTTGGCATGGCGTCATCTGCCTTGAGAAAATCT
CCAATGATGCCAGAAAACGCAGAAAATGAAGGAGATGCCT
TATTACAATTTACAGCAGAGTTTTCTTCAAGATATGGTGA
TTGCCATCCTGTATTTTTTATTGGCTCATTAGAAGCTGCT
TTTCAAGAGGCCTTCTATGTGAAAGCCCGAGATAGAAAGC
TTCTTGCTATCTACCTCCACCATGATGAAAGTGTGTTAAC
CAACGTGTTCTGCTCACAAATGCTTTGTGCTGAATCCATT
GTTTCTTATCTGAGTCAAAATTTTATAACCTGGGCTTGGG
ATCTGACAAAGGACTCCAACAGAGCAAGATTTCTCACTAT
GTGCAATAGACACTTTGGCAGTGTTGTGGCACAAACCATT
CGGACTCAAAAAACGGATCAGTTTCCGCTTTTCCTGATTA
TTATGGGAAAGCGATCATCTAATGAAGTGTTGAATGTGAT
ACAAGGGAACACAACAGTAGATGAGTTAATGATGAGACTC
ATGGCTGCAATGGAGATCTTCACAGCCCAACAACAGGAAG
ATATAAAGGACGAGGATGAACGTGAAGCCAGAGAAAATGT
GAAGAGAGAGCAAGATGAGGCCTATCGCCTTTCACTTGAG
GCTGACAGAGCAAAGAGGGAAGCTCACGAGAGAGAGATGG
CAGAACAGTTTCGTTTGGAGCAGATTCGCAAAGAACAAGA
AGAGGAACGTGAGGCCATCCGGCTGTCCTTAGAGCAAGCC
CTGCCTCCTGAGCCAAAGGAAGAAAATGCTGAGCCTGTGA
GCAAACTGCGGATCCGGACCCCCAGTGGCGAGTTCTTGGA
GCGGCGTTTCCTGGCCAGCAACAAGCTCCAGATTGTCTTT
GATTTTGTAGCTTCCAAAGGATTTCCATGGGATGAGTACA
AGTTACTGAGCACCTTTCCTAGGAGAGACGTAACTCAACT
GGACCCAAATAAATCATTATTGGAGGTAAAGTTGTTCCCT
CAAGAAACCCTTTTCCTTGAAGCAAAAGAG

Accordingly, preferably the FAF1 polypeptide is encoded by the DNA nucleotide sequence substantially as set out in SEQ ID NO: 147, or a variant or fragment thereof.

Thus, the RNA construct may comprise an RNA nucleotide sequence of SEQ ID No: 148, as follows:

[SEQ ID No: 148]
AUGGCGUCCAACAUGGACCGGGAGAUGAUCCUGGCGGAUU
UUCAGGCAUGUACUGGCAUUGAAAACAUUGACGAAGCUAU
UACAUUGCUUGAACAAAAUAAUUGGGACUUAGUGGCAGCU
AUCAAUGGUGUAAUACCACAGGAAAAUGGCAUUCUACAAA
GUGAAUAUGGAGGUGAGACCAUACCAGGACCUGCAUUUAA
UCCAGCAAGUCAUCCAGCUUCAGCUCCUACUUCCUCUUCU
UCUUCAGCGUUUCGACCUGUAAUGCCAUCCAGGCAGAUUG
UAGAAAGGCAACCUCGGAUGCUGGACUUCAGGGUUGAAUA
CAGAGACAGAAAUGUUGAUGUGGUACUUGAAGACACCUGU
ACUGUUGGAGAGAUUAAACAGAUUCUAGAAAAUGAACUUC
AGAUACCUGUGUCCAAAAUGCUGUUAAAAGGCUGGAAGAC
GGGAGAUGUGGAAGACAGUACGGUCCUAAAAUCUCUACAC
UUGCCAAAAAACAACAGUCUUUAUGUCCUUACACCAGAUU
UGCCACCACCUUCAUCAUCUAGUCAUGCUGGUGCCCUGCA
GGAGUCAUUAAAUCAAAACUUCAUGCUGAUCAUCACCCAC
CGAGAAGUCCAGCGGGAGUACAACCUGAACUUCUCAGGAA
GCAGUACUAUUCAAGAGGUAAAGAGAAAUGUGUAUGACCU
UACAAGUAUCCCCGUUCGCCACCAAUUAUGGGAGGGCUGG
CCAACUUCUGCUACAGACGACUCAAUGUGUCUUGCUGAAU
CAGGGCUCUCUUAUCCCUGCCAUCGACUUACAGUGGGAAG
AAGAUCUUCACCUGCACAGACCCGGGAACAGUCGGAAGAA
CAAAUCACCGAUGUUCAUAUGGUUAGUGAUAGCGAUGGAG
AUGACUUUGAAGAUGCUACAGAAUUUGGGGUGGAUGAUGG
AGAAGUAUUUGGCAUGGCGUCAUCUGCCUUGAGAAAAUCU
CCAAUGAUGCCAGAAAACGCAGAAAAUGAAGGAGAUGCCU
UAUUACAAUUUACAGCAGAGUUUUCUUCAAGAUAUGGUGA
UUGCCAUCCUGUAUUUUUUAUUGGCUCAUUAGAAGCUGCU
UUUCAAGAGGCCUUCUAUGUGAAAGCCCGAGAUAGAAAGC
UUCUUGCUAUCUACCUCCACCAUGAUGAAAGUGUGUUAAC
CAACGUGUUCUGCUCACAAAUGCUUUGUGCUGAAUCCAUU
GUUUCUUAUCUGAGUCAAAAUUUUAUAACCUGGGCUUGGG
AUCUGACAAAGGACUCCAACAGAGCAAGAUUUCUCACUAU
GUGCAAUAGACACUUUGGCAGUGUUGUGGCACAAACCAUU
CGGACUCAAAAAACGGAUCAGUUUCCGCUUUUCCUGAUUA
UUAUGGGAAAGCGAUCAUCUAAUGAAGUGUUGAAUGUGAU
ACAAGGGAACACAACAGUAGAUGAGUUAAUGAUGAGACUC
AUGGCUGCAAUGGAGAUCUUCACAGCCCAACAACAGGAAG
AUAUAAAGGACGAGGAUGAACGUGAAGCCAGAGAAAAUGU
GAAGAGAGAGCAAGAUGAGGCCUAUCGCCUUUCACUUGAG
GCUGACAGAGCAAAGAGGGAAGCUCACGAGAGAGAGAUGG
CAGAACAGUUUCGUUUGGAGCAGAUUCGCAAAGAACAAGA
AGAGGAACGUGAGGCCAUCCGGCUGUCCUUAGAGCAAGCC
CUGCCUCCUGAGCCAAAGGAAGAAAAUGCUGAGCCUGUGA
GCAAACUGCGGAUCCGGACCCCCAGUGGCGAGUUCUUGGA
GCGGCGUUUCCUGGCCAGCAACAAGCUCCAGAUUGUCUUU
GAUUUUGUAGCUUCCAAAGGAUUUCCAUGGGAUGAGUACA
AGUUACUGAGCACCUUUCCUAGGAGAGACGUAACUCAACU
GGACCCAAAUAAAUCAUUAUUGGAGGUAAAGUUGUUCCCU
CAAGAAACCCUUUUCCUUGAAGCAAAAGAG

Therefore, preferably the RNA construct comprises an RNA nucleotide sequence substantially as set out in SEQ ID No: 148, or a variant or fragment thereof.

The inventors then subjected the protein sequence of SEQ ID No: 146 to codon optimisation for human expression, and one embodiment of the codon optimised nucleic acid (DNA) sequence that includes a start (ATG) and a stop (TGA) codon is provided herein as SEQ ID No: 149, as follows:

[SEQ ID No: 149]
ATGGCCAGCAACATGGACAGAGAGATGATCCTGGCCGACT
TCCAGGCCTGTACCGGCATCGAGAACATCGACGAGGCCAT
CACACTGCTGGAACAGAACAACTGGGATCTCGTGGCCGCC
ATCAACGGCGTGATCCCTCAAGAGAATGGCATCCTGCAGA
GCGAGTACGGCGGCGAGACAATTCCTGGACCTGCCTTCAA
TCCCGCCAGCCATCCTGCATCTGCCCCTACATCTAGCAGC
AGCAGCGCCTTCAGACCCGTGATGCCTAGCAGACAGATCG
TGGAACGGCAGCCCAGAATGCTGGACTTCAGAGTCGAGTA
CCGGGACAGAAACGTGGACGTGGTGCTGGAAGATACCTGC
ACCGTGGGCGAGATCAAGCAGATCCTGGAAAACGAGCTGC
AGATCCCCGTGTCCAAGATGCTGCTGAAAGGCTGGAAAAC
CGGCGACGTGGAAGATAGCACCGTGCTGAAGTCCCTGCAT
CTCCCTAAGAACAACAGCCTGTACGTGCTGACCCCTGACC
TGCCTCCTCCAAGCTCTAGTTCTCATGCTGGCGCCCTGCA
AGAGAGCCTGAACCAGAACTTCATGCTGATCATCACCCAC
CGCGAGGTGCAGAGAGAGTATAACCTGAACTTCAGCGGCA
GCAGCACCATCCAAGAAGTGAAGCGGAACGTCTACGACCT
GACCAGCATTCCTGTGCGGCACCAGCTTTGGGAAGGCTGG
CCTACAAGCGCCACCGACGATTCTATGTGTCTGGCCGAGA
GCGGCCTGAGCTACCCTTGTCACAGACTGACCGTGGGCAG
AAGAAGCAGCCCTGCTCAGACAAGAGAGCAGTCCGAGGAA
CAGATCACCGACGTGCACATGGTGTCCGATAGCGACGGCG
ACGATTTCGAGGATGCCACCGAGTTTGGAGTGGACGACGG
CGAGGTTTTCGGCATGGCTAGCAGCGCCCTGAGAAAGTCC
CCTATGATGCCCGAGAACGCCGAGAATGAAGGCGACGCCC
TGCTGCAGTTTACCGCCGAGTTTAGCAGCAGATACGGCGA
CTGTCACCCCGTGTTCTTCATCGGATCTCTGGAAGCCGCC
TTCCAAGAGGCCTTTTACGTGAAGGCCAGAGACAGAAAGC
TGCTGGCTATCTATCTGCACCACGACGAGAGCGTGCTGAC
AAACGTGTTCTGCAGCCAGATGCTGTGCGCCGAGAGCATC
GTGTCTTACCTGTCTCAGAATTTCATCACCTGGGCCTGGG
ATCTGACCAAGGACAGCAACAGAGCCCGGTTCCTGACCAT
GTGTAACCGGCACTTTGGCAGCGTGGTGGCCCAGACCATC
AGAACCCAGAAAACCGATCAGTTCCCTCTGTTCCTGATCA
TTATGGGCAAGCGCAGCAGCAACGAGGTGCTGAATGTGAT
CCAGGGCAACACCACCGTGGACGAGCTGATGATGAGACTG
ATGGCCGCTATGGAAATCTTCACAGCCCAGCAGCAAGAAG
ATATCAAGGACGAGGACGAGCGCGAGGCCCGCGAGAATGT
GAAAAGAGAACAGGACGAAGCCTACCGGCTGAGCCTGGAA
GCTGACAGAGCCAAGAGAGAGGCCCACGAGAGAGAGATGG
CCGAGCAGTTCAGACTCGAGCAGATCCGCAAAGAGCAAGA
GGAAGAGAGAGAAGCCATCCGGCTGTCCCTGGAACAAGCC
TTGCCTCCTGAGCCTAAAGAAGAGAACGCTGAGCCAGTGT
CCAAGCTGCGGATCAGAACTCCTAGCGGCGAGTTCCTGGA
AAGACGGTTCCTGGCCTCCAACAAACTGCAGATCGTGTTC
GACTTCGTGGCCTCTAAGGGCTTCCCCTGGGACGAGTACA
AGCTGCTGAGCACATTCCCCAGACGGGACGTGACACAGCT
GGACCCTAACAAGAGCCTGCTGGAAGTGAAACTGTTTCCC
CAAGAGACACTGTTTCTCGAGGCCAAAGAGTGA

Hence, preferably the RNA construct is encoded by a DNA sequence substantially as set out in SEQ ID No: 149, or a fragment or variant thereof.

In an embodiment, the RNA sequence corresponding to the codon optimised DNA sequence of SEQ ID No: 149 that includes a start (AUG) and a stop (UGA) codon is provided herein as SEQ ID No: 150, as follows:

[SEQ ID No: 150]
AUGGCCAGCAACAUGGACAGAGAGAUGAUCCUGGCCGACU
UCCAGGCCUGUACCGGCAUCGAGAACAUCGACGAGGCCAU
CACACUGCUGGAACAGAACAACUGGGAUCUCGUGGCCGCC
AUCAACGGCGUGAUCCCUCAAGAGAAUGGCAUCCUGCAGA
GCGAGUACGGCGGCGAGACAAUUCCUGGACCUGCCUUCAA
UCCCGCCAGCCAUCCUGCAUCUGCCCCUACAUCUAGCAGC
AGCAGCGCCUUCAGACCCGUGAUGCCUAGCAGACAGAUCG
UGGAACGGCAGCCCAGAAUGCUGGACUUCAGAGUCGAGUA
CCGGGACAGAAACGUGGACGUGGUGCUGGAAGAUACCUGC
ACCGUGGGCGAGAUCAAGCAGAUCCUGGAAAACGAGCUGC
AGAUCCCCGUGUCCAAGAUGCUGCUGAAAGGCUGGAAAAC
CGGCGACGUGGAAGAUAGCACCGUGCUGAAGUCCCUGCAU
CUCCCUAAGAACAACAGCCUGUACGUGCUGACCCCUGACC
UGCCUCCUCCAAGCUCUAGUUCUCAUGCUGGCGCCCUGCA
AGAGAGCCUGAACCAGAACUUCAUGCUGAUCAUCACCCAC
CGCGAGGUGCAGAGAGAGUAUAACCUGAACUUCAGCGGCA
GCAGCACCAUCCAAGAAGUGAAGCGGAACGUCUACGACCU
GACCAGCAUUCCUGUGCGGCACCAGCUUUGGGAAGGCUGG
CCUACAAGCGCCACCGACGAUUCUAUGUGUCUGGCCGAGA
GCGGCCUGAGCUACCCUUGUCACAGACUGACCGUGGGCAG
AAGAAGCAGCCCUGCUCAGACAAGAGAGCAGUCCGAGGAA
CAGAUCACCGACGUGCACAUGGUGUCCGAUAGCGACGGCG
ACGAUUUCGAGGAUGCCACCGAGUUUGGAGUGGACGACGG
CGAGGUUUUCGGCAUGGCUAGCAGCGCCCUGAGAAAGUCC
CCUAUGAUGCCCGAGAACGCCGAGAAUGAAGGCGACGCCC
UGCUGCAGUUUACCGCCGAGUUUAGCAGCAGAUACGGCGA
CUGUCACCCCGUGUUCUUCAUCGGAUCUCUGGAAGCCGCC
UUCCAAGAGGCCUUUUACGUGAAGGCCAGAGACAGAAAGC
UGCUGGCUAUCUAUCUGCACCACGACGAGAGCGUGCUGAC
AAACGUGUUCUGCAGCCAGAUGCUGUGCGCCGAGAGCAUC
GUGUCUUACCUGUCUCAGAAUUUCAUCACCUGGGCCUGGG
AUCUGACCAAGGACAGCAACAGAGCCCGGUUCCUGACCAU
GUGUAACCGGCACUUUGGCAGCGUGGUGGCCCAGACCAUC
AGAACCCAGAAAACCGAUCAGUUCCCUCUGUUCCUGAUCA
UUAUGGGCAAGCGCAGCAGCAACGAGGUGCUGAAUGUGAU
CCAGGGCAACACCACCGUGGACGAGCUGAUGAUGAGACUG
AUGGCCGCUAUGGAAAUCUUCACAGCCCAGCAGCAAGAAG
AUAUCAAGGACGAGGACGAGCGCGAGGCCCGCGAGAAUGU
GAAAAGAGAACAGGACGAAGCCUACCGGCUGAGCCUGGAA
GCUGACAGAGCCAAGAGAGAGGCCCACGAGAGAGAGAUGG
CCGAGCAGUUCAGACUCGAGCAGAUCCGCAAAGAGCAAGA
GGAAGAGAGAGAAGCCAUCCGGCUGUCCCUGGAACAAGCC
UUGCCUCCUGAGCCUAAAGAAGAGAACGCUGAGCCAGUGU
CCAAGCUGCGGAUCAGAACUCCUAGCGGCGAGUUCCUGGA
AAGACGGUUCCUGGCCUCCAACAAACUGCAGAUCGUGUUC
GACUUCGUGGCCUCUAAGGGCUUCCCCUGGGACGAGUACA
AGCUGCUGAGCACAUUCCCCAGACGGGACGUGACACAGCU
GGACCCUAACAAGAGCCUGCUGGAAGUGAAACUGUUUCCC
CAAGAGACACUGUUUCUCGAGGCCAAAGAGUGA

Hence, preferably the RNA construct comprises a sequence substantially as set out in SEQ ID No: 150, or a fragment or variant thereof.

In one embodiment, the at least one IMP may be a USP21 (NCBI Reference Sequence: NM_012475.5; UniProtKB—Q9UK80 (UBP21_HUMAN), or an orthologue thereof (Fan Y, Mao R, Yu Y, Liu S, Shi Z, Cheng J, Zhang H, An L, Zhao Y, Xu X, Chen Z, Kogiso M, Zhang D, Zhang H, Xhang P, Jung J U, LI, X, Xu G, Yang J. USP21 negatively regulates antiviral response by acting as a RIG-1 deubiquitinase. J Exp Med.; 211(2): 313-328). The USP21 is not dominant negative; it is the intact protein which acts as a negative regulator in antiviral responses through its ability to bind to and deubiquitinate RIG-I. Overexpression of USP21 inhibits RNA virus-induced RIG-I polyubiquitination and RIG-I-mediated interferon (IFN) signalling. One embodiment of the USP21 is provided a SEQ ID No: 166, as follows:

[SEQ ID No: 166]
MPQASEHRLGRTREPPVNIQPRVGSKLPFAPRARSKERRN
PASGPNPMLRPLPPRPGLPDERLKKLELGRGRTSGPRPRG
PLRADHGVPLPGSPPPTVALPLPSRTNLARSKSVSSGDLR
PMGIALGGHRGTGELGAALSRLALRPEPPTLRRSTSLRRL
GGFPGPPTLFSIRTEPPASHGSFHMISARSSEPFYSDDKM
AHHTLLLGSGHVGLRNLGNTCFLNAVLQCLSSTRPLRDFC
LRRDFRQEVPGGGRAQELTEAFADVIGALWHPDSCEAVNP
TRFRAVFQKYVPSFSGYSQQDAQEFLKLLMERLHLEINRR
GRRAPPILANGPVPSPPRRGGALLEEPELSDDDRANLMWK
RYLEREDSKIVDLFVGQLKSCLKCQACGYRSTTFEVFCDL
SLPIPKKGFAGGKVSLRDCFNLFTKEEELESENAPVCDRC
RQKTRSTKKLTVQRFPRILVLHLNRFSASRGSIKKSSVGV
DFPLQRLSLGDFASDKAGSPVYQLYALCNHSGSVHYGHYT
ALCRCQTGWHVYNDSRVSPVSENQVASSEGYVLFYQLMQE
PPRCL

Therefore, preferably the RNA construct of the first aspect comprises a nucleotide sequence which encodes an amino acid sequence substantially as set out in SEQ ID No: 166, or a variant or fragment thereof.

In one embodiment, the USP21 polypeptide is encoded by the DNA nucleotide sequence of SEQ ID No: 167, as follows:

[SEQ ID No: 167]
ATGCCCCAGGCCTCTGAGCACCGCCTGGGCCGTACCCGAG
AGCCACCTGTTAATATCCAGCCCCGAGTGGGATCCAAGCT
ACCATTTGCCCCCAGGGCCCGCAGCAAGGAGCGCAGAAAC
CCAGCCTCTGGGCCAAACCCCATGTTACGACCTCTGCCTC
CCCGGCCAGGTCTGCCTGATGAACGGCTCAAGAAACTGGA
GCTGGGACGGGGACGGACCTCAGGCCCTCGTCCCAGAGGC
CCCCTTCGAGCAGATCATGGGGTTCCCCTGCCTGGCTCAC
CACCCCCAACAGTGGCTTTGCCTCTCCCATCTCGGACCAA
CTTAGCCCGTTCCAAGTCTGTGAGCAGTGGGGACTTGCGT
CCAATGGGGATTGCCTTGGGAGGGCACCGTGGCACCGGAG
AGCTTGGGGCTGCACTGAGCCGCTTGGCCCTCCGGCCTGA
GCCACCCACTTTGAGACGTAGCACTTCTCTCCGCCGCCTA
GGGGGCTTTCCTGGACCCCCTACCCTGTTCAGCATACGGA
CAGAGCCCCCTGCTTCCCATGGCTCCTTCCACATGATATC
CGCCCGGTCCTCTGAGCCTTTCTACTCTGATGACAAGATG
GCTCATCACACACTCCTTCTGGGCTCTGGTCATGTTGGCC
TTCGAAACCTGGGAAACACGTGCTTCCTGAATGCTGTGCT
GCAGTGTCTGAGCAGCACTCGACCTCTTCGGGACTTCTGT
CTGAGAAGGGACTTCCGGCAAGAGGTGCCTGGAGGAGGCC
GAGCCCAAGAGCTCACTGAAGCCTTTGCAGATGTGATTGG
TGCCCTCTGGCACCCTGACTCCTGCGAAGCTGTGAATCCT
ACTCGATTCCGAGCTGTCTTCCAGAAATATGTTCCCTCCT
TCTCTGGATACAGCCAGCAGGATGCCCAAGAGTTCCTGAA
GCTCCTCATGGAGCGGCTACACCTTGAAATCAACCGCCGA
GGCCGCCGGGCTCCACCGATACTTGCCAATGGTCCAGTTC
CCTCTCCACCCCGCCGAGGAGGGGCTCTGCTAGAAGAACC
TGAGTTAAGTGATGATGACCGAGCCAACCTAATGTGGAAA
CGTTACCTGGAGCGAGAGGACAGCAAGATTGTGGACCTGT
TTGTGGGCCAGTTGAAAAGTTGTCTCAAGTGCCAGGCCTG
TGGGTATCGCTCCACGACCTTCGAGGTTTTTTGTGACCTG
TCCCTGCCCATCCCCAAGAAAGGATTTGCTGGGGGCAAGG
TGTCTCTGCGGGATTGTTTCAACCTTTTCACTAAGGAAGA
AGAGCTAGAGTCGGAGAATGCCCCAGTGTGTGACCGATGT
CGGCAGAAAACTCGAAGTACCAAAAAGTTGACAGTACAAA
GATTCCCTCGAATCCTCGTGCTCCATCTGAATCGATTTTC
TGCCTCCCGAGGCTCCATCAAAAAAAGTTCAGTAGGTGTA
GACTTTCCACTGCAGCGACTGAGCCTAGGGGACTTTGCCA
GTGACAAAGCCGGAAGTCCTGTATACCAGCTGTATGCCCT
TTGCAACCACTCAGGCAGCGTCCACTATGGCCACTACACA
GCCCTGTGCCGGTGCCAGACTGGTTGGCATGTCTACAATG
ACTCTCGTGTCTCCCCTGTCAGTGAAAACCAGGTGGCATC
CAGCGAGGGCTACGTGCTGTTCTACCAACTGATGCAGGAG
CCACCCCGGTGCCTG

Accordingly, preferably the USP21 polypeptide is encoded by the DNA nucleotide sequence substantially as set out in SEQ ID NO: 167, or a variant or fragment thereof.

Thus, the RNA construct may comprise an RNA nucleotide sequence of SEQ ID No: 168, as follows:

[SEQ ID No: 168]
AUGCCCCAGGCCUCUGAGCACCGCCUGGGCCGUACCCGAG
AGCCACCUGUUAAUAUCCAGCCCCGAGUGGGAUCCAAGCU
ACCAUUUGCCCCCAGGGCCCGCAGCAAGGAGCGCAGAAAC
CCAGCCUCUGGGCCAAACCCCAUGUUACGACCUCUGCCUC
CCCGGCCAGGUCUGCCUGAUGAACGGCUCAAGAAACUGGA
GCUGGGACGGGGACGGACCUCAGGCCCUCGUCCCAGAGGC
CCCCUUCGAGCAGAUCAUGGGGUUCCCCUGCCUGGCUCAC
CACCCCCAACAGUGGCUUUGCCUCUCCCAUCUCGGACCAA
CUUAGCCCGUUCCAAGUCUGUGAGCAGUGGGGACUUGCGU
CCAAUGGGGAUUGCCUUGGGAGGGCACCGUGGCACCGGAG
AGCUUGGGGCUGCACUGAGCCGCUUGGCCCUCCGGCCUGA
GCCACCCACUUUGAGACGUAGCACUUCUCUCCGCCGCCUA
GGGGGCUUUCCUGGACCCCCUACCCUGUUCAGCAUACGGA
CAGAGCCCCCUGCUUCCCAUGGCUCCUUCCACAUGAUAUC
CGCCCGGUCCUCUGAGCCUUUCUACUCUGAUGACAAGAUG
GCUCAUCACACACUCCUUCUGGGCUCUGGUCAUGUUGGCC
UUCGAAACCUGGGAAACACGUGCUUCCUGAAUGCUGUGCU
GCAGUGUCUGAGCAGCACUCGACCUCUUCGGGACUUCUGU
CUGAGAAGGGACUUCCGGCAAGAGGUGCCUGGAGGAGGCC
GAGCCCAAGAGCUCACUGAAGCCUUUGCAGAUGUGAUUGG
UGCCCUCUGGCACCCUGACUCCUGCGAAGCUGUGAAUCCU
ACUCGAUUCCGAGCUGUCUUCCAGAAAUAUGUUCCCUCCU
UCUCUGGAUACAGCCAGCAGGAUGCCCAAGAGUUCCUGAA
GCUCCUCAUGGAGCGGCUACACCUUGAAAUCAACCGCCGA
GGCCGCCGGGCUCCACCGAUACUUGCCAAUGGUCCAGUUC
CCUCUCCACCCCGCCGAGGAGGGGCUCUGCUAGAAGAACC
UGAGUUAAGUGAUGAUGACCGAGCCAACCUAAUGUGGAAA
CGUUACCUGGAGCGAGAGGACAGCAAGAUUGUGGACCUGU
UUGUGGGCCAGUUGAAAAGUUGUCUCAAGUGCCAGGCCUG
UGGGUAUCGCUCCACGACCUUCGAGGUUUUUUGUGACCUG
UCCCUGCCCAUCCCCAAGAAAGGAUUUGCUGGGGGCAAGG
UGUCUCUGCGGGAUUGUUUCAACCUUUUCACUAAGGAAGA
AGAGCUAGAGUCGGAGAAUGCCCCAGUGUGUGACCGAUGU
CGGCAGAAAACUCGAAGUACCAAAAAGUUGACAGUACAAA
GAUUCCCUCGAAUCCUCGUGCUCCAUCUGAAUCGAUUUUC
UGCCUCCCGAGGCUCCAUCAAAAAAAGUUCAGUAGGUGUA
GACUUUCCACUGCAGCGACUGAGCCUAGGGGACUUUGCCA
GUGACAAAGCCGGAAGUCCUGUAUACCAGCUGUAUGCCCU
UUGCAACCACUCAGGCAGCGUCCACUAUGGCCACUACACA
GCCCUGUGCCGGUGCCAGACUGGUUGGCAUGUCUACAAUG
ACUCUCGUGUCUCCCCUGUCAGUGAAAACCAGGUGGCAUC
CAGCGAGGGCUACGUGCUGUUCUACCAACUGAUGCAGGAG
CCACCCCGGUGCCUG

Therefore, preferably the RNA construct comprises an RNA nucleotide sequence substantially as set out in SEQ ID No: 168, or a variant or fragment thereof.

The inventors then subjected the protein sequence of SEQ ID No: 166 to codon optimisation for human expression, and one embodiment of the codon optimised 50 nucleic acid (DNA) sequence that includes a start (ATG) and a stop (TGA) codon is provided herein as SEQ ID No: 169, as follows:

[SEQ ID No: 169]
ATGCCTCAGGCCTCTGAGCACAGACTGGGCAGAACCAGAG
AACCTCCTGTGAACATCCAGCCTAGAGTGGGCAGCAAGCT
GCCCTTCGCTCCTAGAGCCAGAAGCAAAGAGCGGAGAAAC
CCTGCCAGCGGACCCAATCCTATGCTGAGGCCTTTGCCTC
CTAGACCTGGCCTGCCTGACGAGAGACTGAAGAAGCTGGA
ACTCGGCAGAGGCAGAACAAGCGGCCCTAGACCTAGAGGA
CCTCTGAGAGCTGATCACGGCGTTCCACTGCCTGGAAGCC
CTCCACCTACAGTTGCTCTGCCACTGCCTAGCAGGACCAA
CCTGGCCAGATCTAAGAGCGTGTCCAGCGGCGATCTGCGG
CCTATGGGAATTGCCCTCGGAGGCCATAGAGGAACAGGCG
AACTTGGAGCCGCTCTGAGCAGACTGGCCCTCAGACCTGA
ACCTCCTACACTGAGAAGAAGCACCAGCCTGAGAAGGCTC
GGCGGCTTTCCTGGACCACCAACACTGTTCAGCATCCGGA
CAGAGCCTCCAGCCAGCCACGGCAGCTTTCACATGATCAG
CGCCAGATCCAGCGAGCCCTTCTACAGCGACGACAAGATG
GCCCACCACACACTGCTGCTCGGCTCTGGACATGTGGGCC
TGAGAAACCTGGGCAATACCTGCTTCCTGAATGCCGTGCT
GCAGTGCCTGAGCAGCACAAGACCCCTGAGAGACTTCTGC
CTGCGGCGGGACTTTAGACAAGAAGTGCCTGGCGGAGGCA
GAGCCCAAGAACTGACAGAGGCTTTCGCCGATGTGATCGG
AGCCCTGTGGCACCCTGATTCTTGCGAGGCCGTGAATCCC
ACCAGATTCCGGGCCGTGTTCCAGAAATACGTGCCCAGCT
TTAGCGGCTACAGCCAGCAGGATGCCCAAGAGTTCCTGAA
GCTGCTGATGGAACGGCTGCACCTGGAAATCAACAGAAGA
GGCAGACGGGCCCCTCCTATCCTGGCTAATGGACCTGTTC
CTAGTCCTCCTAGAAGAGGCGGCGCTCTGCTGGAAGAACC
TGAGCTGAGCGACGACGACAGAGCCAACCTGATGTGGAAG
AGATACCTGGAACGCGAGGACAGCAAGATCGTGGATCTGT
TCGTGGGCCAGCTGAAGTCCTGCCTGAAGTGTCAGGCCTG
TGGCTACAGGTCCACCACCTTCGAGGTGTTCTGCGACCTG
TCTCTGCCCATTCCTAAGAAGGGCTTTGCCGGCGGAAAGG
TGTCCCTGAGGGACTGCTTCAACCTGTTCACCAAAGAGGA
AGAACTCGAGAGCGAGAACGCCCCTGTGTGCGACAGATGC
CGGCAGAAAACCCGGTCCACCAAGAAACTGACCGTGCAGC
GGTTCCCCAGAATCCTGGTGCTGCATCTGAACAGATTCTC
CGCCAGCCGGGGCAGCATCAAGAAAAGCTCTGTGGGCGTC
GACTTCCCACTGCAGCGACTGAGCCTGGGCGATTTCGCCT
CTGATAAGGCCGGCTCTCCTGTGTACCAGCTGTACGCCCT
GTGTAACCACAGCGGCTCTGTGCACTACGGCCACTACACC
GCTCTGTGTAGATGCCAGACAGGCTGGCACGTGTACAACG
ACAGCAGAGTGTCCCCTGTGTCCGAGAATCAGGTGGCCAG
CTCTGAGGGCTACGTGCTGTTCTACCAGCTGATGCAAGAG
CCTCCTCGGTGCCTGTGA

Hence, preferably the RNA construct is encoded by a DNA sequence substantially as set out in SEQ ID No: 169, or a fragment or variant thereof.

In an embodiment, the RNA sequence corresponding to the codon optimised DNA sequence of SEQ ID No: 169 that includes a start (AUG) and a stop (UGA) codon is provided herein as SEQ ID No: 170, as follows:

[SEQ ID No: 170]
AUGCCUCAGGCCUCUGAGCACAGACUGGGCAGAACCAGAG
AACCUCCUGUGAACAUCCAGCCUAGAGUGGGCAGCAAGCU
GCCCUUCGCUCCUAGAGCCAGAAGCAAAGAGCGGAGAAAC
CCUGCCAGCGGACCCAAUCCUAUGCUGAGGCCUUUGCCUC
CUAGACCUGGCCUGCCUGACGAGAGACUGAAGAAGCUGGA
ACUCGGCAGAGGCAGAACAAGCGGCCCUAGACCUAGAGGA
CCUCUGAGAGCUGAUCACGGCGUUCCACUGCCUGGAAGCC
CUCCACCUACAGUUGCUCUGCCACUGCCUAGCAGGACCAA
CCUGGCCAGAUCUAAGAGCGUGUCCAGCGGCGAUCUGCGG
CCUAUGGGAAUUGCCCUCGGAGGCCAUAGAGGAACAGGCG
AACUUGGAGCCGCUCUGAGCAGACUGGCCCUCAGACCUGA
ACCUCCUACACUGAGAAGAAGCACCAGCCUGAGAAGGCUC
GGCGGCUUUCCUGGACCACCAACACUGUUCAGCAUCCGGA
CAGAGCCUCCAGCCAGCCACGGCAGCUUUCACAUGAUCAG
CGCCAGAUCCAGCGAGCCCUUCUACAGCGACGACAAGAUG
GCCCACCACACACUGCUGCUCGGCUCUGGACAUGUGGGCC
UGAGAAACCUGGGCAAUACCUGCUUCCUGAAUGCCGUGCU
GCAGUGCCUGAGCAGCACAAGACCCCUGAGAGACUUCUGC
CUGCGGCGGGACUUUAGACAAGAAGUGCCUGGCGGAGGCA
GAGCCCAAGAACUGACAGAGGCUUUCGCCGAUGUGAUCGG
AGCCCUGUGGCACCCUGAUUCUUGCGAGGCCGUGAAUCCC
ACCAGAUUCCGGGCCGUGUUCCAGAAAUACGUGCCCAGCU
UUAGCGGCUACAGCCAGCAGGAUGCCCAAGAGUUCCUGAA
GCUGCUGAUGGAACGGCUGCACCUGGAAAUCAACAGAAGA
GGCAGACGGGCCCCUCCUAUCCUGGCUAAUGGACCUGUUC
CUAGUCCUCCUAGAAGAGGCGGCGCUCUGCUGGAAGAACC
UGAGCUGAGCGACGACGACAGAGCCAACCUGAUGUGGAAG
AGAUACCUGGAACGCGAGGACAGCAAGAUCGUGGAUCUGU
UCGUGGGCCAGCUGAAGUCCUGCCUGAAGUGUCAGGCCUG
UGGCUACAGGUCCACCACCUUCGAGGUGUUCUGCGACCUG
UCUCUGCCCAUUCCUAAGAAGGGCUUUGCCGGCGGAAAGG
UGUCCCUGAGGGACUGCUUCAACCUGUUCACCAAAGAGGA
AGAACUCGAGAGCGAGAACGCCCCUGUGUGCGACAGAUGC
CGGCAGAAAACCCGGUCCACCAAGAAACUGACCGUGCAGC
GGUUCCCCAGAAUCCUGGUGCUGCAUCUGAACAGAUUCUC
CGCCAGCCGGGGCAGCAUCAAGAAAAGCUCUGUGGGCGUC
GACUUCCCACUGCAGCGACUGAGCCUGGGCGAUUUCGCCU
CUGAUAAGGCCGGCUCUCCUGUGUACCAGCUGUACGCCCU
GUGUAACCACAGCGGCUCUGUGCACUACGGCCACUACACC
GCUCUGUGUAGAUGCCAGACAGGCUGGCACGUGUACAACG
ACAGCAGAGUGUCCCCUGUGUCCGAGAAUCAGGUGGCCAG
CUCUGAGGGCUACGUGCUGUUCUACCAGCUGAUGCAAGAG
CCUCCUCGGUGCCUGUGA

Hence, preferably the RNA construct comprises a sequence substantially as set out in SEQ ID No: 170, or a fragment or variant thereof.

In one embodiment, the at least one IMP may be a USP27 (1-438) (NCBI Reference Sequence: NM_001145073.3; UniProtKB—A6NNY8 (UBP27_HUMAN), or an orthologue thereof. The USP27 is not dominant negative; it is the intact protein which acts as a negative regulator in antiviral responses through its ability to bind to and deubiquitinate RIG-I. Overexpression of USP27 inhibits RNA virus-induced RIG-I polyubiquitination and RIG-I-mediated pathways leading to IFN production. One embodiment of the USP27 form is represented herein as SEQ ID No:171, as follows:

[SEQ ID No: 171]
MCKDYVYDKDIEQIAKEEQGEALKLQASTSTEVSHQQCSV
PGLGEKFPTWETTKPELELLGHNPRRRRITSSFTIGLRGL
INLGNTCFMNCIVQALTHTPILRDFFLSDRHRCEMPSPEL
CLVCEMSSLFRELYSGNPSPHVPYKLLHLVWIHARHLAGY
RQQDAHEFLIAALDVLHRHCKGDDVGKAANNPNHCNCIID
QIFTGGLQSDVTCQACHGVSTTIDPCWDISLDLPGSCTSF
WPMSPGRESSVNGESHIPGITTLTDCLRRFTRPEHLGSSA
KIKCGSCQSYQESTKQLTMNKLPVVACFHFKRFEHSAKQR
RKITTYISFPLELDMTPFMASSKESRMNGQLQLPTNSGNN
ENKYSLFAVVNHQGTLESGHYTSFIRHHKDQWFKCDDAVI
TKASIKDVLDSEGYLLFYHKQVLEHESEKVKEMNTQAY

Therefore, preferably the RNA construct of the first aspect comprises a nucleotide sequence which encodes an amino acid sequence substantially as set out in SEQ ID No: 171, or a variant or fragment thereof.

In one embodiment, the USP27 polypeptide is encoded by the DNA nucleotide sequence of SEQ ID No: 172, as follows:

[SEQ ID No: 172]
ATGTGTAAGGACTATGTATATGACAAAGACATTGAGCAAA
TTGCCAAAGAAGAGCAAGGAGAAGCTTTGAAATTACAAGC
CTCCACCTCAACAGAGGTTTCTCACCAGCAGTGTTCAGTG
CCAGGCCTTGGTGAGAAATTCCCAACCTGGGAAACAACCA
AACCAGAATTAGAACTGCTGGGGCACAACCCGAGGAGAAG
AAGAATCACCTCCAGCTTTACGATCGGTTTAAGAGGACTC
ATCAATCTTGGCAACACGTGCTTTATGAACTGCATTGTCC
AGGCCCTCACCCACACGCCGATACTGAGAGATTTCTTTCT
CTCTGACAGGCACCGATGTGAGATGCCGAGTCCCGAGTTG
TGTCTGGTCTGTGAGATGTCGTCGCTGTTTCGGGAGTTGT
ATTCTGGAAACCCGTCTCCTCATGTGCCCTATAAGTTACT
GCACCTGGTGTGGATACATGCCCGCCATTTAGCAGGGTAC
AGGCAACAGGATGCCCACGAGTTCCTCATTGCAGCGTTAG
ATGTCCTGCACAGGCACTGCAAAGGTGATGATGTCGGGAA
GGCGGCCAACAATCCCAACCACTGTAACTGCATCATAGAC
CAAATCTTCACAGGTGGCCTGCAGTCTGATGTCACCTGTC
AAGCCTGCCATGGCGTCTCCACCACGATAGACCCATGCTG
GGACATTAGTTTGGACTTGCCTGGCTCTTGCACCTCCTTC
TGGCCCATGAGCCCAGGGAGGGAGAGCAGTGTGAACGGGG
AAAGCCACATACCAGGAATCACCACCCTCACGGACTGCTT
GCGGAGGTTTACGAGGCCAGAGCACTTAGGAAGCAGTGCC
AAAATCAAATGTGGTAGTTGCCAAAGCTACCAGGAATCTA
CCAAACAGCTCACAATGAATAAATTACCTGTCGTTGCCTG
TTTTCATTTCAAACGGTTTGAACATTCAGCGAAACAGAGG
CGCAAGATCACTACATACATTTCCTTTCCTCTGGAGCTGG
ATATGACGCCGTTTATGGCCTCAAGTAAAGAGAGCAGAAT
GAATGGACAATTGCAGCTGCCAACCAATAGTGGAAACAAC
GAAAATAAGTATTCCTTGTTTGCTGTGGTTAATCACCAAG
GAACCTTGGAGAGTGGCCACTATACCAGCTTCATCCGGCA
CCACAAGGACCAGTGGTTCAAGTGTGATGATGCCGTCATC
ACTAAGGCCAGTATTAAGGACGTACTGGACAGTGAAGGGT
ATTTACTGTTCTATCACAAACAGGTGCTAGAACATGAGTC
AGAAAAAGTGAAAGAAATGAACACACAAGCCTAC

Accordingly, preferably the USP27 polypeptide is encoded by the DNA nucleotide sequence substantially as set out in SEQ ID NO: 172, or a variant or fragment thereof.

Thus, the RNA construct may comprise an RNA nucleotide sequence of SEQ ID No: 173, as follows:

[SEQ ID No: 173]
AUGUGUAAGGACUAUGUAUAUGACAAAGACAUUGAGCAAA
UUGCCAAAGAAGAGCAAGGAGAAGCUUUGAAAUUACAAGC
CUCCACCUCAACAGAGGUUUCUCACCAGCAGUGUUCAGUG
CCAGGCCUUGGUGAGAAAUUCCCAACCUGGGAAACAACCA
AACCAGAAUUAGAACUGCUGGGGCACAACCCGAGGAGAAG
AAGAAUCACCUCCAGCUUUACGAUCGGUUUAAGAGGACUC
AUCAAUCUUGGCAACACGUGCUUUAUGAACUGCAUUGUCC
AGGCCCUCACCCACACGCCGAUACUGAGAGAUUUCUUUCU
CUCUGACAGGCACCGAUGUGAGAUGCCGAGUCCCGAGUUG
UGUCUGGUCUGUGAGAUGUCGUCGCUGUUUCGGGAGUUGU
AUUCUGGAAACCCGUCUCCUCAUGUGCCCUAUAAGUUACU
GCACCUGGUGUGGAUACAUGCCCGCCAUUUAGCAGGGUAC
AGGCAACAGGAUGCCCACGAGUUCCUCAUUGCAGCGUUAG
AUGUCCUGCACAGGCACUGCAAAGGUGAUGAUGUCGGGAA
GGCGGCCAACAAUCCCAACCACUGUAACUGCAUCAUAGAC
CAAAUCUUCACAGGUGGCCUGCAGUCUGAUGUCACCUGUC
AAGCCUGCCAUGGCGUCUCCACCACGAUAGACCCAUGCUG
GGACAUUAGUUUGGACUUGCCUGGCUCUUGCACCUCCUUC
UGGCCCAUGAGCCCAGGGAGGGAGAGCAGUGUGAACGGGG
AAAGCCACAUACCAGGAAUCACCACCCUCACGGACUGCUU
GCGGAGGUUUACGAGGCCAGAGCACUUAGGAAGCAGUGCC
AAAAUCAAAUGUGGUAGUUGCCAAAGCUACCAGGAAUCUA
CCAAACAGCUCACAAUGAAUAAAUUACCUGUCGUUGCCUG
UUUUCAUUUCAAACGGUUUGAACAUUCAGCGAAACAGAGG
CGCAAGAUCACUACAUACAUUUCCUUUCCUCUGGAGCUGG
AUAUGACGCCGUUUAUGGCCUCAAGUAAAGAGAGCAGAAU
GAAUGGACAAUUGCAGCUGCCAACCAAUAGUGGAAACAAC
GAAAAUAAGUAUUCCUUGUUUGCUGUGGUUAAUCACCAAG
GAACCUUGGAGAGUGGCCACUAUACCAGCUUCAUCCGGCA
CCACAAGGACCAGUGGUUCAAGUGUGAUGAUGCCGUCAUC
ACUAAGGCCAGUAUUAAGGACGUACUGGACAGUGAAGGGU
AUUUACUGUUCUAUCACAAACAGGUGCUAGAACAUGAGUC
AGAAAAAGUGAAAGAAAUGAACACACAAGCCUAC

Therefore, preferably the RNA construct comprises an RNA nucleotide sequence substantially as set out in SEQ ID No: 173, or a variant or fragment thereof.

The inventors then subjected the protein sequence of SEQ ID No: 171 to codon optimisation for human expression, and one embodiment of the codon optimised nucleic acid (DNA) sequence that includes a start (ATG) and a stop (TGA) codon is provided herein as SEQ ID No: 174, as follows:

[SEQ ID No: 174]
ATGTGCAAGGACTACGTGTACGACAAGGACATCGAGCAGA
TCGCCAAAGAGGAACAGGGCGAAGCCCTGAAGCTGCAGGC
CAGCACATCTACAGAGGTGTCCCACCAGCAGTGTAGCGTG
CCAGGACTGGGCGAGAAGTTCCCTACCTGGGAAACCACCA
AGCCTGAGCTGGAACTGCTGGGCCACAATCCTCGGCGGAG
AAGAATCACCAGCAGCTTCACCATCGGCCTGCGGGGCCTG
ATCAATCTGGGCAATACCTGCTTCATGAACTGCATCGTGC
AGGCCCTGACACACACCCCTATCCTGAGAGACTTCTTCCT
GTCCGACCGGCACAGATGCGAGATGCCTTCTCCAGAGCTG
TGCCTCGTGTGCGAGATGAGCAGCCTGTTCCGGGAACTGT
ACAGCGGCAACCCTTCTCCTCACGTGCCCTACAAACTGCT
GCACCTCGTGTGGATTCACGCCAGACACCTGGCCGGCTAC
AGACAGCAGGATGCCCACGAGTTTCTGATCGCCGCTCTGG
ACGTGCTGCACAGACACTGCAAAGGCGACGATGTGGGCAA
AGCCGCCAACAATCCCAACCACTGCAACTGCATCATCGAC
CAGATCTTCACAGGCGGCCTGCAGAGCGACGTTACCTGTC
AAGCTTGTCACGGCGTGTCCACCACCATCGATCCCTGCTG
GGATATCAGCCTGGATCTGCCTGGCAGCTGCACCAGCTTT
TGGCCTATGAGCCCTGGCAGAGAAAGCAGCGTGAACGGCG
AGTCTCACATCCCCGGCATCACCACACTGACCGACTGCCT
GCGGAGATTCACCAGACCTGAGCACCTGGGAAGCAGCGCC
AAGATCAAGTGTGGCTCCTGCCAGAGCTACCAAGAGAGCA
CCAAGCAGCTGACCATGAACAAGCTGCCTGTGGTGGCCTG
CTTCCACTTCAAGAGATTCGAGCACTCCGCCAAGCAGCGG
CGGAAGATCACAACCTACATCAGCTTCCCTCTGGAACTGG
ACATGACCCCTTTCATGGCCAGCAGCAAAGAAAGCCGGAT
GAACGGCCAGCTCCAGCTGCCTACAAATAGCGGCAACAAC
GAGAACAAGTACTCCCTGTTCGCCGTGGTCAACCACCAGG
GCACACTGGAAAGCGGCCACTACACCAGCTTCATCAGACA
CCACAAGGACCAGTGGTTCAAGTGCGACGACGCCGTGATC
ACCAAGGCCAGCATCAAGGATGTCCTGGACAGCGAGGGCT
ACCTGCTGTTCTACCACAAACAGGTGCTGGAACACGAGAG
CGAGAAAGTGAAAGAGATGAACACCCAGGCCTACTGA

Hence, preferably the RNA construct is encoded by a DNA sequence substantially as set out in SEQ ID No: 174, or a fragment or variant thereof.

In an embodiment, the RNA sequence corresponding to the codon optimised DNA sequence of SEQ ID No: 174 that includes a start (AUG) and a stop (UGA) codon is provided herein as SEQ ID No: 175, as follows:

[SEQ ID No: 175]
AUGUGCAAGGACUACGUGUACGACAAGGACAUCGAGCAGA
UCGCCAAAGAGGAACAGGGCGAAGCCCUGAAGCUGCAGGC
CAGCACAUCUACAGAGGUGUCCCACCAGCAGUGUAGCGUG
CCAGGACUGGGCGAGAAGUUCCCUACCUGGGAAACCACCA
AGCCUGAGCUGGAACUGCUGGGCCACAAUCCUCGGCGGAG
AAGAAUCACCAGCAGCUUCACCAUCGGCCUGCGGGGCCUG
AUCAAUCUGGGCAAUACCUGCUUCAUGAACUGCAUCGUGC
AGGCCCUGACACACACCCCUAUCCUGAGAGACUUCUUCCU
GUCCGACCGGCACAGAUGCGAGAUGCCUUCUCCAGAGCUG
UGCCUCGUGUGCGAGAUGAGCAGCCUGUUCCGGGAACUGU
ACAGCGGCAACCCUUCUCCUCACGUGCCCUACAAACUGCU
GCACCUCGUGUGGAUUCACGCCAGACACCUGGCCGGCUAC
AGACAGCAGGAUGCCCACGAGUUUCUGAUCGCCGCUCUGG
ACGUGCUGCACAGACACUGCAAAGGCGACGAUGUGGGCAA
AGCCGCCAACAAUCCCAACCACUGCAACUGCAUCAUCGAC
CAGAUCUUCACAGGCGGCCUGCAGAGCGACGUUACCUGUC
AAGCUUGUCACGGCGUGUCCACCACCAUCGAUCCCUGCUG
GGAUAUCAGCCUGGAUCUGCCUGGCAGCUGCACCAGCUUU
UGGCCUAUGAGCCCUGGCAGAGAAAGCAGCGUGAACGGCG
AGUCUCACAUCCCCGGCAUCACCACACUGACCGACUGCCU
GCGGAGAUUCACCAGACCUGAGCACCUGGGAAGCAGCGCC
AAGAUCAAGUGUGGCUCCUGCCAGAGCUACCAAGAGAGCA
CCAAGCAGCUGACCAUGAACAAGCUGCCUGUGGUGGCCUG
CUUCCACUUCAAGAGAUUCGAGCACUCCGCCAAGCAGCGG
CGGAAGAUCACAACCUACAUCAGCUUCCCUCUGGAACUGG
ACAUGACCCCUUUCAUGGCCAGCAGCAAAGAAAGCCGGAU
GAACGGCCAGCUCCAGCUGCCUACAAAUAGCGGCAACAAC
GAGAACAAGUACUCCCUGUUCGCCGUGGUCAACCACCAGG
GCACACUGGAAAGCGGCCACUACACCAGCUUCAUCAGACA
CCACAAGGACCAGUGGUUCAAGUGCGACGACGCCGUGAUC
ACCAAGGCCAGCAUCAAGGAUGUCCUGGACAGCGAGGGCU
ACCUGCUGUUCUACCACAAACAGGUGCUGGAACACGAGAG
CGAGAAAGUGAAAGAGAUGAACACCCAGGCCUACUGA

Hence, preferably the RNA construct comprises a sequence substantially as set out in SEQ ID No: 175, or a fragment or variant thereof.

In one embodiment, the at least one IMP may be a CYLD (NCBI Reference Sequence: NM_015247.3; UniProtKB—Q9NQC7 (CYLD_HUMAN), or an orthologue thereof (Friedman C S, O'Donell M A, Legarda-Addision D, Ng A, Cardenas W B, Young J S, Moran™, Basler C F, Komuro A, Horvath C M, Xavier R, Ting A T. The tumour suppressor CYLD is a negative regulator of RIG-I-mediated antiviral response. EMBO Rep. 2008; 9(9): 930-93. Ectopic expression of CYLD inhibits the IRF3 signalling pathway and IFN production is triggered by RIG-I. One embodiment of the CYLD is represented herein as SEQ ID No: 176, as follows:

[SEQ ID No: 176]
MSSGLWSQEKVTSPYWEERIFYLLLQECSVTDKQTQKLLKVPKGS
IGQYIQDRSVGHSRIPSAKGKKNQIGLKILEQPHAVLFVDEKDVV
EINEKFTELLLAITNCEERFSLFKNRNRLSKGLQIDVGCPVKVQL
RSGEEKFPGVVRERGPLLAERTVSGIFFGVELLEEGRGQGFTDGV
YQGKQLFQCDEDCGVFVALDKLELIEDDDTALESDYAGPGDTMQV
ELPPLEINSRVSLKVGETIESGTVIFCDVLPGKESLGYFVGVDMD
NPIGNWDGRFDGVQLCSFACVESTILLHINDIIPALSESVTQERR
PPKLAFMSRGVGDKGSSSHNKPKATGSTSDPGNRNRSELFYTLNG
SSVDSQPQSKSKNTWYIDEVAEDPAKSLTEISTDFDRSSPPLQPP
PVNSLTTENRFHSLPFSLIKMPNINGSIGHSPLSLSAQSVMEELN
TAPVQESPPLAMPPGNSHGLEVGSLAEVKENPPFYGVIRWIGQPP
GLNEVLAGLELEDECAGCTDGTFRGTRYFTCALKKALFVKLKSCR
PDSRFASLQPVSNQIERCNSLAFGGYLSEVVEENTPPKMEKEGLE
IMIGKKKGIQGHYNSCYLDSTLFCLFAFSSVLDTVLLRPKEKNDV
EYYSETQELLRTEIVNPLRIYGYVCATKIMKLRKILEKVEAASGF
TSEEKDPEEFLNILFHHILRVEPLLKIRSAGQKVQDCYFYQIFME
KNEKVGVPTIQQLLEWSFINSNLKFAEAPSCLIIQMPRFGKDFKL
FKKIFPSLELNITDLLEDTPRQCRICGGLAMYECRECYDDPDISA
GKIKQFCKTCNTQVHLHPKRLNHKYNPVSLPKDLPDWDWRHGCIP
CQNMELFAVLCIETSHYVAFVKYGKDDSAWLFFDSMADRDGGQNG
FNIPQVTPCPEVGEYLKMSLEDLHSLDSRRIQGCARRLLCDAYMC
MYQSPTMSLYK

Therefore, preferably the RNA construct of the first aspect comprises a nucleotide sequence which encodes an amino acid sequence substantially as set out in SEQ ID No: 176, or a variant or fragment thereof.

In one embodiment, the CYLD polypeptide is encoded by the DNA nucleotide sequence of SEQ ID No: 177, as follows:

[SEQ ID No: 177]
ATGAGTTCAGGCTTATGGAGCCAAGAAAAAGTCACTTCACCCTAC
TGGGAAGAGCGGATTTTTTACTTGCTTCTTCAAGAATGCAGCGTT
ACAGACAAACAAACACAAAAGCTCCTTAAAGTACCGAAGGGAAGT
ATAGGACAGTATATTCAAGATCGTTCTGTGGGGCATTCAAGGATT
CCTTCTGCAAAAGGCAAGAAAAATCAGATTGGATTAAAAATTCTA
GAGCAACCTCATGCAGTTCTCTTTGTTGATGAAAAGGATGTTGTA
GAGATAAATGAAAAGTTCACAGAGTTACTTTTGGCAATTACCAAT
TGTGAGGAGAGGTTCAGCCTGTTTAAAAACAGAAACAGACTAAGT
AAAGGCCTCCAAATAGACGTGGGCTGTCCTGTGAAAGTACAGCTG
AGATCTGGGGAAGAAAAATTTCCTGGAGTTGTACGCTTCAGAGGA
CCCCTGTTAGCAGAGAGGACAGTCTCCGGAATATTCTTTGGAGTT
GAATTGCTGGAAGAAGGTCGTGGTCAAGGTTTCACTGACGGGGTG
TACCAAGGGAAACAGCTTTTTCAGTGTGATGAAGATTGTGGCGTG
TTTGTTGCATTGGACAAGCTAGAACTCATAGAAGATGATGACACT
GCATTGGAAAGTGATTACGCAGGTCCTGGGGACACAATGCAGGTC
GAACTTCCTCCTTTGGAAATAAACTCCAGAGTTTCTTTGAAGGTT
GGAGAAACAATAGAATCTGGAACAGTTATATTCTGTGATGTTTTG
CCAGGAAAAGAAAGCTTAGGATATTTTGTTGGTGTGGACATGGAT
AACCCTATTGGCAACTGGGATGGAAGATTTGATGGAGTGCAGCTT
TGTAGTTTTGCGTGTGTTGAAAGTACAATTCTATTGCACATCAAT
GATATCATCCCAGCTTTATCAGAGAGTGTGACGCAGGAAAGGAGG
CCTCCCAAACTTGCCTTTATGTCAAGAGGTGTTGGGGACAAAGGT
TCATCCAGTCATAATAAACCAAAGGCTACAGGATCTACCTCAGAC
CCTGGAAATAGAAACAGATCTGAATTATTTTATACCTTAAATGGG
TCTTCTGTTGACTCACAACCACAATCCAAATCAAAAAATACATGG
TACATTGATGAAGTTGCAGAAGACCCTGCAAAATCTCTTACAGAG
ATATCTACAGACTTTGACCGTTCTTCACCACCACTCCAGCCTCCT
CCTGTGAACTCACTGACCACCGAGAACAGATTCCACTCTTTACCA
TTCAGTCTCACCAAGATGCCCAATACCAATGGAAGTATTGGCCAC
AGTCCACTTTCTCTGTCAGCCCAGTCTGTAATGGAAGAGCTAAAC
ACTGCACCCGTCCAAGAGAGTCCACCCTTGGCCATGCCTCCTGGG
AACTCACATGGTCTAGAAGTGGGCTCATTGGCTGAAGTTAAGGAG
AACCCTCCTTTCTATGGGGTAATCCGTTGGATCGGTCAGCCACCA
GGACTGAATGAAGTGCTCGCTGGACTGGAACTGGAAGATGAGTGT
GCAGGCTGTACGGATGGAACCTTCAGAGGCACTCGGTATTTCACC
TGTGCCCTGAAGAAGGCGCTGTTTGTGAAACTGAAGAGCTGCAGG
CCTGACTCTAGGTTTGCATCATTGCAGCCGGTTTCCAATCAGATT
GAGCGCTGTAACTCTTTAGCATTTGGAGGCTACTTAAGTGAAGTA
GTAGAAGAAAATACTCCACCAAAAATGGAAAAAGAAGGCTTGGAG
ATAATGATTGGGAAGAAGAAAGGCATCCAGGGTCATTACAATTCT
TGTTACTTAGACTCAACCTTATTCTGCTTATTTGCTTTTAGTTCT
GTTCTGGACACTGTGTTACTTAGACCCAAAGAAAAGAACGATGTA
GAATATTATAGTGAAACCCAAGAGCTACTGAGGACAGAAATTGTT
AATCCTCTGAGAATATATGGATATGTGTGTGCCACAAAAATTATG
AAACTGAGGAAAATACTTGAAAAGGTGGAGGCTGCATCAGGATTT
ACCTCTGAAGAAAAAGATCCTGAGGAATTCTTGAATATTCTGTTT
CATCATATTTTAAGGGTAGAACCTTTGCTAAAAATAAGATCAGCA
GGTCAAAAGGTACAAGATTGTTACTTCTATCAAATTTTTATGGAA
AAAAATGAGAAAGTTGGCGTTCCCACAATTCAGCAGTTGTTAGAA
TGGTCTTTTATCAACAGTAACCTGAAATTTGCAGAGGCACCATCA
TGTCTGATTATTCAGATGCCTCGATTTGGAAAAGACTTTAAACTA
TTTAAAAAAATTTTTCCTTCTCTGGAATTAAATATAACAGATTTA
CTTGAAGACACTCCCAGACAGTGCCGGATATGTGGAGGGCTTGCA
ATGTATGAGTGTAGAGAATGCTACGACGATCCGGACATCTCAGCT
GGAAAAATCAAGCAGTTTTGTAAAACCTGCAACACTCAAGTCCAC
CTTCATCCGAAGAGGCTGAATCATAAATATAACCCAGTGTCACTT
CCCAAAGACTTACCCGACTGGGACTGGAGACACGGCTGCATCCCT
TGCCAGAATATGGAGTTATTTGCTGTTCTCTGCATAGAAACAAGC
CACTATGTTGCTTTTGTGAAGTATGGGAAGGACGATTCTGCCTGG
CTCTTCTTTGACAGCATGGCCGATCGGGATGGTGGTCAGAATGGC
TTCAACATTCCTCAAGTCACCCCATGCCCAGAAGTAGGAGAGTAC
TTGAAGATGTCTCTGGAAGACCTGCATTCCTTGGACTCCAGGAGA
ATCCAAGGCTGTGCACGAAGACTGCTTTGTGATGCATATATGTGC
ATGTACCAGAGTCCAACAATGAGTTTGTACAAA

Accordingly, preferably the CYLD polypeptide is encoded by the DNA nucleotide sequence substantially as set out in SEQ ID NO: 177, or a variant or fragment thereof.

Thus, the RNA construct may comprise an RNA nucleotide sequence of SEQ ID No: 178, as follows:

[SEQ ID No: 178]
AUGAGUUCAGGCUUAUGGAGCCAAGAAAAAGUCACUUCACCCUAC
UGGGAAGAGCGGAUUUUUUACUUGCUUCUUCAAGAAUGCAGCGUU
ACAGACAAACAAACACAAAAGCUCCUUAAAGUACCGAAGGGAAGU
AUAGGACAGUAUAUUCAAGAUCGUUCUGUGGGGCAUUCAAGGAUU
CCUUCUGCAAAAGGCAAGAAAAAUCAGAUUGGAUUAAAAAUUCUA
GAGCAACCUCAUGCAGUUCUCUUUGUUGAUGAAAAGGAUGUUGUA
GAGAUAAAUGAAAAGUUCACAGAGUUACUUUUGGCAAUUACCAAU
UGUGAGGAGAGGUUCAGCCUGUUUAAAAACAGAAACAGACUAAGU
AAAGGCCUCCAAAUAGACGUGGGCUGUCCUGUGAAAGUACAGCUG
AGAUCUGGGGAAGAAAAAUUUCCUGGAGUUGUACGCUUCAGAGGA
CCCCUGUUAGCAGAGAGGACAGUCUCCGGAAUAUUCUUUGGAGUU
GAAUUGCUGGAAGAAGGUCGUGGUCAAGGUUUCACUGACGGGGUG
UACCAAGGGAAACAGCUUUUUCAGUGUGAUGAAGAUUGUGGCGUG
UUUGUUGCAUUGGACAAGCUAGAACUCAUAGAAGAUGAUGACACU
GCAUUGGAAAGUGAUUACGCAGGUCCUGGGGACACAAUGCAGGUC
GAACUUCCUCCUUUGGAAAUAAACUCCAGAGUUUCUUUGAAGGUU
GGAGAAACAAUAGAAUCUGGAACAGUUAUAUUCUGUGAUGUUUUG
CCAGGAAAAGAAAGCUUAGGAUAUUUUGUUGGUGUGGACAUGGAU
AACCCUAUUGGCAACUGGGAUGGAAGAUUUGAUGGAGUGCAGCUU
UGUAGUUUUGCGUGUGUUGAAAGUACAAUUCUAUUGCACAUCAAU
GAUAUCAUCCCAGCUUUAUCAGAGAGUGUGACGCAGGAAAGGAGG
CCUCCCAAACUUGCCUUUAUGUCAAGAGGUGUUGGGGACAAAGGU
UCAUCCAGUCAUAAUAAACCAAAGGCUACAGGAUCUACCUCAGAC
CCUGGAAAUAGAAACAGAUCUGAAUUAUUUUAUACCUUAAAUGGG
UCUUCUGUUGACUCACAACCACAAUCCAAAUCAAAAAAUACAUGG
UACAUUGAUGAAGUUGCAGAAGACCCUGCAAAAUCUCUUACAGAG
AUAUCUACAGACUUUGACCGUUCUUCACCACCACUCCAGCCUCCU
CCUGUGAACUCACUGACCACCGAGAACAGAUUCCACUCUUUACCA
UUCAGUCUCACCAAGAUGCCCAAUACCAAUGGAAGUAUUGGCCAC
AGUCCACUUUCUCUGUCAGCCCAGUCUGUAAUGGAAGAGCUAAAC
ACUGCACCCGUCCAAGAGAGUCCACCCUUGGCCAUGCCUCCUGGG
AACUCACAUGGUCUAGAAGUGGGCUCAUUGGCUGAAGUUAAGGAG
AACCCUCCUUUCUAUGGGGUAAUCCGUUGGAUCGGUCAGCCACCA
GGACUGAAUGAAGUGCUCGCUGGACUGGAACUGGAAGAUGAGUGU
GCAGGCUGUACGGAUGGAACCUUCAGAGGCACUCGGUAUUUCACC
UGUGCCCUGAAGAAGGCGCUGUUUGUGAAACUGAAGAGCUGCAGG
CCUGACUCUAGGUUUGCAUCAUUGCAGCCGGUUUCCAAUCAGAUU
GAGCGCUGUAACUCUUUAGCAUUUGGAGGCUACUUAAGUGAAGUA
GUAGAAGAAAAUACUCCACCAAAAAUGGAAAAAGAAGGCUUGGAG
AUAAUGAUUGGGAAGAAGAAAGGCAUCCAGGGUCAUUACAAUUCU
UGUUACUUAGACUCAACCUUAUUCUGCUUAUUUGCUUUUAGUUCU
GUUCUGGACACUGUGUUACUUAGACCCAAAGAAAAGAACGAUGUA
GAAUAUUAUAGUGAAACCCAAGAGCUACUGAGGACAGAAAUUGUU
AAUCCUCUGAGAAUAUAUGGAUAUGUGUGUGCCACAAAAAUUAUG
AAACUGAGGAAAAUACUUGAAAAGGUGGAGGCUGCAUCAGGAUUU
ACCUCUGAAGAAAAAGAUCCUGAGGAAUUCUUGAAUAUUCUGUUU
CAUCAUAUUUUAAGGGUAGAACCUUUGCUAAAAAUAAGAUCAGCA
GGUCAAAAGGUACAAGAUUGUUACUUCUAUCAAAUUUUUAUGGAA
AAAAAUGAGAAAGUUGGCGUUCCCACAAUUCAGCAGUUGUUAGAA
UGGUCUUUUAUCAACAGUAACCUGAAAUUUGCAGAGGCACCAUCA
UGUCUGAUUAUUCAGAUGCCUCGAUUUGGAAAAGACUUUAAACUA
UUUAAAAAAAUUUUUCCUUCUCUGGAAUUAAAUAUAACAGAUUUA
CUUGAAGACACUCCCAGACAGUGCCGGAUAUGUGGAGGGCUUGCA
AUGUAUGAGUGUAGAGAAUGCUACGACGAUCCGGACAUCUCAGCU
GGAAAAAUCAAGCAGUUUUGUAAAACCUGCAACACUCAAGUCCAC
CUUCAUCCGAAGAGGCUGAAUCAUAAAUAUAACCCAGUGUCACUU
CCCAAAGACUUACCCGACUGGGACUGGAGACACGGCUGCAUCCCU
UGCCAGAAUAUGGAGUUAUUUGCUGUUCUCUGCAUAGAAACAAGC
CACUAUGUUGCUUUUGUGAAGUAUGGGAAGGACGAUUCUGCCUGG
CUCUUCUUUGACAGCAUGGCCGAUCGGGAUGGUGGUCAGAAUGGC
UUCAACAUUCCUCAAGUCACCCCAUGCCCAGAAGUAGGAGAGUAC
UUGAAGAUGUCUCUGGAAGACCUGCAUUCCUUGGACUCCAGGAGA
AUCCAAGGCUGUGCACGAAGACUGCUUUGUGAUGCAUAUAUGUGC
AUGUACCAGAGUCCAACAAUGAGUUUGUACAAA

Therefore, preferably the RNA construct comprises an RNA nucleotide sequence substantially as set out in SEQ ID No: 178, or a variant or fragment thereof.

The inventors then subjected the protein sequence of SEQ ID No: 176 to codon optimisation for human expression, and one embodiment of the codon optimised nucleic acid (DNA) sequence that includes a start (ATG) and a stop (TGA) codon is provided herein as SEQ ID No: 179, as follows:

[SEQ ID No: 179]
ATGTCTAGCGGCCTGTGGTCCCAAGAGAAAGTGACAAGCCCCTAC
TGGGAAGAGAGGATCTTCTACCTGCTGCTGCAAGAGTGCAGCGTG
ACCGACAAGCAGACCCAGAAACTGCTGAAGGTGCCCAAGGGCAGC
ATCGGCCAGTACATCCAGGATAGAAGCGTGGGCCACAGCAGAATC
CCTAGCGCCAAGGGCAAGAAGAACCAGATCGGCCTGAAGATCCTG
GAACAGCCTCACGCCGTGCTGTTCGTGGACGAGAAGGACGTGGTG
GAAATCAACGAGAAGTTCACCGAGCTGCTGCTGGCCATCACCAAC
TGCGAGGAACGGTTCAGCCTGTTCAAGAACCGGAACCGGCTGAGC
AAGGGCCTGCAGATCGATGTGGGATGCCCTGTGAAGGTGCAGCTG
AGAAGCGGCGAAGAGAAGTTCCCTGGCGTCGTGCGGTTTAGAGGA
CCTCTGCTGGCCGAGAGAACCGTGTCCGGCATCTTCTTTGGCGTG
GAACTGCTGGAAGAAGGCAGAGGCCAGGGCTTTACCGATGGCGTG
TACCAGGGCAAGCAGCTGTTTCAGTGCGACGAGGATTGCGGCGTG
TTCGTGGCCCTGGATAAGCTGGAACTGATCGAGGACGACGACACA
GCCCTGGAAAGCGATTATGCCGGACCTGGCGATACCATGCAGGTC
GAACTGCCTCCACTCGAGATCAACAGCCGGGTGTCCCTGAAAGTG
GGCGAGACAATCGAGAGCGGCACCGTGATCTTTTGCGACGTGCTG
CCTGGCAAAGAGTCCCTGGGCTATTTTGTGGGCGTCGACATGGAC
AACCCCATCGGCAATTGGGACGGCAGATTTGACGGCGTGCAGCTG
TGCAGCTTCGCCTGTGTGGAAAGCACCATCCTGCTGCACATCAAC
GACATCATCCCCGCTCTGAGCGAGAGCGTGACCCAAGAAAGACGG
CCTCCTAAGCTGGCCTTCATGTCTAGAGGCGTGGGCGATAAGGGC
AGCTCCAGCCACAACAAGCCTAAGGCCACAGGCTCCACAAGCGAC
CCCGGCAACAGAAACAGAAGCGAGCTGTTCTACACCCTGAACGGC
AGCAGCGTGGACAGCCAGCCTCAGAGCAAGAGCAAGAACACCTGG
TACATCGACGAGGTGGCCGAGGATCCTGCCAAGAGCCTGACAGAG
ATCAGCACCGACTTCGACAGAAGCAGCCCTCCACTGCAGCCTCCA
CCTGTGAATAGCCTGACCACCGAGAACAGATTCCACAGCCTGCCT
TTCAGCCTGACTAAGATGCCCAACACCAACGGCTCCATCGGGCAC
TCTCCACTGTCTCTGTCTGCCCAGAGCGTGATGGAAGAACTGAAC
ACAGCCCCTGTGCAAGAGTCCCCTCCTCTGGCTATGCCTCCTGGC
AATTCTCACGGCCTGGAAGTGGGATCTCTGGCCGAAGTGAAAGAG
AACCCTCCTTTCTACGGCGTGATCCGGTGGATCGGACAACCTCCT
GGACTGAATGAAGTGCTGGCCGGACTGGAACTGGAAGATGAGTGT
GCCGGCTGCACCGACGGCACCTTTAGAGGCACCAGATACTTCACA
TGCGCCCTGAAGAAAGCCCTGTTCGTGAAGCTGAAGTCCTGCAGA
CCCGACAGCAGATTCGCTAGCCTGCAGCCTGTGTCCAATCAGATC
GAGCGGTGCAACTCCCTGGCCTTTGGCGGCTATCTGTCCGAGGTG
GTGGAAGAGAACACCCCTCCTAAGATGGAAAAAGAGGGCCTCGAG
ATTATGATCGGGAAGAAGAAGGGCATCCAGGGGCACTACAATAGC
TGCTACCTGGACAGCACCCTGTTCTGCCTGTTCGCCTTTAGCAGC
GTGCTGGACACTGTGCTGCTGCGGCCCAAAGAGAAGAACGACGTC
GAGTACTACAGCGAGACACAAGAGCTGCTGAGAACCGAGATCGTG
AACCCTCTGCGGATCTACGGCTACGTGTGCGCCACCAAGATCATG
AAGCTGCGGAAGATTCTGGAAAAGGTGGAAGCCGCCTCCGGCTTC
ACCAGCGAGGAAAAGGATCCCGAAGAGTTCCTGAACATCCTGTTT
CACCACATCCTGAGAGTGGAACCCCTGCTGAAGATCAGATCCGCC
GGACAGAAAGTGCAGGACTGCTACTTCTACCAGATCTTCATGGAA
AAGAACGAGAAAGTCGGCGTGCCCACCATCCAGCAACTGCTCGAG
TGGTCCTTCATCAACAGCAACCTGAAGTTCGCCGAGGCTCCCAGC
TGCCTGATCATCCAGATGCCTAGATTCGGCAAGGACTTCAAGCTG
TTCAAAAAGATCTTCCCCAGCCTCGAGCTGAACATCACCGACCTG
CTCGAGGACACCCCTCGGCAGTGTAGAATTTGTGGCGGCCTGGCT
ATGTACGAGTGCAGAGAGTGCTACGACGACCCCGATATCAGCGCC
GGCAAGATCAAGCAGTTCTGCAAGACCTGCAACACCCAAGTGCAT
CTGCACCCCAAGCGGCTGAACCACAAGTACAACCCCGTGTCTCTG
CCCAAGGACCTGCCTGACTGGGATTGGAGACACGGCTGTATCCCT
TGCCAGAACATGGAACTGTTCGCTGTGCTGTGCATCGAGACAAGC
CACTACGTGGCCTTCGTGAAGTACGGCAAGGATGACAGCGCCTGG
CTGTTCTTCGACAGCATGGCCGATAGAGATGGCGGCCAGAACGGC
TTCAACATCCCTCAAGTGACCCCTTGTCCTGAAGTGGGAGAGTAC
CTGAAGATGAGCCTGGAAGATCTGCACAGCCTGGACTCCAGACGG
ATCCAGGGATGTGCTAGAAGGCTGCTGTGCGACGCCTACATGTGC
ATGTATCAGAGCCCCACCATGAGCCTGTACAAGTGA

Hence, preferably the RNA construct is encoded by a DNA sequence substantially as set out in SEQ ID No: 179, or a fragment or variant thereof.

In an embodiment, the RNA sequence corresponding to the codon optimised DNA sequence of SEQ ID No: 179 that includes a start (AUG) and a stop (UGA) codon is provided herein as SEQ ID No: 180, as follows:

[SEQ ID No: 180]
AUGUCUAGCGGCCUGUGGUCCCAAGAGAAAGUGACAAGCCCCUAC
UGGGAAGAGAGGAUCUUCUACCUGCUGCUGCAAGAGUGCAGCGUG
ACCGACAAGCAGACCCAGAAACUGCUGAAGGUGCCCAAGGGCAGC
AUCGGCCAGUACAUCCAGGAUAGAAGCGUGGGCCACAGCAGAAUC
CCUAGCGCCAAGGGCAAGAAGAACCAGAUCGGCCUGAAGAUCCUG
GAACAGCCUCACGCCGUGCUGUUCGUGGACGAGAAGGACGUGGUG
GAAAUCAACGAGAAGUUCACCGAGCUGCUGCUGGCCAUCACCAAC
UGCGAGGAACGGUUCAGCCUGUUCAAGAACCGGAACCGGCUGAGC
AAGGGCCUGCAGAUCGAUGUGGGAUGCCCUGUGAAGGUGCAGCUG
AGAAGCGGCGAAGAGAAGUUCCCUGGCGUCGUGCGGUUUAGAGGA
CCUCUGCUGGCCGAGAGAACCGUGUCCGGCAUCUUCUUUGGCGUG
GAACUGCUGGAAGAAGGCAGAGGCCAGGGCUUUACCGAUGGCGUG
UACCAGGGCAAGCAGCUGUUUCAGUGCGACGAGGAUUGCGGCGUG
UUCGUGGCCCUGGAUAAGCUGGAACUGAUCGAGGACGACGACACA
GCCCUGGAAAGCGAUUAUGCCGGACCUGGCGAUACCAUGCAGGUC
GAACUGCCUCCACUCGAGAUCAACAGCCGGGUGUCCCUGAAAGUG
GGCGAGACAAUCGAGAGCGGCACCGUGAUCUUUUGCGACGUGCUG
CCUGGCAAAGAGUCCCUGGGCUAUUUUGUGGGCGUCGACAUGGAC
AACCCCAUCGGCAAUUGGGACGGCAGAUUUGACGGCGUGCAGCUG
UGCAGCUUCGCCUGUGUGGAAAGCACCAUCCUGCUGCACAUCAAC
GACAUCAUCCCCGCUCUGAGCGAGAGCGUGACCCAAGAAAGACGG
CCUCCUAAGCUGGCCUUCAUGUCUAGAGGCGUGGGCGAUAAGGGC
AGCUCCAGCCACAACAAGCCUAAGGCCACAGGCUCCACAAGCGAC
CCCGGCAACAGAAACAGAAGCGAGCUGUUCUACACCCUGAACGGC
AGCAGCGUGGACAGCCAGCCUCAGAGCAAGAGCAAGAACACCUGG
UACAUCGACGAGGUGGCCGAGGAUCCUGCCAAGAGCCUGACAGAG
AUCAGCACCGACUUCGACAGAAGCAGCCCUCCACUGCAGCCUCCA
CCUGUGAAUAGCCUGACCACCGAGAACAGAUUCCACAGCCUGCCU
UUCAGCCUGACUAAGAUGCCCAACACCAACGGCUCCAUCGGGCAC
UCUCCACUGUCUCUGUCUGCCCAGAGCGUGAUGGAAGAACUGAAC
ACAGCCCCUGUGCAAGAGUCCCCUCCUCUGGCUAUGCCUCCUGGC
AAUUCUCACGGCCUGGAAGUGGGAUCUCUGGCCGAAGUGAAAGAG
AACCCUCCUUUCUACGGCGUGAUCCGGUGGAUCGGACAACCUCCU
GGACUGAAUGAAGUGCUGGCCGGACUGGAACUGGAAGAUGAGUGU
GCCGGCUGCACCGACGGCACCUUUAGAGGCACCAGAUACUUCACA
UGCGCCCUGAAGAAAGCCCUGUUCGUGAAGCUGAAGUCCUGCAGA
CCCGACAGCAGAUUCGCUAGCCUGCAGCCUGUGUCCAAUCAGAUC
GAGCGGUGCAACUCCCUGGCCUUUGGCGGCUAUCUGUCCGAGGUG
GUGGAAGAGAACACCCCUCCUAAGAUGGAAAAAGAGGGCCUCGAG
AUUAUGAUCGGGAAGAAGAAGGGCAUCCAGGGGCACUACAAUAGC
UGCUACCUGGACAGCACCCUGUUCUGCCUGUUCGCCUUUAGCAGC
GUGCUGGACACUGUGCUGCUGCGGCCCAAAGAGAAGAACGACGUC
GAGUACUACAGCGAGACACAAGAGCUGCUGAGAACCGAGAUCGUG
AACCCUCUGCGGAUCUACGGCUACGUGUGCGCCACCAAGAUCAUG
AAGCUGCGGAAGAUUCUGGAAAAGGUGGAAGCCGCCUCCGGCUUC
ACCAGCGAGGAAAAGGAUCCCGAAGAGUUCCUGAACAUCCUGUUU
CACCACAUCCUGAGAGUGGAACCCCUGCUGAAGAUCAGAUCCGCC
GGACAGAAAGUGCAGGACUGCUACUUCUACCAGAUCUUCAUGGAA
AAGAACGAGAAAGUCGGCGUGCCCACCAUCCAGCAACUGCUCGAG
UGGUCCUUCAUCAACAGCAACCUGAAGUUCGCCGAGGCUCCCAGC
UGCCUGAUCAUCCAGAUGCCUAGAUUCGGCAAGGACUUCAAGCUG
UUCAAAAAGAUCUUCCCCAGCCUCGAGCUGAACAUCACCGACCUG
CUCGAGGACACCCCUCGGCAGUGUAGAAUUUGUGGCGGCCUGGCU
AUGUACGAGUGCAGAGAGUGCUACGACGACCCCGAUAUCAGCGCC
GGCAAGAUCAAGCAGUUCUGCAAGACCUGCAACACCCAAGUGCAU
CUGCACCCCAAGCGGCUGAACCACAAGUACAACCCCGUGUCUCUG
CCCAAGGACCUGCCUGACUGGGAUUGGAGACACGGCUGUAUCCCU
UGCCAGAACAUGGAACUGUUCGCUGUGCUGUGCAUCGAGACAAGC
CACUACGUGGCCUUCGUGAAGUACGGCAAGGAUGACAGCGCCUGG
CUGUUCUUCGACAGCAUGGCCGAUAGAGAUGGCGGCCAGAACGGC
UUCAACAUCCCUCAAGUGACCCCUUGUCCUGAAGUGGGAGAGUAC
CUGAAGAUGAGCCUGGAAGAUCUGCACAGCCUGGACUCCAGACGG
AUCCAGGGAUGUGCUAGAAGGCUGCUGUGCGACGCCUACAUGUGC
AUGUAUCAGAGCCCCACCAUGAGCCUGUACAAGUGA

Hence, preferably the RNA construct comprises a sequence substantially as set out in SEQ ID No: 180, or a fragment or variant thereof.

In one embodiment, the at least one IMP may be LGP2 (NCBI Reference Sequence: NM_024119.3; UniProtKB—Q96C10 (DHX58_HUMAN), or an orthologue thereof (Rothenfusser, S., N. Goutagny, G. DiPerna, M. Gong, B. G. Monks, A. Schoenemeyer, M. Yamamoto, S. Akira, K. A. Fitzgerald. 2005. The RNA helicase LGP2 inhibits TLR-independent sensing of viral replication by retinoic acid-inducible gene-I. J. Immunol. 175: 5260-5268; Komuro, A., C. M. Horvath. 2006. RNA and virus-independent inhibition of antiviral signaling by RNA helicase LGP2. J. Virol. 80: 12332-12342).

One embodiment of the LGP2 is represented herein as SEQ ID No: 181, as follows:

[SEQ ID No: 181]
MELRSYQWEVIMPALEGKNIIIWLPTGAGKTRAAAYVAKRHLETV
DGAKVVVLVNRVHLVTQHGEEFRRMLDGRWTVTTLSGDMGPRAGF
GHLARCHDLLICTAELLQMALTSPEEEEHVELTVFSLIVVDECHH
THKDTVYNVIMSQYLELKLQRAQPLPQVLGLTASPGTGGASKLDG
AINHVLQLCANLDTWCIMSPQNCCPQLQEHSQQPCKQYNLCHRRS
QDPFGDLLKKLMDQIHDHLEMPELSRKFGTQMYEQQVVKLSEAAA
LAGLQEQRVYALHLRRYNDALLIHDTVRAVDALAALQDFYHREHV
TKTQILCAERRLLALFDDRKNELAHLATHGPENPKLEMLEKILQR
QFSSSNSPRGIIFTRTRQSAHSLLLWLQQQQGLQTVDIRAQLLIG
AGNSSQSTHMTQRDQQEVIQKFQDGTLNLLVATSVAEEGLDIPHC
NVVVRYGLLTNEISMVQARGRARADQSVYAFVATEGSRELKRELI
NEALETLMEQAVAAVQKMDQAEYQAKIRDLQQAALTKRAAQAAQR
ENQRQQFPVEHVQLLCINCMVAVGHGSDLRKVEGTHHVNVNPNFS
NYYNVSRDPVVINKVFKDWKPGGVISCRNCGEVWGLQMIYKSVKL
PVLKVRSMLLETPQGRIQAKKWSRVPFSVPDFDFLQHCAENLSDL
SLD

Therefore, preferably the RNA construct of the first aspect comprises a nucleotide sequence which encodes an amino acid sequence substantially as set out in SEQ ID No: 181, or a variant or fragment thereof.

In one embodiment, the LGP2 polypeptide is encoded by the DNA nucleotide sequence of SEQ ID No: 182, as follows:

[SEQ ID No: 182]
ATGGAGCTTCGGTCCTACCAATGGGAGGTGATCATGCCTGCCCTG
GAGGGCAAGAATATCATCATCTGGCTGCCCACGGGTGCCGGGAAG
ACCCGGGCGGCTGCTTATGTGGCCAAGCGGCACCTAGAGACTGTG
GATGGAGCCAAGGTGGTTGTATTGGTCAACAGGGTGCACCTGGTG
ACCCAGCATGGTGAAGAGTTCAGGCGCATGCTGGATGGACGCTGG
ACCGTGACAACCCTGAGTGGGGACATGGGACCACGTGCTGGCTTT
GGCCACCTGGCCCGGTGCCATGACCTGCTCATCTGCACAGCAGAG
CTTCTGCAGATGGCACTGACCAGCCCCGAGGAGGAGGAGCACGTG
GAGCTCACTGTCTTCTCCCTGATCGTGGTGGATGAGTGCCACCAC
ACGCACAAGGACACCGTCTACAACGTCATCATGAGCCAGTACCTA
GAACTTAAACTCCAGAGGGCACAGCCGCTACCCCAGGTGCTGGGT
CTCACAGCCTCCCCAGGCACTGGCGGGGCCTCCAAACTCGATGGG
GCCATCAACCACGTCCTGCAGCTCTGTGCCAACTTGGACACGTGG
TGCATCATGTCACCCCAGAACTGCTGCCCCCAGCTGCAGGAGCAC
AGCCAACAGCCTTGCAAACAGTACAACCTCTGCCACAGGCGCAGC
CAGGATCCGTTTGGGGACTTGCTGAAGAAGCTCATGGACCAAATC
CATGACCACCTGGAGATGCCTGAGTTGAGCCGGAAATTTGGGACG
CAAATGTATGAGCAGCAGGTGGTGAAGCTGAGTGAGGCTGCGGCT
TTGGCTGGGCTTCAGGAGCAACGGGTGTATGCGCTTCACCTGAGG
CGCTACAATGACGCGCTGCTCATCCATGACACCGTCCGCGCCGTG
GATGCCTTGGCTGCGCTGCAGGATTTCTATCACAGGGAGCACGTC
ACTAAAACCCAGATCCTGTGTGCCGAGCGCCGGCTGCTGGCCCTG
TTCGATGACCGCAAGAATGAGCTGGCCCACTTGGCAACTCATGGC
CCAGAGAATCCAAAACTGGAGATGCTGGAAAAGATCCTGCAAAGG
CAGTTCAGTAGCTCTAACAGCCCTCGGGGTATCATCTTCACCCGC
ACCCGCCAAAGCGCACACTCCCTCCTGCTCTGGCTCCAGCAGCAG
CAGGGCCTGCAGACTGTGGACATCCGGGCCCAGCTACTGATTGGG
GCTGGGAACAGCAGCCAGAGCACCCACATGACCCAGAGGGACCAG
CAAGAAGTGATCCAGAAGTTCCAAGATGGAACCCTGAACCTTCTG
GTGGCCACGAGTGTGGCGGAGGAGGGGCTGGACATCCCACATTGC
AATGTGGTGGTGCGTTATGGGCTCTTGACCAATGAAATCTCCATG
GTCCAGGCCAGGGGCCGTGCCCGGGCCGATCAGAGTGTATACGCG
TTTGTAGCAACTGAAGGTAGCCGGGAGCTGAAGCGGGAGCTGATC
AACGAGGCGCTGGAGACGCTGATGGAGCAGGCAGTGGCTGCTGTG
CAGAAAATGGACCAGGCCGAGTACCAGGCCAAGATCCGGGATCTG
CAGCAGGCAGCCTTGACCAAGCGGGCGGCCCAGGCAGCCCAGCGG
GAGAACCAGCGGCAGCAGTTCCCAGTGGAGCACGTGCAGCTACTC
TGCATCAACTGCATGGTGGCTGTGGGCCATGGCAGCGACCTGCGG
AAGGTGGAGGGCACCCACCATGTCAATGTGAACCCCAACTTCTCG
AACTACTATAATGTCTCCAGGGATCCTGTGGTCATCAACAAAGTC
TTCAAGGACTGGAAGCCTGGGGGTGTCATCAGCTGCAGGAACTGT
GGGGAGGTCTGGGGTCTGCAGATGATCTACAAGTCAGTGAAGCTG
CCAGTGCTCAAAGTCCGCAGCATGCTGCTGGAGACCCCTCAGGGG
CGGATCCAGGCCAAAAAGTGGTCCCGCGTGCCCTTCTCCGTGCCT
GACTTTGACTTCCTGCAGCATTGTGCCGAGAACTTGTCGGACCTC
TCCCTGGAC

Accordingly, preferably the LGP2 polypeptide is encoded by the DNA nucleotide sequence substantially as set out in SEQ ID NO: 182, or a variant or fragment thereof.

Thus, the RNA construct may comprise an RNA nucleotide sequence of SEQ ID No: 183, as follows:

[SEQ ID No: 183]
AUGGAGCUUCGGUCCUACCAAUGGGAGGUGAUCAUGCCUGCCCUG
GAGGGCAAGAAUAUCAUCAUCUGGCUGCCCACGGGUGCCGGGAAG
ACCCGGGCGGCUGCUUAUGUGGCCAAGCGGCACCUAGAGACUGUG
GAUGGAGCCAAGGUGGUUGUAUUGGUCAACAGGGUGCACCUGGUG
ACCCAGCAUGGUGAAGAGUUCAGGCGCAUGCUGGAUGGACGCUGG
ACCGUGACAACCCUGAGUGGGGACAUGGGACCACGUGCUGGCUUU
GGCCACCUGGCCCGGUGCCAUGACCUGCUCAUCUGCACAGCAGAG
CUUCUGCAGAUGGCACUGACCAGCCCCGAGGAGGAGGAGCACGUG
GAGCUCACUGUCUUCUCCCUGAUCGUGGUGGAUGAGUGCCACCAC
ACGCACAAGGACACCGUCUACAACGUCAUCAUGAGCCAGUACCUA
GAACUUAAACUCCAGAGGGCACAGCCGCUACCCCAGGUGCUGGGU
CUCACAGCCUCCCCAGGCACUGGCGGGGCCUCCAAACUCGAUGGG
GCCAUCAACCACGUCCUGCAGCUCUGUGCCAACUUGGACACGUGG
UGCAUCAUGUCACCCCAGAACUGCUGCCCCCAGCUGCAGGAGCAC
AGCCAACAGCCUUGCAAACAGUACAACCUCUGCCACAGGCGCAGC
CAGGAUCCGUUUGGGGACUUGCUGAAGAAGCUCAUGGACCAAAUC
CAUGACCACCUGGAGAUGCCUGAGUUGAGCCGGAAAUUUGGGACG
CAAAUGUAUGAGCAGCAGGUGGUGAAGCUGAGUGAGGCUGCGGCU
UUGGCUGGGCUUCAGGAGCAACGGGUGUAUGCGCUUCACCUGAGG
CGCUACAAUGACGCGCUGCUCAUCCAUGACACCGUCCGCGCCGUG
GAUGCCUUGGCUGCGCUGCAGGAUUUCUAUCACAGGGAGCACGUC
ACUAAAACCCAGAUCCUGUGUGCCGAGCGCCGGCUGCUGGCCCUG
UUCGAUGACCGCAAGAAUGAGCUGGCCCACUUGGCAACUCAUGGC
CCAGAGAAUCCAAAACUGGAGAUGCUGGAAAAGAUCCUGCAAAGG
CAGUUCAGUAGCUCUAACAGCCCUCGGGGUAUCAUCUUCACCCGC
ACCCGCCAAAGCGCACACUCCCUCCUGCUCUGGCUCCAGCAGCAG
CAGGGCCUGCAGACUGUGGACAUCCGGGCCCAGCUACUGAUUGGG
GCUGGGAACAGCAGCCAGAGCACCCACAUGACCCAGAGGGACCAG
CAAGAAGUGAUCCAGAAGUUCCAAGAUGGAACCCUGAACCUUCUG
GUGGCCACGAGUGUGGCGGAGGAGGGGCUGGACAUCCCACAUUGC
AAUGUGGUGGUGCGUUAUGGGCUCUUGACCAAUGAAAUCUCCAUG
GUCCAGGCCAGGGGCCGUGCCCGGGCCGAUCAGAGUGUAUACGCG
UUUGUAGCAACUGAAGGUAGCCGGGAGCUGAAGCGGGAGCUGAUC
AACGAGGCGCUGGAGACGCUGAUGGAGCAGGCAGUGGCUGCUGUG
CAGAAAAUGGACCAGGCCGAGUACCAGGCCAAGAUCCGGGAUCUG
CAGCAGGCAGCCUUGACCAAGCGGGCGGCCCAGGCAGCCCAGCGG
GAGAACCAGCGGCAGCAGUUCCCAGUGGAGCACGUGCAGCUACUC
UGCAUCAACUGCAUGGUGGCUGUGGGCCAUGGCAGCGACCUGCGG
AAGGUGGAGGGCACCCACCAUGUCAAUGUGAACCCCAACUUCUCG
AACUACUAUAAUGUCUCCAGGGAUCCUGUGGUCAUCAACAAAGUC
UUCAAGGACUGGAAGCCUGGGGGUGUCAUCAGCUGCAGGAACUGU
GGGGAGGUCUGGGGUCUGCAGAUGAUCUACAAGUCAGUGAAGCUG
CCAGUGCUCAAAGUCCGCAGCAUGCUGCUGGAGACCCCUCAGGGG
CGGAUCCAGGCCAAAAAGUGGUCCCGCGUGCCCUUCUCCGUGCCU
GACUUUGACUUCCUGCAGCAUUGUGCCGAGAACUUGUCGGACCUC
UCCCUGGAC

Therefore, preferably the RNA construct comprises an RNA nucleotide sequence substantially as set out in SEQ ID No: 183, or a variant or fragment thereof.

The inventors then subjected the protein sequence of SEQ ID No: 181 to codon optimisation for human expression, and one embodiment of the codon optimised nucleic acid (DNA) sequence that includes a start (ATG) and a stop (TGA) codon is provided herein as SEQ ID No: 184, as follows:

[SEQ ID No: 184]
ATGGAACTGCGGAGCTACCAGTGGGAAGTGATCATGCCTG
CTCTGGAAGGCAAGAACATCATCATCTGGCTGCCCACCGG
CGCTGGCAAAACAAGAGCTGCTGCCTACGTGGCCAAGCGG
CACCTGGAAACAGTGGATGGCGCTAAGGTGGTGGTGCTGG
TCAACAGAGTGCACCTGGTTACCCAGCACGGCGAGGAATT
CAGAAGAATGCTGGACGGCCGGTGGACCGTGACAACACTG
TCTGGCGATATGGGCCCTAGAGCCGGCTTTGGACACCTGG
CCAGATGCCACGATCTGCTGATCTGTACAGCCGAACTGCT
GCAGATGGCCCTGACAAGCCCTGAGGAAGAGGAACACGTC
GAGCTGACCGTGTTCAGCCTGATCGTGGTGGACGAGTGCC
ACCACACACACAAGGACACCGTGTACAACGTGATCATGAG
CCAGTACCTGGAACTGAAGCTGCAGAGAGCCCAGCCTCTG
CCTCAAGTGCTGGGACTGACAGCCTCTCCTGGAACAGGCG
GAGCCTCTAAACTGGACGGCGCCATCAATCACGTGCTGCA
GCTGTGCGCCAACCTGGATACCTGGTGCATCATGTCCCCA
CAGAACTGCTGTCCCCAGCTGCAAGAGCACTCTCAGCAGC
CCTGCAAGCAGTACAACCTGTGCCACAGAAGATCTCAGGA
CCCCTTCGGCGACCTGCTGAAGAAACTGATGGACCAGATC
CACGACCACCTCGAGATGCCCGAGCTGAGCAGAAAGTTCG
GCACCCAGATGTACGAGCAGCAGGTTGTGAAGCTGAGCGA
AGCCGCTGCTCTGGCCGGACTGCAAGAACAGAGAGTGTAC
GCCCTGCACCTGAGGCGGTACAATGATGCCCTGCTGATCC
ACGATACCGTGCGCGCTGTTGATGCTCTGGCTGCTCTGCA
GGATTTCTACCACCGCGAGCACGTGACCAAGACACAGATC
CTGTGTGCCGAGAGAAGGCTGCTGGCCCTGTTCGACGACA
GAAAGAATGAGCTGGCCCACCTGGCTACACACGGCCCCGA
AAATCCCAAGCTGGAAATGCTGGAAAAGATCCTGCAGCGG
CAGTTCAGCAGCAGCAACAGCCCTAGAGGCATCATCTTCA
CCCGGACCAGACAGAGCGCCCACTCTCTGCTGCTGTGGCT
GCAGCAACAACAGGGACTGCAGACCGTGGACATTAGGGCC
CAGCTGCTGATCGGAGCCGGCAATAGCTCTCAGAGCACCC
ACATGACCCAGCGGGACCAGCAAGAAGTGATCCAGAAGTT
CCAGGACGGCACCCTGAATCTGCTGGTGGCCACATCTGTG
GCTGAGGAAGGCCTGGATATCCCTCACTGCAACGTGGTCG
TCAGATACGGCCTGCTGACCAACGAGATCAGCATGGTGCA
GGCCAGAGGCAGAGCCAGAGCCGATCAGTCTGTGTACGCC
TTCGTGGCTACAGAGGGCTCCAGAGAGCTGAAGCGCGAGC
TGATCAATGAGGCCCTGGAAACCCTGATGGAACAAGCCGT
GGCCGCCGTGCAGAAAATGGATCAGGCCGAGTACCAGGCC
AAGATCAGGGATCTGCAACAGGCCGCTCTGACCAAGAGAG
CTGCTCAGGCTGCCCAGAGAGAGAACCAGAGACAGCAATT
CCCCGTGGAACACGTGCAGCTGCTGTGTATCAACTGCATG
GTGGCCGTCGGACACGGCAGCGATCTGAGAAAAGTGGAAG
GCACCCACCACGTGAACGTGAACCCCAACTTCAGCAACTA
CTACAACGTGTCCAGAGATCCCGTGGTCATCAACAAGGTG
TTCAAGGACTGGAAGCCTGGCGGCGTGATCAGCTGCAGAA
ATTGCGGAGAAGTGTGGGGCCTGCAGATGATCTACAAGAG
CGTGAAGCTGCCCGTGCTGAAAGTGCGGAGCATGCTGCTG
GAAACACCCCAGGGAAGAATCCAGGCCAAAAAGTGGTCCA
GAGTGCCCTTCAGCGTGCCCGACTTCGATTTCCTGCAGCA
CTGCGCCGAGAACCTGAGCGATCTGTCCCTGGATTGA

Hence, preferably the RNA construct is encoded by a DNA sequence substantially as set out in SEQ ID No: 184, or a fragment or variant thereof.

In an embodiment, the RNA sequence corresponding to the codon optimised DNA sequence of SEQ ID No: 184 that includes a start (AUG) and a stop (UGA) codon is provided herein as SEQ ID No: 185, as follows:

[SEQ ID No: 185]
AUGGAACUGCGGAGCUACCAGUGGGAAGUGAUCAUGCCUG
CUCUGGAAGGCAAGAACAUCAUCAUCUGGCUGCCCACCGG
CGCUGGCAAAACAAGAGCUGCUGCCUACGUGGCCAAGCGG
CACCUGGAAACAGUGGAUGGCGCUAAGGUGGUGGUGCUGG
UCAACAGAGUGCACCUGGUUACCCAGCACGGCGAGGAAUU
CAGAAGAAUGCUGGACGGCCGGUGGACCGUGACAACACUG
UCUGGCGAUAUGGGCCCUAGAGCCGGCUUUGGACACCUGG
CCAGAUGCCACGAUCUGCUGAUCUGUACAGCCGAACUGCU
GCAGAUGGCCCUGACAAGCCCUGAGGAAGAGGAACACGUC
GAGCUGACCGUGUUCAGCCUGAUCGUGGUGGACGAGUGCC
ACCACACACACAAGGACACCGUGUACAACGUGAUCAUGAG
CCAGUACCUGGAACUGAAGCUGCAGAGAGCCCAGCCUCUG
CCUCAAGUGCUGGGACUGACAGCCUCUCCUGGAACAGGCG
GAGCCUCUAAACUGGACGGCGCCAUCAAUCACGUGCUGCA
GCUGUGCGCCAACCUGGAUACCUGGUGCAUCAUGUCCCCA
CAGAACUGCUGUCCCCAGCUGCAAGAGCACUCUCAGCAGC
CCUGCAAGCAGUACAACCUGUGCCACAGAAGAUCUCAGGA
CCCCUUCGGCGACCUGCUGAAGAAACUGAUGGACCAGAUC
CACGACCACCUCGAGAUGCCCGAGCUGAGCAGAAAGUUCG
GCACCCAGAUGUACGAGCAGCAGGUUGUGAAGCUGAGCGA
AGCCGCUGCUCUGGCCGGACUGCAAGAACAGAGAGUGUAC
GCCCUGCACCUGAGGCGGUACAAUGAUGCCCUGCUGAUCC
ACGAUACCGUGCGCGCUGUUGAUGCUCUGGCUGCUCUGCA
GGAUUUCUACCACCGCGAGCACGUGACCAAGACACAGAUC
CUGUGUGCCGAGAGAAGGCUGCUGGCCCUGUUCGACGACA
GAAAGAAUGAGCUGGCCCACCUGGCUACACACGGCCCCGA
AAAUCCCAAGCUGGAAAUGCUGGAAAAGAUCCUGCAGCGG
CAGUUCAGCAGCAGCAACAGCCCUAGAGGCAUCAUCUUCA
CCCGGACCAGACAGAGCGCCCACUCUCUGCUGCUGUGGCU
GCAGCAACAACAGGGACUGCAGACCGUGGACAUUAGGGCC
CAGCUGCUGAUCGGAGCCGGCAAUAGCUCUCAGAGCACCC
ACAUGACCCAGCGGGACCAGCAAGAAGUGAUCCAGAAGUU
CCAGGACGGCACCCUGAAUCUGCUGGUGGCCACAUCUGUG
GCUGAGGAAGGCCUGGAUAUCCCUCACUGCAACGUGGUCG
UCAGAUACGGCCUGCUGACCAACGAGAUCAGCAUGGUGCA
GGCCAGAGGCAGAGCCAGAGCCGAUCAGUCUGUGUACGCC
UUCGUGGCUACAGAGGGCUCCAGAGAGCUGAAGCGCGAGC
UGAUCAAUGAGGCCCUGGAAACCCUGAUGGAACAAGCCGU
GGCCGCCGUGCAGAAAAUGGAUCAGGCCGAGUACCAGGCC
AAGAUCAGGGAUCUGCAACAGGCCGCUCUGACCAAGAGAG
CUGCUCAGGCUGCCCAGAGAGAGAACCAGAGACAGCAAUU
CCCCGUGGAACACGUGCAGCUGCUGUGUAUCAACUGCAUG
GUGGCCGUCGGACACGGCAGCGAUCUGAGAAAAGUGGAAG
GCACCCACCACGUGAACGUGAACCCCAACUUCAGCAACUA
CUACAACGUGUCCAGAGAUCCCGUGGUCAUCAACAAGGUG
UUCAAGGACUGGAAGCCUGGCGGCGUGAUCAGCUGCAGAA
AUUGCGGAGAAGUGUGGGGCCUGCAGAUGAUCUACAAGAG
CGUGAAGCUGCCCGUGCUGAAAGUGCGGAGCAUGCUGCUG
GAAACACCCCAGGGAAGAAUCCAGGCCAAAAAGUGGUCCA
GAGUGCCCUUCAGCGUGCCCGACUUCGAUUUCCUGCAGCA
CUGCGCCGAGAACCUGAGCGAUCUGUCCCUGGAUUGA

Hence, preferably the RNA construct comprises a sequence substantially as set out in SEQ ID No: 185, or a fragment or variant thereof.

In one embodiment, the at least one IMP may be a DDX-56 (NCBI Reference Sequence: NM_019082.4; UniProtKB—Q9NY93 (DDX56_HUMAN), or an orthologue thereof (Li D, Fu S, Wu Z, Yang W, Ru Y, Shu H, Liu X, Zheng H. DDX56 inhibits type I interferon by disrupting assembly of IRF3-IPO5 to inhibit IRF3 nucleus import. J Cell Sci. 2020; 133(5): jcs230409). One embodiment of the DDX-56 is represented herein as SEQ ID No: 191, as follows:

[SEQ ID No: 191]
MEDSEALGFEHMGLDPRLLQAVTDLGWSRPTLIQEKAIPL
ALEGKDLLARARTGSGKTAAYAIPMLQLLLHRKATGPVVE
QAVRGLVLVPTKELARQAQSMIQQLATYCARDVRVANVSA
AEDSVSQRAVLMEKPDVVVGTPSRILSHLQQDSLKLRDSL
ELLVVDEADLLFSFGFEEELKSLLCHLPRIYQAFLMSATF
NEDVQALKELILHNPVTLKLQESQLPGPDQLQQFQVVCET
EEDKFLLLYALLKLSLIRGKSLLFVNTLERSYRLRLFLEQ
FSIPTCVLNGELPLRSRCHIISQFNQGFYDCVIATDAEVL
GAPVKGKRRGRGPKGDKASDPEAGVARGIDFHHVSAVLNF
DLPPTPEAYIHRAGRTARANNPGIVLTFVLPTEQFHLGKI
EELLSGENRGPILLPYQFRMEEIEGFRYRCRDAMRSVTKQ
AIREARLKEIKEELLHSEKLKTYFEDNPRDLQLLRHDLPL
HPAVVKPHLGHVPDYLVPPALRGLVRPHKKRKKLSSSCRK
AKRAKSQNPLRSFKHKGKKFRPTAKPS

Therefore, preferably the RNA construct of the first aspect comprises a nucleotide sequence which encodes an amino acid sequence substantially as set out in SEQ ID No: 191, or a variant or fragment thereof.

In one embodiment, the DDX-56 polypeptide is encoded by the DNA nucleotide sequence of SEQ ID No: 192, as follows:

[SEQ ID No: 192]
ATGGAGGACTCTGAAGCACTGGGCTTCGAACACATGGGCC
TCGATCCCCGGCTCCTTCAGGCTGTCACCGATCTGGGCTG
GTCGCGACCTACGCTGATCCAGGAGAAGGCCATCCCACTG
GCCCTAGAAGGGAAGGACCTCCTGGCTCGGGCCCGCACGG
GCTCCGGGAAGACGGCCGCTTATGCTATTCCGATGCTGCA
GCTGTTGCTCCATAGGAAGGCGACAGGTCCGGTGGTAGAA
CAGGCAGTGAGAGGCCTTGTTCTTGTTCCTACCAAGGAGC
TGGCACGGCAAGCACAGTCCATGATTCAGCAGCTGGCTAC
CTACTGTGCTCGGGATGTCCGAGTGGCCAATGTCTCAGCT
GCTGAAGACTCAGTCTCTCAGAGAGCTGTGCTGATGGAGA
AGCCAGATGTGGTAGTAGGGACCCCATCTCGCATATTAAG
CCACTTGCAGCAAGACAGCCTGAAACTTCGTGACTCCCTG
GAGCTTTTGGTGGTGGACGAAGCTGACCTTCTTTTTTCCT
TTGGCTTTGAAGAAGAGCTCAAGAGTCTCCTCTGTCACTT
GCCCCGGATTTACCAGGCTTTTCTCATGTCAGCTACTTTT
AACGAGGACGTACAAGCACTCAAGGAGCTGATATTACATA
ACCCGGTTACCCTTAAGTTACAGGAGTCCCAGCTGCCTGG
GCCAGACCAGTTACAGCAGTTTCAGGTGGTCTGTGAGACT
GAGGAAGACAAATTCCTCCTGCTGTATGCCCTGCTCAAGC
TGTCATTGATTCGGGGCAAGTCTCTGCTCTTTGTCAACAC
TCTAGAACGGAGTTACCGGCTACGCCTGTTCTTGGAACAG
TTCAGCATCCCCACCTGTGTGCTCAATGGAGAGCTTCCAC
TGCGCTCCAGGTGCCACATCATCTCACAGTTCAACCAAGG
CTTCTACGACTGTGTCATAGCAACTGATGCTGAAGTCCTG
GGGGCCCCAGTCAAGGGCAAGCGTCGGGGCCGAGGGCCCA
AAGGGGACAAGGCCTCTGATCCGGAAGCAGGTGTGGCCCG
GGGCATAGACTTCCACCATGTGTCTGCTGTGCTCAACTTT
GATCTTCCCCCAACCCCTGAGGCCTACATCCATCGAGCTG
GCAGGACAGCACGCGCTAACAACCCAGGCATAGTCTTAAC
CTTTGTGCTTCCCACGGAGCAGTTCCACTTAGGCAAGATT
GAGGAGCTTCTCAGTGGAGAGAACAGGGGCCCCATTCTGC
TCCCCTACCAGTTCCGGATGGAGGAGATCGAGGGCTTCCG
CTATCGCTGCAGGGATGCCATGCGCTCAGTGACTAAGCAG
GCCATTCGGGAGGCAAGATTGAAGGAGATCAAGGAAGAGC
TTCTGCATTCTGAGAAGCTTAAGACATACTTTGAAGACAA
CCCTAGGGACCTCCAGCTGCTGCGGCATGACCTACCTTTG
CACCCCGCAGTGGTGAAGCCCCACCTGGGCCATGTTCCTG
ACTACCTGGTTCCTCCTGCTCTCCGTGGCCTGGTGCGCCC
TCACAAGAAGCGGAAGAAGCTGTCTTCCTCTTGTAGGAAG
GCCAAGAGAGCAAAGTCCCAGAACCCACTGCGCAGCTTCA
AGCACAAAGGAAAGAAATTCAGACCCACAGCCAAGCCCTC
C

Accordingly, preferably the DDX-56 polypeptide is encoded by the DNA nucleotide sequence substantially as set out in SEQ ID NO: 192, or a variant or fragment thereof.

Thus, the RNA construct may comprise an RNA nucleotide sequence of SEQ ID No: 193, as follows:

[SEQ ID No: 193]
AUGGAGGACUCUGAAGCACUGGGCUUCGAACACAUGGGCC
UCGAUCCCCGGCUCCUUCAGGCUGUCACCGAUCUGGGCUG
GUCGCGACCUACGCUGAUCCAGGAGAAGGCCAUCCCACUG
GCCCUAGAAGGGAAGGACCUCCUGGCUCGGGCCCGCACGG
GCUCCGGGAAGACGGCCGCUUAUGCUAUUCCGAUGCUGCA
GCUGUUGCUCCAUAGGAAGGCGACAGGUCCGGUGGUAGAA
CAGGCAGUGAGAGGCCUUGUUCUUGUUCCUACCAAGGAGC
UGGCACGGCAAGCACAGUCCAUGAUUCAGCAGCUGGCUAC
CUACUGUGCUCGGGAUGUCCGAGUGGCCAAUGUCUCAGCU
GCUGAAGACUCAGUCUCUCAGAGAGCUGUGCUGAUGGAGA
AGCCAGAUGUGGUAGUAGGGACCCCAUCUCGCAUAUUAAG
CCACUUGCAGCAAGACAGCCUGAAACUUCGUGACUCCCUG
GAGCUUUUGGUGGUGGACGAAGCUGACCUUCUUUUUUCCU
UUGGCUUUGAAGAAGAGCUCAAGAGUCUCCUCUGUCACUU
GCCCCGGAUUUACCAGGCUUUUCUCAUGUCAGCUACUUUU
AACGAGGACGUACAAGCACUCAAGGAGCUGAUAUUACAUA
ACCCGGUUACCCUUAAGUUACAGGAGUCCCAGCUGCCUGG
GCCAGACCAGUUACAGCAGUUUCAGGUGGUCUGUGAGACU
GAGGAAGACAAAUUCCUCCUGCUGUAUGCCCUGCUCAAGC
UGUCAUUGAUUCGGGGCAAGUCUCUGCUCUUUGUCAACAC
UCUAGAACGGAGUUACCGGCUACGCCUGUUCUUGGAACAG
UUCAGCAUCCCCACCUGUGUGCUCAAUGGAGAGCUUCCAC
UGCGCUCCAGGUGCCACAUCAUCUCACAGUUCAACCAAGG
CUUCUACGACUGUGUCAUAGCAACUGAUGCUGAAGUCCUG
GGGGCCCCAGUCAAGGGCAAGCGUCGGGGCCGAGGGCCCA
AAGGGGACAAGGCCUCUGAUCCGGAAGCAGGUGUGGCCCG
GGGCAUAGACUUCCACCAUGUGUCUGCUGUGCUCAACUUU
GAUCUUCCCCCAACCCCUGAGGCCUACAUCCAUCGAGCUG
GCAGGACAGCACGCGCUAACAACCCAGGCAUAGUCUUAAC
CUUUGUGCUUCCCACGGAGCAGUUCCACUUAGGCAAGAUU
GAGGAGCUUCUCAGUGGAGAGAACAGGGGCCCCAUUCUGC
UCCCCUACCAGUUCCGGAUGGAGGAGAUCGAGGGCUUCCG
CUAUCGCUGCAGGGAUGCCAUGCGCUCAGUGACUAAGCAG
GCCAUUCGGGAGGCAAGAUUGAAGGAGAUCAAGGAAGAGC
UUCUGCAUUCUGAGAAGCUUAAGACAUACUUUGAAGACAA
CCCUAGGGACCUCCAGCUGCUGCGGCAUGACCUACCUUUG
CACCCCGCAGUGGUGAAGCCCCACCUGGGCCAUGUUCCUG
ACUACCUGGUUCCUCCUGCUCUCCGUGGCCUGGUGCGCCC
UCACAAGAAGCGGAAGAAGCUGUCUUCCUCUUGUAGGAAG
GCCAAGAGAGCAAAGUCCCAGAACCCACUGCGCAGCUUCA
AGCACAAAGGAAAGAAAUUCAGACCCACAGCCAAGCCCUC
C

Therefore, preferably the RNA construct comprises an RNA nucleotide sequence substantially as set out in SEQ ID No: 193, or a variant or fragment thereof.

The inventors then subjected the protein sequence of SEQ ID No: 191 to codon optimisation for human expression, and one embodiment of the codon optimised nucleic acid (DNA) sequence that includes a start (ATG) and a stop (TGA) codon is provided herein as SEQ ID No: 194, as follows:

[SEQ ID No: 194]
ATGGAAGATTCTGAGGCCCTGGGCTTCGAGCACATGGGCC
TTGATCCTAGACTGCTGCAGGCCGTGACAGATCTCGGATG
GTCCAGACCTACACTGATCCAAGAGAAGGCCATTCCTCTG
GCTCTGGAAGGCAAGGACCTGCTGGCCAGAGCTAGAACAG
GCTCTGGCAAGACAGCCGCCTACGCTATCCCTATGCTGCA
GCTGCTGCTGCACAGAAAGGCCACAGGACCAGTGGTGGAA
CAGGCCGTTAGAGGACTGGTGCTGGTGCCCACAAAAGAGC
TGGCTAGACAGGCCCAGAGCATGATCCAGCAGCTGGCCAC
ATACTGCGCCAGAGATGTGCGAGTGGCCAATGTGTCTGCC
GCCGAGGATTCTGTGTCTCAGAGGGCCGTGCTGATGGAAA
AGCCCGATGTGGTCGTGGGCACCCCTAGCAGAATCCTGTC
TCATCTGCAGCAGGACAGCCTGAAGCTGAGAGACAGCCTG
GAACTGCTGGTGGTGGATGAGGCCGATCTGCTGTTCAGCT
TCGGCTTCGAGGAAGAACTGAAGTCCCTGCTGTGCCATCT
GCCTCGGATCTACCAGGCCTTCCTGATGAGCGCCACCTTC
AACGAAGATGTGCAGGCCCTGAAAGAGCTGATCCTGCACA
ACCCCGTGACACTGAAGCTGCAAGAGAGCCAGCTGCCAGG
ACCTGATCAGCTCCAGCAGTTTCAAGTCGTGTGCGAGACA
GAAGAGGACAAGTTCCTGCTGCTGTACGCCCTGCTGAAGC
TGTCCCTGATCAGAGGCAAGAGCCTGCTGTTCGTGAACAC
CCTGGAAAGAAGCTACCGGCTGCGGCTGTTTCTGGAACAG
TTCAGCATCCCTACCTGCGTGCTGAACGGCGAGCTGCCTC
TGAGAAGCAGATGCCACATCATCAGCCAGTTCAACCAGGG
CTTCTACGACTGCGTGATCGCCACAGATGCCGAAGTGCTG
GGAGCACCCGTGAAGGGCAAAAGAAGAGGCAGAGGCCCCA
AGGGCGATAAGGCCAGTGATCCTGAAGCAGGCGTGGCCAG
AGGCATCGATTTTCACCATGTGTCCGCTGTGCTGAACTTC
GACCTGCCACCTACACCTGAGGCCTACATCCACAGAGCCG
GCAGAACAGCCAGAGCCAACAATCCTGGCATCGTGCTGAC
CTTCGTGCTGCCTACCGAACAGTTCCACCTGGGCAAGATC
GAAGAACTGCTGTCCGGCGAGAACAGGGGCCCTATCCTGC
TGCCTTACCAGTTCCGGATGGAAGAGATCGAGGGCTTCAG
ATACAGATGCAGGGACGCCATGCGGAGCGTGACAAAGCAG
GCCATTAGAGAGGCCCGGCTGAAAGAGATCAAAGAGGAAC
TGCTCCACAGCGAGAAGCTCAAGACCTACTTCGAGGACAA
CCCCAGGGACCTGCAGCTCCTGAGACATGATCTGCCTCTG
CACCCTGCCGTGGTCAAACCTCATCTGGGACACGTGCCCG
ACTACCTGGTTCCTCCTGCTCTGAGAGGCCTTGTGCGCCC
TCACAAGAAGCGGAAGAAGCTGAGCAGCTCTTGTCGGAAG
GCCAAGCGGGCCAAGAGCCAGAATCCACTGAGAAGCTTCA
AGCACAAGGGCAAGAAGTTCAGACCCACCGCCAAGCCTAG
CTGA

Hence, preferably the RNA construct is encoded by a DNA sequence substantially as set out in SEQ ID No: 194, or a fragment or variant thereof.

In an embodiment, the RNA sequence corresponding to the codon optimised DNA sequence of SEQ ID No: 194 that includes a start (AUG) and a stop (UGA) codon is provided herein as SEQ ID No: 195, as follows:

[SEQ ID No: 195]
AUGGAAGAUUCUGAGGCCCUGGGCUUCGAGCACAUGGGCC
UUGAUCCUAGACUGCUGCAGGCCGUGACAGAUCUCGGAUG
GUCCAGACCUACACUGAUCCAAGAGAAGGCCAUUCCUCUG
GCUCUGGAAGGCAAGGACCUGCUGGCCAGAGCUAGAACAG
GCUCUGGCAAGACAGCCGCCUACGCUAUCCCUAUGCUGCA
GCUGCUGCUGCACAGAAAGGCCACAGGACCAGUGGUGGAA
CAGGCCGUUAGAGGACUGGUGCUGGUGCCCACAAAAGAGC
UGGCUAGACAGGCCCAGAGCAUGAUCCAGCAGCUGGCCAC
AUACUGCGCCAGAGAUGUGCGAGUGGCCAAUGUGUCUGCC
GCCGAGGAUUCUGUGUCUCAGAGGGCCGUGCUGAUGGAAA
AGCCCGAUGUGGUCGUGGGCACCCCUAGCAGAAUCCUGUC
UCAUCUGCAGCAGGACAGCCUGAAGCUGAGAGACAGCCUG
GAACUGCUGGUGGUGGAUGAGGCCGAUCUGCUGUUCAGCU
UCGGCUUCGAGGAAGAACUGAAGUCCCUGCUGUGCCAUCU
GCCUCGGAUCUACCAGGCCUUCCUGAUGAGCGCCACCUUC
AACGAAGAUGUGCAGGCCCUGAAAGAGCUGAUCCUGCACA
ACCCCGUGACACUGAAGCUGCAAGAGAGCCAGCUGCCAGG
ACCUGAUCAGCUCCAGCAGUUUCAAGUCGUGUGCGAGACA
GAAGAGGACAAGUUCCUGCUGCUGUACGCCCUGCUGAAGC
UGUCCCUGAUCAGAGGCAAGAGCCUGCUGUUCGUGAACAC
CCUGGAAAGAAGCUACCGGCUGCGGCUGUUUCUGGAACAG
UUCAGCAUCCCUACCUGCGUGCUGAACGGCGAGCUGCCUC
UGAGAAGCAGAUGCCACAUCAUCAGCCAGUUCAACCAGGG
CUUCUACGACUGCGUGAUCGCCACAGAUGCCGAAGUGCUG
GGAGCACCCGUGAAGGGCAAAAGAAGAGGCAGAGGCCCCA
AGGGCGAUAAGGCCAGUGAUCCUGAAGCAGGCGUGGCCAG
AGGCAUCGAUUUUCACCAUGUGUCCGCUGUGCUGAACUUC
GACCUGCCACCUACACCUGAGGCCUACAUCCACAGAGCCG
GCAGAACAGCCAGAGCCAACAAUCCUGGCAUCGUGCUGAC
CUUCGUGCUGCCUACCGAACAGUUCCACCUGGGCAAGAUC
GAAGAACUGCUGUCCGGCGAGAACAGGGGCCCUAUCCUGC
UGCCUUACCAGUUCCGGAUGGAAGAGAUCGAGGGCUUCAG
AUACAGAUGCAGGGACGCCAUGCGGAGCGUGACAAAGCAG
GCCAUUAGAGAGGCCCGGCUGAAAGAGAUCAAAGAGGAAC
UGCUCCACAGCGAGAAGCUCAAGACCUACUUCGAGGACAA
CCCCAGGGACCUGCAGCUCCUGAGACAUGAUCUGCCUCUG
CACCCUGCCGUGGUCAAACCUCAUCUGGGACACGUGCCCG
ACUACCUGGUUCCUCCUGCUCUGAGAGGCCUUGUGCGCCC
UCACAAGAAGCGGAAGAAGCUGAGCAGCUCUUGUCGGAAG
GCCAAGCGGGCCAAGAGCCAGAAUCCACUGAGAAGCUUCA
AGCACAAGGGCAAGAAGUUCAGACCCACCGCCAAGCCUAG
CUGA

Hence, preferably the RNA construct comprises a sequence substantially as set out in SEQ ID No: 195, or a fragment or variant thereof.

In one embodiment, the at least one IMP may be ARL16 (NCBI Reference Sequence: NM_001040025.3; UniProtKB—Q0P5N6 (ARL16_HUMAN), or an orthologue thereof (Yang Y-K, Qu H, Gao D, Di W, Chen H-W, Guo X, He Z_H, Chen D-Y. ARF-like protein 16 (ARL16) inhibits RIG-I by binding with its C-terminal domain in a GTP-dependent manner. J Biol Chem 2011; 286(12):10568-10580). One embodiment of the ARL16 is represented herein as SEQ ID No: 196, as follows:

[SEQ ID No: 196]
MCLLLGATGVGKTLLVKRLQEVSSRDGKGDLGEPPPTRPT
VGTNLTDIVAQRKITIRELGGCMGPIWSSYYGNCRSLLFV
MDASDPTQLSASCVQLLGLLSAEQLAEASVLILFNKIDLP
CYMSTEEMKSLIRLPDIIACAKQNITTAEISAREGTGLAG
VLAWLQATHRAND

Therefore, preferably the RNA construct of the first aspect comprises a nucleotide sequence which encodes an amino acid sequence substantially as set out in SEQ ID No: 196, or a variant or fragment thereof.

In one embodiment, the ARL16 polypeptide is encoded by the DNA nucleotide sequence of SEQ ID No: 197, as follows:

[SEQ ID No: 197]
ATGTGTCTCCTGCTGGGGGCCACGGGCGTCGGGAAGACGC
TGCTGGTGAAACGGCTGCAGGAGGTGAGCTCCCGGGATGG
GAAAGGCGACCTGGGGGAGCCGCCCCCGACACGGCCCACG
GTGGGCACCAATCTTACTGACATCGTGGCACAGAGAAAGA
TCACCATCCGGGAGCTTGGGGGGTGCATGGGCCCCATCTG
GTCCAGTTACTATGGAAACTGCCGTTCTCTCCTGTTTGTG
ATGGACGCCTCTGACCCCACCCAGCTCTCTGCATCCTGTG
TGCAGCTCTTAGGTCTCCTTTCTGCAGAACAACTTGCAGA
AGCATCGGTGCTGATACTCTTCAATAAAATCGACCTACCC
TGTTACATGTCCACGGAGGAGATGAAGTCATTAATCAGGC
TTCCAGACATCATTGCTTGTGCCAAGCAGAACATCACCAC
GGCAGAAATCAGCGCCCGTGAAGGCACTGGCTTAGCAGGG
GTGCTGGCCTGGCTCCAGGCCACCCACAGAGCCAACGAT

Accordingly, preferably the ARL16 polypeptide is encoded by the DNA nucleotide sequence substantially as set out in SEQ ID NO: 197, or a variant or fragment thereof.

Thus, the RNA construct may comprise an RNA nucleotide sequence of SEQ ID No: 198, as follows:

[SEQ ID No: 198]
AUGUGUCUCCUGCUGGGGGCCACGGGCGUCGGGAAGACGC
UGCUGGUGAAACGGCUGCAGGAGGUGAGCUCCCGGGAUGG
GAAAGGCGACCUGGGGGAGCCGCCCCCGACACGGCCCACG
GUGGGCACCAAUCUUACUGACAUCGUGGCACAGAGAAAGA
UCACCAUCCGGGAGCUUGGGGGGUGCAUGGGCCCCAUCUG
GUCCAGUUACUAUGGAAACUGCCGUUCUCUCCUGUUUGUG
AUGGACGCCUCUGACCCCACCCAGCUCUCUGCAUCCUGUG
UGCAGCUCUUAGGUCUCCUUUCUGCAGAACAACUUGCAGA
AGCAUCGGUGCUGAUACUCUUCAAUAAAAUCGACCUACCC
UGUUACAUGUCCACGGAGGAGAUGAAGUCAUUAAUCAGGC
UUCCAGACAUCAUUGCUUGUGCCAAGCAGAACAUCACCAC
GGCAGAAAUCAGCGCCCGUGAAGGCACUGGCUUAGCAGGG
GUGCUGGCCUGGCUCCAGGCCACCCACAGAGCCAACGAU

Therefore, preferably the RNA construct comprises an RNA nucleotide sequence substantially as set out in SEQ ID No: 198, or a variant or fragment thereof.

The inventors then subjected the protein sequence of SEQ ID No: 196 to codon optimisation for human expression, and one embodiment of the codon optimised nucleic acid (DNA) sequence that includes a start (ATG) and a stop (TGA) codon is provided herein as SEQ ID No: 262, as follows:

[SEQ ID No: 262]
ATGTGTCTGCTGCTGGGAGCTACAGGCGTGGGCAAGACAC
TGCTGGTCAAGCGGCTGCAAGAGGTGTCCAGCAGAGATGG
CAAAGGCGATCTGGGAGAGCCTCCTCCAACCAGACCTACC
GTGGGCACCAACCTGACAGATATCGTGGCCCAGCGGAAGA
TCACCATCAGAGAACTCGGCGGCTGCATGGGCCCTATCTG
GTCTAGCTACTACGGCAACTGCCGCAGCCTGCTGTTCGTG
ATGGATGCCAGCGATCCCACACAGCTGAGCGCCTCTTGTG
TGCAACTGCTGGGACTGCTGTCTGCCGAACAACTGGCCGA
AGCCTCTGTGCTGATCCTGTTCAACAAGATCGACCTGCCT
TGCTACATGAGCACCGAGGAAATGAAGTCCCTGATCAGAC
TGCCCGACATCATTGCCTGCGCCAAGCAGAATATCACCAC
AGCCGAGATCAGCGCCAGAGAAGGCACAGGACTTGCTGGC
GTTCTGGCATGGCTGCAGGCCACACACAGAGCCAACGATT
GA

Hence, preferably the RNA construct is encoded by a DNA sequence substantially as set out in SEQ ID No: 262, or a fragment or variant thereof.

In an embodiment, the RNA sequence corresponding to the codon optimised DNA sequence of SEQ ID No: 262 that includes a start (AUG) and a stop (UGA) codon is provided herein as SEQ ID No: 263, as follows:

[SEQ ID No: 263]
AUGUGUCUGCUGCUGGGAGCUACAGGCGUGGGCAAGACAC
UGCUGGUCAAGCGGCUGCAAGAGGUGUCCAGCAGAGAUGG
CAAAGGCGAUCUGGGAGAGCCUCCUCCAACCAGACCUACC
GUGGGCACCAACCUGACAGAUAUCGUGGCCCAGCGGAAGA
UCACCAUCAGAGAACUCGGCGGCUGCAUGGGCCCUAUCUG
GUCUAGCUACUACGGCAACUGCCGCAGCCUGCUGUUCGUG
AUGGAUGCCAGCGAUCCCACACAGCUGAGCGCCUCUUGUG
UGCAACUGCUGGGACUGCUGUCUGCCGAACAACUGGCCGA
AGCCUCUGUGCUGAUCCUGUUCAACAAGAUCGACCUGCCU
UGCUACAUGAGCACCGAGGAAAUGAAGUCCCUGAUCAGAC
UGCCCGACAUCAUUGCCUGCGCCAAGCAGAAUAUCACCAC
AGCCGAGAUCAGCGCCAGAGAAGGCACAGGACUUGCUGGC
GUUCUGGCAUGGCUGCAGGCCACACACAGAGCCAACGAUU
GA

Hence, preferably the RNA construct comprises a sequence substantially as set out in SEQ ID No: 263, or a fragment or variant thereof.

In one embodiment, the at least one IMP may be ARL5B (NCBI Reference Sequence: NM_178815.5; UniProtKB—Q96KC2 (ARL5B_HUMAN), or an orthologue thereof. (Kitai Y, Takeuchi O, Kawasaki T, Ori D, Suevoshi T, Murase M, Akira S, Kawai T. Negative Regulation of Melanoma Differentiation-associated Gene 5 (MDA5)-dependent Antiviral Innate Immune Responses by Arf-like Protein 5B. J Bio Chem 2015; 290(2): 1269-1280). One embodiment of the ARL5B is represented herein as SEQ ID No: 199, as follows:

[SEQ ID No: 199]
MGLIFAKLWSLFCNQEHKVIIVGLDNAGKTTIYQFLMNEV
VHTSPTIGSNVEEIVVKNTHFLMWDIGGQESLRSSWNTYY
SNTEFIILVVDSIDRERLAITKEELYRMLAHEDLRKAAVL
IFANKQDMKGCMTAAEISKYLTLSSIKDHPWHIQSCCALT
GEGLCQGLEWMTSRIGVR

Therefore, preferably the RNA construct of the first aspect comprises a nucleotide sequence which encodes an amino acid sequence substantially as set out in SEQ ID No: 199, or a variant or fragment thereof.

In one embodiment, the ARL5B polypeptide is encoded by the DNA nucleotide sequence of SEQ ID No: 200, as follows:

[SEQ ID No: 200]
ATGGGGCTGATCTTCGCCAAACTGTGGAGCCTCTTCTGTA
ACCAAGAACACAAAGTAATTATAGTGGGACTGGATAATGC
AGGGAAAACCACCATTCTTTACCAATTCTTAATGAATGAA
GTGGTTCATACTTCTCCAACCATAGGAAGCAATGTTGAAG
AAATAGTTGTGAAGAACACTCATTTTCTTATGTGGGATAT
TGGTGGTCAGGAGTCTCTGCGATCATCCTGGAACACATAT
TACTCAAATACAGAGTTCATCATTCTTGTTGTTGATAGCA
TTGACAGGGAACGACTAGCTATTACAAAAGAAGAATTATA
CAGAATGTTGGCTCATGAGGATTTACGGAAGGCTGCAGTC
CTTATCTTTGCAAATAAACAGGATATGAAAGGGTGTATGA
CAGCAGCTGAAATCTCGAAATACCTCACCCTTAGTTCAAT
TAAGGATCATCCATGGCACATTCAATCCTGCTGTGCTCTC
ACAGGAGAAGGGTTATGCCAAGGTCTAGAGTGGATGACCT
CCCGGATTGGTGTGAGA

Accordingly, preferably the ARL5B polypeptide is encoded by the DNA nucleotide sequence substantially as set out in SEQ ID NO: 200, or a variant or fragment thereof.

Thus, the RNA construct may comprise an RNA nucleotide sequence of SEQ ID No: 201, as follows:

[SEQ ID No: 201]
AUGGGGCUGAUCUUCGCCAAACUGUGGAGCCUCUUCUGUA
ACCAAGAACACAAAGUAAUUAUAGUGGGACUGGAUAAUGC
AGGGAAAACCACCAUUCUUUACCAAUUCUUAAUGAAUGAA
GUGGUUCAUACUUCUCCAACCAUAGGAAGCAAUGUUGAAG
AAAUAGUUGUGAAGAACACUCAUUUUCUUAUGUGGGAUAU
UGGUGGUCAGGAGUCUCUGCGAUCAUCCUGGAACACAUAU
UACUCAAAUACAGAGUUCAUCAUUCUUGUUGUUGAUAGCA
UUGACAGGGAACGACUAGCUAUUACAAAAGAAGAAUUAUA
CAGAAUGUUGGCUCAUGAGGAUUUACGGAAGGCUGCAGUC
CUUAUCUUUGCAAAUAAACAGGAUAUGAAAGGGUGUAUGA
CAGCAGCUGAAAUCUCGAAAUACCUCACCCUUAGUUCAAU
UAAGGAUCAUCCAUGGCACAUUCAAUCCUGCUGUGCUCUC
ACAGGAGAAGGGUUAUGCCAAGGUCUAGAGUGGAUGACCU
CCCGGAUUGGUGUGAGA

Therefore, preferably the RNA construct comprises an RNA nucleotide sequence substantially as set out in SEQ ID No: 201, or a variant or fragment thereof.

The inventors then subjected the protein sequence of SEQ ID No: 199 to codon optimisation for human expression, and one embodiment of the codon optimised nucleic acid (DNA) sequence that includes that includes a start (ATG) and a stop (TGA) codon is provided herein as SEQ ID No: 202, as follows:

[SEQ ID No: 202]
ATGGGCCTGATCTTCGCCAAACTGTGGTCCCTGTTCTGCAATCAA
GAGCACAAAGTGATCATCGTCGGCCTGGACAACGCCGGCAAGACA
ACAATCCTGTACCAGTTCCTGATGAACGAGGTGGTGCACACAAGC
CCCACCATCGGCAGCAACGTGGAAGAGATCGTGGTCAAGAATACC
CACTTCCTGATGTGGGACATCGGCGGCCAAGAGAGCCTGAGAAGC
AGCTGGAACACCTACTACAGCAACACCGAGTTCATCATCCTGGTG
GTGGACAGCATCGACAGAGAGAGACTGGCCATCACCAAAGAGGAA
CTGTACCGGATGCTGGCCCACGAGGATCTGAGAAAAGCCGCCGTG
CTGATTTTTGCCAACAAGCAGGACATGAAGGGCTGCATGACAGCC
GCCGAGATCAGCAAGTACCTGACACTGAGCAGCATCAAGGATCAC
CCCTGGCACATCCAGAGCTGCTGTGCATTGACAGGCGAGGGCCTG
TGTCAGGGACTCGAGTGGATGACAAGCAGAATCGGCGTGCGGTGA

Hence, preferably the RNA construct is encoded by a DNA sequence substantially as set out in SEQ ID No: 202, or a fragment or variant thereof.

In an embodiment, the RNA sequence corresponding to the codon optimised DNA sequence of SEQ ID No: 202 that includes a start (AUG) and a stop (UGA) codon is provided herein as SEQ ID No: 203, as follows:

[SEQ ID No: 203]
AUGGGCCUGAUCUUCGCCAAACUGUGGUCCCUGUUCUGCAAUCAA
GAGCACAAAGUGAUCAUCGUCGGCCUGGACAACGCCGGCAAGACA
ACAAUCCUGUACCAGUUCCUGAUGAACGAGGUGGUGCACACAAGC
CCCACCAUCGGCAGCAACGUGGAAGAGAUCGUGGUCAAGAAUACC
CACUUCCUGAUGUGGGACAUCGGCGGCCAAGAGAGCCUGAGAAGC
AGCUGGAACACCUACUACAGCAACACCGAGUUCAUCAUCCUGGUG
GUGGACAGCAUCGACAGAGAGAGACUGGCCAUCACCAAAGAGGAA
CUGUACCGGAUGCUGGCCCACGAGGAUCUGAGAAAAGCCGCCGUG
CUGAUUUUUGCCAACAAGCAGGACAUGAAGGGCUGCAUGACAGCC
GCCGAGAUCAGCAAGUACCUGACACUGAGCAGCAUCAAGGAUCAC
CCCUGGCACAUCCAGAGCUGCUGUGCAUUGACAGGCGAGGGCCUG
UGUCAGGGACUCGAGUGGAUGACAAGCAGAAUCGGCGUGCGGUGA

Hence, preferably the RNA construct comprises a sequence substantially as set out in SEQ ID No: 203, or a fragment or variant thereof.

In yet another embodiment the IMP, may be a dominant negative acting form of MAVS (ΔCARD domain) (NCBI Reference Sequence: NM_020746.4; UniProtKB—Q7Z434 (MAVS_HUMAN) or an orthologue thereof. MAVS acts downstream of DHX33, DDX58/RIG-I and IFIH1/MDA5, which detect intracellular dsRNA produced during viral replication, to coordinate pathways leading to the activation of NF-kappa-B, IRF3 and IRF7, and to the subsequent induction of IFN (Seth R B, Sun L, Zhijian C-K, Chen K. Identification and Characterization of MAVS, a mitochondrial antiviral Signaling Protein that Activates NF-κB and IRF3. Cell, 122, 5, 9, 669-682). One embodiment of the protein sequence of dominant negative acting form of MAVS is represented herein as SEQ ID No: 247, as follows:

[SEQ ID No: 247]
GCELVDLADEVASVYQSYQPRTSDRPPDPLEPPSLPAERPGPPTP
AAAHSIPYNSCREKEPSYPMPVQETQAPESPGENSEQALQTLSPR
AIPRNPDGGPLESSSDLAALSPLTSSGHQEQDTELGSTHTAGATS
SLTPSRGPVSPSVSFQPLARSTPRASRLPGPTGSVVSTGTSFSSS
SPGLASAGAAEGKQGAESDQAEPIICSSGAEAPANSLPSKVPTTL
MPVNTVALKVPANPASVSTVPSKLPTSSKPPGAVPSNALINPAPS
KLPINSTRAGMVPSKVPTSMVLTKVSASTVPTDGSSRNEETPAAP
TPAGATGGSSAWLDSSSENRGLGSELSKPGVLASQVDSPFSGCFE
DLAISASTSLGMGPCHGPEENEYKSEGTFGIHVAENPSIQLLEGN
PGPPADPDGGPRPQADRKFQEREVPCHRPSPGALWLQVAVTGVLV
VTLLVVLYRRRLH

Therefore, preferably the RNA construct of the first aspect comprises a nucleotide sequence which encodes an amino acid sequence substantially as set out in SEQ ID No: 247, or a variant or fragment thereof.

In one embodiment, the dominant negative acting form of MAVS polypeptide is encoded by the DNA nucleotide sequence of SEQ ID No: 248, as follows:

[SEQ ID No: 248]
GGCTGTGAGCTAGTTGATCTCGCGGACGAAGTGGCCTCTGTCTAC
CAGAGCTACCAGCCTCGGACCTCGGACCGTCCCCCAGACCCACTG
GAGCCACCGTCACTTCCTGCTGAGAGGCCAGGGCCCCCCACACCT
GCTGCGGCCCACAGCATCCCCTACAACAGCTGCAGAGAGAAGGAG
CCAAGTTACCCCATGCCTGTCCAGGAGACCCAGGCGCCAGAGTCC
CCAGGAGAGAATTCAGAGCAAGCCCTGCAGACGCTCAGCCCCAGA
GCCATCCCAAGGAATCCAGATGGTGGCCCCCTGGAGTCCTCCTCT
GACCTGGCAGCCCTCAGCCCTCTGACCTCCAGCGGGCATCAGGAG
CAGGACACAGAACTGGGCAGTACCCACACAGCAGGTGCGACCTCC
AGCCTCACACCATCCCGTGGGCCTGTGTCTCCATCTGTCTCCTTC
CAGCCCCTGGCCCGTTCCACCCCCAGGGCAAGCCGCTTGCCTGGA
CCCACAGGGTCAGTTGTATCTACTGGCACCTCCTTCTCCTCCTCA
TCCCCTGGCTTGGCCTCTGCAGGGGCTGCAGAGGGTAAACAGGGT
GCAGAGAGTGACCAGGCCGAGCCTATCATCTGCTCCAGTGGGGCA
GAGGCACCTGCCAACTCTCTGCCCTCCAAAGTGCCTACCACCTTG
ATGCCTGTGAACACAGTGGCCCTGAAAGTGCCTGCCAACCCAGCA
TCTGTCAGCACAGTGCCCTCCAAGTTGCCAACTAGCTCAAAGCCC
CCTGGTGCAGTGCCTTCTAATGCGCTCACCAATCCAGCACCATCC
AAATTGCCCATCAACTCAACCCGTGCTGGCATGGTGCCATCCAAA
GTGCCTACTAGCATGGTGCTCACCAAGGTGTCTGCCAGCACAGTC
CCCACTGACGGGAGCAGCAGAAATGAGGAGACCCCAGCAGCTCCA
ACACCCGCCGGCGCCACTGGAGGCAGCTCAGCCTGGCTAGACAGC
AGCTCTGAGAATAGGGGCCTTGGGTCGGAGCTGAGTAAGCCTGGC
GTGCTGGCATCCCAGGTAGACAGCCCGTTCTCGGGCTGCTTCGAG
GATCTTGCCATCAGTGCCAGCACCTCCTTGGGCATGGGGCCCTGC
CATGGCCCAGAGGAGAATGAGTATAAGTCCGAGGGCACCTTTGGG
ATCCACGTGGCTGAGAACCCCAGCATCCAGCTCCTGGAGGGCAAC
CCTGGGCCACCTGCGGACCCGGATGGCGGCCCCAGGCCACAAGCC
GACCGGAAGTTCCAGGAGAGGGAGGTGCCATGCCACAGGCCCTCA
CCTGGGGCTCTGTGGCTCCAGGTGGCTGTGACAGGGGTGCTGGTA
GTCACACTCCTGGTGGTGCTGTACCGGCGGCGTCTGCAC

Accordingly, preferably the dominant negative acting form of MAVS polypeptide is encoded by the DNA nucleotide sequence substantially as set out in SEQ ID NO: 248, or a variant or fragment thereof.

Thus, the RNA construct may comprise an RNA nucleotide sequence of SEQ ID No: 249, as follows:

[SEQ ID No: 249]
GGCUGUGAGCUAGUUGAUCUCGCGGACGAAGUGGCCUCUGUCUAC
CAGAGCUACCAGCCUCGGACCUCGGACCGUCCCCCAGACCCACUG
GAGCCACCGUCACUUCCUGCUGAGAGGCCAGGGCCCCCCACACCU
GCUGCGGCCCACAGCAUCCCCUACAACAGCUGCAGAGAGAAGGAG
CCAAGUUACCCCAUGCCUGUCCAGGAGACCCAGGCGCCAGAGUCC
CCAGGAGAGAAUUCAGAGCAAGCCCUGCAGACGCUCAGCCCCAGA
GCCAUCCCAAGGAAUCCAGAUGGUGGCCCCCUGGAGUCCUCCUCU
GACCUGGCAGCCCUCAGCCCUCUGACCUCCAGCGGGCAUCAGGAG
CAGGACACAGAACUGGGCAGUACCCACACAGCAGGUGCGACCUCC
AGCCUCACACCAUCCCGUGGGCCUGUGUCUCCAUCUGUCUCCUUC
CAGCCCCUGGCCCGUUCCACCCCCAGGGCAAGCCGCUUGCCUGGA
CCCACAGGGUCAGUUGUAUCUACUGGCACCUCCUUCUCCUCCUCA
UCCCCUGGCUUGGCCUCUGCAGGGGCUGCAGAGGGUAAACAGGGU
GCAGAGAGUGACCAGGCCGAGCCUAUCAUCUGCUCCAGUGGGGCA
GAGGCACCUGCCAACUCUCUGCCCUCCAAAGUGCCUACCACCUUG
AUGCCUGUGAACACAGUGGCCCUGAAAGUGCCUGCCAACCCAGCA
UCUGUCAGCACAGUGCCCUCCAAGUUGCCAACUAGCUCAAAGCCC
CCUGGUGCAGUGCCUUCUAAUGCGCUCACCAAUCCAGCACCAUCC
AAAUUGCCCAUCAACUCAACCCGUGCUGGCAUGGUGCCAUCCAAA
GUGCCUACUAGCAUGGUGCUCACCAAGGUGUCUGCCAGCACAGUC
CCCACUGACGGGAGCAGCAGAAAUGAGGAGACCCCAGCAGCUCCA
ACACCCGCCGGCGCCACUGGAGGCAGCUCAGCCUGGCUAGACAGC
AGCUCUGAGAAUAGGGGCCUUGGGUCGGAGCUGAGUAAGCCUGGC
GUGCUGGCAUCCCAGGUAGACAGCCCGUUCUCGGGCUGCUUCGAG
GAUCUUGCCAUCAGUGCCAGCACCUCCUUGGGCAUGGGGCCCUGC
CAUGGCCCAGAGGAGAAUGAGUAUAAGUCCGAGGGCACCUUUGGG
AUCCACGUGGCUGAGAACCCCAGCAUCCAGCUCCUGGAGGGCAAC
CCUGGGCCACCUGCGGACCCGGAUGGCGGCCCCAGGCCACAAGCC
GACCGGAAGUUCCAGGAGAGGGAGGUGCCAUGCCACAGGCCCUCA
CCUGGGGCUCUGUGGCUCCAGGUGGCUGUGACAGGGGUGCUGGUA
GUCACACUCCUGGUGGUGCUGUACCGGCGGCGUCUGCAC

Therefore, preferably the RNA construct comprises an RNA nucleotide sequence substantially as set out in SEQ ID No: 249, or a variant or fragment thereof.

The inventors then subjected the protein sequence of SEQ ID No: 247 to codon optimisation for human expression, and one embodiment of the codon optimised nucleic acid (DNA) sequence that includes a start (ATG) and a stop (TGA) codon is provided herein as SEQ ID No: 250, as follows:

[SEQ ID No: 250]
ATGGGCTGTGAACTGGTGGATCTGGCCGATGAAGTGGCCAGCGTG
TACCAGAGCTACCAGCCTAGAACCAGCGACCGGCCTCCTGATCCT
CTGGAACCTCCATCTCTGCCCGCCGAAAGACCTGGACCTCCTACA
CCAGCTGCCGCTCACAGCATCCCTTACAACAGCTGCAGAGAGAAA
GAACCTAGCTACCCCATGCCTGTGCAAGAGACACAGGCCCCAGAA
AGCCCTGGCGAGAATTCTGAACAGGCCCTGCAGACACTGAGCCCC
AGAGCCATTCCTAGAAACCCTGATGGCGGCCCTCTGGAAAGCAGC
AGTGATCTGGCTGCTCTGAGCCCTCTGACAAGCTCTGGACACCAA
GAGCAGGATACCGAGCTGGGCAGCACACATACAGCCGGCGCTACA
AGCAGCCTGACACCTTCTAGAGGCCCCGTGTCTCCCAGCGTGTCA
TTTCAGCCTCTGGCCAGGTCTACCCCTAGAGCCTCTAGACTGCCT
GGACCTACAGGCAGCGTGGTGTCTACCGGCACAAGCTTCAGCTCT
AGCTCTCCTGGACTGGCCTCTGCTGGTGCCGCTGAGGGAAAACAA
GGCGCCGAATCTGATCAGGCCGAGCCTATCATCTGTAGCAGCGGA
GCAGAAGCCCCTGCCAATAGCCTGCCTAGCAAGGTGCCAACCACA
CTGATGCCCGTGAACACAGTGGCCCTGAAGGTGCCAGCTAATCCT
GCCTCCGTGTCCACCGTGCCTTCTAAGCTGCCAACCAGCTCTAAG
CCACCTGGCGCCGTGCCATCTAACGCCCTGACAAATCCTGCTCCA
AGCAAGCTGCCCATCAACAGCACAAGAGCCGGCATGGTGCCCTCT
AAGGTGCCCACATCTATGGTGCTGACCAAGGTGTCCGCCAGCACC
GTGCCAACAGATGGCAGCAGCAGAAACGAGGAAACCCCTGCCGCT
CCTACTCCTGCTGGCGCTACAGGCGGATCTTCTGCCTGGCTGGAT
AGCAGCTCCGAGAATAGAGGCCTGGGCAGCGAGCTGTCTAAACCT
GGCGTTCTGGCAAGCCAGGTGGACAGCCCTTTCAGCGGCTGCTTT
GAGGACCTGGCTATCAGCGCCTCTACAAGCCTCGGCATGGGACCT
TGTCACGGCCCCGAGGAAAACGAGTACAAGAGCGAGGGCACCTTC
GGCATCCACGTGGCCGAGAATCCTAGCATCCAACTGCTGGAAGGC
AACCCCGGACCTCCTGCTGATCCAGATGGTGGACCTAGACCTCAG
GCCGACCGGAAGTTCCAAGAAAGAGAGGTGCCCTGCCACCGGCCA
TCTCCAGGTGCACTTTGGCTGCAAGTGGCTGTGACAGGCGTGCTG
GTGGTTACACTGCTGGTCGTGCTGTACAGAAGGCGGCTGCATTGA

Hence, preferably the RNA construct is encoded by a DNA sequence substantially as set out in SEQ ID No: 250, or a fragment or variant thereof.

In an embodiment, the RNA sequence corresponding to the codon optimised DNA sequence of SEQ ID No: 250 that includes a start (AUG) and a stop (UGA) codon is provided herein as SEQ ID No: 251, as follows:

[SEQ ID No: 251]
AUGGGCUGUGAACUGGUGGAUCUGGCCGAUGAAGUGGCCAGCGUG
UACCAGAGCUACCAGCCUAGAACCAGCGACCGGCCUCCUGAUCCU
CUGGAACCUCCAUCUCUGCCCGCCGAAAGACCUGGACCUCCUACA
CCAGCUGCCGCUCACAGCAUCCCUUACAACAGCUGCAGAGAGAAA
GAACCUAGCUACCCCAUGCCUGUGCAAGAGACACAGGCCCCAGAA
AGCCCUGGCGAGAAUUCUGAACAGGCCCUGCAGACACUGAGCCCC
AGAGCCAUUCCUAGAAACCCUGAUGGCGGCCCUCUGGAAAGCAGC
AGUGAUCUGGCUGCUCUGAGCCCUCUGACAAGCUCUGGACACCAA
GAGCAGGAUACCGAGCUGGGCAGCACACAUACAGCCGGCGCUACA
AGCAGCCUGACACCUUCUAGAGGCCCCGUGUCUCCCAGCGUGUCA
UUUCAGCCUCUGGCCAGGUCUACCCCUAGAGCCUCUAGACUGCCU
GGACCUACAGGCAGCGUGGUGUCUACCGGCACAAGCUUCAGCUCU
AGCUCUCCUGGACUGGCCUCUGCUGGUGCCGCUGAGGGAAAACAA
GGCGCCGAAUCUGAUCAGGCCGAGCCUAUCAUCUGUAGCAGCGGA
GCAGAAGCCCCUGCCAAUAGCCUGCCUAGCAAGGUGCCAACCACA
CUGAUGCCCGUGAACACAGUGGCCCUGAAGGUGCCAGCUAAUCCU
GCCUCCGUGUCCACCGUGCCUUCUAAGCUGCCAACCAGCUCUAAG
CCACCUGGCGCCGUGCCAUCUAACGCCCUGACAAAUCCUGCUCCA
AGCAAGCUGCCCAUCAACAGCACAAGAGCCGGCAUGGUGCCCUCU
AAGGUGCCCACAUCUAUGGUGCUGACCAAGGUGUCCGCCAGCACC
GUGCCAACAGAUGGCAGCAGCAGAAACGAGGAAACCCCUGCCGCU
CCUACUCCUGCUGGCGCUACAGGCGGAUCUUCUGCCUGGCUGGAU
AGCAGCUCCGAGAAUAGAGGCCUGGGCAGCGAGCUGUCUAAACCU
GGCGUUCUGGCAAGCCAGGUGGACAGCCCUUUCAGCGGCUGCUUU
GAGGACCUGGCUAUCAGCGCCUCUACAAGCCUCGGCAUGGGACCU
UGUCACGGCCCCGAGGAAAACGAGUACAAGAGCGAGGGCACCUUC
GGCAUCCACGUGGCCGAGAAUCCUAGCAUCCAACUGCUGGAAGGC
AACCCCGGACCUCCUGCUGAUCCAGAUGGUGGACCUAGACCUCAG
GCCGACCGGAAGUUCCAAGAAAGAGAGGUGCCCUGCCACCGGCCA
UCUCCAGGUGCACUUUGGCUGCAAGUGGCUGUGACAGGCGUGCUG
GUGGUUACACUGCUGGUCGUGCUGUACAGAAGGCGGCUGCAUUGA

Hence, preferably the RNA construct comprises a sequence substantially as set out in SEQ ID No: 251, or a fragment or variant thereof.

In another embodiment the IMP is TRIM35 or an orthologue thereof (NCBI Reference Sequence: NM_171982.4; UniProtKB—Q9UPQ4 (TRI35_HUMAN)).

TRIM35 has been shown to interact with IRF7 to induce its degradation via the K48-linked ubiquitin-proteasome pathway. (Wang Y, Yan S, Yang B, Wang Y, Zhou H, Lian Q, Sun B (2015). TRIM35 negatively regulates TLR7- and TLR9-mediated type 1 interferon production by targeting IRF7. FEBS Lett, 589, 12, 1322-1330). One embodiment of the protein sequence of TRIM35 is represented herein as SEQ ID No: 252, as follows:

[SEQ ID No: 252]
MERSPDVSPGPSRSFKEELLCAVCYDPFRDAVTLRCGHNFCRGCV
SRCWEVQVSPTCPVCKDRASPADLRINHTLNNLVEKLLREEAEGA
RWTSYRFSRVCRLHRGQLSLFCLEDKELLCCSCQADPRHQGHRVQ
PVKDTAHDFRAKCRNMEHALREKAKAFWAMRRSYEAIAKHNQVEA
AWLEGRIRQEFDKLREFLRVEEQAILDAMAEETRQKQLLADEKMK
QLTEETEVLAHEIERLQMEMKEDDVSFLMKHKSRKRRLFCTMEPE
PVQPGMLIDVCKYLGSLQYRVWKKMLASVESVPFSFDPNTAAGWL
SVSDDLTSVTNHGYRVQVENPERFSSAPCLLGSRVFSQGSHAWEV
ALGGLQSWRVGVVRVRQDSGAEGHSHSCYHDTRSGFWYVCRTQGV
EGDHCVTSDPATSPLVLAIPRRLRVELECEEGELSFYDAERHCHL
YTFHARFGEVRPYFYLGGARGAGPPEPLRICPLHISVKEELDG

Therefore, preferably the RNA construct of the first aspect comprises a nucleotide sequence which encodes an amino acid sequence substantially as set out in SEQ ID No: 252, or a variant or fragment thereof.

In one embodiment, the TRIM35 polypeptide is encoded by the DNA nucleotide sequence of SEQ ID No: 253, as follows:

[SEQ ID No: 253]
ATGGAGCGGAGTCCCGACGTGTCCCCCGGGCCTTCCCGCTCCTTC
AAGGAGGAGTTGCTCTGCGCCGTCTGCTACGACCCCTTCCGCGAC
GCAGTCACTCTGCGCTGCGGCCACAACTTCTGCCGCGGGTGCGTG
AGCCGCTGCTGGGAGGTGCAGGTGTCGCCCACCTGCCCAGTGTGC
AAAGACCGCGCGTCACCCGCCGACCTGCGCACCAACCACACCCTC
AACAACCTGGTGGAGAAGCTGCTGCGCGAGGAGGCCGAGGGCGCG
CGCTGGACCAGCTACCGCTTCTCGCGTGTCTGCCGCCTGCACCGC
GGACAGCTCAGCCTCTTCTGCCTCGAGGACAAGGAGCTGCTGTGC
TGCTCCTGCCAGGCCGACCCCCGACACCAGGGGCACCGCGTGCAG
CCGGTGAAGGACACTGCCCACGACTTTCGGGCCAAGTGCAGGAAC
ATGGAGCATGCACTGCGGGAGAAGGCCAAGGCCTTCTGGGCCATG
CGGCGCTCCTATGAGGCCATCGCCAAGCACAATCAGGTGGAGGCT
GCATGGCTGGAAGGCCGGATCCGGCAGGAGTTTGATAAGCTTCGC
GAGTTCTTGAGAGTGGAGGAGCAGGCCATTCTGGATGCCATGGCC
GAGGAGACAAGGCAGAAGCAACTTCTGGCCGACGAGAAGATGAAG
CAGCTCACAGAGGAGACGGAGGTGCTGGCACATGAGATCGAGCGG
CTGCAGATGGAGATGAAGGAGGACGACGTTTCTTTTCTCATGAAA
CACAAGAGCCGAAAACGCCGACTCTTCTGCACCATGGAGCCAGAG
CCAGTCCAGCCCGGCATGCTTATCGATGTCTGCAAGTACCTGGGC
TCCCTGCAGTACCGCGTCTGGAAGAAGATGCTTGCATCTGTGGAA
TCTGTACCCTTCAGCTTTGACCCCAACACCGCAGCTGGCTGGCTC
TCCGTGTCTGACGACCTCACCAGCGTCACCAACCATGGCTACCGC
GTGCAGGTGGAGAACCCGGAACGCTTCTCCTCGGCGCCCTGCCTG
CTGGGCTCCCGTGTCTTCTCACAGGGCTCGCACGCCTGGGAGGTG
GCCCTTGGGGGGCTGCAGAGCTGGAGGGTGGGCGTGGTACGTGTG
CGCCAGGACTCGGGCGCTGAGGGCCACTCACACAGCTGCTACCAC
GACACACGCTCGGGCTTCTGGTATGTCTGCCGCACGCAGGGCGTG
GAGGGGGACCACTGCGTGACCTCGGACCCAGCCACGTCGCCCCTG
GTCCTGGCCATCCCACGCCGCCTGCGTGTGGAGCTGGAGTGTGAG
GAGGGCGAGCTGTCTTTCTATGACGCGGAGCGCCACTGCCACCTG
TACACCTTCCACGCCCGCTTTGGGGAGGTTCGCCCCTACTTCTAC
CTGGGGGGTGCACGGGGCGCCGGGCCTCCAGAGCCTTTGCGCATC
TGCCCCTTGCACATCAGTGTCAAGGAAGAACTGGATGGC

Accordingly, preferably the TRIM35 polypeptide is encoded by the DNA nucleotide sequence substantially as set out in SEQ ID NO: 253, or a variant or fragment thereof.

Thus, the RNA construct may comprise an RNA nucleotide sequence of SEQ ID No: 254, as follows:

[SEQ ID No: 254]
AUGGAGCGGAGUCCCGACGUGUCCCCCGGGCCUUCCCGCUCCUUC
AAGGAGGAGUUGCUCUGCGCCGUCUGCUACGACCCCUUCCGCGAC
GCAGUCACUCUGCGCUGCGGCCACAACUUCUGCCGCGGGUGCGUG
AGCCGCUGCUGGGAGGUGCAGGUGUCGCCCACCUGCCCAGUGUGC
AAAGACCGCGCGUCACCCGCCGACCUGCGCACCAACCACACCCUC
AACAACCUGGUGGAGAAGCUGCUGCGCGAGGAGGCCGAGGGCGCG
CGCUGGACCAGCUACCGCUUCUCGCGUGUCUGCCGCCUGCACCGC
GGACAGCUCAGCCUCUUCUGCCUCGAGGACAAGGAGCUGCUGUGC
UGCUCCUGCCAGGCCGACCCCCGACACCAGGGGCACCGCGUGCAG
CCGGUGAAGGACACUGCCCACGACUUUCGGGCCAAGUGCAGGAAC
AUGGAGCAUGCACUGCGGGAGAAGGCCAAGGCCUUCUGGGCCAUG
CGGCGCUCCUAUGAGGCCAUCGCCAAGCACAAUCAGGUGGAGGCU
GCAUGGCUGGAAGGCCGGAUCCGGCAGGAGUUUGAUAAGCUUCGC
GAGUUCUUGAGAGUGGAGGAGCAGGCCAUUCUGGAUGCCAUGGCC
GAGGAGACAAGGCAGAAGCAACUUCUGGCCGACGAGAAGAUGAAG
CAGCUCACAGAGGAGACGGAGGUGCUGGCACAUGAGAUCGAGCGG
CUGCAGAUGGAGAUGAAGGAGGACGACGUUUCUUUUCUCAUGAAA
CACAAGAGCCGAAAACGCCGACUCUUCUGCACCAUGGAGCCAGAG
CCAGUCCAGCCCGGCAUGCUUAUCGAUGUCUGCAAGUACCUGGGC
UCCCUGCAGUACCGCGUCUGGAAGAAGAUGCUUGCAUCUGUGGAA
UCUGUACCCUUCAGCUUUGACCCCAACACCGCAGCUGGCUGGCUC
UCCGUGUCUGACGACCUCACCAGCGUCACCAACCAUGGCUACCGC
GUGCAGGUGGAGAACCCGGAACGCUUCUCCUCGGCGCCCUGCCUG
CUGGGCUCCCGUGUCUUCUCACAGGGCUCGCACGCCUGGGAGGUG
GCCCUUGGGGGGCUGCAGAGCUGGAGGGUGGGCGUGGUACGUGUG
CGCCAGGACUCGGGCGCUGAGGGCCACUCACACAGCUGCUACCAC
GACACACGCUCGGGCUUCUGGUAUGUCUGCCGCACGCAGGGCGUG
GAGGGGGACCACUGCGUGACCUCGGACCCAGCCACGUCGCCCCUG
GUCCUGGCCAUCCCACGCCGCCUGCGUGUGGAGCUGGAGUGUGAG
GAGGGCGAGCUGUCUUUCUAUGACGCGGAGCGCCACUGCCACCUG
UACACCUUCCACGCCCGCUUUGGGGAGGUUCGCCCCUACUUCUAC
CUGGGGGGUGCACGGGGCGCCGGGCCUCCAGAGCCUUUGCGCAUC
UGCCCCUUGCACAUCAGUGUCAAGGAAGAACUGGAUGGC

Therefore, preferably the RNA construct comprises an RNA nucleotide sequence substantially as set out in SEQ ID No: 254, or a variant or fragment thereof.

The inventors then subjected the protein sequence of SEQ ID No: 254 to codon optimisation for human expression, and one embodiment of the codon optimised nucleic acid (DNA) sequence that includes a start (ATG) and a stop (TGA) codon is provided herein as SEQ ID No: 255, as follows:

[SEQ ID No: 255]
ATGGAAAGATCCCCTGACGTGTCCCCTGGACCTAGCAGAAGCTTC
AAAGAGGAACTGCTCTGCGCCGTGTGCTACGACCCCTTCAGAGAT
GCCGTGACACTGAGATGCGGCCACAACTTCTGCAGAGGCTGCGTG
TCCAGATGCTGGGAAGTGCAGGTTTCCCCTACATGCCCCGTGTGC
AAGGACAGAGCCTCTCCTGCCGATCTGCGGACCAATCACACCCTG
AACAACCTGGTGGAAAAGCTGCTGAGAGAAGAGGCCGAAGGCGCC
AGATGGACCAGCTACAGATTCAGCAGAGTGTGCCGGCTGCACAGA
GGCCAGCTGAGCCTGTTCTGTCTCGAGGACAAAGAACTGCTGTGC
TGCAGCTGCCAGGCCGATCCTAGACACCAGGGACATAGAGTGCAG
CCCGTGAAGGACACAGCCCACGACTTCAGAGCCAAGTGCCGGAAC
ATGGAACACGCCCTGAGAGAGAAGGCCAAAGCCTTCTGGGCCATG
CGGAGAAGCTATGAGGCCATTGCCAAGCACAATCAGGTGGAAGCC
GCCTGGCTGGAAGGCCGGATCAGACAAGAGTTCGACAAGCTGCGC
GAGTTCCTGAGAGTGGAAGAACAGGCCATCCTGGACGCCATGGCC
GAGGAAACAAGACAGAAACAGCTGCTGGCCGACGAGAAGATGAAG
CAGCTGACCGAAGAGACAGAGGTGCTGGCCCACGAAATCGAGCGG
CTGCAGATGGAAATGAAGGAAGATGATGTGTCCTTTCTGATGAAG
CACAAGAGCCGGAAGCGGCGGCTGTTCTGCACAATGGAACCTGAG
CCAGTGCAGCCTGGCATGCTGATCGATGTGTGCAAGTACCTGGGC
AGCCTGCAGTACAGAGTGTGGAAGAAAATGCTGGCCTCCGTGGAA
AGCGTGCCCTTCAGCTTCGACCCTAATACTGCCGCTGGCTGGCTG
AGCGTGTCCGATGATCTGACCAGCGTGACCAACCACGGCTACAGA
GTGCAGGTCGAGAACCCCGAGAGATTCAGCTCTGCCCCTTGTCTG
CTGGGCTCCAGAGTGTTTTCTCAGGGCTCTCACGCCTGGGAAGTT
GCCCTTGGAGGACTCCAGTCTTGGAGAGTGGGCGTTGTCAGAGTG
CGGCAGGATTCTGGCGCCGAAGGACACTCTCACAGCTGCTACCAC
GATACCCGCAGCGGCTTTTGGTACGTGTGTAGAACACAGGGCGTC
GAGGGCGACCACTGTGTGACATCTGACCCTGCCACATCTCCTCTG
GTGCTGGCTATCCCTCGGAGACTGAGAGTCGAGCTGGAATGCGAG
GAAGGCGAGCTGAGCTTCTACGACGCCGAGAGACACTGCCACCTG
TACACCTTCCACGCCAGATTTGGCGAAGTGCGGCCCTACTTTTAT
CTCGGCGGAGCTAGAGGTGCCGGACCTCCTGAACCTCTGAGAATC
TGCCCTCTGCACATCAGCGTGAAAGAGGAATTGGACGGCTGA

Hence, preferably the RNA construct is encoded by a DNA sequence substantially as set out in SEQ ID No: 255, or a fragment or variant thereof.

In an embodiment, the RNA sequence corresponding to the codon optimised DNA sequence of SEQ ID No: 255 that includes a start (AUG) and a stop (UGA) codon is provided herein as SEQ ID No: 256, as follows:

[SEQ ID No: 256]
AUGGAAAGAUCCCCUGACGUGUCCCCUGGACCUAGCAGAAGCUUC
AAAGAGGAACUGCUCUGCGCCGUGUGCUACGACCCCUUCAGAGAU
GCCGUGACACUGAGAUGCGGCCACAACUUCUGCAGAGGCUGCGUG
UCCAGAUGCUGGGAAGUGCAGGUUUCCCCUACAUGCCCCGUGUGC
AAGGACAGAGCCUCUCCUGCCGAUCUGCGGACCAAUCACACCCUG
AACAACCUGGUGGAAAAGCUGCUGAGAGAAGAGGCCGAAGGCGCC
AGAUGGACCAGCUACAGAUUCAGCAGAGUGUGCCGGCUGCACAGA
GGCCAGCUGAGCCUGUUCUGUCUCGAGGACAAAGAACUGCUGUGC
UGCAGCUGCCAGGCCGAUCCUAGACACCAGGGACAUAGAGUGCAG
CCCGUGAAGGACACAGCCCACGACUUCAGAGCCAAGUGCCGGAAC
AUGGAACACGCCCUGAGAGAGAAGGCCAAAGCCUUCUGGGCCAUG
CGGAGAAGCUAUGAGGCCAUUGCCAAGCACAAUCAGGUGGAAGCC
GCCUGGCUGGAAGGCCGGAUCAGACAAGAGUUCGACAAGCUGCGC
GAGUUCCUGAGAGUGGAAGAACAGGCCAUCCUGGACGCCAUGGCC
GAGGAAACAAGACAGAAACAGCUGCUGGCCGACGAGAAGAUGAAG
CAGCUGACCGAAGAGACAGAGGUGCUGGCCCACGAAAUCGAGCGG
CUGCAGAUGGAAAUGAAGGAAGAUGAUGUGUCCUUUCUGAUGAAG
CACAAGAGCCGGAAGCGGCGGCUGUUCUGCACAAUGGAACCUGAG
CCAGUGCAGCCUGGCAUGCUGAUCGAUGUGUGCAAGUACCUGGGC
AGCCUGCAGUACAGAGUGUGGAAGAAAAUGCUGGCCUCCGUGGAA
AGCGUGCCCUUCAGCUUCGACCCUAAUACUGCCGCUGGCUGGCUG
AGCGUGUCCGAUGAUCUGACCAGCGUGACCAACCACGGCUACAGA
GUGCAGGUCGAGAACCCCGAGAGAUUCAGCUCUGCCCCUUGUCUG
CUGGGCUCCAGAGUGUUUUCUCAGGGCUCUCACGCCUGGGAAGUU
GCCCUUGGAGGACUCCAGUCUUGGAGAGUGGGCGUUGUCAGAGUG
CGGCAGGAUUCUGGCGCCGAAGGACACUCUCACAGCUGCUACCAC
GAUACCCGCAGCGGCUUUUGGUACGUGUGUAGAACACAGGGCGUC
GAGGGCGACCACUGUGUGACAUCUGACCCUGCCACAUCUCCUCUG
GUGCUGGCUAUCCCUCGGAGACUGAGAGUCGAGCUGGAAUGCGAG
GAAGGCGAGCUGAGCUUCUACGACGCCGAGAGACACUGCCACCUG
UACACCUUCCACGCCAGAUUUGGCGAAGUGCGGCCCUACUUUUAU
CUCGGCGGAGCUAGAGGUGCCGGACCUCCUGAACCUCUGAGAAUC
UGCCCUCUGCACAUCAGCGUGAAAGAGGAAUUGGACGGCUGA

Hence, preferably the RNA construct comprises a sequence substantially as set out in SEQ ID No: 256, or a fragment or variant thereof.

Category 3: Inhibitors of Interferon Signalling

In another embodiment, the IMP may be configured to inhibit interferon signalling.

Thus, the reduction, ablation or blocking of the innate immune response to RNA is preferably achieved by the IMP by inhibiting the signalling of interferon which leads to the production of interferon stimulated genes (e.g. IFIT1) which impact on the activity of RNA. Preferably, therefore, the innate modulatory protein encoded by the RNA construct comprises a protein/inhibitor or a mutated or non-functional protein of the interferon signalling pathway, or a dominant negative acting form thereof.

In one embodiment, the inhibitor of an innate signalling pathway, or a dominant negative acting form thereof, is STAT1 dominant negative form. STAT1 (NCBI Reference Sequence: NM_007315.4; UniProtKB—P42224 (STAT1_HUMAN)), or an orthologue thereof may be rendered dominant negative by a Y701F mutation that can act in a dominant negative fashion to block ISGF-3 complex formation, and is represented herein as SEQ ID No: 66, as follows:

[SEQ ID No: 66]
MSQWYELQQLDSKFLEQVHQLYDDSFPMEIRQYLAQWLEKQDWEH
AANDVSFATIRFHDLLSQLDDQYSRFSLENNFLLQHNIRKSKRNL
QDNFQEDPIQMSMIIYSCLKEERKILENAQRFNQAQSGNIQSTVM
LDKQKELDSKVRNVKDKVMCIEHEIKSLEDLQDEYDFKCKTLQNR
EHETNGVAKSDQKQEQLLLKKMYLMLDNKRKEVVHKIIELLNVTE
LTQNALINDELVEWKRRQQSACIGGPPNACLDQLQNWFTIVAESL
QQVRQQLKKLEELEQKYTYEHDPITKNKQVLWDRTFSLFQQLIQS
SFVVERQPCMPTHPQRPLVLKTGVQFTVKLRLLVKLQELNYNLKV
KVLFDKDVNERNTVKGFRKFNILGTHTKVMNMEESTNGSLAAEFR
HLQLKEQKNAGTRTNEGPLIVTEELHSLSFETQLCQPGLVIDLET
TSLPVVVISNVSQLPSGWASILWYNMLVAEPRNLSFFLTPPCARW
AQLSEVLSWQFSSVTKRGLNVDQLNMLGEKLLGPNASPDGLIPWT
RFCKENINDKNFPFWLWIESILELIKKHLLPLWNDGCIMGFISKE
RERALLKDQQPGTFLLRFSESSREGAITFTWVERSQNGGEPDFHA
VEPYTKKELSAVTFPDIIRNYKVMAAENIPENPLKYLYPNIDKDH
AFGKYYSRPKEAPEPMELDGPKGTGFIKTELISVSEVHPSRLQTT
DNLLPMSPEEFDEVSRIVGSVEFDSMMNTV

Therefore, preferably the RNA construct of the first aspect comprises a nucleotide sequence which encodes an amino acid sequence substantially as set out in SEQ ID No: 66, or a variant or fragment thereof.

In one embodiment, the STAT1 dominant negative form polypeptide is encoded by the DNA nucleotide sequence of SEQ ID No: 67, as follows:

[SEQ ID No: 67]
ATGTCTCAGTGGTACGAACTTCAGCAGCTTGACTCAAAATTCCTG
GAGCAGGTTCACCAGCTTTATGATGACAGTTTTCCCATGGAAATC
AGACAGTACCTGGCACAGTGGTTAGAAAAGCAAGACTGGGAGCAC
GCTGCCAATGATGTTTCATTTGCCACCATCCGTTTTCATGACCTC
CTGTCACAGCTGGATGATCAATATAGTCGCTTTTCTTTGGAGAAT
AACTTCTTGCTACAGCATAACATAAGGAAAAGCAAGCGTAATCTT
CAGGATAATTTTCAGGAAGACCCAATCCAGATGTCTATGATCATT
TACAGCTGTCTGAAGGAAGAAAGGAAAATTCTGGAAAACGCCCAG
AGATTTAATCAGGCTCAGTCGGGGAATATTCAGAGCACAGTGATG
TTAGACAAACAGAAAGAGCTTGACAGTAAAGTCAGAAATGTGAAG
GACAAGGTTATGTGTATAGAGCATGAAATCAAGAGCCTGGAAGAT
TTACAAGATGAATATGACTTCAAATGCAAAACCTTGCAGAACAGA
GAACACGAGACCAATGGTGTGGCAAAGAGTGATCAGAAACAAGAA
CAGCTGTTACTCAAGAAGATGTATTTAATGCTTGACAATAAGAGA
AAGGAAGTAGTTCACAAAATAATAGAGTTGCTGAATGTCACTGAA
CTTACCCAGAATGCCCTGATTAATGATGAACTAGTGGAGTGGAAG
CGGAGACAGCAGAGCGCCTGTATTGGGGGGCCGCCCAATGCTTGC
TTGGATCAGCTGCAGAACTGGTTCACTATAGTTGCGGAGAGTCTG
CAGCAAGTTCGGCAGCAGCTTAAAAAGTTGGAGGAATTGGAACAG
AAATACACCTACGAACATGACCCTATCACAAAAAACAAACAAGTG
TTATGGGACCGCACCTTCAGTCTTTTCCAGCAGCTCATTCAGAGC
TCGTTTGTGGTGGAAAGACAGCCCTGCATGCCAACGCACCCTCAG
AGGCCGCTGGTCTTGAAGACAGGGGTCCAGTTCACTGTGAAGTTG
AGACTGTTGGTGAAATTGCAAGAGCTGAATTATAATTTGAAAGTC
AAAGTCTTATTTGATAAAGATGTGAATGAGAGAAATACAGTAAAA
GGATTTAGGAAGTTCAACATTTTGGGCACGCACACAAAAGTGATG
AACATGGAGGAGTCCACCAATGGCAGTCTGGCGGCTGAATTTCGG
CACCTGCAATTGAAAGAACAGAAAAATGCTGGCACCAGAACGAAT
GAGGGTCCTCTCATCGTTACTGAAGAGCTTCACTCCCTTAGTTTT
GAAACCCAATTGTGCCAGCCTGGTTTGGTAATTGACCTCGAGACG
ACCTCTCTGCCCGTTGTGGTGATCTCCAACGTCAGCCAGCTCCCG
AGCGGTTGGGCCTCCATCCTTTGGTACAACATGCTGGTGGCGGAA
CCCAGGAATCTGTCCTTCTTCCTGACTCCACCATGTGCACGATGG
GCTCAGCTTTCAGAAGTGCTGAGTTGGCAGTTTTCTTCTGTCACC
AAAAGAGGTCTCAATGTGGACCAGCTGAACATGTTGGGAGAGAAG
CTTCTTGGTCCTAACGCCAGCCCCGATGGTCTCATTCCGTGGACG
AGGTTTTGTAAGGAAAATATAAATGATAAAAATTTTCCCTTCTGG
CTTTGGATTGAAAGCATCCTAGAACTCATTAAAAAACACCTGCTC
CCTCTCTGGAATGATGGGTGCATCATGGGCTTCATCAGCAAGGAG
CGAGAGCGTGCCCTGTTGAAGGACCAGCAGCCGGGGACCTTCCTG
CTGCGGTTCAGTGAGAGCTCCCGGGAAGGGGCCATCACATTCACA
TGGGTGGAGCGGTCCCAGAACGGAGGCGAACCTGACTTCCATGCG
GTTGAACCCTACACGAAGAAAGAACTTTCTGCTGTTACTTTCCCT
GACATCATTCGCAATTACAAAGTCATGGCTGCTGAGAATATTCCT
GAGAATCCCCTGAAGTATCTGTATCCAAATATTGACAAAGACCAT
GCCTTTGGAAAGTATTACTCCAGGCCAAAGGAAGCACCAGAGCCA
ATGGAACTTGATGGCCCTAAAGGAACTGGATTTATCAAGACTGAG
TTGATTTCTGTGTCTGAAGTTCACCCTTCTAGACTTCAGACCACA
GACAACCTGCTCCCCATGTCTCCTGAGGAGTTTGACGAGGTGTCT
CGGATAGTGGGCTCTGTAGAATTCGACAGTATGATGAACACAGTA

Accordingly, preferably the STAT1 dominant negative form polypeptide is encoded by the DNA nucleotide sequence substantially as set out in SEQ ID NO: 67, or a variant or fragment thereof.

Thus, the RNA construct may comprise an RNA nucleotide sequence of SEQ ID No: 68, as follows:

[SEQ ID No: 68]
AUGUCUCAGUGGUACGAACUUCAGCAGCUUGACUCAAAAUUCCUG
GAGCAGGUUCACCAGCUUUAUGAUGACAGUUUUCCCAUGGAAAUC
AGACAGUACCUGGCACAGUGGUUAGAAAAGCAAGACUGGGAGCAC
GCUGCCAAUGAUGUUUCAUUUGCCACCAUCCGUUUUCAUGACCUC
CUGUCACAGCUGGAUGAUCAAUAUAGUCGCUUUUCUUUGGAGAAU
AACUUCUUGCUACAGCAUAACAUAAGGAAAAGCAAGCGUAAUCUU
CAGGAUAAUUUUCAGGAAGACCCAAUCCAGAUGUCUAUGAUCAUU
UACAGCUGUCUGAAGGAAGAAAGGAAAAUUCUGGAAAACGCCCAG
AGAUUUAAUCAGGCUCAGUCGGGGAAUAUUCAGAGCACAGUGAUG
UUAGACAAACAGAAAGAGCUUGACAGUAAAGUCAGAAAUGUGAAG
GACAAGGUUAUGUGUAUAGAGCAUGAAAUCAAGAGCCUGGAAGAU
UUACAAGAUGAAUAUGACUUCAAAUGCAAAACCUUGCAGAACAGA
GAACACGAGACCAAUGGUGUGGCAAAGAGUGAUCAGAAACAAGAA
CAGCUGUUACUCAAGAAGAUGUAUUUAAUGCUUGACAAUAAGAGA
AAGGAAGUAGUUCACAAAAUAAUAGAGUUGCUGAAUGUCACUGAA
CUUACCCAGAAUGCCCUGAUUAAUGAUGAACUAGUGGAGUGGAAG
CGGAGACAGCAGAGCGCCUGUAUUGGGGGGCCGCCCAAUGCUUGC
UUGGAUCAGCUGCAGAACUGGUUCACUAUAGUUGCGGAGAGUCUG
CAGCAAGUUCGGCAGCAGCUUAAAAAGUUGGAGGAAUUGGAACAG
AAAUACACCUACGAACAUGACCCUAUCACAAAAAACAAACAAGUG
UUAUGGGACCGCACCUUCAGUCUUUUCCAGCAGCUCAUUCAGAGC
UCGUUUGUGGUGGAAAGACAGCCCUGCAUGCCAACGCACCCUCAG
AGGCCGCUGGUCUUGAAGACAGGGGUCCAGUUCACUGUGAAGUUG
AGACUGUUGGUGAAAUUGCAAGAGCUGAAUUAUAAUUUGAAAGUC
AAAGUCUUAUUUGAUAAAGAUGUGAAUGAGAGAAAUACAGUAAAA
GGAUUUAGGAAGUUCAACAUUUUGGGCACGCACACAAAAGUGAUG
AACAUGGAGGAGUCCACCAAUGGCAGUCUGGCGGCUGAAUUUCGG
CACCUGCAAUUGAAAGAACAGAAAAAUGCUGGCACCAGAACGAAU
GAGGGUCCUCUCAUCGUUACUGAAGAGCUUCACUCCCUUAGUUUU
GAAACCCAAUUGUGCCAGCCUGGUUUGGUAAUUGACCUCGAGACG
ACCUCUCUGCCCGUUGUGGUGAUCUCCAACGUCAGCCAGCUCCCG
AGCGGUUGGGCCUCCAUCCUUUGGUACAACAUGCUGGUGGCGGAA
CCCAGGAAUCUGUCCUUCUUCCUGACUCCACCAUGUGCACGAUGG
GCUCAGCUUUCAGAAGUGCUGAGUUGGCAGUUUUCUUCUGUCACC
AAAAGAGGUCUCAAUGUGGACCAGCUGAACAUGUUGGGAGAGAAG
CUUCUUGGUCCUAACGCCAGCCCCGAUGGUCUCAUUCCGUGGACG
AGGUUUUGUAAGGAAAAUAUAAAUGAUAAAAAUUUUCCCUUCUGG
CUUUGGAUUGAAAGCAUCCUAGAACUCAUUAAAAAACACCUGCUC
CCUCUCUGGAAUGAUGGGUGCAUCAUGGGCUUCAUCAGCAAGGAG
CGAGAGCGUGCCCUGUUGAAGGACCAGCAGCCGGGGACCUUCCUG
CUGCGGUUCAGUGAGAGCUCCCGGGAAGGGGCCAUCACAUUCACA
UGGGUGGAGCGGUCCCAGAACGGAGGCGAACCUGACUUCCAUGCG
GUUGAACCCUACACGAAGAAAGAACUUUCUGCUGUUACUUUCCCU
GACAUCAUUCGCAAUUACAAAGUCAUGGCUGCUGAGAAUAUUCCU
GAGAAUCCCCUGAAGUAUCUGUAUCCAAAUAUUGACAAAGACCAU
GCCUUUGGAAAGUAUUACUCCAGGCCAAAGGAAGCACCAGAGCCA
AUGGAACUUGAUGGCCCUAAAGGAACUGGAUUUAUCAAGACUGAG
UUGAUUUCUGUGUCUGAAGUUCACCCUUCUAGACUUCAGACCACA
GACAACCUGCUCCCCAUGUCUCCUGAGGAGUUUGACGAGGUGUCU
CGGAUAGUGGGCUCUGUAGAAUUCGACAGUAUGAUGAACACAGUA

Therefore, preferably the RNA construct comprises an RNA nucleotide sequence substantially as set out in SEQ ID No: 68, or a variant or fragment thereof.

The inventors then subjected the protein sequence of SEQ ID No: 66 to codon optimisation for human expression, and one embodiment of the codon optimised nucleic acid (DNA) sequence that includes a start (ATG) and a stop (TGA) codon is provided herein as SEQ ID No: 69, as follows:

[SEQ ID No: 69]
ATGAGCCAGTGGTACGAGCTGCAGCAGCTGGACAGCAAGTTCCTG
GAACAGGTGCACCAGCTGTACGACGACAGCTTCCCCATGGAAATC
CGGCAGTATCTGGCCCAGTGGCTGGAAAAGCAGGATTGGGAACAC
GCCGCCAACGACGTGTCCTTCGCCACCATCAGATTCCACGACCTG
CTGAGCCAGCTGGACGACCAGTACAGCAGATTCAGCCTGGAAAAC
AACTTCCTGCTCCAGCACAACATCCGGAAGTCCAAGCGGAACCTG
CAGGACAACTTCCAAGAGGACCCCATCCAGATGTCCATGATCATC
TACAGCTGCCTGAAAGAGGAACGGAAGATCCTGGAAAATGCCCAG
CGGTTCAATCAGGCCCAGAGCGGCAATATCCAGAGCACCGTGATG
CTGGACAAGCAGAAAGAACTGGACTCCAAAGTGCGGAACGTCAAG
GACAAAGTGATGTGCATCGAGCACGAGATCAAGAGCCTGGAAGAT
CTGCAGGACGAGTACGACTTCAAGTGCAAGACCCTGCAGAACCGG
GAACACGAGACAAACGGCGTGGCCAAGAGCGACCAGAAGCAAGAA
CAGCTGCTCCTGAAGAAAATGTACCTGATGCTCGACAACAAACGG
AAAGAGGTGGTCCACAAGATCATCGAGCTGCTGAACGTGACCGAG
CTGACCCAGAACGCCCTGATCAACGACGAGCTGGTGGAATGGAAG
CGGAGACAGCAGTCTGCCTGTATCGGCGGACCTCCTAATGCCTGC
CTGGACCAGCTGCAGAACTGGTTCACAATCGTGGCCGAGAGCCTG
CAGCAAGTGCGCCAGCAGCTGAAGAAGCTGGAAGAACTCGAGCAG
AAGTACACCTACGAGCACGACCCCATCACCAAGAACAAACAGGTG
CTGTGGGACAGAACCTTCAGCCTGTTCCAACAGCTGATCCAGTCC
AGCTTCGTGGTGGAAAGACAGCCCTGCATGCCTACACACCCTCAG
AGGCCACTGGTGCTGAAAACCGGCGTGCAGTTCACCGTGAAGCTG
CGGCTGCTGGTCAAGCTGCAAGAGCTGAACTACAACCTGAAAGTG
AAGGTGCTGTTCGACAAGGACGTGAACGAGCGGAACACCGTGAAA
GGCTTCCGCAAGTTCAACATCCTGGGCACCCACACAAAAGTGATG
AACATGGAAGAGAGCACCAACGGCAGCCTGGCCGCCGAGTTTAGA
CACCTCCAGCTGAAAGAGCAGAAGAACGCCGGCACCAGGACCAAT
GAGGGACCTCTGATCGTGACAGAGGAACTGCACAGCCTGAGCTTC
GAAACCCAGCTGTGTCAGCCAGGCCTCGTGATCGATCTGGAAACC
ACAAGCCTGCCTGTGGTGGTCATCAGCAATGTGTCCCAGCTGCCT
TCTGGCTGGGCCAGCATCCTGTGGTACAACATGCTGGTGGCCGAG
CCTCGGAACCTGTCCTTCTTTCTGACCCCTCCATGTGCCAGATGG
GCCCAGCTGTCTGAAGTGCTGAGCTGGCAGTTTAGCAGCGTGACC
AAGAGGGGCCTGAATGTCGACCAGCTGAATATGCTGGGCGAGAAG
CTGCTGGGCCCCAACGCTTCTCCTGATGGACTGATCCCTTGGACC
AGATTCTGCAAAGAGAATATCAACGACAAGAACTTCCCGTTCTGG
CTGTGGATCGAGAGCATCCTGGAACTGATCAAGAAACATCTGCTG
CCCCTGTGGAACGACGGCTGCATCATGGGCTTCATCTCCAAAGAG
AGAGAGCGGGCCCTGCTGAAGGATCAGCAGCCAGGCACATTCCTG
CTGCGGTTTAGCGAGTCTAGCAGAGAGGGCGCCATCACCTTTACC
TGGGTCGAGAGATCTCAGAACGGCGGCGAGCCTGATTTTCACGCC
GTGGAACCCTACACCAAAAAAGAACTGAGCGCCGTGACATTCCCC
GACATCATCCGGAACTACAAAGTCATGGCCGCTGAGAATATCCCC
GAGAATCCCCTGAAGTATCTGTACCCCAACATCGATAAGGACCAC
GCCTTCGGCAAGTACTACAGCAGACCCAAAGAGGCCCCTGAGCCT
ATGGAACTGGATGGCCCTAAAGGCACCGGCTTCATCAAGACAGAG
CTGATCTCCGTGTCCGAGGTGCACCCTAGCAGACTGCAGACCACC
GATAACCTGCTGCCTATGAGCCCCGAGGAATTCGACGAGGTGTCC
AGAATCGTGGGCAGCGTGGAATTCGATAGCATGATGAATACCGTG
TGA

Hence, preferably the RNA construct is encoded by a DNA sequence substantially as set out in SEQ ID No: 69, or a fragment or variant thereof.

In an embodiment, the RNA sequence corresponding to the codon optimised DNA sequence of SEQ ID No: 69 that includes a start (AUG) and a stop (UGA) codon is provided herein as SEQ ID No: 70, as follows:

[SEQ ID No: 70]
AUGAGCCAGUGGUACGAGCUGCAGCAGCUGGACAGCAAGUUCCUG
GAACAGGUGCACCAGCUGUACGACGACAGCUUCCCCAUGGAAAUC
CGGCAGUAUCUGGCCCAGUGGCUGGAAAAGCAGGAUUGGGAACAC
GCCGCCAACGACGUGUCCUUCGCCACCAUCAGAUUCCACGACCUG
CUGAGCCAGCUGGACGACCAGUACAGCAGAUUCAGCCUGGAAAAC
AACUUCCUGCUCCAGCACAACAUCCGGAAGUCCAAGCGGAACCUG
CAGGACAACUUCCAAGAGGACCCCAUCCAGAUGUCCAUGAUCAUC
UACAGCUGCCUGAAAGAGGAACGGAAGAUCCUGGAAAAUGCCCAG
CGGUUCAAUCAGGCCCAGAGCGGCAAUAUCCAGAGCACCGUGAUG
CUGGACAAGCAGAAAGAACUGGACUCCAAAGUGCGGAACGUCAAG
GACAAAGUGAUGUGCAUCGAGCACGAGAUCAAGAGCCUGGAAGAU
CUGCAGGACGAGUACGACUUCAAGUGCAAGACCCUGCAGAACCGG
GAACACGAGACAAACGGCGUGGCCAAGAGCGACCAGAAGCAAGAA
CAGCUGCUCCUGAAGAAAAUGUACCUGAUGCUCGACAACAAACGG
AAAGAGGUGGUCCACAAGAUCAUCGAGCUGCUGAACGUGACCGAG
CUGACCCAGAACGCCCUGAUCAACGACGAGCUGGUGGAAUGGAAG
CGGAGACAGCAGUCUGCCUGUAUCGGCGGACCUCCUAAUGCCUGC
CUGGACCAGCUGCAGAACUGGUUCACAAUCGUGGCCGAGAGCCUG
CAGCAAGUGCGCCAGCAGCUGAAGAAGCUGGAAGAACUCGAGCAG
AAGUACACCUACGAGCACGACCCCAUCACCAAGAACAAACAGGUG
CUGUGGGACAGAACCUUCAGCCUGUUCCAACAGCUGAUCCAGUCC
AGCUUCGUGGUGGAAAGACAGCCCUGCAUGCCUACACACCCUCAG
AGGCCACUGGUGCUGAAAACCGGCGUGCAGUUCACCGUGAAGCUG
CGGCUGCUGGUCAAGCUGCAAGAGCUGAACUACAACCUGAAAGUG
AAGGUGCUGUUCGACAAGGACGUGAACGAGCGGAACACCGUGAAA
GGCUUCCGCAAGUUCAACAUCCUGGGCACCCACACAAAAGUGAUG
AACAUGGAAGAGAGCACCAACGGCAGCCUGGCCGCCGAGUUUAGA
CACCUCCAGCUGAAAGAGCAGAAGAACGCCGGCACCAGGACCAAU
GAGGGACCUCUGAUCGUGACAGAGGAACUGCACAGCCUGAGCUUC
GAAACCCAGCUGUGUCAGCCAGGCCUCGUGAUCGAUCUGGAAACC
ACAAGCCUGCCUGUGGUGGUCAUCAGCAAUGUGUCCCAGCUGCCU
UCUGGCUGGGCCAGCAUCCUGUGGUACAACAUGCUGGUGGCCGAG
CCUCGGAACCUGUCCUUCUUUCUGACCCCUCCAUGUGCCAGAUGG
GCCCAGCUGUCUGAAGUGCUGAGCUGGCAGUUUAGCAGCGUGACC
AAGAGGGGCCUGAAUGUCGACCAGCUGAAUAUGCUGGGCGAGAAG
CUGCUGGGCCCCAACGCUUCUCCUGAUGGACUGAUCCCUUGGACC
AGAUUCUGCAAAGAGAAUAUCAACGACAAGAACUUCCCGUUCUGG
CUGUGGAUCGAGAGCAUCCUGGAACUGAUCAAGAAACAUCUGCUG
CCCCUGUGGAACGACGGCUGCAUCAUGGGCUUCAUCUCCAAAGAG
AGAGAGCGGGCCCUGCUGAAGGAUCAGCAGCCAGGCACAUUCCUG
CUGCGGUUUAGCGAGUCUAGCAGAGAGGGCGCCAUCACCUUUACC
UGGGUCGAGAGAUCUCAGAACGGCGGCGAGCCUGAUUUUCACGCC
GUGGAACCCUACACCAAAAAAGAACUGAGCGCCGUGACAUUCCCC
GACAUCAUCCGGAACUACAAAGUCAUGGCCGCUGAGAAUAUCCCC
GAGAAUCCCCUGAAGUAUCUGUACCCCAACAUCGAUAAGGACCAC
GCCUUCGGCAAGUACUACAGCAGACCCAAAGAGGCCCCUGAGCCU
AUGGAACUGGAUGGCCCUAAAGGCACCGGCUUCAUCAAGACAGAG
CUGAUCUCCGUGUCCGAGGUGCACCCUAGCAGACUGCAGACCACC
GAUAACCUGCUGCCUAUGAGCCCCGAGGAAUUCGACGAGGUGUCC
AGAAUCGUGGGCAGCGUGGAAUUCGAUAGCAUGAUGAAUACCGUG
UGA

Hence, preferably the RNA construct comprises a sequence substantially as set out in SEQ ID No: 70, or a fragment or variant thereof.

In one embodiment, the inhibitor of an innate signalling pathway, or a dominant negative acting form thereof, is STAT2 short form, which binds to IRF9. One embodiment of the STAT2 dominant negative short form is referred to as STAT2 (133-315) NCBI Reference Sequence: NM_005419.4; UniProtKB—P52630 (STAT2_HUMAN), or an orthologue thereof, and is represented herein as SEQ ID No: 71, as follows:

[SEQ ID No: 71]
VLETPVESQQHEIESRILDLRAMMEKLVKSISQLKDQQDVFCFRY
KIQAKGKTPSLDPHQTKEQKILQETLNELDKRRKEVLDASKALLG
RLTTLIELLLPKLEEWKAQQQKACIRAPIDHGLEQLETWFTAGAK
LLFHLRQLLKELKGLSCLVSYQDDPLTKGVDLRNAQVTELLQRLL
HRA

Therefore, preferably the RNA construct of the first aspect comprises a nucleotide sequence which encodes an amino acid sequence substantially as set out in SEQ ID No: 71, or a variant or fragment thereof.

In one embodiment, the STAT2 short form polypeptide is encoded by the DNA nucleotide sequence of SEQ ID No: 72, as follows:

[SEQ ID No: 72]
GTTCTCGAAACACCTGTGGAGAGCCAGCAACATGAGATTGAATCC
CGGATCCTGGATTTAAGGGCTATGATGGAGAAGCTGGTAAAATCC
ATCAGCCAACTGAAAGACCAGCAGGATGTCTTCTGCTTCCGATAT
AAGATCCAGGCCAAAGGGAAGACACCCTCTCTGGACCCCCATCAG
ACCAAAGAGCAGAAGATTCTGCAGGAAACTCTCAATGAACTGGAC
AAAAGGAGAAAGGAGGTGCTGGATGCCTCCAAAGCACTGCTAGGC
CGATTAACTACCCTAATCGAGCTACTGCTGCCAAAGTTGGAGGAG
TGGAAGGCCCAGCAGCAAAAAGCCTGCATCAGAGCTCCCATTGAC
CACGGGTTGGAACAGCTGGAGACATGGTTCACAGCTGGAGCAAAG
CTGTTGTTTCACCTGAGGCAGCTGCTGAAGGAGCTGAAGGGACTG
AGTTGCCTGGTTAGCTATCAGGATGACCCTCTGACCAAAGGGGTG
GACCTACGCAACGCCCAGGTCACAGAGTTGCTACAGCGTCTGCTC
CACAGAGCC

Accordingly, preferably the STAT2 short form polypeptide is encoded by the DNA nucleotide sequence substantially as set out in SEQ ID NO: 72, or a variant or fragment thereof.

Thus, the RNA construct may comprise an RNA nucleotide sequence of SEQ ID No: 73, as follows:

[SEQ ID No: 73]
GUUCUCGAAACACCUGUGGAGAGCCAGCAACAUGAGAUUGAAUCC
CGGAUCCUGGAUUUAAGGGCUAUGAUGGAGAAGCUGGUAAAAUCC
AUCAGCCAACUGAAAGACCAGCAGGAUGUCUUCUGCUUCCGAUAU
AAGAUCCAGGCCAAAGGGAAGACACCCUCUCUGGACCCCCAUCAG
ACCAAAGAGCAGAAGAUUCUGCAGGAAACUCUCAAUGAACUGGAC
AAAAGGAGAAAGGAGGUGCUGGAUGCCUCCAAAGCACUGCUAGGC
CGAUUAACUACCCUAAUCGAGCUACUGCUGCCAAAGUUGGAGGAG
UGGAAGGCCCAGCAGCAAAAAGCCUGCAUCAGAGCUCCCAUUGAC
CACGGGUUGGAACAGCUGGAGACAUGGUUCACAGCUGGAGCAAAG
CUGUUGUUUCACCUGAGGCAGCUGCUGAAGGAGCUGAAGGGACUG
AGUUGCCUGGUUAGCUAUCAGGAUGACCCUCUGACCAAAGGGGUG
GACCUACGCAACGCCCAGGUCACAGAGUUGCUACAGCGUCUGCUC
CACAGAGCC

Therefore, preferably the RNA construct comprises an RNA nucleotide sequence substantially as set out in SEQ ID No: 73, or a variant or fragment thereof.

The inventors then subjected the protein sequence of SEQ ID No: 71 to codon optimisation for human expression, and one embodiment of the codon optimised nucleic acid (DNA) sequence that includes a start (ATG) and a stop (TGA) codon is provided herein as SEQ ID No: 74, as follows:

[SEQ ID No: 74]
ATGGTGCTGGAAACCCCTGTGGAAAGCCAGCAGCACGAGATCGAG
AGCAGAATCCTGGACCTGCGGGCCATGATGGAAAAGCTGGTCAAG
AGCATCAGCCAGCTGAAGGACCAGCAGGACGTGTTCTGCTTCCGG
TACAAGATCCAGGCCAAGGGCAAGACCCCTAGCCTGGATCCTCAC
CAGACCAAAGAGCAGAAGATCCTGCAAGAGACACTGAACGAGCTG
GACAAGCGGCGGAAAGAAGTGCTGGACGCCTCTAAAGCTCTGCTG
GGCAGACTGACCACTCTGATCGAACTGCTGCTGCCCAAGCTGGAA
GAGTGGAAGGCCCAGCAACAGAAGGCCTGCATCAGAGCCCCTATC
GACCACGGACTGGAACAGCTGGAAACATGGTTTACCGCTGGCGCC
AAGCTGCTGTTCCACCTGAGACAGCTGCTGAAAGAGCTGAAGGGC
CTGAGCTGCCTGGTGTCCTACCAGGATGACCCTCTGACCAAAGGC
GTGGACCTGAGAAACGCCCAAGTGACCGAACTGCTCCAGCGGCTG
CTGCATAGAGCTTGA

Hence, preferably the RNA construct is encoded by a DNA sequence substantially as set out in SEQ ID No: 74, or a fragment or variant thereof.

In an embodiment, the RNA sequence corresponding to the codon optimised DNA sequence of SEQ ID No: 74 that includes a start (AUG) and a stop (UGA) codon is provided herein as SEQ ID No: 75, as follows:

[SEQ ID No: 75]
AUGGUGCUGGAAACCCCUGUGGAAAGCCAGCAGCACGAGAUCGAG
AGCAGAAUCCUGGACCUGCGGGCCAUGAUGGAAAAGCUGGUCAAG
AGCAUCAGCCAGCUGAAGGACCAGCAGGACGUGUUCUGCUUCCGG
UACAAGAUCCAGGCCAAGGGCAAGACCCCUAGCCUGGAUCCUCAC
CAGACCAAAGAGCAGAAGAUCCUGCAAGAGACACUGAACGAGCUG
GACAAGCGGCGGAAAGAAGUGCUGGACGCCUCUAAAGCUCUGCUG
GGCAGACUGACCACUCUGAUCGAACUGCUGCUGCCCAAGCUGGAA
GAGUGGAAGGCCCAGCAACAGAAGGCCUGCAUCAGAGCCCCUAUC
GACCACGGACUGGAACAGCUGGAAACAUGGUUUACCGCUGGCGCC
AAGCUGCUGUUCCACCUGAGACAGCUGCUGAAAGAGCUGAAGGGC
CUGAGCUGCCUGGUGUCCUACCAGGAUGACCCUCUGACCAAAGGC
GUGGACCUGAGAAACGCCCAAGUGACCGAACUGCUCCAGCGGCUG
CUGCAUAGAGCUUGA

Hence, preferably the RNA construct comprises a sequence substantially as set out in SEQ ID No: 75, or a fragment or variant thereof.

In one embodiment, the inhibitor of an innate signalling pathway, or a dominant negative form thereof, is STAT2 dominant negative long form, which binds to IRF9. STAT2 (NCBI Reference Sequence: NM_005419.4; UniProtKB—P52630 (STAT2_HUMAN)), or an orthologue thereof, and which may be rendered dominant negative by a F175D Y701F mutation (STAT2 (1-851-F175DY701F)) that can act in a dominant negative fashion to block ISGF-3 formation (Rengachari S, Groiss S, Devos J M, Caron E, Grandvaux N, Panne D. Structure of the STAT2-IRF9 complex. PNAS. 2018, 115 (4) E601-E609; DOI: 10.1073/pnas.1718426115), and is represented herein as SEQ ID No: 76

[SEQ ID No: 76]
MAQWEMLQNLDSPFQDQLHQLYSHSLLPVDIRQYLAVWIEDQNWQ
EAALGSDDSKATMLFFHFLDQLNYECGRCSQDPESLLLQHNLRKF
CRDIQPFSQDPTQLAEMIFNLLLEEKRILIQAQRAQLEQGEPVLE
TPVESQQHEIESRILDLRAMMEKLVKSISQLKDQQDVFCDRYKIQ
AKGKTPSLDPHQTKEQKILQETLNELDKRRKEVLDASKALLGRLT
TLIELLLPKLEEWKAQQQKACIRAPIDHGLEQLETWFTAGAKLLF
HLRQLLKELKGLSCLVSYQDDPLTKGVDLRNAQVTELLQRLLHRA
FVVETQPCMPQTPHRPLILKTGSKFTVRTRLLVRLQEGNESLTVE
VSIDRNPPQLQGFRKFNILTSNQKTLTPEKGQSQGLIWDFGYLTL
VEQRSGGSGKGSNKGPLGVTEELHIISFTVKYTYQGLKQELKTDT
LPVVIISNMNQLSIAWASVLWFNLLSPNLQNQQFFSNPPKAPWSL
LGPALSWQFSSYVGRGLNSDQLSMLRNKLFGQNCRTEDPLLSWAD
FTKRESPPGKLPFWTWLDKILELVHDHLKDLWNDGRIMGFVSRSQ
ERRLLKKTMSGTFLLRFSESSEGGITCSWVEHQDDDKVLIYSVQP
YTKEVLQSLPLTEIIRHYQLLTEENIPENPLRFLYPRIPRDEAFG
CYYQEKVNLQERRKYLKHRLIVVSNRQVDELQQPLELKPEPELES
LELELGLVPEPELSLDLEPLLKAGLDLGPELESVLESTLEPVIEP
TLCMVSQTVPEPDQGPVSQPVPEPDLPCDLRHLNTEPMEIFRNCV
KIEEIMPNGDPLLAGQNTVDEVYVSRPSHFYTDGPLMPSDF

Therefore, preferably the RNA construct of the first aspect comprises a nucleotide sequence which encodes an amino acid sequence substantially as set out in SEQ ID No: 76, or a variant or fragment thereof.

In one embodiment, the STAT2 dominant negative long form polypeptide is encoded by the DNA nucleotide sequence of SEQ ID No: 77, as follows:

[SEQ ID No: 77]
ATGGCGCAGTGGGAAATGCTGCAGAATCTTGACAGCCCCTTTCAG
GATCAGCTGCACCAGCTTTACTCGCACAGCCTCCTGCCTGTGGAC
ATTCGACAGTACTTGGCTGTCTGGATTGAAGACCAGAACTGGCAG
GAAGCTGCACTTGGGAGTGATGATTCCAAGGCTACCATGCTATTC
TTCCACTTCTTGGATCAGCTGAACTATGAGTGTGGCCGTTGCAGC
CAGGACCCAGAGTCCTTGTTGCTGCAGCACAATTTGCGGAAATTC
TGCCGGGACATTCAGCCCTTTTCCCAGGATCCTACCCAGTTGGCT
GAGATGATCTTTAACCTCCTTCTGGAAGAAAAAAGAATTTTGATC
CAGGCTCAGAGGGCCCAATTGGAACAAGGAGAGCCAGTTCTCGAA
ACACCTGTGGAGAGCCAGCAACATGAGATTGAATCCCGGATCCTG
GATTTAAGGGCTATGATGGAGAAGCTGGTAAAATCCATCAGCCAA
CTGAAAGACCAGCAGGATGTCTTCTGCGACCGATATAAGATCCAG
GCCAAAGGGAAGACACCCTCTCTGGACCCCCATCAGACCAAAGAG
CAGAAGATTCTGCAGGAAACTCTCAATGAACTGGACAAAAGGAGA
AAGGAGGTGCTGGATGCCTCCAAAGCACTGCTAGGCCGATTAACT
ACCCTAATCGAGCTACTGCTGCCAAAGTTGGAGGAGTGGAAGGCC
CAGCAGCAAAAAGCCTGCATCAGAGCTCCCATTGACCACGGGTTG
GAACAGCTGGAGACATGGTTCACAGCTGGAGCAAAGCTGTTGTTT
CACCTGAGGCAGCTGCTGAAGGAGCTGAAGGGACTGAGTTGCCTG
GTTAGCTATCAGGATGACCCTCTGACCAAAGGGGTGGACCTACGC
AACGCCCAGGTCACAGAGTTGCTACAGCGTCTGCTCCACAGAGCC
TTTGTGGTAGAAACCCAGCCCTGCATGCCCCAAACTCCCCATCGA
CCCCTCATCCTCAAGACTGGCAGCAAGTTCACCGTCCGAACAAGG
CTGCTGGTGAGACTCCAGGAAGGCAATGAGTCACTGACTGTGGAA
GTCTCCATTGACAGGAATCCTCCTCAATTACAAGGCTTCCGGAAG
TTCAACATTCTGACTTCAAACCAGAAAACTTTGACCCCCGAGAAG
GGGCAGAGTCAGGGTTTGATTTGGGACTTTGGTTACCTGACTCTG
GTGGAGCAACGTTCAGGTGGTTCAGGAAAGGGCAGCAATAAGGGG
CCACTAGGTGTGACAGAGGAACTGCACATCATCAGCTTCACGGTC
AAATATACCTACCAGGGTCTGAAGCAGGAGCTGAAAACGGACACC
CTCCCTGTGGTGATTATTTCCAACATGAACCAGCTCTCAATTGCC
TGGGCTTCAGTTCTCTGGTTCAATTTGCTCAGCCCAAACCTTCAG
AACCAGCAGTTCTTCTCCAACCCCCCCAAGGCCCCCTGGAGCTTG
CTGGGCCCTGCTCTCAGTTGGCAGTTCTCCTCCTATGTTGGCCGA
GGCCTCAACTCAGACCAGCTGAGCATGCTGAGAAACAAGCTGTTC
GGGCAGAACTGTAGGACTGAGGATCCATTATTGTCCTGGGCTGAC
TTCACTAAGCGAGAGAGCCCTCCTGGCAAGTTACCATTCTGGACA
TGGCTGGACAAAATTCTGGAGTTGGTACATGACCACCTGAAGGAT
CTCTGGAATGATGGACGCATCATGGGCTTTGTGAGTCGGAGCCAG
GAGCGCCGGCTGCTGAAGAAGACCATGTCTGGCACCTTTCTACTG
CGCTTCAGTGAATCGTCAGAAGGGGGCATTACCTGCTCCTGGGTG
GAGCACCAGGATGATGACAAGGTGCTCATCTACTCTGTGCAACCG
TACACGAAGGAGGTGCTGCAGTCACTCCCGCTGACTGAAATCATC
CGCCATTACCAGTTGCTCACTGAGGAGAATATACCTGAAAACCCA
CTGCGCTTCCTCTATCCCCGAATCCCCCGGGATGAAGCTTTTGGG
TGCTACTACCAGGAGAAAGTTAATCTCCAGGAACGGAGGAAATAC
CTGAAACACAGGCTCATTGTGGTCTCTAATAGACAGGTGGATGAA
CTGCAACAACCGCTGGAGCTTAAGCCAGAGCCAGAGCTGGAGTCA
TTAGAGCTGGAACTAGGGCTGGTGCCAGAGCCAGAGCTCAGCCTG
GACTTAGAGCCACTGCTGAAGGCAGGGCTGGATCTGGGGCCAGAG
CTAGAGTCTGTGCTGGAGTCCACTCTGGAGCCTGTGATAGAGCCC
ACACTATGCATGGTATCACAAACAGTGCCAGAGCCAGACCAAGGA
CCTGTATCACAGCCAGTGCCAGAGCCAGATTTGCCCTGTGATCTG
AGACATTTGAACACTGAGCCAATGGAAATCTTCAGAAACTGTGTA
AAGATTGAAGAAATCATGCCGAATGGTGACCCACTGTTGGCTGGC
CAGAACACCGTGGATGAGGTTTACGTCTCCCGCCCCAGCCACTTC
TACACTGATGGACCCTTGATGCCTTCTGACTTC

Accordingly, preferably the STAT2 dominant negative long form polypeptide is encoded by the DNA nucleotide sequence substantially as set out in SEQ ID NO: 77, or a variant or fragment thereof.

Thus, the RNA construct may comprise an RNA nucleotide sequence of SEQ ID No: 78, as follows:

[SEQ ID No: 78]
AUGGCGCAGUGGGAAAUGCUGCAGAAUCUUGACAGCCCCUUUCAG
GAUCAGCUGCACCAGCUUUACUCGCACAGCCUCCUGCCUGUGGAC
AUUCGACAGUACUUGGCUGUCUGGAUUGAAGACCAGAACUGGCAG
GAAGCUGCACUUGGGAGUGAUGAUUCCAAGGCUACCAUGCUAUUC
UUCCACUUCUUGGAUCAGCUGAACUAUGAGUGUGGCCGUUGCAGC
CAGGACCCAGAGUCCUUGUUGCUGCAGCACAAUUUGCGGAAAUUC
UGCCGGGACAUUCAGCCCUUUUCCCAGGAUCCUACCCAGUUGGCU
GAGAUGAUCUUUAACCUCCUUCUGGAAGAAAAAAGAAUUUUGAUC
CAGGCUCAGAGGGCCCAAUUGGAACAAGGAGAGCCAGUUCUCGAA
ACACCUGUGGAGAGCCAGCAACAUGAGAUUGAAUCCCGGAUCCUG
GAUUUAAGGGCUAUGAUGGAGAAGCUGGUAAAAUCCAUCAGCCAA
CUGAAAGACCAGCAGGAUGUCUUCUGCGACCGAUAUAAGAUCCAG
GCCAAAGGGAAGACACCCUCUCUGGACCCCCAUCAGACCAAAGAG
CAGAAGAUUCUGCAGGAAACUCUCAAUGAACUGGACAAAAGGAGA
AAGGAGGUGCUGGAUGCCUCCAAAGCACUGCUAGGCCGAUUAACU
ACCCUAAUCGAGCUACUGCUGCCAAAGUUGGAGGAGUGGAAGGCC
CAGCAGCAAAAAGCCUGCAUCAGAGCUCCCAUUGACCACGGGUUG
GAACAGCUGGAGACAUGGUUCACAGCUGGAGCAAAGCUGUUGUUU
CACCUGAGGCAGCUGCUGAAGGAGCUGAAGGGACUGAGUUGCCUG
GUUAGCUAUCAGGAUGACCCUCUGACCAAAGGGGUGGACCUACGC
AACGCCCAGGUCACAGAGUUGCUACAGCGUCUGCUCCACAGAGCC
UUUGUGGUAGAAACCCAGCCCUGCAUGCCCCAAACUCCCCAUCGA
CCCCUCAUCCUCAAGACUGGCAGCAAGUUCACCGUCCGAACAAGG
CUGCUGGUGAGACUCCAGGAAGGCAAUGAGUCACUGACUGUGGAA
GUCUCCAUUGACAGGAAUCCUCCUCAAUUACAAGGCUUCCGGAAG
UUCAACAUUCUGACUUCAAACCAGAAAACUUUGACCCCCGAGAAG
GGGCAGAGUCAGGGUUUGAUUUGGGACUUUGGUUACCUGACUCUG
GUGGAGCAACGUUCAGGUGGUUCAGGAAAGGGCAGCAAUAAGGGG
CCACUAGGUGUGACAGAGGAACUGCACAUCAUCAGCUUCACGGUC
AAAUAUACCUACCAGGGUCUGAAGCAGGAGCUGAAAACGGACACC
CUCCCUGUGGUGAUUAUUUCCAACAUGAACCAGCUCUCAAUUGCC
UGGGCUUCAGUUCUCUGGUUCAAUUUGCUCAGCCCAAACCUUCAG
AACCAGCAGUUCUUCUCCAACCCCCCCAAGGCCCCCUGGAGCUUG
CUGGGCCCUGCUCUCAGUUGGCAGUUCUCCUCCUAUGUUGGCCGA
GGCCUCAACUCAGACCAGCUGAGCAUGCUGAGAAACAAGCUGUUC
GGGCAGAACUGUAGGACUGAGGAUCCAUUAUUGUCCUGGGCUGAC
UUCACUAAGCGAGAGAGCCCUCCUGGCAAGUUACCAUUCUGGACA
UGGCUGGACAAAAUUCUGGAGUUGGUACAUGACCACCUGAAGGAU
CUCUGGAAUGAUGGACGCAUCAUGGGCUUUGUGAGUCGGAGCCAG
GAGCGCCGGCUGCUGAAGAAGACCAUGUCUGGCACCUUUCUACUG
CGCUUCAGUGAAUCGUCAGAAGGGGGCAUUACCUGCUCCUGGGUG
GAGCACCAGGAUGAUGACAAGGUGCUCAUCUACUCUGUGCAACCG
UACACGAAGGAGGUGCUGCAGUCACUCCCGCUGACUGAAAUCAUC
CGCCAUUACCAGUUGCUCACUGAGGAGAAUAUACCUGAAAACCCA
CUGCGCUUCCUCUAUCCCCGAAUCCCCCGGGAUGAAGCUUUUGGG
UGCUACUACCAGGAGAAAGUUAAUCUCCAGGAACGGAGGAAAUAC
CUGAAACACAGGCUCAUUGUGGUCUCUAAUAGACAGGUGGAUGAA
CUGCAACAACCGCUGGAGCUUAAGCCAGAGCCAGAGCUGGAGUCA
UUAGAGCUGGAACUAGGGCUGGUGCCAGAGCCAGAGCUCAGCCUG
GACUUAGAGCCACUGCUGAAGGCAGGGCUGGAUCUGGGGCCAGAG
CUAGAGUCUGUGCUGGAGUCCACUCUGGAGCCUGUGAUAGAGCCC
ACACUAUGCAUGGUAUCACAAACAGUGCCAGAGCCAGACCAAGGA
CCUGUAUCACAGCCAGUGCCAGAGCCAGAUUUGCCCUGUGAUCUG
AGACAUUUGAACACUGAGCCAAUGGAAAUCUUCAGAAACUGUGUA
AAGAUUGAAGAAAUCAUGCCGAAUGGUGACCCACUGUUGGCUGGC
CAGAACACCGUGGAUGAGGUUUACGUCUCCCGCCCCAGCCACUUC
UACACUGAUGGACCCUUGAUGCCUUCUGACUUC

Therefore, preferably the RNA construct comprises an RNA nucleotide sequence substantially as set out in SEQ ID No: 78, or a variant or fragment thereof.

The inventors then subjected the protein sequence of SEQ ID No: 76 to codon optimisation for human expression, and one embodiment of the codon optimised nucleic acid (DNA) sequence that includes that includes a start (ATG) and a stop (TGA) codon is provided herein as SEQ ID No: 79, as follows:

[SEQ ID No: 79]
ATGGCCCAGTGGGAGATGCTGCAGAACCTGGACAGCCCCTTCCAG
GATCAGCTGCACCAGCTGTACTCCCACTCTCTGCTGCCCGTGGAC
ATCAGACAGTATCTGGCCGTGTGGATCGAGGACCAGAACTGGCAA
GAAGCCGCTCTGGGCAGCGACGATAGCAAGGCCACAATGCTGTTC
TTCCACTTCCTGGACCAGCTGAACTACGAGTGCGGCAGATGCAGC
CAGGATCCAGAAAGTCTGCTGCTCCAGCACAACCTGCGGAAGTTC
TGCAGAGACATCCAGCCATTCTCTCAGGACCCCACACAGCTGGCC
GAGATGATCTTCAACCTGCTGCTGGAAGAGAAGCGGATCCTGATT
CAGGCCCAGAGAGCCCAGCTGGAACAGGGCGAACCTGTCCTGGAA
ACCCCTGTGGAATCTCAGCAGCACGAGATCGAGAGCCGGATCCTG
GATCTGCGGGCCATGATGGAAAAGCTGGTCAAGAGCATCAGCCAG
CTGAAGGACCAGCAGGACGTGTTCTGCGACCGGTACAAGATCCAG
GCCAAGGGCAAGACCCCTAGCCTGGATCCTCACCAGACCAAAGAG
CAGAAGATCCTGCAAGAGACACTGAACGAGCTGGACAAGCGGCGG
AAAGAAGTGCTGGACGCCTCTAAAGCTCTGCTGGGCAGACTGACC
ACTCTGATCGAACTGCTGCTGCCCAAGCTGGAAGAATGGAAGGCA
CAGCAGCAGAAGGCCTGCATCAGAGCCCCTATCGATCACGGCCTG
GAACAGCTGGAAACCTGGTTTACAGCCGGCGCTAAGCTGCTGTTC
CACCTGAGACAGCTGCTGAAAGAGCTGAAGGGCCTGAGCTGCCTG
GTGTCCTACCAGGATGACCCTCTGACCAAAGGCGTGGACCTGAGA
AACGCCCAAGTGACCGAACTGCTCCAGAGACTGCTGCACAGAGCC
TTCGTGGTGGAAACCCAGCCTTGCATGCCCCAGACACCTCACAGA
CCCCTGATCCTGAAAACCGGCAGCAAGTTCACCGTGCGGACCAGA
CTGCTCGTGCGACTGCAAGAGGGCAATGAGAGCCTGACCGTGGAA
GTGTCCATCGACAGAAACCCTCCACAGCTGCAGGGCTTCAGAAAG
TTCAACATCCTGACCAGCAACCAGAAAACCCTGACACCTGAGAAG
GGCCAGAGCCAGGGACTGATCTGGGACTTCGGCTACCTGACACTG
GTCGAGCAGAGATCTGGCGGCTCTGGCAAGGGCTCTAACAAGGGA
CCTCTGGGCGTGACCGAGGAACTGCACATCATCAGCTTCACCGTG
AAGTACACCTACCAGGGCCTGAAGCAAGAACTCAAGACCGACACA
CTGCCCGTCGTGATCATCAGCAACATGAACCAGCTGTCTATCGCC
TGGGCCAGCGTGCTGTGGTTCAATCTGCTGAGCCCCAACCTGCAG
AATCAGCAGTTCTTCAGCAACCCTCCTAAGGCTCCTTGGAGCCTG
CTGGGACCTGCTCTGAGCTGGCAGTTTAGCAGCTATGTCGGCAGA
GGCCTGAACAGCGATCAGCTGAGCATGCTGCGGAACAAGCTGTTC
GGCCAGAACTGCAGGACCGAGGATCCACTGCTGAGCTGGGCCGAC
TTCACCAAGAGAGAGAGCCCTCCAGGCAAGCTGCCCTTCTGGACT
TGGCTGGACAAAATCCTGGAACTGGTGCACGACCACCTGAAGGAT
CTGTGGAACGACGGCCGGATCATGGGCTTCGTGTCCAGATCTCAA
GAGCGCAGACTGCTGAAAAAGACAATGAGCGGCACCTTCCTGCTG
CGGTTCAGCGAATCTAGCGAAGGCGGCATCACCTGTAGCTGGGTC
GAACACCAGGACGACGACAAGGTGCTGATCTACAGCGTGCAGCCC
TACACCAAAGAGGTGCTGCAAAGCCTGCCTCTGACCGAGATCATC
CGGCACTACCAGCTGCTCACCGAGGAAAACATCCCCGAGAATCCT
CTGCGGTTTCTGTACCCTCGGATCCCCAGAGATGAGGCCTTTGGC
TGCTACTACCAAGAGAAAGTGAATCTGCAAGAGCGGCGCAAGTAC
CTGAAGCACAGACTGATCGTGGTGTCCAACAGACAGGTGGACGAG
CTGCAGCAGCCACTGGAACTGAAGCCTGAGCCAGAGCTGGAAAGC
CTCGAGCTGGAACTTGGACTGGTGCCCGAGCCTGAACTGTCTCTG
GATCTGGAACCTCTGCTGAAGGCCGGACTGGACCTCGGACCTGAA
CTGGAAAGCGTGCTGGAATCCACACTGGAACCTGTGATCGAGCCC
ACACTGTGCATGGTGTCTCAGACCGTGCCTGAACCAGATCAGGGC
CCAGTGTCTCAGCCTGTTCCTGAGCCTGATCTGCCCTGCGATCTG
AGGCACCTGAACACCGAGCCTATGGAAATCTTCCGGAACTGCGTG
AAGATCGAGGAAATCATGCCCAACGGCGACCCTCTGCTGGCCGGA
CAGAATACCGTGGATGAAGTGTACGTGTCCCGGCCTAGCCACTTC
TACACAGACGGACCTCTGATGCCCAGCGACTTCTGA

Hence, preferably the RNA construct is encoded by a DNA sequence substantially as set out in SEQ ID No: 79, or a fragment or variant thereof.

In an embodiment, the RNA sequence corresponding to the codon optimised DNA sequence of SEQ ID No: 79 that includes a start (AUG) and a stop (UGA) codon is provided herein as SEQ ID No: 80, as follows:

[SEQ ID No: 80]
AUGGCCCAGUGGGAGAUGCUGCAGAACCUGGACAGCCCCUUCCAG
GAUCAGCUGCACCAGCUGUACUCCCACUCUCUGCUGCCCGUGGAC
AUCAGACAGUAUCUGGCCGUGUGGAUCGAGGACCAGAACUGGCAA
GAAGCCGCUCUGGGCAGCGACGAUAGCAAGGCCACAAUGCUGUUC
UUCCACUUCCUGGACCAGCUGAACUACGAGUGCGGCAGAUGCAGC
CAGGAUCCAGAAAGUCUGCUGCUCCAGCACAACCUGCGGAAGUUC
UGCAGAGACAUCCAGCCAUUCUCUCAGGACCCCACACAGCUGGCC
GAGAUGAUCUUCAACCUGCUGCUGGAAGAGAAGCGGAUCCUGAUU
CAGGCCCAGAGAGCCCAGCUGGAACAGGGCGAACCUGUCCUGGAA
ACCCCUGUGGAAUCUCAGCAGCACGAGAUCGAGAGCCGGAUCCUG
GAUCUGCGGGCCAUGAUGGAAAAGCUGGUCAAGAGCAUCAGCCAG
CUGAAGGACCAGCAGGACGUGUUCUGCGACCGGUACAAGAUCCAG
GCCAAGGGCAAGACCCCUAGCCUGGAUCCUCACCAGACCAAAGAG
CAGAAGAUCCUGCAAGAGACACUGAACGAGCUGGACAAGCGGCGG
AAAGAAGUGCUGGACGCCUCUAAAGCUCUGCUGGGCAGACUGACC
ACUCUGAUCGAACUGCUGCUGCCCAAGCUGGAAGAAUGGAAGGCA
CAGCAGCAGAAGGCCUGCAUCAGAGCCCCUAUCGAUCACGGCCUG
GAACAGCUGGAAACCUGGUUUACAGCCGGCGCUAAGCUGCUGUUC
CACCUGAGACAGCUGCUGAAAGAGCUGAAGGGCCUGAGCUGCCUG
GUGUCCUACCAGGAUGACCCUCUGACCAAAGGCGUGGACCUGAGA
AACGCCCAAGUGACCGAACUGCUCCAGAGACUGCUGCACAGAGCC
UUCGUGGUGGAAACCCAGCCUUGCAUGCCCCAGACACCUCACAGA
CCCCUGAUCCUGAAAACCGGCAGCAAGUUCACCGUGCGGACCAGA
CUGCUCGUGCGACUGCAAGAGGGCAAUGAGAGCCUGACCGUGGAA
GUGUCCAUCGACAGAAACCCUCCACAGCUGCAGGGCUUCAGAAAG
UUCAACAUCCUGACCAGCAACCAGAAAACCCUGACACCUGAGAAG
GGCCAGAGCCAGGGACUGAUCUGGGACUUCGGCUACCUGACACUG
GUCGAGCAGAGAUCUGGCGGCUCUGGCAAGGGCUCUAACAAGGGA
CCUCUGGGCGUGACCGAGGAACUGCACAUCAUCAGCUUCACCGUG
AAGUACACCUACCAGGGCCUGAAGCAAGAACUCAAGACCGACACA
CUGCCCGUCGUGAUCAUCAGCAACAUGAACCAGCUGUCUAUCGCC
UGGGCCAGCGUGCUGUGGUUCAAUCUGCUGAGCCCCAACCUGCAG
AAUCAGCAGUUCUUCAGCAACCCUCCUAAGGCUCCUUGGAGCCUG
CUGGGACCUGCUCUGAGCUGGCAGUUUAGCAGCUAUGUCGGCAGA
GGCCUGAACAGCGAUCAGCUGAGCAUGCUGCGGAACAAGCUGUUC
GGCCAGAACUGCAGGACCGAGGAUCCACUGCUGAGCUGGGCCGAC
UUCACCAAGAGAGAGAGCCCUCCAGGCAAGCUGCCCUUCUGGACU
UGGCUGGACAAAAUCCUGGAACUGGUGCACGACCACCUGAAGGAU
CUGUGGAACGACGGCCGGAUCAUGGGCUUCGUGUCCAGAUCUCAA
GAGCGCAGACUGCUGAAAAAGACAAUGAGCGGCACCUUCCUGCUG
CGGUUCAGCGAAUCUAGCGAAGGCGGCAUCACCUGUAGCUGGGUC
GAACACCAGGACGACGACAAGGUGCUGAUCUACAGCGUGCAGCCC
UACACCAAAGAGGUGCUGCAAAGCCUGCCUCUGACCGAGAUCAUC
CGGCACUACCAGCUGCUCACCGAGGAAAACAUCCCCGAGAAUCCU
CUGCGGUUUCUGUACCCUCGGAUCCCCAGAGAUGAGGCCUUUGGC
UGCUACUACCAAGAGAAAGUGAAUCUGCAAGAGCGGCGCAAGUAC
CUGAAGCACAGACUGAUCGUGGUGUCCAACAGACAGGUGGACGAG
CUGCAGCAGCCACUGGAACUGAAGCCUGAGCCAGAGCUGGAAAGC
CUCGAGCUGGAACUUGGACUGGUGCCCGAGCCUGAACUGUCUCUG
GAUCUGGAACCUCUGCUGAAGGCCGGACUGGACCUCGGACCUGAA
CUGGAAAGCGUGCUGGAAUCCACACUGGAACCUGUGAUCGAGCCC
ACACUGUGCAUGGUGUCUCAGACCGUGCCUGAACCAGAUCAGGGC
CCAGUGUCUCAGCCUGUUCCUGAGCCUGAUCUGCCCUGCGAUCUG
AGGCACCUGAACACCGAGCCUAUGGAAAUCUUCCGGAACUGCGUG
AAGAUCGAGGAAAUCAUGCCCAACGGCGACCCUCUGCUGGCCGGA
CAGAAUACCGUGGAUGAAGUGUACGUGUCCCGGCCUAGCCACUUC
UACACAGACGGACCUCUGAUGCCCAGCGACUUCUGA

Hence, preferably the RNA construct comprises a sequence substantially as set out in SEQ ID No: 80, or a fragment or variant thereof.

In one embodiment, the at least one IMP may be a USP18 (NCBI Reference Sequence: NM_017414.4; UniProtKB—Q9UMW8 (UBP18_HUMAN), or an orthologue thereof. USP18 is believed to interact with IFNAR2 and STAT2 to block type I interferon signalling. Basters A, Knobeloch K-P, Fritz G. USP18—a multifunctional component in the interferon response. Bioscience Reports 2018; 38; doi.org/10.1042/BSR20180250. Randall G, Chen L, Panis M, Fischer A K, Lindenbach B D, Sun J, Heathcote J, Rice C M, Edwards A M, McGilyray ID. Silencing of USP18 potentiates the antiviral activity of interferon against hepatitis C virus infection. Gastroenterol 2006; 1331(5): 1584-1591.

One embodiment of the USP18 is represented herein as SEQ ID No: 161, as follows:

[SEQ ID No: 161]
MSKAFGLLRQICQSILAESSQSPADLEEKKEEDSNMKREQPRERP
RAWDYPHGLVGLHNIGQTCCLNSLIQVFVMNVDFTRILKRITVPG
ADEQRRSVPFQMLLLLEKMQDSRQKAVRPLELAYCLQKCNVPLFV
QHDAAQLYLKLWNLIKDQITDVHLVERLQALYTIRVKDSLICVDC
AMESSRNSSMLTLPLSLFDVDSKPLKTLEDALHCFFQPRELSSKS
KCFCENCGKKTRGKQVLKLTHLPQTLTIHLMRFSIRNSQTRKICH
SLYFPQSLDFSQILPMKRESCDAEEQSGGQYELFAVIAHVGMADS
GHYCVYIRNAVDGKWFCFNDSNICLVSWEDIQCTYGNPNYHWQET
AYLLVYMKMEC

Therefore, preferably the RNA construct of the first aspect comprises a nucleotide sequence which encodes an amino acid sequence substantially as set out in SEQ ID No: 161, or a variant or fragment thereof.

In one embodiment, the USP18 polypeptide is encoded by the DNA nucleotide sequence of SEQ ID No: 162, as follows:

[SEQ ID No: 162]
ATGAGCAAGGCGTTTGGGCTCCTGAGGCAAATCTGTCAGTCCATC
CTGGCTGAGTCCTCGCAGTCCCCGGCAGATCTTGAAGAAAAGAAG
GAAGAAGACAGCAACATGAAGAGAGAGCAGCCCAGAGAGCGTCCC
AGGGCCTGGGACTACCCTCATGGCCTGGTTGGTTTACACAACATT
GGACAGACCTGCTGCCTTAACTCCTTGATTCAGGTGTTCGTAATG
AATGTGGACTTCACCAGGATATTGAAGAGGATCACGGTGCCCAGG
GGAGCTGACGAGCAGAGGAGAAGCGTCCCTTTCCAGATGCTTCTG
CTGCTGGAGAAGATGCAGGACAGCCGGCAGAAAGCAGTGCGGCCC
CTGGAGCTGGCCTACTGCCTGCAGAAGTGCAACGTGCCCTTGTTT
GTCCAACATGATGCTGCCCAACTGTACCTCAAACTCTGGAACCTG
ATTAAGGACCAGATCACTGATGTGCACTTGGTGGAGAGACTGCAG
GCCCTGTATACGATCCGGGTGAAGGACTCCTTGATTTGCGTTGAC
TGTGCCATGGAGAGTAGCAGAAACAGCAGCATGCTCACCCTCCCA
CTTTCTCTTTTTGATGTGGACTCAAAGCCCCTGAAGACACTGGAG
GACGCCCTGCACTGCTTCTTCCAGCCCAGGGAGTTATCAAGCAAA
AGCAAGTGCTTCTGTGAGAACTGTGGGAAGAAGACCCGTGGGAAA
CAGGTCTTGAAGCTGACCCATTTGCCCCAGACCCTGACAATCCAC
CTCATGCGATTCTCCATCAGGAATTCACAGACGAGAAAGATCTGC
CACTCCCTGTACTTCCCCCAGAGCTTGGATTTCAGCCAGATCCTT
CCAATGAAGCGAGAGTCTTGTGATGCTGAGGAGCAGTCTGGAGGG
CAGTATGAGCTTTTTGCTGTGATTGCGCACGTGGGAATGGCAGAC
TCCGGTCATTACTGTGTCTACATCCGGAATGCTGTGGATGGAAAA
TGGTTCTGCTTCAATGACTCCAATATTTGCTTGGTGTCCTGGGAA
GACATCCAGTGTACCTACGGAAATCCTAACTACCACTGGCAGGAA
ACTGCATATCTTCTGGTTTACATGAAGATGGAGTGC

Accordingly, preferably the USP18 polypeptide is encoded by the DNA nucleotide sequence substantially as set out in SEQ ID NO: 162, or a variant or fragment thereof.

Thus, the RNA construct may comprise an RNA nucleotide sequence of SEQ ID No: 163, as follows:

[SEQ ID No: 163]
AUGAGCAAGGCGUUUGGGCUCCUGAGGCAAAUCUGUCAGUCCAUC
CUGGCUGAGUCCUCGCAGUCCCCGGCAGAUCUUGAAGAAAAGAAG
GAAGAAGACAGCAACAUGAAGAGAGAGCAGCCCAGAGAGCGUCCC
AGGGCCUGGGACUACCCUCAUGGCCUGGUUGGUUUACACAACAUU
GGACAGACCUGCUGCCUUAACUCCUUGAUUCAGGUGUUCGUAAUG
AAUGUGGACUUCACCAGGAUAUUGAAGAGGAUCACGGUGCCCAGG
GGAGCUGACGAGCAGAGGAGAAGCGUCCCUUUCCAGAUGCUUCUG
CUGCUGGAGAAGAUGCAGGACAGCCGGCAGAAAGCAGUGCGGCCC
CUGGAGCUGGCCUACUGCCUGCAGAAGUGCAACGUGCCCUUGUUU
GUCCAACAUGAUGCUGCCCAACUGUACCUCAAACUCUGGAACCUG
AUUAAGGACCAGAUCACUGAUGUGCACUUGGUGGAGAGACUGCAG
GCCCUGUAUACGAUCCGGGUGAAGGACUCCUUGAUUUGCGUUGAC
UGUGCCAUGGAGAGUAGCAGAAACAGCAGCAUGCUCACCCUCCCA
CUUUCUCUUUUUGAUGUGGACUCAAAGCCCCUGAAGACACUGGAG
GACGCCCUGCACUGCUUCUUCCAGCCCAGGGAGUUAUCAAGCAAA
AGCAAGUGCUUCUGUGAGAACUGUGGGAAGAAGACCCGUGGGAAA
CAGGUCUUGAAGCUGACCCAUUUGCCCCAGACCCUGACAAUCCAC
CUCAUGCGAUUCUCCAUCAGGAAUUCACAGACGAGAAAGAUCUGC
CACUCCCUGUACUUCCCCCAGAGCUUGGAUUUCAGCCAGAUCCUU
CCAAUGAAGCGAGAGUCUUGUGAUGCUGAGGAGCAGUCUGGAGGG
CAGUAUGAGCUUUUUGCUGUGAUUGCGCACGUGGGAAUGGCAGAC
UCCGGUCAUUACUGUGUCUACAUCCGGAAUGCUGUGGAUGGAAAA
UGGUUCUGCUUCAAUGACUCCAAUAUUUGCUUGGUGUCCUGGGAA
GACAUCCAGUGUACCUACGGAAAUCCUAACUACCACUGGCAGGAA
ACUGCAUAUCUUCUGGUUUACAUGAAGAUGGAGUGC

Therefore, preferably the RNA construct comprises an RNA nucleotide sequence substantially as set out in SEQ ID No: 163, or a variant or fragment thereof.

The inventors then subjected the protein sequence of SEQ ID No: 161 to codon optimisation for human expression, and one embodiment of the codon optimised nucleic acid (DNA) sequence that includes a start (ATG) and a stop (TGA) codon is provided herein as SEQ ID No: 164, as follows:

[SEQ ID No: 164]
ATGAGCAAGGCCTTCGGCCTGCTGAGACAGATCTGCCAGTCTATC
CTGGCCGAGAGCAGCCAGTCTCCTGCCGATCTGGAAGAGAAGAAA
GAAGAGGACTCCAACATGAAGCGCGAGCAGCCCAGAGAAAGACCC
AGAGCCTGGGATTATCCTCACGGCCTCGTGGGCCTGCACAATATC
GGCCAGACCTGCTGCCTGAACAGCCTGATCCAGGTGTTCGTGATG
AACGTGGACTTCACCCGGATCCTGAAGCGGATCACAGTGCCTAGA
GGCGCCGACGAGCAGAGAAGATCCGTGCCTTTTCAGATGCTGCTG
CTCCTGGAAAAGATGCAGGACAGCCGGCAGAAGGCCGTCAGACCT
CTGGAACTGGCCTACTGCCTGCAGAAATGCAACGTGCCCCTGTTC
GTGCAGCACGATGCCGCTCAGCTGTACCTGAAGCTGTGGAACCTG
ATCAAGGACCAGATCACCGACGTGCACCTGGTGGAAAGACTGCAG
GCCCTGTACACCATCAGAGTGAAGGACTCCCTGATCTGCGTGGAC
TGCGCCATGGAAAGCAGCCGGAATAGCTCCATGCTGACCCTGCCT
CTGAGCCTGTTCGACGTGGACAGCAAGCCCCTGAAAACCCTGGAA
GATGCCCTGCACTGCTTCTTCCAGCCTAGAGAGCTGAGCAGCAAG
AGCAAGTGCTTCTGCGAGAACTGCGGCAAGAAAACCCGGGGCAAA
CAGGTGCTGAAGCTGACCCATCTGCCTCAGACACTGACCATCCAC
CTGATGCGGTTCAGCATCCGGAACAGCCAGACCAGAAAGATCTGT
CACTCCCTGTACTTCCCTCAGTCTCTGGACTTCAGCCAGATTCTG
CCCATGAAGAGAGAGAGCTGCGACGCCGAAGAACAGTCTGGCGGA
CAGTACGAGCTGTTCGCCGTGATTGCCCACGTTGGCATGGCCGAT
AGCGGCCACTACTGCGTGTACATCAGAAACGCCGTGGACGGCAAG
TGGTTCTGTTTCAACGACAGCAATATCTGCCTGGTGTCCTGGGAA
GATATCCAGTGCACCTACGGCAACCCCAACTACCACTGGCAAGAG
ACAGCCTACCTGCTGGTGTACATGAAGATGGAATGCTGA

Hence, preferably the RNA construct is encoded by a DNA sequence substantially as set out in SEQ ID No: 164, or a fragment or variant thereof.

In an embodiment, the RNA sequence corresponding to the codon optimised DNA sequence of SEQ ID No: 164 that includes a start (AUG) and a stop (UGA) codon is provided herein as SEQ ID No: 165, as follows:

[SEQ ID No: 165]
AUGAGCAAGGCCUUCGGCCUGCUGAGACAGAUCUGCCAGUCUAUC
CUGGCCGAGAGCAGCCAGUCUCCUGCCGAUCUGGAAGAGAAGAAA
GAAGAGGACUCCAACAUGAAGCGCGAGCAGCCCAGAGAAAGACCC
AGAGCCUGGGAUUAUCCUCACGGCCUCGUGGGCCUGCACAAUAUC
GGCCAGACCUGCUGCCUGAACAGCCUGAUCCAGGUGUUCGUGAUG
AACGUGGACUUCACCCGGAUCCUGAAGCGGAUCACAGUGCCUAGA
GGCGCCGACGAGCAGAGAAGAUCCGUGCCUUUUCAGAUGCUGCUG
CUCCUGGAAAAGAUGCAGGACAGCCGGCAGAAGGCCGUCAGACCU
CUGGAACUGGCCUACUGCCUGCAGAAAUGCAACGUGCCCCUGUUC
GUGCAGCACGAUGCCGCUCAGCUGUACCUGAAGCUGUGGAACCUG
AUCAAGGACCAGAUCACCGACGUGCACCUGGUGGAAAGACUGCAG
GCCCUGUACACCAUCAGAGUGAAGGACUCCCUGAUCUGCGUGGAC
UGCGCCAUGGAAAGCAGCCGGAAUAGCUCCAUGCUGACCCUGCCU
CUGAGCCUGUUCGACGUGGACAGCAAGCCCCUGAAAACCCUGGAA
GAUGCCCUGCACUGCUUCUUCCAGCCUAGAGAGCUGAGCAGCAAG
AGCAAGUGCUUCUGCGAGAACUGCGGCAAGAAAACCCGGGGCAAA
CAGGUGCUGAAGCUGACCCAUCUGCCUCAGACACUGACCAUCCAC
CUGAUGCGGUUCAGCAUCCGGAACAGCCAGACCAGAAAGAUCUGU
CACUCCCUGUACUUCCCUCAGUCUCUGGACUUCAGCCAGAUUCUG
CCCAUGAAGAGAGAGAGCUGCGACGCCGAAGAACAGUCUGGCGGA
CAGUACGAGCUGUUCGCCGUGAUUGCCCACGUUGGCAUGGCCGAU
AGCGGCCACUACUGCGUGUACAUCAGAAACGCCGUGGACGGCAAG
UGGUUCUGUUUCAACGACAGCAAUAUCUGCCUGGUGUCCUGGGAA
GAUAUCCAGUGCACCUACGGCAACCCCAACUACCACUGGCAAGAG
ACAGCCUACCUGCUGGUGUACAUGAAGAUGGAAUGCUGA

Hence, preferably the RNA construct comprises a sequence substantially as set out in SEQ ID No: 165, or a fragment or variant thereof.

In one embodiment, the at least one IMP may be a SOCS1 polypeptide (NCBI Reference Sequence: NM_003745.2; UniProtKB—O15524 (SOCS1_HUMAN)), a truncated version or an orthologue thereof. (Shao R X, Zhang L, Hong Z, Goto K, Cheng D, Chen W C, Jilg N, Kumthip K, Fusco D N, Peng L F, Chung R T. SOCS1 abrogates IFN's antiviral effect on hepatitis C virus replication. Antiviral Research, 2012, 97(2):101-107).

One embodiment of SOCS1 is represented herein as SEQ ID No: 151, as follows:

[SEQ ID No: 151]
MVAHNQVAADNAVSTAAEPRRRPEPSSSSSSSPAAPARPRPCPAV
PAPAPGDTHFRTFRSHADYRRITRASALLDACGFYWGPLSVHGAH
ERLRAEPVGTFLVRDSRQRNCFFALSVKMASGPTSIRVHFQAGRF
HLDGSRESFDCLFELLEHYVAAPRRMLGAPLRQRRVRPLQELCRQ
RIVATVGRENLARIPLNPVLRDYLSSFPFQI

Therefore, preferably the RNA construct of the first aspect comprises a nucleotide sequence which encodes an amino acid sequence substantially as set out in SEQ ID No: 151, or a variant or fragment thereof.

In one embodiment, the SOCS1 polypeptide is encoded by the DNA nucleotide sequence of SEQ ID No: 152, as follows:

[SEQ ID No: 152]
ATGGTAGCACACAACCAGGTGGCAGCCGACAATGCAGTCTCCACA
GCAGCAGAGCCCCGACGGCGGCCAGAACCTTCCTCCTCTTCCTCC
TCCTCGCCCGCGGCCCCCGCGCGCCCGCGGCCGTGCCCCGCGGTC
CCGGCCCCGGCCCCCGGCGACACGCACTTCCGCACATTCCGTTCG
CACGCCGATTACCGGCGCATCACGCGCGCCAGCGCGCTCCTGGAC
GCCTGCGGATTCTACTGGGGGCCCCTGAGCGTGCACGGGGCGCAC
GAGCGGCTGCGCGCCGAGCCCGTGGGCACCTTCCTGGTGCGCGAC
AGCCGCCAGCGGAACTGCTTTTTCGCCCTTAGCGTGAAGATGGCC
TCGGGACCCACGAGCATCCGCGTGCACTTTCAGGCCGGCCGCTTT
CACCTGGATGGCAGCCGCGAGAGCTTCGACTGCCTCTTCGAGCTG
CTGGAGCACTACGTGGCGGCGCCGCGCCGCATGCTGGGGGCCCCG
CTGCGCCAGCGCCGCGTGCGGCCGCTGCAGGAGCTGTGCCGCCAG
CGCATCGTGGCCACCGTGGGCCGCGAGAACCTGGCTCGCATCCCC
CTCAACCCCGTCCTCCGCGACTACCTGAGCTCCTTCCCCTTCCAG
ATT

Accordingly, preferably the SOCS1 polypeptide is encoded by the DNA nucleotide sequence substantially as set out in SEQ ID NO: 152, or a variant or fragment thereof.

Thus, the RNA construct may comprise an RNA nucleotide sequence of SEQ ID No: 153, as follows:

[SEQ ID No: 153]
AUGGUAGCACACAACCAGGUGGCAGCCGACAAUGCAGUCUCCACA
GCAGCAGAGCCCCGACGGCGGCCAGAACCUUCCUCCUCUUCCUCC
UCCUCGCCCGCGGCCCCCGCGCGCCCGCGGCCGUGCCCCGCGGUC
CCGGCCCCGGCCCCCGGCGACACGCACUUCCGCACAUUCCGUUCG
CACGCCGAUUACCGGCGCAUCACGCGCGCCAGCGCGCUCCUGGAC
GCCUGCGGAUUCUACUGGGGGCCCCUGAGCGUGCACGGGGCGCAC
GAGCGGCUGCGCGCCGAGCCCGUGGGCACCUUCCUGGUGCGCGAC
AGCCGCCAGCGGAACUGCUUUUUCGCCCUUAGCGUGAAGAUGGCC
UCGGGACCCACGAGCAUCCGCGUGCACUUUCAGGCCGGCCGCUUU
CACCUGGAUGGCAGCCGCGAGAGCUUCGACUGCCUCUUCGAGCUG
CUGGAGCACUACGUGGCGGCGCCGCGCCGCAUGCUGGGGGCCCCG
CUGCGCCAGCGCCGCGUGCGGCCGCUGCAGGAGCUGUGCCGCCAG
CGCAUCGUGGCCACCGUGGGCCGCGAGAACCUGGCUCGCAUCCCC
CUCAACCCCGUCCUCCGCGACUACCUGAGCUCCUUCCCCUUCCAG
AUU

Therefore, preferably the RNA construct comprises an RNA nucleotide sequence substantially as set out in SEQ ID No: 153, or a variant or fragment thereof.

The inventors then subjected the protein sequence of SEQ ID No: 151 to codon optimisation for human expression, and one embodiment of the codon optimised nucleic acid (DNA) sequence that includes a start (ATG) and a stop (TGA) codon is provided herein as SEQ ID No: 154, as follows:

[SEQ ID No: 154]
ATGGTGGCCCATAATCAGGTGGCCGCCGATAACGCCGTGTCTACA
GCTGCCGAACCTAGAAGAAGGCCCGAGCCTAGCAGCAGCAGCTCT
AGTTCTCCTGCCGCTCCTGCCAGACCTAGACCTTGTCCTGCTGTT
CCTGCTCCAGCTCCTGGCGACACCCACTTCAGAACCTTTAGAAGC
CACGCCGACTACCGGCGGATCACAAGAGCATCTGCTCTGCTGGAT
GCCTGCGGCTTTTATTGGGGCCCTCTGTCTGTGCACGGCGCCCAC
GAAAGACTGAGAGCTGAACCTGTGGGCACCTTCCTCGTGCGGGAT
AGCAGACAGCGGAACTGCTTCTTTGCCCTGAGCGTGAAGATGGCC
AGCGGACCCACATCCATCAGAGTGCACTTTCAGGCCGGCAGATTC
CACCTGGATGGCAGCAGAGAGAGCTTCGACTGCCTGTTCGAGCTG
CTGGAACACTACGTGGCCGCTCCTAGAAGGATGCTGGGAGCACCC
CTGAGACAGAGAAGAGTGCGGCCTCTGCAAGAGCTGTGCCGGCAG
AGAATCGTGGCCACAGTGGGCAGAGAGAACCTGGCCAGAATTCCT
CTGAACCCCGTGCTGAGAGACTACCTGAGCAGCTTCCCCTTCCAA
ATCTGA

Hence, preferably the RNA construct is encoded by a DNA sequence substantially as set out in SEQ ID No: 154, or a fragment or variant thereof.

In an embodiment, the RNA sequence corresponding to the codon optimised DNA sequence of SEQ ID No: 154 that includes a start (AUG) and a stop (UGA) codon is provided herein as SEQ ID No: 155, as follows:

[SEQ ID No: 155]
AUGGUGGCCCAUAAUCAGGUGGCCGCCGAUAACGCCGUGUCUACA
GCUGCCGAACCUAGAAGAAGGCCCGAGCCUAGCAGCAGCAGCUCU
AGUUCUCCUGCCGCUCCUGCCAGACCUAGACCUUGUCCUGCUGUU
CCUGCUCCAGCUCCUGGCGACACCCACUUCAGAACCUUUAGAAGC
CACGCCGACUACCGGCGGAUCACAAGAGCAUCUGCUCUGCUGGAU
GCCUGCGGCUUUUAUUGGGGCCCUCUGUCUGUGCACGGCGCCCAC
GAAAGACUGAGAGCUGAACCUGUGGGCACCUUCCUCGUGCGGGAU
AGCAGACAGCGGAACUGCUUCUUUGCCCUGAGCGUGAAGAUGGCC
AGCGGACCCACAUCCAUCAGAGUGCACUUUCAGGCCGGCAGAUUC
CACCUGGAUGGCAGCAGAGAGAGCUUCGACUGCCUGUUCGAGCUG
CUGGAACACUACGUGGCCGCUCCUAGAAGGAUGCUGGGAGCACCC
CUGAGACAGAGAAGAGUGCGGCCUCUGCAAGAGCUGUGCCGGCAG
AGAAUCGUGGCCACAGUGGGCAGAGAGAACCUGGCCAGAAUUCCU
CUGAACCCCGUGCUGAGAGACUACCUGAGCAGCUUCCCCUUCCAA
AUCUGA

Hence, preferably the RNA construct comprises a sequence substantially as set out in SEQ ID No: 155, or a fragment or variant thereof.

In one embodiment, the at least one IMP may be a SOCS3 polypeptide (NCBI Reference Sequence: NM_003955.5; UniProtKB—O14543 (SOCS3_HUMAN), a truncated version or an orthologue thereof. (Akhtar L N, Qin H, Muldowney M T, Yanagisawa L L, Kutsch O, Clements J E, Benveniste E N. Suppressor of cytokine signaling 3 inhibits antiviral IFN-beta signaling to enhance HIV-1 replication in macrophages. J Immunol 2010; 185(4):2393-404). One embodiment of the SOCS3 polypeptide is represented herein as SEQ ID No: 156, as follows:

[SEQ ID No: 156]
MVTHSKFPAAGMSRPLDTSLRLKTFSSKSEYQLVVNAVRKLQESG
FYWSAVTGGEANLLLSAEPAGTFLIRDSSDQRHFFTLSVKTQSGT
KNLRIQCEGGSFSLQSDPRSTQPVPRFDCVLKLVHHYMPPPGAPS
FPSPPTEPSSEVPEQPSAQPLPGSPPRRAYYIYSGGEKIPLVLSR
PLSSNVATLQHLCRKTVNGHLDSYEKVTQLPGPIREFLDQYDAPL

Therefore, preferably the RNA construct of the first aspect comprises a nucleotide sequence which encodes an amino acid sequence substantially as set out in SEQ ID No: 156, or a variant or fragment thereof.

In one embodiment, the SOCS3 polypeptide is encoded by the DNA nucleotide sequence of SEQ ID No: 157, as follows:

[SEQ ID No: 157]
ATGGTCACCCACAGCAAGTTTCCCGCCGCCGGGATGAGCCGCCCC
CTGGACACCAGCCTGCGCCTCAAGACCTTCAGCTCCAAGAGCGAG
TACCAGCTGGTGGTGAACGCAGTGCGCAAGCTGCAGGAGAGCGGC
TTCTACTGGAGCGCAGTGACCGGCGGCGAGGCGAACCTGCTGCTC
AGTGCCGAGCCCGCCGGCACCTTTCTGATCCGCGACAGCTCGGAC
CAGCGCCACTTCTTCACGCTCAGCGTCAAGACCCAGTCTGGGACC
AAGAACCTGCGCATCCAGTGTGAGGGGGGCAGCTTCTCTCTGCAG
AGCGATCCCCGGAGCACGCAGCCCGTGCCCCGCTTCGACTGCGTG
CTCAAGCTGGTGCACCACTACATGCCGCCCCCTGGAGCCCCCTCC
TTCCCCTCGCCACCTACTGAACCCTCCTCCGAGGTGCCCGAGCAG
CCGTCTGCCCAGCCACTCCCTGGGAGTCCCCCCAGAAGAGCCTAT
TACATCTACTCCGGGGGCGAGAAGATCCCCCTGGTGTTGAGCCGG
CCCCTCTCCTCCAACGTGGCCACTCTTCAGCATCTCTGTCGGAAG
ACCGTCAACGGCCACCTGGACTCCTATGAGAAAGTCACCCAGCTG
CCGGGGCCCATTCGGGAGTTCCTGGACCAGTACGATGCCCCGCTT

Accordingly, preferably the SOCS3 polypeptide is encoded by the DNA nucleotide sequence substantially as set out in SEQ ID NO: 157, or a variant or fragment thereof.

Thus, the RNA construct may comprise an RNA nucleotide sequence of SEQ ID No: 158, as follows:

[SEQ ID No: 158]
AUGGUCACCCACAGCAAGUUUCCCGCCGCCGGGAUGAGCCGCCCC
CUGGACACCAGCCUGCGCCUCAAGACCUUCAGCUCCAAGAGCGAG
UACCAGCUGGUGGUGAACGCAGUGCGCAAGCUGCAGGAGAGCGGC
UUCUACUGGAGCGCAGUGACCGGCGGCGAGGCGAACCUGCUGCUC
AGUGCCGAGCCCGCCGGCACCUUUCUGAUCCGCGACAGCUCGGAC
CAGCGCCACUUCUUCACGCUCAGCGUCAAGACCCAGUCUGGGACC
AAGAACCUGCGCAUCCAGUGUGAGGGGGGCAGCUUCUCUCUGCAG
AGCGAUCCCCGGAGCACGCAGCCCGUGCCCCGCUUCGACUGCGUG
CUCAAGCUGGUGCACCACUACAUGCCGCCCCCUGGAGCCCCCUCC
UUCCCCUCGCCACCUACUGAACCCUCCUCCGAGGUGCCCGAGCAG
CCGUCUGCCCAGCCACUCCCUGGGAGUCCCCCCAGAAGAGCCUAU
UACAUCUACUCCGGGGGCGAGAAGAUCCCCCUGGUGUUGAGCCGG
CCCCUCUCCUCCAACGUGGCCACUCUUCAGCAUCUCUGUCGGAAG
ACCGUCAACGGCCACCUGGACUCCUAUGAGAAAGUCACCCAGCUG
CCGGGGCCCAUUCGGGAGUUCCUGGACCAGUACGAUGCCCCGCUU

Therefore, preferably the RNA construct comprises an RNA nucleotide sequence substantially as set out in SEQ ID No: 158, or a variant or fragment thereof.

The inventors then subjected the protein sequence of SEQ ID No: 156 to codon optimisation for human expression, and one embodiment of the codon optimised nucleic acid (DNA) sequence that includes a start (ATG) and a stop (TGA) codon is provided herein as SEQ ID No: 159, as follows:

[SEQ ID No: 159]
ATGGTCACCCACAGCAAGTTTCCAGCCGCCGGAATGAGCAGACCC
CTGGATACAAGCCTGCGGCTGAAAACCTTCAGCAGCAAGAGCGAG
TATCAGCTGGTGGTCAACGCCGTGCGGAAGCTGCAAGAGAGCGGC
TTTTATTGGAGCGCCGTGACAGGCGGAGAGGCCAATCTTCTGCTG
TCTGCCGAACCTGCCGGCACCTTCCTGATCAGAGATAGCAGCGAC
CAGCGGCACTTCTTCACCCTGAGCGTGAAAACCCAGAGCGGCACC
AAGAACCTGCGGATCCAATGTGAAGGCGGCAGCTTCAGCCTGCAG
AGCGACCCTAGATCTACCCAGCCTGTGCCTAGATTCGACTGCGTG
CTGAAGCTCGTGCACCACTACATGCCTCCACCTGGCGCTCCTAGC
TTCCCATCTCCTCCAACAGAGCCTAGCAGCGAGGTGCCAGAACAG
CCTTCTGCTCAACCTCTGCCTGGCAGCCCTCCTAGAAGGGCCTAC
TACATCTATTCTGGCGGCGAGAAGATCCCTCTGGTGCTGTCTAGA
CCCCTGAGCAGCAATGTGGCCACTCTGCAGCACCTGTGCAGAAAG
ACCGTGAACGGCCACCTGGACAGCTACGAGAAAGTGACCCAACTG
CCTGGACCTATCAGAGAGTTCCTGGACCAGTACGACGCCCCTCTT
TGA

Hence, preferably the RNA construct is encoded by a DNA sequence substantially as set out in SEQ ID No: 159, or a fragment or variant thereof.

In an embodiment, the RNA sequence corresponding to the codon optimised DNA sequence of SEQ ID No: 159 that includes a start (AUG) and a stop (UGA) codon is provided herein as SEQ ID No: 160, as follows:

[SEQ ID No: 160]
AUGGUCACCCACAGCAAGUUUCCAGCCGCCGGAAUGAGCAGACCC
CUGGAUACAAGCCUGCGGCUGAAAACCUUCAGCAGCAAGAGCGAG
UAUCAGCUGGUGGUCAACGCCGUGCGGAAGCUGCAAGAGAGCGGC
UUUUAUUGGAGCGCCGUGACAGGCGGAGAGGCCAAUCUUCUGCUG
UCUGCCGAACCUGCCGGCACCUUCCUGAUCAGAGAUAGCAGCGAC
CAGCGGCACUUCUUCACCCUGAGCGUGAAAACCCAGAGCGGCACC
AAGAACCUGCGGAUCCAAUGUGAAGGCGGCAGCUUCAGCCUGCAG
AGCGACCCUAGAUCUACCCAGCCUGUGCCUAGAUUCGACUGCGUG
CUGAAGCUCGUGCACCACUACAUGCCUCCACCUGGCGCUCCUAGC
UUCCCAUCUCCUCCAACAGAGCCUAGCAGCGAGGUGCCAGAACAG
CCUUCUGCUCAACCUCUGCCUGGCAGCCCUCCUAGAAGGGCCUAC
UACAUCUAUUCUGGCGGCGAGAAGAUCCCUCUGGUGCUGUCUAGA
CCCCUGAGCAGCAAUGUGGCCACUCUGCAGCACCUGUGCAGAAAG
ACCGUGAACGGCCACCUGGACAGCUACGAGAAAGUGACCCAACUG
CCUGGACCUAUCAGAGAGUUCCUGGACCAGUACGACGCCCCUCUU
UGA

Hence, preferably the RNA construct comprises a sequence substantially as set out in SEQ ID No: 160, or a fragment or variant thereof.

Category 4: Inhibitors of RNA Recognition Systems

In yet another embodiment, the IMP may be configured to inhibit RNA recognition systems.

Hence, the reduction, ablation or blocking of the innate immune response to RNA is preferably achieved by the IMP reducing or blocking recognition of RNA (preferably, long RNA molecules) by a host cell harbouring the RNA construct of the invention. Long RNA can be understood by the skilled person to mean RNA that is at least 1 kb in length, and which can be either ssRNA or dsRNA. Preferably, therefore, the innate modulatory protein encoded by the RNA construct comprises a mutated or non-functional inhibitor of RNA recognition, or a dominant negative form thereof.

In an embodiment, the inhibitor of RNA recognition, is TRBP dsRNA. TRBP is a RISC-loading complex subunit TARBP2 and inhibits PKR (NCBI Reference Sequence: NM_134323.2; UniProtKB—Q15633 (TRBP2_HUMAN)), or an orthologue thereof (Heyam A, Lagos D, Plevin M. Dissecting the roles of TRBP and PACT in double-stranded RNA recognition and processing of noncoding RNAs. Wiley Interdiscip Rev RNA. 2015 May-June; 6(3):271-89. doi: 10.1002/wrna.1272). One embodiment of the TRBP dsRNA dominant negative form (TARBP2(1-234)) is represented herein as SEQ ID No:111, as follows:

[SEQ ID No: 111]
MSEEEQGSGTTTGCGLPSIEQMLAANPGKTPISLLQEYGTRIGKT
PVYDLLKAEGQAHQPNFTFRVTVGDTSCTGQGPSKKAAKHKAAEV
ALKHLKGGSMLEPALEDSSSFSPLDSSLPEDIPVFTAAAAATPVP
SVVLTRSPPMELQPPVSPQQSECNPVGALQELVVQKGWRLPEYTV
TQESGPAHRKEFTMTCRVERFIEIGSGTSKKLAKRNAAAKMLLRV
HTVPLDARD

Therefore, preferably the RNA construct of the first aspect comprises a nucleotide sequence which encodes an amino acid sequence substantially as set out in SEQ ID No: 111, or a variant or fragment thereof.

In one embodiment, the TRBP dsRNA dominant negative form polypeptide is encoded by the DNA nucleotide sequence of SEQ ID No: 112, as follows:

[SEQ ID No: 112]
ATGAGTGAAGAGGAGCAAGGCTCCGGCACTACCACGGGCTGCGGG
CTGCCTAGTATAGAGCAAATGCTGGCCGCCAACCCAGGCAAGACC
CCGATCAGCCTTCTGCAGGAGTATGGGACCAGAATAGGGAAGACG
CCTGTGTACGACCTTCTCAAAGCCGAGGGCCAAGCCCACCAGCCT
AATTTCACCTTCCGGGTCACCGTTGGCGACACCAGCTGCACTGGT
CAGGGCCCCAGCAAGAAGGCAGCCAAGCACAAGGCAGCTGAGGTG
GCCCTCAAACACCTCAAAGGGGGGAGCATGCTGGAGCCGGCCCTG
GAGGACAGCAGTTCTTTTTCTCCCCTAGACTCTTCACTGCCTGAG
GACATTCCGGTTTTTACTGCTGCAGCAGCTGCTACCCCAGTTCCA
TCTGTAGTCCTAACCAGGAGCCCCCCCATGGAACTGCAGCCCCCT
GTCTCCCCTCAGCAGTCTGAGTGCAACCCCGTTGGTGCTCTGCAG
GAGCTGGTGGTGCAGAAAGGCTGGCGGTTGCCGGAGTACACAGTG
ACCCAGGAGTCTGGGCCAGCCCACCGCAAAGAATTCACCATGACC
TGTCGAGTGGAGCGTTTCATTGAGATTGGGAGTGGCACTTCCAAA
AAATTGGCAAAGCGGAATGCGGCGGCCAAAATGCTGCTTCGAGTG
CACACGGTGCCTCTGGATGCCCGGGAT

Accordingly, preferably the TRBP dsRNA dominant negative form polypeptide is encoded by the DNA nucleotide sequence substantially as set out in SEQ ID NO: 112, or a variant or fragment thereof.

Thus, the RNA construct may comprise an RNA nucleotide sequence of SEQ ID No: 113, as follows:

[SEQ ID No: 113]
AUGAGUGAAGAGGAGCAAGGCUCCGGCACUACCACGGGCUGCGGG
CUGCCUAGUAUAGAGCAAAUGCUGGCCGCCAACCCAGGCAAGACC
CCGAUCAGCCUUCUGCAGGAGUAUGGGACCAGAAUAGGGAAGACG
CCUGUGUACGACCUUCUCAAAGCCGAGGGCCAAGCCCACCAGCCU
AAUUUCACCUUCCGGGUCACCGUUGGCGACACCAGCUGCACUGGU
CAGGGCCCCAGCAAGAAGGCAGCCAAGCACAAGGCAGCUGAGGUG
GCCCUCAAACACCUCAAAGGGGGGAGCAUGCUGGAGCCGGCCCUG
GAGGACAGCAGUUCUUUUUCUCCCCUAGACUCUUCACUGCCUGAG
GACAUUCCGGUUUUUACUGCUGCAGCAGCUGCUACCCCAGUUCCA
UCUGUAGUCCUAACCAGGAGCCCCCCCAUGGAACUGCAGCCCCCU
GUCUCCCCUCAGCAGUCUGAGUGCAACCCCGUUGGUGCUCUGCAG
GAGCUGGUGGUGCAGAAAGGCUGGCGGUUGCCGGAGUACACAGUG
ACCCAGGAGUCUGGGCCAGCCCACCGCAAAGAAUUCACCAUGACC
UGUCGAGUGGAGCGUUUCAUUGAGAUUGGGAGUGGCACUUCCAAA
AAAUUGGCAAAGCGGAAUGCGGCGGCCAAAAUGCUGCUUCGAGUG
CACACGGUGCCUCUGGAUGCCCGGGAU

Therefore, preferably the RNA construct comprises an RNA nucleotide sequence substantially as set out in SEQ ID No: 113, or a variant or fragment thereof.

The inventors then subjected the protein sequence of SEQ ID No: 11 to codon optimisation for human expression, and one embodiment of the codon optimised nucleic acid (DNA) sequence that includes a start (ATG) and a stop (TGA) codon is provided herein as SEQ ID No: 114, as follows:

[SEQ ID No: 114]
ATGAGCGAGGAAGAACAAGGCAGCGGCACCACCACAGGATGTGGCCTGCC
TTCTATCGAGCAGATGCTGGCCGCCAATCCTGGCAAGACACCTATCAGCC
TGCTGCAAGAGTACGGCACCCGGATCGGAAAGACCCCTGTGTACGATCTG
CTGAAGGCCGAAGGCCAGGCTCACCAGCCTAACTTCACCTTCAGAGTGAC
CGTGGGCGACACCAGCTGTACAGGACAGGGCCCTTCTAAGAAGGCCGCCA
AACACAAAGCCGCCGAGGTGGCCCTGAAACACCTGAAAGGCGGCTCCATG
CTGGAACCCGCTCTGGAAGATAGCAGCAGCTTCAGCCCTCTGGACAGCAG
CCTGCCTGAGGACATCCCTGTGTTTACAGCCGCTGCCGCTGCTACACCTG
TGCCATCTGTGGTGCTGACCAGATCTCCTCCAATGGAACTGCAGCCTCCT
GTGTCTCCTCAGCAGAGCGAGTGTAATCCTGTGGGCGCCCTGCAAGAACT
GGTGGTGCAAAAAGGATGGCGGCTGCCCGAGTACACCGTGACACAAGAAT
CTGGCCCCGCTCACCGGAAAGAATTCACCATGACCTGCAGAGTGGAACGG
TTCATCGAGATCGGCTCCGGCACCTCTAAGAAGCTGGCCAAGAGAAACGC
CGCTGCCAAGATGCTGCTGCGGGTGCACACAGTTCCTCTGGACGCCAGAG
ATTGA

Hence, preferably the RNA construct is encoded by a DNA sequence substantially as set out in SEQ ID No: 114, or a fragment or variant thereof.

In an embodiment, the RNA sequence corresponding to the codon optimised DNA sequence of SEQ ID No: 114 that includes a start (AUG) and a stop (UGA) codon is provided herein as SEQ ID No: 115, as follows:

[SEQ ID No: 115]
AUGAGCGAGGAAGAACAAGGCAGCGGCACCACCACAGGAUGUGGCCUGCC
UUCUAUCGAGCAGAUGCUGGCCGCCAAUCCUGGCAAGACACCUAUCAGCC
UGCUGCAAGAGUACGGCACCCGGAUCGGAAAGACCCCUGUGUACGAUCUG
CUGAAGGCCGAAGGCCAGGCUCACCAGCCUAACUUCACCUUCAGAGUGAC
CGUGGGCGACACCAGCUGUACAGGACAGGGCCCUUCUAAGAAGGCCGCCA
AACACAAAGCCGCCGAGGUGGCCCUGAAACACCUGAAAGGCGGCUCCAUG
CUGGAACCCGCUCUGGAAGAUAGCAGCAGCUUCAGCCCUCUGGACAGCAG
CCUGCCUGAGGACAUCCCUGUGUUUACAGCCGCUGCCGCUGCUACACCUG
UGCCAUCUGUGGUGCUGACCAGAUCUCCUCCAAUGGAACUGCAGCCUCCU
GUGUCUCCUCAGCAGAGCGAGUGUAAUCCUGUGGGCGCCCUGCAAGAACU
GGUGGUGCAAAAAGGAUGGCGGCUGCCCGAGUACACCGUGACACAAGAAU
CUGGCCCCGCUCACCGGAAAGAAUUCACCAUGACCUGCAGAGUGGAACGG
UUCAUCGAGAUCGGCUCCGGCACCUCUAAGAAGCUGGCCAAGAGAAACGC
CGCUGCCAAGAUGCUGCUGCGGGUGCACACAGUUCCUCUGGACGCCAGAG
AUUGA

Hence, preferably the RNA construct comprises a sequence substantially as set out in SEQ ID No: 115, or a fragment or variant thereof.

In an embodiment, the inhibitor of RNA recognition, or a dominant negative form thereof, is Zinc finger anti-viral protein (Zinc AVP), i.e. a dominant negative inhibitor (NCBI Reference Sequence: NM_020119.4; UniProtKB—Q7Z2W4 (ZCCHV_HUMAN)), or an orthologue thereof (Karki S, et al. Multiple interferon stimulated genes synergize with the zinc finger antiviral protein to mediate anti-alphavirus activity. PLoS One. 2012; 7(5):e37398. doi: 10.1371/journal.pone.0037398, and Meagher J L, et al. Structure of the zinc-finger antiviral protein in complex with RNA reveals a mechanism for selective targeting of CG-rich viral sequences. Proc Natl Acad Sci USA. 2019 Nov. 26; 116(48):24303-24309. doi: 10.1073/pnas.1913232116.).

One embodiment of the Zinc finger anti-viral protein dominant negative form is Zinc AVP (1-200), represented herein as SEQ ID No:116, as follows:

[SEQ ID No: 116]
MADPEVCCFITKILCAHGGRMALDALLQEIALSEPQLCEVLQVAGPDRFV
VLETGGEAGITRSVVATTRARVCRRKYCQRPCDNLHLCKLNLLGRCNYSQ
SERNLCKYSHEVLSEENFKVLKNHELSGLNKEELAVLLLQSDPFFMPEIC
KSYKGEGRQQICNQQPPCSRLHICDHFTRGNCRFPNCLRSHNLMDRKVLA

Therefore, preferably the RNA construct of the first aspect comprises a nucleotide sequence which encodes an amino acid sequence substantially as set out in SEQ ID No: 116, or a variant or fragment thereof.

In one embodiment, the Zinc finger anti-viral protein dominant negative form polypeptide is encoded by the DNA nucleotide sequence of SEQ ID No: 117, as follows:

[SEQ ID No: 117]
ATGGCGGACCCGGAGGTGTGCTGCTTCATCACCAAAATCCTGTGCGCCCA
CGGGGGCCGCATGGCCCTGGACGCGCTGCTCCAGGAGATCGCGCTGTCTG
AGCCGCAGCTCTGTGAGGTGCTGCAGGTGGCCGGGCCCGACCGCTTTGTG
GTGTTGGAGACCGGCGGCGAGGCCGGGATCACCCGATCGGTGGTGGCCAC
CACTCGAGCCCGGGTCTGCCGTCGCAAGTACTGCCAGAGACCCTGCGATA
ACCTGCATCTCTGCAAACTCAACTTGCTGGGCCGGTGCAACTATTCGCAG
TCCGAGCGGAATTTATGCAAATATTCTCATGAGGTTCTCTCAGAAGAGAA
CTTCAAAGTCCTGAAAAATCACGAACTCTCTGGACTGAACAAAGAGGAAT
TAGCAGTGCTCCTCCTCCAAAGTGATCCTTTTTTTATGCCCGAGATATGC
AAAAGTTATAAGGGAGAGGGTCGGCAGCAGATTTGTAACCAGCAGCCACC
GTGTTCAAGACTCCACATCTGTGACCACTTCACCCGAGGGAACTGTCGTT
TTCCCAACTGCCTCCGGTCCCATAACCTGATGGACAGAAAGGTGCTGGCC

Accordingly, preferably the Zinc finger anti-viral protein dominant negative form polypeptide is encoded by the DNA nucleotide sequence substantially as set out in SEQ ID NO: 117, or a variant or fragment thereof.

Thus, the RNA construct may comprise an RNA nucleotide sequence of SEQ ID No: 118, as follows:

[SEQ ID No: 118]
AUGGCGGACCCGGAGGUGUGCUGCUUCAUCACCAAAAUCCUGUGCGCCCA
CGGGGGCCGCAUGGCCCUGGACGCGCUGCUCCAGGAGAUCGCGCUGUCUG
AGCCGCAGCUCUGUGAGGUGCUGCAGGUGGCCGGGCCCGACCGCUUUGUG
GUGUUGGAGACCGGCGGCGAGGCCGGGAUCACCCGAUCGGUGGUGGCCAC
CACUCGAGCCCGGGUCUGCCGUCGCAAGUACUGCCAGAGACCCUGCGAUA
ACCUGCAUCUCUGCAAACUCAACUUGCUGGGCCGGUGCAACUAUUCGCAG
UCCGAGCGGAAUUUAUGCAAAUAUUCUCAUGAGGUUCUCUCAGAAGAGAA
CUUCAAAGUCCUGAAAAAUCACGAACUCUCUGGACUGAACAAAGAGGAAU
UAGCAGUGCUCCUCCUCCAAAGUGAUCCUUUUUUUAUGCCCGAGAUAUGC
AAAAGUUAUAAGGGAGAGGGUCGGCAGCAGAUUUGUAACCAGCAGCCACC
GUGUUCAAGACUCCACAUCUGUGACCACUUCACCCGAGGGAACUGUCGUU
UUCCCAACUGCCUCCGGUCCCAUAACCUGAUGGACAGAAAGGUGCUGGCC

Therefore, preferably the RNA construct comprises an RNA nucleotide sequence substantially as set out in SEQ ID No: 118, or a variant or fragment thereof.

The inventors then subjected the protein sequence of SEQ ID No: 116 to codon optimisation for human expression, and one embodiment of the codon optimised nucleic acid (DNA) sequence that includes a start (ATG) and a stop (TGA) codon is provided herein as SEQ ID No: 119, as follows:

[SEQ ID No: 119]
ATGGCCGATCCTGAAGTGTGCTGCTTCATCACCAAGATCCTGTGCGCCCA
CGGCGGAAGAATGGCTCTGGATGCTCTGCTGCAAGAGATCGCCCTGTCTG
AGCCTCAGCTGTGCGAAGTGCTGCAAGTGGCCGGACCTGACAGATTCGTG
GTGCTGGAAACAGGCGGAGAGGCCGGCATTACCAGATCCGTGGTGGCTAC
CACAAGAGCCAGAGTGTGCCGGCGGAAGTACTGCCAGAGGCCTTGCGATA
ATCTGCACCTGTGCAAGCTGAACCTGCTGGGCAGATGCAACTACAGCCAG
AGCGAGCGGAATCTGTGCAAGTACTCCCACGAGGTGCTGAGCGAAGAGAA
CTTCAAGGTGCTGAAGAACCACGAGCTGAGCGGCCTGAACAAAGAGGAAC
TGGCCGTTCTGCTGCTGCAGAGCGACCCATTCTTCATGCCCGAGATCTGC
AAGAGCTACAAAGGCGAGGGCAGACAGCAGATCTGTAACCAGCAGCCTCC
ATGCAGCAGACTGCACATCTGCGACCACTTCACCCGGGGCAACTGCAGAT
TCCCCAACTGCCTGAGAAGCCACAACCTGATGGACCGGAAGGTGCTGGCT
TGA

Hence, preferably the RNA construct is encoded by a DNA sequence substantially as set out in SEQ ID No: 119, or a fragment or variant thereof.

In an embodiment, the RNA sequence corresponding to the codon optimised DNA sequence of SEQ ID No: 119 that includes a start (AUG) and a stop (UGA) codon is provided herein as SEQ ID No: 120, as follows:

[SEQ ID No: 120]
AUGGCCGAUCCUGAAGUGUGCUGCUUCAUCACCAAGAUCCUGUGCGCCCA
CGGCGGAAGAAUGGCUCUGGAUGCUCUGCUGCAAGAGAUCGCCCUGUCUG
AGCCUCAGCUGUGCGAAGUGCUGCAAGUGGCCGGACCUGACAGAUUCGUG
GUGCUGGAAACAGGCGGAGAGGCCGGCAUUACCAGAUCCGUGGUGGCUAC
CACAAGAGCCAGAGUGUGCCGGCGGAAGUACUGCCAGAGGCCUUGCGAUA
AUCUGCACCUGUGCAAGCUGAACCUGCUGGGCAGAUGCAACUACAGCCAG
AGCGAGCGGAAUCUGUGCAAGUACUCCCACGAGGUGCUGAGCGAAGAGAA
CUUCAAGGUGCUGAAGAACCACGAGCUGAGCGGCCUGAACAAAGAGGAAC
UGGCCGUUCUGCUGCUGCAGAGCGACCCAUUCUUCAUGCCCGAGAUCUGC
AAGAGCUACAAAGGCGAGGGCAGACAGCAGAUCUGUAACCAGCAGCCUCC
AUGCAGCAGACUGCACAUCUGCGACCACUUCACCCGGGGCAACUGCAGAU
UCCCCAACUGCCUGAGAAGCCACAACCUGAUGGACCGGAAGGUGCUGGCU
UGA

Hence, preferably the RNA construct comprises a sequence substantially as set out in SEQ ID No: 120, or a fragment or variant thereof.

In another embodiment, the inhibitor of RNA recognition, or a dominant negative form thereof, is PKR dsRNA binding domain, which blocks PKR activation and also act as a blocker to NF-kappa B activation (NCBI Reference Sequence: NM_002759.4; UniProtKB—P19525 (E2AK2_HUMAN)), or an orthologue thereof (Bou-Nader C, et al. The search for a PKR code-differential regulation of protein kinase R activity by diverse RNA and protein regulators. RNA. 2019 May; 25(5):539-556. doi:10.1261/rna.070169.118.). One embodiment of the PKR dsRNA binding domain (PKR dsRNA DB (1-170)) is represented herein as SEQ ID No: 121, as follows:

[SEQ ID No: 121]
MAGDLSAGFFMEELNTYRQKQGVVLKYQELPNSGPPHDRRFTFQVIIDGR
EFPEGEGRSKKEAKNAAAKLAVEILNKEKKAVSPLLLTTTNSSEGLSMGN
YIGLINRIAQKKRLTVNYEQCASGVHGPEGFHYKCKMGQKEYSIGTGSTK
QEAKQLAAKLAYLQILSEET

Therefore, preferably the RNA construct of the first aspect comprises a nucleotide sequence which encodes an amino acid sequence substantially as set out in SEQ ID No: 121, or a variant or fragment thereof.

In one embodiment, the PKR dsRNA binding domain polypeptide is encoded by the DNA nucleotide sequence of SEQ ID No: 122, as follows:

[SEQ ID No: 122]
ATGGCTGGTGATCTTTCAGCAGGTTTCTTCATGGAGGAACTTAATACATA
CCGTCAGAAGCAGGGAGTAGTACTTAAATATCAAGAACTGCCTAATTCAG
GACCTCCACATGATAGGAGGTTTACATTTCAAGTTATAATAGATGGAAGA
GAATTTCCAGAAGGTGAAGGTAGATCAAAGAAGGAAGCAAAAAATGCCGC
AGCCAAATTAGCTGTTGAGATACTTAATAAGGAAAAGAAGGCAGTTAGTC
CTTTATTATTGACAACAACGAATTCTTCAGAAGGATTATCCATGGGGAAT
TACATAGGCCTTATCAATAGAATTGCCCAGAAGAAAAGACTAACTGTAAA
TTATGAACAGTGTGCATCGGGGGTGCATGGGCCAGAAGGATTTCATTATA
AATGCAAAATGGGACAGAAAGAATATAGTATTGGTACAGGTTCTACTAAA
CAGGAAGCAAAACAATTGGCCGCTAAACTTGCATATCTTCAGATATTATC
AGAAGAAACC

Accordingly, preferably the PKR dsRNA binding domain form polypeptide is encoded by the DNA nucleotide sequence substantially as set out in SEQ ID NO: 122, or a variant or fragment thereof.

Thus, the RNA construct may comprise an RNA nucleotide sequence of SEQ ID No: 123, as follows:

[SEQ ID No: 123]
AUGGCUGGUGAUCUUUCAGCAGGUUUCUUCAUGGAGGAACUUAAUACAUA
CCGUCAGAAGCAGGGAGUAGUACUUAAAUAUCAAGAACUGCCUAAUUCAG
GACCUCCACAUGAUAGGAGGUUUACAUUUCAAGUUAUAAUAGAUGGAAGA
GAAUUUCCAGAAGGUGAAGGUAGAUCAAAGAAGGAAGCAAAAAAUGCCGC
AGCCAAAUUAGCUGUUGAGAUACUUAAUAAGGAAAAGAAGGCAGUUAGUC
CUUUAUUAUUGACAACAACGAAUUCUUCAGAAGGAUUAUCCAUGGGGAAU
UACAUAGGCCUUAUCAAUAGAAUUGCCCAGAAGAAAAGACUAACUGUAAA
UUAUGAACAGUGUGCAUCGGGGGUGCAUGGGCCAGAAGGAUUUCAUUAUA
AAUGCAAAAUGGGACAGAAAGAAUAUAGUAUUGGUACAGGUUCUACUAAA
CAGGAAGCAAAACAAUUGGCCGCUAAACUUGCAUAUCUUCAGAUAUUAUC
AGAAGAAACC

Therefore, preferably the RNA construct comprises an RNA nucleotide sequence substantially as set out in SEQ ID No: 123, or a variant or fragment thereof.

The inventors then subjected the protein sequence of SEQ ID No: 121 to codon optimisation for human expression, and one embodiment of the codon optimised nucleic acid (DNA) sequence that includes that includes a start (ATG) and a stop (TGA) codon is provided herein as SEQ ID No: 124, as follows:

[SEQ ID No: 124]
ATGGCTGGCGATCTGAGCGCCGGCTTCTTCATGGAAGAACTGAACACCTA
CCGGCAGAAACAGGGCGTCGTGCTGAAGTACCAAGAGCTGCCTAATAGCG
GCCCTCCTCACGACCGGCGGTTCACCTTTCAAGTGATCATCGACGGCAGA
GAGTTCCCCGAAGGCGAGGGCAGATCTAAGAAAGAGGCCAAGAACGCCGC
TGCCAAGCTGGCCGTGGAAATCCTGAACAAAGAGAAGAAGGCCGTTTCTC
CCCTGCTGCTGACCACCACCAATAGCTCTGAGGGCCTGAGCATGGGCAAC
TACATCGGCCTGATCAACCGGATCGCCCAGAAAAAGCGGCTGACCGTGAA
CTACGAGCAGTGTGCCAGCGGAGTGCACGGCCCTGAGGGCTTTCACTACA
AGTGCAAGATGGGCCAGAAAGAGTACAGCATCGGCACCGGCAGCACCAAG
CAAGAAGCCAAACAGCTGGCCGCCAAACTGGCCTACCTGCAGATCCTGAG
CGAGGAAACCTGA

Hence, preferably the RNA construct is encoded by a DNA sequence substantially as set out in SEQ ID No: 124, or a fragment or variant thereof.

In an embodiment, the RNA sequence corresponding to the codon optimised DNA sequence of SEQ ID No: 124 that includes a start (AUG) and a stop (UGA) codon is provided herein as SEQ ID No: 125, as follows:

[SEQ ID No: 125]
AUGGCUGGCGAUCUGAGCGCCGGCUUCUUCAUGGAAGAACUGAACACCUA
CCGGCAGAAACAGGGCGUCGUGCUGAAGUACCAAGAGCUGCCUAAUAGCG
GCCCUCCUCACGACCGGCGGUUCACCUUUCAAGUGAUCAUCGACGGCAGA
GAGUUCCCCGAAGGCGAGGGCAGAUCUAAGAAAGAGGCCAAGAACGCCGC
UGCCAAGCUGGCCGUGGAAAUCCUGAACAAAGAGAAGAAGGCCGUUUCUC
CCCUGCUGCUGACCACCACCAAUAGCUCUGAGGGCCUGAGCAUGGGCAAC
UACAUCGGCCUGAUCAACCGGAUCGCCCAGAAAAAGCGGCUGACCGUGAA
CUACGAGCAGUGUGCCAGCGGAGUGCACGGCCCUGAGGGCUUUCACUACA
AGUGCAAGAUGGGCCAGAAAGAGUACAGCAUCGGCACCGGCAGCACCAAG
CAAGAAGCCAAACAGCUGGCCGCCAAACUGGCCUACCUGCAGAUCCUGAG
CGAGGAAACCUGA

Hence, preferably the RNA construct comprises a sequence substantially as set out in SEQ ID No: 125, or a fragment or variant thereof.

In an embodiment, the inhibitor of RNA recognition is an OAS family member. The human genome harbours four OAS family members, namely OAS1, OAS2, OAS3 and OASL1. OAS1/OASL1, OAS2, and OAS3 are composed of one, two and three OAS units, respectively, and bind long dsRNA. Accordingly, in another embodiment, the inhibitor of RNA recognition, or a dominant negative form thereof, is OAS1, OAS2, OAS3 or OASL1.

However, OAS3 preferentially binds long dsRNA relative to the others, and so is preferred. Thus, in an embodiment, the inhibitor of RNA recognition, or a dominant negative form thereof, is OAS3, and most preferably OAS3 Domain I: containing dsRNA binding domain (NCBI Reference Sequence: NM_006187.4; UniProtKB—Q9Y6K5 (OAS3_HUMAN)), or an orthologue thereof (Donovan J, Whitney G, Rath S, Korennykh A. Structural mechanism of sensing long dsRNA via a noncatalytic domain in human oligoadenylate synthetase 3. Proc Natl Acad Sci USA. 2015 Mar. 31; 112(13):3949-54. doi: 10.1073/pnas.1419409112). One embodiment of OAS3 Domain I is referred to as UniProtKB—Q9Y6K5 (1-343), and is represented herein as SEQ ID No:136, as follows:

[SEQ ID No: 136]
MDLYSTPAAALDREVARRLQPRKEFVEKARRALGALAAALRERGGRLGAA
APRVLKTVKGGSSGRGTALKGGCDSELVIFLDCFKSYVDORARRAEILSE
MRASLESWWONPVPGLRLTFPEQSVPGALQFRLTSVDLEDWMDVSLVPAF
NVLGQAGSGVKPKPQVYSTLLNSGCQGGEHAACFTELRRNFVNIRPAKLK
NLILLVKHWYHQVCLQGLWKETLPPVYALELLTIFAWEQGCKKDAFSLAE
GLRTVLGLIQQHQHLCVFWTVNYGFEDPAVGQFLORQLKRPRPVILDPAD
PTWDLGNGAAWHWDLLAQEAASCYDHPCFLRGMGDPVQSWKGP

Therefore, preferably the RNA construct of the first aspect comprises a nucleotide sequence which encodes an amino acid sequence substantially as set out in SEQ ID No: 136, or a variant or fragment thereof.

In one embodiment, the OAS3 Domain I polypeptide is encoded by the DNA nucleotide sequence of SEQ ID No: 137, as follows:

[SEQ ID No: 137]
ATGGACTTGTACAGCACCCCGGCCGCTGCGCTGGACAGGTTCGTGGCCAG
AAGGCTGCAGCCGCGGAAGGAGTTCGTAGAGAAGGCGCGGCGCGCTCTGG
GCGCCCTGGCCGCTGCCCTGAGGGAGCGCGGGGGCCGCCTCGGTGCTGCT
GCCCCGCGGGTGCTGAAAACTGTCAAGGGAGGCTCCTCGGGCCGGGGCAC
AGCTCTCAAGGGTGGCTGTGATTCTGAACTTGTCATCTTCCTCGACTGCT
TCAAGAGCTATGTGGACCAGAGGGCCCGCCGTGCAGAGATCCTCAGTGAG
ATGCGGGCATCGCTGGAATCCTGGTGGCAGAACCCAGTCCCTGGTCTGAG
ACTCACGTTTCCTGAGCAGAGCGTGCCTGGGGCCCTGCAGTTCCGCCTGA
CATCCGTAGATCTTGAGGACTGGATGGATGTTAGCCTGGTGCCTGCCTTC
AATGTCCTGGGTCAGGCCGGCTCCGGCGTCAAACCCAAGCCACAAGTCTA
CTCTACCCTCCTCAACAGTGGCTGCCAAGGGGGCGAGCATGCGGCCTGCT
TCACAGAGCTGCGGAGGAACTTTGTGAACATTCGCCCAGCCAAGTTGAAG
AACCTAATCTTGCTGGTGAAGCACTGGTACCACCAGGTGTGCCTACAGGG
GTTGTGGAAGGAGACGCTGCCCCCGGTCTATGCCCTGGAATTGCTGACCA
TCTTCGCCTGGGAGCAGGGCTGTAAGAAGGATGCTTTCAGCCTAGCCGAA
GGCCTCCGAACTGTCCTGGGCCTGATCCAACAGCATCAGCACCTGTGTGT
TTTCTGGACTGTCAACTATGGCTTCGAGGACCCTGCAGTTGGGCAGTTCT
TGCAGCGGCAGCTTAAGAGACCCAGGCCTGTGATCCTGGACCCAGCTGAC
CCCACATGGGACCTGGGGAATGGGGCAGCCTGGCACTGGGATTTGCTAGC
CCAGGAGGCAGCATCCTGCTATGACCACCCATGCTTTCTGAGGGGGATGG
GGGACCCAGTGCAGTCTTGGAAGGGGCCG

Accordingly, preferably the OAS3 Domain I polypeptide is encoded by the DNA nucleotide sequence substantially as set out in SEQ ID NO: 137, or a variant or fragment thereof.

Thus, the RNA construct may comprise an RNA nucleotide sequence of SEQ ID No: 138, as follows:

[SEQ ID No: 138]
AUGGACUUGUACAGCACCCCGGCCGCUGCGCUGGACAGGUUCGUGGCCAG
AAGGCUGCAGCCGCGGAAGGAGUUCGUAGAGAAGGCGCGGCGCGCUCUGG
GCGCCCUGGCCGCUGCCCUGAGGGAGCGCGGGGGCCGCCUCGGUGCUGCU
GCCCCGCGGGUGCUGAAAACUGUCAAGGGAGGCUCCUCGGGCCGGGGCAC
AGCUCUCAAGGGUGGCUGUGAUUCUGAACUUGUCAUCUUCCUCGACUGCU
UCAAGAGCUAUGUGGACCAGAGGGCCCGCCGUGCAGAGAUCCUCAGUGAG
AUGCGGGCAUCGCUGGAAUCCUGGUGGCAGAACCCAGUCCCUGGUCUGAG
ACUCACGUUUCCUGAGCAGAGCGUGCCUGGGGCCCUGCAGUUCCGCCUGA
CAUCCGUAGAUCUUGAGGACUGGAUGGAUGUUAGCCUGGUGCCUGCCUUC
AAUGUCCUGGGUCAGGCCGGCUCCGGCGUCAAACCCAAGCCACAAGUCUA
CUCUACCCUCCUCAACAGUGGCUGCCAAGGGGGCGAGCAUGCGGCCUGCU
UCACAGAGCUGCGGAGGAACUUUGUGAACAUUCGCCCAGCCAAGUUGAAG
AACCUAAUCUUGCUGGUGAAGCACUGGUACCACCAGGUGUGCCUACAGGG
GUUGUGGAAGGAGACGCUGCCCCCGGUCUAUGCCCUGGAAUUGCUGACCA
UCUUCGCCUGGGAGCAGGGCUGUAAGAAGGAUGCUUUCAGCCUAGCCGAA
GGCCUCCGAACUGUCCUGGGCCUGAUCCAACAGCAUCAGCACCUGUGUGU
UUUCUGGACUGUCAACUAUGGCUUCGAGGACCCUGCAGUUGGGCAGUUCU
UGCAGCGGCAGCUUAAGAGACCCAGGCCUGUGAUCCUGGACCCAGCUGAC
CCCACAUGGGACCUGGGGAAUGGGGCAGCCUGGCACUGGGAUUUGCUAGC
CCAGGAGGCAGCAUCCUGCUAUGACCACCCAUGCUUUCUGAGGGGGAUGG
GGGACCCAGUGCAGUCUUGGAAGGGGCCG

Therefore, preferably the RNA construct comprises an RNA nucleotide sequence substantially as set out in SEQ ID No: 138, or a variant or fragment thereof.

The inventors then subjected the protein sequence of SEQ ID No: 136 to codon optimisation for human expression, and one embodiment of the codon optimised nucleic acid (DNA) sequence that includes a start (ATG) and a stop (TGA) codon is provided herein as SEQ ID No: 139, as follows:

[SEQ ID No: 139]
ATGGACCTGTACAGCACACCAGCCGCCGCTCTGGATAGATTCGTG
GCTAGACGACTGCAGCCCCGGAAAGAATTCGTGGAAAAGGCTCGG
AGAGCCCTGGGAGCACTTGCTGCTGCTCTGAGAGAAAGAGGCGGC
AGACTTGGAGCCGCTGCTCCCAGAGTGCTGAAAACAGTGAAAGGC
GGCAGCAGCGGCAGAGGCACAGCTCTTAAAGGCGGCTGCGATAGC
GAGCTGGTCATCTTCCTGGACTGCTTCAAGAGCTACGTGGACCAG
AGAGCCAGACGGGCCGAGATCCTGTCTGAGATGAGAGCCAGCCTG
GAAAGCTGGTGGCAGAATCCTGTGCCTGGCCTGAGACTGACATTC
CCCGAACAGTCTGTTCCCGGCGCTCTGCAGTTTAGACTGACCTCC
GTGGACCTGGAAGATTGGATGGATGTGTCCCTGGTGCCTGCCTTC
AATGTGCTGGGACAAGCTGGCTCTGGCGTGAAGCCTAAGCCTCAG
GTGTACTCTACCCTGCTGAACTCCGGCTGTCAAGGCGGAGAACAC
GCCGCCTGTTTTACCGAGCTGCGGCGGAACTTCGTGAACATCAGA
CCCGCCAAGCTGAAGAACCTGATCCTGCTGGTCAAGCACTGGTAT
CACCAAGTGTGCCTGCAAGGCCTGTGGAAAGAAACCCTGCCTCCT
GTGTACGCCCTGGAACTGCTGACCATCTTCGCCTGGGAACAGGGC
TGCAAGAAGGACGCCTTTAGCCTGGCCGAGGGCCTGAGAACAGTT
CTGGGACTGATTCAGCAGCACCAGCACCTGTGCGTGTTCTGGACC
GTGAACTACGGCTTCGAGGATCCTGCCGTGGGCCAGTTTCTGCAG
AGACAGCTGAAGAGGCCCAGACCTGTGATCCTGGATCCTGCAGAC
CCTACATGGGACCTCGGAAATGGCGCTGCCTGGCATTGGGATCTG
CTGGCCCAAGAAGCCGCCAGCTGTTACGATCACCCCTGCTTTCTG
AGAGGCATGGGCGATCCTGTGCAGAGCTGGAAGGGACCTTGA

Hence, preferably the RNA construct is encoded by a DNA sequence substantially as set out in SEQ ID No: 139, or a fragment or variant thereof.

In an embodiment, the RNA sequence corresponding to the codon optimised DNA sequence of SEQ ID No: 139 that includes a start (AUG) and a stop (UGA) codon is provided herein as SEQ ID No: 140, as follows:

[SEQ ID No: 140]
AUGGACCUGUACAGCACACCAGCCGCCGCUCUGGAUAGAUUCGUG
GCUAGACGACUGCAGCCCCGGAAAGAAUUCGUGGAAAAGGCUCGG
AGAGCCCUGGGAGCACUUGCUGCUGCUCUGAGAGAAAGAGGCGGC
AGACUUGGAGCCGCUGCUCCCAGAGUGCUGAAAACAGUGAAAGGC
GGCAGCAGCGGCAGAGGCACAGCUCUUAAAGGCGGCUGCGAUAGC
GAGCUGGUCAUCUUCCUGGACUGCUUCAAGAGCUACGUGGACCAG
AGAGCCAGACGGGCCGAGAUCCUGUCUGAGAUGAGAGCCAGCCUG
GAAAGCUGGUGGCAGAAUCCUGUGCCUGGCCUGAGACUGACAUUC
CCCGAACAGUCUGUUCCCGGCGCUCUGCAGUUUAGACUGACCUCC
GUGGACCUGGAAGAUUGGAUGGAUGUGUCCCUGGUGCCUGCCUUC
AAUGUGCUGGGACAAGCUGGCUCUGGCGUGAAGCCUAAGCCUCAG
GUGUACUCUACCCUGCUGAACUCCGGCUGUCAAGGCGGAGAACAC
GCCGCCUGUUUUACCGAGCUGCGGCGGAACUUCGUGAACAUCAGA
CCCGCCAAGCUGAAGAACCUGAUCCUGCUGGUCAAGCACUGGUAU
CACCAAGUGUGCCUGCAAGGCCUGUGGAAAGAAACCCUGCCUCCU
GUGUACGCCCUGGAACUGCUGACCAUCUUCGCCUGGGAACAGGGC
UGCAAGAAGGACGCCUUUAGCCUGGCCGAGGGCCUGAGAACAGUU
CUGGGACUGAUUCAGCAGCACCAGCACCUGUGCGUGUUCUGGACC
GUGAACUACGGCUUCGAGGAUCCUGCCGUGGGCCAGUUUCUGCAG
AGACAGCUGAAGAGGCCCAGACCUGUGAUCCUGGAUCCUGCAGAC
CCUACAUGGGACCUCGGAAAUGGCGCUGCCUGGCAUUGGGAUCUG
CUGGCCCAAGAAGCCGCCAGCUGUUACGAUCACCCCUGCUUUCUG
AGAGGCAUGGGCGAUCCUGUGCAGAGCUGGAAGGGACCUUGA

Hence, preferably the RNA construct comprises a sequence substantially as set out in SEQ ID No: 140, or a fragment or variant thereof.

In a further embodiment, the inhibitor of RNA recognition, or a dominant negative so form thereof, is RNAse L, or an orthologue thereof. RNAse L does not recognise RNA itself; dsRNA is recognised by OAS which it activates to produce 2′,5′-linked oligoadenylates from ATP. When these bind to RNAse L, it becomes activated to an endoribonuclease that degrades RNA (NCBI Reference Sequence: NM_021133.4; UniProtKB—Q05823 (RN5A_HUMAN)), (Tanaka N, Nakanishi M, Kusakabe Y, Goto Y, Kitade Y, Nakamura K T. Structural basis for recognition of 2′,5′-linked oligoadenylates by human ribonuclease L. EMBO J. 2004 Oct. 13; 23(20):3929-38. doi: 10.1038/sj.emboj.7600420). One embodiment of RNAse L dominant negative is represented herein as SEQ ID No: 131, as follows:

[SEQ ID No: 131]
MESRDHNNPQEGPTSSSGRRAAVEDNHLLIKAVQNEDVDLVQQLL
EGGANVNFQEEEGGWTPLHNAVQMSREDIVELLLRHGADPVLRKK
NGATPFILAAIAGSVKLLKLFLSKGADVNECDFYGFTAFMEAAVY
GKVKALKFLYKRGANVNLRRKTKEDQERLRKGGATALMDAAEKGH
VEVLKILLDEMGADVNACDNMGRNALIHALLSSDDSDVEAITHLL
LDHGADVNVRGERGKTPLILAVEKKHLGLVORLLEQEHIEINDTD
SDGKTALLLAVELKLKKIAELLCKRGASTDCGDLVMTARRNYDHS
LVKVLLSHGAKEDFH

Therefore, preferably the RNA construct of the first aspect comprises a nucleotide sequence which encodes an amino acid sequence substantially as set out in SEQ ID No: 131, or a variant or fragment thereof.

In one embodiment, the RNAse L polypeptide is encoded by the DNA nucleotide sequence of SEQ ID No: 132, as follows:

[SEQ ID No: 132]
ATGGAGAGCAGGGATCATAACAACCCCCAGGAGGGACCCACGTCC
TCCAGCGGTAGAAGGGCTGCAGTGGAAGACAATCACTTGCTGATT
AAAGCTGTTCAAAACGAAGATGTTGACCTGGTCCAGCAATTGCTG
GAAGGTGGAGCCAATGTTAATTTCCAGGAAGAGGAAGGGGGCTGG
ACACCTCTGCATAACGCAGTACAAATGAGCAGGGAGGACATTGTG
GAACTTCTGCTTCGTCATGGTGCTGACCCTGTTCTGAGGAAGAAG
AATGGGGCCACGCCTTTTATCCTCGCAGCGATTGCGGGGAGCGTG
AAGCTGCTGAAACTTTTCCTTTCTAAAGGAGCAGATGTCAATGAG
TGTGATTTTTATGGCTTCACAGCCTTCATGGAAGCCGCTGTGTAT
GGTAAGGTCAAAGCCCTAAAATTCCTTTATAAGAGAGGAGCAAAT
GTGAATTTGAGGCGAAAGACAAAGGAGGATCAAGAGCGGCTGAGG
AAAGGAGGGGCCACAGCTCTCATGGACGCTGCTGAAAAAGGACAC
GTAGAGGTCTTGAAGATTCTCCTTGATGAGATGGGGGCAGATGTA
AACGCCTGTGACAATATGGGCAGAAATGCCTTGATCCATGCTCTC
CTGAGCTCTGACGATAGTGATGTGGAGGCTATTACGCATCTGCTG
CTGGACCATGGGGCTGATGTCAATGTGAGGGGAGAAAGAGGGAAG
ACTCCCCTGATCCTGGCAGTGGAGAAGAAGCACTTGGGTTTGGTG
CAGAGGCTTCTGGAGCAAGAGCACATAGAGATTAATGACACAGAC
AGTGATGGCAAAACAGCACTGCTGCTTGCTGTTGAACTCAAACTG
AAGAAAATCGCCGAGTTGCTGTGCAAACGTGGAGCCAGTACAGAT
TGTGGGGATCTTGTTATGACAGCGAGGCGGAATTATGACCATTCC
CTTGTGAAGGTTCTTCTCTCTCATGGAGCCAAAGAAGATTTTCAC

Accordingly, preferably the RNAse L form polypeptide is encoded by the DNA nucleotide sequence substantially as set out in SEQ ID NO: 132, or a variant or fragment thereof.

Thus, the RNA construct may comprise an RNA nucleotide sequence of SEQ ID No: 133, as follows:

[SEQ ID No: 133]
AUGGAGAGCAGGGAUCAUAACAACCCCCAGGAGGGACCCACGUCC
UCCAGCGGUAGAAGGGCUGCAGUGGAAGACAAUCACUUGCUGAUU
AAAGCUGUUCAAAACGAAGAUGUUGACCUGGUCCAGCAAUUGCUG
GAAGGUGGAGCCAAUGUUAAUUUCCAGGAAGAGGAAGGGGGCUGG
ACACCUCUGCAUAACGCAGUACAAAUGAGCAGGGAGGACAUUGUG
GAACUUCUGCUUCGUCAUGGUGCUGACCCUGUUCUGAGGAAGAAG
AAUGGGGCCACGCCUUUUAUCCUCGCAGCGAUUGCGGGGAGCGUG
AAGCUGCUGAAACUUUUCCUUUCUAAAGGAGCAGAUGUCAAUGAG
UGUGAUUUUUAUGGCUUCACAGCCUUCAUGGAAGCCGCUGUGUAU
GGUAAGGUCAAAGCCCUAAAAUUCCUUUAUAAGAGAGGAGCAAAU
GUGAAUUUGAGGCGAAAGACAAAGGAGGAUCAAGAGCGGCUGAGG
AAAGGAGGGGCCACAGCUCUCAUGGACGCUGCUGAAAAAGGACAC
GUAGAGGUCUUGAAGAUUCUCCUUGAUGAGAUGGGGGCAGAUGUA
AACGCCUGUGACAAUAUGGGCAGAAAUGCCUUGAUCCAUGCUCUC
CUGAGCUCUGACGAUAGUGAUGUGGAGGCUAUUACGCAUCUGCUG
CUGGACCAUGGGGCUGAUGUCAAUGUGAGGGGAGAAAGAGGGAAG
ACUCCCCUGAUCCUGGCAGUGGAGAAGAAGCACUUGGGUUUGGUG
CAGAGGCUUCUGGAGCAAGAGCACAUAGAGAUUAAUGACACAGAC
AGUGAUGGCAAAACAGCACUGCUGCUUGCUGUUGAACUCAAACUG
AAGAAAAUCGCCGAGUUGCUGUGCAAACGUGGAGCCAGUACAGAU
UGUGGGGAUCUUGUUAUGACAGCGAGGCGGAAUUAUGACCAUUCC
CUUGUGAAGGUUCUUCUCUCUCAUGGAGCCAAAGAAGAUUUUCAC

Therefore, preferably the RNA construct comprises an RNA nucleotide sequence substantially as set out in SEQ ID No: 133, or a variant or fragment thereof.

The inventors then subjected the protein sequence of SEQ ID No: 133 to codon optimisation for human expression, and one embodiment of the codon optimised nucleic acid (DNA) sequence that includes a start (ATG) and a stop (TGA) codon is provided herein as SEQ ID No: 134, as follows:

[SEQ ID No: 134]
ATGGAAAGCCGGGACCACAACAACCCTCAAGAGGGCCCTACAAGC
AGCTCTGGTAGAAGGGCCGCTGTGGAAGATAACCATCTGCTGATC
AAGGCCGTGCAGAACGAGGACGTGGACCTGGTGCAACAACTGCTG
GAAGGCGGAGCCAACGTGAACTTCCAAGAGGAAGAAGGCGGCTGG
ACCCCTCTGCATAACGCTGTGCAGATGAGCAGAGAGGACATCGTC
GAGCTGCTGCTGAGACATGGCGCTGACCCTGTGCTGAGAAAGAAG
AACGGCGCCACACCTTTCATCCTGGCCGCCATTGCCGGAAGCGTG
AAGCTGCTGAAGCTGTTCCTGAGCAAGGGCGCCGATGTGAACGAG
TGCGACTTCTACGGCTTCACCGCCTTCATGGAAGCCGCCGTGTAC
GGCAAAGTGAAGGCCCTGAAGTTCCTGTACAAGAGGGGCGCTAAC
GTGAACCTGCGGAGAAAGACCAAAGAGGACCAAGAGCGGCTGCGG
AAAGGTGGCGCTACAGCTCTTATGGATGCCGCCGAGAAGGGACAC
GTGGAAGTGCTGAAGATCCTGCTGGATGAGATGGGCGCAGACGTG
AACGCCTGCGACAACATGGGAAGAAACGCCCTGATTCACGCCCTG
CTGAGCAGCGACGATAGCGACGTGGAAGCCATCACACATCTGCTG
CTGGATCACGGGGCTGATGTGAATGTGCGGGGCGAGAGAGGAAAG
ACCCCACTGATTCTGGCCGTGGAAAAGAAACACCTGGGCCTCGTG
CAGAGGCTGCTGGAACAAGAGCACATCGAGATCAACGACACCGAC
AGCGACGGCAAGACAGCCCTGCTGCTTGCCGTGGAACTGAAGCTG
AAGAAGATCGCCGAACTGCTGTGCAAGAGAGGCGCCAGCACAGAT
TGTGGCGACCTCGTGATGACCGCCAGACGGAACTACGATCACAGC
CTGGTCAAGGTGCTGCTGTCCCATGGCGCTAAAGAGGACTTCCAC
TGA

Hence, preferably the RNA construct is encoded by a DNA sequence substantially as set out in SEQ ID No: 134, or a fragment or variant thereof.

In an embodiment, the RNA sequence corresponding to the codon optimised DNA sequence of SEQ ID No: 134 that includes a start (AUG) and a stop (UGA) codon is provided herein as SEQ ID No: 135, as follows:

[SEQ ID No: 135]
AUGGAGAGCAGGGAUCAUAACAACCCCCAGGAGGGACCCACGUCC
UCCAGCGGUAGAAGGGCUGCAGUGGAAGACAAUCACUUGCUGAUU
AAAGCUGUUCAAAACGAAGAUGUUGACCUGGUCCAGCAAUUGCUG
GAAGGUGGAGCCAAUGUUAAUUUCCAGGAAGAGGAAGGGGGCUGG
ACACCUCUGCAUAACGCAGUACAAAUGAGCAGGGAGGACAUUGUG
GAACUUCUGCUUCGUCAUGGUGCUGACCCUGUUCUGAGGAAGAAG
AAUGGGGCCACGCCUUUUAUCCUCGCAGCGAUUGCGGGGAGCGUG
AAGCUGCUGAAACUUUUCCUUUCUAAAGGAGCAGAUGUCAAUGAG
UGUGAUUUUUAUGGCUUCACAGCCUUCAUGGAAGCCGCUGUGUAU
GGUAAGGUCAAAGCCCUAAAAUUCCUUUAUAAGAGAGGAGCAAAU
GUGAAUUUGAGGCGAAAGACAAAGGAGGAUCAAGAGCGGCUGAGG
AAAGGAGGGGCCACAGCUCUCAUGGACGCUGCUGAAAAAGGACAC
GUAGAGGUCUUGAAGAUUCUCCUUGAUGAGAUGGGGGCAGAUGUA
AACGCCUGUGACAAUAUGGGCAGAAAUGCCUUGAUCCAUGCUCUC
CUGAGCUCUGACGAUAGUGAUGUGGAGGCUAUUACGCAUCUGCUG
CUGGACCAUGGGGCUGAUGUCAAUGUGAGGGGAGAAAGAGGGAAG
ACUCCCCUGAUCCUGGCAGUGGAGAAGAAGCACUUGGGUUUGGUG
CAGAGGCUUCUGGAGCAAGAGCACAUAGAGAUUAAUGACACAGAC
AGUGAUGGCAAAACAGCACUGCUGCUUGCUGUUGAACUCAAACUG
AAGAAAAUCGCCGAGUUGCUGUGCAAACGUGGAGCCAGUACAGAU
UGUGGGGAUCUUGUUAUGACAGCGAGGCGGAAUUAUGACCAUUCC
CUUGUGAAGGUUCUUCUCUCUCAUGGAGCCAAAGAAGAUUUUCAC

Hence, preferably the RNA construct comprises a sequence substantially as set out in SEQ ID No: 135, or a fragment or variant thereof.

In an embodiment, the inhibitor of RNA recognition, or a dominant negative form thereof, is of PACT i.e. a dominant negative form, with dsRNA binding domains (1&2) but deletion of c-terminal (domain 3), which prevents PKR activation (NCBI Reference Sequence: NM_003690.5; UniProtKB—O75569 (PRKRA_HUMAN)), or an orthologue thereof (Heyam A, Lagos D, Plevin M. Dissecting the roles of TRBP and PACT in double-stranded RNA recognition and processing of noncoding RNAs. Wiley Interdiscip Rev RNA. 2015 May-June; 6(3):271-89. doi: 10.1002/wrna.1272). One embodiment of the PACT dominant negative form is referred to as >sp|O075569|PRKRA_HUMAN11-194 Interferon-inducible double-stranded RNA-dependent protein kinase activator A OS═Homo sapiens OX=9606 GN=PRKRA PE=1 SV=1 (PACT PRKRA BD (1-194)), and is represented herein as SEQ ID No: 126, as follows:

[SEQ ID No: 126]
MSQSRHRAEAPPLEREDSGTFSLGKMITAKPGKTPIQVLHEYGMK
TKNIPVYECERSDVQIHVPTFTFRVTVGDITCTGEGTSKKLAKHR
AAEAAINILKANASICFAVPDPLMPDPSKOPKNOLNPIGSLQELA
IHHGWRLPEYTLSQEGGPAHKREYTTICRLESFMETGKGASKKQA
KRNAAEKFLAKFSN

Therefore, preferably the RNA construct of the first aspect comprises a nucleotide sequence which encodes an amino acid sequence substantially as set out in SEQ ID No: 126, or a variant or fragment thereof.

In one embodiment, the PACT dominant negative form polypeptide (PACT PRKRA BD (1-194)) is encoded by the DNA nucleotide sequence of SEQ ID No: 127, as follows:

[SEQ ID No: 127]
ATGTCCCAGAGCAGGCACCGCGCCGAGGCCCCGCCGCTGGAGCGC
GAGGACAGTGGGACCTTCAGTTTGGGGAAGATGATAACAGCTAAG
CCAGGGAAAACACCGATTCAGGTATTACACGAATACGGCATGAAG
ACCAAGAACATCCCAGTTTATGAATGTGAAAGATCTGATGTGCAA
ATACACGTGCCCACTTTCACCTTCAGAGTAACCGTTGGTGACATA
ACCTGCACAGGTGAAGGTACAAGTAAGAAGCTGGCGAAACATAGA
GCTGCAGAGGCTGCCATAAACATTTTGAAAGCCAATGCAAGTATT
TGCTTTGCAGTTCCTGACCCCTTAATGCCTGACCCTTCCAAGCAA
CCAAAGAACCAGCTTAATCCTATTGGTTCATTACAGGAATTGGCT
ATTCATCATGGCTGGAGACTTCCTGAATATACCCTTTCCCAGGAG
GGAGGACCTGCTCATAAGAGAGAATATACTACAATTTGCAGGCTA
GAGTCATTTATGGAAACTGGAAAGGGGGCATCAAAAAAGCAAGCC
AAAAGGAATGCTGCTGAGAAATTTCTTGCCAAATTTAGTAAT

Accordingly, preferably the PACT dominant negative form polypeptide is encoded by the DNA nucleotide sequence substantially as set out in SEQ ID NO: 127, or a variant or fragment thereof.

Thus, the RNA construct may comprise an RNA nucleotide sequence of SEQ ID No: 128, as follows:

[SEQ ID No: 128]
AUGUCCCAGAGCAGGCACCGCGCCGAGGCCCCGCCGCUGGAGCGC
GAGGACAGUGGGACCUUCAGUUUGGGGAAGAUGAUAACAGCUAAG
CCAGGGAAAACACCGAUUCAGGUAUUACACGAAUACGGCAUGAAG
ACCAAGAACAUCCCAGUUUAUGAAUGUGAAAGAUCUGAUGUGCAA
AUACACGUGCCCACUUUCACCUUCAGAGUAACCGUUGGUGACAUA
ACCUGCACAGGUGAAGGUACAAGUAAGAAGCUGGCGAAACAUAGA
GCUGCAGAGGCUGCCAUAAACAUUUUGAAAGCCAAUGCAAGUAUU
UGCUUUGCAGUUCCUGACCCCUUAAUGCCUGACCCUUCCAAGCAA
CCAAAGAACCAGCUUAAUCCUAUUGGUUCAUUACAGGAAUUGGCU
AUUCAUCAUGGCUGGAGACUUCCUGAAUAUACCCUUUCCCAGGAG
GGAGGACCUGCUCAUAAGAGAGAAUAUACUACAAUUUGCAGGCUA
GAGUCAUUUAUGGAAACUGGAAAGGGGGCAUCAAAAAAGCAAGCC
AAAAGGAAUGCUGCUGAGAAAUUUCUUGCCAAAUUUAGUAAU

Therefore, preferably the RNA construct comprises an RNA nucleotide sequence substantially as set out in SEQ ID No: 129, or a variant or fragment thereof.

The inventors then subjected the protein sequence of SEQ ID No: 126 to codon optimisation for human expression, and one embodiment of the codon optimised nucleic acid (DNA) sequence that includes a start (ATG) and a stop (TGA) codon is provided herein as SEQ ID No: 129, as follows:

[SEQ ID No: 129]
ATGGCTGGCGATCTGAGCGCCGGCTTCTTCATGGAAGAACTGAAC
ACCTACCGGCAGAAACAGGGCGTCGTGCTGAAGTACCAAGAGCTG
CCTAATAGCGGCCCTCCTCACGACCGGCGGTTCACCTTTCAAGTG
ATCATCGACGGCAGAGAGTTCCCCGAAGGCGAGGGCAGATCTAAG
AAAGAGGCCAAGAACGCCGCTGCCAAGCTGGCCGTGGAAATCCTG
AACAAAGAGAAGAAGGCCGTTTCTCCCCTGCTGCTGACCACCACC
AATAGCTCTGAGGGCCTGAGCATGGGCAACTACATCGGCCTGATC
AACCGGATCGCCCAGAAAAAGCGGCTGACCGTGAACTACGAGCAG
TGTGCCAGCGGAGTGCACGGCCCTGAGGGCTTTCACTACAAGTGC
AAGATGGGCCAGAAAGAGTACAGCATCGGCACCGGCAGCACCAAG
CAAGAAGCCAAACAGCTGGCCGCCAAACTGGCCTACCTGCAGATC
CTGAGCGAGGAAACCTGA

Hence, preferably the RNA construct is encoded by a DNA sequence substantially as set out in SEQ ID No: 129, or a fragment or variant thereof.

In an embodiment, the RNA sequence corresponding to the codon optimised DNA sequence of SEQ ID No: 129 that includes a start (AUG) and a stop (UGA) codon is provided herein as SEQ ID No: 130, as follows:

[SEQ ID No: 130]
AUGAGCCAGAGCAGACACAGAGCCGAAGCUCCUCCACUGGAAAGA
GAGGACAGCGGCACCUUUAGCCUGGGCAAGAUGAUCACAGCCAAG
CCUGGCAAGACCCCUAUCCAGGUGCUGCACGAGUACGGCAUGAAG
ACCAAGAACAUCCCCGUGUACGAGUGCGAGAGAAGCGACGUGCAG
AUCCACGUGCCAACCUUCACCUUCAGAGUGACCGUGGGCGACAUC
ACCUGUACCGGCGAGGGCACAUCUAAGAAGCUGGCCAAACAUAGA
GCCGCCGAGGCCGCCAUCAAUAUCCUGAAGGCCAAUGCCAGCAUC
UGCUUCGCCGUGCCUGAUCCUCUGAUGCCCGAUCCUAGCAAGCAG
CCCAAGAACCAGCUGAACCCUAUCGGCAGCCUGCAAGAGCUGGCC
AUUCAUCAUGGAUGGCGGCUGCCUGAGUACACCCUGUCUCAAGAA
GGCGGCCCUGCUCACAAGAGAGAGUACACCACCAUCUGCCGGCUG
GAAAGCUUCAUGGAAACAGGCAAGGGCGCCAGCAAGAAACAGGCC
AAGAGAAACGCCGCCGAGAAGUUCCUGGCCAAGUUCAGCAACUGA

Hence, preferably the RNA construct comprises a sequence substantially as set out in SEQ ID No: 130, or a fragment or variant thereof.

In one embodiment, the at least one IMP may be a RIG-1 (DDX 58) RNA binding protein C-terminal domain, or a dominant negative form thereof (NCBI Reference Sequence: NM_014314.4; UniProtKB—O95786 (DDX58_HUMAN)), or an orthologue thereof. >sp|O95786|794-925. One embodiment of the RIG-1 dominant negative form is represented herein as SEQ ID No: 141, as follows:

[SEQ ID No: 141]
(M)QEKPKPVPDKENKKLLCRKCKALACYTADVRVIEECHYTVLG
DAFKECFVSRPHPKPKQFSSFEKRAKIFCARQNCSHDWGIHVKYK
TFEIPVIKIESFVVEDIATGVQTLYSKWKDFHFEKIPFDPAEMSK

Therefore, preferably the RNA construct of the first aspect comprises a nucleotide sequence which encodes an amino acid sequence substantially as set out in SEQ ID No: 141, or a variant or fragment thereof.

In one embodiment, the RIG-1 dominant negative form polypeptide is encoded by the DNA nucleotide sequence of SEQ ID No: 142, as follows:

[SEQ ID No: 142]
ATGCAAGAAAAACCAAAACCTGTACCTGATAAGGAAAATAAAAAA
CTGCTCTGCAGAAAGTGCAAAGCCTTGGCATGTTACACAGCTGAC
GTAAGAGTGATAGAGGAATGCCATTACACTGTGCTTGGAGATGCT
TTTAAGGAATGCTTTGTGAGTAGACCACATCCCAAGCCAAAGCAG
TTTTCAAGTTTTGAAAAAAGAGCAAAGATATTCTGTGCCCGACAG
AACTGCAGCCATGACTGGGGAATCCATGTGAAGTACAAGACATTT
GAGATTCCAGTTATAAAAATTGAAAGTTTTGTGGTGGAGGATATT
GCAACTGGAGTTCAGACACTGTACTCGAAGTGGAAGGACTTTCAT
TTTGAGAAGATACCATTTGATCCAGCAGAAATGTCCAAA

Accordingly, preferably the RIG-1 dominant negative form polypeptide is encoded by the DNA nucleotide sequence substantially as set out in SEQ ID NO: 142, or a variant or fragment thereof.

Thus, the RNA construct may comprise an RNA nucleotide sequence of SEQ ID No: 143, as follows:

[SEQ ID No: 143]
AUGCAAGAAAAACCAAAACCUGUACCUGAUAAGGAAAAUAAAAAA
CUGCUCUGCAGAAAGUGCAAAGCCUUGGCAUGUUACACAGCUGAC
GUAAGAGUGAUAGAGGAAUGCCAUUACACUGUGCUUGGAGAUGCU
UUUAAGGAAUGCUUUGUGAGUAGACCACAUCCCAAGCCAAAGCAG
UUUUCAAGUUUUGAAAAAAGAGCAAAGAUAUUCUGUGCCCGACAG
AACUGCAGCCAUGACUGGGGAAUCCAUGUGAAGUACAAGACAUUU
GAGAUUCCAGUUAUAAAAAUUGAAAGUUUUGUGGUGGAGGAUAUU
GCAACUGGAGUUCAGACACUGUACUCGAAGUGGAAGGACUUUCAU
UUUGAGAAGAUACCAUUUGAUCCAGCAGAAAUGUCCAAA

Therefore, preferably the RNA construct comprises an RNA nucleotide sequence substantially as set out in SEQ ID No: 143, or a variant or fragment thereof.

The inventors then subjected the protein sequence of SEQ ID No: 141 to codon optimisation for human expression, and one embodiment of the codon optimised nucleic acid (DNA) sequence that includes a start (ATG) and a stop (TGA) codon is provided herein as SEQ ID No: 144, as follows:

[SEQ ID No: 144]
ATGCAAGAGAAGCCCAAGCCTGTGCCTGACAAAGAGAACA
AGAAACTGCTGTGCCGGAAGTGCAAGGCCCTGGCCTGTTA
TACAGCCGACGTGCGCGTGATCGAGGAATGCCACTATACA
GTGCTGGGCGACGCCTTCAAAGAATGCTTCGTGTCCCGGC
CTCATCCTAAGCCTAAGCAGTTCAGCAGCTTCGAGAAGCG
GGCCAAGATCTTCTGCGCCAGACAGAACTGCAGCCACGAC
TGGGGAATCCACGTGAAGTACAAGACCTTCGAGATCCCCG
TGATCAAGATCGAGAGCTTCGTGGTGGAAGATATCGCCAC
CGGCGTGCAGACCCTGTACAGCAAGTGGAAGGATTTCCAC
TTTGAGAAGATCCCTTTCGACCCCGCCGAGATGAGCAAGT
GA

Hence, preferably the RNA construct is encoded by a DNA sequence substantially as set out in SEQ ID No: 144, or a fragment or variant thereof.

In an embodiment, the RNA sequence corresponding to the codon optimised DNA sequence of SEQ ID No: 144 that includes a start (AUG) and a stop (UGA) codon is provided herein as SEQ ID No: 145, as follows:

[SEQ ID No: 145]
AUGCAAGAGAAGCCCAAGCCUGUGCCUGACAAAGAGAACA
AGAAACUGCUGUGCCGGAAGUGCAAGGCCCUGGCCUGUUA
UACAGCCGACGUGCGCGUGAUCGAGGAAUGCCACUAUACA
GUGCUGGGCGACGCCUUCAAAGAAUGCUUCGUGUCCCGGC
CUCAUCCUAAGCCUAAGCAGUUCAGCAGCUUCGAGAAGCG
GGCCAAGAUCUUCUGCGCCAGACAGAACUGCAGCCACGAC
UGGGGAAUCCACGUGAAGUACAAGACCUUCGAGAUCCCCG
UGAUCAAGAUCGAGAGCUUCGUGGUGGAAGAUAUCGCCAC
CGGCGUGCAGACCCUGUACAGCAAGUGGAAGGAUUUCCAC
UUUGAGAAGAUCCCUUUCGACCCCGCCGAGAUGAGCAAGU
GA

Hence, preferably the RNA construct comprises a sequence substantially as set out in SEQ ID No: 145, or a fragment or variant thereof.

In one embodiment, the at least one IMP may be a RIG splice variant (DDX58_HUMAN_ISOFORM_2) NCBI Reference Sequence: NM_014314.4; UniProtKB—O95786 (DDX58_HUMAN) AA 36-80 deletion, or an orthologue thereof. One embodiment of the RIG splice variant is represented herein as SEQ ID No: 186, as follows:

[SEQ ID No: 186]
MTTEQRRSLQAFQDYIRKTLDPTYILSYMAPWFREGYSGL
YEAIESWDFKKIEKLEEYRLLLKRLQPEFKTRIIPTDIIS
DLSECLINQECEEILQICSTKGMMAGAEKLVECLLRSDKE
NWPKTLKLALEKERNKFSELWIVEKGIKDVETEDLEDKME
TSDIQIFYQEDPECQNLSENSCPPSEVSDTNLYSPFKPRN
YQLELALPAMKGKNTIICAPTGCGKTFVSLLICEHHLKKF
PQGQKGKVVFFANQIPVYEQQKSVFSKYFERHGYRVTGIS
GATAENVPVEQIVENNDIIILTPQILVNNLKKGTIPSLSI
FTLMIFDECHNTSKQHPYNMIMFNYLDQKLGGSSGPLPQV
IGLTASVGVGDAKNTDEALDYICKLCASLDASVIATVKHN
LEELEQVVYKPQKFFRKVESRISDKFKYIIAQLMRDTESL
AKRICKDLENLSQIQNREFGTQKYEQWIVTVQKACMVFQM
PDKDEESRICKALFLYTSHLRKYNDALIISEHARMKDALD
YLKDFFSNVRAAGFDEIEQDLTQRFEEKLQELESVSRDPS
NENPKLEDLCFILQEEYHLNPETITILFVKTRALVDALKN
WIEGNPKLSFLKPGILTGRGKTNQNTGMTLPAQKCILDAF
KASGDHNILIATSVADEGIDIAQCNLVILYEYVGNVIKMI
QTRGRGRARGSKCFLLTSNAGVIEKEQINMYKEKMMNDSI
LRLQTWDEAVFREKILHIQTHEKFIRDSQEKPKPVPDKEN
KKLLCRKCKALACYTADVRVIEECHYTVLGDAFKECFVSR
PHPKPKQFSSFEKRAKIFCARQNCSHDWGIHVKYKTFEIP
VIKIESFVVEDIATGVQTLYSKWKDFHFEKIPFDPAEMSK

Therefore, preferably the RNA construct of the first aspect comprises a nucleotide sequence which encodes an amino acid sequence substantially as set out in SEQ ID No: 186, or a variant or fragment thereof.

In one embodiment, the RIG splice variant is encoded by the DNA nucleotide sequence of SEQ ID No: 187, as follows:

[SEQ ID No: 187]
ATGACCACCGAGCAGCGACGCAGCCTGCAAGCCTTCCAGG
ATTATATCCGGAAGACCCTGGACCCTACCTACATCCTGAG
CTACATGGCCCCCTGGTTTAGGGAGGGTTATTCTGGACTT
TATGAAGCCATTGAAAGTTGGGATTTCAAAAAAATTGAAA
AGTTGGAGGAGTATAGATTACTTTTAAAACGTTTACAACC
AGAATTTAAAACCAGAATTATCCCAACCGATATCATTTCT
GATCTGTCTGAATGTTTAATTAATCAGGAATGTGAAGAAA
TTCTACAGATTTGCTCTACTAAGGGGATGATGGCAGGTGC
AGAGAAATTGGTGGAATGCCTTCTCAGATCAGACAAGGAA
AACTGGCCCAAAACTTTGAAACTTGCTTTGGAGAAAGAAA
GGAACAAGTTCAGTGAACTGTGGATTGTAGAGAAAGGTAT
AAAAGATGTTGAAACAGAAGATCTTGAGGATAAGATGGAA
ACTTCTGACATACAGATTTTCTACCAAGAAGATCCAGAAT
GCCAGAATCTTAGTGAGAATTCATGTCCACCTTCAGAAGT
GTCTGATACAAACTTGTACAGCCCATTTAAACCAAGAAAT
TACCAATTAGAGCTTGCTTTGCCTGCTATGAAAGGAAAAA
ACACAATAATATGTGCTCCTACAGGTTGTGGAAAAACCTT
TGTTTCACTGCTTATATGTGAACATCATCTTAAAAAATTC
CCACAAGGACAAAAGGGGAAAGTTGTCTTTTTTGCGAATC
AGATCCCAGTGTATGAACAGCAGAAATCTGTATTCTCAAA
ATACTTTGAAAGACATGGGTATAGAGTTACAGGCATTTCT
GGAGCAACAGCTGAGAATGTCCCAGTGGAACAGATTGTTG
AGAACAATGACATCATCATTTTAACTCCACAGATTCTTGT
GAACAACCTTAAAAAGGGAACGATTCCATCACTATCCATC
TTTACTTTGATGATATTTGATGAATGCCACAACACTAGTA
AACAACACCCGTACAATATGATCATGTTTAATTATCTAGA
TCAGAAACTTGGAGGATCTTCAGGCCCACTGCCCCAGGTC
ATTGGGCTGACTGCCTCGGTTGGTGTTGGGGATGCCAAAA
ACACAGATGAAGCCTTGGATTATATCTGCAAGCTGTGTGC
TTCTCTTGATGCGTCAGTGATAGCAACAGTCAAACACAAT
CTGGAGGAACTGGAGCAAGTIGTTTATAAGCCCCAGAAGT
TTTTCAGGAAAGTGGAATCACGGATTAGCGACAAATTTAA
ATACATCATAGCTCAGCTGATGAGGGACACAGAGAGTCTG
GCAAAGAGAATCTGCAAAGACCTCGAAAACTTATCTCAAA
TTCAAAATAGGGAATTTGGAACACAGAAATATGAACAATG
GATTGTTACAGTTCAGAAAGCATGCATGGTGTTCCAGATG
CCAGACAAAGATGAAGAGAGCAGGATTTGTAAAGCCCTGT
TTTTATACACTTCACATTTGCGGAAATATAATGATGCCCT
CATTATCAGTGAGCATGCACGAATGAAAGATGCTCTGGAT
TACTTGAAAGACTTCTTCAGCAATGTCCGAGCAGCAGGAT
TCGATGAGATTGAGCAAGATCTTACTCAGAGATTTGAAGA
AAAGCTGCAGGAACTAGAAAGTGTTTCCAGGGATCCCAGC
AATGAGAATCCTAAACTTGAAGACCTCTGCTTCATCTTAC
AAGAAGAGTACCACTTAAACCCAGAGACAATAACAATTCT
CTTTGTGAAAACCAGAGCACTTGTGGACGCTTTAAAAAAT
TGGATTGAAGGAAATCCTAAACTCAGTTTTCTAAAACCTG
GCATATTGACTGGACGTGGCAAAACAAATCAGAACACAGG
AATGACCCTCCCGGCACAGAAGTGTATATTGGATGCATTC
AAAGCCAGTGGAGATCACAATATTCTGATTGCCACCTCAG
TTGCTGATGAAGGCATTGACATTGCACAGTGCAATCTTGT
CATCCTTTATGAGTATGTGGGCAATGTCATCAAAATGATC
CAAACCAGAGGCAGAGGAAGAGCAAGAGGTAGCAAGTGCT
TCCTTCTGACTAGTAATGCTGGTGTAATTGAAAAAGAACA
AATAAACATGTACAAAGAAAAAATGATGAATGACTCTATT
TTACGCCTTCAGACATGGGACGAAGCAGTATTTAGGGAAA
AGATTCTGCATATACAGACTCATGAAAAATTCATCAGAGA
TAGTCAAGAAAAACCAAAACCTGTACCTGATAAGGAAAAT
AAAAAACTGCTCTGCAGAAAGTGCAAAGCCTTGGCATGTT
ACACAGCTGACGTAAGAGTGATAGAGGAATGCCATTACAC
TGTGCTTGGAGATGCTTTTAAGGAATGCTTTGTGAGTAGA
CCACATCCCAAGCCAAAGCAGTTTTCAAGTTTTGAAAAAA
GAGCAAAGATATTCTGTGCCCGACAGAACTGCAGCCATGA
CTGGGGAATCCATGTGAAGTACAAGACATTTGAGATTCCA
GTTATAAAAATTGAAAGTTTTGTGGTGGAGGATATTGCAA
CTGGAGTTCAGACACTGTACTCGAAGTGGAAGGACTTTCA
TTTTGAGAAGATACCATTTGATCCAGCAGAAATGTCCAAA

Accordingly, preferably the RIG splice variant is encoded by the DNA nucleotide sequence substantially as set out in SEQ ID NO: 187, or a variant or fragment thereof.

Thus, the RNA construct may comprise an RNA nucleotide sequence of SEQ ID No: 188, as follows:

[SEQ ID No: 188]
AUGACCACCGAGCAGCGACGCAGCCUGCAAGCCUUCCAGG
AUUAUAUCCGGAAGACCCUGGACCCUACCUACAUCCUGAG
CUACAUGGCCCCCUGGUUUAGGGAGGGUUAUUCUGGACUU
UAUGAAGCCAUUGAAAGUUGGGAUUUCAAAAAAAUUGAAA
AGUUGGAGGAGUAUAGAUUACUUUUAAAACGUUUACAACC
AGAAUUUAAAACCAGAAUUAUCCCAACCGAUAUCAUUUCU
GAUCUGUCUGAAUGUUUAAUUAAUCAGGAAUGUGAAGAAA
UUCUACAGAUUUGCUCUACUAAGGGGAUGAUGGCAGGUGC
AGAGAAAUUGGUGGAAUGCCUUCUCAGAUCAGACAAGGAA
AACUGGCCCAAAACUUUGAAACUUGCUUUGGAGAAAGAAA
GGAACAAGUUCAGUGAACUGUGGAUUGUAGAGAAAGGUAU
AAAAGAUGUUGAAACAGAAGAUCUUGAGGAUAAGAUGGAA
ACUUCUGACAUACAGAUUUUCUACCAAGAAGAUCCAGAAU
GCCAGAAUCUUAGUGAGAAUUCAUGUCCACCUUCAGAAGU
GUCUGAUACAAACUUGUACAGCCCAUUUAAACCAAGAAAU
UACCAAUUAGAGCUUGCUUUGCCUGCUAUGAAAGGAAAAA
ACACAAUAAUAUGUGCUCCUACAGGUUGUGGAAAAACCUU
UGUUUCACUGCUUAUAUGUGAACAUCAUCUUAAAAAAUUC
CCACAAGGACAAAAGGGGAAAGUUGUCUUUUUUGCGAAUC
AGAUCCCAGUGUAUGAACAGCAGAAAUCUGUAUUCUCAAA
AUACUUUGAAAGACAUGGGUAUAGAGUUACAGGCAUUUCU
GGAGCAACAGCUGAGAAUGUCCCAGUGGAACAGAUUGUUG
AGAACAAUGACAUCAUCAUUUUAACUCCACAGAUUCUUGU
GAACAACCUUAAAAAGGGAACGAUUCCAUCACUAUCCAUC
UUUACUUUGAUGAUAUUUGAUGAAUGCCACAACACUAGUA
AACAACACCCGUACAAUAUGAUCAUGUUUAAUUAUCUAGA
UCAGAAACUUGGAGGAUCUUCAGGCCCACUGCCCCAGGUC
AUUGGGCUGACUGCCUCGGUUGGUGUUGGGGAUGCCAAAA
ACACAGAUGAAGCCUUGGAUUAUAUCUGCAAGCUGUGUGC
UUCUCUUGAUGCGUCAGUGAUAGCAACAGUCAAACACAAU
CUGGAGGAACUGGAGCAAGUUGUUUAUAAGCCCCAGAAGU
UUUUCAGGAAAGUGGAAUCACGGAUUAGCGACAAAUUUAA
AUACAUCAUAGCUCAGCUGAUGAGGGACACAGAGAGUCUG
GCAAAGAGAAUCUGCAAAGACCUCGAAAACUUAUCUCAAA
UUCAAAAUAGGGAAUUUGGAACACAGAAAUAUGAACAAUG
GAUUGUUACAGUUCAGAAAGCAUGCAUGGUGUUCCAGAUG
CCAGACAAAGAUGAAGAGAGCAGGAUUUGUAAAGCCCUGU
UUUUAUACACUUCACAUUUGCGGAAAUAUAAUGAUGCCCU
CAUUAUCAGUGAGCAUGCACGAAUGAAAGAUGCUCUGGAU
UACUUGAAAGACUUCUUCAGCAAUGUCCGAGCAGCAGGAU
UCGAUGAGAUUGAGCAAGAUCUUACUCAGAGAUUUGAAGA
AAAGCUGCAGGAACUAGAAAGUGUUUCCAGGGAUCCCAGC
AAUGAGAAUCCUAAACUUGAAGACCUCUGCUUCAUCUUAC
AAGAAGAGUACCACUUAAACCCAGAGACAAUAACAAUUCU
CUUUGUGAAAACCAGAGCACUUGUGGACGCUUUAAAAAAU
UGGAUUGAAGGAAAUCCUAAACUCAGUUUUCUAAAACCUG
GCAUAUUGACUGGACGUGGCAAAACAAAUCAGAACACAGG
AAUGACCCUCCCGGCACAGAAGUGUAUAUUGGAUGCAUUC
AAAGCCAGUGGAGAUCACAAUAUUCUGAUUGCCACCUCAG
UUGCUGAUGAAGGCAUUGACAUUGCACAGUGCAAUCUUGU
CAUCCUUUAUGAGUAUGUGGGCAAUGUCAUCAAAAUGAUC
CAAACCAGAGGCAGAGGAAGAGCAAGAGGUAGCAAGUGCU
UCCUUCUGACUAGUAAUGCUGGUGUAAUUGAAAAAGAACA
AAUAAACAUGUACAAAGAAAAAAUGAUGAAUGACUCUAUU
UUACGCCUUCAGACAUGGGACGAAGCAGUAUUUAGGGAAA
AGAUUCUGCAUAUACAGACUCAUGAAAAAUUCAUCAGAGA
UAGUCAAGAAAAACCAAAACCUGUACCUGAUAAGGAAAAU
AAAAAACUGCUCUGCAGAAAGUGCAAAGCCUUGGCAUGUU
ACACAGCUGACGUAAGAGUGAUAGAGGAAUGCCAUUACAC
UGUGCUUGGAGAUGCUUUUAAGGAAUGCUUUGUGAGUAGA
CCACAUCCCAAGCCAAAGCAGUUUUCAAGUUUUGAAAAAA
GAGCAAAGAUAUUCUGUGCCCGACAGAACUGCAGCCAUGA
CUGGGGAAUCCAUGUGAAGUACAAGACAUUUGAGAUUCCA
GUUAUAAAAAUUGAAAGUUUUGUGGUGGAGGAUAUUGCAA
CUGGAGUUCAGACACUGUACUCGAAGUGGAAGGACUUUCA
UUUUGAGAAGAUACCAUUUGAUCCAGCAGAAAUGUCCAAA

Therefore, preferably the RNA construct comprises an RNA nucleotide sequence substantially as set out in SEQ ID No: 188, or a variant or fragment thereof.

The inventors then subjected the protein sequence of SEQ ID No: 186 to codon optimisation for human expression, and one embodiment of the codon optimised nucleic acid (DNA) sequence that includes a start (ATG) and a stop (TGA) codon is provided herein as SEQ ID No: 189, as follows:

[SEQ ID No: 189]
ATGACCACCGAGCAGAGAAGATCCCTGCAGGCCTTCCAGG
ACTACATCAGAAAGACACTGGACCCCACCTACATCCTGAG
CTACATGGCCCCATGGTTCAGAGAGGGCTACAGCGGACTG
TACGAGGCCATCGAGAGCTGGGACTTCAAGAAGATCGAGA
AGCTGGAAGAGTACCGGCTGCTGCTGAAGAGACTGCAGCC
CGAGTTCAAGACCCGGATCATCCCCACCGACATCATCAGC
GATCTGAGCGAGTGCCTGATCAATCAAGAGTGCGAGGAAA
TCCTGCAGATCTGTAGCACCAAGGGCATGATGGCTGGCGC
CGAGAAACTGGTGGAATGCCTGCTGAGAAGCGACAAAGAG
AACTGGCCCAAGACACTGAAGCTGGCCCTGGAAAAAGAGC
GGAACAAGTTCAGCGAGCTGTGGATCGTGGAAAAGGGCAT
CAAGGACGTGGAAACCGAGGACCTGGAAGATAAGATGGAA
ACCAGCGACATCCAGATCTTCTACCAAGAGGACCCCGAGT
GCCAGAACCTGAGCGAGAATAGCTGCCCTCCTAGCGAGGT
GTCCGACACCAATCTGTACAGCCCCTTCAAGCCCCGGAAC
TACCAGCTGGAACTTGCCCTGCCTGCCATGAAGGGCAAGA
ACACCATCATCTGTGCCCCAACCGGCTGCGGCAAGACCTT
TGTGTCTCTGCTGATCTGCGAGCACCACCTGAAGAAGTTC
CCTCAGGGCCAGAAAGGCAAGGTGGTGTTTTTCGCCAATC
AGATCCCCGTGTACGAGCAGCAGAAAAGCGTGTTCAGCAA
GTACTTCGAGCGGCACGGCTACAGAGTGACAGGCATTTCT
GGCGCCACCGCCGAGAATGTGCCTGTGGAACAGATTGTGG
AAAACAACGATATCATCATCCTGACGCCTCAGATCCTGGT
CAACAATCTGAAGAAGGGCACAATCCCCAGCCTGAGCATC
TTCACCCTGATGATCTTCGACGAGTGCCACAACACCAGCA
AGCAGCACCCCTACAATATGATCATGTTCAACTACCTGGA
CCAGAAGCTCGGCGGCAGCTCTGGACCTCTGCCTCAAGTG
ATTGGCCTGACAGCCTCTGTCGGAGTGGGCGACGCCAAGA
ATACTGACGAGGCCCTGGATTACATCTGCAAGCTGTGCGC
CAGCCTGGACGCCTCTGTGATTGCCACCGTGAAGCACAAC
CTCGAGGAACTGGAACAGGTGGTGTACAAGCCCCAGAAAT
TCTTTCGGAAGGTGGAAAGCCGGATCAGCGACAAGTTCAA
GTACATCATTGCCCAGCTGATGCGGGACACCGAGAGCCTG
GCTAAGAGAATCTGCAAGGATCTGGAAAACCTGAGCCAGA
TCCAGAACAGAGAGTTCGGCACCCAGAAATACGAGCAGTG
GATTGTGACCGTGCAGAAAGCCTGCATGGTGTTCCAGATG
CCTGACAAGGACGAAGAGAGCCGGATCTGCAAAGCCCTGT
TCCTGTACACCAGCCACCTGAGAAAGTACAACGACGCCCT
GATCATCTCCGAGCACGCCAGAATGAAGGACGCCCTGGAC
TACCTGAAGGACTTCTTCTCCAATGTGCGCGCTGCCGGCT
TCGATGAGATCGAGCAAGATCTGACCCAGCGCTTCGAGGA
AAAGCTGCAAGAGCTGGAAAGCGTGTCCAGAGATCCCAGC
AACGAGAACCCCAAACTGGAAGATCTGTGCTTCATCCTGC
AAGAGGAATACCATCTGAACCCCGAGACAATCACCATCCT
GTTCGTGAAAACAAGAGCCCTGGTGGATGCCCTGAAGAAC
TGGATCGAGGGCAACCCCAAGCTGAGCTTCCTGAAGCCTG
GCATCCTGACCGGCAGAGGCAAGACAAACCAGAACACCGG
CATGACCCTGCCAGCTCAGAAGTGCATCCTGGACGCTTTT
AAGGCCAGCGGCGACCACAACATCCTGATCGCCACATCTG
TGGCCGACGAGGGCATCGATATCGCCCAGTGCAATCTGGT
CATCCTGTACGAGTACGTGGGCAACGTGATCAAGATGATC
CAGACAAGAGGCAGGGGCAGAGCCAGAGGCAGCAAGTGCT
TTCTGCTGACCTCTAATGCCGGCGTGATCGAGAAAGAACA
GATCAACATGTACAAAGAAAAGATGATGAACGACAGCATC
CTGCGGCTGCAGACCTGGGATGAAGCCGTGTTCCGGGAAA
AGATCCTGCACATCCAGACACACGAGAAGTTCATCCGGGA
CAGCCAAGAGAAGCCCAAGCCTGTGCCTGACAAAGAAAAC
AAGAAACTGCTGTGCCGGAAGTGCAAGGCCCTGGCCTGTT
ATACAGCCGACGTGCGAGTGATCGAGGAATGCCACTATAC
CGTGCTCGGCGACGCCTTCAAAGAATGCTTCGTGTCCCGG
CCTCATCCTAAGCCTAAGCAGTTCAGCAGCTTCGAGAAGC
GGGCCAAGATCTTCTGCGCCAGACAGAACTGCAGCCACGA
CTGGGGAATCCACGTGAAGTACAAGACCTTCGAGATCCCG
GTCATCAAGATCGAGTCCTTCGTGGTGGAAGATATCGCCA
CCGGCGTGCAGACCCTGTACAGCAAGTGGAAGGATTTCCA
CTTCGAGAAAATCCCTTTCGACCCCGCCGAGATGAGCAAG
TGA

Hence, preferably the RNA construct is encoded by a DNA sequence substantially as set out in SEQ ID No: 189, or a fragment or variant thereof.

In an embodiment, the RNA sequence corresponding to the codon optimised DNA sequence of SEQ ID No: 189 that includes a start (AUG) and a stop (UGA) codon is provided herein as SEQ ID No: 190, as follows:

[SEQ ID No: 190]
AUGACCACCGAGCAGAGAAGAUCCCUGCAGGCCUUCCAGG
ACUACAUCAGAAAGACACUGGACCCCACCUACAUCCUGAG
CUACAUGGCCCCAUGGUUCAGAGAGGGCUACAGCGGACUG
UACGAGGCCAUCGAGAGCUGGGACUUCAAGAAGAUCGAGA
AGCUGGAAGAGUACCGGCUGCUGCUGAAGAGACUGCAGCC
CGAGUUCAAGACCCGGAUCAUCCCCACCGACAUCAUCAGC
GAUCUGAGCGAGUGCCUGAUCAAUCAAGAGUGCGAGGAAA
UCCUGCAGAUCUGUAGCACCAAGGGCAUGAUGGCUGGCGC
CGAGAAACUGGUGGAAUGCCUGCUGAGAAGCGACAAAGAG
AACUGGCCCAAGACACUGAAGCUGGCCCUGGAAAAAGAGC
GGAACAAGUUCAGCGAGCUGUGGAUCGUGGAAAAGGGCAU
CAAGGACGUGGAAACCGAGGACCUGGAAGAUAAGAUGGAA
ACCAGCGACAUCCAGAUCUUCUACCAAGAGGACCCCGAGU
GCCAGAACCUGAGCGAGAAUAGCUGCCCUCCUAGCGAGGU
GUCCGACACCAAUCUGUACAGCCCCUUCAAGCCCCGGAAC
UACCAGCUGGAACUUGCCCUGCCUGCCAUGAAGGGCAAGA
ACACCAUCAUCUGUGCCCCAACCGGCUGCGGCAAGACCUU
UGUGUCUCUGCUGAUCUGCGAGCACCACCUGAAGAAGUUC
CCUCAGGGCCAGAAAGGCAAGGUGGUGUUUUUCGCCAAUC
AGAUCCCCGUGUACGAGCAGCAGAAAAGCGUGUUCAGCAA
GUACUUCGAGCGGCACGGCUACAGAGUGACAGGCAUUUCU
GGCGCCACCGCCGAGAAUGUGCCUGUGGAACAGAUUGUGG
AAAACAACGAUAUCAUCAUCCUGACGCCUCAGAUCCUGGU
CAACAAUCUGAAGAAGGGCACAAUCCCCAGCCUGAGCAUC
UUCACCCUGAUGAUCUUCGACGAGUGCCACAACACCAGCA
AGCAGCACCCCUACAAUAUGAUCAUGUUCAACUACCUGGA
CCAGAAGCUCGGCGGCAGCUCUGGACCUCUGCCUCAAGUG
AUUGGCCUGACAGCCUCUGUCGGAGUGGGCGACGCCAAGA
AUACUGACGAGGCCCUGGAUUACAUCUGCAAGCUGUGCGC
CAGCCUGGACGCCUCUGUGAUUGCCACCGUGAAGCACAAC
CUCGAGGAACUGGAACAGGUGGUGUACAAGCCCCAGAAAU
UCUUUCGGAAGGUGGAAAGCCGGAUCAGCGACAAGUUCAA
GUACAUCAUUGCCCAGCUGAUGCGGGACACCGAGAGCCUG
GCUAAGAGAAUCUGCAAGGAUCUGGAAAACCUGAGCCAGA
UCCAGAACAGAGAGUUCGGCACCCAGAAAUACGAGCAGUG
GAUUGUGACCGUGCAGAAAGCCUGCAUGGUGUUCCAGAUG
CCUGACAAGGACGAAGAGAGCCGGAUCUGCAAAGCCCUGU
UCCUGUACACCAGCCACCUGAGAAAGUACAACGACGCCCU
GAUCAUCUCCGAGCACGCCAGAAUGAAGGACGCCCUGGAC
UACCUGAAGGACUUCUUCUCCAAUGUGCGCGCUGCCGGCU
UCGAUGAGAUCGAGCAAGAUCUGACCCAGCGCUUCGAGGA
AAAGCUGCAAGAGCUGGAAAGCGUGUCCAGAGAUCCCAGC
AACGAGAACCCCAAACUGGAAGAUCUGUGCUUCAUCCUGC
AAGAGGAAUACCAUCUGAACCCCGAGACAAUCACCAUCCU
GUUCGUGAAAACAAGAGCCCUGGUGGAUGCCCUGAAGAAC
UGGAUCGAGGGCAACCCCAAGCUGAGCUUCCUGAAGCCUG
GCAUCCUGACCGGCAGAGGCAAGACAAACCAGAACACCGG
CAUGACCCUGCCAGCUCAGAAGUGCAUCCUGGACGCUUUU
AAGGCCAGCGGCGACCACAACAUCCUGAUCGCCACAUCUG
UGGCCGACGAGGGCAUCGAUAUCGCCCAGUGCAAUCUGGU
CAUCCUGUACGAGUACGUGGGCAACGUGAUCAAGAUGAUC
CAGACAAGAGGCAGGGGCAGAGCCAGAGGCAGCAAGUGCU
UUCUGCUGACCUCUAAUGCCGGCGUGAUCGAGAAAGAACA
GAUCAACAUGUACAAAGAAAAGAUGAUGAACGACAGCAUC
CUGCGGCUGCAGACCUGGGAUGAAGCCGUGUUCCGGGAAA
AGAUCCUGCACAUCCAGACACACGAGAAGUUCAUCCGGGA
CAGCCAAGAGAAGCCCAAGCCUGUGCCUGACAAAGAAAAC
AAGAAACUGCUGUGCCGGAAGUGCAAGGCCCUGGCCUGUU
AUACAGCCGACGUGCGAGUGAUCGAGGAAUGCCACUAUAC
CGUGCUCGGCGACGCCUUCAAAGAAUGCUUCGUGUCCCGG
CCUCAUCCUAAGCCUAAGCAGUUCAGCAGCUUCGAGAAGC
GGGCCAAGAUCUUCUGCGCCAGACAGAACUGCAGCCACGA
CUGGGGAAUCCACGUGAAGUACAAGACCUUCGAGAUCCCG
GUCAUCAAGAUCGAGUCCUUCGUGGUGGAAGAUAUCGCCA
CCGGCGUGCAGACCCUGUACAGCAAGUGGAAGGAUUUCCA
CUUCGAGAAAAUCCCUUUCGACCCCGCCGAGAUGAGCAAG
UGA

Hence, preferably the RNA construct comprises a sequence substantially as set out in SEQ ID No: 190, or a fragment or variant thereof.

The RNA construct comprises a nucleotide sequence which encodes the at least one therapeutic biomolecule. This is referred to as the gene of interest (GOI) in FIG. 1.

The at least one therapeutic biomolecule may comprise a therapeutic protein. The skilled person would understand that therapeutic protein relates to any protein that has therapeutic application, preferably in human. Exemplary therapeutic biomolecules that can be encoded by the RNA molecule include proteins or peptides derived from pathogens, such as bacteria, viruses, fungi, protozoa/or parasites. The protein or peptide may be an antigen, and therefore one which may stimulate or trigger an immune response in the host. Hence, in the embodiment in which the at least one therapeutic biomolecule is an antigen, the RNA construct of the first aspect may be regarded as a vaccine.

The protein or peptide derived from a virus may be a viral antigen. The viral antigen may be derived from a virus selected from the group consisting of: Orthomyxoviruses; Paramyxoviridae viruses; Metapneumovirus and Morbilliviruses; Pneumoviruses; Paramyxoviruses; Poxviridae; Metapneumoviruses; Morbilliviruses; Picornaviruses; Enteroviruseses; Bunyaviruses; Phlebovirus; Nairovirus; Heparnaviruses; Togaviruses; Alphavirus; Arterivirus; Flaviviruses; Pestiviruses; Hepadnaviruses; Rhabdoviruses; Caliciviridae; Coronaviruses; Retroviruses; Reoviruses; Parvoviruses; Delta hepatitis virus (HDV); Hepatitis E virus (HEV); Human Herpesviruses and Papovaviruses.

The Orthomyxoviruses may be Influenza A, B and C. The Paramyxoviridae virus may be Pneumoviruses (RSV), Paramyxoviruses (PIV). The Metapneumovirus may be Morbilliviruses (e.g., measles). The Pneumovirus may be Respiratory syncytial virus (RSV), Bovine respiratory syncytial virus, Pneumonia virus of mice, or Turkey rhinotracheitis virus. The Paramyxovirus may be Parainfluenza virus types 1-4 (PIV), Mumps, Sendai viruses, Simian virus 5, Bovine parainfluenza virus, Nipahvirus, Henipavirus or Newcastle disease virus. The Poxviridae may be Variola vera, for example Variola major and Variola minor. The Metapneumovirus may be human metapneumovirus (hMPV) or avian metapneumoviruses (aMPV). The Morbillivirus may be measles. The Picornaviruses may be Enteroviruses, Rhinoviruses, Heparnavirus, Parechovirus, Cardioviruses and Aphthoviruses. The Enteroviruses may be Poliovirus types 1, 2 or 3, Coxsackie A virus types 1 to 22 and 24, Coxsackie B virus types 1 to 6, Echovirus (ECHO) virus) types 1 to 9, 11 to 27 and 29 to 34 or Enterovirus 68 to 71. The Bunyavirus may be California encephalitis virus. The Phlebovirus may be Rift Valley Fever virus. The Nairovirus may be Crimean-Congo hemorrhagic fever virus. The Heparnaviruses may be Hepatitis A virus (HAV). The Togaviruses may be Rubivirus. The Flavivirus may be Tick-borne encephalitis (TBE) virus, Dengue (types 1, 2, 3 or 4) virus, Yellow Fever virus, Japanese encephalitis virus, Kyasanur Forest Virus, West Nile encephalitis virus, St. Louis encephalitis virus, Russian spring-summer encephalitis virus or Powassan encephalitis virus. The Pestivirus may be Bovine viral diarrhea (BVDV), Classical swine fever (CSFV) or Border disease (BDV). The Hepadnavirus may be Hepatitis B virus or Hepatitis C virus. The Rhabdovirus may be Lyssavirus (Rabies virus) or Vesiculovirus (VSV). The Caliciviridae may be Norwalk virus, or Norwalk-like Viruses, such as Hawaii Virus and Snow Mountain Virus. The Coronavirus may be SARS CoV-1, SARS-CoV-2, MERS, Human respiratory coronavirus, Avian infectious bronchitis (IBV), Mouse hepatitis virus (MHV), or Porcine transmissible gastroenteritis virus (TGEV). The Retrovirus may be Oncovirus, a Lentivirus or a Spumavirus. The Reovirus may be an Orthoreo virus, a Rotavirus, an Orbivirus, or a Coltivirus. The Parvovirus may be Parvovirus B 19. The Human Herpesvirus may be Herpes Simplex Viruses (HSV), Varicella-zoster virus (VZV), Epstein-Barr virus (EBV), Cytomegalovirus (CMV), Human Herpesvirus 6 (HHV6), Human Herpesvirus 7 (HHV7), or Human Herpesvirus 8 (HHV8). The Papovavirus may be Papilloma viruses, Polyomaviruses, Adenoviruess or Arenaviruses.

The protein or peptide derived from bacteria may be a bacterial antigen.

The bacterial antigen may derived from a bacterium selected from the group consisting of: Neisseria meningitides, Streptococcus pneumoniae, Streptococcus pyogenes, Moraxella catarrhalis, Bordetella pertussis, Burkholderia sp. (e.g., Burkholderia mallei, Burkholderia pseudomallei and Burkholderia cepacia), Staphylococcus aureus, Haemophilus influenzae, Clostridium tetani (Tetanus), Clostridium perfringens, Clostridium botulinums, Cornynebacterium diphtheriae (Diphtheria), Pseudomonas aeruginosa, Legionella pneumophila, Coxiella burnetii, Brucella sp. (e.g., B. abortus, B. canis, B. melitensis, B. neotomae, B. ovis, B. suis and B. pinnipediae, Francisella sp. (e.g., F. novicida, F. philomiragia and F. tularensis), Streptococcus agalactiae, Neiserria gonorrhoeae, Chlamydia trachomatis, Treponema pallidum (Syphilis), Haemophilus ducreyi, Enterococcus faecalis, Enterococcus faecium, Helicobacter pylori, Staphylococcus saprophyticus, Yersinia enter ocolitica, E. coli, Bacillus anthracis (anthrax), Yersinia pestis (plague), Mycobacterium tuberculosis, Rickettsia, Listeria, Chlamydia pneumoniae, Vibrio cholerae, Salmonella typhi (typhoid fever), Borrelia burgdorfer, Porphyromonas s and Klebsiella sp.

The protein or peptide derived from a fungus may be a fungal antigen.

The fungal antigen may be derived from a fungus selected from the group consisting of Dermatophytres, including: Epidermophyton koccusum, Microsporum audouini, Microsporum canis, Microsporum distortum, Microsporum equinum, Microsporum gypsum, Microsporum nanum, Trichophyton concentricum, Trichophyton equinum, Trichophyton gallinae, Trichophyton gypseum, Trichophyton megnini, Trichophyton mentagrophytes, Trichophyton quinckeanum, Trichophyton rubrum, Trichophyton schoenleini, Trichophyton tonsurans, Trichophyton verrucosum, T verrucosum var. album, var. discoides, var. ochraceum, Trichophyton violaceum, and/or Trichophyton faviforme; or from Aspergillus fumigatus, Aspergillus kavus, Aspergillus niger, Aspergillus nidulans, Aspergillus terreus, Aspergillus sydowii, Aspergillus kavatus, Aspergillus glaucus, Blastoschizomyces capitatus, Candida albicans, Candida enolase, Candida tropicalis, Candida glabrata, Candida krusei, Candida parapsilosis, Candida stellatoidea, Candida kusei, Candida parakwsei, Candida lusitaniae, Candida pseudotropicalis, Candida guilliermondi, Cladosporium carrionii, Coccidioides immitis, Blastomyces dermatidis, Cryptococcus neoformans, Geotrichum clavatum, Histoplasma capsulatum, Klebsiella pneumoniae, Microsporidia, Encephalitozoon spp., Septata intestinalis and Enterocytozoon bieneusi; Brachiola spp, Microsporidium spp., Nosema spp., Pleistophora spp., Trachipleistophora spp., Vittaforma spp Paracoccidioides brasiliensis, Pneumocystis carinii, Pythiumn insidiosum, Pityrosporum ovale, Sacharomyces cerevisiae, Saccharomyces boulardii, Saccharomyces pombe, Scedosporium apiosperum, Sporothrix schenckii, Trichosporon beigelii, Toxoplasma gondii, Penicillium marneffei, Malassezia spp., Fonsecaea spp., Wangiella spp., Sporothrix spp., Basidiobolus spp., Conidiobolus spp., Rhizopus spp, Mucor spp, Absidia spp, Mortierella spp, Cunninghamella spp, Saksenaea spp., Alternaria spp, Curvularia spp, Helminthosporium spp, Fusarium spp, Aspergillus spp, Penicillium spp, Monolinia spp, Rhizoctonia spp, Paecilomyces spp, Pithomyces spp, and Cladosporium spp.

The protein or peptide derived from a protozoan may be a protozoan antigen.

The protozoan antigen may be derived from a protozoan selected from the group consisting of: Entamoeba histolytica, Giardia lambli, Cryptosporidium parvum, Cyclospora cayatanensis and Toxoplasma.

The therapeutic biomolecule may be a protein or peptide derived from a plant. Preferably, the protein or peptide is a plant antigen. For example, the plant antigen may be derived from Ricinus communis.

In another embodiment, the therapeutic biomolecule may be an immunogen or an antigen. Preferably, the immunogen or an antigen is a tumour immunogen or antigen, or cancer immunogen or antigen. The tumour immunogens and antigens may be peptide-containing tumour antigens, such as a polypeptide tumour antigen or glycoprotein tumour antigens.

The tumour antigens may be (a) full length molecules associated with cancer cells, (b) homologs and modified forms of the same, including molecules with deleted, added and/or substituted portions, and (c) fragments of the same.

Suitable tumour immunogens include: class I-restricted antigens recognized by CD8+ lymphocytes or class II-restricted antigens recognized by CD4+ lymphocytes.

The tumour antigen may be an antigen that is associated with a cancer selected from the group consisting of: a testis cancer, melanoma, lung cancer, head and neck cancer, NSCLC, breast cancer, gastrointestinal cancer, bladder cancer, colorectal cancer, pancreatic cancer, lymphoma, leukaemia, renal cancer, hepatoma, ovarian cancer, gastric cancer and prostate cancer.

The tumour antigen may be selected from:
(a) cancer-testis antigens, such as NY-ESO-I, SSX2, SCP-1, as well as RAGE, BAGE, GAGE and MAGE family polypeptides, for example, GAGE-I, GAGE-2, MAGE-I, MAGE-2, MAGE-3, MAGE-4, MAGE-5, MAGE-6, and MAGE-12 (which can be used, for example, to address melanoma, lung, head and neck, NSCLC, breast, gastrointestinal, and bladder tumours);
(b) mutated antigens, for example, p53 (associated with various solid tumours, e.g., colorectal, lung, head and neck cancer), p21/Ras (associated with, e.g., melanoma, pancreatic cancer and colorectal cancer), CDK4 (associated with, e.g., melanoma), MUM-1 (associated with, e.g., melanoma), caspase-8 (associated with, e.g., head and neck cancer), CIA 0205 (associated with, e.g., bladder cancer), HLA-A2-R1701, beta catenin (associated with, e.g., melanoma), TCR (associated with, e.g., T-cell non-Hodgkins lymphoma), BCR-abl (associated with, e.g., chronic myelogenous leukemia), triosephosphate isomerase, KIA 0205, CDC-27, and LDLR-FUT;
(c) over-expressed antigens, for example, Galectin 4 (associated with, e.g., colorectal cancer), Galectin 9 (associated with, e.g., Hodgkin's disease), proteinase 3 (associated with, e.g., chronic myelogenous leukemia), WT 1 (associated with, e.g., various leukaemias), carbonic anhydrase (associated with, e.g., renal cancer), aldolase A (associated with, e.g., lung cancer), PRAME (associated with, e.g., melanoma), HER-2/neu (associated with, e.g., breast, colon, lung and ovarian cancer), alpha-fetoprotein (associated with, e.g., hepatoma), KSA (associated with, e.g., colorectal cancer), gastrin (associated with, e.g., pancreatic and gastric cancer), telomerase catalytic protein, MUC-I (associated with, e.g., breast and ovarian cancer), G-250 (associated with, e.g., renal cell carcinoma), p53 (associated with, e.g., breast, colon cancer), and carcinoembryonic antigen (associated with, e.g., breast cancer, lung cancer, and cancers of the gastrointestinal tract such as colorectal cancer);
(d) shared antigens, for example, melanoma-melanocyte differentiation antigens, such as MART-1/Melan A, gp100, MC1R, melanocyte-stimulating hormone receptor, tyrosinase, tyrosinase related protein-1/TRPl and tyrosinase related protein-2/TRP2 (associated with, e.g., melanoma);
(e) prostate-associated antigens, such as PAP, PSA, PSMA, PSH-Pl, PSM-Pl, PSM-P2, associated with e.g., prostate cancer; and/or
(f) immunoglobulin idiotypes (associated with myeloma and B cell lymphomas, for example).

The therapeutic biomolecule may be a eukaryotic protein or peptide. In one embodiment, the eukaryotic protein or peptide is a mammalian protein or peptide. The mammalian protein or peptide may be selected from the group consisting of: an enzyme; an enzyme inhibitor; a hormone; an immune system protein; a receptor; a binding protein; a transcription factor; translation factor; tumour growth suppressing protein; a structural protein; and a blood protein.

The immune system protein may be an antibody or antigen binding fragment thereof. Accordingly, the therapeutic biomolecule may be an antibody or antigen binding fragment thereof. The antigen binding fragment may comprise an individual heavy or light chain, or a fragment thereof, such as VL, VH and Fd; a monovalent fragment, such as Fv, Fab, and Fab′; a bivalent fragment, such as F(ab′)₂; a single chain Fv (scFv); one or more complementarity determining region (CDR); or a Fc fragment.

The enzyme may be selected from the group consisting of: chymosin; gastric lipase; tissue plasminogen activator; streptokinase; a cholesterol biosynthetic or degradative steriodogenic enzyme; kinases; phosphodiesterases; methylases; de-methylases; dehydrogenases; cellulases; proteases; lipases; phospholipases; aromatases; cytochromes; adenylate or guanylate cyclases and neuramidases.

The enzyme inhibitor may be tissue inhibitor of metalloproteinase (TIMP). The hormone may be growth hormone.

The immune system protein may be selected from the group consisting of: a cytokine; a chemokine; a lymphokine; erythropoietin; an integrin; addressin; selectin; homing receptors; T cell receptors and immunoglobulins.

The cytokine may be an interleukin, for example IL-2, IL-4 and/or IL-6, colony stimulating factor (CSF), granulocyte colony stimulating factor (G-CSF), granulocyte-macrophage colony stimulating factor (GM-CSF) or tumour necrosis factor (TNF).

The chemokine may be a macrophage inflammatory protein-2 and/or a plasminogen activator.

The lymphokine may be an interferon.

The immunoglobulin may be a natural, modified or chimeric immunoglobulin or a fragment thereof. Preferably, the immunoglobulin is a chimeric immunoglobulin having dual activity such as antibody enzyme or antibody-toxin chimera.

The hormone may be selected from the group consisting of: insulin, thyroid hormone, catecholamines, gonadotrophines, trophic hormones, prolactin, oxytocin, dopamine, bovine somatotropin, leptins; growth hormones (e.g., human grown hormone), growth factors (e.g., epidermal growth factor, nerve growth factor, insulin-like growth factor and the like).

The receptor may be a steroid hormone receptor or a peptide receptor. Preferably, the receptor is a growth factor receptor.

The binding protein may be a growth factor binding protein.

The tumour growth suppressing protein may be a protein that inhibits angiogenesis.

The structural protein may be selected from the group consisting of: collagen; fibroin; fibrinogen; elastin; tubulin; actin; and myosin.

The blood protein may be selected from the group consisting of thrombin; serum albumin; Factor VII; Factor VIII; insulin; Factor IX; Factor X; tissue plasminogen activator; protein C; von Willebrand factor; antithrombin III; glucocerebrosidase; erythropoietin granulocyte colony stimulating factor (GCSF) or modified Factor VIII; and anticoagulants.

In one preferred embodiment, the therapeutic biomolecule is a cytokine which is capable of regulating lymphoid homeostasis, preferably a cytokine which is involved in and preferably induces or enhances development, priming, expansion, differentiation and/or survival of T cells. Thus, preferably, the cytokine is an interleukin. Most preferably, IL-2, IL-7, IL-12, IL-15, or IL-21.

The therapeutic biomolecule may be protein that is capable of enhancing reprogramming of somatic cells to cells having stem cell characteristics. The protein that is capable of enhancing reprogramming of somatic cells to cells having stem cell characteristics may be selected from the group consisting of: OCT4, SOX2, NANOG, LIN28, p53, ART-4, BAGE, ss-catenin/m, Bcr-abL CAMEL, CAP-1, CASP-8, CDC27/m, CD 4/m, CEA, CLAUDIN-12, c-MYC, CT, Cyp-B, DAM, ELF2M, ETV6-AML1, G250, GAGE, GnT-V, Gapioo, HAGE, HER-2/neu, HPV-E7, HPV-E6, HAST-2, hTERT (or hTRT), LAGE, LDLR/FUT, MAGE-A, MAGE-B, MAGE-C, MART-1/Melan-A, MC1R, Myosin/m, MUC1, MUM-1, -2, -3, NA88-A, NF1, NY-ESO-1, NY-BR-1, p190 minor BCR-abL, Plac-1, Pml/RARa, PRAME, proteinase 3, PSA, PSM, RAGE, RU1 or RU2, SAGE, SART-1 or SART-3, SCGB3A2, SCP1, SCP2, SCP3, SSX, SURVIVIN, TEL/AML1, TPI/m, TRP-1, TRP-2, TRP-2/INT2, TPTE and WT, preferably WT-1.

Preferably, MAGE-A is selected from the group consisting of: MAGE-A 1, MAGE-A2, MAGE-A3, MAGE-A4, MAGE-A5, MAGE-A6, MAGE-A7, MAGE-A8, MAGE-A9, MAGE-A 10, MAGE-A 11, or MAGE-A 12.

Preferably, the protein that is capable of enhancing reprogramming of somatic cells to cells having stem cell characteristics is OCT4, SOX2, LF4; c-MYC; NANOG; LIN28.

The therapeutic biomolecule may be a biomolecule that is utilised for the modification of cells ex vivo for cell-therapy indications. Thus, preferably the therapeutic biomolecule may be selected from the group consisting of an immunoglobulin, a T-cell receptor and NK receptor.

The therapeutic biomolecule may be an RNA molecule that is capable of regulating expression of endogenous host genes, for example an interfering RNA, such as small RNA, siRNA or microRNA.

The sequence encoding the at least one non-viral innate modulatory protein (IMP) may be disposed anywhere within the RNA construct of the first aspect, such that the sequence encoding the therapeutic biomolecule (i.e. the GOI in FIG. 1) may be disposed either 5′ or 3′ to the sequence encoding the at least one innate modulatory protein.

For example, in one embodiment, the sequence encoding the therapeutic biomolecule is preferably disposed 5′ to the sequence encoding the at least one innate modulatory protein. See for example, the saRNA embodiments 2a, 3a, 4a, and the mRNA embodiments 6a and 7a shown in FIG. 1.

However, in another embodiment, the sequence encoding the therapeutic biomolecule is preferably disposed 3′ to the sequence encoding the at least one innate modulatory protein. See for example, the saRNA embodiments 2b, 3b, 4b, and the mRNA embodiments 6b and 7b shown in FIG. 1.

Preferably, the RNA construct according to the first aspect comprises at least one promotor, which may be either genomic or subgenomic. Preferably, however, the promoter is a subgenomic promoter, as is shown in FIG. 1 (embodiments 1-4b). Preferably, therefore, saRNA constructs of the invention comprise a promoter. The skilled person would understand that the subgenomic promotor relates to a promoter that is operably linked to the sequences encoding the at least one therapeutic biomolecule and the at least one innate inhibitor protein, such that it enables the transcription of the nucleotide sequence encoding the therapeutic biomolecule and the at least one innate modulatory protein.

Preferably, the subgenomic promoter is 26S, which is provided herein as SEQ ID No: 204, as follows:

[SEQ ID No: 204]
GGGCCCCTATAACTCTCTACGGCTAACCTGAATGGACTACGACAT

Accordingly, preferably the promoter (which is preferably a subgenomic promoter) is as substantially as set out in SEQ ID NO: 204, or a variant or fragment thereof.

In one embodiment, the same promotor is operably linked to the sequence encoding the at least therapeutic biomolecule and the sequence encoding the at least one innate modulatory protein.

The inventor's designs, wherein both the therapeutic biomolecule (i.e. GOI) and IMP are encoded by a single strand of RNA, advantageously enables the use of much smaller doses of RNA, because it ensures that the protein is being expressed in the same cell that is sensing the RNA, and can also be replicated, therefore having the additional aspect of expression and amplification of the innate modulatory component.

Thus, in one embodiment of the RNA construct, the promoter is disposed 5′ of the sequence encoding the at least one therapeutic biomolecule and the sequence encoding the at least one innate inhibitor protein, such that the promoter is operably linked to both sequences, thereby driving expression of both.

In another embodiment, however, a first promotor is operably linked to the sequence encoding the at least one therapeutic biomolecule, and a second promotor is operably linked the sequence encoding the at least one innate inhibitor protein.

The RNA construct may encode at least two, three, four or five IMPs. In embodiments in which there is more than one sequence encoding an innate modulatory protein, a single promotor may be operably linked to all sequences encoding an innate modulatory protein. Alternatively, a promotor may be linked to each of the sequences encoding an innate modulatory protein, such that each innate modulatory protein is operably linked to a separate promoter. In this embodiment, the separate promoters may comprise the same promotor sequence or different promoter sequences. In another embodiment, different promotors are operably linked to each sequence encoding an innate modulatory protein.

The RNA construct may further comprise a linker sequence disposed between the sequence encoding the at least one therapeutic biomolecule and the sequence encoding the at least one innate modulatory protein. This linker sequence is such that it allows the production of the IMP and the production of the therapeutic molecule from the single promoter. In one embodiment, the linker sequence encodes a peptide linker that is configured to be digested or cleaved following translation, to thereby separate the at least one therapeutic biomolecule and the at least one innate modulatory protein in the host cell. As such, the linker sequence is preferably a cleavable peptide, which may form a cleavage site, for example a 2A peptide (Furler S, Paterna J-C, Weibel M and Bueler H Recombinant AAV vectors containing the foot and mouth disease virus 2A sequence confer efficient bicistronic gene expression in cultured cells and rat substantia nigra neurons Gene Ther. 2001, vol. 8, PP: 864-873).

Preferably, the linker sequence encoding the 2A peptide sequence connects the two coding sequences together. This enables the RNA construct to overcome the size restrictions that may occur with expression in various vectors and enables expression and translation of all the peptides encoded by the RNA construct of the first aspect to occur under control of a single promoter, as a single protein. Thus, following the translation of the single protein comprising the sequences of the IMP, the 2A peptide, and the therapeutic biomolecule, cleavage occurs in the viral 2A peptide sequence at the terminal glycine-proline link, thereby liberating two polypeptides.

The 2A spacer sequence may be any known variant, which includes those sequences referred to as E2A, F2A, P2A and T2A, as disclosed in Wang Y et al. Scientific Reports 2015, 5, i.e. suitable 2A peptides include the porcine teschovirus-1 2A (P2A)—ATNFSLLKQAGDVEENPGP (SEQ ID No: 205), Thosea asigna virus 2A (T2A)−QCTNYALLKLAGDVESNPGP (SEQ ID No: 206), equine rhinitis A virus 2A (E2A), and Foot and mouth disease virus 2A (F2A) VKQTLNFDLLKLAGDVESNPGP (SEQ ID No: 207). Preferably, the 2A peptide is Thosea asigna virus 2A (T2A).

In another embodiment, the cleavable peptide is a self-cleaving peptide. In an embodiment, the linker comprises a viral 2A peptide spacer and further comprises a furin cleavage site. Preferably, the self-cleaving peptide is a furin/2A peptide. Insertion of an upstream furin cleavage site allows the removal of 2A residues that would otherwise remain attached to the upstream protein.

The furin sequence may be disposed 3′ or 5′ of the 2A sequence. Preferably, however, the furin sequence is disposed 5′ of the 2A sequence, and preferably with a GSG spacer disposed between the furin and 2A sequence.

The skilled person would appreciate that furin is a ubiquitous calcium-dependent proprotein convertase located in the secretory pathway (mainly in the golgi and trans-golgi network) that cleaves precursor proteins at a specific recognition sequence−canonically R-X-R/K/X-R (SEQ ID No: 208), and cleaving the proprotein after the final R. Thus, in one embodiment the furin sequence is R-X-R/K/X-R. However, preferably, the furin sequence is the optimised sequence RRRRRR (SEQ ID No: 209) a GSG sequence. A five R variant embodiment is also envisaged. Preferably, the GSG spacer is disposed 3′ of the furin sequence and 5′ of the 2A sequence.

Thus, preferably, the spacer sequence is the furin/T2A, as provided by NCBI Reference Sequence: GenBank: AAC97195.1, and provided herein as SEQ ID No: 210, as follows:

[SEQ ID No: 210]
RRRRRRGSGEGRGSLLTCGDVEENPGP

Hence, preferably the spacer sequence comprises an amino acid sequence substantially as set out in SEQ ID NO: 210, or a variant or fragment thereof. FIG. 1 shows embodiments 2a, 2b and 6a, 6b in which the GOI and IMP are linked by a nucleotide sequence which encodes the Furin-T2a cleavage site. In one embodiment, shown as either 2a or 6a in FIG. 1, the F-T2a cleavage site separates a 5′ GOI and a 3′ IMP. In one embodiment, shown as either 2b or 6b in FIG. 1, the F-T2a cleavage site separates a 3′ GOI and a 5′ IMP.

In embodiments in which the RNA construct or replicon comprises more than one sequence encoding an innate modulatory protein, the construct may comprise linker sequences disposed between each sequence encoding an innate modulatory protein, or only between some IMPs.

In one embodiment, the sequence encoding the at least one therapeutic biomolecule and the sequence encoding the at least one innate modulatory protein may be separated by a stop codon followed by an internal ribosome entry site (IRES) sequence capable of initiating translation of the downstream sequence, whichever sequence that may be (i.e. GOI or IMP as shown in embodiments 3a, 3b, 7a or 7b in FIG. 1). Therefore, preferably the IRES sequence is disposed between the sequence encoding the at least one therapeutic biomolecule and the sequence encoding at least one innate modulatory protein. Where multiple sequences encoding at least one innate modulatory protein are used, linker sequences may include combinations of known cleavage sequences and/or IRES sequences. In one embodiment, shown as either 3a or 7a in FIG. 1, the IRES site separates a 5′ GOI and a 3′ IMP. In one embodiment, shown as either 3b or 7b in FIG. 1, the IRES site separates a 3′ GOI and a 5′ IMP.

In an embodiment, the IRES is a picornavirus IRES. Oher typical IRES sequences include those such as the IRES sequence of encephalomyocarditis virus (EMCV) or vascular endothelial growth factor and type 1 collagen-inducible protein (VCIP), and would be known to those skilled in the Art.

In other embodiments, the IRES may be selected from a rhinovirus IRES, a hepatitis A virus IRES, a hepatitis C virus IRES, a poliovirus IRES, an enterovirus IRES, a cardiovirus IRES, an aphthovirus IRES, flavivirus IRES, a pestivirus IRES, a cripavirus IRES, a Rhopalosiphum padi virus IRES, or any suitable IRES. In particular, the IRES may be any IRES described by the “IRESite” which provides a database of experimentally verified IRES structures (http://www.iresite.org/), or as disclosed in “New Messenger RNA Research Communications” (ISBN: 1-60021-488-6).

In a preferred embodiment, the IRES is a foot-and-mouth disease virus (FMDV) IRES, which may be as set out in SEQ ID No:211, or a fragment or variant thereof, as follows:

[SEQ ID NO: 211]
AGCAGGTTTCCCCAACTGACACAAAACGTGCAACTTGAAA
CTCCGCCTGGTCTTTCCAGGTCTAGAGGGGTAACACTTTG
TACTGCGTTTGGCTCCACGCTCGATCCACTGGCGAGTGTT
AGTAACAGCACTGTTGCTTCGTAGCGGAGCATGACGGCCG
TGGGAACTCCTCCTTGGTAACAAGGACCCACGGGGCCAAA
AGCCACGCCCACACGGGCCCGTCATGTGTGCAACCCCAGC
ACGGCGACTTTACTGCGAAACCCACTTTAAAGTGACATTG
AAACTGGTACCCACACACTGGTGACAGGCTAAGGATGCCC
TTCAGGTACCCCGAGGTAACACGCGACACTCGGGATCTGA
GAAGGGGACTGGGGCTTCTATAAAAGCGCTCGGTTTAAAA
AGCTTCTATGCCTGAATAGGTGACCGGAGGTCGGCACCTT
TCCTTTGCAATTACTGACCAC

In another preferred embodiment, the IRES is an encephalomyocarditis virus (EMCV) IRES. The EMCV IRES may be as set out in SEQ ID No:212, or a fragment or variant thereof, as follows:

[SEQ ID NO: 212]
CGTTACTGGCCGAAGCCGCTTGGAATAAGGCCGGTGTGCG
TTTGTCTATATGTTATTTTCCACCATATTGCCGTCTTTTG
GCAATGTGAGGGCCCGGAAACCTGGCCCTGTCTTCTTGAC
GAGCATTCCTAGGGGTCTTTCCCCTCTCGCCAAAGGAATG
CAAGGTCTGTTGAATGTCGTGAAGGAAGCAGTTCCTCTGG
AAGCTTCTTGAAGACAAACAACGTCTGTAGCGACCCTTTG
CAGGCAGCGGAACCCCCCACCTGGCGACAGGTGCCTCTGC
GGCCAAAAGCCACGTGTATAAGATACACCTGCAAAGGCGG
CACAACCCCAGTGCCACGTTGTGAGTTGGATAGTTGTGGA
AAGAGTCAAATGGCTCCCCTCAAGCGTATTCAACAAGGGG
CTGAAGGATGCCCAGAAGGTACCCCATTGTATGGGATCTG
ATCTGGGGCCTCGGTGCACATGCTTTTCATGTGTTTAGTC
GAGGTTAAAAAACGTCTAGGCCCCCCGAACCACGGGGACG
TGGTTTTCCTTTGAAAAACACGATGATAATA

Therefore, preferably the IRES comprises a nucleotide sequence substantially as set out in SEQ ID No: 211 or 212, or a fragment or variant thereof.

Alternatively, instead of an IRES or a 2A linker, the linker sequence may comprise a sequence encoding a flexible linker, which allows for the expression of both the therapeutic biomolecule and IMP as a single polypeptide chain, but wherein the therapeutic biomolecule and IMP act as independent proteins. Hence, the proteins exert their effects in the same manner as if they were singly expressed. The flexible linker sequence may be as disclosed by WO 2013/061076 A1 (Oxford Biomedica). The flexible linker sequence may be referred to herein as SEQ ID No:213, or a fragment or variant thereof, as follows:

[SEQ ID NO: 213]
GGAGGTGGCGGGTCCGGGGGCGGGGGTAGCGGTGGCGGGGGCTCC

Preferably, therefore, the flexible linker sequence comprises a nucleotide sequence substantially as set out in SEQ ID No: 213, or a fragment or variant thereof.

In one preferred embodiment, the flexible linker sequence comprises a nucleotide sequence encoding an amino acid sequence referred to herein as SEQ ID NO: 214, or a fragment or variant thereof, as set out below:

[SEQ ID NO: 214]
GGGGSGGGGSGGGGS

Preferably, therefore, the flexible linker sequence encodes an amino acid sequence substantially as set out in SEQ ID No: 214, or a fragment or variant thereof.

In yet another embodiment, the sequence encoding the at least one therapeutic biomolecule and the at least one innate inhibitor protein may be separated by a stop codon followed by a second subgenomic promotor sequence capable of initiating transcription of the downstream sequence. Examples of this embodiment are illustrated in FIG. 1, embodiments 4a and 4b.

The RNA construct (preferably when it is a saRNA construct) may encode at least one non-structural protein (NSP), disposed 5′ or 3′ of the sequence encoding the at least one therapeutic biomolecule and the at least one innate modulatory protein. Preferably, the sequence encoding the at least one NSP is disposed 5′ of the sequences encoding the therapeutic biomolecule and the at least one innate modulatory protein. Thus, preferably the sequence encoding the at least one NSP is disposed at the 5′ end of the RNA construct.

The at least one non-structural protein, which is encoded by the RNA construct, may be the RNA polymerase NSP4. The one or more non-structural protein preferably encodes a replicase. Preferably, the construct encodes NSP1, NSP2, NSP3 and NSP4. The skilled person would understand that nsP1 is the viral capping enzyme and membrane anchor of the replication complex (RC), while NSP2 is an RNA helicase and the protease responsible for the ns polyprotein processing. NSP3 interacts with several host proteins and may modulate protein poly- and mono-ADP-ribosylation, and NSP4 is the core viral RNA-dependent RNA polymerase.

In one embodiment, NSP1 is provided herein as SEQ ID No: 215, as follows:

[SEQ ID No: 215]
MEKVHVDIEEDSPFLRALQRSFPQFEVEAKQVTDNDHANA
RAFSHLASKLIETEVDPSDTILDIGSAPARRMYSKHKYHC
ICPMRCAEDPDRLYKYATKLKKNCKEITDKELDKKMKELA
AVMSDPDLETETMCLHDDESCRYEGQVAVYQDVYAVDGPT
SLYHQANKGVRVAYWIGFDTTPFMFKNLAGAYPSYSTNWA
DETVLTARNIGLCSSDVMERSRRGMSILRKKYLKPSNNVL
FSVGSTIYHEKRDLLRSWHLPSVFHLRGKQNYTCRCETIV
SCDGYVVKRIAISPGLYGKPSGYAATMHREGFLCCKVTDT
LNGERVSFPVCTYVPATLCDQMTGILATDVSADDAQKLLV
GLNQRIVVNGRTQRNTNTMKNYLLPVVAQAFARWAKEYKE
DQEDERPLGLRDRQLVMGCCWAFRRHKITSIYKRPDTQTI
IKVNSDFHSFVLPRIGSNTLEIGLRTRIRKMLEEHKEPSP
LITAEDVQEAKCAADEAKEVREAEELRAALPPLAADVEEP
TLEADVDLMLQEAGA

Accordingly, NSP1 preferably comprises an amino acid sequence as substantially as set out in SEQ ID No: 215, or a biologically active variant or fragment thereof.

In one embodiment, NSP1 is encoded by a nucleotide sequence a defined in SEQ ID No: 216, as follows:

[SEQ ID No: 216]
ATGGAGAAAGTTCACGTTGACATCGAGGAAGACAGCCCAT
TCCTCAGAGCTTTGCAGCGGAGCTTCCCGCAGTTTGAGGT
AGAAGCCAAGCAGGTCACTGATAATGACCATGCTAATGCC
AGAGCGTTTTCGCATCTGGCTTCAAAACTGATCGAAACGG
AGGTGGACCCATCCGACACGATCCTTGACATTGGAAGTGC
GCCCGCCCGCAGAATGTATTCTAAGCACAAGTATCATTGT
ATCTGTCCGATGAGATGTGCGGAAGATCCGGACAGATTGT
ATAAGTATGCAACTAAGCTGAAGAAAAACTGTAAGGAAAT
AACTGATAAGGAATTGGACAAGAAAATGAAGGAGCTGGCC
GCCGTCATGAGCGACCCTGACCTGGAAACTGAGACTATGT
GCCTCCACGACGACGAGTCGTGTCGCTACGAAGGGCAAGT
CGCTGTTTACCAGGATGTATACGCGGTTGACGGACCGACA
AGTCTCTATCACCAAGCCAATAAGGGAGTTAGAGTCGCCT
ACTGGATAGGCTTTGACACCACCCCTTTTATGTTTAAGAA
CTTGGCTGGAGCATATCCATCATACTCTACCAACTGGGCC
GACGAAACCGTGTTAACGGCTCGTAACATAGGCCTATGCA
GCTCTGACGTTATGGAGCGGTCACGTAGAGGGATGTCCAT
TCTTAGAAAGAAGTATTTGAAACCATCCAACAATGTTCTA
TTCTCTGTTGGCTCGACCATCTACCACGAGAAGAGGGACT
TACTGAGGAGCTGGCACCTGCCGTCTGTATTTCACTTACG
TGGCAAGCAAAATTACACATGTCGGTGTGAGACTATAGTT
AGTTGCGACGGGTACGTCGTTAAAAGAATAGCTATCAGTC
CAGGCCTGTATGGGAAGCCTTCAGGCTATGCTGCTACGAT
GCACCGCGAGGGATTCTTGTGCTGCAAAGTGACAGACACA
TTGAACGGGGAGAGGGTCTCTTTTCCCGTGTGCACGTATG
TGCCAGCTACATTGTGTGACCAAATGACTGGCATACTGGC
AACAGATGTCAGTGCGGACGACGCGCAAAAACTGCTGGTT
GGGCTCAACCAGCGTATAGTCGTCAACGGTCGCACCCAGA
GAAACACCAATACCATGAAAAATTACCTTTTGCCCGTAGT
GGCCCAGGCATTTGCTAGGTGGGCAAAGGAATATAAGGAA
GATCAAGAAGATGAAAGGCCACTAGGACTACGAGATAGAC
AGTTAGTCATGGGGTGTTGTTGGGCTTTTAGAAGGCACAA
GATAACATCTATTTATAAGCGCCCGGATACCCAAACCATC
ATCAAAGTGAACAGCGATTTCCACTCATTCGTGCTGCCCA
GGATAGGCAGTAACACATTGGAGATCGGGCTGAGAACAAG
AATCAGGAAAATGTTAGAGGAGCACAAGGAGCCGTCACCT
CTCATTACCGCCGAGGACGTACAAGAAGCTAAGTGCGCAG
CCGATGAGGCTAAGGAGGTGCGTGAAGCCGAGGAGTTGCG
CGCAGCTCTACCACCTTTGGCAGCTGATGTTGAGGAGCCC
ACTCTGGAAGCCGATGTCGACTTGATGTTACAAGAGGCTG
GGGCC

Accordingly, NSP1 is preferably encoded by a nucleotide sequence as substantially as set out in SEQ ID No: 216, or a variant or fragment thereof.

Accordingly, therefore, preferably the RNA construct comprises an RNA nucleotide sequence substantially as set out as SEQ ID No: 217, or a variant or fragment thereof.

[SEQ ID No: 217]
AUGGAGAAAGUUCACGUUGACAUCGAGGAAGACAGCCCAUUCCUC
AGAGCUUUGCAGCGGAGCUUCCCGCAGUUUGAGGUAGAAGCCAAG
CAGGUCACUGAUAAUGACCAUGCUAAUGCCAGAGCGUUUUCGCAU
CUGGCUUCAAAACUGAUCGAAACGGAGGUGGACCCAUCCGACACG
AUCCUUGACAUUGGAAGUGCGCCCGCCCGCAGAAUGUAUUCUAAG
CACAAGUAUCAUUGUAUCUGUCCGAUGAGAUGUGCGGAAGAUCCG
GACAGAUUGUAUAAGUAUGCAACUAAGCUGAAGAAAAACUGUAAG
GAAAUAACUGAUAAGGAAUUGGACAAGAAAAUGAAGGAGCUGGCC
GCCGUCAUGAGCGACCCUGACCUGGAAACUGAGACUAUGUGCCUC
CACGACGACGAGUCGUGUCGCUACGAAGGGCAAGUCGCUGUUUAC
CAGGAUGUAUACGCGGUUGACGGACCGACAAGUCUCUAUCACCAA
GCCAAUAAGGGAGUUAGAGUCGCCUACUGGAUAGGCUUUGACACC
ACCCCUUUUAUGUUUAAGAACUUGGCUGGAGCAUAUCCAUCAUAC
UCUACCAACUGGGCCGACGAAACCGUGUUAACGGCUCGUAACAUA
GGCCUAUGCAGCUCUGACGUUAUGGAGCGGUCACGUAGAGGGAUG
UCCAUUCUUAGAAAGAAGUAUUUGAAACCAUCCAACAAUGUUCUA
UUCUCUGUUGGCUCGACCAUCUACCACGAGAAGAGGGACUUACUG
AGGAGCUGGCACCUGCCGUCUGUAUUUCACUUACGUGGCAAGCAA
AAUUACACAUGUCGGUGUGAGACUAUAGUUAGUUGCGACGGGUAC
GUCGUUAAAAGAAUAGCUAUCAGUCCAGGCCUGUAUGGGAAGCCU
UCAGGCUAUGCUGCUACGAUGCACCGCGAGGGAUUCUUGUGCUGC
AAAGUGACAGACACAUUGAACGGGGAGAGGGUCUCUUUUCCCGUG
UGCACGUAUGUGCCAGCUACAUUGUGUGACCAAAUGACUGGCAUA
CUGGCAACAGAUGUCAGUGCGGACGACGCGCAAAAACUGCUGGUU
GGGCUCAACCAGCGUAUAGUCGUCAACGGUCGCACCCAGAGAAAC
ACCAAUACCAUGAAAAAUUACCUUUUGCCCGUAGUGGCCCAGGCA
UUUGCUAGGUGGGCAAAGGAAUAUAAGGAAGAUCAAGAAGAUGAA
AGGCCACUAGGACUACGAGAUAGACAGUUAGUCAUGGGGUGUUGU
UGGGCUUUUAGAAGGCACAAGAUAACAUCUAUUUAUAAGCGCCCG
GAUACCCAAACCAUCAUCAAAGUGAACAGCGAUUUCCACUCAUUC
GUGCUGCCCAGGAUAGGCAGUAACACAUUGGAGAUCGGGCUGAGA
ACAAGAAUCAGGAAAAUGUUAGAGGAGCACAAGGAGCCGUCACCU
CUCAUUACCGCCGAGGACGUACAAGAAGCUAAGUGCGCAGCCGAU
GAGGCUAAGGAGGUGCGUGAAGCCGAGGAGUUGCGCGCAGCUCUA
CCACCUUUGGCAGCUGAUGUUGAGGAGCCCACUCUGGAAGCCGAU
GUCGACUUGAUGUUACAAGAGGCUGGGGCC

In one embodiment, NSP2 is provided herein as SEQ ID No: 218, as follows:

[SEQ ID No: 218]
GSVETPRGLIKVTSYDGEDKIGSYAVLSPQAVLKSEKLSCIHPLA
EQVIVITHSGRKGRYAVEPYHGKVVVPEGHAIPVQDFQALSESA
TIVYNEREFVNRYLHHIATHGGALNTDEEYYKTVKPSEHDGEYLY
DIDRKQCVKKELVTGLGLTGELVDPPFHEFAYESLRTRPAAPYQV
PTIGVYGVPGSGKSGIIKSAVIKKDLVVSAKKENCAEIIRDVKKM
KGLDVNARTVDSVLLNGCKHPVETLYIDEAFACHAGTLRALIAII
RPKKAVLCGDPKQCGFFNMMCLKVHFNHEICTQVFHKSISRRCTK
SVTSVVSTLFYDKKMRTTNPKETKIVIDTTGSTKPKQDDLILTCF
RGWVKQLQIDYKGNEIMTAAASQGLTRKGVYAVRYKVNENPLYAP
TSEHVNVLLTRTEDRIVWKTLAGDPWIKTLTAKYPGNFTATIEEW
QAEHDAIMRHILERPDPTDVFQNKANVCWAKALVPVLKTAGIDM
TTEQWNTVDYFETDKAHSAEIVLNQLCVRFFGLDLDSGLFSAPTV
PLSIRNNHWDNSPSPNMYGLNKEVVRQLSRRYPQLPRAVATGRVY
DMNTGTLRNYDPRINLVPVNRRLPHALVLHHNEHPQSDFSSFVSK
LKGRTVLVVGEKLSVPGKMVDWLSDRPEATFRARLDLGIPGDVPK
YDIIFVNVRTPYKYHHYQQCEDHAIKLSMLTKKACLHLNPGGTCV
SIGYGYADRASESIIGAIARQFKFSRVCKPKSSLEETEVLFVFIG
YDRKARTHNSYKLSSTLTNIYTGSRLHEAGC

Accordingly, nsP2 preferably comprises an amino acid sequence as substantially as set out in SEQ ID No: 218, or a biologically active variant or fragment thereof.

In one embodiment, NSP2 is encoded by a nucleotide sequence a defined in SEQ ID No: 219, as follows:

[SEQ ID No: 219]
GGCTCAGTGGAGACACCTCGTGGCTTGATAAAGGTTACCAGCTAC
GATGGCGAGGACAAGATCGGCTCTTACGCTGTGCTTTCTCCGCAG
GCTGTACTCAAGAGTGAAAAATTATCTTGCATCCACCCTCTCGCT
GAACAAGTCATAGTGATAACACACTCTGGCCGAAAAGGGCGTTAT
GCCGTGGAACCATACCATGGTAAAGTAGTGGTGCCAGAGGGACAT
GCAATACCCGTCCAGGACTTTCAAGCTCTGAGTGAAAGTGCCACC
ATTGTGTACAACGAACGTGAGTTCGTAAACAGGTACCTGCACCAT
ATTGCCACACATGGAGGAGCGCTGAACACTGATGAAGAATATTAC
AAAACTGTCAAGCCCAGCGAGCACGACGGCGAATACCTGTACGAC
ATCGACAGGAAACAGTGCGTCAAGAAAGAACTAGTCACTGGGCTA
GGGCTCACAGGCGAGCTGGTGGATCCTCCCTTCCATGAATTCGCC
TACGAGAGTCTGAGAACACGACCAGCCGCTCCTTACCAAGTACCA
ACCATAGGGGTGTATGGCGTGCCAGGATCAGGCAAGTCTGGCATC
ATTAAAAGCGCAGTCACCAAAAAAGATCTAGTGGTGAGCGCCAAG
AAAGAAAACTGTGCAGAAATTATAAGGGACGTCAAGAAAATGAAA
GGGCTGGACGTCAATGCCAGAACTGTGGACTCAGTGCTCTTGAAT
GGATGCAAACACCCCGTAGAGACCCTGTATATTGACGAAGCTTTT
GCTTGTCATGCAGGTACTCTCAGAGCGCTCATAGCCATTATAAGA
CCTAAAAAGGCAGTGCTCTGCGGGGATCCCAAACAGTGCGGTTTT
TTTAACATGATGTGCCTGAAAGTGCATTTTAACCACGAGATTTGC
ACACAAGTCTTCCACAAAAGCATCTCTCGCCGTTGCACTAAATCT
GTGACTTCGGTCGTCTCAACCTTGTTTTACGACAAAAAAATGAGA
ACGACGAATCCGAAAGAGACTAAGATTGTGATTGACACTACCGGC
AGTACCAAACCTAAGCAGGACGATCTCATTCTCACTTGTTTCAGA
GGGTGGGTGAAGCAGTTGCAAATAGATTACAAAGGCAACGAAATA
ATGACGGCAGCTGCCTCTCAAGGGCTGACCCGTAAAGGTGTGTAT
GCCGTTCGGTACAAGGTGAATGAAAATCCTCTGTACGCACCCACC
TCAGAACATGTGAACGTCCTACTGACCCGCACGGAGGACCGCATC
GTGTGGAAAACACTAGCCGGCGACCCATGGATAAAAACACTGACT
GCCAAGTACCCTGGGAATTTCACTGCCACGATAGAGGAGTGGCAA
GCAGAGCATGATGCCATCATGAGGCACATCTTGGAGAGACCGGAC
CCTACCGACGTCTTCCAGAATAAGGCAAACGTGTGTTGGGCCAAG
GCTTTAGTGCCGGTGCTGAAGACCGCTGGCATAGACATGACCACT
GAACAATGGAACACTGTGGATTATTTTGAAACGGACAAAGCTCAC
TCAGCAGAGATAGTATTGAACCAACTATGCGTGAGGTTCTTTGGA
CTCGATCTGGACTCCGGTCTATTTTCTGCACCCACTGTTCCGTTA
TCCATTAGGAATAATCACTGGGATAACTCCCCGTCGCCTAACATG
TACGGGCTGAATAAAGAAGTGGTCCGTCAGCTCTCTCGCAGGTAC
CCACAACTGCCTCGGGCAGTTGCCACTGGAAGAGTCTATGACATG
AACACTGGTACACTGCGCAATTATGATCCGCGCATAAACCTAGTA
CCTGTAAACAGAAGACTGCCTCATGCTTTAGTCCTCCACCATAAT
GAACACCCACAGAGTGACTTTTCTTCATTCGTCAGCAAATTGAAG
GGCAGAACTGTCCTGGTGGTCGGGGAAAAGTTGTCCGTCCCAGGC
AAAATGGTTGACTGGTTGTCAGACCGGCCTGAGGCTACCTTCAGA
GCTCGGCTGGATTTAGGCATCCCAGGTGATGTGCCCAAATATGAC
ATAATATTTGTTAATGTGAGGACCCCATATAAATACCATCACTAT
CAGCAGTGTGAAGACCATGCCATTAAGCTTAGCATGTTGACCAAG
AAAGCTTGTCTGCATCTGAATCCCGGCGGAACCTGTGTCAGCATA
GGTTATGGTTACGCTGACAGGGCCAGCGAAAGCATCATTGGTGCT
ATAGCGCGGCAGTTCAAGTTTTCCCGGGTATGCAAACCGAAATCC
TCACTTGAAGAGACGGAAGTTCTGTTTGTATTCATTGGGTACGAT
CGCAAGGCCCGTACGCACAATTCTTACAAGCTTTCATCAACCTTG
ACCAACATTTATACAGGTTCCAGACTCCACGAAGCCGGATGT

Accordingly, preferably NSP2 is encoded by a nucleotide sequence as substantially as 55 set out in SEQ ID No: 219, or a variant or fragment thereof.

Thus, the RNA construct may comprise SEQ ID No: 220, as follows:

[SEQ ID No: 220]
GGCUCAGUGGAGACACCUCGUGGCUUGAUAAAGGUUACCAGCUAC
GAUGGCGAGGACAAGAUCGGCUCUUACGCUGUGCUUUCUCCGCAG
GCUGUACUCAAGAGUGAAAAAUUAUCUUGCAUCCACCCUCUCGCU
GAACAAGUCAUAGUGAUAACACACUCUGGCCGAAAAGGGCGUUAU
GCCGUGGAACCAUACCAUGGUAAAGUAGUGGUGCCAGAGGGACAU
GCAAUACCCGUCCAGGACUUUCAAGCUCUGAGUGAAAGUGCCACC
AUUGUGUACAACGAACGUGAGUUCGUAAACAGGUACCUGCACCAU
AUUGCCACACAUGGAGGAGCGCUGAACACUGAUGAAGAAUAUUAC
AAAACUGUCAAGCCCAGCGAGCACGACGGCGAAUACCUGUACGAC
AUCGACAGGAAACAGUGCGUCAAGAAAGAACUAGUCACUGGGCUA
GGGCUCACAGGCGAGCUGGUGGAUCCUCCCUUCCAUGAAUUCGCC
UACGAGAGUCUGAGAACACGACCAGCCGCUCCUUACCAAGUACCA
ACCAUAGGGGUGUAUGGCGUGCCAGGAUCAGGCAAGUCUGGCAUC
AUUAAAAGCGCAGUCACCAAAAAAGAUCUAGUGGUGAGCGCCAAG
AAAGAAAACUGUGCAGAAAUUAUAAGGGACGUCAAGAAAAUGAAA
GGGCUGGACGUCAAUGCCAGAACUGUGGACUCAGUGCUCUUGAAU
GGAUGCAAACACCCCGUAGAGACCCUGUAUAUUGACGAAGCUUUU
GCUUGUCAUGCAGGUACUCUCAGAGCGCUCAUAGCCAUUAUAAGA
CCUAAAAAGGCAGUGCUCUGCGGGGAUCCCAAACAGUGCGGUUUU
UUUAACAUGAUGUGCCUGAAAGUGCAUUUUAACCACGAGAUUUGC
ACACAAGUCUUCCACAAAAGCAUCUCUCGCCGUUGCACUAAAUCU
GUGACUUCGGUCGUCUCAACCUUGUUUUACGACAAAAAAAUGAGA
ACGACGAAUCCGAAAGAGACUAAGAUUGUGAUUGACACUACCGGC
AGUACCAAACCUAAGCAGGACGAUCUCAUUCUCACUUGUUUCAGA
GGGUGGGUGAAGCAGUUGCAAAUAGAUUACAAAGGCAACGAAAUA
AUGACGGCAGCUGCCUCUCAAGGGCUGACCCGUAAAGGUGUGUAU
GCCGUUCGGUACAAGGUGAAUGAAAAUCCUCUGUACGCACCCACC
UCAGAACAUGUGAACGUCCUACUGACCCGCACGGAGGACCGCAUC
GUGUGGAAAACACUAGCCGGCGACCCAUGGAUAAAAACACUGACU
GCCAAGUACCCUGGGAAUUUCACUGCCACGAUAGAGGAGUGGCAA
GCAGAGCAUGAUGCCAUCAUGAGGCACAUCUUGGAGAGACCGGAC
CCUACCGACGUCUUCCAGAAUAAGGCAAACGUGUGUUGGGCCAAG
GCUUUAGUGCCGGUGCUGAAGACCGCUGGCAUAGACAUGACCACU
GAACAAUGGAACACUGUGGAUUAUUUUGAAACGGACAAAGCUCAC
UCAGCAGAGAUAGUAUUGAACCAACUAUGCGUGAGGUUCUUUGGA
CUCGAUCUGGACUCCGGUCUAUUUUCUGCACCCACUGUUCCGUUA
UCCAUUAGGAAUAAUCACUGGGAUAACUCCCCGUCGCCUAACAUG
UACGGGCUGAAUAAAGAAGUGGUCCGUCAGCUCUCUCGCAGGUAC
CCACAACUGCCUCGGGCAGUUGCCACUGGAAGAGUCUAUGACAUG
AACACUGGUACACUGCGCAAUUAUGAUCCGCGCAUAAACCUAGUA
CCUGUAAACAGAAGACUGCCUCAUGCUUUAGUCCUCCACCAUAAU
GAACACCCACAGAGUGACUUUUCUUCAUUCGUCAGCAAAUUGAAG
GGCAGAACUGUCCUGGUGGUCGGGGAAAAGUUGUCCGUCCCAGGC
AAAAUGGUUGACUGGUUGUCAGACCGGCCUGAGGCUACCUUCAGA
GCUCGGCUGGAUUUAGGCAUCCCAGGUGAUGUGCCCAAAUAUGAC
AUAAUAUUUGUUAAUGUGAGGACCCCAUAUAAAUACCAUCACUAU
CAGCAGUGUGAAGACCAUGCCAUUAAGCUUAGCAUGUUGACCAAG
AAAGCUUGUCUGCAUCUGAAUCCCGGCGGAACCUGUGUCAGCAUA
GGUUAUGGUUACGCUGACAGGGCCAGCGAAAGCAUCAUUGGUGCU
AUAGCGCGGCAGUUCAAGUUUUCCCGGGUAUGCAAACCGAAAUCC
UCACUUGAAGAGACGGAAGUUCUGUUUGUAUUCAUUGGGUACGAU
CGCAAGGCCCGUACGCACAAUUCUUACAAGCUUUCAUCAACCUUG
ACCAACAUUUAUACAGGUUCCAGACUCCACGAAGCCGGAUGU

Accordingly, therefore, preferably the RNA construct comprises an RNA nucleotide sequence substantially as set out as SEQ ID No: 220, or a variant or fragment thereof.

In one embodiment, NSP3 is provided herein as SEQ ID No: 221, as follows:

[SEQ ID No: 221]
APSYHVVRGDIATATEGVIINAANSKGQPGGGVCGALYKKEPES
FDLQPIEVGKARLVKGAAKHIIHAVGPNFNKVSEVEGDKQLAEA
YESIAKIVNDNNYKSVAIPLLSTGIFSGNKDRLTQSLNHLLTALD
TTDADVAIYCRDKKWEMTLKEAVARREAVEEICISDDSSVTEPDA
ELVRVHPKSSLAGRKGYSTSDGKTFSYLEGTKFHQAAKDIAEINA
MWPVATEANEQVCMYILGESMSSIRSKCPVEESEASTPPSTLPCL
CIHAMTPERVQRLKASRPEQITVCSSFPLPKYRITGVQKIQCSQP
ILFSPKVPAYIHPRKYLVETPPVDETPEPSAENQSTEGTPEQPPL
ITEDETRTRTPEPIIIEEEEEDSISLLSDGPTHQVLQVEADIHGP
PSVSSSSWSIPHASDFDVDSLSILDTLEGASVTSGATSAETNSYF
AKSMEFLARPVPAPRTVERNPPHPAPRTRIPSLAPSRACSRTSLV
STPPGVNRVITREELEALTPSRTPSRSVSRTSLVSNPPGVNRVIT
REEFEAFVAQQQRFDAGA

Accordingly, preferably nsP3 comprises an amino acid sequence as substantially as set out in SEQ ID No: 221, or a biologically active variant or fragment thereof.

In one embodiment, NSP3 is encoded by a nucleotide sequence a defined in SEQ ID No: 222, as follows:

[SEQ ID No: 222]
GCACCCTCATATCATGTGGTGCGAGGGGATATTGCCACGGCCACC
GAAGGAGTGATTATAAATGCTGCTAACAGCAAAGGACAACCTGGC
GGAGGGGTGTGCGGAGCGCTGTATAAGAAATTCCCGGAAAGCTTC
GATTTACAGCCGATCGAAGTAGGAAAAGCGCGACTGGTCAAAGGT
GCAGCTAAACATATCATTCATGCCGTAGGACCAAACTTCAACAAA
GTTTCGGAGGTTGAAGGTGACAAACAGTTGGCAGAGGCTTATGAG
TCCATCGCTAAGATTGTCAACGATAACAATTACAAGTCAGTAGCG
ATTCCACTGTTGTCCACCGGCATCTTTTCCGGGAACAAAGATCGA
CTAACCCAATCATTGAACCATTTGCTGACAGCTTTAGACACCACT
GATGCAGATGTAGCCATATACTGCAGGGACAAGAAATGGGAAATG
ACTCTCAAGGAAGCAGTGGCTAGGAGAGAAGCAGTGGAGGAGATA
TGCATATCCGACGACTCTTCAGTGACAGAACCTGATGCAGAGCTG
GTGAGGGTGCATCCGAAGAGTTCTTTGGCTGGAAGGAAGGGCTAC
AGCACAAGCGATGGCAAAACTTTCTCATATTTGGAAGGGACCAAG
TTTCACCAGGCGGCCAAGGATATAGCAGAAATTAATGCCATGTGG
CCCGTTGCAACGGAGGCCAATGAGCAGGTATGCATGTATATCCTC
GGAGAAAGCATGAGCAGTATTAGGTCGAAATGCCCCGTCGAAGAG
TCGGAAGCCTCCACACCACCTAGCACGCTGCCTTGCTTGTGCATC
CATGCCATGACTCCAGAAAGAGTACAGCGCCTAAAAGCCTCACGT
CCAGAACAAATTACTGTGTGCTCATCCTTTCCATTGCCGAAGTAT
AGAATCACTGGTGTGCAGAAGATCCAATGCTCCCAGCCTATATTG
TTCTCACCGAAAGTGCCTGCGTATATTCATCCAAGGAAGTATCTC
GTGGAAACACCACCGGTAGACGAGACTCCGGAGCCATCGGCAGAG
AACCAATCCACAGAGGGGACACCTGAACAACCACCACTTATAACC
GAGGATGAGACCAGGACTAGAACGCCTGAGCCGATCATCATCGAA
GAGGAAGAAGAGGATAGCATAAGTTTGCTGTCAGATGGCCCGACC
CACCAGGTGCTGCAAGTCGAGGCAGACATTCACGGGCCGCCCTCT
GTATCTAGCTCATCCTGGTCCATTCCTCATGCATCCGACTTTGAT
GTGGACAGTTTATCCATACTTGACACCCTGGAGGGAGCTAGCGTG
ACCAGCGGGGCAACGTCAGCCGAGACTAACTCTTACTTCGCAAAG
AGTATGGAGTTTCTGGCGCGACCGGTGCCTGCGCCTCGAACAGTA
TTCAGGAACCCTCCACATCCCGCTCCGCGCACAAGAACACCGTCA
CTTGCACCCAGCAGGGCCTGCTCGAGAACCAGCCTAGTTTCCACC
CCGCCAGGCGTGAATAGGGTGATCACTAGAGAGGAGCTCGAGGCG
CTTACCCCGTCACGCACTCCTAGCAGGTCGGTCTCGAGAACCAGC
CTGGTCTCCAACCCGCCAGGCGTAAATAGGGTGATTACAAGAGAG
GAGTTTGAGGCGTTCGTAGCACAACAACAATGACGGTTTGATGCG
GGTGCA

Accordingly, preferably NSP3 is encoded by a nucleotide sequence as substantially as set out in SEQ ID No: 222, or a variant or fragment thereof.

Thus, the RNA construct may comprise SEQ ID No: 223, as follows:

[SEQ ID No: 223]
GCACCCUCAUAUCAUGUGGUGCGAGGGGAUAUUGCCACGGCCACC
GAAGGAGUGAUUAUAAAUGCUGCUAACAGCAAAGGACAACCUGGC
GGAGGGGUGUGCGGAGCGCUGUAUAAGAAAUUCCCGGAAAGCUUC
GAUUUACAGCCGAUCGAAGUAGGAAAAGCGCGACUGGUCAAAGGU
GCAGCUAAACAUAUCAUUCAUGCCGUAGGACCAAACUUCAACAAA
GUUUCGGAGGUUGAAGGUGACAAACAGUUGGCAGAGGCUUAUGAG
UCCAUCGCUAAGAUUGUCAACGAUAACAAUUACAAGUCAGUAGCG
AUUCCACUGUUGUCCACCGGCAUCUUUUCCGGGAACAAAGAUCGA
CUAACCCAAUCAUUGAACCAUUUGCUGACAGCUUUAGACACCACU
GAUGCAGAUGUAGCCAUAUACUGCAGGGACAAGAAAUGGGAAAUG
ACUCUCAAGGAAGCAGUGGCUAGGAGAGAAGCAGUGGAGGAGAUA
UGCAUAUCCGACGACUCUUCAGUGACAGAACCUGAUGCAGAGCUG
GUGAGGGUGCAUCCGAAGAGUUCUUUGGCUGGAAGGAAGGGCUAC
AGCACAAGCGAUGGCAAAACUUUCUCAUAUUUGGAAGGGACCAAG
UUUCACCAGGCGGCCAAGGAUAUAGCAGAAAUUAAUGCCAUGUGG
CCCGUUGCAACGGAGGCCAAUGAGCAGGUAUGCAUGUAUAUCCUC
GGAGAAAGCAUGAGCAGUAUUAGGUCGAAAUGCCCCGUCGAAGAG
UCGGAAGCCUCCACACCACCUAGCACGCUGCCUUGCUUGUGCAUC
CAUGCCAUGACUCCAGAAAGAGUACAGCGCCUAAAAGCCUCACGU
CCAGAACAAAUUACUGUGUGCUCAUCCUUUCCAUUGCCGAAGUAU
AGAAUCACUGGUGUGCAGAAGAUCCAAUGCUCCCAGCCUAUAUUG
UUCUCACCGAAAGUGCCUGCGUAUAUUCAUCCAAGGAAGUAUCUC
GUGGAAACACCACCGGUAGACGAGACUCCGGAGCCAUCGGCAGAG
AACCAAUCCACAGAGGGGACACCUGAACAACCACCACUUAUAACC
GAGGAUGAGACCAGGACUAGAACGCCUGAGCCGAUCAUCAUCGAA
GAGGAAGAAGAGGAUAGCAUAAGUUUGCUGUCAGAUGGCCCGACC
CACCAGGUGCUGCAAGUCGAGGCAGACAUUCACGGGCCGCCCUCU
GUAUCUAGCUCAUCCUGGUCCAUUCCUCAUGCAUCCGACUUUGAU
GUGGACAGUUUAUCCAUACUUGACACCCUGGAGGGAGCUAGCGUG
ACCAGCGGGGCAACGUCAGCCGAGACUAACUCUUACUUCGCAAAG
AGUAUGGAGUUUCUGGCGCGACCGGUGCCUGCGCCUCGAACAGUA
UUCAGGAACCCUCCACAUCCCGCUCCGCGCACAAGAACACCGUCA
CUUGCACCCAGCAGGGCCUGCUCGAGAACCAGCCUAGUUUCCACC
CCGCCAGGCGUGAAUAGGGUGAUCACUAGAGAGGAGCUCGAGGCG
CUUACCCCGUCACGCACUCCUAGCAGGUCGGUCUCGAGAACCAGC
CUGGUCUCCAACCCGCCAGGCGUAAAUAGGGUGAUUACAAGAGAG
GAGUUUGAGGCGUUCGUAGCACAACAACAAUGACGGUUUGAUGCG
GGUGCA

Accordingly, therefore, preferably the RNA construct comprises an RNA nucleotide sequence substantially as set out as SEQ ID No: 223 or a variant or fragment thereof.

In one embodiment, NSP4 is provided herein as SEQ ID No: 224, as follows:

[SEQ ID No: 224]
YIFSSDTGQGHLQQKSVRQTVLSEVVLERTELEISYAPRLDQEKE
ELLRKKLQLNPTPANRSRYQSRKVENMKAITARRILQGLGHYLKA
EGKVECYRTLHPVPLYSSSVNRAFSSPKVAVEACNAMLKENFPTV
ASYCIIPEYDAYLDMVDGASCCLDTASFCPAKLRSFPKKHSYLEP
TIRSAVPSAIQNTLQNVLAAATKRNCNVTQMRELPVLDSAAFNVE
CFKKYACNNEYWETFKENPIRLTEENVVNYITKLKGPKAAALFAK
THNLNMLQDIPMDRFVMDLKRDVKVTPGTKHTEERPKVQVIQAAD
PLATAYLCGIHRELVRRLNAVLLPNIHTLFDMSAEDFDAIIAEH
FQPGDCVLETDIASFDKSEDDAMALTALMILEDLGVDAELLTLIE
AAFGEISSIHLPTKTKFKFGAMMKSGMFLTLFVNTVINIVIASRV
LRERLTGSPCAAFIGDDNIVKGVKSDKLMADRCATWLNMEVKIID
AVVGEKAPYFCGGFILCDSVTGTACRVADPLKRLFKLGKPLAADD
EHDDDRRRALHEESTRWNRVGILSELCKAVESRYETVGTSIIVMA
MTTLASSVKSFSYLRGAPITLYG

Accordingly, preferably NSP4 comprises an amino acid sequence as substantially as set out in SEQ ID No: 224, or a biologically active variant or fragment thereof.

In one embodiment, NSP4 is encoded by a nucleotide sequence a defined in SEQ ID No: 225, as follows:

[SEQ ID No: 225]
TACATCTTTTCCTCCGACACCGGTCAAGGGCATTTACAACAAAAA
TCAGTAAGGCAAACGGTGCTATCCGAAGTGGTGTTGGAGAGGACC
GAATTGGAGATTTCGTATGCCCCGCGCCTCGACCAAGAAAAAGAA
GAATTACTACGCAAGAAATTACAGTTAAATCCCACACCTGCTAAC
AGAAGCAGATACCAGTCCAGGAAGGTGGAGAACATGAAAGCCATA
ACAGCTAGACGTATTCTGCAAGGCCTAGGGCATTATTTGAAGGCA
GAAGGAAAAGTGGAGTGCTACCGAACCCTGCATCCTGTTCCTTTG
TATTCATCTAGTGTGAACCGTGCCTTTTCAAGCCCCAAGGTCGCA
GTGGAAGCCTGTAACGCCATGTTGAAAGAGAACTTTCCGACTGTG
GCTTCTTACTGTATTATTCCAGAGTACGATGCCTATTTGGACATG
GTTGACGGAGCTTCATGCTGCTTAGACACTGCCAGTTTTTGCCCT
GCAAAGCTGCGCAGCTTTCCAAAGAAACACTCCTATTTGGAACCC
ACAATACGATCGGCAGTGCCTTCAGCGATCCAGAACACGCTCCAG
AACGTCCTGGCAGCTGCCACAAAAAGAAATTGCAATGTCACGCAA
ATGAGAGAATTGCCCGTATTGGATTCGGCGGCCTTTAATGTGGAA
TGCTTCAAGAAATATGCGTGTAATAATGAATATTGGGAAACGTTT
AAAGAAAACCCCATCAGGCTTACTGAAGAAAACGTGGTAAATTAC
ATTACCAAATTAAAAGGACCAAAAGCTGCTGCTCTTTTTGCGAAG
ACACATAATTTGAATATGTTGCAGGACATACCAATGGACAGGTTT
GTAATGGACTTAAAGAGAGACGTGAAAGTGACTCCAGGAACAAAA
CATACTGAAGAACGGCCCAAGGTACAGGTGATCCAGGCTGCCGAT
CCGCTAGCAACAGCGTATCTGTGCGGAATCCACCGAGAGCTGGTT
AGGAGATTAAATGCGGTCCTGCTTCCGAACATTCATACACTGTTT
GATATGTCGGCTGAAGACTTTGACGCTATTATAGCCGAGCACTTC
CAGCCTGGGGATTGTGTTCTGGAAACTGACATCGCGTCGTTTGAT
AAAAGTGAGGACGACGCCATGGCTCTGACCGCGTTAATGATTCTG
GAAGACTTAGGTGTGGACGCAGAGCTGTTGACGCTGATTGAGGCG
GCTTTCGGCGAAATTTCATCAATACATTTGCCCACTAAAACTAAA
TTTAAATTCGGAGCCATGATGAAATCTGGAATGTTCCTCACACTG
TTTGTGAACACAGTCATTAACATTGTAATCGCAAGCAGAGTGTTG
AGAGAACGGCTAACCGGATCACCATGTGCAGCATTCATTGGAGAT
GACAATATCGTGAAAGGAGTCAAATCGGACAAATTAATGGCAGAC
AGGTGCGCCACCTGGTTGAATATGGAAGTCAAGATTATAGATGCT
GTGGTGGGCGAGAAAGCGCCTTATTTCTGTGGAGGGTTTATTTTG
TGTGACTCCGTGACCGGCACAGCGTGCCGTGTGGCAGACCCCCTA
AAAAGGCTGTTTAAGCTTGGCAAACCTCTGGCAGCAGACGATGAA
CATGATGATGACAGGAGAAGGGCATTGCATGAAGAGTCAACACGC
TGGAACCGAGTGGGTATTCTTTCAGAGCTGTGCAAGGCAGTAGAA
TCAAGGTATGAAACCGTAGGAACTTCCATCATAGTTATGGCCATG
ACTACTCTAGCTAGCAGTGTTAAATCATTCAGCTACCTGAGAGGG
GCCCCTATAACTCTCTACGGC

Accordingly, preferably NSP4 is encoded by a nucleotide sequence as substantially as so set out in SEQ ID No: 225, or a variant or fragment thereof.

Thus, the RNA construct may comprise SEQ ID No: 226, as follows:

[SEQ ID No: 226]
UACAUCUUUUCCUCCGACACCGGUCAAGGGCAUUUACAACAAAAA
UCAGUAAGGCAAACGGUGCUAUCCGAAGUGGUGUUGGAGAGGACC
GAAUUGGAGAUUUCGUAUGCCCCGCGCCUCGACCAAGAAAAAGAA
GAAUUACUACGCAAGAAAUUACAGUUAAAUCCCACACCUGCUAAC
AGAAGCAGAUACCAGUCCAGGAAGGUGGAGAACAUGAAAGCCAUA
ACAGCUAGACGUAUUCUGCAAGGCCUAGGGCAUUAUUUGAAGGCA
GAAGGAAAAGUGGAGUGCUACCGAACCCUGCAUCCUGUUCCUUUG
UAUUCAUCUAGUGUGAACCGUGCCUUUUCAAGCCCCAAGGUCGCA
GUGGAAGCCUGUAACGCCAUGUUGAAAGAGAACUUUCCGACUGUG
GCUUCUUACUGUAUUAUUCCAGAGUACGAUGCCUAUUUGGACAUG
GUUGACGGAGCUUCAUGCUGCUUAGACACUGCCAGUUUUUGCCCU
GCAAAGCUGCGCAGCUUUCCAAAGAAACACUCCUAUUUGGAACCC
ACAAUACGAUCGGCAGUGCCUUCAGCGAUCCAGAACACGCUCCAG
AACGUCCUGGCAGCUGCCACAAAAAGAAAUUGCAAUGUCACGCAA
AUGAGAGAAUUGCCCGUAUUGGAUUCGGCGGCCUUUAAUGUGGAA
UGCUUCAAGAAAUAUGCGUGUAAUAAUGAAUAUUGGGAAACGUUU
AAAGAAAACCCCAUCAGGCUUACUGAAGAAAACGUGGUAAAUUAC
AUUACCAAAUUAAAAGGACCAAAAGCUGCUGCUCUUUUUGCGAAG
ACACAUAAUUUGAAUAUGUUGCAGGACAUACCAAUGGACAGGUUU
GUAAUGGACUUAAAGAGAGACGUGAAAGUGACUCCAGGAACAAAA
CAUACUGAAGAACGGCCCAAGGUACAGGUGAUCCAGGCUGCCGAU
CCGCUAGCAACAGCGUAUCUGUGCGGAAUCCACCGAGAGCUGGUU
AGGAGAUUAAAUGCGGUCCUGCUUCCGAACAUUCAUACACUGUUU
GAUAUGUCGGCUGAAGACUUUGACGCUAUUAUAGCCGAGCACUUC
CAGCCUGGGGAUUGUGUUCUGGAAACUGACAUCGCGUCGUUUGAU
AAAAGUGAGGACGACGCCAUGGCUCUGACCGCGUUAAUGAUUCUG
GAAGACUUAGGUGUGGACGCAGAGCUGUUGACGCUGAUUGAGGCG
GCUUUCGGCGAAAUUUCAUCAAUACAUUUGCCCACUAAAACUAAA
UUUAAAUUCGGAGCCAUGAUGAAAUCUGGAAUGUUCCUCACACUG
UUUGUGAACACAGUCAUUAACAUUGUAAUCGCAAGCAGAGUGUUG
AGAGAACGGCUAACCGGAUCACCAUGUGCAGCAUUCAUUGGAGAU
GACAAUAUCGUGAAAGGAGUCAAAUCGGACAAAUUAAUGGCAGAC
AGGUGCGCCACCUGGUUGAAUAUGGAAGUCAAGAUUAUAGAUGCU
GUGGUGGGCGAGAAAGCGCCUUAUUUCUGUGGAGGGUUUAUUUUG
UGUGACUCCGUGACCGGCACAGCGUGCCGUGUGGCAGACCCCCUA
AAAAGGCUGUUUAAGCUUGGCAAACCUCUGGCAGCAGACGAUGAA
CAUGAUGAUGACAGGAGAAGGGCAUUGCAUGAAGAGUCAACACGC
UGGAACCGAGUGGGUAUUCUUUCAGAGCUGUGCAAGGCAGUAGAA
UCAAGGUAUGAAACCGUAGGAACUUCCAUCAUAGUUAUGGCCAUG
ACUACUCUAGCUAGCAGUGUUAAAUCAUUCAGCUACCUGAGAGGG
GCCCCUAUAACUCUCUACGGC

Accordingly, therefore, preferably the RNA construct comprises an RNA nucleotide sequence substantially as set out as SEQ ID No: 226, or a variant or fragment thereof.

Preferably, together with proteins present in a host cell, the non-structural proteins encoded by the RNA construct of the invention form an enzyme complex (i.e. replicase) that is required for genome replication and transcription of the sequences encoding the at least one therapeutic biomolecule and the at least one innate modulatory protein. For example, the one or more non-structural protein may encode a polymerase to enable the construct to amplify the nucleotide sequences encoding the at least one peptide or protein of interest (i.e. therapeutic biomolecule) and the at least one innate modulatory protein.

The host cell may be a eukaryotic or prokaryotic host cell. Preferably, the host cell is a eukaryotic host cell. More preferably, the host cell is a mammalian host cell.

The RNA construct may further comprise a promoter disposed 5′ of the at least one non-structural protein, such that the promoter is operably linked to the sequence encoding the at least one non-structural protein and enables expression of the at least one non-structural protein in a host cell.

Preferably, the RNA construct comprises a 5′ UTR conserved sequence element, which may be referred to herein as SEQ ID No: 227, as follows:

[SEQ ID No: 227]
AUGGGCGGCGCAUGAGAGAAGCCCAGACCAAUUACCUACCCAAA

Accordingly, preferably the UTR is disposed 5′ of the at least one non-structural protein and comprises a nucleotide sequence substantially as set out in SEQ ID No: 227, or a fragment or variant thereof.

Preferably, the RNA construct comprises a 3′ UTR conserved sequence element, which may be referred to herein as SEQ ID No: 228, as follows:

[SEQ ID No: 228]
AAUUGGCAAGCUGCUUACAUAGAACUCGCGGCGAUUGGCAUGCCG
CCUUAAAAUUUUUAUUUUAUUUUUUUUUCUUUUCCGAAUCGGAUU
UUGUUUUUAAUAUUUCAAAAAAAAAAAAAAAAAAAAAAAAAAAAA
AAAAAA

Accordingly, preferably the 3′ UTR is disposed 3′ of the at least one non-structural protein and comprises a nucleotide sequence substantially as set out in SEQ ID No: 228, or a fragment or variant thereof.

Preferably, the RNA construct comprises a polyA tail. Preferably, the polyA tail is disposed at the 3′ end of the construct. The poly A tail may comprise at least 35 nt, or at least 40 nt, or at least 45 nt, or at least 50 nt, wherein each nt is an adenine. In another embodiment, the polyA tail may comprise at least 55 nt or at least 60 nt, wherein each nt is an adenine. In yet another embodiment, the polyA tail may comprise at least 60 adenines, followed by one or more non-adenine nucleotides (i.e. G, C or T, preferably guanine), and then another at least 35 nt, or at least 40 nt, or at least 45 nt, or at least 50 nt, or at least 55 nt, or at least 60 nt, wherein each nt is an adenine.

The RNA construct may further comprise a 5′ cap. In the context of the present invention, the term “5′-cap” includes a 5′-cap analog that resembles the RNA cap structure and is modified to possess the ability to stabilize RNA and/or enhance translation of RNA if attached thereto, preferably in vivo and/or in a cell.

An RNA with a 5′-cap may be achieved by in vitro transcription of a DNA template in presence of said 5′-cap, wherein said 5′-cap is co-transcriptionally incorporated into the generated RNA strand, or the RNA may be generated, for example, by in vitro transcription, and the 5′-cap may be attached to the RNA post-transcriptionally using capping enzymes, for example, capping enzymes of vaccinia virus. In capped RNA, the 3′ position of the first base of a (capped) RNA molecule is linked to the 5′ position of the subsequent base of the RNA molecule (“second base”) via a phosphodiester bond.

In one embodiment, the RNA construct comprises, preferably 5′ to 3′, a promoter, a sequence encoding at least one therapeutic biomolecule, a linker sequence, and at least one sequence encoding a non-viral innate modulatory protein. In one embodiment, the RNA construct comprises, preferably 5′ to 3′, a promoter, a sequence encoding at least one non-viral innate modulatory protein, a linker sequence, and a sequence encoding at least one therapeutic biomolecule. The linker may be F-T2a or IRES in either embodiment.

In another embodiment, the RNA construct comprises, preferably 5′ to 3′, a promoter, a sequence encoding at least one non-structural protein, a sub genomic promoter, a sequence encoding at least one therapeutic biomolecule, a linker sequence, and a sequence encoding at least one non-viral innate modulatory protein. In another embodiment, the RNA construct comprises, preferably 5′ to 3′, a promoter, a sequence encoding at least one non-structural protein, a sub genomic promoter, a sequence encoding at least one non-viral innate modulatory protein, a linker sequence, and a sequence encoding at least one therapeutic biomolecule. The linker may be F-T2a or IRES in either embodiment.

In yet another embodiment, the RNA construct comprises, preferably 5′ to 3′, a promoter, a sequence encoding at least one non-structural protein, a sub genomic promoter, a sequence encoding at least one therapeutic biomolecule, a linker sequence, a sequence encoding at least one non-viral innate modulatory protein, and a polyA tail. In yet another embodiment, the RNA construct comprises, preferably 5′ to 3′, a promoter, a sequence encoding at least one non-structural protein, a sub genomic promoter, a sequence encoding at least one non-viral innate modulatory protein, a linker sequence, a sequence encoding at least one therapeutic biomolecule, and a polyA tail. The linker may be F-T2a or IRES in either embodiment.

In another embodiment, the RNA construct comprises, preferably 5′ to 3′, a promoter, a sequence encoding at least one non-structural protein, a first sub genomic promoter, a sequence encoding at least one therapeutic biomolecule, a second sub genomic promoter, a sequence encoding at least one an innate modulatory protein, and a polyA tail. In another embodiment, the RNA construct comprises, preferably 5′ to 3′, a promoter, a sequence encoding at least one non-structural protein, a first sub genomic promoter, a sequence encoding at least innate modulatory protein, a second sub genomic promoter, a sequence encoding at least one therapeutic biomolecule, and a polyA tail.

Most preferably, the RNA construct comprises, 5′ to 3′, a 5′ cap, a promoter, NSP1, NSP2, NSP3v, NSP4, the sub genomic promoter 26S, a sequence encoding a therapeutic biomolecule, a linker sequence, a sequence encoding the non-viral IMP and a polyA tail. Most preferably, the RNA construct comprises, 5′ to 3′, a 5′ cap, a promoter, NSP1, NSP2, NSP3v, NSP4, the sub genomic promoter 26S, a sequence encoding a non-viral IMP, a linker sequence, a sequence encoding a therapeutic biomolecule; and a polyA tail.

In one embodiment, therefore, the RNA construct may comprise a T7 Promoter, 5′UTR, NSP1-4, Sub-Genomic Promoter, GOI (gene of interest is the therapeutic biomolecule), Furin T2A, IMP is IRF1 (codon optimised with the ATG and stop codon—SEQ ID No:5), 3′UTR, and PolyA tail. Therefore, the RNA construct may comprise or consist of SEQ ID No: 229, a GOI, and SEQ ID No: 264, in a single RNA construct. SEQ ID No: 229 and SEQ ID No 264 are as follows:

[SEQ ID No: 229]
UAAUACGACUCACUAUAGAUGGGCGGCGCAUGAGAGAAGCCCAGA
CCAAUUACCUACCCAAAAUGGAGAAAGUUCACGUUGACAUCGAGG
AAGACAGCCCAUUCCUCAGAGCUUUGCAGCGGAGCUUCCCGCAGU
UUGAGGUAGAAGCCAAGCAGGUCACUGAUAAUGACCAUGCUAAUG
CCAGAGCGUUUUCGCAUCUGGCUUCAAAACUGAUCGAAACGGAGG
UGGACCCAUCCGACACGAUCCUUGACAUUGGAAGUGCGCCCGCCC
GCAGAAUGUAUUCUAAGCACAAGUAUCAUUGUAUCUGUCCGAUGA
GAUGUGCGGAAGAUCCGGACAGAUUGUAUAAGUAUGCAACUAAGC
UGAAGAAAAACUGUAAGGAAAUAACUGAUAAGGAAUUGGACAAGA
AAAUGAAGGAGCUGGCCGCCGUCAUGAGCGACCCUGACCUGGAAA
CUGAGACUAUGUGCCUCCACGACGACGAGUCGUGUCGCUACGAAG
GGCAAGUCGCUGUUUACCAGGAUGUAUACGCGGUUGACGGACCGA
CAAGUCUCUAUCACCAAGCCAAUAAGGGAGUUAGAGUCGCCUACU
GGAUAGGCUUUGACACCACCCCUUUUAUGUUUAAGAACUUGGCUG
GAGCAUAUCCAUCAUACUCUACCAACUGGGCCGACGAAACCGUGU
UAACGGCUCGUAACAUAGGCCUAUGCAGCUCUGACGUUAUGGAGC
GGUCACGUAGAGGGAUGUCCAUUCUUAGAAAGAAGUAUUUGAAAC
CAUCCAACAAUGUUCUAUUCUCUGUUGGCUCGACCAUCUACCACG
AGAAGAGGGACUUACUGAGGAGCUGGCACCUGCCGUCUGUAUUUC
ACUUACGUGGCAAGCAAAAUUACACAUGUCGGUGUGAGACUAUAG
UUAGUUGCGACGGGUACGUCGUUAAAAGAAUAGCUAUCAGUCCAG
GCCUGUAUGGGAAGCCUUCAGGCUAUGCUGCUACGAUGCACCGCG
AGGGAUUCUUGUGCUGCAAAGUGACAGACACAUUGAACGGGGAGA
GGGUCUCUUUUCCCGUGUGCACGUAUGUGCCAGCUACAUUGUGUG
ACCAAAUGACUGGCAUACUGGCAACAGAUGUCAGUGCGGACGACG
CGCAAAAACUGCUGGUUGGGCUCAACCAGCGUAUAGUCGUCAACG
GUCGCACCCAGAGAAACACCAAUACCAUGAAAAAUUACCUUUUGC
CCGUAGUGGCCCAGGCAUUUGCUAGGUGGGCAAAGGAAUAUAAGG
AAGAUCAAGAAGAUGAAAGGCCACUAGGACUACGAGAUAGACAGU
UAGUCAUGGGGUGUUGUUGGGCUUUUAGAAGGCACAAGAUAACAU
CUAUUUAUAAGCGCCCGGAUACCCAAACCAUCAUCAAAGUGAACA
GCGAUUUCCACUCAUUCGUGCUGCCCAGGAUAGGCAGUAACACAU
UGGAGAUCGGGCUGAGAACAAGAAUCAGGAAAAUGUUAGAGGAGC
ACAAGGAGCCGUCACCUCUCAUUACCGCCGAGGACGUACAAGAAG
CUAAGUGCGCAGCCGAUGAGGCUAAGGAGGUGCGUGAAGCCGAGG
AGUUGCGCGCAGCUCUACCACCUUUGGCAGCUGAUGUUGAGGAGC
CCACUCUGGAgGCaGAcGUCGACUUGAUGUUACAAGAGGCUGGGG
CCGGCUCAGUGGAGACACCUCGUGGCUUGAUAAAGGUUACCAGCU
ACGAUGGCGAGGACAAGAUCGGCUCUUACGCUGUGCUUUCUCCGC
AGGCUGUACUCAAGAGUGAAAAAUUAUCUUGCAUCCACCCUCUCG
CUGAACAAGUCAUAGUGAUAACACACUCUGGCCGAAAAGGGCGUU
AUGCCGUGGAACCAUACCAUGGUAAAGUAGUGGUGCCAGAGGGAC
AUGCAAUACCCGUCCAGGACUUUCAAGCUCUGAGUGAAAGUGCCA
CCAUUGUGUACAACGAACGUGAGUUCGUAAACAGGUACCUGCACC
AUAUUGCCACACAUGGAGGAGCGCUGAACACUGAUGAAGAAUAUU
ACAAAACUGUCAAGCCCAGCGAGCACGACGGCGAAUACCUGUACG
ACAUCGACAGGAAACAGUGCGUCAAGAAAGAACUAGUCACUGGGC
UAGGGCUCACAGGCGAGCUGGUGGAUCCUCCCUUCCAUGAAUUCG
CCUACGAGAGUCUGAGAACACGACCAGCCGCUCCUUACCAAGUAC
CAACCAUAGGGGUGUAUGGCGUGCCAGGAUCAGGCAAGUCUGGCA
UCAUUAAAAGCGCAGUCACCAAAAAAGAUCUAGUGGUGAGCGCCA
AGAAAGAAAACUGUGCAGAAAUUAUAAGGGACGUCAAGAAAAUGA
AAGGGCUGGACGUCAAUGCCAGAACUGUGGACUCAGUGCUCUUGA
AUGGAUGCAAACACCCCGUAGAGACCCUGUAUAUUGACGAAGCUU
UUGCUUGUCAUGCAGGUACUCUCAGAGCGCUCAUAGCCAUUAUAA
GACCUAAAAAGGCAGUGCUCUGCGGGGAUCCCAAACAGUGCGGUU
UUUUUAACAUGAUGUGCCUGAAAGUGCAUUUUAACCACGAGAUUU
GCACACAAGUCUUCCACAAAAGCAUCUCUCGCCGUUGCACUAAAU
CUGUGACUUCGGUCGUCUCAACCUUGUUUUACGACAAAAAAAUGA
GAACGACGAAUCCGAAAGAGACUAAGAUUGUGAUUGACACUACCG
GCAGUACCAAACCUAAGCAGGACGAUCUCAUUCUCACUUGUUUCA
GAGGGUGGGUGAAGCAGUUGCAAAUAGAUUACAAAGGCAACGAAA
UAAUGACGGCAGCUGCCUCUCAAGGGCUGACCCGUAAAGGUGUGU
AUGCCGUUCGGUACAAGGUGAAUGAAAAUCCUCUGUACGCACCCA
CCUCAGAACAUGUGAACGUCCUACUGACCCGCACGGAGGACCGCA
UCGUGUGGAAAACACUAGCCGGCGACCCAUGGAUAAAAACACUGA
CUGCCAAGUACCCUGGGAAUUUCACUGCCACGAUAGAGGAGUGGC
AAGCAGAGCAUGAUGCCAUCAUGAGGCACAUCUUGGAGAGACCGG
ACCCUACCGACGUCUUCCAGAAUAAGGCAAACGUGUGUUGGGCCA
AGGCUUUAGUGCCGGUGCUGAAGACCGCUGGCAUAGACAUGACCA
CUGAACAAUGGAACACUGUGGAUUAUUUUGAAACGGACAAAGCUC
ACUCAGCAGAGAUAGUAUUGAACCAACUAUGCGUGAGGUUCUUUG
GACUCGAUCUGGACUCCGGUCUAUUUUCUGCACCCACUGUUCCGU
UAUCCAUUAGGAAUAAUCACUGGGAUAACUCCCCGUCGCCUAACA
UGUACGGGCUGAAUAAAGAAGUGGUCCGUCAGCUCUCUCGCAGGU
ACCCACAACUGCCUCGGGCAGUUGCCACUGGAAGAGUCUAUGACA
UGAACACUGGUACACUGCGCAAUUAUGAUCCGCGCAUAAACCUAG
UACCUGUAAACAGAAGACUGCCUCAUGCUUUAGUCCUCCACCAUA
AUGAACACCCACAGAGUGACUUUUCUUCAUUCGUCAGCAAAUUGA
AGGGCAGAACUGUCCUGGUGGUCGGGGAAAAGUUGUCCGUCCCAG
GCAAAAUGGUUGACUGGUUGUCAGACCGGCCUGAGGCUACCUUCA
GAGCUCGGCUGGAUUUAGGCAUCCCAGGUGAUGUGCCCAAAUAUG
ACAUAAUAUUUGUUAAUGUGAGGACCCCAUAUAAAUACCAUCACU
AUCAGCAGUGUGAAGACCAUGCCAUUAAGCUUAGCAUGUUGACCA
AGAAAGCUUGUCUGCAUCUGAAUCCCGGCGGAACCUGUGUCAGCA
UAGGUUAUGGUUACGCUGACAGGGCCAGCGAAAGCAUCAUUGGUG
CUAUAGCGCGGCAGUUCAAGUUUUCCCGGGUAUGCAAACCGAAAU
CCUCACUUGAAGAGACGGAAGUUCUGUUUGUAUUCAUUGGGUACG
AUCGCAAGGCCCGUACGCACAAUUCUUACAAGCUUUCAUCAACCU
UGACCAACAUUUAUACAGGUUCCAGACUCCACGAAGCCGGAUGUG
CACCCUCAUAUCAUGUGGUGCGAGGGGAUAUUGCCACGGCCACCG
AAGGAGUGAUUAUAAAUGCUGCUAACAGCAAAGGACAACCUGGCG
GAGGGGUGUGCGGAGCGCUGUAUAAGAAAUUCCCGGAAAGCUUCG
AUUUACAGCCGAUCGAAGUAGGAAAAGCGCGACUGGUCAAAGGUG
CAGCUAAACAUAUCAUUCAUGCCGUAGGACCAAACUUCAACAAAG
UUUCGGAGGUUGAAGGUGACAAACAGUUGGCAGAGGCUUAUGAGU
CCAUCGCUAAGAUUGUCAACGAUAACAAUUACAAGUCAGUAGCGA
UUCCACUGUUGUCCACCGGCAUCUUUUCCGGGAACAAAGAUCGAC
UAACCCAAUCAUUGAACCAUUUGCUGACAGCUUUAGACACCACUG
AUGCAGAUGUAGCCAUAUACUGCAGGGACAAGAAAUGGGAAAUGA
CUCUCAAGGAAGCAGUGGCUAGGAGAGAAGCAGUGGAGGAGAUAU
GCAUAUCCGACGACUCUUCAGUGACAGAACCUGAUGCAGAGCUGG
UGAGGGUGCAUCCGAAGAGUUCUUUGGCUGGAAGGAAGGGCUACA
GCACAAGCGAUGGCAAAACUUUCUCAUAUUUGGAAGGGACCAAGU
UUCACCAGGCGGCCAAGGAUAUAGCAGAAAUUAAUGCCAUGUGGC
CCGUUGCAACGGAGGCCAAUGAGCAGGUAUGCAUGUAUAUCCUCG
GAGAAAGCAUGAGCAGUAUUAGGUCGAAAUGCCCCGUCGAAGAGU
CGGAAGCCUCCACACCACCUAGCACGCUGCCUUGCUUGUGCAUCC
AUGCCAUGACUCCAGAAAGAGUACAGCGCCUAAAAGCCUCACGUC
CAGAACAAAUUACUGUGUGCUCAUCCUUUCCAUUGCCGAAGUAUA
GAAUCACUGGUGUGCAGAAGAUCCAAUGCUCCCAGCCUAUAUUGU
UCUCACCGAAAGUGCCUGCGUAUAUUCAUCCAAGGAAGUAUCUCG
UGGAAACACCACCGGUAGACGAGACUCCGGAGCCAUCGGCAGAGA
ACCAAUCCACAGAGGGGACACCUGAACAACCACCACUUAUAACCG
AGGAUGAGACCAGGACUAGAACGCCUGAGCCGAUCAUCAUCGAAG
AGGAAGAAGAGGAUAGCAUAAGUUUGCUGUCAGAUGGCCCGACCC
ACCAGGUGCUGCAAGUCGAGGCAGACAUUCACGGGCCGCCCUCUG
UAUCUAGCUCAUCCUGGUCCAUUCCUCAUGCAUCCGACUUUGAUG
UGGACAGUUUAUCCAUACUUGACACCCUGGAGGGAGCUAGCGUGA
CCAGCGGGGCAACGUCAGCCGAGACUAACUCUUACUUCGCAAAGA
GUAUGGAGUUUCUGGCGCGACCGGUGCCUGCGCCUCGAACAGUAU
UCAGGAACCCUCCACAUCCCGCUCCGCGCACAAGAACACCGUCAC
UUGCACCCAGCAGGGCCUGCUCGAGAACCAGCCUAGUUUCCACCC
CGCCAGGCGUGAAUAGGGUGAUCACUAGAGAGGAGCUCGAGGCGC
UUACCCCGUCACGCACUCCUAGCAGGUCGGUCUCGAGAACCAGCC
UGGUCUCCAACCCGCCAGGCGUAAAUAGGGUGAUUACAAGAGAGG
AGUUUGAGGCGUUCGUAGCACAACAACAAUGACGGUUUGAUGCGG
GUGCAUACAUCUUUUCCUCCGACACCGGUCAAGGGCAUUUACAAC
AAAAAUCAGUAAGGCAAACGGUGCUAUCCGAAGUGGUGUUGGAGA
GGACCGAAUUGGAGAUUUCGUAUGCCCCGCGCCUCGACCAAGAAA
AAGAAGAAUUACUACGCAAGAAAUUACAGUUAAAUCCCACACCUG
CUAACAGAAGCAGAUACCAGUCCAGGAAGGUGGAGAACAUGAAAG
CCAUAACAGCUAGACGUAUUCUGCAAGGCCUAGGGCAUUAUUUGA
AGGCAGAAGGAAAAGUGGAGUGCUACCGAACCCUGCAUCCUGUUC
CUUUGUAUUCAUCUAGUGUGAACCGUGCCUUUUCAAGCCCCAAGG
UCGCAGUGGAAGCCUGUAACGCCAUGUUGAAAGAGAACUUUCCGA
CUGUGGCUUCUUACUGUAUUAUUCCAGAGUACGAUGCCUAUUUGG
ACAUGGUUGACGGAGCUUCAUGCUGCUUAGACACUGCCAGUUUUU
GCCCUGCAAAGCUGCGCAGCUUUCCAAAGAAACACUCCUAUUUGG
AACCCACAAUACGAUCGGCAGUGCCUUCAGCGAUCCAGAACACGC
UCCAGAACGUCCUGGCAGCUGCCACAAAAAGAAAUUGCAAUGUCA
CGCAAAUGAGAGAAUUGCCCGUAUUGGAUUCGGCGGCCUUUAAUG
UGGAAUGCUUCAAGAAAUAUGCGUGUAAUAAUGAAUAUUGGGAAA
CGUUUAAAGAAAACCCCAUCAGGCUUACUGAAGAAAACGUGGUAA
AUUACAUUACCAAAUUAAAAGGACCAAAAGCUGCUGCUCUUUUUG
CGAAGACACAUAAUUUGAAUAUGUUGCAGGACAUACCAAUGGACA
GGUUUGUAAUGGACUUAAAGAGAGACGUGAAAGUGACUCCAGGAA
CAAAACAUACUGAAGAACGGCCCAAGGUACAGGUGAUCCAGGCUG
CCGAUCCGCUAGCAACAGCGUAUCUGUGCGGAAUCCACCGAGAGC
UGGUUAGGAGAUUAAAUGCGGUCCUGCUUCCGAACAUUCAUACAC
UGUUUGAUAUGUCGGCUGAAGACUUUGACGCUAUUAUAGCCGAGC
ACUUCCAGCCUGGGGAUUGUGUUCUGGAAACUGACAUCGCGUCGU
UUGAUAAAAGUGAGGACGACGCCAUGGCUCUGACCGCGUUAAUGA
UUCUGGAAGACUUAGGUGUGGACGCAGAGCUGUUGACGCUGAUUG
AGGCGGCUUUCGGCGAAAUUUCAUCAAUACAUUUGCCCACUAAAA
CUAAAUUUAAAUUCGGAGCCAUGAUGAAAUCUGGAAUGUUCCUCA
CACUGUUUGUGAACACAGUCAUUAACAUUGUAAUCGCAAGCAGAG
UGUUGAGAGAACGGCUAACCGGAUCACCAUGUGCAGCAUUCAUUG
GAGAUGACAAUAUCGUGAAAGGAGUCAAAUCGGACAAAUUAAUGG
CAGACAGGUGCGCCACCUGGUUGAAUAUGGAAGUCAAGAUUAUAG
AUGCUGUGGUGGGCGAGAAAGCGCCUUAUUUCUGUGGAGGGUUUA
UUUUGUGUGACUCCGUGACCGGCACAGCGUGCCGUGUGGCAGACC
CCCUAAAAAGGCUGUUUAAGCUUGGCAAACCUCUGGCAGCAGACG
AUGAACAUGAUGAUGACAGGAGAAGGGCAUUGCAUGAAGAGUCAA
CACGCUGGAACCGAGUGGGUAUUCUUUCAGAGCUGUGCAAGGCAG
UAGAAUCAAGGUAUGAAACCGUAGGAACUUCCAUCAUAGUUAUGG
CCAUGACUACUCUAGCUAGCAGUGUUAAAUCAUUCAGCUACCUGA
GAGGGGCCCCUAUAACUCUCUACGGCUAACCUGAAUGGACUACGA
CAUAGUCUAGUCCGCCAAGUCUAGCAU
[SEQ ID No: 264]
-----GOI----------------------
CGGAGACGGCGCAGAAGAAGAGGAUCUGGCGAAGGCAGAGGCAGC
CUGCUuACAUGuGGcGACGUGGAAGAGAACCCCGGACCUAUGGGC
GAUAGCAGCCCCGAUACCUUUUCCGAUGGCCUGAGCAGCAGCACC
CUGCCUGAUGAUCACAGCAGCUACACCGUGCCUGGCUACAUGCAG
GACCUGGAAGUGGAACAGGCCCUGACACCAGCUCUGAGCCCUUGU
GCUGUGUCCAGCACACUGCCCGAUUGGCACAUCCCUGUGGAAGUG
GUGCCUGACAGCACCAGCGACCUGUACAACUUCCAAGUGUCCCCU
AUGCCUAGCACCUCCGAGGCCACCACCGAUGAGGAUGAAGAGGGA
AAGCUGCCCGAGGACAUCAUGAAGCUGCUGGAACAGAGCGAGUGG
CAGCCCACCAAUGUGGAUGGCAAGGGCUACCUGCUGAACGAGCCU
GGCGUUCAGCCUACAAGCGUGUACGGCGACUUCAGCUGCAAAGAG
GAACCCGAGAUCGAUAGCCCUGGCGGCGAUAUCGGACUGAGCCUG
CAGAGAGUGUUCACCGACCUGAAGAACAUGGACGCCACCUGGCUG
GACAGCCUGCUGACACCUGUUAGACUGCCCUCUAUCCAGGCUAUC
CCCUGCGCUCCUUGAGCGGCCGCGAAUUGGCAAGCUGCUUACAUA
GAACUCGCGGCGAUUGGCAUGCCGCCUUAAAAUUUUUAUUUUAUU
UUUCUUUUCUUUUCCGAAUCGGAUUUUGUUUUUAAUAUUUCAAAA
AAAAAAAAAAAAAAAAAAAAAAAAAAAAAAA

Accordingly, preferably the RNA construct comprises a nucleotide sequence substantially as set out above, comprising or consisting of SEQ ID No: 229, a GOI, and SEQ ID No: 264, or a fragment or variant thereof.

In a second aspect of the invention, there is provided a nucleic acid sequence encoding the RNA construct of the first aspect.

In one embodiment, therefore, the nucleic acid sequence may comprise a T7 Promoter, 5′UTR, NSP1-4, Sub-Genomic Promoter, GOI (gene of interest is the therapeutic biomolecule), Furin T2A, IMP is IRF1 (codon optimised with the ATG and stop codon−SEQ ID No:4), 3′UTR, and PolyA tail. In one embodiment, therefore, the nucleic acid sequence may comprise or consist of SEQ ID No: 230, a GOI, and SEQ ID No: 265. SEQ ID No: 230 and SEQ ID No: 265 are as follows:

[SEQ ID No: 230]
TAATACGACTCACTATAGATGGGCGGCGCATGAGAGAAGCCCAGA
CCAATTACCTACCCAAAATGGAGAAAGTTCACGTTGACATCGAGG
AAGACAGCCCATTCCTCAGAGCTTTGCAGCGGAGCTTCCCGCAGT
TTGAGGTAGAAGCCAAGCAGGTCACTGATAATGACCATGCTAATG
CCAGAGCGTTTTCGCATCTGGCTTCAAAACTGATCGAAACGGAGG
TGGACCCATCCGACACGATCCTTGACATTGGAAGTGCGCCCGCCC
GCAGAATGTATTCTAAGCACAAGTATCATTGTATCTGTCCGATGA
GATGTGCGGAAGATCCGGACAGATTGTATAAGTATGCAACTAAGC
TGAAGAAAAACTGTAAGGAAATAACTGATAAGGAATTGGACAAGA
AAATGAAGGAGCTGGCCGCCGTCATGAGCGACCCTGACCTGGAAA
CTGAGACTATGTGCCTCCACGACGACGAGTCGTGTCGCTACGAAG
GGCAAGTCGCTGTTTACCAGGATGTATACGCGGTTGACGGACCGA
CAAGTCTCTATCACCAAGCCAATAAGGGAGTTAGAGTCGCCTACT
GGATAGGCTTTGACACCACCCCTTTTATGTTTAAGAACTTGGCTG
GAGCATATCCATCATACTCTACCAACTGGGCCGACGAAACCGTGT
TAACGGCTCGTAACATAGGCCTATGCAGCTCTGACGTTATGGAGC
GGTCACGTAGAGGGATGTCCATTCTTAGAAAGAAGTATTTGAAAC
CATCCAACAATGTTCTATTCTCTGTTGGCTCGACCATCTACCACG
AGAAGAGGGACTTACTGAGGAGCTGGCACCTGCCGTCTGTATTTC
ACTTACGTGGCAAGCAAAATTACACATGTCGGTGTGAGACTATAG
TTAGTTGCGACGGGTACGTCGTTAAAAGAATAGCTATCAGTCCAG
GCCTGTATGGGAAGCCTTCAGGCTATGCTGCTACGATGCACCGCG
AGGGATTCTTGTGCTGCAAAGTGACAGACACATTGAACGGGGAGA
GGGTCTCTTTTCCCGTGTGCACGTATGTGCCAGCTACATTGTGTG
ACCAAATGACTGGCATACTGGCAACAGATGTCAGTGCGGACGACG
CGCAAAAACTGCTGGTTGGGCTCAACCAGCGTATAGTCGTCAACG
GTCGCACCCAGAGAAACACCAATACCATGAAAAATTACCTTTTGC
CCGTAGTGGCCCAGGCATTTGCTAGGTGGGCAAAGGAATATAAGG
AAGATCAAGAAGATGAAAGGCCACTAGGACTACGAGATAGACAGT
TAGTCATGGGGTGTTGTTGGGCTTTTAGAAGGCACAAGATAACAT
CTATTTATAAGCGCCCGGATACCCAAACCATCATCAAAGTGAACA
GCGATTTCCACTCATTCGTGCTGCCCAGGATAGGCAGTAACACAT
TGGAGATCGGGCTGAGAACAAGAATCAGGAAAATGTTAGAGGAGC
ACAAGGAGCCGTCACCTCTCATTACCGCCGAGGACGTACAAGAAG
CTAAGTGCGCAGCCGATGAGGCTAAGGAGGTGCGTGAAGCCGAGG
AGTTGCGCGCAGCTCTACCACCTTTGGCAGCTGATGTTGAGGAGC
CCACTCTGGAgGCaGAcGTCGACTTGATGTTACAAGAGGCTGGGG
CCGGCTCAGTGGAGACACCTCGTGGCTTGATAAAGGTTACCAGCT
ACGATGGCGAGGACAAGATCGGCTCTTACGCTGTGCTTTCTCCGC
AGGCTGTACTCAAGAGTGAAAAATTATCTTGCATCCACCCTCTCG
CTGAACAAGTCATAGTGATAACACACTCTGGCCGAAAAGGGCGTT
ATGCCGTGGAACCATACCATGGTAAAGTAGTGGTGCCAGAGGGAC
ATGCAATACCCGTCCAGGACTTTCAAGCTCTGAGTGAAAGTGCCA
CCATTGTGTACAACGAACGTGAGTTCGTAAACAGGTACCTGCACC
ATATTGCCACACATGGAGGAGCGCTGAACACTGATGAAGAATATT
ACAAAACTGTCAAGCCCAGCGAGCACGACGGCGAATACCTGTACG
ACATCGACAGGAAACAGTGCGTCAAGAAAGAACTAGTCACTGGGC
TAGGGCTCACAGGCGAGCTGGTGGATCCTCCCTTCCATGAATTCG
CCTACGAGAGTCTGAGAACACGACCAGCCGCTCCTTACCAAGTAC
CAACCATAGGGGTGTATGGCGTGCCAGGATCAGGCAAGTCTGGCA
TCATTAAAAGCGCAGTCACCAAAAAAGATCTAGTGGTGAGCGCCA
AGAAAGAAAACTGTGCAGAAATTATAAGGGACGTCAAGAAAATGA
AAGGGCTGGACGTCAATGCCAGAACTGTGGACTCAGTGCTCTTGA
ATGGATGCAAACACCCCGTAGAGACCCTGTATATTGACGAAGCTT
TTGCTTGTCATGCAGGTACTCTCAGAGCGCTCATAGCCATTATAA
GACCTAAAAAGGCAGTGCTCTGCGGGGATCCCAAACAGTGCGGTT
TTTTTAACATGATGTGCCTGAAAGTGCATTTTAACCACGAGATTT
GCACACAAGTCTTCCACAAAAGCATCTCTCGCCGTTGCACTAAAT
CTGTGACTTCGGTCGTCTCAACCTTGTTTTACGACAAAAAAATGA
GAACGACGAATCCGAAAGAGACTAAGATTGTGATTGACACTACCG
GCAGTACCAAACCTAAGCAGGACGATCTCATTCTCACTTGTTTCA
GAGGGTGGGTGAAGCAGTTGCAAATAGATTACAAAGGCAACGAAA
TAATGACGGCAGCTGCCTCTCAAGGGCTGACCCGTAAAGGTGTGT
ATGCCGTTCGGTACAAGGTGAATGAAAATCCTCTGTACGCACCCA
CCTCAGAACATGTGAACGTCCTACTGACCCGCACGGAGGACCGCA
TCGTGTGGAAAACACTAGCCGGCGACCCATGGATAAAAACACTGA
CTGCCAAGTACCCTGGGAATTTCACTGCCACGATAGAGGAGTGGC
AAGCAGAGCATGATGCCATCATGAGGCACATCTTGGAGAGACCGG
ACCCTACCGACGTCTTCCAGAATAAGGCAAACGTGTGTTGGGCCA
AGGCTTTAGTGCCGGTGCTGAAGACCGCTGGCATAGACATGACCA
CTGAACAATGGAACACTGTGGATTATTTTGAAACGGACAAAGCTC
ACTCAGCAGAGATAGTATTGAACCAACTATGCGTGAGGTTCTTTG
GACTCGATCTGGACTCCGGTCTATTTTCTGCACCCACTGTTCCGT
TATCCATTAGGAATAATCACTGGGATAACTCCCCGTCGCCTAACA
TGTACGGGCTGAATAAAGAAGTGGTCCGTCAGCTCTCTCGCAGGT
ACCCACAACTGCCTCGGGCAGTTGCCACTGGAAGAGTCTATGACA
TGAACACTGGTACACTGCGCAATTATGATCCGCGCATAAACCTAG
TACCTGTAAACAGAAGACTGCCTCATGCTTTAGTCCTCCACCATA
ATGAACACCCACAGAGTGACTTTTCTTCATTCGTCAGCAAATTGA
AGGGCAGAACTGTCCTGGTGGTCGGGGAAAAGTTGTCCGTCCCAG
GCAAAATGGTTGACTGGTTGTCAGACCGGCCTGAGGCTACCTTCA
GAGCTCGGCTGGATTTAGGCATCCCAGGTGATGTGCCCAAATATG
ACATAATATTTGTTAATGTGAGGACCCCATATAAATACCATCACT
ATCAGCAGTGTGAAGACCATGCCATTAAGCTTAGCATGTTGACCA
AGAAAGCTTGTCTGCATCTGAATCCCGGCGGAACCTGTGTCAGCA
TAGGTTATGGTTACGCTGACAGGGCCAGCGAAAGCATCATTGGTG
CTATAGCGCGGCAGTTCAAGTTTTCCCGGGTATGCAAACCGAAAT
CCTCACTTGAAGAGACGGAAGTTCTGTTTGTATTCATTGGGTACG
ATCGCAAGGCCCGTACGCACAATTCTTACAAGCTTTCATCAACCT
TGACCAACATTTATACAGGTTCCAGACTCCACGAAGCCGGATGTG
CACCCTCATATCATGTGGTGCGAGGGGATATTGCCACGGCCACCG
AAGGAGTGATTATAAATGCTGCTAACAGCAAAGGACAACCTGGCG
GAGGGGTGTGCGGAGCGCTGTATAAGAAATTCCCGGAAAGCTTCG
ATTTACAGCCGATCGAAGTAGGAAAAGCGCGACTGGTCAAAGGTG
CAGCTAAACATATCATTCATGCCGTAGGACCAAACTTCAACAAAG
TTTCGGAGGTTGAAGGTGACAAACAGTTGGCAGAGGCTTATGAGT
CCATCGCTAAGATTGTCAACGATAACAATTACAAGTCAGTAGCGA
TTCCACTGTTGTCCACCGGCATCTTTTCCGGGAACAAAGATCGAC
TAACCCAATCATTGAACCATTTGCTGACAGCTTTAGACACCACTG
ATGCAGATGTAGCCATATACTGCAGGGACAAGAAATGGGAAATGA
CTCTCAAGGAAGCAGTGGCTAGGAGAGAAGCAGTGGAGGAGATAT
GCATATCCGACGACTCTTCAGTGACAGAACCTGATGCAGAGCTGG
TGAGGGTGCATCCGAAGAGTTCTTTGGCTGGAAGGAAGGGCTACA
GCACAAGCGATGGCAAAACTTTCTCATATTTGGAAGGGACCAAGT
TTCACCAGGCGGCCAAGGATATAGCAGAAATTAATGCCATGTGGC
CCGTTGCAACGGAGGCCAATGAGCAGGTATGCATGTATATCCTCG
GAGAAAGCATGAGCAGTATTAGGTCGAAATGCCCCGTCGAAGAGT
CGGAAGCCTCCACACCACCTAGCACGCTGCCTTGCTTGTGCATCC
ATGCCATGACTCCAGAAAGAGTACAGCGCCTAAAAGCCTCACGTC
CAGAACAAATTACTGTGTGCTCATCCTTTCCATTGCCGAAGTATA
GAATCACTGGTGTGCAGAAGATCCAATGCTCCCAGCCTATATTGT
TCTCACCGAAAGTGCCTGCGTATATTCATCCAAGGAAGTATCTCG
TGGAAACACCACCGGTAGACGAGACTCCGGAGCCATCGGCAGAGA
ACCAATCCACAGAGGGGACACCTGAACAACCACCACTTATAACCG
AGGATGAGACCAGGACTAGAACGCCTGAGCCGATCATCATCGAAG
AGGAAGAAGAGGATAGCATAAGTTTGCTGTCAGATGGCCCGACCC
ACCAGGTGCTGCAAGTCGAGGCAGACATTCACGGGCCGCCCTCTG
TATCTAGCTCATCCTGGTCCATTCCTCATGCATCCGACTTTGATG
TGGACAGTTTATCCATACTTGACACCCTGGAGGGAGCTAGCGTGA
CCAGCGGGGCAACGTCAGCCGAGACTAACTCTTACTTCGCAAAGA
GTATGGAGTTTCTGGCGCGACCGGTGCCTGCGCCTCGAACAGTAT
TCAGGAACCCTCCACATCCCGCTCCGCGCACAAGAACACCGTCAC
TTGCACCCAGCAGGGCCTGCTCGAGAACCAGCCTAGTTTCCACCC
CGCCAGGCGTGAATAGGGTGATCACTAGAGAGGAGCTCGAGGCGC
TTACCCCGTCACGCACTCCTAGCAGGTCGGTCTCGAGAACCAGCC
TGGTCTCCAACCCGCCAGGCGTAAATAGGGTGATTACAAGAGAGG
AGTTTGAGGCGTTCGTAGCACAACAACAATGACGGTTTGATGCGG
GTGCATACATCTTTTCCTCCGACACCGGTCAAGGGCATTTACAAC
AAAAATCAGTAAGGCAAACGGTGCTATCCGAAGTGGTGTTGGAGA
GGACCGAATTGGAGATTTCGTATGCCCCGCGCCTCGACCAAGAAA
AAGAAGAATTACTACGCAAGAAATTACAGTTAAATCCCACACCTG
CTAACAGAAGCAGATACCAGTCCAGGAAGGTGGAGAACATGAAAG
CCATAACAGCTAGACGTATTCTGCAAGGCCTAGGGCATTATTTGA
AGGCAGAAGGAAAAGTGGAGTGCTACCGAACCCTGCATCCTGTTC
CTTTGTATTCATCTAGTGTGAACCGTGCCTTTTCAAGCCCCAAGG
TCGCAGTGGAAGCCTGTAACGCCATGTTGAAAGAGAACTTTCCGA
CTGTGGCTTCTTACTGTATTATTCCAGAGTACGATGCCTATTTGG
ACATGGTTGACGGAGCTTCATGCTGCTTAGACACTGCCAGTTTTT
GCCCTGCAAAGCTGCGCAGCTTTCCAAAGAAACACTCCTATTTGG
AACCCACAATACGATCGGCAGTGCCTTCAGCGATCCAGAACACGC
TCCAGAACGTCCTGGCAGCTGCCACAAAAAGAAATTGCAATGTCA
CGCAAATGAGAGAATTGCCCGTATTGGATTCGGCGGCCTTTAATG
TGGAATGCTTCAAGAAATATGCGTGTAATAATGAATATTGGGAAA
CGTTTAAAGAAAACCCCATCAGGCTTACTGAAGAAAACGTGGTAA
ATTACATTACCAAATTAAAAGGACCAAAAGCTGCTGCTCTTTTTG
CGAAGACACATAATTTGAATATGTTGCAGGACATACCAATGGACA
GGTTTGTAATGGACTTAAAGAGAGACGTGAAAGTGACTCCAGGAA
CAAAACATACTGAAGAACGGCCCAAGGTACAGGTGATCCAGGCTG
CCGATCCGCTAGCAACAGCGTATCTGTGCGGAATCCACCGAGAGC
TGGTTAGGAGATTAAATGCGGTCCTGCTTCCGAACATTCATACAC
TGTTTGATATGTCGGCTGAAGACTTTGACGCTATTATAGCCGAGC
ACTTCCAGCCTGGGGATTGTGTTCTGGAAACTGACATCGCGTCGT
TTGATAAAAGTGAGGACGACGCCATGGCTCTGACCGCGTTAATGA
TTCTGGAAGACTTAGGTGTGGACGCAGAGCTGTTGACGCTGATTG
AGGCGGCTTTCGGCGAAATTTCATCAATACATTTGCCCACTAAAA
CTAAATTTAAATTCGGAGCCATGATGAAATCTGGAATGTTCCTCA
CACTGTTTGTGAACACAGTCATTAACATTGTAATCGCAAGCAGAG
TGTTGAGAGAACGGCTAACCGGATCACCATGTGCAGCATTCATTG
GAGATGACAATATCGTGAAAGGAGTCAAATCGGACAAATTAATGG
CAGACAGGTGCGCCACCTGGTTGAATATGGAAGTCAAGATTATAG
ATGCTGTGGTGGGCGAGAAAGCGCCTTATTTCTGTGGAGGGTTTA
TTTTGTGTGACTCCGTGACCGGCACAGCGTGCCGTGTGGCAGACC
CCCTAAAAAGGCTGTTTAAGCTTGGCAAACCTCTGGCAGCAGACG
ATGAACATGATGATGACAGGAGAAGGGCATTGCATGAAGAGTCAA
CACGCTGGAACCGAGTGGGTATTCTTTCAGAGCTGTGCAAGGCAG
TAGAATCAAGGTATGAAACCGTAGGAACTTCCATCATAGTTATGG
CCATGACTACTCTAGCTAGCAGTGTTAAATCATTCAGCTACCTGA
GAGGGGCCCCTATAACTCTCTACGGCTAACCTGAATGGACTACGA
CATAGTCTAGTCCGCCAAGTCTAGCAT
[SEQ ID No: 265]
---GOI---
CGGAGACGGCGCAGAAGAAGAGGATCTGGCGAAGGCAGAGGCAGC
CTGCTtACATGtGGcGACGTGGAAGAGAACCCCGGACCTATGGGC
GATAGCAGCCCCGATACCTTTTCCGATGGCCTGAGCAGCAGCACC
CTGCCTGATGATCACAGCAGCTACACCGTGCCTGGCTACATGCAG
GACCTGGAAGTGGAACAGGCCCTGACACCAGCTCTGAGCCCTTGT
GCTGTGTCCAGCACACTGCCCGATTGGCACATCCCTGTGGAAGTG
GTGCCTGACAGCACCAGCGACCTGTACAACTTCCAAGTGTCCCCT
ATGCCTAGCACCTCCGAGGCCACCACCGATGAGGATGAAGAGGGA
AAGCTGCCCGAGGACATCATGAAGCTGCTGGAACAGAGCGAGTGG
CAGCCCACCAATGTGGATGGCAAGGGCTACCTGCTGAACGAGCCT
GGCGTTCAGCCTACAAGCGTGTACGGCGACTTCAGCTGCAAAGAG
GAACCCGAGATCGATAGCCCTGGCGGCGATATCGGACTGAGCCTG
CAGAGAGTGTTCACCGACCTGAAGAACATGGACGCCACCTGGCTG
GACAGCCTGCTGACACCTGTTAGACTGCCCTCTATCCAGGCTATC
CCCTGCGCTCCTTGAGCGGCCGCGAATTGGCAAGCTGCTTACATA
GAACTCGCGGCGATTGGCATGCCGCCTTAAAATTTTTATTTTATT
TTTCTTTTCTTTTCCGAATCGGATTTTGTTTTTAATATTTCAAAA
AAAAAAAAAAAAAAAAAAAAAAAAAAAAAAA

Accordingly, preferably the nucleic acid sequence comprises a nucleotide sequence substantially as set out above, comprising or consisting of SEQ ID No: 230, a GOI, and SEQ ID No: 265, or a fragment or variant thereof.

In a third aspect, there is provided an expression cassette comprising a nucleic acid sequence according to the second aspect.

The nucleic acid sequences of the invention are preferably harboured in a recombinant vector, for example a recombinant vector for delivery into a host cell of interest to enable production of the RNA construct.

Accordingly, in a fourth aspect, there is provided a recombinant vector comprising the expression cassette according to the third aspect.

In one embodiment, therefore, the vector may comprise a T7 Promoter, 5′UTR, NSP1-4, Sub-Genomic Promoter, GOI (gene of interest is the therapeutic biomolecule), Furin T2A, IMP is IRF1 (codon optimised with the ATG and stop codon—SEQ ID No: 5), 3′UTR, and PolyA tail. In one embodiment, the vector may comprise the nucleic acid sequence of SEQ ID No: 231, a GOI, and the nucleic acid sequence of SEQ ID No: 266, in a single vector. SEQ ID No: 231 and SEQ ID No: 266 are as follows, where “GOI” represents the position of the therapeutic biomolecule encoding sequence:

[SEQ ID No: 231]
TAATACGACTCACTATAGATGGGCGGCGCATGAGAGAAGCCCAGA
CCAATTACCTACCCAAAATGGAGAAAGTTCACGTTGACATCGAGG
AAGACAGCCCATTCCTCAGAGCTTTGCAGCGGAGCTTCCCGCAGT
TTGAGGTAGAAGCCAAGCAGGTCACTGATAATGACCATGCTAATG
CCAGAGCGTTTTCGCATCTGGCTTCAAAACTGATCGAAACGGAGG
TGGACCCATCCGACACGATCCTTGACATTGGAAGTGCGCCCGCCC
GCAGAATGTATTCTAAGCACAAGTATCATTGTATCTGTCCGATGA
GATGTGCGGAAGATCCGGACAGATTGTATAAGTATGCAACTAAGC
TGAAGAAAAACTGTAAGGAAATAACTGATAAGGAATTGGACAAGA
AAATGAAGGAGCTGGCCGCCGTCATGAGCGACCCTGACCTGGAAA
CTGAGACTATGTGCCTCCACGACGACGAGTCGTGTCGCTACGAAG
GGCAAGTCGCTGTTTACCAGGATGTATACGCGGTTGACGGACCGA
CAAGTCTCTATCACCAAGCCAATAAGGGAGTTAGAGTCGCCTACT
GGATAGGCTTTGACACCACCCCTTTTATGTTTAAGAACTTGGCTG
GAGCATATCCATCATACTCTACCAACTGGGCCGACGAAACCGTGT
TAACGGCTCGTAACATAGGCCTATGCAGCTCTGACGTTATGGAGC
GGTCACGTAGAGGGATGTCCATTCTTAGAAAGAAGTATTTGAAAC
CATCCAACAATGTTCTATTCTCTGTTGGCTCGACCATCTACCACG
AGAAGAGGGACTTACTGAGGAGCTGGCACCTGCCGTCTGTATTTC
ACTTACGTGGCAAGCAAAATTACACATGTCGGTGTGAGACTATAG
TTAGTTGCGACGGGTACGTCGTTAAAAGAATAGCTATCAGTCCAG
GCCTGTATGGGAAGCCTTCAGGCTATGCTGCTACGATGCACCGCG
AGGGATTCTTGTGCTGCAAAGTGACAGACACATTGAACGGGGAGA
GGGTCTCTTTTCCCGTGTGCACGTATGTGCCAGCTACATTGTGTG
ACCAAATGACTGGCATACTGGCAACAGATGTCAGTGCGGACGACG
CGCAAAAACTGCTGGTTGGGCTCAACCAGCGTATAGTCGTCAACG
GTCGCACCCAGAGAAACACCAATACCATGAAAAATTACCTTTTGC
CCGTAGTGGCCCAGGCATTTGCTAGGTGGGCAAAGGAATATAAGG
AAGATCAAGAAGATGAAAGGCCACTAGGACTACGAGATAGACAGT
TAGTCATGGGGTGTTGTTGGGCTTTTAGAAGGCACAAGATAACAT
CTATTTATAAGCGCCCGGATACCCAAACCATCATCAAAGTGAACA
GCGATTTCCACTCATTCGTGCTGCCCAGGATAGGCAGTAACACAT
TGGAGATCGGGCTGAGAACAAGAATCAGGAAAATGTTAGAGGAGC
ACAAGGAGCCGTCACCTCTCATTACCGCCGAGGACGTACAAGAAG
CTAAGTGCGCAGCCGATGAGGCTAAGGAGGTGCGTGAAGCCGAGG
AGTTGCGCGCAGCTCTACCACCTTTGGCAGCTGATGTTGAGGAGC
CCACTCTGGAgGCaGAcGTCGACTTGATGTTACAAGAGGCTGGGG
CCGGCTCAGTGGAGACACCTCGTGGCTTGATAAAGGTTACCAGCT
ACGATGGCGAGGACAAGATCGGCTCTTACGCTGTGCTTTCTCCGC
AGGCTGTACTCAAGAGTGAAAAATTATCTTGCATCCACCCTCTCG
CTGAACAAGTCATAGTGATAACACACTCTGGCCGAAAAGGGCGTT
ATGCCGTGGAACCATACCATGGTAAAGTAGTGGTGCCAGAGGGAC
ATGCAATACCCGTCCAGGACTTTCAAGCTCTGAGTGAAAGTGCCA
CCATTGTGTACAACGAACGTGAGTTCGTAAACAGGTACCTGCACC
ATATTGCCACACATGGAGGAGCGCTGAACACTGATGAAGAATATT
ACAAAACTGTCAAGCCCAGCGAGCACGACGGCGAATACCTGTACG
ACATCGACAGGAAACAGTGCGTCAAGAAAGAACTAGTCACTGGGC
TAGGGCTCACAGGCGAGCTGGTGGATCCTCCCTTCCATGAATTCG
CCTACGAGAGTCTGAGAACACGACCAGCCGCTCCTTACCAAGTAC
CAACCATAGGGGTGTATGGCGTGCCAGGATCAGGCAAGTCTGGCA
TCATTAAAAGCGCAGTCACCAAAAAAGATCTAGTGGTGAGCGCCA
AGAAAGAAAACTGTGCAGAAATTATAAGGGACGTCAAGAAAATGA
AAGGGCTGGACGTCAATGCCAGAACTGTGGACTCAGTGCTCTTGA
ATGGATGCAAACACCCCGTAGAGACCCTGTATATTGACGAAGCTT
TTGCTTGTCATGCAGGTACTCTCAGAGCGCTCATAGCCATTATAA
GACCTAAAAAGGCAGTGCTCTGCGGGGATCCCAAACAGTGCGGTT
TTTTTAACATGATGTGCCTGAAAGTGCATTTTAACCACGAGATTT
GCACACAAGTCTTCCACAAAAGCATCTCTCGCCGTTGCACTAAAT
CTGTGACTTCGGTCGTCTCAACCTTGTTTTACGACAAAAAAATGA
GAACGACGAATCCGAAAGAGACTAAGATTGTGATTGACACTACCG
GCAGTACCAAACCTAAGCAGGACGATCTCATTCTCACTTGTTTCA
GAGGGTGGGTGAAGCAGTTGCAAATAGATTACAAAGGCAACGAAA
TAATGACGGCAGCTGCCTCTCAAGGGCTGACCCGTAAAGGTGTGT
ATGCCGTTCGGTACAAGGTGAATGAAAATCCTCTGTACGCACCCA
CCTCAGAACATGTGAACGTCCTACTGACCCGCACGGAGGACCGCA
TCGTGTGGAAAACACTAGCCGGCGACCCATGGATAAAAACACTGA
CTGCCAAGTACCCTGGGAATTTCACTGCCACGATAGAGGAGTGGC
AAGCAGAGCATGATGCCATCATGAGGCACATCTTGGAGAGACCGG
ACCCTACCGACGTCTTCCAGAATAAGGCAAACGTGTGTTGGGCCA
AGGCTTTAGTGCCGGTGCTGAAGACCGCTGGCATAGACATGACCA
CTGAACAATGGAACACTGTGGATTATTTTGAAACGGACAAAGCTC
ACTCAGCAGAGATAGTATTGAACCAACTATGCGTGAGGTTCTTTG
GACTCGATCTGGACTCCGGTCTATTTTCTGCACCCACTGTTCCGT
TATCCATTAGGAATAATCACTGGGATAACTCCCCGTCGCCTAACA
TGTACGGGCTGAATAAAGAAGTGGTCCGTCAGCTCTCTCGCAGGT
ACCCACAACTGCCTCGGGCAGTTGCCACTGGAAGAGTCTATGACA
TGAACACTGGTACACTGCGCAATTATGATCCGCGCATAAACCTAG
TACCTGTAAACAGAAGACTGCCTCATGCTTTAGTCCTCCACCATA
ATGAACACCCACAGAGTGACTTTTCTTCATTCGTCAGCAAATTGA
AGGGCAGAACTGTCCTGGTGGTCGGGGAAAAGTTGTCCGTCCCAG
GCAAAATGGTTGACTGGTTGTCAGACCGGCCTGAGGCTACCTTCA
GAGCTCGGCTGGATTTAGGCATCCCAGGTGATGTGCCCAAATATG
ACATAATATTTGTTAATGTGAGGACCCCATATAAATACCATCACT
ATCAGCAGTGTGAAGACCATGCCATTAAGCTTAGCATGTTGACCA
AGAAAGCTTGTCTGCATCTGAATCCCGGCGGAACCTGTGTCAGCA
TAGGTTATGGTTACGCTGACAGGGCCAGCGAAAGCATCATTGGTG
CTATAGCGCGGCAGTTCAAGTTTTCCCGGGTATGCAAACCGAAAT
CCTCACTTGAAGAGACGGAAGTTCTGTTTGTATTCATTGGGTACG
ATCGCAAGGCCCGTACGCACAATTCTTACAAGCTTTCATCAACCT
TGACCAACATTTATACAGGTTCCAGACTCCACGAAGCCGGATGTG
CACCCTCATATCATGTGGTGCGAGGGGATATTGCCACGGCCACCG
AAGGAGTGATTATAAATGCTGCTAACAGCAAAGGACAACCTGGCG
GAGGGGTGTGCGGAGCGCTGTATAAGAAATTCCCGGAAAGCTTCG
ATTTACAGCCGATCGAAGTAGGAAAAGCGCGACTGGTCAAAGGTG
CAGCTAAACATATCATTCATGCCGTAGGACCAAACTTCAACAAAG
TTTCGGAGGTTGAAGGTGACAAACAGTTGGCAGAGGCTTATGAGT
CCATCGCTAAGATTGTCAACGATAACAATTACAAGTCAGTAGCGA
TTCCACTGTTGTCCACCGGCATCTTTTCCGGGAACAAAGATCGAC
TAACCCAATCATTGAACCATTTGCTGACAGCTTTAGACACCACTG
ATGCAGATGTAGCCATATACTGCAGGGACAAGAAATGGGAAATGA
CTCTCAAGGAAGCAGTGGCTAGGAGAGAAGCAGTGGAGGAGATAT
GCATATCCGACGACTCTTCAGTGACAGAACCTGATGCAGAGCTGG
TGAGGGTGCATCCGAAGAGTTCTTTGGCTGGAAGGAAGGGCTACA
GCACAAGCGATGGCAAAACTTTCTCATATTTGGAAGGGACCAAGT
TTCACCAGGCGGCCAAGGATATAGCAGAAATTAATGCCATGTGGC
CCGTTGCAACGGAGGCCAATGAGCAGGTATGCATGTATATCCTCG
GAGAAAGCATGAGCAGTATTAGGTCGAAATGCCCCGTCGAAGAGT
CGGAAGCCTCCACACCACCTAGCACGCTGCCTTGCTTGTGCATCC
ATGCCATGACTCCAGAAAGAGTACAGCGCCTAAAAGCCTCACGTC
CAGAACAAATTACTGTGTGCTCATCCTTTCCATTGCCGAAGTATA
GAATCACTGGTGTGCAGAAGATCCAATGCTCCCAGCCTATATTGT
TCTCACCGAAAGTGCCTGCGTATATTCATCCAAGGAAGTATCTCG
TGGAAACACCACCGGTAGACGAGACTCCGGAGCCATCGGCAGAGA
ACCAATCCACAGAGGGGACACCTGAACAACCACCACTTATAACCG
AGGATGAGACCAGGACTAGAACGCCTGAGCCGATCATCATCGAAG
AGGAAGAAGAGGATAGCATAAGTTTGCTGTCAGATGGCCCGACCC
ACCAGGTGCTGCAAGTCGAGGCAGACATTCACGGGCCGCCCTCTG
TATCTAGCTCATCCTGGTCCATTCCTCATGCATCCGACTTTGATG
TGGACAGTTTATCCATACTTGACACCCTGGAGGGAGCTAGCGTGA
CCAGCGGGGCAACGTCAGCCGAGACTAACTCTTACTTCGCAAAGA
GTATGGAGTTTCTGGCGCGACCGGTGCCTGCGCCTCGAACAGTAT
TCAGGAACCCTCCACATCCCGCTCCGCGCACAAGAACACCGTCAC
TTGCACCCAGCAGGGCCTGCTCGAGAACCAGCCTAGTTTCCACCC
CGCCAGGCGTGAATAGGGTGATCACTAGAGAGGAGCTCGAGGCGC
TTACCCCGTCACGCACTCCTAGCAGGTCGGTCTCGAGAACCAGCC
TGGTCTCCAACCCGCCAGGCGTAAATAGGGTGATTACAAGAGAGG
AGTTTGAGGCGTTCGTAGCACAACAACAATGACGGTTTGATGCGG
GTGCATACATCTTTTCCTCCGACACCGGTCAAGGGCATTTACAAC
AAAAATCAGTAAGGCAAACGGTGCTATCCGAAGTGGTGTTGGAGA
GGACCGAATTGGAGATTTCGTATGCCCCGCGCCTCGACCAAGAAA
AAGAAGAATTACTACGCAAGAAATTACAGTTAAATCCCACACCTG
CTAACAGAAGCAGATACCAGTCCAGGAAGGTGGAGAACATGAAAG
CCATAACAGCTAGACGTATTCTGCAAGGCCTAGGGCATTATTTGA
AGGCAGAAGGAAAAGTGGAGTGCTACCGAACCCTGCATCCTGTTC
CTTTGTATTCATCTAGTGTGAACCGTGCCTTTTCAAGCCCCAAGG
TCGCAGTGGAAGCCTGTAACGCCATGTTGAAAGAGAACTTTCCGA
CTGTGGCTTCTTACTGTATTATTCCAGAGTACGATGCCTATTTGG
ACATGGTTGACGGAGCTTCATGCTGCTTAGACACTGCCAGTTTTT
GCCCTGCAAAGCTGCGCAGCTTTCCAAAGAAACACTCCTATTTGG
AACCCACAATACGATCGGCAGTGCCTTCAGCGATCCAGAACACGC
TCCAGAACGTCCTGGCAGCTGCCACAAAAAGAAATTGCAATGTCA
CGCAAATGAGAGAATTGCCCGTATTGGATTCGGCGGCCTTTAATG
TGGAATGCTTCAAGAAATATGCGTGTAATAATGAATATTGGGAAA
CGTTTAAAGAAAACCCCATCAGGCTTACTGAAGAAAACGTGGTAA
ATTACATTACCAAATTAAAAGGACCAAAAGCTGCTGCTCTTTTTG
CGAAGACACATAATTTGAATATGTTGCAGGACATACCAATGGACA
GGTTTGTAATGGACTTAAAGAGAGACGTGAAAGTGACTCCAGGAA
CAAAACATACTGAAGAACGGCCCAAGGTACAGGTGATCCAGGCTG
CCGATCCGCTAGCAACAGCGTATCTGTGCGGAATCCACCGAGAGC
TGGTTAGGAGATTAAATGCGGTCCTGCTTCCGAACATTCATACAC
TGTTTGATATGTCGGCTGAAGACTTTGACGCTATTATAGCCGAGC
ACTTCCAGCCTGGGGATTGTGTTCTGGAAACTGACATCGCGTCGT
TTGATAAAAGTGAGGACGACGCCATGGCTCTGACCGCGTTAATGA
TTCTGGAAGACTTAGGTGTGGACGCAGAGCTGTTGACGCTGATTG
AGGCGGCTTTCGGCGAAATTTCATCAATACATTTGCCCACTAAAA
CTAAATTTAAATTCGGAGCCATGATGAAATCTGGAATGTTCCTCA
CACTGTTTGTGAACACAGTCATTAACATTGTAATCGCAAGCAGAG
TGTTGAGAGAACGGCTAACCGGATCACCATGTGCAGCATTCATTG
GAGATGACAATATCGTGAAAGGAGTCAAATCGGACAAATTAATGG
CAGACAGGTGCGCCACCTGGTTGAATATGGAAGTCAAGATTATAG
ATGCTGTGGTGGGCGAGAAAGCGCCTTATTTCTGTGGAGGGTTTA
TTTTGTGTGACTCCGTGACCGGCACAGCGTGCCGTGTGGCAGACC
CCCTAAAAAGGCTGTTTAAGCTTGGCAAACCTCTGGCAGCAGACG
ATGAACATGATGATGACAGGAGAAGGGCATTGCATGAAGAGTCAA
CACGCTGGAACCGAGTGGGTATTCTTTCAGAGCTGTGCAAGGCAG
TAGAATCAAGGTATGAAACCGTAGGAACTTCCATCATAGTTATGG
CCATGACTACTCTAGCTAGCAGTGTTAAATCATTCAGCTACCTGA
GAGGGGCCCCTATAACTCTCTACGGCTAACCTGAATGGACTACGA
CATAGTCTAGTCCGCCAAGTCTAGCAT
[SEQ ID No: 266]
---GOI----
CGGAGACGGCGCAGAAGAAGAGGATCTGGCGAAGGCAGAGGCAGCC
TGCTtACATGtGGcGACGTGGAAGAGAACCCCGGACCTATGGGCG
ATAGCAGCCCCGATACCTTTTCCGATGGCCTGAGCAGCAGCACCC
TGCCTGATGATCACAGCAGCTACACCGTGCCTGGCTACATGCAGG
ACCTGGAAGTGGAACAGGCCCTGACACCAGCTCTGAGCCCTTGTG
CTGTGTCCAGCACACTGCCCGATTGGCACATCCCTGTGGAAGTGG
TGCCTGACAGCACCAGCGACCTGTACAACTTCCAAGTGTCCCCTA
TGCCTAGCACCTCCGAGGCCACCACCGATGAGGATGAAGAGGGAA
AGCTGCCCGAGGACATCATGAAGCTGCTGGAACAGAGCGAGTGGC
AGCCCACCAATGTGGATGGCAAGGGCTACCTGCTGAACGAGCCTG
GCGTTCAGCCTACAAGCGTGTACGGCGACTTCAGCTGCAAAGAGG
AACCCGAGATCGATAGCCCTGGCGGCGATATCGGACTGAGCCTGC
AGAGAGTGTTCACCGACCTGAAGAACATGGACGCCACCTGGCTGG
ACAGCCTGCTGACACCTGTTAGACTGCCCTCTATCCAGGCTATCC
CCTGCGCTCCTTGAGCGGCCGCGAATTGGCAAGCTGCTTACATAG
AACTCGCGGCGATTGGCATGCCGCCTTAAAATTTTTATTTTATTT
TTCTTTTCTTTTCCGAATCGGATTTTGTTTTTAATATTTCAAAAA
AAAAAAAAAAAAAAAAAAAAAAAAAAAAAACGCGTCGAGGGGAAT
TAATTCTTGAAGACGAAAGGGCCAGGTGGCACTTTTCGGGGAAAT
GTGCGCGGAACCCCTATTTGTTTATTTTTCTAAATACATTCAAAT
ATGTATCCGCTCATGAGACAATAACCCTGATAAATGCTTCAATAA
TATTGAAAAAGGAAGAGTATGAGTATTCAACATTTCCGTGTCGCC
CTTATTCCCTTTTTTGCGGCATTTTGCCTTCCTGTTTTTGCTCAC
CCAGAAACGCTGGTGAAAGTAAAAGATGCTGAAGATCAGTTGGGT
GCACGAGTGGGTTACATCGAACTGGATCTCAACAGCGGTAAGATC
CTTGAGAGTTTTCGCCCCGAAGAACGTTTTCCAATGATGAGCACT
TTTAAAGTTCTGCTATGTGGCGCGGTATTATCCCGTGTTGACGCC
GGGCAAGAGCAACTCGGTCGCCGCATACACTATTCTCAGAATGAC
TTGGTTGAGTACTCACCAGTCACAGAAAAGCATCTTACGGATGGC
ATGACAGTAAGAGAATTATGCAGTGCTGCCATAACCATGAGTGAT
AACACTGCGGCCAACTTACTTCTGACAACGATCGGAGGACCGAAG
GAGCTAACCGCTTTTTTGCACAACATGGGGGATCATGTAACTCGC
CTTGATCGTTGGGAACCGGAGCTGAATGAAGCCATACCAAACGAC
GAGCGTGACACCACGATGCCTGTAGCAATGGCAACAACGTTGCGC
AAACTATTAACTGGCGAACTACTTACTCTAGCTTCCCGGCAACAA
TTAATAGACTGGATGGAGGCGGATAAAGTTGCAGGACCACTTCTG
CGCTCGGCCCTTCCGGCTGGCTGGTTTATTGCTGATAAATCTGGA
GCCGGTGAGCGTGGGTCTCGCGGTATCATTGCAGCACTGGGGCCA
GATGGTAAGCCCTCCCGTATCGTAGTTATCTACACGACGGGGAGT
CAGGCAACTATGGATGAACGAAATAGACAGATCGCTGAGATAGGT
GCCTCACTGATTAAGCATTGGTAACTGTCAGACCAAGTTTACTCA
TATATACTTTAGATTGATTTAAAACTTCATTTTTAATTTAAAAGG
ATCTAGGTGAAGATCCTTTTTGATAATCTCATGACCAAAATCCCT
TAACGTGAGTTTTCGTTCCACTGAGCGTCAGACCCCGTAGAAAAG
ATCAAAGGATCTTCTTGAGATCCTTTTTTTCTGCGCGTAATCTGC
TGCTTGCAAACAAAAAAACCACCGCTACCAGCGGTGGTTTGTTTG
CCGGATCAAGAGCTACCAACTCTTTTTCCGAAGGTAACTGGCTTC
AGCAGAGCGCAGATACCAAATACTGTCCTTCTAGTGTAGCCGTAG
TTAGGCCACCACTTCAAGAACTCTGTAGCACCGCCTACATACCTC
GCTCTGCTAATCCTGTTACCAGTGGCTGCTGCCAGTGGCGATAAG
TCGTGTCTTACCGGGTTGGACTCAAGACGATAGTTACCGGATAAG
GCGCAGCGGTCGGGCTGAACGGGGGGTTCGTGCACACAGCCCAGC
TTGGAGCGAACGACCTACACCGAACTGAGATACCTACAGCGTGAG
CATTGAGAAAGCGCCACGCTTCCCGAAGGGAGAAAGGCGGACAGG
TATCCGGTAAGCGGCAGGGTCGGAACAGGAGAGCGCACGAGGGAG
CTTCCAGGGGGAAACGCCTGGTATCTTTATAGTCCTGTCGGGTTT
CGCCACCTCTGACTTGAGCGTCGATTTTTGTGATGCTCGTCAGGG
GGGCGGAGCCTATGGAAAAACGCCATTCTAGAATGGCGCGCCCTT
AAGGGGAGAATAGGAGCCGCAACACACAAGCAACGCGAGGTCGTT
TAAAC

Accordingly, preferably the vector comprises the nucleotide sequence substantially as set out above, comprising or consisting of SEQ ID NO: 231, a GOI, and SEQ ID No: 266, or a variant or fragment thereof.

The saRNA constructs of the invention may be made using a DNA plasmid, as a template. RNA copies may then be made by in vitro transcription using a polymerase, such as T7 polymerase, and the T7 promoter may be upstream of the saRNA. Hence, the saRNA constructs of the invention may be made using a DNA plasmid having a nucleic acid sequence as set out in any one of SEQ ID No: 1 to 266, such as the sequence substantially as set out above, comprising or consisting of SEQ ID No: 231, a GOI, and SEQ ID No: 266, or a variant or fragment thereof, as the template. Of course, it will be appreciated that other RNA polymerases could be used instead of T7 polymerase, for example the SP6 or the T3 polymerase, in which case the saRNA construct may comprise the SP6 or T3 promoter instead.

The vector of the fourth aspect encoding the RNA construct of the first aspect may for example be a plasmid, cosmid or phage and/or be a viral vector. Such recombinant vectors are highly useful in the delivery systems of the invention for transforming cells with the nucleotide sequences. The nucleotide sequences may preferably be a DNA sequence, and it is this DNA sequence which encodes the RNA sequence forming the RNA construct of the first aspect.

Recombinant vectors encoding the RNA construct of the first aspect may also include other functional elements. For example, they may further comprise a variety of other functional elements including a suitable promoter for initiating transgene expression upon introduction of the vector in a host cell. For instance, the vector is preferably capable of autonomously replicating in the nucleus of the host cell, such as a bacterial cell. In this case, elements which induce or regulate DNA replication may be required in the recombinant vector. Alternatively, the recombinant vector may be designed such that it integrates into the genome of a host cell. In this case, DNA sequences which favour targeted integration (e.g. by homologous recombination) are envisaged. Suitable promoters may include the SV40 promoter, CMV, EF1a, PGK, viral long terminal repeats, as well as inducible promoters, such as the Tetracycline inducible system, as examples.

The cassette or vector may also comprise a terminator, such as the Beta globin, SV40 polyadenylation sequences or synthetic polyadenylation sequences. The recombinant vector may also comprise a promoter or regulator or enhancer to control expression of the nucleic acid as required.

The vector may also comprise DNA coding for a gene that may be used as a selectable marker in the cloning process, i.e. to enable selection of cells that have been transfected or transformed, and to enable the selection of cells harbouring vectors incorporating heterologous DNA. For example, ampicillin, neomycin, puromycin or chloramphenicol resistance is envisaged. Alternatively, the selectable marker gene may be in a different vector to be used simultaneously with the vector containing the transgene(s). The cassette or vector may also comprise DNA involved with regulating expression of the nucleotide sequence, or for targeting the expressed polypeptide to a certain part of the host cell.

Purified vector may be inserted directly into a host cell by suitable means, e.g. direct endocytotic uptake. The vector may be introduced directly into a host cell (e.g. a eukaryotic or prokaryotic cell) by transfection, infection, electroporation, microinjection, cell fusion, protoplast fusion or ballistic bombardment. Alternatively, vectors of the invention may be introduced directly into a host cell using a particle gun.

The nucleic acid molecule may (but not necessarily) be one, which becomes incorporated in the DNA of the host cell. Undifferentiated cells may be stably transformed leading to the production of genetically modified daughter cells (in which case regulation of expression in the subject may be required e.g. with specific transcription factors or gene activators). Alternatively, the delivery system may be designed to favour unstable or transient transformation of differentiated cells. When this is the case, regulation of expression may be less important because expression of the DNA molecule will stop when the transformed cells die or stop expressing the protein.

Alternatively, the delivery system may provide the nucleic acid molecule to the host cell without it being incorporated in a vector. For instance, the nucleic acid molecule may be incorporated within a liposome or virus particle. Alternatively, a “naked” nucleic acid molecule may be inserted into a host cell by a suitable means e.g. direct endocytotic uptake.

In a fifth aspect, there is provided a pharmaceutical composition comprising the RNA construct of the first aspect, the nucleic acid sequence of the second aspect, the expression cassette of the third aspect or the vector of the fourth aspect, and a pharmaceutically acceptable vehicle.

In a sixth aspect, there is provided a process for making the pharmaceutical composition according to the fifth aspect, the method comprising contacting the RNA construct of the first aspect, the nucleic acid sequence of the second aspect, the expression cassette of the third aspect or the vector of the fourth aspect, with a pharmaceutically acceptable vehicle.

In a seventh aspect, there is provided a method of preparing the RNA construct of the first aspect, the method comprising:
a) i) introducing, into a host cell, the vector of the fourth aspect; and
   ii) culturing the host cell under conditions to result in the production of the RNA construct of the first aspect; or
b) transcribing the RNA construct from the vector according to the fourth aspect.

The host cell of step a) may be a eukaryotic or prokaryotic host cell. Preferably, the host cell is a eukaryotic host cell. More preferably, the host cell is a mammalian host cell such as Human embryonic kidney 293 cells or Chinese hamster ovary (CHO) cells. Step (b) may be performed in vitro or in vivo, preferably in vitro.

Suitable methods of in vitro transcription are well known in the art and would be known to those skilled in the art. For example, as described in Molecular Cloning, A Laboratory Manual, 2nd edition. (1989) editor C Nolan, Cold Spring Harbor Laboratory Press.

The RNA replicon of the first aspect is particularly suitable for therapy.

While the inventors envisaged that the RNA construct of the first aspect would be generated by in vitro transcription for in vivo use in therapy, those experienced in the art will recognise that the RNA construct can be generated in vivo in a subject for therapy, by in vivo delivery of the nucleic acid according to the second aspect, the expression cassette according to the third aspect, or the vector according to the fourth aspect to a subject.

Hence, according to an eighth aspect, there is provided a RNA construct according to the first aspect, the nucleic acid according to the second aspect, the expression cassette according to the third aspect, the vector according to the fourth aspect or the pharmaceutical composition according to the fifth aspect, for use as a medicament or in therapy.

In a ninth aspect of the invention, there is provided a RNA construct according to the first aspect, the nucleic acid according to the second aspect, the expression cassette according to the third aspect, the vector according to the fourth aspect or the pharmaceutical composition according to the fifth aspect, for use in the prevention, amelioration or treatment of a protozoan, fungal, bacterial or viral infection.

The protozoan, fungal, bacterial or viral infection may be an infection of a protozoa, fungus, bacterium or virus as defined in the first aspect.

In a tenth aspect of the invention, there is provided an RNA construct according to the first aspect, the nucleic acid according to the second aspect, the expression cassette according to the third aspect, the vector according to the fourth aspect or the pharmaceutical composition according to the fifth aspect, for use in the prevention, amelioration or treatment of cancer.

The cancer may be as defined in the first aspect.

In an eleventh aspect of the invention, there is provided a method for treating a protozoan, fungal, bacterial or viral infection, the method comprising administering, to a subject in need thereof, a therapeutically effective amount of the RNA construct according to the first aspect, the nucleic acid according to the second aspect, the expression cassette according to the third aspect, the vector according to the fourth aspect or the pharmaceutical composition according to the fifth aspect.

The protozoan, fungal, bacterial or viral infection to be treated may be an infection of a protozoa, fungus, bacterium or virus as defined in the first aspect.

In a twelfth aspect of the invention, there is provided a method for treating cancer, the method comprising administering, to a subject in need thereof, a therapeutically effective amount of the RNA construct according to the first aspect, the nucleic acid according to the second aspect, the expression cassette according to the third aspect, the vector according to the fourth aspect or the pharmaceutical composition according to the fifth aspect.

The cancer to be treated may be as defined in the first aspect.

The RNA construct described herein provides an effective means of vaccinating a subject (e.g. against a viral, bacterial or fungal infection) and cancer.

Accordingly, in a thirteenth aspect of the invention, there is provided a vaccine comprising the RNA construct according to the first aspect, the nucleic acid according to the second aspect, the expression cassette according to the third aspect, the vector according to the fourth aspect or the pharmaceutical composition according to the fifth aspect.

The adjuvant incorporated into a delivery formulation may be selected form the group consisting of a bacterial lipopeptide, lipoprotein and lipoteichoic acid; mycobacterial lipoglycan; yeast zymosan, porin, Lipopolysaccharide, Lipid A, monophosphoryl lipid A (MPL), Flagellin, CpG DNA, hemozoin, Tomatine, ISCOM, ISCOMATRIX™, squalene based emulsions, polymers such as PEI, Carbopol, lipid nanoparticles and bacterial toxins (CT, LT). Further examples of adjuvants incorporated into the delivery formulation may include an aluminium salt, a synthetic form of DNA, a carbohydrate, a tablet binder, an ion exchange resin, a preservative, a polymer, an emulsion and/or a lipid. Examples of adjuvants may include monosodium glutamate, sucrose, dextrose, aluminum bovine, human serum albumin, cytosine phosphoguanine, potassium phosphate, plasdone C, anhydrous lactose, cellulose, polacrilin potassium, glycerine, asparagine, citric acid, potassium phosphate magnesium sulfate, iron ammonium citrate, 2-phenoxyethanol, aluminium, beta-propiolactone, bovine extract, DOPC, EDTA, formaldehyde, thimerosal, phenol, potassium aluminum sulfate, potassium glutamate, sodium borate, sodium metabisulphite, urea, PLGA, PVA, PLA, PVP, cyclodextrin-based stabilisers, oil in water emulsion adjuvants and/or lipid-based adjuvants.

In a fourteenth aspect of the invention, there is provided an RNA construct according to the first aspect, the nucleic acid according to the second aspect, the expression cassette according to the third aspect, the vector according to the fourth aspect or the pharmaceutical composition according to the fifth aspect, for use in stimulating an immune response in a subject.

The immune response may be stimulated against a protozoa, bacterium, virus, fungus or cancer as per the antigens defined in the first aspect.

According to a fifteenth aspect, there is provided an RNA construct according to the first aspect, the nucleic acid according to the second aspect, the expression cassette according to the third aspect, the vector according to the fourth aspect or the pharmaceutical composition according to the fifth aspect, for use in stem cell therapy.

Stem cell therapy may relate to the reprogramming somatic cells to cells having stem cell characteristics.

Somatic cells may be reprogrammed by delivering one or more proteins that are capable of enhancing reprogramming of somatic cells to cells having stem cell characteristics as defined in the first aspect.

According to a sixteenth aspect, there is provided a method of modifying a cell ex vivo or in vitro, comprising delivering, to the cell, the RNA construct according to the first aspect, the nucleic acid according to the second aspect, the expression cassette according to the third aspect, the vector according to the fourth aspect or the pharmaceutical composition according to the fifth aspect.

Preferably, the method is performed ex vivo.

The cell may be a eukaryotic or prokaryotic cell. Preferably, the cell is a eukaryotic cell. More preferably, the cell is a mammalian host cell. Most preferably, the cell is a human cell.

Preferably, the modified cell is suitable for cell-therapy indications.

In a seventeenth aspect, there is provided a modified cell obtained from, or obtainable by, the method of the sixteenth aspect.

In an eighteenth aspect, there is provided the modified cell of the seventeenth aspect, for use in therapy, optionally cell therapy.

It will be appreciated that the RNA construct according to the first aspect, the nucleic acid according to the second aspect, the expression cassette according to the third aspect, the vector according to the fourth aspect or the pharmaceutical composition according to the fifth aspect (herein known as the active agents) may be used in a medicament, which may be used as a monotherapy (i.e. use of the active agent), for treating, ameliorating, or preventing disease or vaccination. Alternatively, the active agents according to the invention may be used as an adjunct to, or in combination with, known therapies for treating, ameliorating, or preventing disease.

The RNA construct, nucleic acid sequence, expression cassette, vector or pharmaceutical composition of the invention may be combined in compositions having a number of different forms depending, in particular, on the manner in which the composition is to be used. Thus, for example, the composition may be in the form of a powder, tablet, capsule, liquid, ointment, cream, gel, hydrogel, aerosol, spray, micellar solution, transdermal patch, liposome suspension, polyplex, emulsion, lipid nanoparticles (with RNA on the surface or encapsulated) or any other suitable form that may be administered to a person or animal in need of treatment or vaccination. It will be appreciated that the vehicle of medicaments according to the invention should be one which is well-tolerated by the subject to whom it is given.

The RNA construct, nucleic acid sequence, expression cassette, vector or pharmaceutical composition of the invention may also be incorporated within a slow- or delayed-release device. Such devices may, for example, be inserted on or under the skin, and the medicament may be released over weeks or even months. The device may be located at least adjacent the treatment site. Such devices may be particularly advantageous when long-term treatment with the genetic construct or the recombinant vector is required and which would normally require frequent administration (e.g. at least daily injection).

In a preferred embodiment, however, medicaments according to the invention may be administered to a subject by injection into the blood stream, muscle, skin or directly into a site requiring treatment. Most preferably, the medicaments, including the RNA construct, are injected into muscle. Injections may be intravenous (bolus or infusion) or subcutaneous (bolus or infusion), or intradermal (bolus or infusion), or intramuscular (bolus or infusion).

It will be appreciated that the amount of RNA construct, nucleic acid sequence, expression cassette, vector or pharmaceutical composition that is required is determined by its biological activity and bioavailability, which in turn depends on the mode of administration, the physiochemical properties of the RNA construct, nucleic acid sequence, expression cassette, vector or pharmaceutical composition and whether it is being used as a monotherapy or in a combined therapy. The frequency of administration will also be influenced by the half-life of the active agent within the subject being treated. Optimal dosages to be administered may be determined by those skilled in the art, and will vary with the particular the RNA construct, nucleic acid sequence, expression cassette, vector or pharmaceutical composition in use, the strength of the pharmaceutical composition, the mode of administration, and the type and advancement of the viral infection. Additional factors depending on the particular subject being treated will result in a need to adjust dosages, including subject age, weight, gender, diet, and time of administration.

Generally, a daily dose of between 0.00 μg/kg of body weight and 10 mg/kg of body weight, or between 0.01 μg/kg of body weight and 1 mg/kg of body weight, of the RNA construct, nucleic acid sequence, expression cassette, vector or pharmaceutical composition of the invention may be used for treating, ameliorating, or preventing a disease, depending upon the active agent used.

Daily doses may be given as a single administration (e.g. a single daily injection or inhalation of a nasal spray). Alternatively, the RNA construct, nucleic acid sequence, expression cassette, vector or pharmaceutical composition may require administration twice or more times during a day. As an example, the RNA construct, nucleic acid sequence, expression cassette, vector or pharmaceutical composition may be administered as two (or more depending upon the severity of the disease being treated) daily doses of between 0.07 μg and 700 mg (i.e. assuming a body weight of 70 kg). A patient receiving treatment may take a first dose upon waking and then a second dose in the evening (if on a two dose regime) or at 3- or 4-hourly intervals thereafter. Alternatively, a slow release device may be used to provide optimal doses of the RNA construct, nucleic acid sequence, expression cassette, vector or pharmaceutical composition according to the invention to a patient without the need to administer repeated doses.

Preferably, however, the RNA construct, nucleic acid sequence, expression cassette, vector or pharmaceutical composition according to the invention may be given as a weekly dose, and more preferably a fortnightly dose.

Known procedures, such as those conventionally employed by the pharmaceutical industry (e.g. in vivo experimentation, clinical trials, etc.), may be used to form specific formulations of the RNA construct, nucleic acid sequence, expression cassette or vector according to the invention and precise therapeutic regimes (such as daily doses of the agents and the frequency of administration).

A “subject” may be a vertebrate, mammal, or domestic animal. Hence, compositions and medicaments according to the invention may be used to treat any mammal, for example livestock (e.g. a horse), pets, or may be used in other veterinary applications. Most preferably, however, the subject is a human being.

A “therapeutically effective amount” of the RNA construct, nucleic acid sequence, expression cassette, vector or pharmaceutical composition is any amount which, when administered to a subject, is the amount of the aforementioned that is needed to ameliorate, prevent or treat any given disease.

For example, the RNA construct, nucleic acid sequence, expression cassette, vector or pharmaceutical composition of the invention may be used may be from about 0.0001 mg to about 800 mg, and preferably from about 0.001 mg to about 500 mg. It is preferred that the amount of the replicon, nucleic acid sequence, expression cassette, vector or pharmaceutical composition is an amount from about 0.01 mg to about 250 mg, and most preferably from about 0.01 mg to about 1 mg. Preferably, the RNA construct, nucleic acid sequence, expression cassette, vector or pharmaceutical composition according to the invention is administered at a dose of 1-200 g.

A “pharmaceutically acceptable vehicle” as referred to herein, is any known compound or combination of known compounds that are known to those skilled in the art to be useful in formulating pharmaceutical compositions.

In one embodiment, the pharmaceutically acceptable vehicle may be a solid, and the composition may be in the form of a powder or tablet. A solid pharmaceutically acceptable vehicle may include one or more substances which may also act as flavouring agents, lubricants, solubilisers, suspending agents, dyes, fillers, glidants, compression aids, inert binders, sweeteners, preservatives, dyes, coatings, or tablet-disintegrating agents. The vehicle may also be an encapsulating material. In powders, the vehicle is a finely divided solid that is in admixture with the finely divided active agents according to the invention. In tablets, the active agent (e.g. RNA construct, nucleic acid sequence, expression cassette, vector or pharmaceutical composition according to the invention) may be mixed with a vehicle having the necessary compression properties in suitable proportions and compacted in the shape and size desired. The powders and tablets preferably contain up to 99% of the active agents. Suitable solid vehicles include, for example calcium phosphate, magnesium stearate, talc, sugars, lactose, dextrin, starch, gelatin, cellulose, polyvinylpyrrolidine, low melting waxes and ion exchange resins. In another embodiment, the pharmaceutical vehicle may be a gel and the composition may be in the form of a cream or the like.

However, the pharmaceutical vehicle may be a liquid, and the pharmaceutical composition is in the form of a solution. Liquid vehicles are used in preparing solutions, suspensions, emulsions, syrups, elixirs and pressurized compositions. The RNA construct, nucleic acid sequence, expression cassette, vector or pharmaceutical composition according to the invention may be dissolved or suspended in a pharmaceutically acceptable liquid vehicle such as water, an organic solvent, a mixture of both or pharmaceutically acceptable oils or fats. The liquid vehicle can contain other suitable pharmaceutical additives such as solubilisers, emulsifiers, buffers, preservatives, sweeteners, flavouring agents, suspending agents, thickening agents, colours, viscosity regulators, stabilizers or osmo-regulators. Suitable examples of liquid vehicles for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the vehicle can also be an oily ester such as ethyl oleate and isopropyl myristate. Sterile liquid vehicles are useful in sterile liquid form compositions for parenteral administration. The liquid vehicle for pressurized compositions can be a halogenated hydrocarbon or other pharmaceutically acceptable propellant.

Liquid pharmaceutical compositions, which are sterile solutions or suspensions, can be utilized by, for example, subcutaneous, intradermal, intrathecal, epidural, intraperitoneal, intravenous and particularly intramuscular injection. The nucleic acid sequence, or expression cassette of the invention may be prepared as a sterile solid composition that may be dissolved or suspended at the time of administration using sterile water, saline, or other appropriate sterile injectable medium.

The RNA construct, nucleic acid sequence, expression cassette, vector or pharmaceutical composition of the invention may be administered orally in the form of a sterile solution or suspension containing other solutes or suspending agents (for example, enough saline or glucose to make the solution isotonic), bile salts, acacia, gelatin, sorbitan monoleate, polysorbate 80 (oleate esters of sorbitol and its anhydrides copolymerized with ethylene oxide) and the like. The RNA construct, nucleic acid sequence, expression cassette, vector or pharmaceutical composition according to the invention can also be administered orally either in liquid or solid composition form. Compositions suitable for oral administration include solid forms, such as pills, capsules, granules, tablets, and powders, and liquid forms, such as solutions, syrups, elixirs, and suspensions. Forms useful for parenteral administration include sterile solutions, emulsions, and suspensions.

It will be appreciated that the invention extends to any nucleic acid or peptide or variant, derivative or analogue thereof, which comprises substantially the amino acid or nucleic acid sequences of any of the sequences referred to herein, including variants or fragments thereof. The terms “substantially the amino acid/nucleotide/peptide sequence”, “variant” and “fragment”, can be a sequence that has at least 40% sequence identity with the amino acid/nucleotide/peptide sequences of any one of the sequences referred to herein, for example 40% identity with any of the sequences identified herein.

Amino acid/polynucleotide/polypeptide sequences with a sequence identity which is greater than 65%, more preferably greater than 70%, even more preferably greater than 75%, and still more preferably greater than 80% sequence identity to any of the sequences referred to are also envisaged. Preferably, the amino acid/polynucleotide/polypeptide sequence has at least 85% identity with any of the sequences referred to, more preferably at least 90% identity, even more preferably at least 92% identity, even more preferably at least 95% identity, even more preferably at least 97% identity, even more preferably at least 98% identity and, most preferably at least 99% identity with any of the sequences referred to herein.

The skilled technician will appreciate how to calculate the percentage identity between two amino acid/polynucleotide/polypeptide sequences. In order to calculate the percentage identity between two amino acid/polynucleotide/polypeptide sequences, an alignment of the two sequences must first be prepared, followed by calculation of the sequence identity value. The percentage identity for two sequences may take different values depending on:—(i) the method used to align the sequences, for example, ClustalW, BLAST, FASTA, Smith-Waterman (implemented in different programs), or structural alignment from 3D comparison; and (ii) the parameters used by the alignment method, for example, local vs global alignment, the pair-score matrix used (e.g. BLOSUM62, PAM250, Gonnet etc.), and gap-penalty, e.g. functional form and constants.

Having made the alignment, there are many different ways of calculating percentage identity between the two sequences. For example, one may divide the number of identities by: (i) the length of shortest sequence; (ii) the length of alignment; (iii) the mean length of sequence; (iv) the number of non-gap positions; or (v) the number of equivalenced positions excluding overhangs. Furthermore, it will be appreciated that percentage identity is also strongly length dependent. Therefore, the shorter a pair of sequences is, the higher the sequence identity one may expect to occur by chance.

Hence, it will be appreciated that the accurate alignment of protein or DNA sequences is a complex process. The popular multiple alignment program ClustalW (Thompson et al., 1994, Nucleic Acids Research, 22, 4673-4680; Thompson et al., 1997, Nucleic Acids Research, 24, 4876-4882) is a preferred way for generating multiple alignments of proteins or DNA in accordance with the invention. Suitable parameters for ClustalW may be as follows: For DNA alignments: Gap Open Penalty=15.0, Gap Extension Penalty=6.66, and Matrix=Identity. For protein alignments: Gap Open Penalty=10.0, Gap Extension Penalty=0.2, and Matrix=Gonnet. For DNA and Protein alignments: ENDGAP=−1, and GAPDIST=4. Those skilled in the art will be aware that it may be necessary to vary these and other parameters for optimal sequence alignment.

Preferably, calculation of percentage identities between two amino acid/polynucleotide/polypeptide sequences may then be calculated from such an alignment as (N/T)*100, where N is the number of positions at which the sequences share an identical residue, and T is the total number of positions compared including gaps and either including or excluding overhangs. Preferably, overhangs are included in the calculation. Hence, a most preferred method for calculating percentage identity between two sequences comprises (i) preparing a sequence alignment using the ClustalW program using a suitable set of parameters, for example, as set out above; and (ii) inserting the values of N and T into the following formula:—Sequence Identity=(N/T)*100.

Alternative methods for identifying similar sequences will be known to those skilled in the art. For example, a substantially similar nucleotide sequence will be encoded by a sequence which hybridizes to DNA sequences or their complements under stringent conditions. By stringent conditions, the inventors mean the nucleotide hybridises to filter-bound DNA or RNA in 3× sodium chloride/sodium citrate (SSC) at approximately 45° C. followed by at least one wash in 0.2×SSC/0.1% SDS at approximately 20-65° C. Alternatively, a substantially similar polypeptide may differ by at least 1, but less than 5, 10, 20, 50 or 100 amino acids from any of the sequences identified herein.

Due to the degeneracy of the genetic code, it is clear that any nucleic acid sequence described herein could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent (synonymous) change. Other suitable variants are those having homologous nucleotide sequences but comprising all, or portions of, sequence, which are altered by the substitution of different codons that encode an amino acid with a side chain of similar biophysical properties to the amino acid it substitutes, to produce a conservative change. For example, small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid. It will therefore be appreciated which amino acids may be replaced with an amino acid having similar biophysical properties, and the skilled technician will know the nucleotide sequences encoding these amino acids.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying Figures, in which:—

FIG. 1 shows a schematic of various embodiments (denoted 1-7) of the RNA construct of the invention (e.g. a saRNA replicon on the left, or a mRNA construct). The saRNA replicon (1-4) is based on an alpha virus backbone. This so-called ‘Stealthicon’ vector includes a 5′ UTR followed by nucleic acid encoding Non-structural Proteins (NSP1-4) from an alphavirus, such as VEEV, a sub-genomic promoter (SGP), a GOI (Gene of Interest), such as a viral, bacterial, fungal or mammalian protein or antigen, a non-viral innate modulatory protein (IMP), a 3′ UTR and a 3′ poly A tail. The mRNA construct (5-7) includes a 5′ UTR, a GOI (Gene of Interest), such as a viral, bacterial, fungal or mammalian protein or antigen, a non-viral innate modulatory protein (IMP), a 3′ UTR and a 3′ poly A tail. The order of the IMP and GOI can be varied for both saRNA and mRNA as shown in the different illustrated embodiments;

FIG. 2 illustrates the immune response in a subject vaccinated (an initial primer jab followed by a subsequent boost jab) with a messenger RNA (mRNA) vaccine;

FIG. 3 illustrates the immune response in a subject vaccinated (an initial primer jab followed by a boost jab) with a standard self-amplifying (saRNA) vaccine;

FIG. 4 illustrates the immune response in a subject vaccinated (an initial primer jab followed by a boost jab) with one embodiment of the RNA construct of the invention, for example the Stealthicon vector shown in FIG. 1;

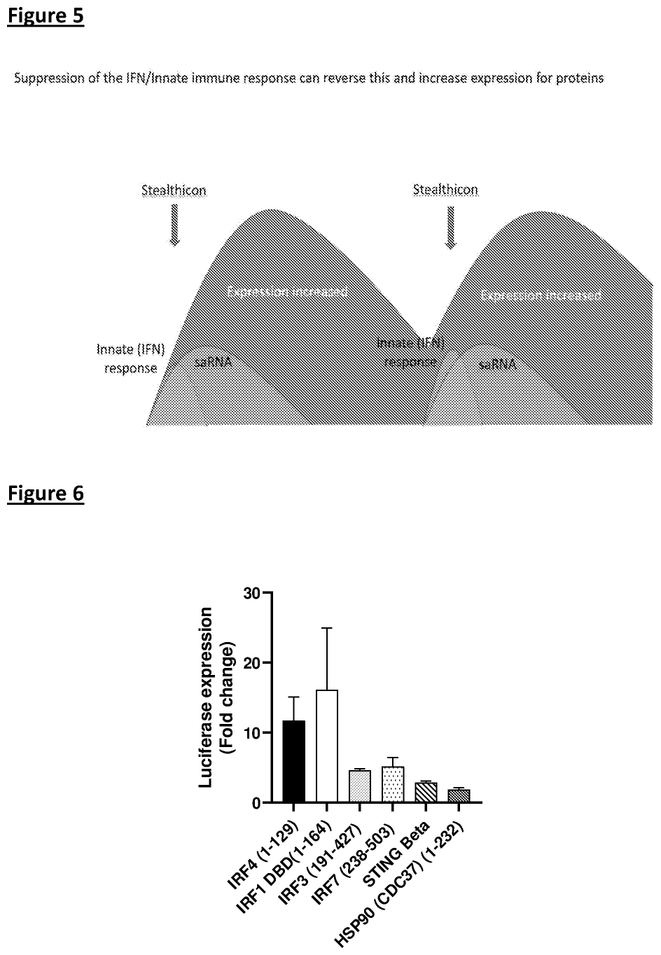

FIG. 5 illustrates the antigen expression level in a subject vaccinated (an initial primer jab followed by a boost jab) with one embodiment of the RNA construct of the invention, i.e. the Stealthicon vector shown in FIG. 1;

FIG. 6 shows f-Luc expression in HeLa cells following transfection with VEEV replicons containing selected IMP in F-T2A configuration relative to expression in HEK293T/17 cells. HEK293T/17 and HeLa cells were transfected with saRNA (100 ng) containing luciferase as a reporter protein and assessed for protein expression after 24 hr;

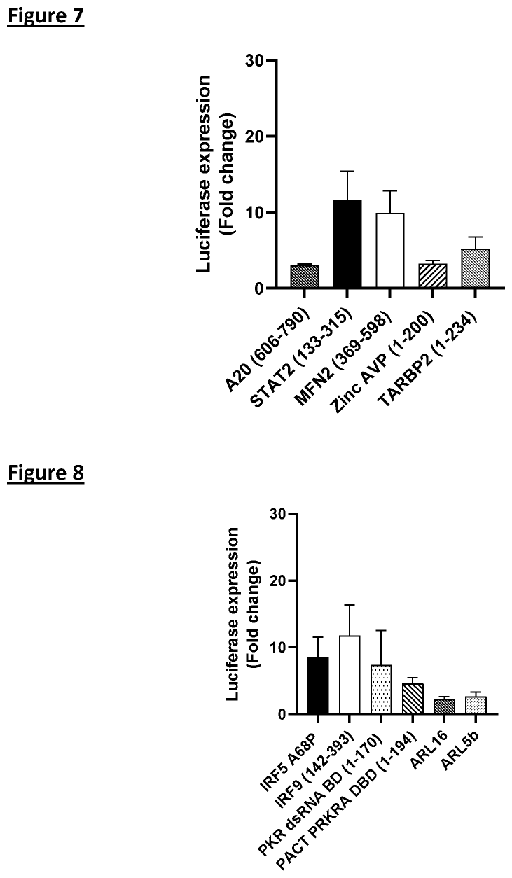

FIG. 7 shows f-Luc expression in HeLa cells following transfection with VEEV replicons containing selected IMP in F-T2A configuration relative to expression in HEK293T/17 cells. HEK293T/17 and HeLa cells were transfected with saRNA (100 ng) containing luciferase as a reporter protein and assessed for protein expression after 24 hr;

FIG. 8 shows f-Luc expression in HeLa cells following transfection with VEEV replicons containing selected IMP in F-T2A configuration relative to expression in HEK293T/17 cells. HEK293T/17 and HeLa cells were transfected with saRNA (100 ng) containing luciferase as a reporter protein and assessed for protein expression after 24 hr;

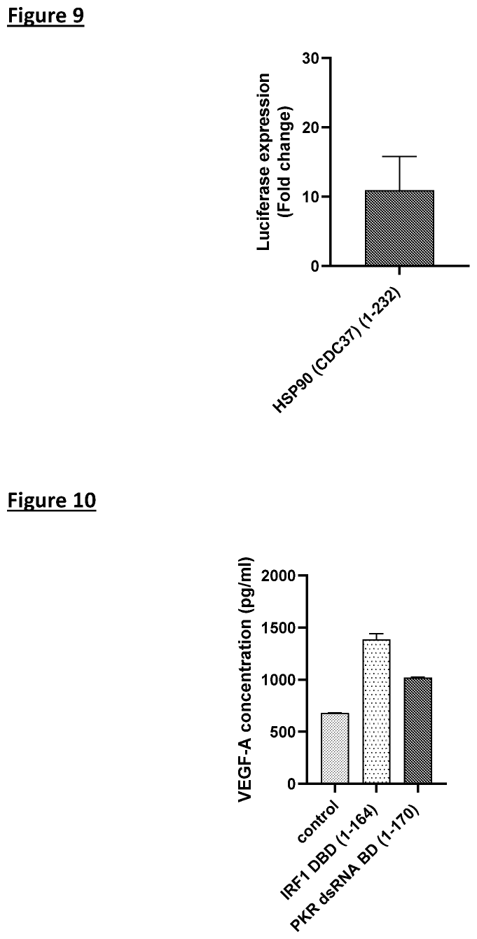

FIG. 9 shows f-Luc expression in HeLa cells following transfection with a VEEV replicon containing the IMP, HSP 90 CDC37 in a double sub-genomic promoter (DSGP) configuration relative to expression in HEK293T/17 cells. HEK293T/17 and HeLa cells were transfected with saRNA (100 ng) containing luciferase as a reporter protein and assessed for protein expression after 24 hr;

FIG. 10 shows the increase in VEGF-A expression produced in HeLa cells following transfection with saRNA containing the IMP in a F-T2A configuration compared to saRNA without IMP and relative to expression in HEK293T/17 cells. HEK293T/17 and HeLa cells were transfected with RNA (100 ng) containing VEGF-A as a secreted reporter protein and assessed for protein expression in the culture media after 48 hr by ELISA; and

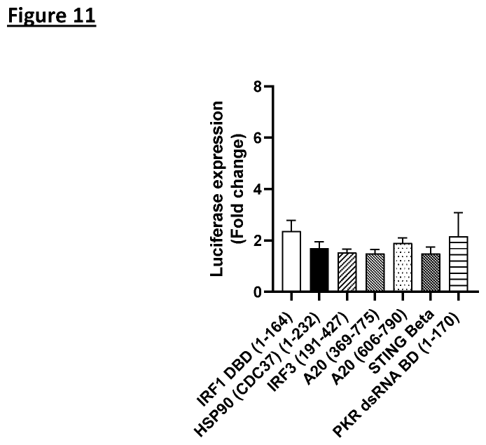

FIG. 11 shows n-Luc expression in HeLa cells following transfection with RNA containing IMP in an F-T2A configuration relative to expression in HEK293T/17 cells. HEK293T/17 and HeLa cells were transfected with RNA (100 ng) containing luciferase as a reporter protein and assessed for protein expression after 24 hr.

### EXAMPLES

The inventors hypothesized that cis encoding proteins from non-viral sources, such as humans and other mammals, that are known to inhibit the innate recognition of saRNA or mRNA, would dampen the innate sensing in the host cell, and enhance both the protein expression and immunogenicity of RNA vaccines. Thus, the inventors designed and tested a range of RNA constructs (saRNA and mRNA) containing innate modulatory proteins (IMPs) and a gene of interest (GOI), and then characterized whether these constructs enhance both intracellular and secreted protein expression (encoded by the gene of interest).

Materials and Methods

Cloning of saRNA Replicon Plasmids Containing IMPs

SaRNA encoding firefly luciferase (fLuc) and replicase derived from the Venezuelan equine encephalitis virus (VEEV) were cloned into a plasmid vector, as previously described (1). Replicon plasmids containing reporter gene followed by IMP (firefly luciferase f-Luc; Uniprot: Q27758) were generated with Furin-T2A or double sub-genomic promoters. Double sub-genomic (DSG) constructs are designed to initiate transcription of separate RNA molecules encoding the fLuc and IMP and were produced by cloning into a base double sub-genomic vector using Gibson assembly and a nucleotide base overlap. Briefly, plasmid DNA was restriction digested for 2 h at 37° C. and used in a NEB Builder HiFi DNA assembly reaction with gene fragment strings synthesised by GeneArt (Regensburg, Germany) or Integrated DNA Technologies (IDT) (Iowa, USA) according to manufacturer's protocol (New England BioLabs, UK). Furin-T2A (F-T2A) constructs designed to generate a single RNA transcript from the VEEV primary sub-genomic promoter with no stop codon for fLuc translation were produced by cloning IMP with F-T2A sequence into restriction enzyme sites of the corresponding DSG plasmid vector. After incubation at 50° C. for 30 min, 2 uL of the NEB Builder HiFi assembly reaction was used to transform NEB 10-alpha bacteria and the transformants plated onto LB agar plates and incubated overnight. Colonies were selected, expanded overnight and recombinant plasmid purified using Qiagen plasmid miniprep kits (Qiagen, UK). Purified clonal plasmids were analysed using a diagnostic restriction enzyme digest and those which exhibited the correct digestion pattern were fully sequenced to confirm nucleotide identity (Eurofins, Germany).

The incorporated interferon inhibiting proteins (IMP) can be found with the following database identifiers/accession numbers:

IRF1 DBD (1-164)—NCBI Reference Sequence: NM_002198.3, UniProtKB—P10914 (IRF1_HUMAN); IRF3 (191-427)—NCBI Reference Sequence: NM_001571.6, UniProtKB—Q14653 (IRF3_HUMAN); IRF7 (238-503)—NCBI Reference Sequence: NM_001572.5, UniProtKB—Q92985 (IRF7_HUMAN); IRF9 (142-393), IRF4 (1-129)—NCBI Reference Sequence: NM_002460.4, UniProtKB—Q15306 (IRF4_HUMAN); IRF5 A68P—NCBI Reference Sequence: NM_032643.5, UniProtKB—Q13568 (IRF5_HUMAN); STAT2 (133-315)—NCBI Reference Sequence: NM_005419.4, UniProtKB—P52630 (STAT2_HUMAN); HSP90 (CDC37) (1-232)—NCBI Reference Sequence: NM_007065.4, UniProtKB—Q16543 (CDC37_HUMAN); STING-Beta−GenBank: MF360993.1, UniProtKB—A0A3G1PSE3 (A0A3G1PSE3_HUMAN); A20 or TNFAIP3 (369-775), A20 or TNFAIP3 (606-790) NCBI Reference Sequence: NM_006290.4, UniProtKB—P21580 (TNAP3_HUMAN); MFN2 (369-598)—NCBI Reference Sequence: NM_001127660.2, UniProtKB—O95140 (MFN2_HUMAN); TARBP2 (1-234)—NCBI Reference Sequence: NM_134323.2, UniProtKB—Q15633 (TRBP2_HUMAN); Zinc finger AVP (1-200)—NCBI Reference Sequence: NM_020119.4, UniProtKB—Q7Z2W4 (ZCCHV_HUMAN); PKR dsRNA BD (1-170)—NCBI Reference Sequence: NM_002759.4, UniProtKB—P19525 (E2AK2_HUMAN); PACT PRKRA DBD (1-194)—NCBI Reference Sequence: NM_003690.5, UniProtKB—075569 (PRKRA_HUMAN); ARL5B—NCBI Reference Sequence: NM_178815.5, UniProtKB—Q96KC2 (ARL5B_HUMAN); ARL16—NCBI Reference Sequence: NM_001040025.3, UniProtKB—Q0P5N6 (ARL16_HUMAN), TRIM35—NCBI Reference Sequence NM_171982.4, UniProtKB—Q9UPQ4 (TRI35_HUMAN).
(1). A. K. Blakney, P. F. McKay, R. J. Shattock, Structural Components for Amplification of Positive and Negative Strand VEEV Splitzicons. Frontiers in Molecular Biosciences 5, 71 (2018).

Cloning of Plasmids Containing IMPs for RNA Transcription

IMP were inserted into a base plasmid using restriction digestion followed by Gibson assembly with a nucleotide base overlap region and included a F-T2A sequence to allow for a single transcript expression of the n-Luc followed by an IMP. The base plasmid consisted of an mRNA encoding a luminous shrimp nanoluciferase (n-Luc) expression cassette with a T7 promoter, an alpha-globin 5′ UTR and a beta-globin 3′ UTR. Briefly, the n-Luc plasmid construct was linearized with restriction enzymes for 2 h at 37° C. and then used in a NEB Builder HiFi DNA assembly reaction essentially as described in the NEB Builder HiFi assembly protocol (New England BioLabs, UK). After incubation at 50° C. for 30 min, 2 uL of the assembly reaction was used to transform NEB 10-alpha bacteria as per protocol and the transformants plated onto LB agar plates and incubated overnight for colony growth. Colonies were selected and expanded overnight, the recombinant plasmid purified from the bacteria using Qiagen plasmid miniprep kit (Qiagen, UK) and purified clonal plasmids were analysed initially using a diagnostic restriction enzyme digest and those which exhibited the correct digestion pattern were fully sequenced to confirm nucleotide identity (Eurofins, Germany).

In Vitro Transcription of saRNA

Plasmid DNA (pDNA) was transformed into Escherichia coli (E. coli) (New England BioLabs, UK) and cultured in 100 mL of Luria Broth (LB) with 100 μg/mL of carbenicillin (Sigma Aldrich, UK). pDNA was isolated using a Plasmid Plus MaxiPrep kit (QIAGEN, UK) and the final concentration measured on a NanoDrop One (ThermoFisher, UK). saRNA was transcribed from the pDNA template using CleanCap Reagent AG (Tebu-bio, France) to produce an RNA transcript with a naturally occurring Cap 1 structure. Briefly, the pDNA template was linearized for 3 h at 37° C., then 1 μg of the linearized pDNA template used in the standard CleanCap Transcription protocol (Tebu-bio, France) according to the manufacturer's protocol. Transcripts were purified by LiCl precipitation at −20° C. for at least 30 min, centrifuged at 20,000 g for 20 min at 4° C. to pellet the RNA, rinsed once with 70% EtOH, centrifuged again at 20,000 g for 5 min at 4° C. and resuspended in UltraPure H₂O (Ambion, UK) and stored at −80° C. until further use.

In Vitro Transcription of RNA

pDNA was transformed into E. coli (New England BioLabs, UK), cultured in 100 mL of Luria Broth (LB) with 100 μg/mL of carbenicillin (Sigma Aldrich, UK). Plasmid was purified using a Plasmid Plus MaxiPrep kit (QIAGEN, UK) and the concentration and purity measured on a NanoDrop One (ThermoFisher, UK). RNA was transcribed from the plasmid DNA template using the MEGAscript™ T7 Transcription protocol (ThermoFisher, UK) followed by a ScriptCap™ m7G Capping System post translation (Cambio, UK). Briefly, pDNA was linearized for 3 h at 37° C., and 1 μg of the linearized pDNA template used in the standard reaction protocol. After the MEGAscript™ T7 Transcription the transcripts were purified by LiCl precipitation at −20° C. for at least 30 min, then centrifuged at 20,000 g for 20 min at 4° C. to pellet the RNA, rinsed once with 70% EtOH, centrifuged again at 20,000 g for 5 min at 4° C. and resuspended in UltraPure H₂O (Ambion, UK). The transcripts were then post-transcriptionally capped using the ScriptCap™ m7G Capping System standard protocol and finally LiCi precipitated as described above. Purified and Cap 1 capped RNA was then resuspended in UltraPure H₂O (Ambion, UK) and stored at −80° C. until further use.

Measurement of IMP Activity

In order to establish the ability of saRNA containing viral IMP to increase saRNA f-luc expression relative to saRNA without IMP; the ability of mRNA containing IMP to increase mRNA n-luc expression relative to mRNA without IMP and the ability of mRNA containing IMP to increase f-luc expression from saRNA without IMP, constructs were tested in interferon competent HeLa cells and expression compared to that obtained in HEK293T/17 cells which do not have a functional antiviral signalling pathway. Both cell lines were cultured in high glucose Dulbecco's Modified Eagle's Medium (cDMEM) (Sigma-Aldrich, Merck, UK) containing 10% (v/v) fetal bovine serum (FBS), 5 mg/mL L-glutamine (Gibco, ThermoFisher, UK) and 5 mg/mL penicillin/streptomycin (Sigma-Aldrich, Merck, UK).

Assessment of IMP on saRNA Firefly Luciferase (f-Luc) Expression

HEK293T/17 cells were plated at a density of 25000 cells per well and HeLa cells at a density of 10000 cells per well into flat clear bottom 96-well plates (Corning Costar) and incubated for 24 hr. 10 uL of OptiMEM (ThermoFisher, UK) containing 0.15 μL lipofectamine MessengerMAX (ThermoFisher, UK) and 100 ng of saRNA IMP constructs or saRNA control (no IMP) was added to triplicate wells and after a further 24 hr, plates were centrifuged at 630 g for 5 min at room temperature, 50 μL of medium removed from each well and 50 μL of ONE-Glo™ Ex Reagent D-luciferin reagent (Promega, UK) added and mixed by pipetting. The total volume from each well was then transferred to a flat bottom opaque white 96-well plate (Corning Costar) and fluorescence measured on a FLUOstar OMEGA plate reader within 10 min (BMG LABTECH, UK). Background fluorescence from control wells containing no saRNA was subtracted from the signal for each well containing saRNA. Then the signal obtained for saRNA containing IMP in HeLa cells was expressed as a fold change from signal obtained with control saRNA and to that obtained in HEK293T/17 cells.

Assessment of IMP on RNA Nano-Luciferase (n-Luc) Expression

HEK293T/17 cells were plated at a density of 25000 cells per well and HeLa cells at a density of 10000 cells per well into flat clear bottom 96-well plates (Corning Costar) and incubated for 24 hr. 10 uL of OptiMEM (ThermoFisher, UK) containing 0.15 μL lipofectamine and 100 ng of saRNA IMP constructs or saRNA control (no IMP) was added to triplicate wells and after a further 24 hr, plates were centrifuged at 630 g for 5 min at room temperature, 50 μL of medium removed from each well and 50 μL of NanoDLR™ Stop & Glo® Reagent (Promega, UK) added and mixed by pipetting. The total volume from each well was then transferred to a flat bottom opaque white 96-well plate (Corning Costar) and fluorescence measured on a FLUOstar® OMEGA plate reader within 10 min (BMG LABTECH, UK). Background fluorescence from control wells containing no RNA was subtracted from the signal for each well containing RNA. Then the signal obtained for RNA containing IMP in HeLa cells was expressed as a fold change from signal obtained with control RNA and to that obtained in HEK293T/17 cells.

Assessment of IMP on saRNA VEGF-A Expression

HEK or Hela cells were transfected with 100 ng saRNA containing the VEGF-A gene using the same methods as described for testing of constructs expressing f-Luc. After 48 hr the VEGF-A in the cell culture media was measured using a human VEGF-A ELISA kit (Invitrogen, UK). Briefly, assay plate wells were washed twice with 400 uL wash buffer before addition of test samples or VEGF-A standard (15.6 μg/ml to 1000 pg/ml). Plates were then incubated at room temperature for 2 hr in a microplate shaker (300 rpm; Jencons Scientific Ltd, UK) before washing six times with 400 uL wash buffer 100 uL of Biotin-conjugate detection antibody (1:100 dilution) was added to each well and plates incubated in a microplate shaker (1 hr RT, 300 rpm). After six washes with 400 uL of wash buffer, the streptavidin-HRP (1:100 dilution) second layer conjugate (100 uL) was added and after a further 1 hr incubation and six further washes, 100 uL of TMB subtrate was added to each well. After incubation int the dark for 30 min at RT in the dark, 100 uL of the Stop solution was added and the absorbance of each well read at 450 nm in a VersaMax microplate spectrophotometer (Molecular Devices, UK). VEGF-A levels in the samples were determined by interpolation to the standard curve.

### Example 1—Structural Design of Innate Modulatory Protein (IMP) Constructs

Human innate modulatory proteins (IMPs) can be incorporated into an RNA construct of the invention, which can be a self-amplifying RNA (saRNA) or a messenger RNA (mRNA)system, in order to reduce or ablate the innate recognition and response that may modify or reduce protein expression and translation, i.e. the protein encoded by a Gene of Interest (GOI), which can be any therapeutic biomolecule.

Various embodiments of design configurations for the RNA construct of the invention are shown in FIG. 1. SaRNA expression constructs are based on an alphavirus backbone where the non-structural proteins are maintained, but the gene of interest (GOI) is inserted downstream of a subgenomic promotor (SGP) replacing the structural genes of the virus (see Embodiment “1” in FIG. 1). The GOI can be any protein at all, and may include viral, bacterial, fungal or mammalian protein, i.e. a biotherapeutic protein. However, the inventors envisage that the RNA construct of the invention will demonstrate significant utility in the vaccine space, and so the GOI would encode a vaccine antigen, such as a viral, bacterial or fungal protein, such as a coat protein.

saRNA Constructs (Left Hand of FIG. 1)

Any IMP can be encoded within the saRNA using the following design approaches:
Embodiment “2a” in FIG. 1 shows a saRNA construct encoding a fusion protein including a peptide cleavage motif (e.g. furin-T2a), such that the protein encoded by the GOI (e.g. the antigen of interest) and the IMP are cleaved into separate proteins on translation in the host cell;
In Embodiment “2b” in FIG. 1, the order of the GOI and IMP have been reversed, such that the IMP is 5′ of the GOI, again with a peptide cleavage motif between the IMP and the GOI so that two separate proteins are produced in the host cell following translation of the saRNA construct;
In Embodiment “3a”, the IMP has been inserted downstream of the GOI stop codon. The subgenomic promoter drives translation of the GOI, and expression/translation of the IMP is driven by the inclusion of an internal ribosomal entry site (IRES);
In Embodiment “3b”, the order of the GOI and IMP has been reversed such that translation of the IMP is promoted by the subgenomic promotor and of the GOI by the IRES;
In Embodiment “4a”, the IMP has been inserted downstream of the GOI stop codon. Translation of the GOI is promoted by the first subgenomic promoter and translation of the IMP is driven by the inclusion of a second subgenomic promotor;
In Embodiment “4b”, the position of the IMP and GOI have been swapped around, i.e. with the IMP placed before the GOI.

mRNA Constructs (Right Hand of FIG. 1)

Referring to FIG. 1, any IMP can also be encoded within mRNA (see Embodiment “5”) using the following design approaches:
In embodiment “6a”, the mRNA construct encodes a fusion protein including a peptide cleavage motif (e.g. F-T2a) such that the GOI and IMP are cleaved into separate proteins on translation;
In Embodiment “6b”, the order of the GOI and IMP have been reversed such that the IMP is 5′ of the GOI;
In Embodiment “7a”, the IMP has been inserted downstream of the GOI stop codon where translation is driven by the inclusion of an internal ribosomal entry site (IRES);
In Embodiment “7b”, the order of the GOI and IMP has been reversed such that translation is promoted by the subgenomic promotor and the GOI by the IRES.

The inventors have tested a large number of human IMPs in the various embodiments of RNA constructs illustrated in FIG. 1, and believe that they each have potential to modify expression and response to saRNA and/or mRNA.

### Example 2—Construction and Testing saRNA Constructs Comprising Non-Viral Innate Modulatory Protein (IMP)

The inventors designed, constructed and then tested a series of diverse non-viral IMPs, and the results of the expression studies are shown in FIGS. 6-10.

Referring to FIG. 6, there is shown the fold increase in f-Luc expression in HeLa cells following transfection with VEEV replicons containing the IMP; IRF4 (1-129), IRF1 DBD (1-164), IRF3 (191-427), IRF7 (238-503), STING beta and HSP90 (CDC37) (1-232) in an F-T2A configuration. HEK293T/17 and HeLa cells were transfected with saRNA (100 ng) containing luciferase as a reporter protein and assessed for protein expression after 24 hr. HeLa cells are known to have more intact IFN expression pathways compared to HEK and therefore increased expression (fold increase) relative to a control (saRNA containing luciferase as reporter protein and no IIP) indicates that the IIP is increasing saRNA expression. Of these IMP; IRF1 DBD (1-164) and IRF4(1-129) produced the greatest increase in f-Luc expression. Data shown are constructs providing a greater than ˜2-fold increase in luciferase expression in HeLa cells relative to expression in HEK293T/17 cells and are mean f SEM of data obtained in 3 independent experiments using 3 separate batches of saRNA.

Referring to FIG. 7, there is shown f-Luc expression in HeLa cells following transfection with VEEV replicons containing A20(606-790), STAT2(133-315), MFN2 (369-598), Zinc finger AVP (1-200) and TARBP2(1-234) in an F-T2A configuration relative to expression in HEK293T/17 cells. Details of experimental methods are provided in FIG. 6. Of these STAT2(133-315), MFN2 (369-598) produced the greatest increase in f-Luc expression. Data shown are constructs providing a greater than 2-fold increase in luciferase expression in HeLa cells relative to expression in HEK293T/17 cells and are mean f SEM of data obtained in 3 independent experiments using 3 separate batches of saRNA.

Referring to FIG. 8, there is shown f-Luc expression in HeLa cells following transfection with VEEV replicons containing IRF5 A68P, IRF9 (142-393), PKR dsRNA BD (1-170) and PACT PRKRA DBD (1-194), ARL5B and ARL16 in an F-T2A configuration relative to expression in HEK293T/17 cells. Details of experimental methods are provided in FIG. 6. Of these IRF9 (142-393) produced the greatest increase in f-Luc expression. Data shown are constructs providing a greater than 2-fold increase in luciferase expression in HeLa cells relative to expression in HEK293T/17 cells and are mean f SEM of data obtained in 3 independent experiments using 3 separate batches of saRNA.

Referring to FIG. 9, there is shown f-Luc expression in HeLa cells following transfection with VEEV replicons the IMP, HSP 90 CDC37 in a double sub-genomic promoter (DSGP) configuration relative to expression in HEK293T/17 cells. Details of experimental methods are provided in FIG. 6. Data shown is luciferase expression in HeLa cells relative to expression in HEK293T/17 cells and are mean f SEM of data obtained in 3 independent experiments using 3 separate batches of saRNA.

FIG. 10 shows the increase in VEGF-A expression produced in HeLa cells following transfection with saRNA containing IRF1 DBD (1-164) or PKR dsRNA BD (1-170) in a F-T2A configuration compared to saRNA without IMP and relative to expression in HEK293T/17 cells. HEK293T/17 and HeLa cells were transfected with RNA (100 ng) containing VEGF-A as a secreted reporter protein and assessed for protein expression in the culture media after 48 hr by ELISA. HeLa cells are known to have more intact IFN expression pathways compared to HEK and therefore increased expression relative to a control (RNA containing VEGF-A as GOI and no IIP) indicates that the IIP is increasing RNA expression. Data are from one experiment and represent the mean f SEM of three replicate measurements.

### Example 3—Construction and Testing RNA Constructs Comprising Non-Viral Innate Modulatory Protein (IMP)

The inventors designed, constructed and then tested a series of diverse non-viral IMPs, and the results of the expression studies are shown in FIG. 11.

Referring to FIG. 11, there is shown n-Luc expression in HeLa cells following transfection with RNA containing the IMPs: IRF1 DBD (1-164), HSP90 (CDC37) (1-232), IRF3(191-427), A20(369-775), A20(606-790), STING Beta and PKR dsRNA BD (1-170) in an F-T2A configuration relative to expression in HEK293T/17 cells. Details of experimental methods are provided in FIG. 6. Data shown are constructs providing a greater than ˜2-fold increase in luciferase expression and are mean f SEM of data obtained in 3 independent experiments using 3 separate batches of RNA.

### CONCLUSIONS

The inventors believe that the constructs described herein display many advantages over those described in the prior art, including:
i) insertion of nucleotide sequences encoding any of the innate modulatory proteins directly into the RNA construct, such as mRNA or saRNA, enabling dual protein expression of the IMP protein and the biotherapeutic molecule encoded by the gene of interest;
ii) as opposed to delivering two different and separate strands of RNA, one encoding the gene of interest (GOI), i.e. the therapeutic biomolecule, and one encoding the IMP, a single strand is delivered;
iii) the IMP inhibits innate sensing of RNA, thus enabling higher protein expression;
iv) when the RNA construct is a saRNA, the IMP expression itself is self-amplified by virtue of being co-expressed on the sub-genome strand with the GOI; and/or
v) an increase in both the magnitude and duration of protein expression compared to conventional VEEV RNA replicon constructs.

### Numbered Paragraphs

The following paragraphs form part of the description and not the claims.

1. An RNA construct encoding: (i) at least one therapeutic biomolecule; and (ii) at least one non-viral innate modulatory protein (IMP).

2. The RNA construct according to paragraph 1, wherein the construct comprises mRNA, saRNA or a trans-replicon system, and preferably saRNA.

3. The RNA construct according to either paragraph 1 or paragraph 2, wherein the saRNA construct comprises or is derived from a positive stranded RNA virus selected from the group of genus consisting of: alphavirus; picornavirus; flavivirus; rubivirus; pestivirus; hepacivirus; calicivirus and coronavirus, preferably an alphavirus, optionally VEEV.

4. The RNA construct according to any preceding paragraph, wherein the IMP is a mammalian IMP, preferably a human IMP.

5. The RNA construct according to any preceding paragraph, wherein the innate modulatory protein encoded by the RNA construct comprises a mutated or non-functional interferon regulatory factor (IRF), or a dominant negative form thereof.

6. The RNA construct according to paragraph 5, wherein the mutated or non-functional interferon regulatory factor, or dominant negative form thereof, is any one of IRF1, IRF2, IRF3, IRF4, IRF5, IRF6, IRF7, IRF8, or IRF9, or an orthologue thereof.

7. The RNA construct according to either paragraph 5 or 6, wherein the innate modulatory protein encoded by the RNA construct comprises an interferon regulatory factor (IRF), which has had its DNA binding domain (DBD) and/or Nuclear Location Signal (NLS) rendered non-functional or deleted, so that it becomes a dominant negative form in the cytoplasm.

8. The RNA construct according to any preceding paragraph, wherein the mutated or non-functional interferon regulatory factor, or dominant negative form thereof, may comprise or consist of the DNA binding domain (DBD) and/or the Nuclear Location Signal (NLS) of an interferon regulatory factor (IRF).

9. The RNA construct according to any preceding paragraph, wherein the at least one IMP is a dominant negative form of IRF and is selected from a group consisting of: IRF1 dominant negative; IRF3 dominant negative; IRF7 dominant negative; and IRF9 dominant negative.

10. The RNA construct according to any preceding paragraph, wherein the at least one IMP is the DBD of an IRF selected from a group consisting of: IRF1; IRF4; IRF5; IRF8; and IRF9, or an orthologue thereof.

11. The RNA construct according to any one of paragraphs 1 to 4, wherein the innate modulatory protein encoded by the RNA construct comprises a mutated or non-functional inhibitor of an innate signalling pathway, or a dominant negative form thereof.

12. The RNA construct according to any one of paragraphs 1 to 4, wherein the innate modulatory protein encoded by the RNA construct comprises a mutated or non-functional inhibitor of RNA recognition, or a dominant negative form thereof.

13. The RNA construct according to any one of paragraphs 1 to 4, wherein the at least one IMP may be selected from: RIG-1, FAF1, SOCS1, SOCS3, USP18, USP21 and USP27, or an orthologue thereof.

14. The RNA construct according to any one of paragraphs 1 to 4, wherein the at least one IMP may be selected from: CYLD, LGP2, RIG splice variant, DDX-56, ARL16 and ARL5B, or an orthologue thereof.

15. The RNA construct according to any preceding paragraph, wherein the therapeutic biomolecule comprises a therapeutic protein, preferably the protein or peptide is an antigen, and more preferably a viral antigen.

16. A nucleic acid sequence encoding the RNA construct according to any preceding paragraph.

17. An expression cassette comprising a nucleic acid sequence according to paragraph 16.

18. A recombinant vector comprising the expression cassette according to paragraph 17.

19. A pharmaceutical composition comprising the RNA construct according to any one of paragraphs 1 to 15, the nucleic acid sequence according to paragraph 16, the expression cassette according to paragraph 17 or the vector according to paragraph 18, and a pharmaceutically acceptable vehicle.

20. A method of preparing the RNA construct according to any one of paragraphs 1 to 15, the method comprising:
a) i) introducing, into a host cell, the vector according to paragraph 18; and
ii) culturing the host cell under conditions to result in the production of the RNA construct according to any one of paragraphs 1 to 15; or
b) transcribing the RNA construct from the vector according to paragraph 18.

21. The RNA construct according to any one of paragraphs 1 to 15, the nucleic acid sequence according to paragraph 16, the expression cassette according to paragraph 17 or the vector according to paragraph 18 or the pharmaceutical composition according to paragraph 19, for use as a medicament or in therapy.

22. The RNA construct according to any one of paragraphs 1 to 15, the nucleic acid sequence according to paragraph 16, the expression cassette according to paragraph 17 or the vector according to paragraph 18 or the pharmaceutical composition according to paragraph 19, for use in the prevention, amelioration or treatment of a protozoan, fungal, bacterial or viral infection.

23. The RNA construct according to any one of paragraphs 1 to 15, the nucleic acid sequence according to paragraph 16, the expression cassette according to paragraph 17 or the vector according to paragraph 18 or the pharmaceutical composition according to paragraph 19, for use in the prevention, amelioration or treatment of cancer.

24. A vaccine comprising the RNA construct according to any one of paragraphs 1 to 15, the nucleic acid sequence according to paragraph 16, the expression cassette according to paragraph 17 or the vector according to paragraph 18 or the pharmaceutical composition according to paragraph 19.

25. The RNA construct according to any one of paragraphs 1 to 15, the nucleic acid sequence according to paragraph 16, the expression cassette according to paragraph 17 or the vector according to paragraph 18 or the pharmaceutical composition according to paragraph 19, for use in stimulating an immune response in a subject.

1. An RNA construct encoding: (i) at least one therapeutic biomolecule; and (ii) at least one non-viral innate modulatory protein (IMP).

2. The RNA construct according to claim 1, wherein the construct comprises mRNA.

3. The RNA construct according to claim 1, wherein the construct comprises saRNA.

4. The RNA construct according to claim 1, wherein the saRNA construct comprises or is derived from a positive stranded RNA virus selected from the group of genus consisting of: alphavirus; picornavirus; flavivirus; rubivirus; pestivirus; hepacivirus; calicivirus and coronavirus, preferably an alphavirus, optionally VEEV.

5. The RNA construct according to claim 1, wherein the IMP is a mammalian IMP, preferably a human IMP.

6. The RNA construct according to claim 1, wherein the IMP is configured to inhibit interferon regulatory factor activity.

7. The RNA construct according to claim 6, wherein the IMP is selected from: IRF1 DBD (1-164), IRF9 (142-393), IRF4 (1-129), IRF5 A68P, IRF3 (191-427), IRF7 (238-503), IRF2 (1-113), IRF9 (1-120), IRF4(21-129), IRF9 (182-235), IRF9(200-308), IRF5(1-140), IRF6(1-115), IRF8(1-140), and/or IRF1 (141-325); preferably wherein the IMP is selected from: IRF1 DBD (1-164), IRF9 (142-393), IRF4 (1-129), IRF5 A68P, IRF3 (191-427) and/or IRF7 (238-503).

8. The RNA construct according to claim 1, wherein the IMP is configured to inhibit a pathway leading to interferon production and resulting in stimulation of interferon-stimulated genes.

9. The RNA construct according to claim 8, wherein the IMP is selected from: HSP90 (CDC37) (1-232), STING Beta, MFN2 (369-598), A20(606-790), A20(369-775), ARL5B, ARL16, FAF1, MFN2 (1-757), USP21, USP27, CYLD, LGP2, DDX-56, MAVS (ΔCARD domain), TRIM35, MFN2(400-480), and/or MFN2 (369-490); preferably wherein the IMP is selected from: HSP90 (CDC37) (1-232), STING Beta, MFN2 (369-598), A20(606-790), A20(369-775), and/or ARL5B, ARL16.

10. The RNA construct according to claim 1, wherein the IMP is configured to inhibit interferon signalling.

11. The RNA construct according to claim 10, wherein the IMP is selected from: STAT2 (133-315), IRF9 (142-393), STAT1 DN, STAT2 (1-851-F175DY701F), USP18, SOCS1, and/or SOCS3; preferably wherein the IMP is selected from: STAT2 (133-315), and/or IRF9 (142-393).

12. The RNA construct according to claim 1, wherein the IMP is configured to inhibit RNA recognition systems.

13. The RNA construct according to claim 12, wherein the IMP is selected from: Zinc AVP (1-200), TARBP2 (1-234), PKR dsRNA BD (1-170), PACT PRKRA BD (1-194), OAS3 Domain 1, RNAse L dominant negative, and/or a RIG-1 dominant negative or splice variant; preferably wherein the IMP is selected from: Zinc AVP (1-200), TARBP2 (1-234), PKR dsRNA BD (1-170) and/or PACT PRKRA BD (1-194).

14. The RNA construct according to claim 1, wherein the therapeutic biomolecule comprises a therapeutic protein, preferably the protein or peptide is an antigen, and more preferably a viral antigen.

15. A nucleic acid sequence encoding the RNA construct according to claim 1.

16-24. (canceled)
